(19) United States
(12) Patent Application Publication
(10) Pub. No.: US 2021/0028373 A1
(43) Pub. Date: Jan. 28, 2021

(54) A PLURALITY OF HOST MATERIALS AND ORGANIC ELECTROLUMINESCENT DEVICE COMPRISING THE SAME

(71) Applicant: ROHM AND HAAS ELECTRONIC MATERIALS KOREA LTD., Chungcheongnam-do (KR)

(72) Inventors: Bitnari KIM, Gyeonggi-do (KR); Su-Hyun LEE, Gyeonggi-do (KR); So-Young JUNG, Gyeonggi-do (KR); Doo-Hyeon MOON, Gyeonggi-do (KR); Du-Yong PARK, Gyeonggi-do (KR); Sang-Hee CHO, Gyeonggi-do (KR); Hae-Yeon KIM, Gyeonggi-do (KR)

(21) Appl. No.: 16/924,332

(22) Filed: Jul. 9, 2020

(30) Foreign Application Priority Data
Jul. 18, 2019 (KR) 10-2019-0087143
May 13, 2020 (KR) 10-2020-0056910

(51) Int. Cl. H01L 51/00; C07D 487/04; C09K 11/06; C07D 209/94; C07D 487/16; C07D 409/04; C07D 405/10; C07D 409/10; C07D 251/24

(52) U.S. Cl. CPC H01L 51/0072; H01L 51/5012; C09K 11/06; H01L 51/0073; C07D 209/94; C07D 487/16; C07D 409/04; H01L 51/0067; H01L 51/0074; C07D 405/10; C07D 409/10; C07D 251/24; H01L 51/0054; H01L 51/0058; C07D 487/04

(57) ABSTRACT

The present disclosure relates to a plurality of host materials comprising a first host material comprising a compound represented by formula 1, and a second host material comprising a compound represented by formula 2, and an organic electroluminescent device comprising the same. By comprising a specific combination of compounds as a host material, it is possible to provide an organic electroluminescent device having an improved lifespan property, compared to conventional organic electroluminescent devices.

### TECHNICAL FIELD

The present disclosure relates to a plurality of host materials and an organic electroluminescent device comprising the same.

### BACKGROUND ART

A small molecular green organic electroluminescent device (OLED) was first developed by Tang, et al., of Eastman Kodak in 1987 by using TPD/ALq3 bi-layer consisting of a light-emitting layer and a charge transport layer. Thereafter, the development of OLEDs was rapidly effected and OLEDs have been commercialized. At present, OLEDs primarily use phosphorescent materials having excellent luminous efficiency in panel implementation.

However, in many applications such as TVs and lightings, OLED lifetime is insufficient and higher efficiency of OLEDs is still required. Typically, the higher the luminance of an OLED is, the shorter the lifetime of an OLED is. Therefore, an OLED having high luminous efficiency and/or long lifespan characteristics is required for long time use and high resolution of a display.

In order to enhance luminous efficiency, driving voltage and/or lifespan, various materials or concepts for an organic layer of an organic electroluminescent device have been proposed. However, they were not satisfactory in practical use.

Korean Patent Application Laying-Open No. 2019-0013353 discloses an organic optoelectronic device using a compound having a benzonaphto-based heteroaryl moiety with a compound having an indolocarbazole moiety as hosts of a light-emitting layer. However, an improvement of performances of the device is required.

### DISCLOSURE OF INVENTION

### Technical Problem

The objective of the present disclosure is to provide an organic electroluminescent device having an improved lifespan property by comprising a plurality of host materials including a specific combination of compounds.

### Solution to Problem

As a result of intense studies, the present inventors found that the above objective can be achieved by a plurality of host materials comprising a first host material comprising a compound represented by the following formula 1, and a second host material comprising a compound represented by the following formula 2:

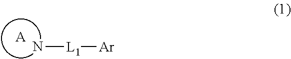

wherein

L₁ represents a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene; Ar represents hydrogen, deuterium, a halogen, a cyano, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, a substituted or unsubstituted (3- to 30-membered)heteroaryl, —NR₁R₂, or —SiR₃R₄R₅;

R₁ to R₅, each independently, represent a substituted or unsubstituted (C1-C30)alkyl, a substituted or unsubstituted (C6-C30)aryl, or a substituted or unsubstituted (3- to 30-membered)heteroaryl;

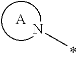

is represented by any one of the following formulas 1-1 to 1-7:

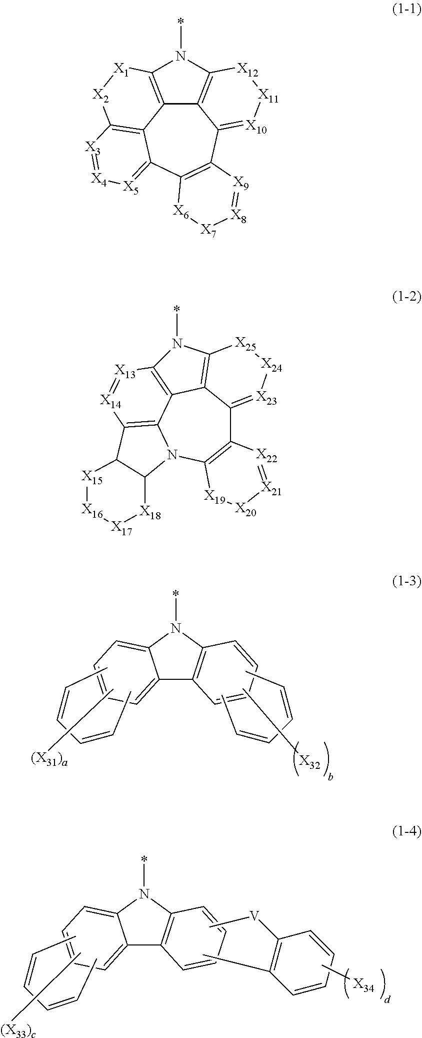

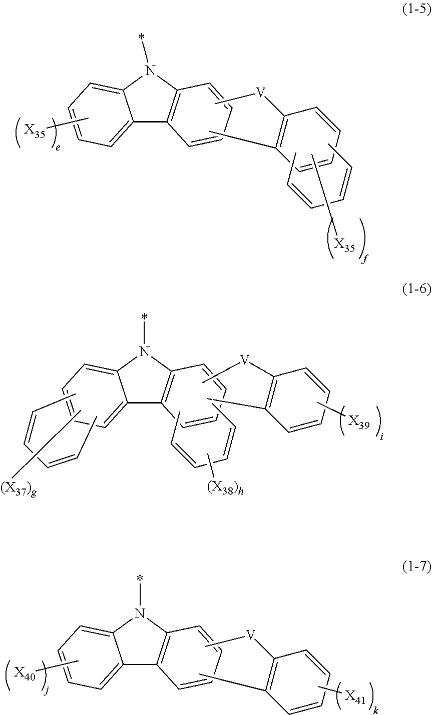

X₁ to X₂₅, each independently, represent N or CR₆;

R₆, each independently, represents hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or adjacent R₆'s may be linked to each other to form a ring(s), and if two or more R₆'s are present, each of R₆ may be the same or different;

V represents CX₂₆X₂₇, NX₂₈, O, or S;

X₂₆ to X₂₈ and X₃₁ to X₄₁, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or each of X₃₁ to X₄₁ may be linked to an adjacent substituent of X₃₁ to X₄₁ to form a ring(s);

a, b, c, f, and g, each independently, represent an integer of 1 to 6, and d, e, h, i, j, and k, each independently, represent an integer of 1 to 4, where if a to k are an integer equal to 2 or more, each of X₃₁ to each of X₄₁ may be the same or different; and

* represents a bonding site to L₁;

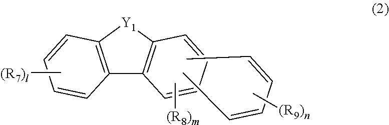

wherein

Y₁ represents O, S, CR₁₁R₁₂, or NR₁₃;

R₁₁ to R₁₃, each independently, represent hydrogen, deuterium, a halogen, a cyano, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, or a substituted or unsubstituted (3- to 30-membered)heteroaryl; or R₁₁ and R₁₂ may be linked to each other to form a ring;

R₇ to R₉, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted (C1-C30)alkyl(C6-C30)arylamino, or a substituted or unsubstituted mono- or di-(C6-C30)arylamino; or may be linked to an adjacent substituent to form a ring(s);

with the proviso that at least one of R₁₃, R₈, and R₉ represents -L₂-(Ar₁)ₚ;

L₂ represents a single bond, a substituted or unsubstituted (C6-C30)arylene, or a substituted or unsubstituted (3- to 30-membered)heteroarylene;

Ar₁, each independently, represents a substituted or unsubstituted (3- to 30-membered)heteroaryl containing at least one nitrogen; and

l and n, each independently, represent an integer of 1 to 4, and m and p, each independently, represent an integer of 1 or 2, where if l to n and p are an integer equal to 2 or more, each of R₇, each of R₈, each of R₉, and each of Ar₁ may be the same or different.

### Advantageous Effects of Invention

By comprising a plurality of host materials according to the present disclosure, an organic electroluminescent device having a property of improved lifespan compared to conventional organic electroluminescent devices can be provided, and it is possible to produce a display device or a lighting device using the same.

### MODE FOR THE INVENTION

Hereinafter, the present disclosure will be described in detail. However, the following description is intended to explain the invention and is not meant in any way to restrict the scope of the present disclosure.

The term “organic electroluminescent material” in the present disclosure means a material that may be used in an organic electroluminescent device, and may comprise at least one compound. The organic electroluminescent material may be comprised in any layer constituting an organic electroluminescent device as necessary. For example, the organic electroluminescent material may be a hole injection material, a hole transport material, a hole auxiliary material, a light-emitting auxiliary material, an electron blocking material, a light-emitting material (including a host material and a dopant material), an electron buffer material, a hole blocking material, an electron transport material, an electron injection material, etc.

The term “a plurality of organic electroluminescent materials” in the present disclosure means an organic electroluminescent material comprising a combination of at least two compounds, which may be comprised in any layer constituting an organic electroluminescent device. It may mean both a material before being comprised in an organic electroluminescent device (for example, before vapor deposition) and a material after being comprised in an organic electroluminescent device (for example, after vapor deposition). For example, a plurality of organic electroluminescent materials may be a combination of at least two compounds, which may be comprised in at least one layer of a hole injection layer, a hole transport layer, a hole auxiliary layer, a light-emitting auxiliary layer, an electron blocking layer, a light-emitting layer, an electron buffer layer, a hole blocking layer, an electron transport layer, and an electron injection layer. Such at least two compounds may be comprised in the same layer or different layers through methods used in the art, and for example may be mixture-evaporated or co-evaporated, or may be individually evaporated.

The term “a plurality of host materials” in the present disclosure means a host material comprising a combination of at least two compounds, which may be comprised in any light-emitting layer constituting an organic electroluminescent device. It may mean both a material before being comprised in an organic electroluminescent device (for example, before vapor deposition) and a material after being comprised in an organic electroluminescent device (for example, after vapor deposition). For example, a plurality of host materials of the present disclosure may be a combination of at least two host materials, and selectively may further comprise conventional materials comprised in an organic electroluminescent material. A plurality of host materials of the present disclosure may be comprised in any light-emitting layer constituting an organic electroluminescent device, and at least two compounds comprised in the plurality of host materials of the present disclosure may be comprised together in one light-emitting layer or may respectively be comprised in different light-emitting layers, through methods used in the art. For example, the at least two compounds may be mixture-evaporated or co-evaporated, or may be individually evaporated.

Herein, the term “(C1-C30)alkyl(ene)” is meant to be a linear or branched alkyl having 1 to 30 carbon atoms constituting the chain, in which the number of carbon atoms is preferably 1 to 20, and more preferably 1 to 10. The above alkyl may include methyl, ethyl, n-propyl, iso-propyl, n-butyl, iso-butyl, tert-butyl, etc. The term “(C2-C30)alkenyl” is meant to be a linear or branched alkenyl having 2 to 30 carbon atoms constituting the chain, in which the number of carbon atoms is preferably 2 to 20, and more preferably 2 to 10. The above alkenyl may include vinyl, 1-propenyl, 2-propenyl, 1-butenyl, 2-butenyl, 3-butenyl, 2-methylbut-2-enyl, etc. The term “(C2-C30)alkynyl” is meant to be a linear or branched alkynyl having 2 to 30 carbon atoms constituting the chain, in which the number of carbon atoms is preferably 2 to 20, and more preferably 2 to 10. The above alkynyl may include ethynyl, 1-propynyl, 2-propynyl, 1-butynyl, 2-butynyl, 3-butynyl, 1-methylpent-2-ynyl, etc. The term “(C3-C30)cycloalkyl(ene)” is meant to be a mono- or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, etc. The term “(3- to 7-membered)heterocycloalkyl” is meant to be a cycloalkyl having 3 to 7, preferably 5 to 7, ring backbone atoms, and including at least one heteroatom selected from the group consisting of B, N, O, S, Si, and P, and preferably the group consisting of O, S, and N. The above heterocycloalkyl may include tetrahydrofuran, pyrrolidine, thiolan, tetrahydropyran, etc. The term “(C6-C30)aryl(ene)” is meant to be a monocyclic or fused ring radical derived from an aromatic hydrocarbon having 6 to 30 ring backbone carbon atoms, in which the number of the ring backbone carbon atoms is preferably 6 to 25, and more preferably 6 to 18. The above aryl(ene) may be partially saturated, and may comprise a spiro structure. The above aryl may include phenyl, biphenyl, terphenyl, naphthyl, binaphthyl, phenylnaphthyl, naphthylphenyl, phenylterphenyl, fluorenyl, phenylfluorenyl, benzofluorenyl, dibenzofluorenyl, phenanthrenyl, phenylphenanthrenyl, anthracenyl, indenyl, triphenylenyl, pyrenyl, tetracenyl, perylenyl, chrysenyl, naphthacenyl, fluoranthenyl, spirobifluorenyl, azulenyl, etc. More specifically, the above aryl may include phenyl, 1-naphthyl, 2-naphthyl, 1-anthryl, 2-anthryl, 9-anthryl, benzanthryl, 1-phenanthryl, 2-phenanthryl, 3-phenanthryl, 4-phenanthryl, 9-phenanthryl, naphthacenyl, pyrenyl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 1-fluorenyl, 2-fluorenyl, 3-fluorenyl, 4-fluorenyl, 9-fluorenyl, benzo[a]fluorenyl, benzo[b]fluorenyl, benzo[c]fluorenyl, dibenzofluorenyl, 2-biphenylyl, 3-biphenylyl, 4-biphenylyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 3-fluoranthenyl, 4-fluoranthenyl, 8-fluoranthenyl, 9-fluoranthenyl, benzofluoranthenyl, o-tolyl, m-tolyl, p-tolyl, 2,3-xylyl, 3,4-xylyl, 2,5-xylyl, mesityl, o-cumenyl, m-cumenyl, p-cumenyl, p-t-butylphenyl, p-(2-phenylpropyl)phenyl, 4′-methylbiphenylyl, 4″-t-butyl-p-terphenyl-4-yl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a] fluorenyl, 11,11-dimethyl-5-benzo[a] fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a] fluorenyl, 11,11-dimethyl-10-benzo[a] fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b] fluorenyl, 11,11-dimethyl-5-benzo[b] fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b] fluorenyl, 11,11-dimethyl-10-benzo[b] fluorenyl, 11,11-dimethyl-1-benzo[c] fluorenyl, 11,11-dimethyl-2-benzo[c] fluorenyl, 11,11-dimethyl-3-benzo[c]fluorenyl, 11,11-dimethyl-4-benzo[c] fluorenyl, 11,11-dimethyl-5-benzo[c] fluorenyl, 11,11-dimethyl-6-benzo[c] fluorenyl, 11,11-dimethyl-7-benzo[c] fluorenyl, 11,11-dimethyl-8-benzo[c]fluorenyl, 11,11-dimethyl-9-benzo[c] fluorenyl, 11,11-dimethyl-10-benzo[c] fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a] fluorenyl, 11,11-diphenyl-5-benzo[a] fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a] fluorenyl, 11,11-diphenyl-10-benzo[a] fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b] fluorenyl, 11,11-diphenyl-5-benzo[b] fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b] fluorenyl, 11,11-diphenyl-10-benzo[b] fluorenyl, 11,11-diphenyl-1-benzo[c] fluorenyl, 11,11-diphenyl-2-benzo[c] fluorenyl, 11,11-diphenyl-3-benzo[c]fluorenyl, 11,11-diphenyl-4-benzo[c] fluorenyl, 11,11-diphenyl-5-benzo[c] fluorenyl, 11,11-diphenyl-6-benzo[c] fluorenyl, 11,11-diphenyl-7-benzo[c] fluorenyl, 11,11-diphenyl-8-benzo[c]fluorenyl, 11,11-diphenyl-9-benzo[c] fluorenyl, 11,11-diphenyl-10-benzo[c] fluorenyl, etc.

The term “(3- to 30-membered)heteroaryl(ene)” is meant to be an aryl having 3 to 30 ring backbone atoms, and including at least one, preferably 1 to 4 heteroatoms selected from the group consisting of B, N, O, S, Si, and P. The above heteroaryl(ene) may be a monocyclic ring, or a fused ring condensed with at least one benzene ring; may be partially saturated; may be one formed by linking at least one heteroaryl or aryl group to a heteroaryl group via a single bond(s); and may comprise a spiro structure. The above heteroaryl may include a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, and pyridazinyl, and a fused ring-type heteroaryl such as benzofuranyl, benzothiophenyl, isobenzofuranyl, dibenzofuranyl, dibenzothiophenyl, naphthobenzofuranyl, naphthobenzothiophenyl, benzimidazolyl, benzothiazolyl, benzoisothiazolyl, benzoisoxazolyl, benzoxazolyl, isoindolyl, indolyl, benzoindolyl, indazolyl, benzothiadiazolyl, quinolyl, isoquinolyl, cinnolinyl, quinazolinyl, benzoquinazolinyl, quinoxalinyl, benzoquinoxalinyl, naphthyridinyl, carbazolyl, benzocarbazolyl, dibenzocarbazolyl, phenoxazinyl, phenothiazinyl, phenanthridinyl, benzodioxolyl, and dihydroacridinyl. More specifically, the above heteroaryl may include 1-pyrrolyl, 2-pyrrolyl, 3-pyrrolyl, pyrazinyl, 2-pyridinyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolidinyl, 2-indolidinyl, 3-indolidinyl, 5-indolidinyl, 6-indolidinyl, 7-indolidinyl, 8-indolidinyl, 2-imidazopyridinyl, 3-imidazopyridinyl, 5-imidazopyridinyl, 6-imidazopyridinyl, 7-imidazopyridinyl, 8-imidazopyridinyl, 3-pyridinyl, 4-pyridinyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazolyl-1-yl, azacarbazolyl-2-yl, azacarbazolyl-3-yl, azacarbazolyl-4-yl, azacarbazolyl-5-yl, azacarbazolyl-6-yl, azacarbazolyl-7-yl, azacarbazolyl-8-yl, azacarbazolyl-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-t-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-t-butyl-1-indolyl, 4-t-butyl-1-indolyl, 2-t-butyl-3-indolyl, 4-t-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, etc. Furthermore, “halogen” includes F, Cl, Br, and I.

In addition, “ortho (o-),” “meta (m-),” and “para (p-)” are prefixes, which represent the relative positions of substituents respectively. Ortho indicates that two substituents are adjacent to each other, and for example, when two substituents in a benzene derivative occupy positions 1 and 2, it is called an ortho position. Meta indicates that two substituents are at positions 1 and 3, and for example, when two substituents in a benzene derivative occupy positions 1 and 3, it is called a meta position. Para indicates that two substituents are at positions 1 and 4, and for example, when two substituents in a benzene derivative occupy positions 1 and 4, it is called a para position.

Herein, “substituted” in the expression “substituted or unsubstituted” means that a hydrogen atom in a certain functional group is replaced with another atom or another functional group, i.e., a substituent. The substituents of the substituted alkyl(ene), the substituted aryl(ene), the substituted heteroaryl(ene), the substituted cycloalkyl(ene), the substituted cycloalkenyl, the substituted heterocycloalkyl, the substituted alkoxy, the substituted trialkylsilyl, the substituted dialkylarylsilyl, the substituted alkyldiarylsilyl, the substituted triarylsilyl, the substituted mono- or di-alkylamino, the substituted mono- or di-arylamino, and the substituted alkylarylamino in the formulas of the present disclosure, each independently, are at least one selected from the group consisting of deuterium; a halogen; a cyano; a carboxyl; a nitro; a hydroxyl; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 30-membered)heteroaryl unsubstituted or substituted with a (C6-C30)aryl(s); a (C6-C30)aryl unsubstituted or substituted with at least one of a (C1-C30)alkyl(s) and a (3- to 30-membered)heteroaryl(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; an amino; a mono- or di-(C1-C30)alkylamino; a mono- or di-(C6-C30)arylamino unsubstituted or substituted with a (C1-C30)alkyl(s); a (C1-C30)alkyl(C6-C30)arylamino; a (C1-C30)alkylcarbonyl; a (C1-C30)alkoxycarbonyl; a (C6-C30)arylcarbonyl; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl. For example, the substituents may be a substituted or unsubstituted methyl, a substituted or unsubstituted phenyl, a substituted or unsubstituted naphthyl, a substituted or unsubstituted o-biphenyl, a substituted or unsubstituted m-biphenyl, a substituted or unsubstituted p-biphenyl, a substituted or unsubstituted o-terphenyl, a substituted or unsubstituted m-terphenyl, a substituted or unsubstituted p-terphenyl, a substituted or unsubstituted phenanthrenyl, a substituted or unsubstituted chrysenyl, a substituted or unsubstituted fluoranthenyl, a substituted or unsubstituted benzofluorenyl, a substituted or unsubstituted triphenylenyl, a substituted or unsubstituted fluorenyl, a substituted or unsubstituted spirobifluorenyl, a substituted or unsubstituted spiro[benzofluorene-fluorene]yl, a substituted or unsubstituted dibenzothiophenyl, a substituted or unsubstituted dibenzofuranyl, a substituted or unsubstituted benzonaphthothiophenyl, a substituted or unsubstituted benzonaphthofuranyl, a substituted or unsubstituted diphenylamino, a substituted or unsubstituted phenylbiphenylamino, a substituted or unsubstituted phenylterphenylamino, a substituted or unsubstituted naphthylphenylamino, a substituted or unsubstituted naphthylbiphenylamino, a substituted or unsubstituted naphthylterphenylamino, a substituted or unsubstituted naphthylphenanthrenylamino, a substituted or unsubstituted dibiphenylamino, a substituted or unsubstituted difluorenylamino, a substituted or unsubstituted biphenylfluorenylamino, a substituted or unsubstituted diphenyldibenzofuranylamino, etc.

In the formulas of the present disclosure, if a substituent is linked to an adjacent substituent to form a ring or two adjacent substituents are linked to each other to form a ring, the ring may be a substituted or unsubstituted, mono- or polycyclic, (3- to 30-membered) alicyclic or aromatic ring, or the combination thereof, which two or more adjacent substituents are linked to form. In addition, the formed ring may contain at least one heteroatom selected from B, N, O, S, Si, and P, preferably at least one heteroatom selected from N, O, and S. According to one embodiment of the present disclosure, the number of the ring backbone atoms is 5 to 20. According to another embodiment of the present disclosure, the number of the ring backbone atoms is 5 to 15. For example, the fused ring may be a substituted or unsubstituted dibenzothiophene ring, a substituted or unsubstituted dibenzofuran ring, a substituted or unsubstituted naphthalene ring, a substituted or unsubstituted phenanthrene ring, a substituted or unsubstituted fluorene ring, a substituted or unsubstituted benzothiophene ring, a substituted or unsubstituted benzofuran ring, a substituted or unsubstituted indole ring, a substituted or unsubstituted indene ring, a substituted or unsubstituted benzene ring, or a substituted or unsubstituted carbazole ring.

In the formulas of the present disclosure, heteroaryl or heteroarylene may, each independently, contain at least one heteroatom selected from B, N, O, S, Si, and P. In addition, the heteroatom may be bonded to at least one selected from the group consisting of hydrogen, deuterium, a halogen, a cyano, a substituted or unsubstituted (C1-C30)alkyl, a substituted or unsubstituted (C6-C30)aryl, a substituted or unsubstituted (5- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, and a substituted or unsubstituted (C1-C30)alkyl(C6-C30)arylamino.

The plurality of host materials according to one embodiment of the present disclosure comprises a first host material comprising the compound represented by formula 1 and a second host material comprising the compound represented by formula 2, and may be comprised in a light-emitting layer of an organic electroluminescent device according to one embodiment of the present disclosure.

Hereinafter, the compound represented by formula 1 will be described in more detail.

In formula 1, L₁ represents a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene. According to one embodiment of the present disclosure, L₁ represents a single bond, or a substituted or unsubstituted (C6-C15)arylene. According to another embodiment of the present disclosure, L₁ represents a single bond, or an unsubstituted (C6-C15)arylene. Specifically, L₁ may represent a single bond, phenylene, naphthylene, biphenylene, etc.

In formula 1, Ar represents hydrogen, deuterium, a halogen, a cyano, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, a substituted or unsubstituted (3- to 30-membered)heteroaryl, —NR₁R₂, or —SiR₃R₄R₆. According to one embodiment of the present disclosure, Ar represents a substituted or unsubstituted (C6-C30)aryl, or a substituted or unsubstituted (5- to 20-membered)heteroaryl. According to another embodiment of the present disclosure, Ar represents a (C6-C30)aryl unsubstituted or substituted with a (C1-C6)alkyl(s), or an unsubstituted (5- to 20-membered)heteroaryl. Specifically, Ar may represent phenyl, naphthyl, biphenyl, phenanthrenyl, terphenyl, spirobifluorenyl, dimethylfluorenyl, dimethylbenzofluorenyl, dibenzofuranyl, dibenzothiophenyl, benzonaphthofuranyl, benzonaphthothiophenyl, etc.

Herein, R₁ to R₅, each independently, represent a substituted or unsubstituted (C1-C30)alkyl, a substituted or unsubstituted (C6-C30)aryl, or a substituted or unsubstituted (3- to 30-membered)heteroaryl.

In formula 1, X₁ to X₂₅, each independently, represent N or CR₆. According to one embodiment of the present disclosure, X₁ to X₂₅, each independently, may represent CR₆.

Herein, R₆, each independently, represents hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or adjacent R₆'s may be linked to each other to form a ring(s), and if two or more R₆'s are present, each of R₆ may be the same or different. According to one embodiment of the present disclosure, R₆, each independently, represents hydrogen, or a substituted or unsubstituted (C6-C12)aryl; or adjacent R₆'s may be linked to each other to form a ring(s). According to another embodiment of the present disclosure, R₆, each independently, represents hydrogen, or an unsubstituted (C6-C12)aryl; or adjacent R₆'s may be linked to each other to form a ring(s). Specifically, R, each independently, may represent hydrogen, phenyl, etc.; or adjacent R₆'s may be linked to each other to form a benzene ring, etc.

In formula 1, V represents CX₂₆X₂₇, NX₂₈, O, or S. According to one embodiment of the present disclosure, V may represent NX₂₈.

Herein, X₂₆ to X₂₈, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino. According to one embodiment of the present disclosure, X₂₆ to X₂₈, each independently, represent a substituted or unsubstituted (C6-C20)aryl. According to another embodiment of the present disclosure, X₂₆ to X₂₈, each independently, represent a (C6-C20)aryl unsubstituted or substituted with a (C1-C6)alkyl(s) or a (C6-C12)aryl(s). Specifically, X₂ to X₂₈, each independently, may represent phenyl, naphthyl, biphenyl, phenanthrenyl, naphthylphenyl, dimethylfluorenyl, etc.

In formula 1, X₃₁ to X₄₁, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or each of X₃₁ to X₄₁ may be linked to an adjacent substituent of X₃₁ to X₄₁ to form a ring(s). According to one embodiment of the present disclosure, X₃₁ to X₄₁, each independently, represent hydrogen, or a substituted or unsubstituted (C6-C12)aryl; or each of X₃₁ to X₄₁ may be linked to an adjacent substituent of X₃₁ to X₄₁ to form a ring(s). According to another embodiment of the present disclosure, X₃₁ to X₄₁, each independently, represent hydrogen, or an unsubstituted (C6-C12)aryl; or each of X₃₁ to X₄₁ may be linked to an adjacent substituent of X₃₁ to X₄₁ to form a ring(s). Specifically, X₃₁ to X₄₁, each independently, may represent hydrogen, phenyl, etc.; or each of X₃₁ to X₄₁ may be linked to an adjacent substituent of X₃₁ to X₄₁ to form a ring(s).

In formula 1, a, b, c, f, and g, each independently, represent an integer of 1 to 6, and d, e, h, i, j, and k, each independently, represent an integer of 1 to 4, where if a to k are an integer equal to 2 or more, each of X₃₁ to and each of X₄₁ may be the same or different.

In formula 1, * represents a bonding site to L₁.

The compound represented by formula 1 may be at least one selected from the following compounds, but is not limited thereto.

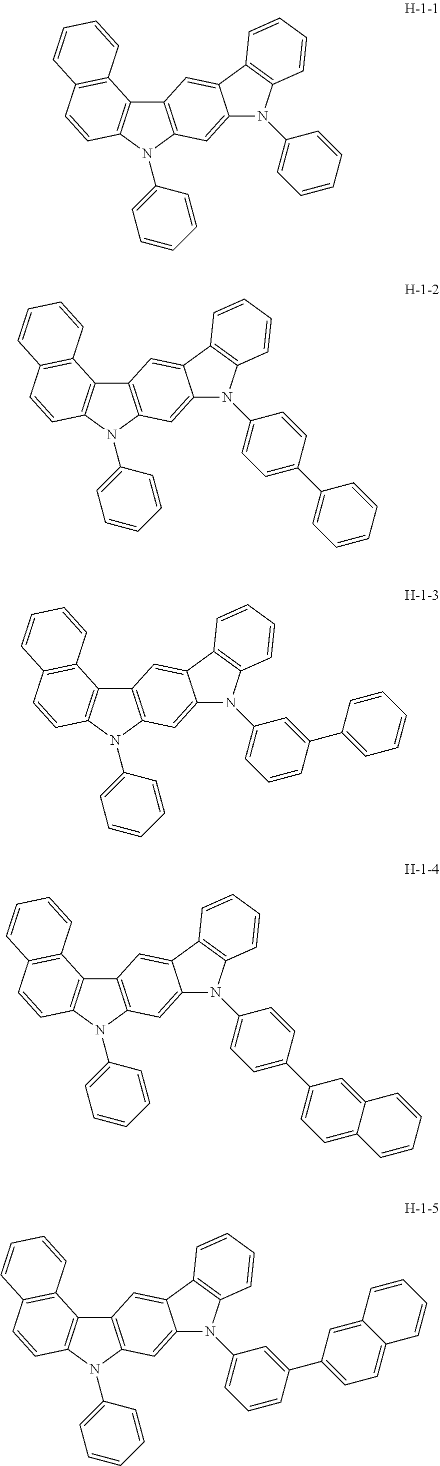 [SMILES: c1ccc(-n2c3ccccc3c3cc4c5c6ccccc6ccc5n(-c5ccccc5)c4cc32)cc1 ; c1ccc(-c2ccc(-n3c4ccccc4c4cc5c6c7ccccc7ccc6n(-c6ccccc6)c5cc43)cc2)cc1 ; c1ccc(-c2cccc(-n3c4ccccc4c4cc5c6c7ccccc7ccc6n(-c6ccccc6)c5cc43)c2)cc1 ; c1ccc(-n2c3cc4c(cc3c3c5ccccc5ccc32)c2ccccc2n4-c2ccc(-c3ccc4ccccc4c3)cc2)cc1 ; c1ccc(-n2c3cc4c(cc3c3c5ccccc5ccc32)c2ccccc2n4-c2cccc(-c3ccc4ccccc4c3)c2)cc1]

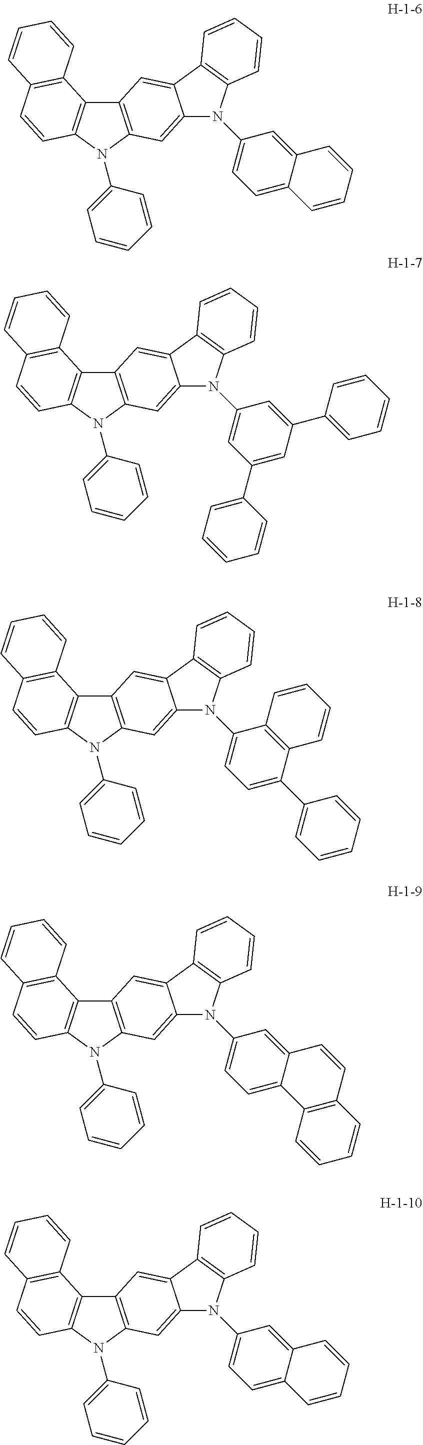 [SMILES: c1ccc(-n2c3cc4c(cc3c3c5ccccc5ccc32)c2ccccc2n4-c2ccc3ccccc3c2)cc1 ; c1ccc(-c2cc(-c3ccccc3)cc(-n3c4ccccc4c4cc5c6c7ccccc7ccc6n(-c6ccccc6)c5cc43)c2)cc1 ; c1ccc(-c2ccc(-n3c4ccccc4c4cc5c6c7ccccc7ccc6n(-c6ccccc6)c5cc43)c3ccccc23)cc1 ; c1ccc(-n2c3cc4c(cc3c3c5ccccc5ccc32)c2ccccc2n4-c2ccc3c(ccc4ccccc43)c2)cc1 ; c1ccc(-n2c3cc4c(cc3c3c5ccccc5ccc32)c2ccccc2n4-c2ccc3ccccc3c2)cc1]

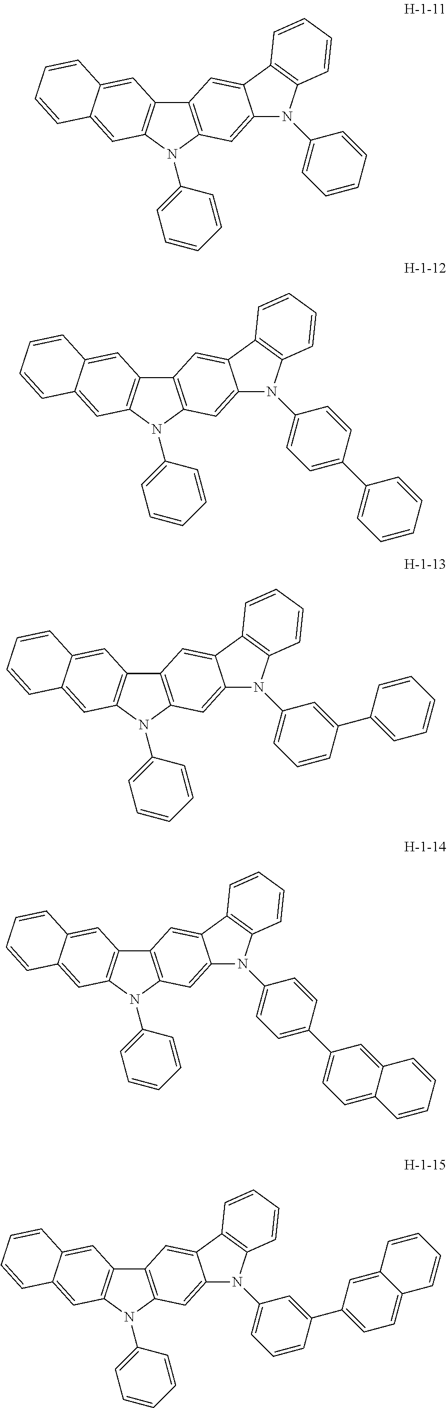 [SMILES: c1ccc(-n2c3ccccc3c3cc4c5cc6ccccc6cc5n(-c5ccccc5)c4cc32)cc1 ; c1ccc(-c2ccc(-n3c4ccccc4c4cc5c6cc7ccccc7cc6n(-c6ccccc6)c5cc43)cc2)cc1 ; c1ccc(-c2cccc(-n3c4ccccc4c4cc5c6cc7ccccc7cc6n(-c6ccccc6)c5cc43)c2)cc1 ; c1ccc(-n2c3cc4ccccc4cc3c3cc4c5ccccc5n(-c5ccc(-c6ccc7ccccc7c6)cc5)c4cc32)cc1 ; c1ccc(-n2c3cc4ccccc4cc3c3cc4c5ccccc5n(-c5cccc(-c6ccc7ccccc7c6)c5)c4cc32)cc1]

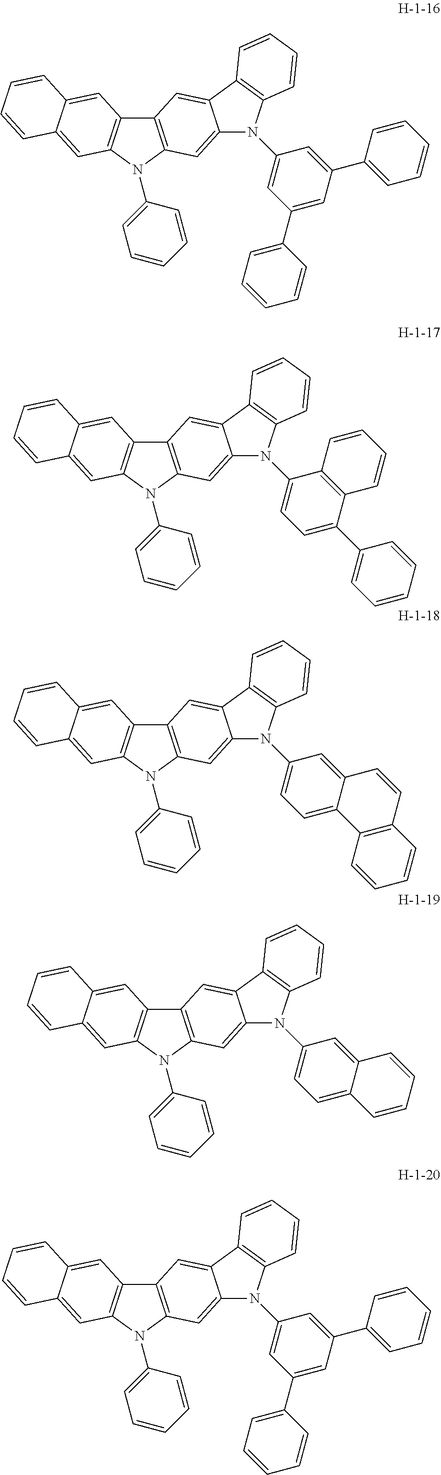 [SMILES: c1ccc(-c2cc(-c3ccccc3)cc(-n3c4ccccc4c4cc5c6cc7ccccc7cc6n(-c6ccccc6)c5cc43)c2)cc1 ; c1ccc(-c2ccc(-n3c4ccccc4c4cc5c6cc7ccccc7cc6n(-c6ccccc6)c5cc43)c3ccccc23)cc1 ; c1ccc(-n2c3cc4ccccc4cc3c3cc4c5ccccc5n(-c5ccc6c(ccc7ccccc76)c5)c4cc32)cc1 ; c1ccc(-n2c3cc4ccccc4cc3c3cc4c5ccccc5n(-c5ccc6ccccc6c5)c4cc32)cc1 ; c1ccc(-c2cc(-c3ccccc3)cc(-n3c4ccccc4c4cc5c6cc7ccccc7cc6n(-c6ccccc6)c5cc43)c2)cc1]

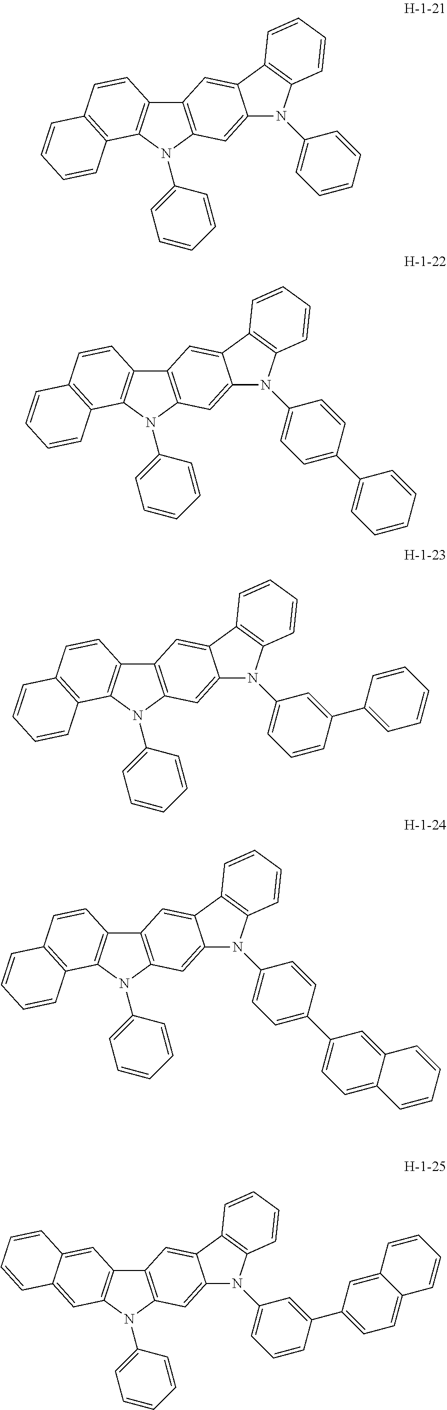 [SMILES: c1ccc(-n2c3ccccc3c3cc4c5ccc6ccccc6c5n(-c5ccccc5)c4cc32)cc1 ; c1ccc(-c2ccc(-n3c4ccccc4c4cc5c6ccc7ccccc7c6n(-c6ccccc6)c5cc43)cc2)cc1 ; c1ccc(-c2cccc(-n3c4ccccc4c4cc5c6ccc7ccccc7c6n(-c6ccccc6)c5cc43)c2)cc1 ; c1ccc(-n2c3cc4c(cc3c3ccc5ccccc5c32)c2ccccc2n4-c2ccc(-c3ccc4ccccc4c3)cc2)cc1 ; c1ccc(-n2c3cc4ccccc4cc3c3cc4c5ccccc5n(-c5cccc(-c6ccc7ccccc7c6)c5)c4cc32)cc1]

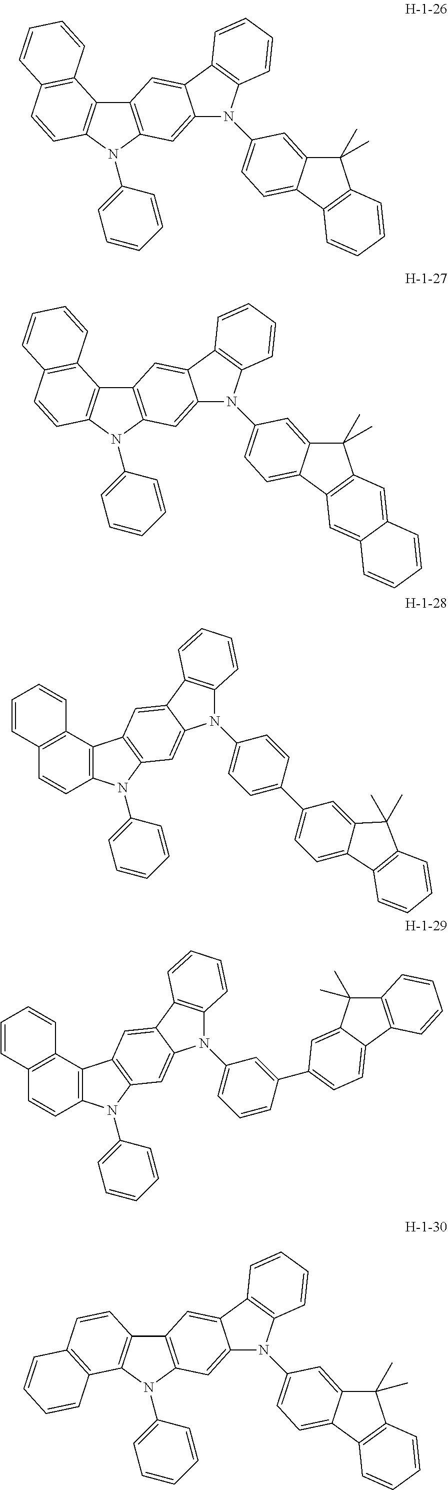 [SMILES: CC1(C)c2ccccc2-c2ccc(-n3c4ccccc4c4cc5c6c7ccccc7ccc6n(-c6ccccc6)c5cc43)cc21 ; CC1(C)c2cc(-n3c4ccccc4c4cc5c6c7ccccc7ccc6n(-c6ccccc6)c5cc43)ccc2-c2cc3ccccc3cc21 ; CC1(C)c2ccccc2-c2ccc(-c3ccc(-n4c5ccccc5c5cc6c7c8ccccc8ccc7n(-c7ccccc7)c6cc54)cc3)cc21 ; CC1(C)c2ccccc2-c2ccc(-c3cccc(-n4c5ccccc5c5cc6c7c8ccccc8ccc7n(-c7ccccc7)c6cc54)c3)cc21 ; CC1(C)c2ccccc2-c2ccc(-n3c4ccccc4c4cc5c6ccc7ccccc7c6n(-c6ccccc6)c5cc43)cc21]

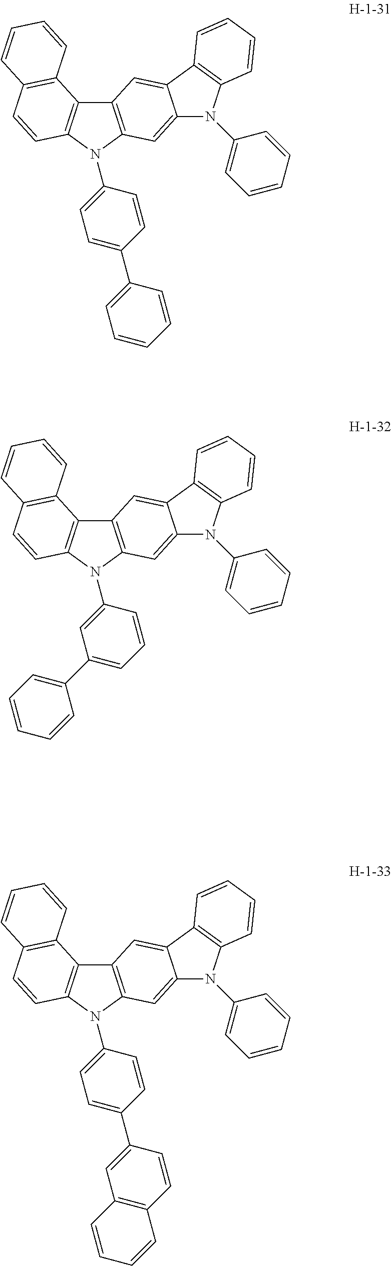 [SMILES: c1ccc(-c2ccc(-n3c4cc5c(cc4c4c6ccccc6ccc43)c3ccccc3n5-c3ccccc3)cc2)cc1 ; c1ccc(-c2cccc(-n3c4cc5c(cc4c4c6ccccc6ccc43)c3ccccc3n5-c3ccccc3)c2)cc1 ; c1ccc(-n2c3ccccc3c3cc4c5c6ccccc6ccc5n(-c5ccc(-c6ccc7ccccc7c6)cc5)c4cc32)cc1]

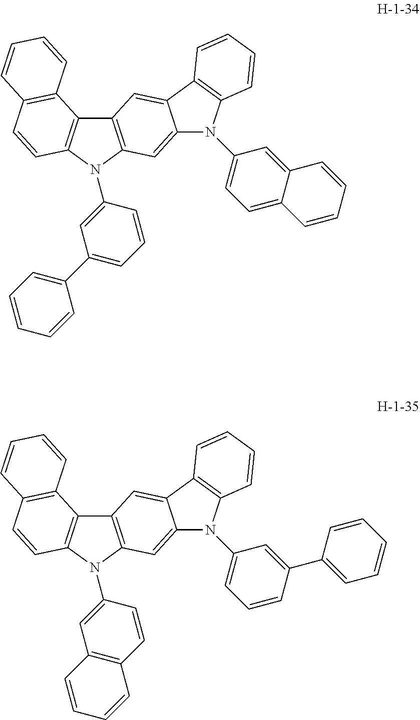 [SMILES: c1ccc(-c2cccc(-n3c4cc5c(cc4c4c6ccccc6ccc43)c3ccccc3n5-c3ccc4ccccc4c3)c2)cc1 ; c1ccc(-c2cccc(-n3c4ccccc4c4cc5c6c7ccccc7ccc6n(-c6ccc7ccccc7c6)c5cc43)c2)cc1]

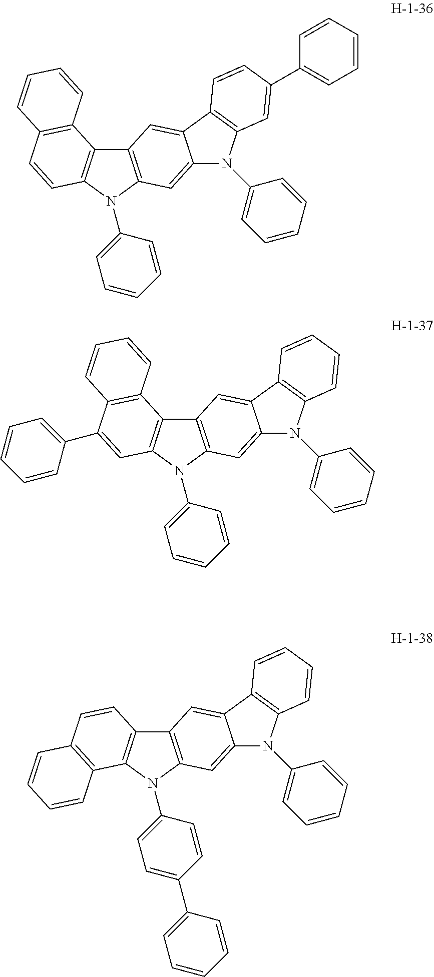

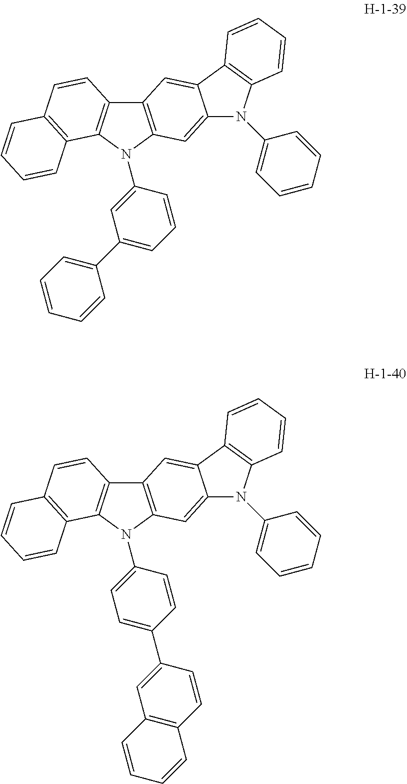 [SMILES: c1ccc(-c2cccc(-n3c4cc5c(cc4c4ccc6ccccc6c43)c3ccccc3n5-c3ccccc3)c2)cc1 ; c1ccc(-n2c3ccccc3c3cc4c5ccc6ccccc6c5n(-c5ccc(-c6ccc7ccccc7c6)cc5)c4cc32)cc1]

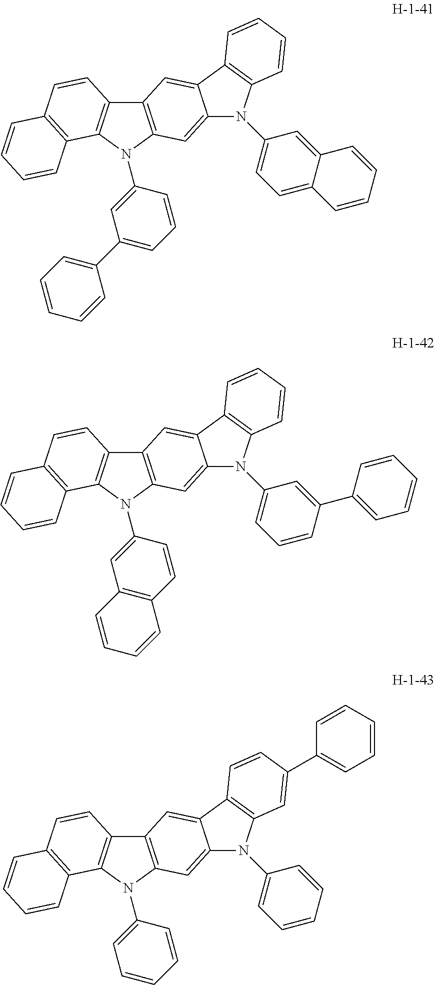 [SMILES: c1ccc(-c2cccc(-n3c4cc5c(cc4c4ccc6ccccc6c43)c3ccccc3n5-c3ccc4ccccc4c3)c2)cc1 ; c1ccc(-c2cccc(-n3c4ccccc4c4cc5c6ccc7ccccc7c6n(-c6ccc7ccccc7c6)c5cc43)c2)cc1 ; c1ccc(-c2ccc3c4cc5c6ccc7ccccc7c6n(-c6ccccc6)c5cc4n(-c4ccccc4)c3c2)cc1]

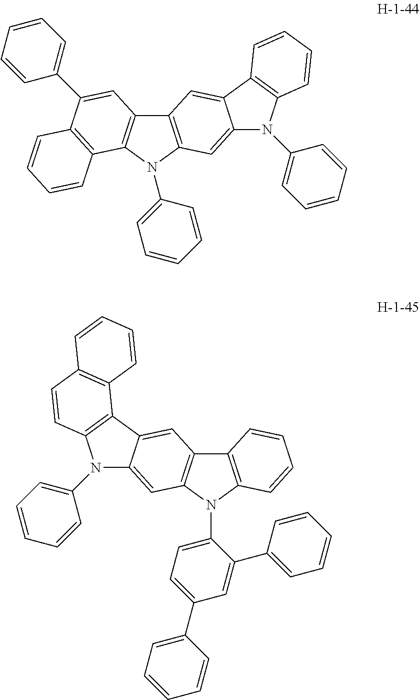 [SMILES: c1ccc(-c2cc3c4cc5c6ccccc6n(-c6ccccc6)c5cc4n(-c4ccccc4)c3c3ccccc23)cc1 ; c1ccc(-c2ccc(-n3c4ccccc4c4cc5c6c7ccccc7ccc6n(-c6ccccc6)c5cc43)c(-c3ccccc3)c2)cc1]

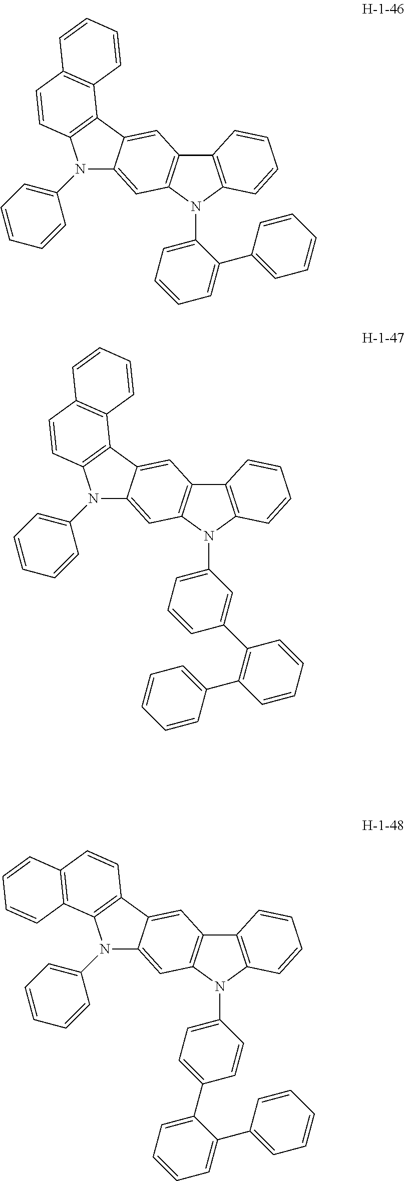 [SMILES: c1ccc(-c2ccccc2-n2c3ccccc3c3cc4c5c6ccccc6ccc5n(-c5ccccc5)c4cc32)cc1 ; c1ccc(-c2ccccc2-c2cccc(-n3c4ccccc4c4cc5c6c7ccccc7ccc6n(-c6ccccc6)c5cc43)c2)cc1 ; c1ccc(-c2ccccc2-c2ccc(-n3c4ccccc4c4cc5c6ccc7ccccc7c6n(-c6ccccc6)c5cc43)cc2)cc1]

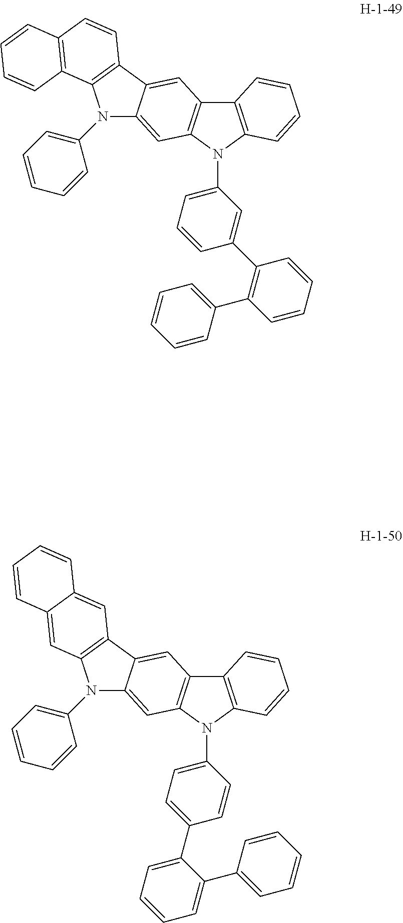 [SMILES: c1ccc(-c2ccccc2-c2cccc(-n3c4ccccc4c4cc5c6ccc7ccccc7c6n(-c6ccccc6)c5cc43)c2)cc1 ; c1ccc(-c2ccccc2-c2ccc(-n3c4ccccc4c4cc5c6cc7ccccc7cc6n(-c6ccccc6)c5cc43)cc2)cc1]

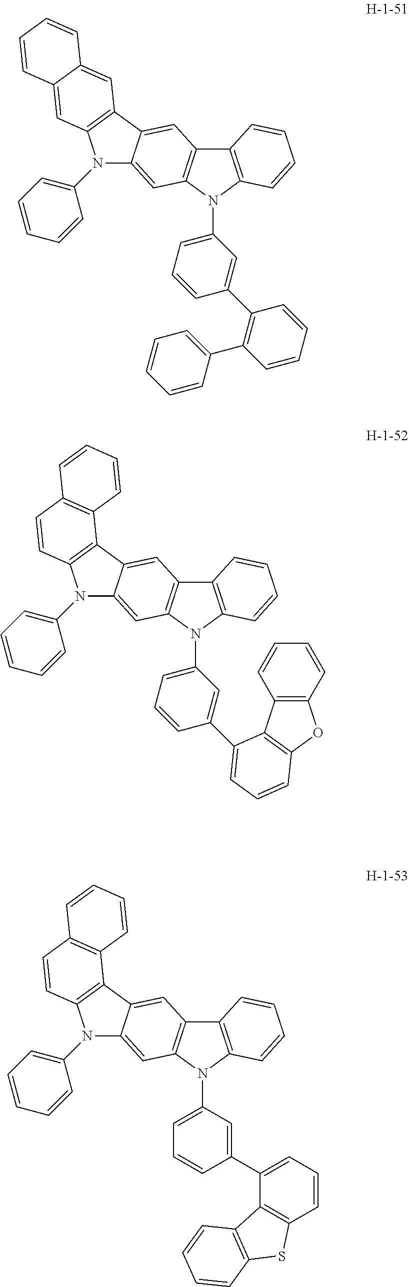 [SMILES: c1ccc(-c2ccccc2-c2cccc(-n3c4ccccc4c4cc5c6cc7ccccc7cc6n(-c6ccccc6)c5cc43)c2)cc1 ; c1ccc(-n2c3cc4c(cc3c3c5ccccc5ccc32)c2ccccc2n4-c2cccc(-c3cccc4oc5ccccc5c34)c2)cc1 ; c1ccc(-n2c3cc4c(cc3c3c5ccccc5ccc32)c2ccccc2n4-c2cccc(-c3cccc4sc5ccccc5c34)c2)cc1]

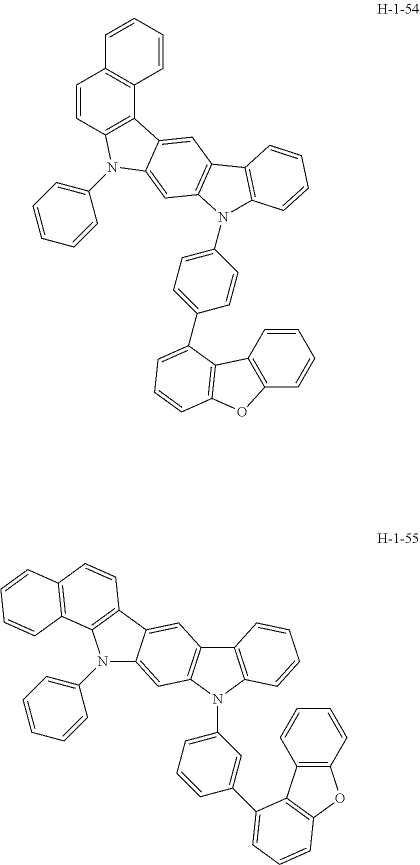 [SMILES: c1ccc(-n2c3cc4c(cc3c3c5ccccc5ccc32)c2ccccc2n4-c2ccc(-c3cccc4oc5ccccc5c34)cc2)cc1 ; c1ccc(-n2c3cc4c(cc3c3ccc5ccccc5c32)c2ccccc2n4-c2cccc(-c3cccc4oc5ccccc5c34)c2)cc1]

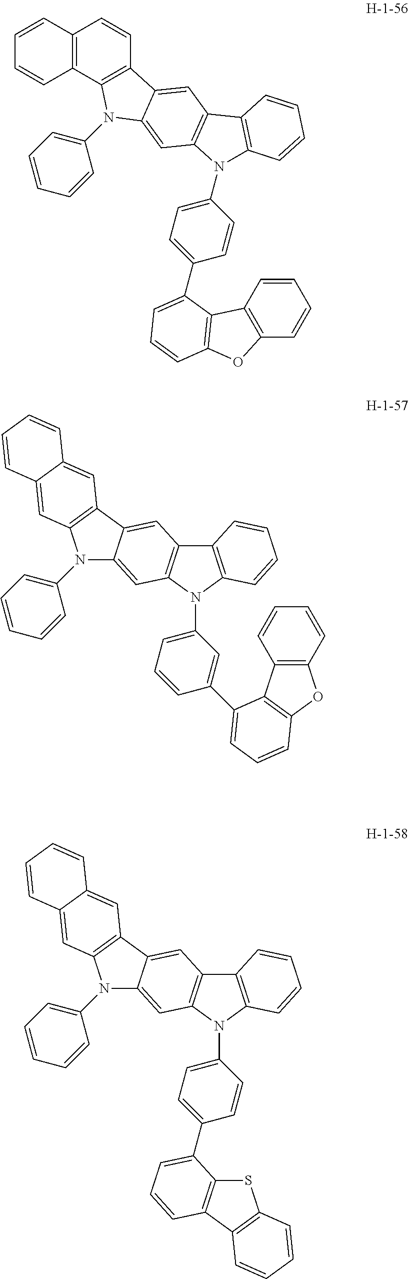 [SMILES: c1ccc(-n2c3cc4c(cc3c3ccc5ccccc5c32)c2ccccc2n4-c2ccc(-c3cccc4oc5ccccc5c34)cc2)cc1 ; c1ccc(-n2c3cc4ccccc4cc3c3cc4c5ccccc5n(-c5cccc(-c6cccc7oc8ccccc8c67)c5)c4cc32)cc1 ; c1ccc(-n2c3cc4ccccc4cc3c3cc4c5ccccc5n(-c5ccc(-c6cccc7c6sc6ccccc67)cc5)c4cc32)cc1]

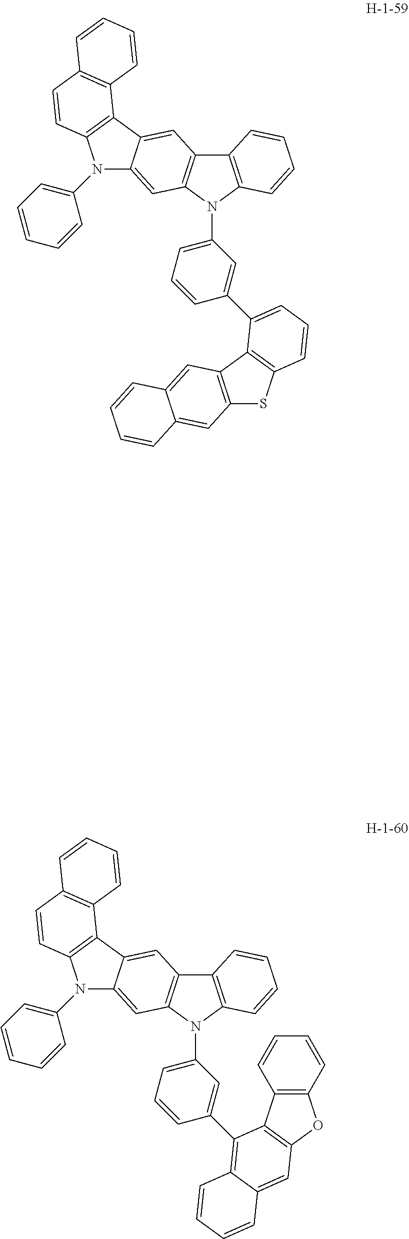 [SMILES: c1ccc(-n2c3cc4c(cc3c3c5ccccc5ccc32)c2ccccc2n4-c2cccc(-c3cccc4sc5cc6ccccc6cc5c34)c2)cc1 ; c1ccc(-n2c3cc4c(cc3c3c5ccccc5ccc32)c2ccccc2n4-c2cccc(-c3c4ccccc4cc4oc5ccccc5c34)c2)cc1]

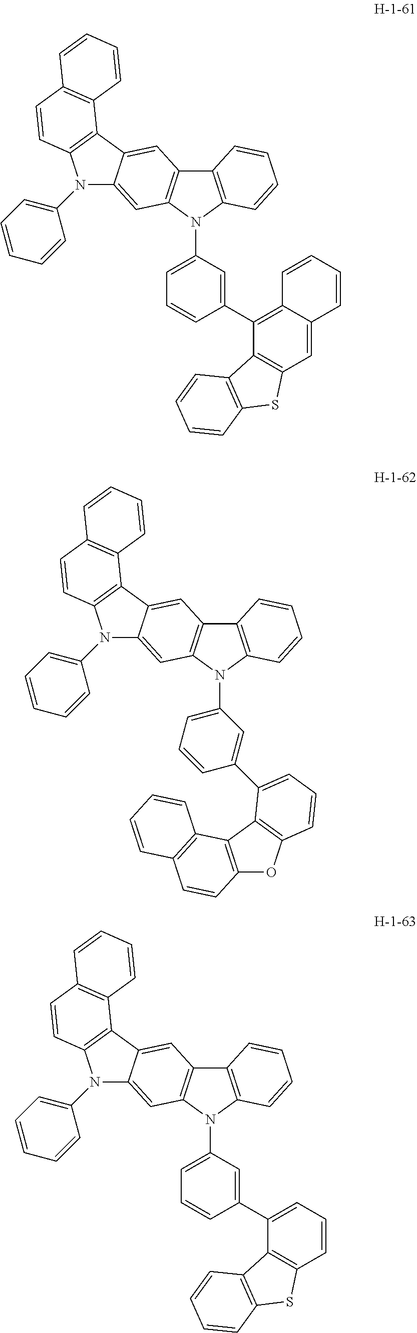 [SMILES: c1ccc(-n2c3cc4c(cc3c3c5ccccc5ccc32)c2ccccc2n4-c2cccc(-c3c4ccccc4cc4sc5ccccc5c34)c2)cc1 ; c1ccc(-n2c3cc4c(cc3c3c5ccccc5ccc32)c2ccccc2n4-c2cccc(-c3cccc4oc5ccc6ccccc6c5c34)c2)cc1 ; c1ccc(-n2c3cc4c(cc3c3c5ccccc5ccc32)c2ccccc2n4-c2cccc(-c3cccc4sc5ccccc5c34)c2)cc1]

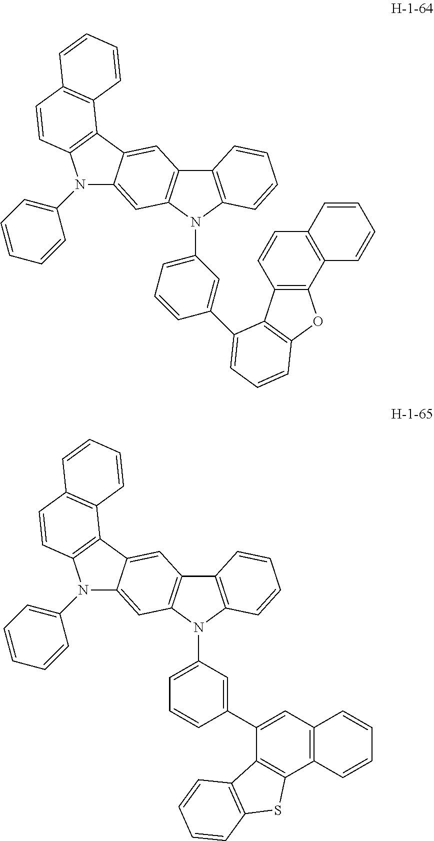 [SMILES: c1ccc(-n2c3cc4c(cc3c3c5ccccc5ccc32)c2ccccc2n4-c2cccc(-c3cccc4oc5c6ccccc6ccc5c34)c2)cc1 ; c1ccc(-n2c3cc4c(cc3c3c5ccccc5ccc32)c2ccccc2n4-c2cccc(-c3cc4ccccc4c4sc5ccccc5c34)c2)cc1]

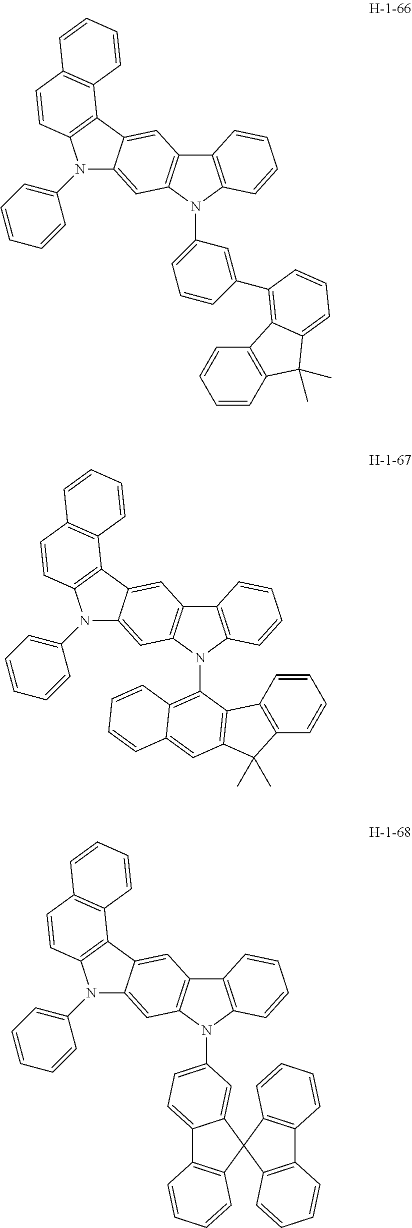 [SMILES: CC1(C)c2ccccc2-c2c(-c3cccc(-n4c5ccccc5c5cc6c7c8ccccc8ccc7n(-c7ccccc7)c6cc54)c3)cccc21 ; CC1(C)c2ccccc2-c2c1cc1ccccc1c2-n1c2ccccc2c2cc3c4c5ccccc5ccc4n(-c4ccccc4)c3cc21 ; c1ccc(-n2c3cc4c(cc3c3c5ccccc5ccc32)c2ccccc2n4-c2ccc3c(c2)C2(c4ccccc4-c4ccccc42)c2ccccc2-3)cc1]

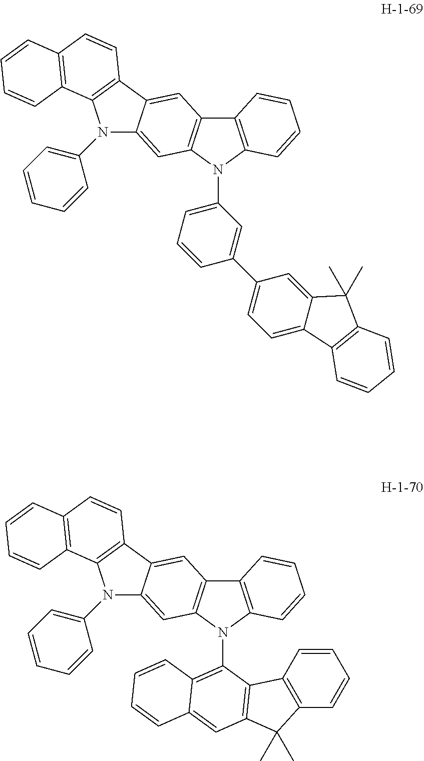 [SMILES: CC1(C)c2ccccc2-c2ccc(-c3cccc(-n4c5ccccc5c5cc6c7ccc8ccccc8c7n(-c7ccccc7)c6cc54)c3)cc21 ; CC1(C)c2ccccc2-c2c1cc1ccccc1c2-n1c2ccccc2c2cc3c4ccc5ccccc5c4n(-c4ccccc4)c3cc21]

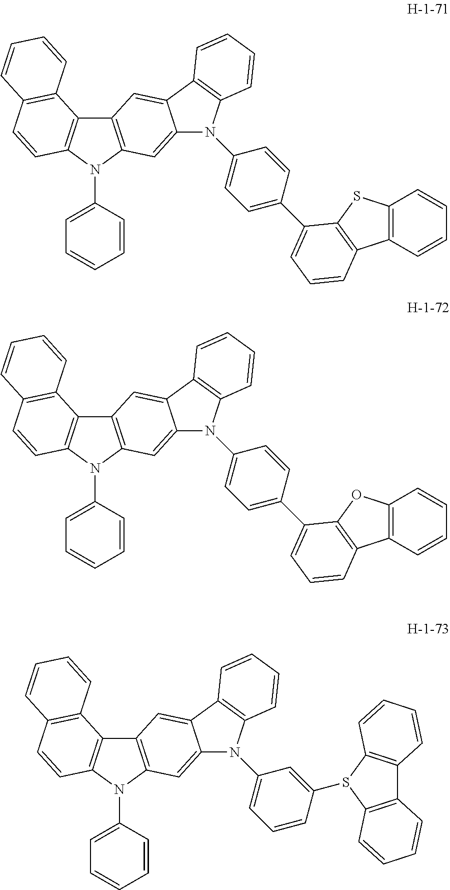

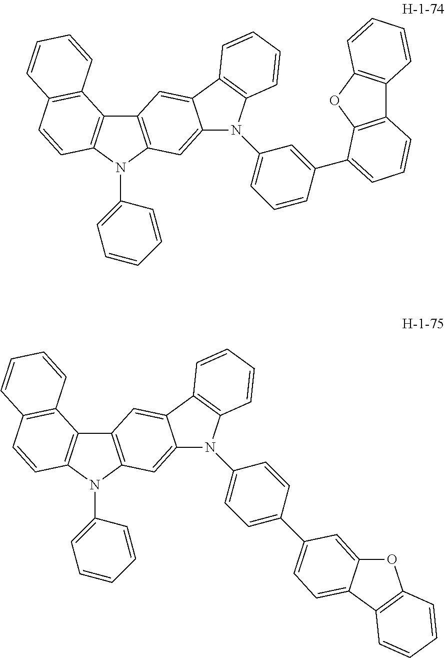 [SMILES: c1ccc(-n2c3cc4c(cc3c3c5ccccc5ccc32)c2ccccc2n4-c2cccc(-c3cccc4c3oc3ccccc34)c2)cc1 ; c1ccc(-n2c3cc4c(cc3c3c5ccccc5ccc32)c2ccccc2n4-c2ccc(-c3ccc4c(c3)oc3ccccc34)cc2)cc1]

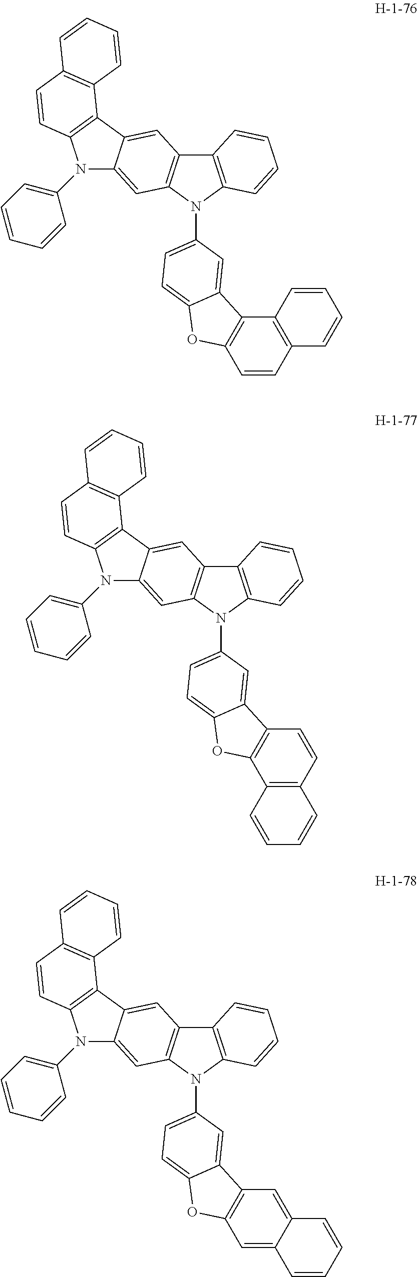 [SMILES: c1ccc(-n2c3cc4c(cc3c3c5ccccc5ccc32)c2ccccc2n4-c2ccc3oc4ccc5ccccc5c4c3c2)cc1 ; c1ccc(-n2c3cc4c(cc3c3c5ccccc5ccc32)c2ccccc2n4-c2ccc3oc4c5ccccc5ccc4c3c2)cc1 ; c1ccc(-n2c3cc4c(cc3c3c5ccccc5ccc32)c2ccccc2n4-c2ccc3oc4cc5ccccc5cc4c3c2)cc1]

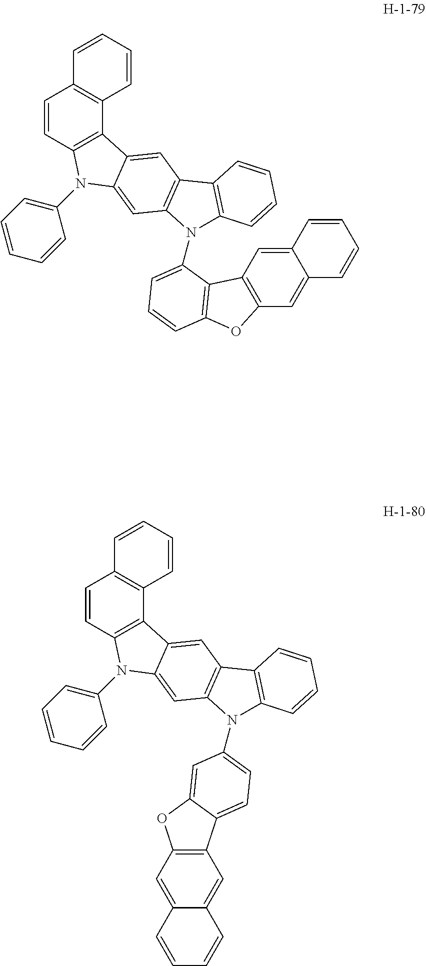 [SMILES: c1ccc(-n2c3cc4c(cc3c3c5ccccc5ccc32)c2ccccc2n4-c2cccc3oc4cc5ccccc5cc4c23)cc1 ; c1ccc(-n2c3cc4c(cc3c3c5ccccc5ccc32)c2ccccc2n4-c2ccc3c(c2)oc2cc4ccccc4cc23)cc1]

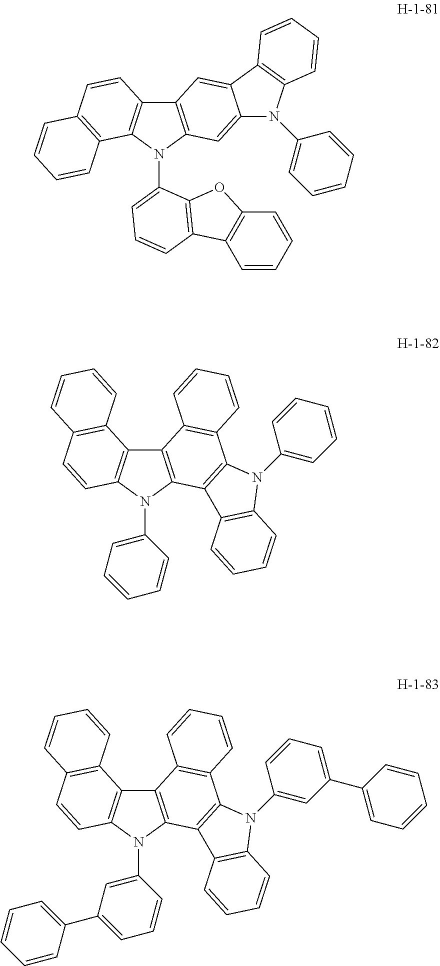 [SMILES: c1ccc(-n2c3ccccc3c3cc4c5ccc6ccccc6c5n(-c5cccc6c5oc5ccccc56)c4cc32)cc1 ; c1ccc(-n2c3ccccc3c3c2c2ccccc2c2c4c5ccccc5ccc4n(-c4ccccc4)c23)cc1 ; c1ccc(-c2cccc(-n3c4ccccc4c4c3c3ccccc3c3c5c6ccccc6ccc5n(-c5cccc(-c6ccccc6)c5)c34)c2)cc1]

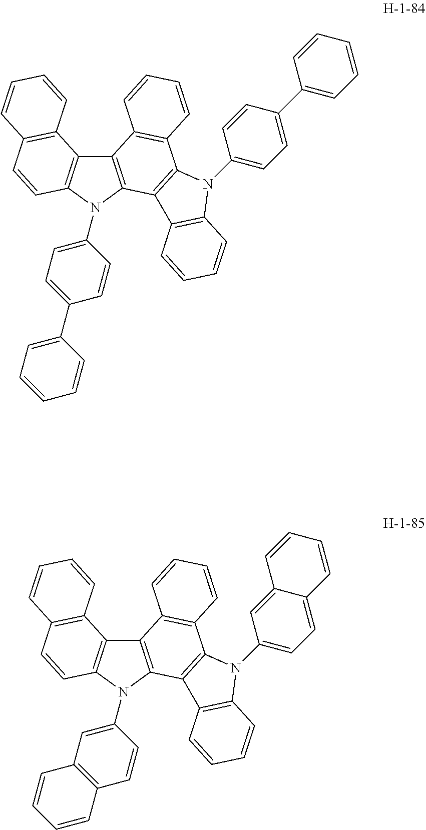 [SMILES: c1ccc(-c2ccc(-n3c4ccccc4c4c3c3ccccc3c3c5c6ccccc6ccc5n(-c5ccc(-c6ccccc6)cc5)c34)cc2)cc1 ; c1ccc2cc(-n3c4ccccc4c4c3c3ccccc3c3c5c6ccccc6ccc5n(-c5ccc6ccccc6c5)c34)ccc2c1]

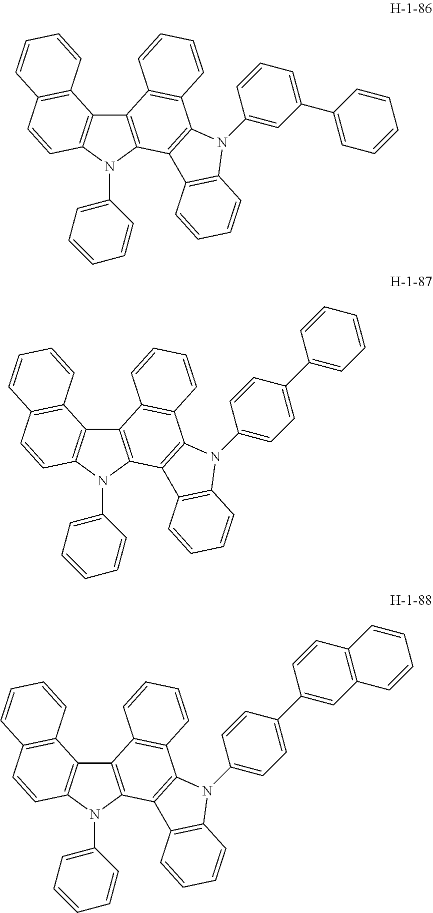 [SMILES: c1ccc(-c2cccc(-n3c4ccccc4c4c3c3ccccc3c3c5c6ccccc6ccc5n(-c5ccccc5)c34)c2)cc1 ; c1ccc(-c2ccc(-n3c4ccccc4c4c3c3ccccc3c3c5c6ccccc6ccc5n(-c5ccccc5)c34)cc2)cc1 ; c1ccc(-n2c3ccc4ccccc4c3c3c4ccccc4c4c(c5ccccc5n4-c4ccc(-c5ccc6ccccc6c5)cc4)c32)cc1]

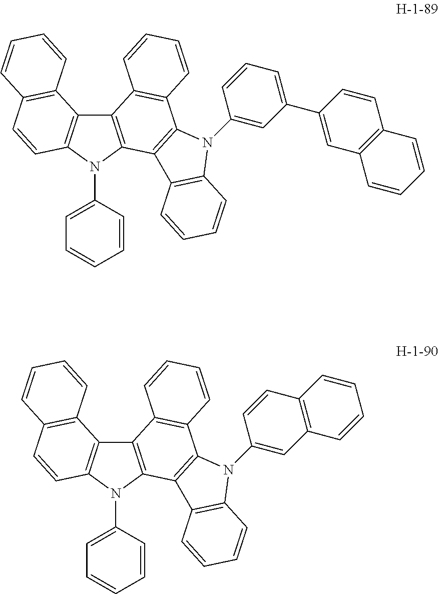 [SMILES: c1ccc(-n2c3ccc4ccccc4c3c3c4ccccc4c4c(c5ccccc5n4-c4cccc(-c5ccc6ccccc6c5)c4)c32)cc1 ; c1ccc(-n2c3ccc4ccccc4c3c3c4ccccc4c4c(c5ccccc5n4-c4ccc5ccccc5c4)c32)cc1]

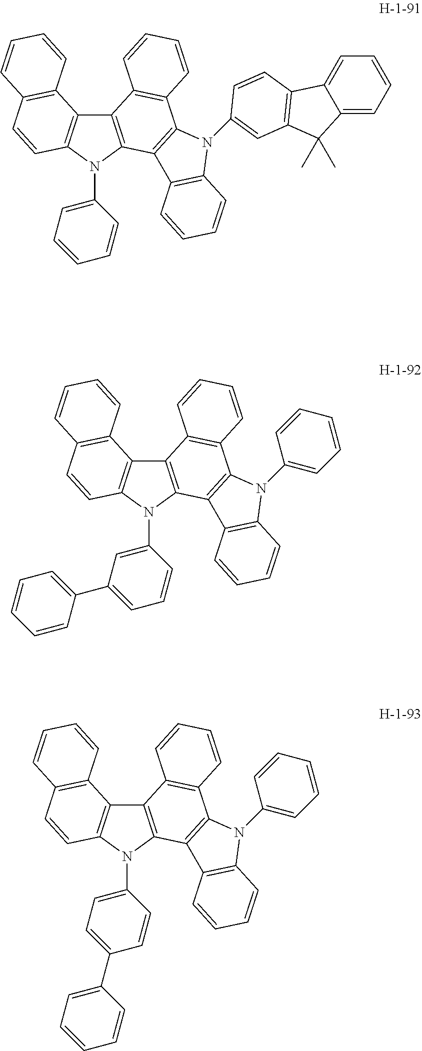 [SMILES: CC1(C)c2ccccc2-c2ccc(-n3c4ccccc4c4c3c3ccccc3c3c5c6ccccc6ccc5n(-c5ccccc5)c34)cc21 ; c1ccc(-c2cccc(-n3c4ccc5ccccc5c4c4c5ccccc5c5c(c6ccccc6n5-c5ccccc5)c43)c2)cc1 ; c1ccc(-c2ccc(-n3c4ccc5ccccc5c4c4c5ccccc5c5c(c6ccccc6n5-c5ccccc5)c43)cc2)cc1]

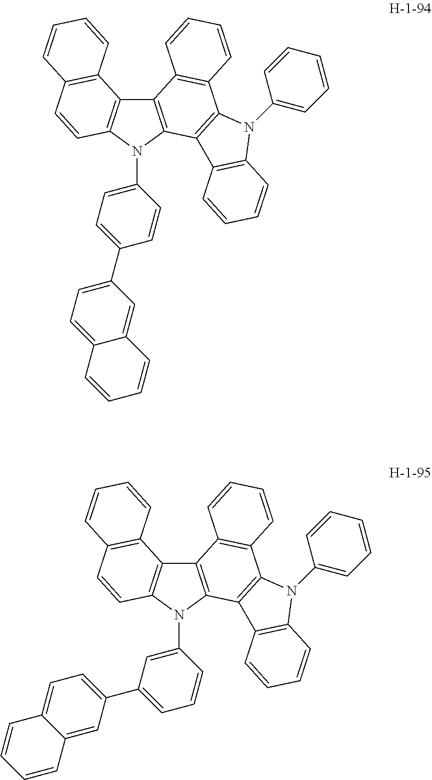 [SMILES: c1ccc(-n2c3ccccc3c3c2c2ccccc2c2c4c5ccccc5ccc4n(-c4ccc(-c5ccc6ccccc6c5)cc4)c23)cc1 ; c1ccc(-n2c3ccccc3c3c2c2ccccc2c2c4c5ccccc5ccc4n(-c4cccc(-c5ccc6ccccc6c5)c4)c23)cc1]

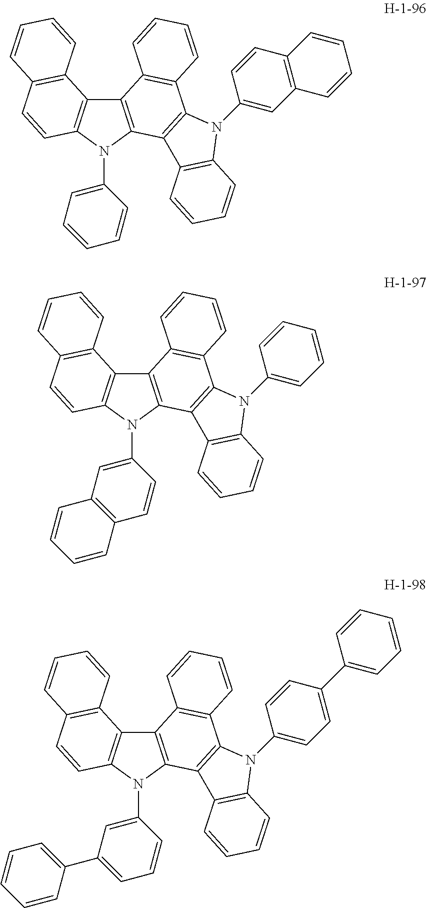

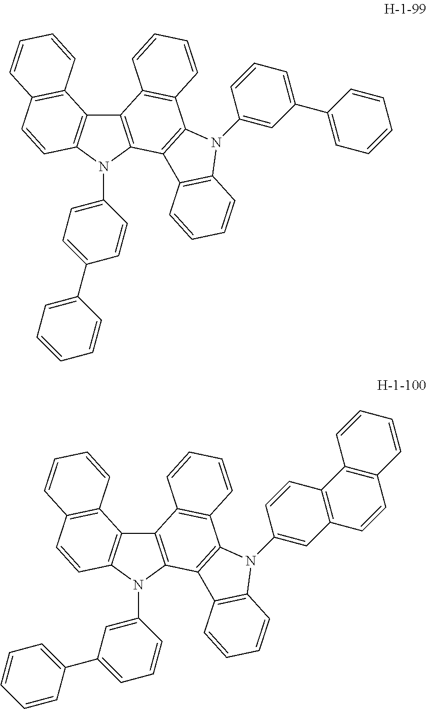 [SMILES: c1ccc(-c2ccc(-n3c4ccc5ccccc5c4c4c5ccccc5c5c(c6ccccc6n5-c5cccc(-c6ccccc6)c5)c43)cc2)cc1 ; c1ccc(-c2cccc(-n3c4ccc5ccccc5c4c4c5ccccc5c5c(c6ccccc6n5-c5ccc6c(ccc7ccccc76)c5)c43)c2)cc1]

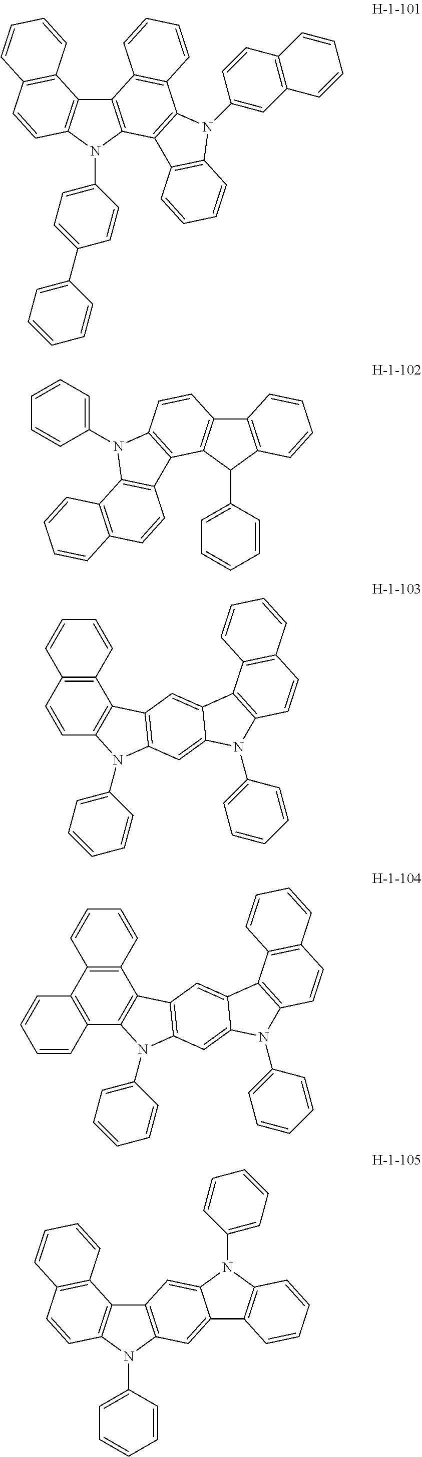

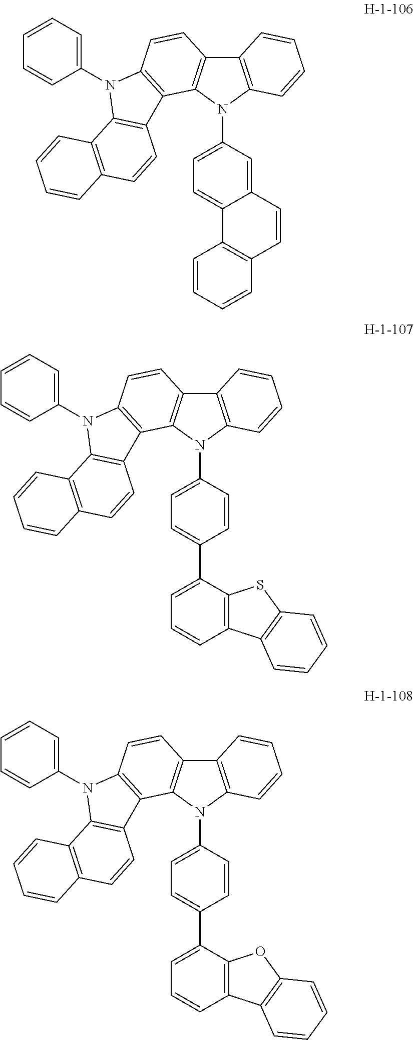 [SMILES: c1ccc(-n2c3ccc4c5ccccc5n(-c5ccc6c(ccc7ccccc76)c5)c4c3c3ccc4ccccc4c32)cc1 ; c1ccc(-n2c3ccc4c5ccccc5n(-c5ccc(-c6cccc7c6sc6ccccc67)cc5)c4c3c3ccc4ccccc4c32)cc1 ; c1ccc(-n2c3ccc4c5ccccc5n(-c5ccc(-c6cccc7c6oc6ccccc67)cc5)c4c3c3ccc4ccccc4c32)cc1]

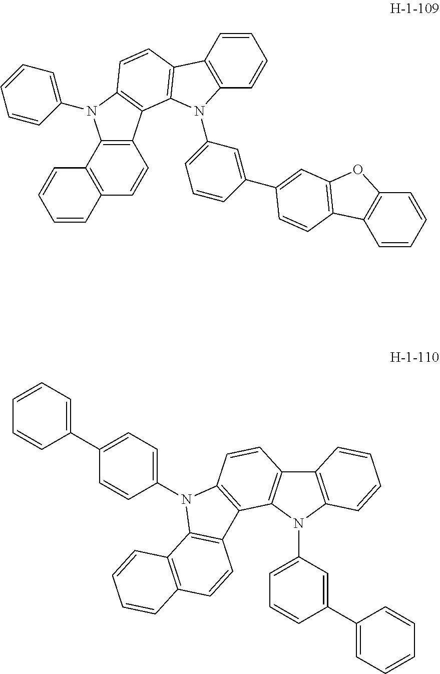 [SMILES: c1ccc(-n2c3ccc4c5ccccc5n(-c5cccc(-c6ccc7c(c6)oc6ccccc67)c5)c4c3c3ccc4ccccc4c32)cc1 ; c1ccc(-c2ccc(-n3c4ccc5c6ccccc6n(-c6cccc(-c7ccccc7)c6)c5c4c4ccc5ccccc5c43)cc2)cc1]

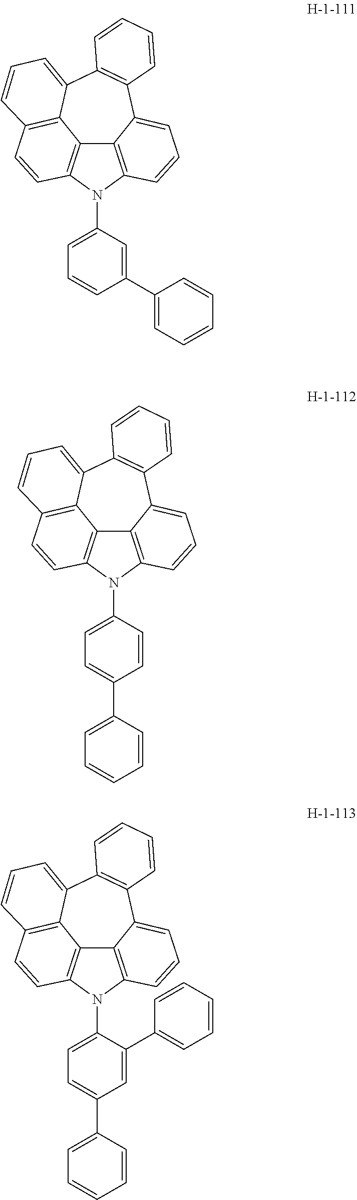

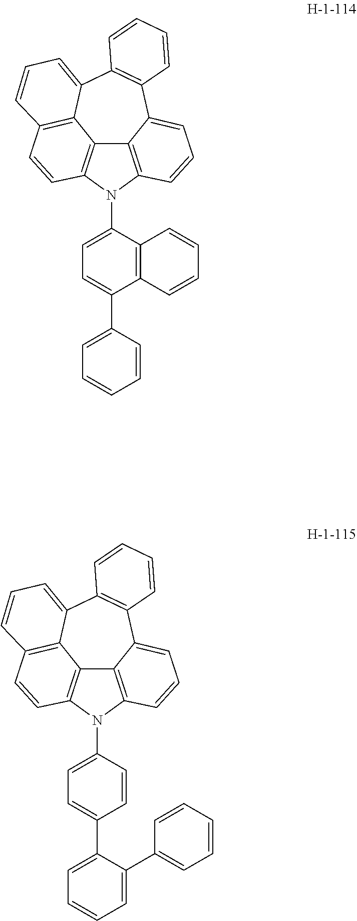 [SMILES: c1ccc(-c2ccc(-n3c4cccc5c4c4c6c(cccc6ccc43)-c3ccccc3-5)c3ccccc23)cc1 ; c1ccc(-c2ccccc2-c2ccc(-n3c4cccc5c4c4c6c(cccc6ccc43)-c3ccccc3-5)cc2)cc1]

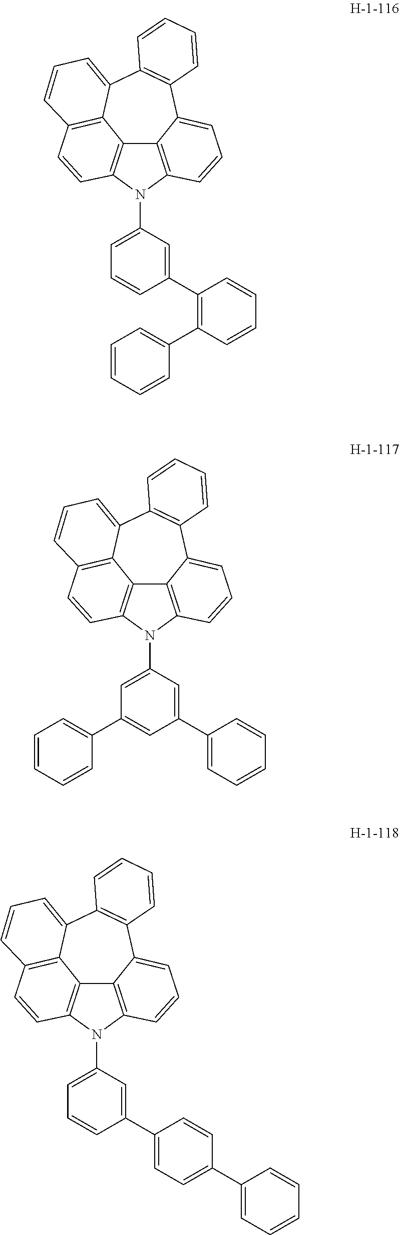 [SMILES: c1ccc(-c2ccccc2-c2cccc(-n3c4cccc5c4c4c6c(cccc6ccc43)-c3ccccc3-5)c2)cc1 ; c1ccc(-c2cc(-c3ccccc3)cc(-n3c4cccc5c4c4c6c(cccc6ccc43)-c3ccccc3-5)c2)cc1 ; c1ccc(-c2ccc(-c3cccc(-n4c5cccc6c5c5c7c(cccc7ccc54)-c4ccccc4-6)c3)cc2)cc1]

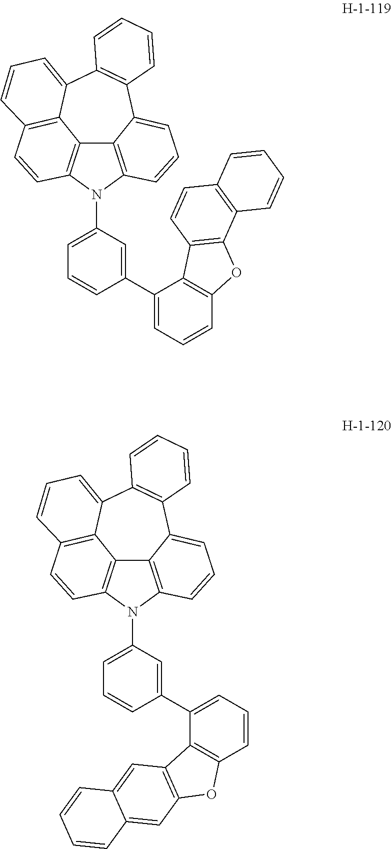 [SMILES: c1cc(-c2cccc3oc4c5ccccc5ccc4c23)cc(-n2c3cccc4c3c3c5c(cccc5ccc32)-c2ccccc2-4)c1 ; c1cc(-c2cccc3oc4cc5ccccc5cc4c23)cc(-n2c3cccc4c3c3c5c(cccc5ccc32)-c2ccccc2-4)c1]

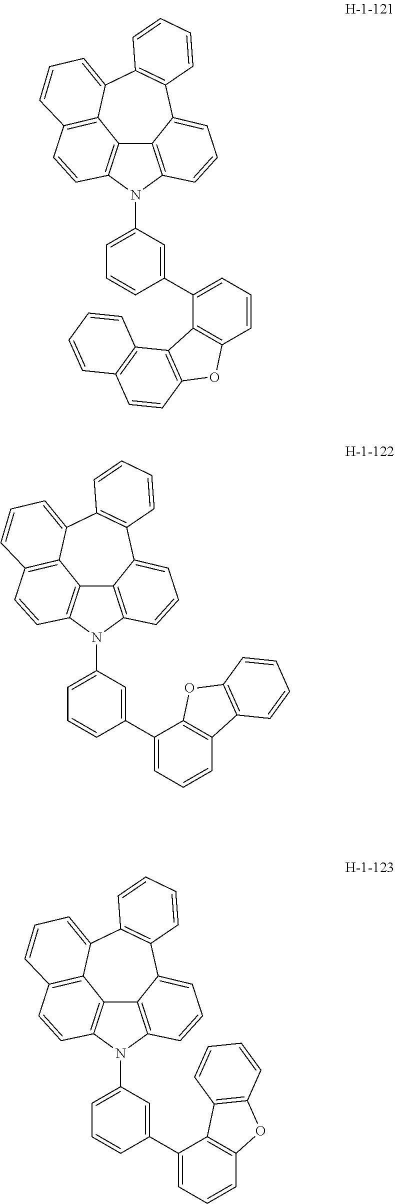 [SMILES: c1cc(-c2cccc3oc4ccc5ccccc5c4c23)cc(-n2c3cccc4c3c3c5c(cccc5ccc32)-c2ccccc2-4)c1 ; c1cc(-c2cccc3c2oc2ccccc23)cc(-n2c3cccc4c3c3c5c(cccc5ccc32)-c2ccccc2-4)c1 ; c1cc(-c2cccc3oc4ccccc4c23)cc(-n2c3cccc4c3c3c5c(cccc5ccc32)-c2ccccc2-4)c1]

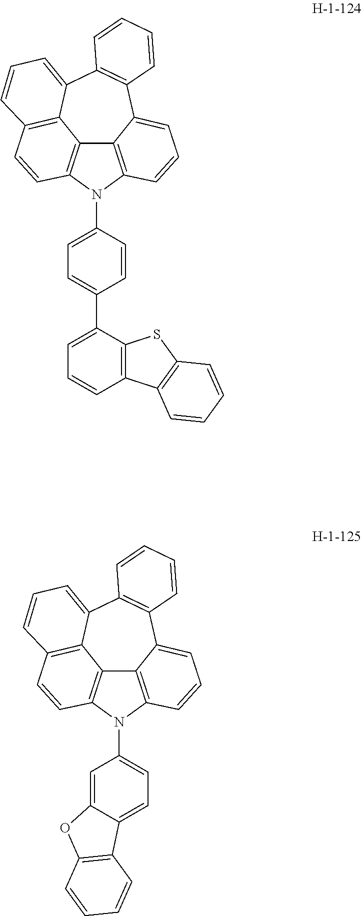 [SMILES: c1ccc2c(c1)-c1cccc3ccc4c(c13)c1c-2cccc1n4-c1ccc(-c2cccc3c2sc2ccccc23)cc1 ; c1ccc2c(c1)-c1cccc3ccc4c(c13)c1c-2cccc1n4-c1ccc2c(c1)oc1ccccc12]

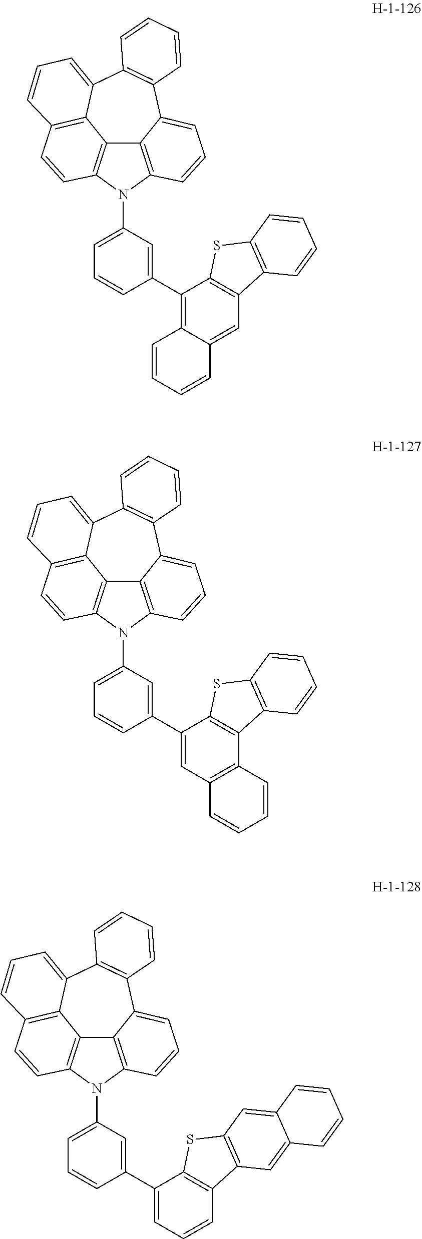 [SMILES: c1cc(-c2c3ccccc3cc3c2sc2ccccc23)cc(-n2c3cccc4c3c3c5c(cccc5ccc32)-c2ccccc2-4)c1 ; c1cc(-c2cc3ccccc3c3c2sc2ccccc23)cc(-n2c3cccc4c3c3c5c(cccc5ccc32)-c2ccccc2-4)c1 ; c1cc(-c2cccc3c2sc2cc4ccccc4cc23)cc(-n2c3cccc4c3c3c5c(cccc5ccc32)-c2ccccc2-4)c1]

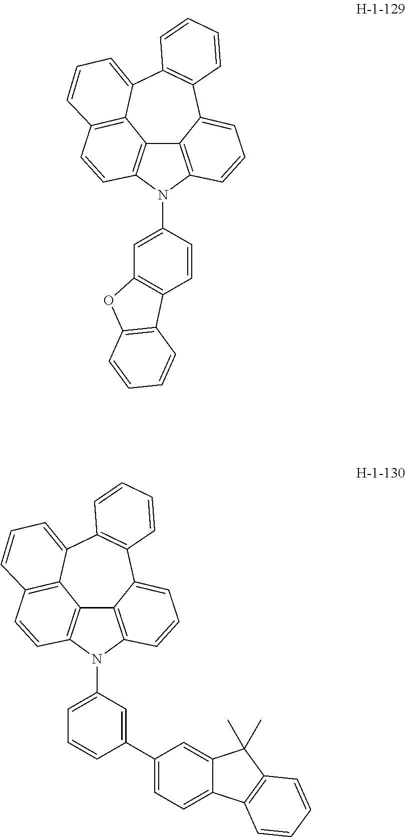 [SMILES: c1ccc2c(c1)-c1cccc3ccc4c(c13)c1c-2cccc1n4-c1ccc2c(c1)oc1ccccc12 ; CC1(C)c2ccccc2-c2ccc(-c3cccc(-n4c5cccc6c5c5c7c(cccc7ccc54)-c4ccccc4-6)c3)cc21]

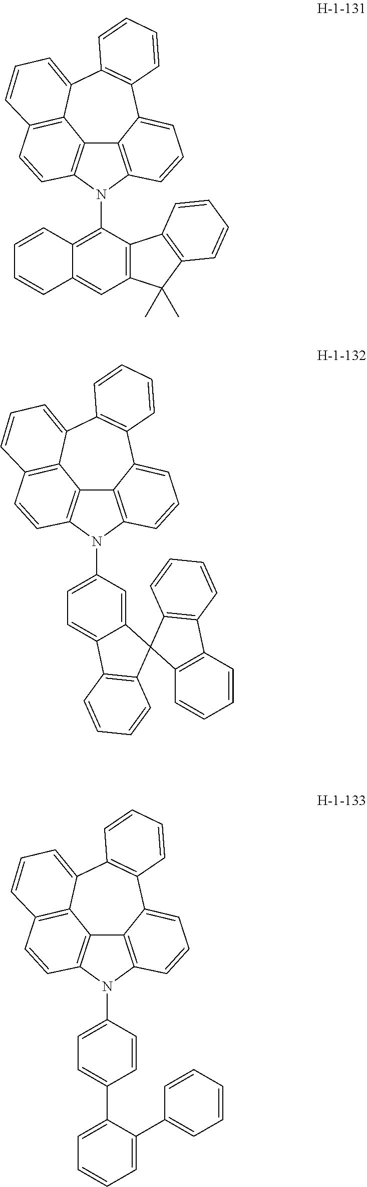 [SMILES: CC1(C)c2ccccc2-c2c1cc1ccccc1c2-n1c2cccc3c2c2c4c(cccc4ccc21)-c1ccccc1-3 ; c1ccc2c(c1)-c1cccc3ccc4c(c13)c1c-2cccc1n4-c1ccc2c(c1)C1(c3ccccc3-c3ccccc31)c1ccccc1-2 ; c1ccc(-c2ccccc2-c2ccc(-n3c4cccc5c4c4c6c(cccc6ccc43)-c3ccccc3-5)cc2)cc1]

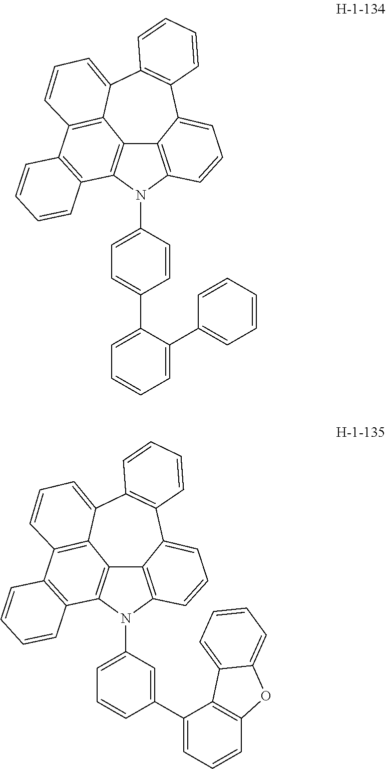 [SMILES: c1ccc(-c2ccccc2-c2ccc(-n3c4cccc5c4c4c6c(cccc6c6ccccc6c43)-c3ccccc3-5)cc2)cc1 ; c1cc(-c2cccc3oc4ccccc4c23)cc(-n2c3cccc4c3c3c5c(cccc5c5ccccc5c32)-c2ccccc2-4)c1]

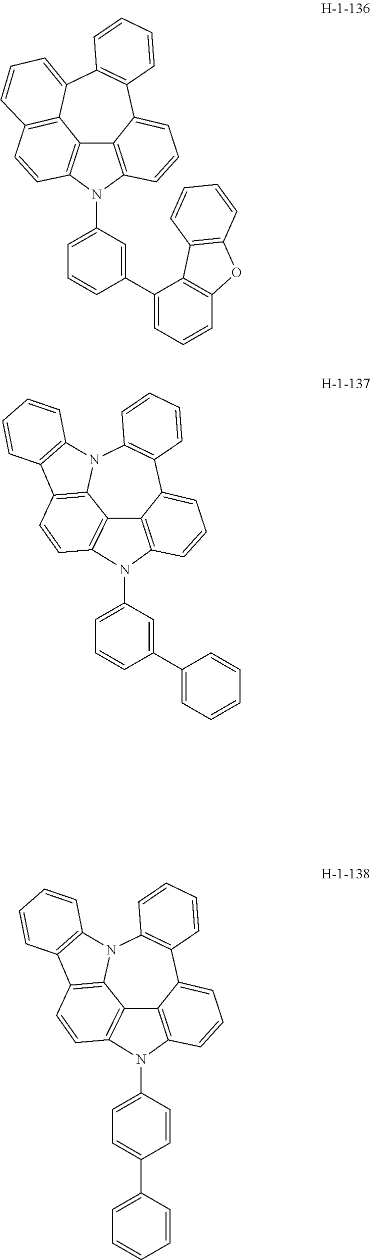 [SMILES: c1cc(-c2cccc3oc4ccccc4c23)cc(-n2c3cccc4c3c3c5c(cccc5ccc32)-c2ccccc2-4)c1 ; c1ccc(-c2cccc(-n3c4cccc5c6ccccc6n6c7ccccc7c7ccc3c(c54)c76)c2)cc1 ; c1ccc(-c2ccc(-n3c4cccc5c6ccccc6n6c7ccccc7c7ccc3c(c54)c76)cc2)cc1]

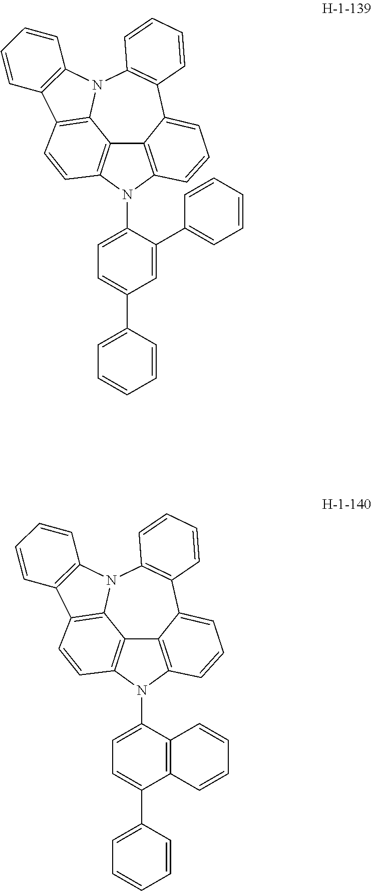 [SMILES: c1ccc(-c2ccc(-n3c4cccc5c6ccccc6n6c7ccccc7c7ccc3c(c54)c76)c(-c3ccccc3)c2)cc1 ; c1ccc(-c2ccc(-n3c4cccc5c6ccccc6n6c7ccccc7c7ccc3c(c54)c76)c3ccccc23)cc1]

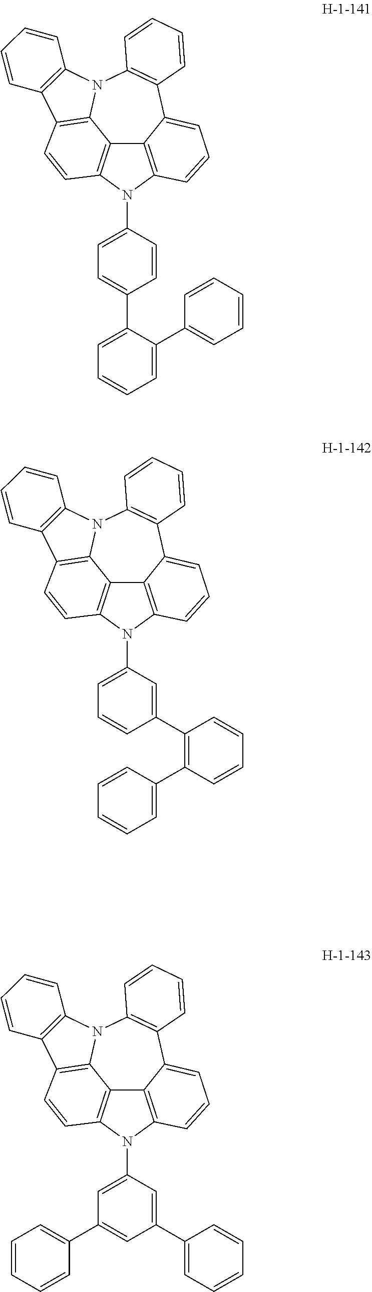 [SMILES: c1ccc(-c2ccccc2-c2ccc(-n3c4cccc5c6ccccc6n6c7ccccc7c7ccc3c(c54)c76)cc2)cc1 ; c1ccc(-c2ccccc2-c2cccc(-n3c4cccc5c6ccccc6n6c7ccccc7c7ccc3c(c54)c76)c2)cc1 ; c1ccc(-c2cc(-c3ccccc3)cc(-n3c4cccc5c6ccccc6n6c7ccccc7c7ccc3c(c54)c76)c2)cc1]

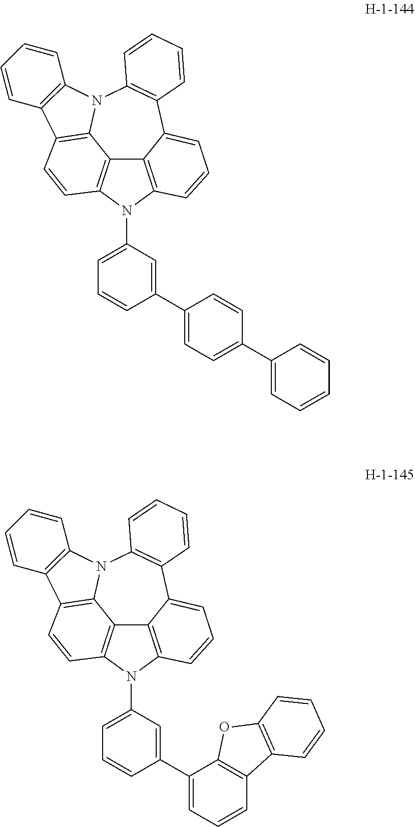 [SMILES: c1ccc(-c2ccc(-c3cccc(-n4c5cccc6c7ccccc7n7c8ccccc8c8ccc4c(c65)c87)c3)cc2)cc1 ; c1cc(-c2cccc3c2oc2ccccc23)cc(-n2c3cccc4c5ccccc5n5c6ccccc6c6ccc2c(c43)c65)c1]

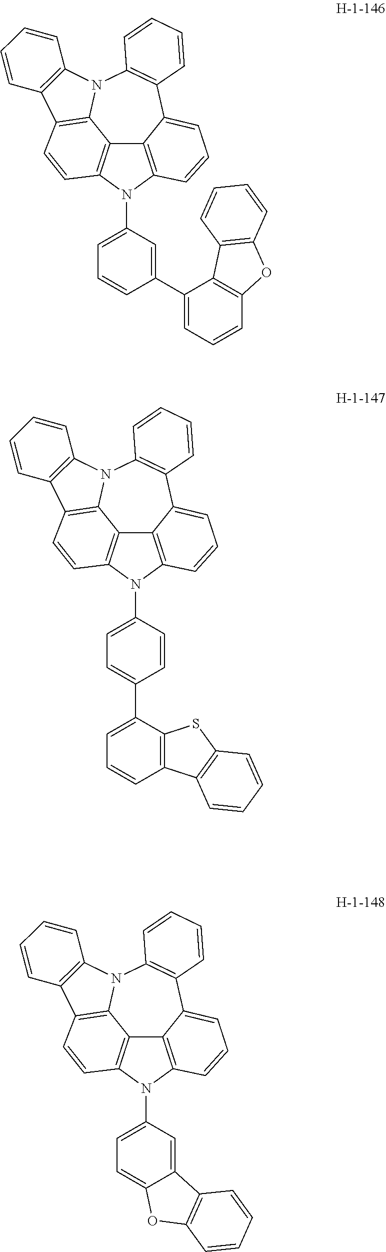 [SMILES: c1cc(-c2cccc3oc4ccccc4c23)cc(-n2c3cccc4c5ccccc5n5c6ccccc6c6ccc2c(c43)c65)c1 ; c1ccc2c(c1)sc1c(-c3ccc(-n4c5cccc6c7ccccc7n7c8ccccc8c8ccc4c(c65)c87)cc3)cccc12 ; c1ccc2c(c1)oc1ccc(-n3c4cccc5c6ccccc6n6c7ccccc7c7ccc3c(c54)c76)cc12]

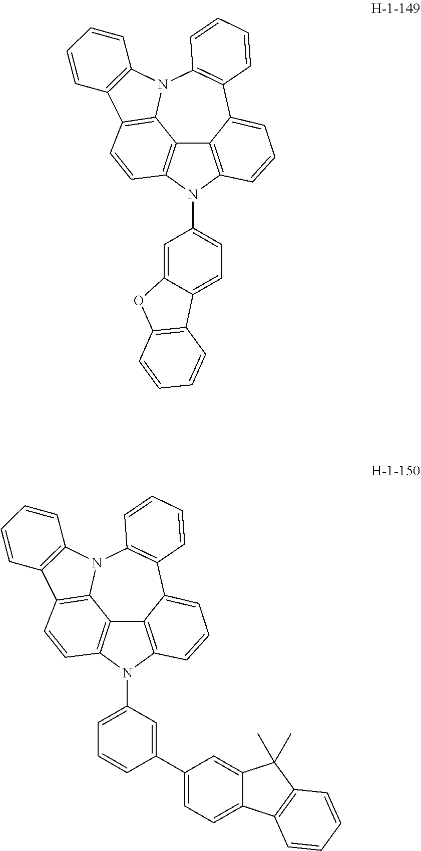 [SMILES: c1ccc2c(c1)oc1cc(-n3c4cccc5c6ccccc6n6c7ccccc7c7ccc3c(c54)c76)ccc12 ; CC1(C)c2ccccc2-c2ccc(-c3cccc(-n4c5cccc6c7ccccc7n7c8ccccc8c8ccc4c(c65)c87)c3)cc21]

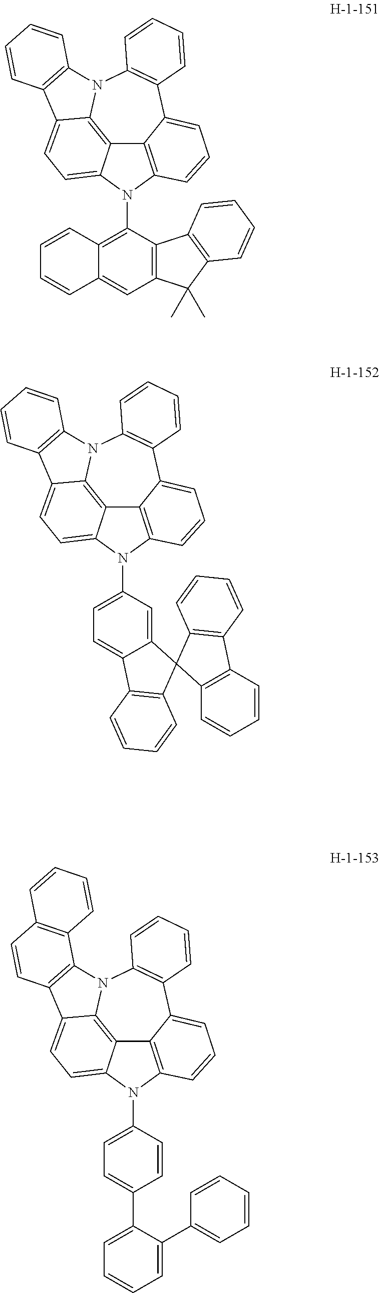

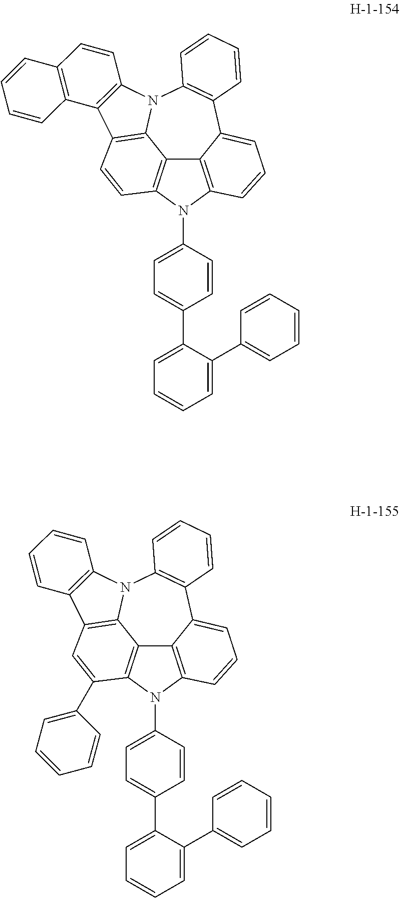 [SMILES: c1ccc(-c2ccccc2-c2ccc(-n3c4cccc5c6ccccc6n6c7ccc8ccccc8c7c7ccc3c(c54)c76)cc2)cc1 ; c1ccc(-c2ccccc2-c2ccc(-n3c4cccc5c6ccccc6n6c7ccccc7c7cc(-c8ccccc8)c3c(c54)c76)cc2)cc1]

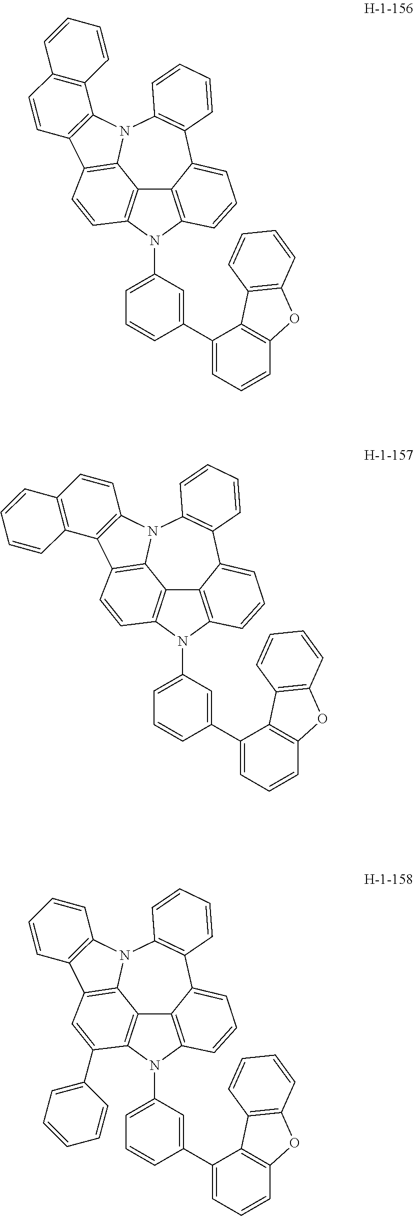 [SMILES: c1cc(-c2cccc3oc4ccccc4c23)cc(-n2c3cccc4c5ccccc5n5c6c7ccccc7ccc6c6ccc2c(c43)c65)c1 ; c1cc(-c2cccc3oc4ccccc4c23)cc(-n2c3cccc4c5ccccc5n5c6ccc7ccccc7c6c6ccc2c(c43)c65)c1 ; c1ccc(-c2cc3c4ccccc4n4c5ccccc5c5cccc6c5c(c2n6-c2cccc(-c5cccc6oc7ccccc7c56)c2)c34)cc1]

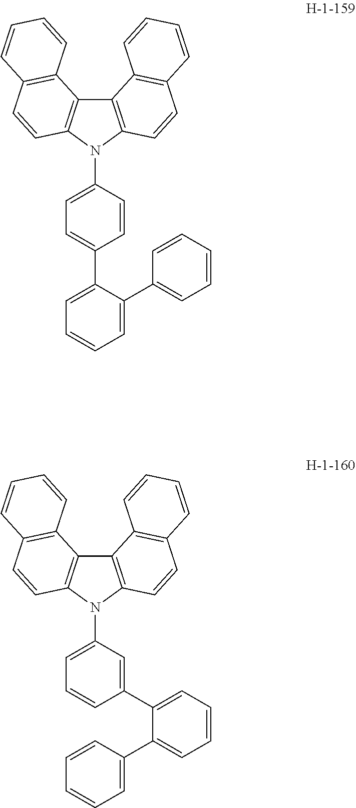 [SMILES: c1ccc(-c2ccccc2-c2ccc(-n3c4ccc5ccccc5c4c4c5ccccc5ccc43)cc2)cc1 ; c1ccc(-c2ccccc2-c2cccc(-n3c4ccc5ccccc5c4c4c5ccccc5ccc43)c2)cc1]

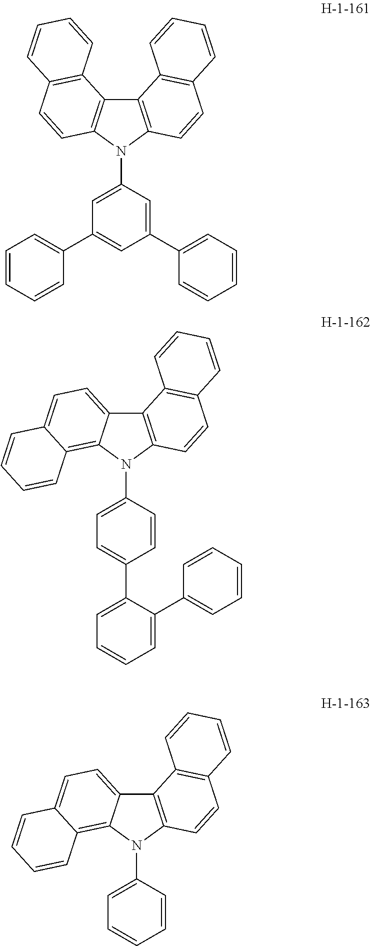

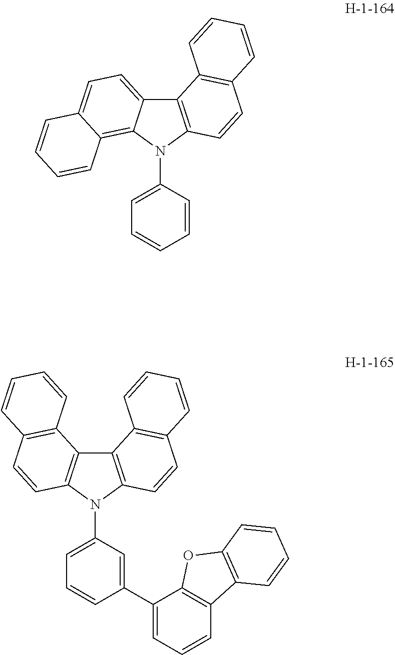 [SMILES: c1ccc(-n2c3ccc4ccccc4c3c3ccc4ccccc4c32)cc1 ; c1cc(-c2cccc3c2oc2ccccc23)cc(-n2c3ccc4ccccc4c3c3c4ccccc4ccc32)c1]

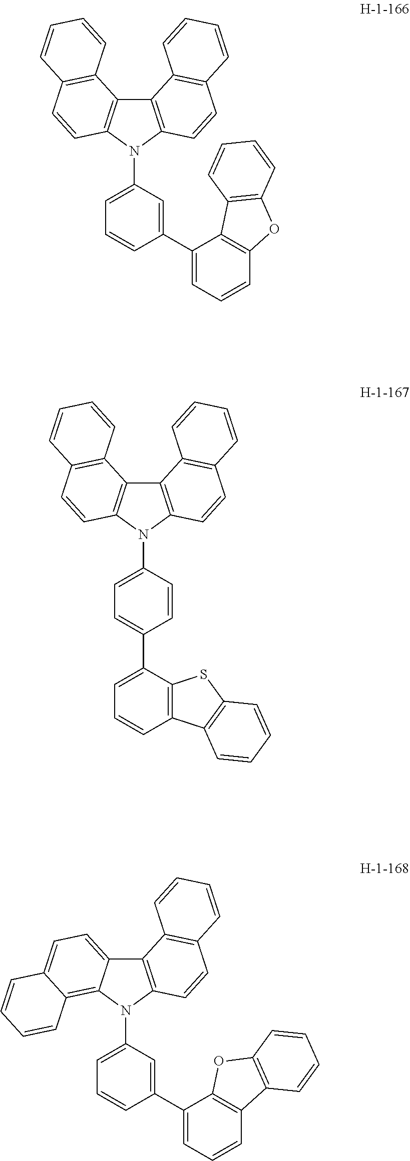 [SMILES: c1cc(-c2cccc3oc4ccccc4c23)cc(-n2c3ccc4ccccc4c3c3c4ccccc4ccc32)c1 ; c1ccc2c(c1)ccc1c2c2c3ccccc3ccc2n1-c1ccc(-c2cccc3c2sc2ccccc23)cc1 ; c1cc(-c2cccc3c2oc2ccccc23)cc(-n2c3ccc4ccccc4c3c3ccc4ccccc4c32)c1]

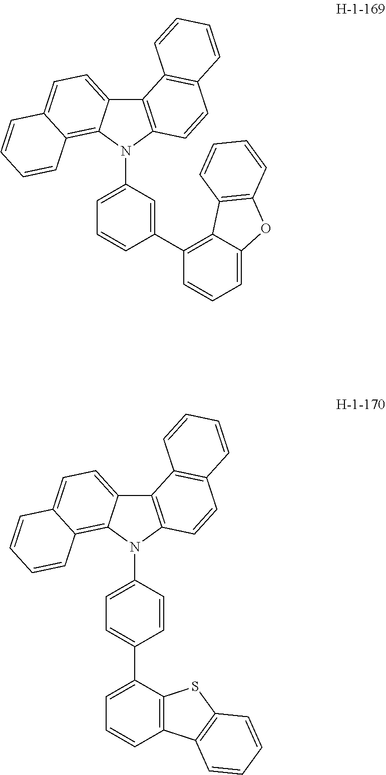 [SMILES: c1cc(-c2cccc3oc4ccccc4c23)cc(-n2c3ccc4ccccc4c3c3ccc4ccccc4c32)c1 ; c1ccc2c(c1)ccc1c2c2ccc3ccccc3c2n1-c1ccc(-c2cccc3c2sc2ccccc23)cc1]

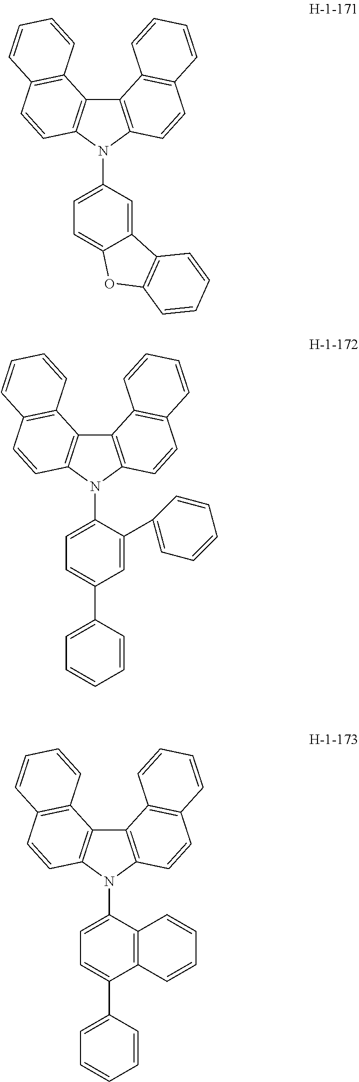

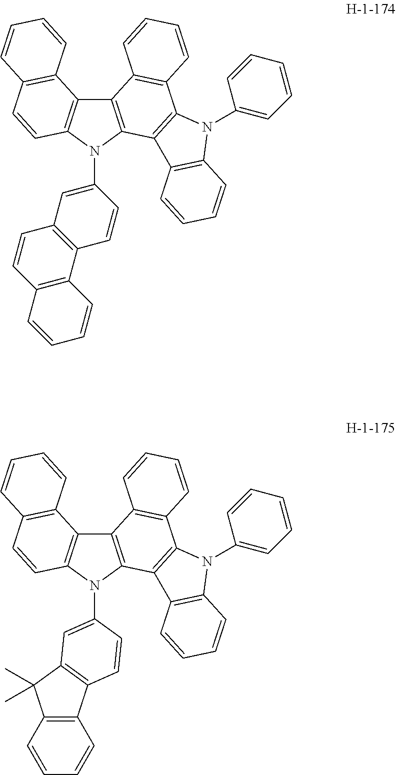 [SMILES: c1ccc(-n2c3ccccc3c3c2c2ccccc2c2c4c5ccccc5ccc4n(-c4ccc5c(ccc6ccccc65)c4)c23)cc1 ; CC1(C)c2ccccc2-c2ccc(-n3c4ccc5ccccc5c4c4c5ccccc5c5c(c6ccccc6n5-c5ccccc5)c43)cc21]

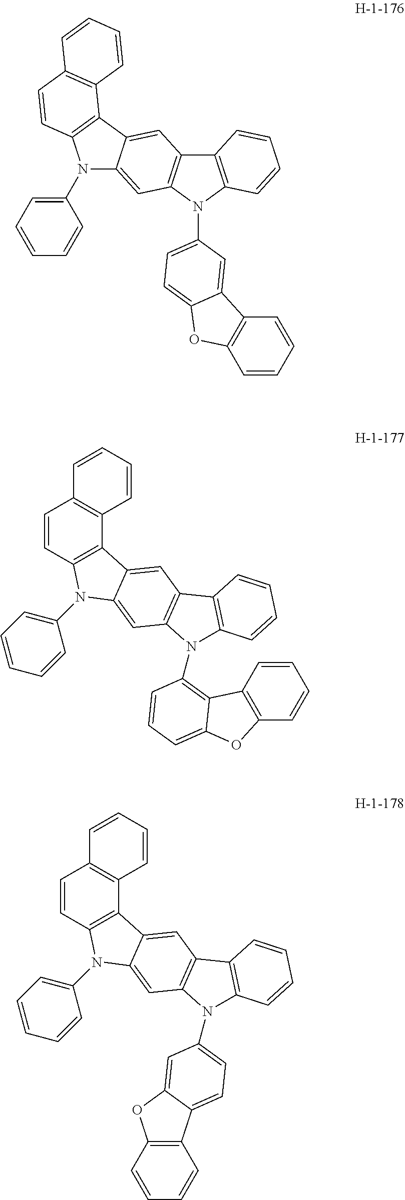 [SMILES: c1ccc(-n2c3cc4c(cc3c3c5ccccc5ccc32)c2ccccc2n4-c2ccc3oc4ccccc4c3c2)cc1 ; c1ccc(-n2c3cc4c(cc3c3c5ccccc5ccc32)c2ccccc2n4-c2cccc3oc4ccccc4c23)cc1 ; c1ccc(-n2c3cc4c(cc3c3c5ccccc5ccc32)c2ccccc2n4-c2ccc3c(c2)oc2ccccc23)cc1]

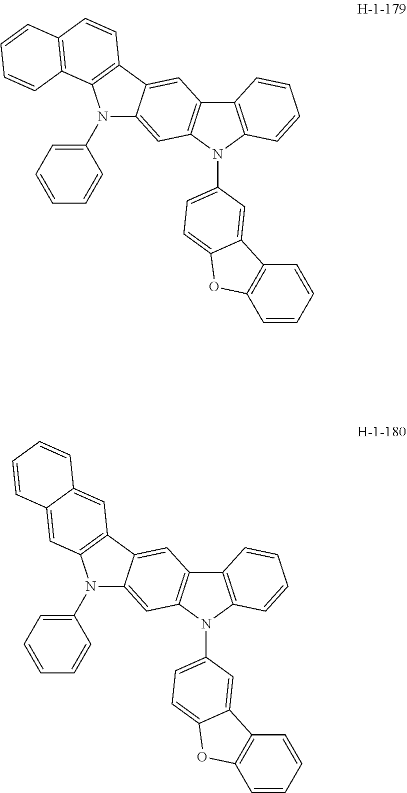 [SMILES: c1ccc(-n2c3cc4c(cc3c3ccc5ccccc5c32)c2ccccc2n4-c2ccc3oc4ccccc4c3c2)cc1 ; c1ccc(-n2c3cc4ccccc4cc3c3cc4c5ccccc5n(-c5ccc6oc7ccccc7c6c5)c4cc32)cc1]

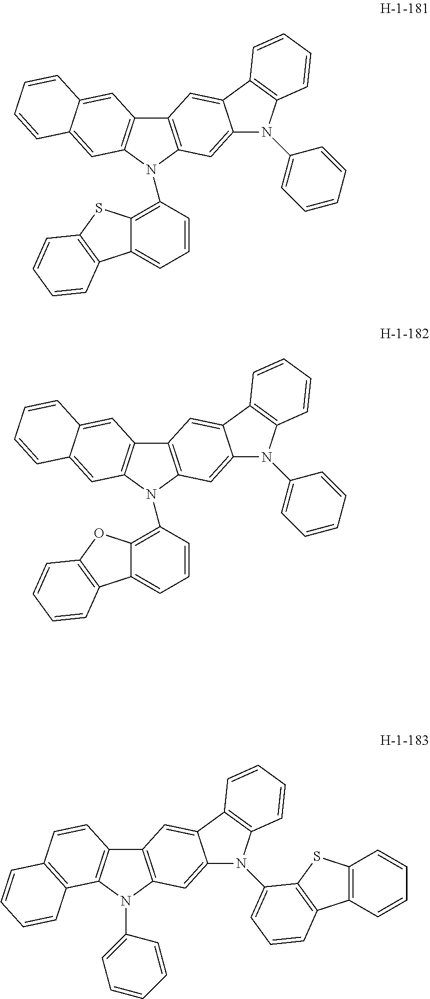 [SMILES: c1ccc(-n2c3ccccc3c3cc4c5cc6ccccc6cc5n(-c5cccc6c5sc5ccccc56)c4cc32)cc1 ; c1ccc(-n2c3ccccc3c3cc4c5cc6ccccc6cc5n(-c5cccc6c5oc5ccccc56)c4cc32)cc1 ; c1ccc(-n2c3cc4c(cc3c3ccc5ccccc5c32)c2ccccc2n4-c2cccc3c2sc2ccccc23)cc1]

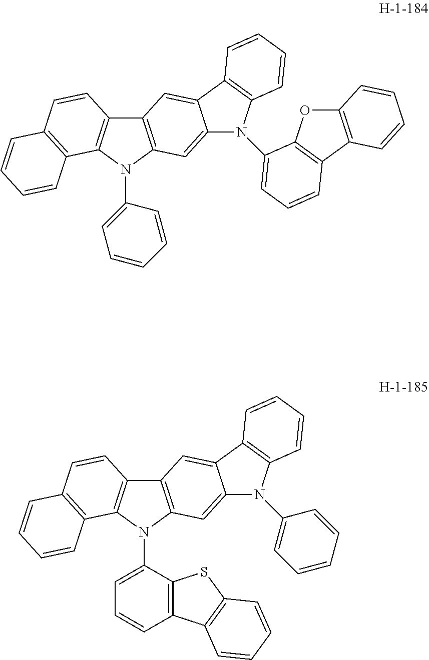 [SMILES: c1ccc(-n2c3cc4c(cc3c3ccc5ccccc5c32)c2ccccc2n4-c2cccc3c2oc2ccccc23)cc1 ; c1ccc(-n2c3ccccc3c3cc4c5ccc6ccccc6c5n(-c5cccc6c5sc5ccccc56)c4cc32)cc1]

Hereinafter, the compound represented by formula 2 will be described in more detail.

In formula 2, Y₁ represents O, S, CR₁₁R₁₂, or NR₁₃.

Herein, R₁₁ to R₁₃, each independently, represent hydrogen, deuterium, a halogen, a cyano, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, or a substituted or unsubstituted (3- to 30-membered)heteroaryl; or R₁₁ and R₁₂ may be linked to each other to form a ring. According to one embodiment of the present disclosure, R₁₁ and R₁₂, each independently, represent a substituted or unsubstituted (C1-C30)alkyl, or a substituted or unsubstituted (C6-C30)aryl; or may be linked to each other to form a substituted or unsubstituted, mono- or polycyclic, (3- to 30-membered) alicyclic or aromatic ring; and R₁₃ represents a substituted or unsubstituted (C6-C30)aryl, or a substituted or unsubstituted (3- to 30-membered)heteroaryl. According to another embodiment of the present disclosure, R₁₁ and R₁₂, each independently, represent a substituted or unsubstituted (C1-C10)alkyl, or a substituted or unsubstituted (C6-C18)aryl; or may be linked to each other to form a substituted or unsubstituted, polycyclic, (5- to 30-membered) aromatic ring; and R₁₃ represents a substituted or unsubstituted (C6-C25)aryl, or a substituted or unsubstituted (5- to 30-membered)heteroaryl. According to further embodiment of the present disclosure, R₁₁ and R₁₂, each independently, represent a substituted or unsubstituted (C1-C4)alkyl, or a substituted or unsubstituted (C6-C12)aryl; or may be linked to each other to form a substituted or unsubstituted, polycyclic, (5- to 25-membered) aromatic ring; and R₁₃ represents a substituted or unsubstituted (C6-C18)aryl, or a substituted or unsubstituted (5- to 25-membered)heteroaryl. For example, R₁₁ and R₁₂, each independently, represent a substituted or unsubstituted methyl, or a substituted or unsubstituted phenyl; or may be linked to each other to form a fluorene ring; and R₁₃ may represent a substituted or unsubstituted phenyl, a substituted or unsubstituted naphthyl, a substituted or unsubstituted m-biphenyl, a substituted or unsubstituted p-biphenyl, a substituted or unsubstituted fluorenyl, a substituted or unsubstituted phenanthrenyl, or a substituted or unsubstituted dibenzothiophenyl.

In formula 2, R₇ to R₉, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted (C1-C30)alkyl(C6-C30)arylamino, or a substituted or unsubstituted mono- or di-(C6-C30)arylamino; or may be linked to an adjacent substituent to form a ring(s). According to one embodiment of the present disclosure, R₇ and R₈, each independently, represent hydrogen, deuterium, a substituted or unsubstituted (C6-C30)aryl, or a substituted or unsubstituted (3- to 30-membered)heteroaryl; and R₉, each independently, represents hydrogen, deuterium, a substituted or unsubstituted (C6-C30)aryl, or a substituted or unsubstituted (3- to 30-membered)heteroaryl. According to another embodiment of the present disclosure, R₇ and R₈, each independently, represent hydrogen, a substituted or unsubstituted (C6-C25)aryl, or a substituted or unsubstituted (5- to 25-membered)heteroaryl; and R₉, each independently, represents hydrogen, a substituted or unsubstituted (C6-C25)aryl, or a substituted or unsubstituted (5- to 25-membered)heteroaryl containing at least one nitrogen. According to further embodiment of the present disclosure, R₇ and R₈, each independently, represent hydrogen, a substituted or unsubstituted (C6-C18)aryl, or a substituted or unsubstituted (5- to 18-membered)heteroaryl; and R₉, each independently, represents hydrogen, a substituted or unsubstituted (C6-C18)aryl, or a substituted or unsubstituted (5- to 18-membered)heteroaryl containing at least two nitrogens. Specifically, R₇ and R₈, each independently, may represent a substituted or unsubstituted phenyl, a substituted or unsubstituted naphthyl, a substituted or unsubstituted o-biphenyl, a substituted or unsubstituted m-biphenyl, a substituted or unsubstituted p-biphenyl, a substituted or unsubstituted carbazole, a substituted or unsubstituted dibenzofuranyl, a substituted or unsubstituted dibenzothiophenyl, a substituted or unsubstituted phenanthrenyl, etc.; and R₉, each independently, may represent hydrogen, a substituted or unsubstituted phenyl, a substituted or unsubstituted m-biphenyl, a substituted or unsubstituted naphthyl, a substituted or unsubstituted triazinyl, a substituted or unsubstituted quinoxalinyl, a substituted or unsubstituted benzoquinoxalinyl, a substituted or unsubstituted quinazolinyl, or a substituted or unsubstituted benzoquinazolinyl, etc.

In formula 2, at least one of R₁₃, R₈, and R₉ represents -L₂-(Ar₁)ₚ. According to one embodiment of the present disclosure, at least one of R₁₃ and R₉ represents -L₂-(Ar₁)ₚ. According to another embodiment of the present disclosure, R₉ represents -L₂-(Ar₁)ₚ.

In formula 2, L₂ represents a single bond, a substituted or unsubstituted (C6-C30)arylene, or a substituted or unsubstituted (3- to 30-membered)heteroarylene. According to one embodiment of the present disclosure, L₂ represents a single bond, or a substituted or unsubstituted (C6-C30)arylene, preferably a single bond, or a substituted or unsubstituted (C6-C25)arylene. According to another embodiment of the present disclosure, L₂ represents a single bond, or a substituted or unsubstituted (C6-C18)arylene. Specifically, L₂ may represent a single bond, a substituted or unsubstituted phenylene, a substituted or unsubstituted m-biphenylene, a substituted or unsubstituted naphthylene, a substituted or unsubstituted naphthylene-phenylene, a substituted or unsubstituted phenanthrenylene, etc.

In formula 2, Ar₁, each independently, represents a substituted or unsubstituted (3- to 30-membered)heteroaryl containing at least one nitrogen. According to one embodiment of the present disclosure, Ar₁, each independently, represents a substituted or unsubstituted (5- to 25-membered)heteroaryl containing at least one nitrogen. According to another embodiment of the present disclosure, Ar₁, each independently, represents a substituted or unsubstituted (5- to 18-membered)heteroaryl containing at least two nitrogens. Specifically, Ar₁, each independently, may represent a substituted or unsubstituted pyridyl, a substituted or unsubstituted pyrimidinyl, a substituted or unsubstituted triazinyl, a substituted or unsubstituted pyrazinyl, a substituted or unsubstituted pyridazinyl, a substituted or unsubstituted quinazolinyl, a substituted or unsubstituted benzoquinazolinyl, a substituted or unsubstituted quinoxalinyl, a substituted or unsubstituted benzoquinoxalinyl, a substituted or unsubstituted quinolyl, a substituted or unsubstituted benzoquinolyl, a substituted or unsubstituted isoquinolyl, a substituted or unsubstituted benzoisoquinolyl, a substituted or unsubstituted triazolyl, a substituted or unsubstituted pyrazolyl, a substituted or unsubstituted naphthyridinyl, or a substituted or unsubstituted benzothienopyrimidinyl; preferably a substituted or unsubstituted triazinyl, a substituted or unsubstituted quinoxalinyl, a substituted or unsubstituted benzoquinoxalinyl, a substituted or unsubstituted quinazolinyl, a substituted or unsubstituted benzoquinazolinyl, etc. For example, Ar₁, each independently, may represent a triazinyl, a quinazolinyl, a quinoxalinyl, a benzoquinazolinyl, or a benzoquinoxalinyl, unsubstituted or substituted with at least one of a substituted or unsubstituted (C6-C30)aryl(s) and a substituted or unsubstituted (5- to 30-membered)heteroaryl(s).

In formula 2, l and n, each independently, represent an integer of 1 to 4, and m and p, each independently, represent an integer of 1 or 2, where if l to n and p are an integer equal to 2 or more, each of R₇, each of R₈, each of R₉, and each of Ar₁ may be the same or different.

The compound represented by formula 2 may be represented by at least one of the following formulas 2-1 to 2-9:

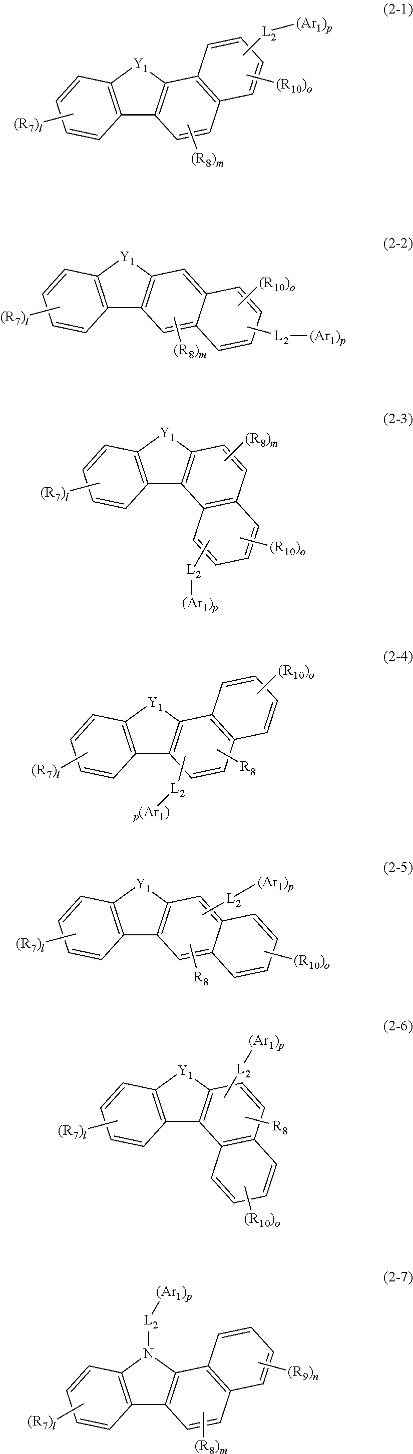

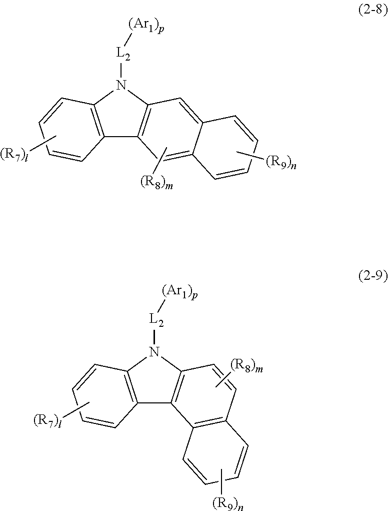

wherein

Y₁, L₂, Ar₁, R₇ to R₉, l to n, and p are as defined in formula 2;

R₁₀, each independently, has the same definition as R₉; and

o represents an integer of 1 to 3, where if o is an integer equal to 2 or more, each of R₁₀ may be the same or different.

According to one embodiment of the present disclosure, in formula 2-1, Y₁ represents O, S, CR₁₁R₁₂, or NR₁₃; R₁₁ and R₁₂, each independently, represent a substituted or unsubstituted (C1-C30)alkyl, or a substituted or unsubstituted (C6-C30)aryl; or may be linked to each other to form a substituted or unsubstituted, mono- or polycyclic, (3- to 30-membered) alicyclic or aromatic ring; R₁₃ represents a substituted or unsubstituted (C6-C30)aryl; R₇ and R₈, each independently, represent hydrogen, a substituted or unsubstituted (C6-C30)aryl, or a substituted or unsubstituted (3- to 30-membered)heteroaryl; L₂ represents a single bond, or a substituted or unsubstituted (C6-C30)arylene; Ar₁ represents a substituted or unsubstituted (3- to 30-membered)heteroaryl containing at least two nitrogens; and p represents 1.

According to one embodiment of the present disclosure, in formulas 2-2 and 2-3, Y₁ represents O, S, CR₁₁R₁₂, or NR₁₃; R₁₁ and R₁₂, each independently, represent a substituted or unsubstituted (C1-C30)alkyl, or a substituted or unsubstituted (C6-C30)aryl; or may be linked to each other to form a substituted or unsubstituted, mono- or polycyclic, (3- to 30-membered) alicyclic or aromatic ring; R₁₃ represents a substituted or unsubstituted (C6-C30)aryl, or a substituted or unsubstituted (3- to 30-membered)heteroaryl; both R₇ and R₈ represent hydrogen; L₂ represents a single bond, or a substituted or unsubstituted (C6-C30)arylene; Ar₁ represents a substituted or unsubstituted (3- to 30-membered)heteroaryl containing at least two nitrogens; and p represents 1.

According to one embodiment of the present disclosure, in formulas 2-4 to 2-6, Y₁ represents O, S, CR₁₁R₁₂, or NR₁₃; R₁₁ and R₁₂, each independently, represent a substituted or unsubstituted (C1-C30)alkyl; R₁₃ represents a substituted or unsubstituted (C6-C30)aryl; both R₇ and R₈ represent hydrogen; L₂ represents a single bond, or a substituted or unsubstituted (C6-C30)arylene; Ar₁ represents a substituted or unsubstituted (3- to 30-membered)heteroaryl containing at least two nitrogens; and p represents 1.

The compound represented by formula 2 may be at least one selected from the following compounds, but is not limited thereto.

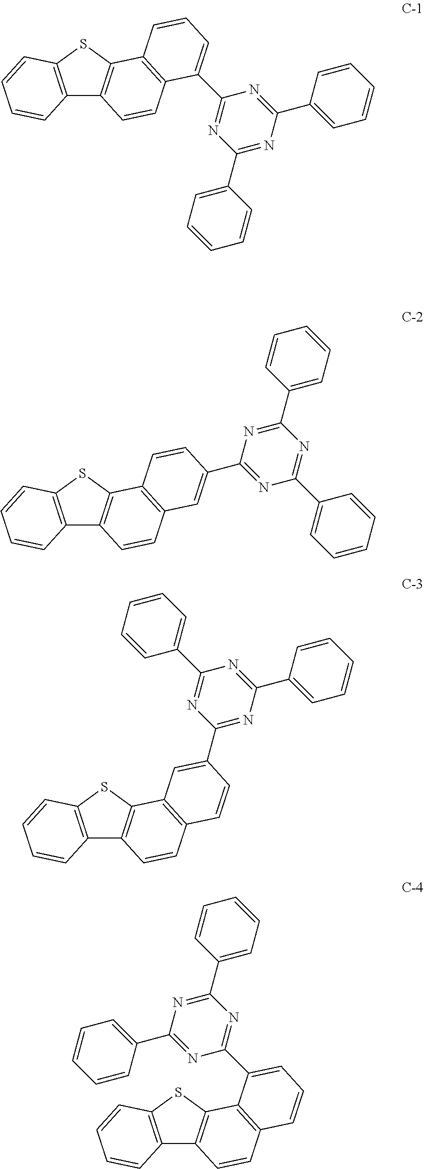 [SMILES: c1ccc(-c2nc(-c3ccccc3)nc(-c3cccc4c3ccc3c5ccccc5sc43)n2)cc1 ; c1ccc(-c2nc(-c3ccccc3)nc(-c3ccc4c(ccc5c6ccccc6sc45)c3)n2)cc1 ; c1ccc(-c2nc(-c3ccccc3)nc(-c3ccc4ccc5c6ccccc6sc5c4c3)n2)cc1 ; c1ccc(-c2nc(-c3ccccc3)nc(-c3cccc4ccc5c6ccccc6sc5c34)n2)cc1]

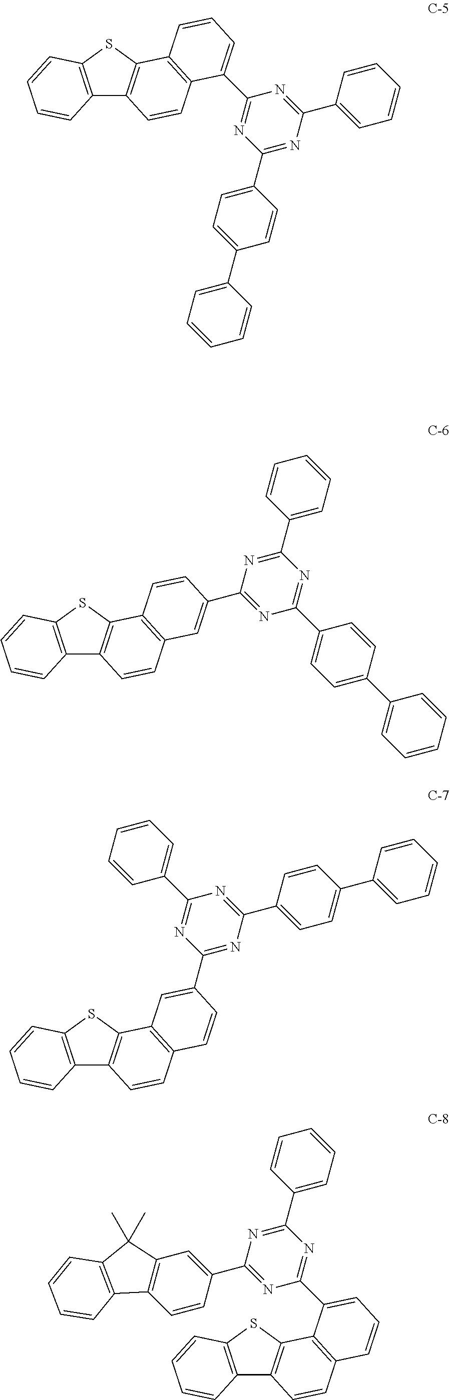 [SMILES: c1ccc(-c2ccc(-c3nc(-c4ccccc4)nc(-c4cccc5c4ccc4c6ccccc6sc54)n3)cc2)cc1 ; c1ccc(-c2ccc(-c3nc(-c4ccccc4)nc(-c4ccc5c(ccc6c7ccccc7sc56)c4)n3)cc2)cc1 ; c1ccc(-c2ccc(-c3nc(-c4ccccc4)nc(-c4ccc5ccc6c7ccccc7sc6c5c4)n3)cc2)cc1 ; CC1(C)c2ccccc2-c2ccc(-c3nc(-c4ccccc4)nc(-c4cccc5ccc6c7ccccc7sc6c45)n3)cc21]

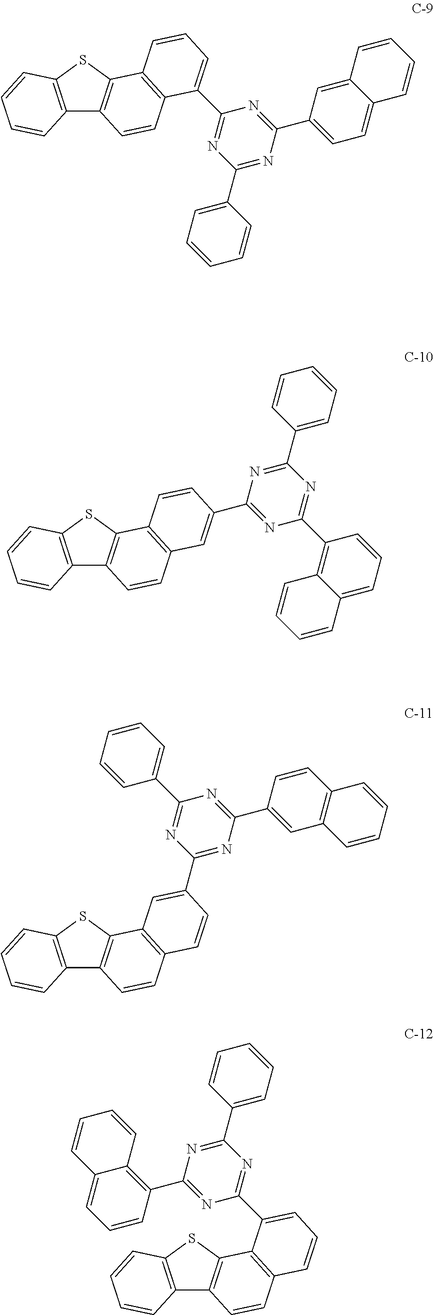 [SMILES: c1ccc(-c2nc(-c3ccc4ccccc4c3)nc(-c3cccc4c3ccc3c5ccccc5sc43)n2)cc1 ; c1ccc(-c2nc(-c3ccc4c(ccc5c6ccccc6sc45)c3)nc(-c3cccc4ccccc34)n2)cc1 ; c1ccc(-c2nc(-c3ccc4ccccc4c3)nc(-c3ccc4ccc5c6ccccc6sc5c4c3)n2)cc1 ; c1ccc(-c2nc(-c3cccc4ccccc34)nc(-c3cccc4ccc5c6ccccc6sc5c34)n2)cc1]

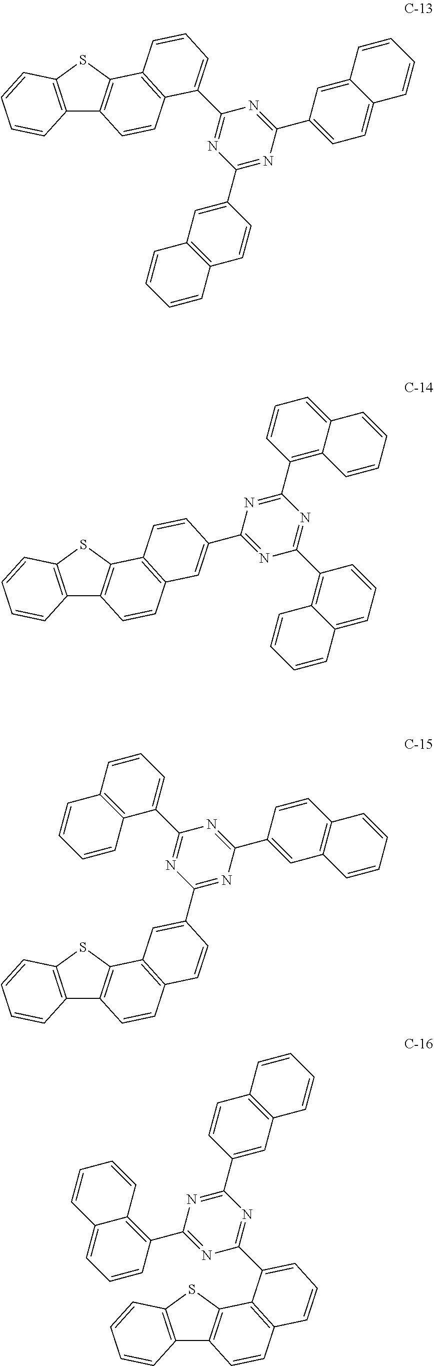 [SMILES: c1ccc2cc(-c3nc(-c4ccc5ccccc5c4)nc(-c4cccc5c4ccc4c6ccccc6sc54)n3)ccc2c1 ; c1ccc2c(-c3nc(-c4ccc5c(ccc6c7ccccc7sc56)c4)nc(-c4cccc5ccccc45)n3)cccc2c1 ; c1ccc2cc(-c3nc(-c4ccc5ccc6c7ccccc7sc6c5c4)nc(-c4cccc5ccccc45)n3)ccc2c1 ; c1ccc2cc(-c3nc(-c4cccc5ccccc45)nc(-c4cccc5ccc6c7ccccc7sc6c45)n3)ccc2c1]

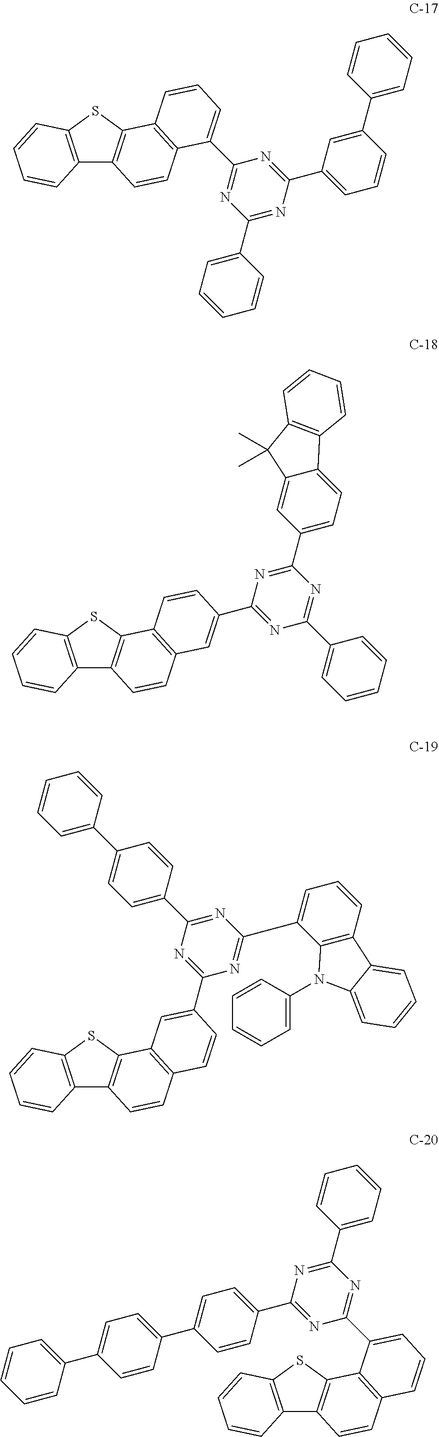 [SMILES: c1ccc(-c2cccc(-c3nc(-c4ccccc4)nc(-c4cccc5c4ccc4c6ccccc6sc54)n3)c2)cc1 ; CC1(C)c2ccccc2-c2ccc(-c3nc(-c4ccccc4)nc(-c4ccc5c(ccc6c7ccccc7sc56)c4)n3)cc21 ; c1ccc(-c2ccc(-c3nc(-c4ccc5ccc6c7ccccc7sc6c5c4)nc(-c4cccc5c6ccccc6n(-c6ccccc6)c45)n3)cc2)cc1 ; c1ccc(-c2ccc(-c3ccc(-c4nc(-c5ccccc5)nc(-c5cccc6ccc7c8ccccc8sc7c56)n4)cc3)cc2)cc1]

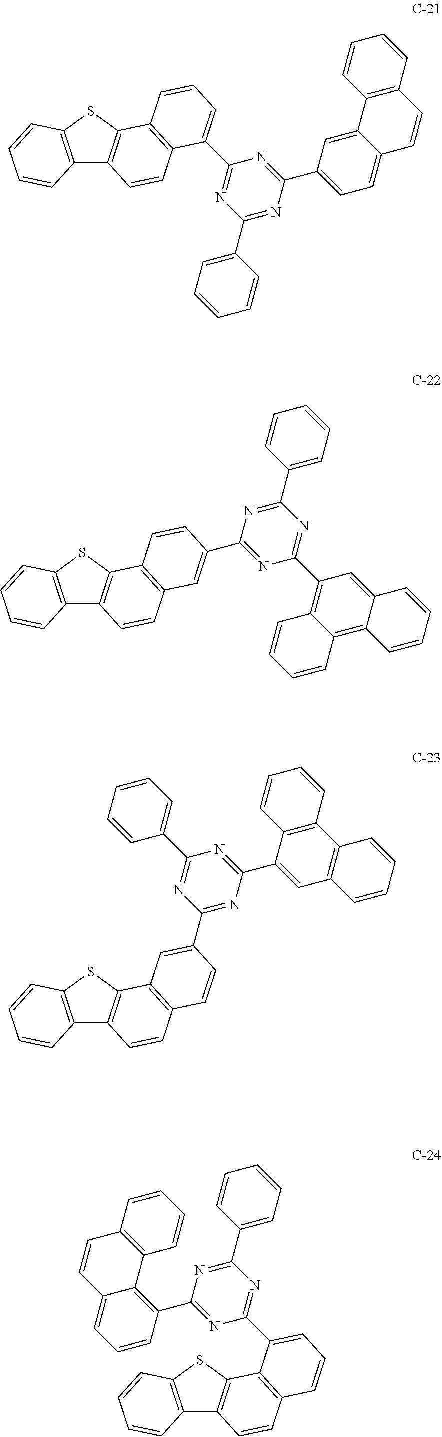 [SMILES: c1ccc(-c2nc(-c3ccc4ccc5ccccc5c4c3)nc(-c3cccc4c3ccc3c5ccccc5sc43)n2)cc1 ; c1ccc(-c2nc(-c3ccc4c(ccc5c6ccccc6sc45)c3)nc(-c3cc4ccccc4c4ccccc34)n2)cc1 ; c1ccc(-c2nc(-c3ccc4ccc5c6ccccc6sc5c4c3)nc(-c3cc4ccccc4c4ccccc34)n2)cc1 ; c1ccc(-c2nc(-c3cccc4ccc5ccccc5c34)nc(-c3cccc4ccc5c6ccccc6sc5c34)n2)cc1]

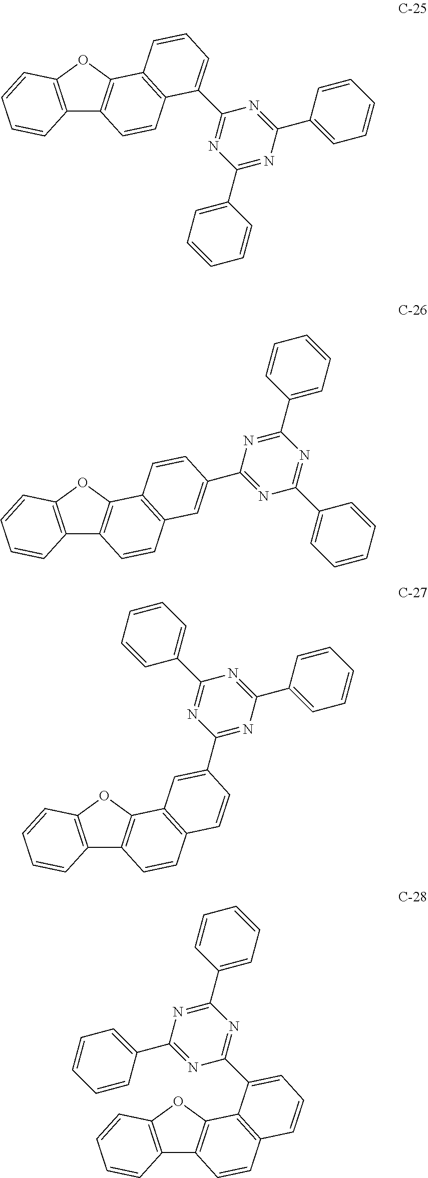 [SMILES: c1ccc(-c2nc(-c3ccccc3)nc(-c3cccc4c3ccc3c5ccccc5oc43)n2)cc1 ; c1ccc(-c2nc(-c3ccccc3)nc(-c3ccc4c(ccc5c6ccccc6oc45)c3)n2)cc1 ; c1ccc(-c2nc(-c3ccccc3)nc(-c3ccc4ccc5c6ccccc6oc5c4c3)n2)cc1 ; c1ccc(-c2nc(-c3ccccc3)nc(-c3cccc4ccc5c6ccccc6oc5c34)n2)cc1]

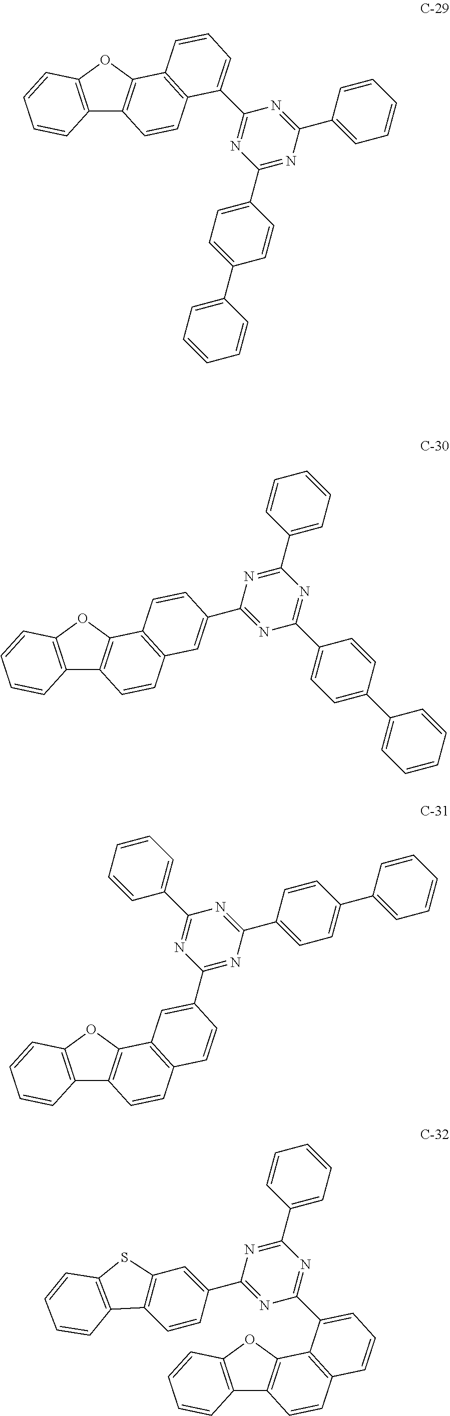 [SMILES: c1ccc(-c2ccc(-c3nc(-c4ccccc4)nc(-c4cccc5c4ccc4c6ccccc6oc54)n3)cc2)cc1 ; c1ccc(-c2ccc(-c3nc(-c4ccccc4)nc(-c4ccc5c(ccc6c7ccccc7oc56)c4)n3)cc2)cc1 ; c1ccc(-c2ccc(-c3nc(-c4ccccc4)nc(-c4ccc5ccc6c7ccccc7oc6c5c4)n3)cc2)cc1 ; c1ccc(-c2nc(-c3ccc4c(c3)sc3ccccc34)nc(-c3cccc4ccc5c6ccccc6oc5c34)n2)cc1]

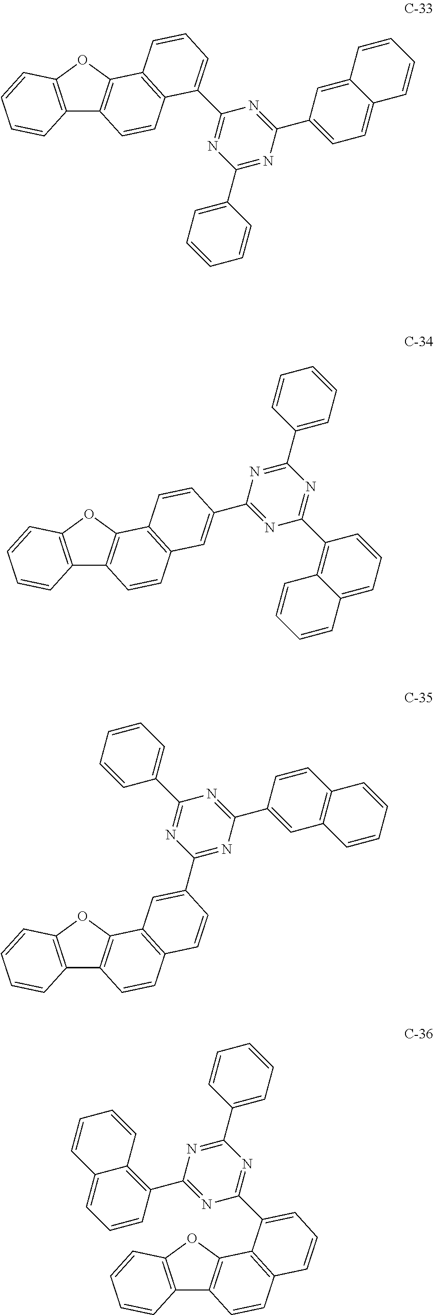 [SMILES: c1ccc(-c2nc(-c3ccc4ccccc4c3)nc(-c3cccc4c3ccc3c5ccccc5oc43)n2)cc1 ; c1ccc(-c2nc(-c3ccc4c(ccc5c6ccccc6oc45)c3)nc(-c3cccc4ccccc34)n2)cc1 ; c1ccc(-c2nc(-c3ccc4ccccc4c3)nc(-c3ccc4ccc5c6ccccc6oc5c4c3)n2)cc1 ; c1ccc(-c2nc(-c3cccc4ccccc34)nc(-c3cccc4ccc5c6ccccc6oc5c34)n2)cc1]

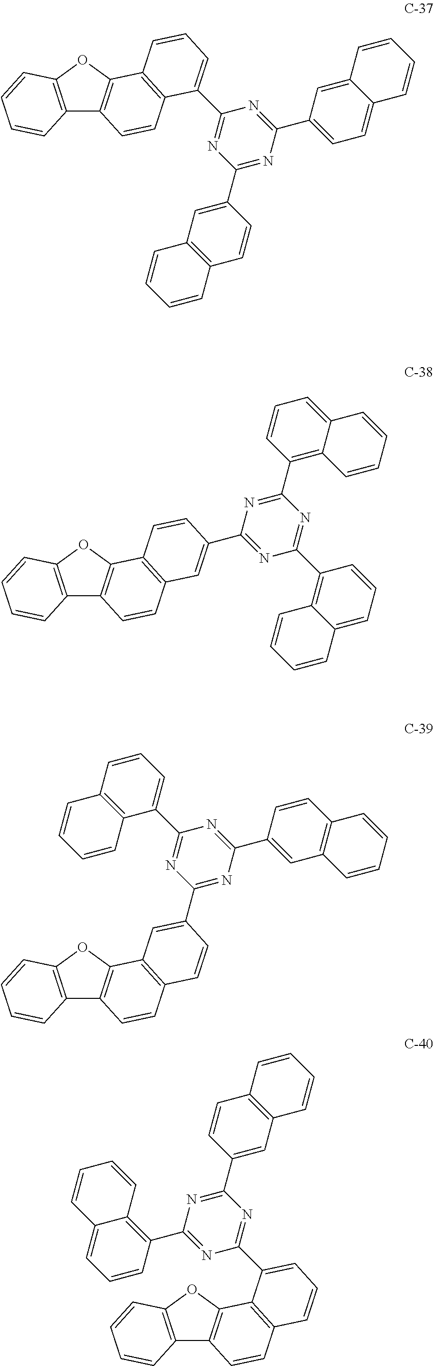 [SMILES: c1ccc2cc(-c3nc(-c4ccc5ccccc5c4)nc(-c4cccc5c4ccc4c6ccccc6oc54)n3)ccc2c1 ; c1ccc2c(-c3nc(-c4ccc5c(ccc6c7ccccc7oc56)c4)nc(-c4cccc5ccccc45)n3)cccc2c1 ; c1ccc2cc(-c3nc(-c4ccc5ccc6c7ccccc7oc6c5c4)nc(-c4cccc5ccccc45)n3)ccc2c1 ; c1ccc2cc(-c3nc(-c4cccc5ccccc45)nc(-c4cccc5ccc6c7ccccc7oc6c45)n3)ccc2c1]

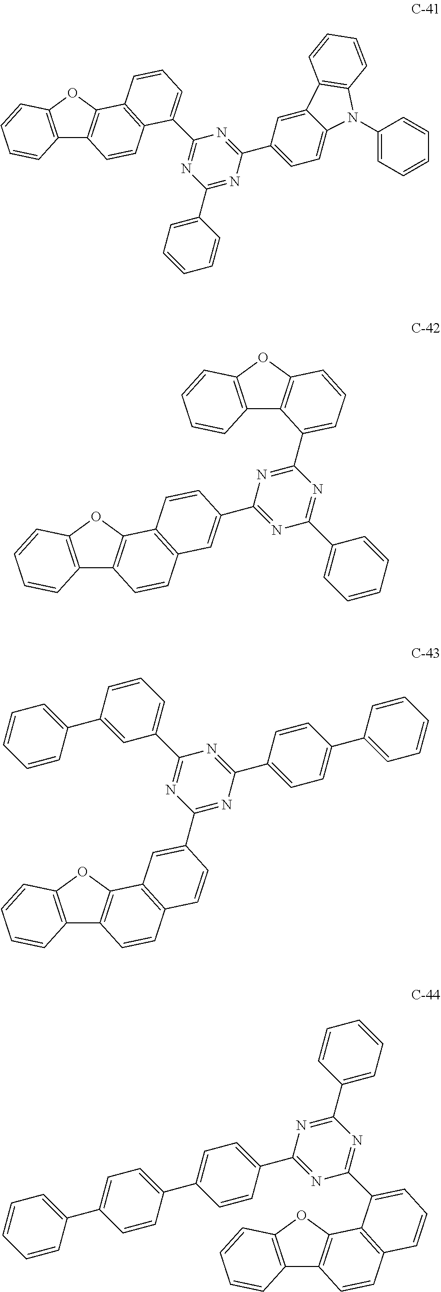 [SMILES: c1ccc(-c2nc(-c3ccc4c(c3)c3ccccc3n4-c3ccccc3)nc(-c3cccc4c3ccc3c5ccccc5oc43)n2)cc1 ; c1ccc(-c2nc(-c3ccc4c(ccc5c6ccccc6oc45)c3)nc(-c3cccc4oc5ccccc5c34)n2)cc1 ; c1ccc(-c2ccc(-c3nc(-c4cccc(-c5ccccc5)c4)nc(-c4ccc5ccc6c7ccccc7oc6c5c4)n3)cc2)cc1 ; c1ccc(-c2ccc(-c3ccc(-c4nc(-c5ccccc5)nc(-c5cccc6ccc7c8ccccc8oc7c56)n4)cc3)cc2)cc1]

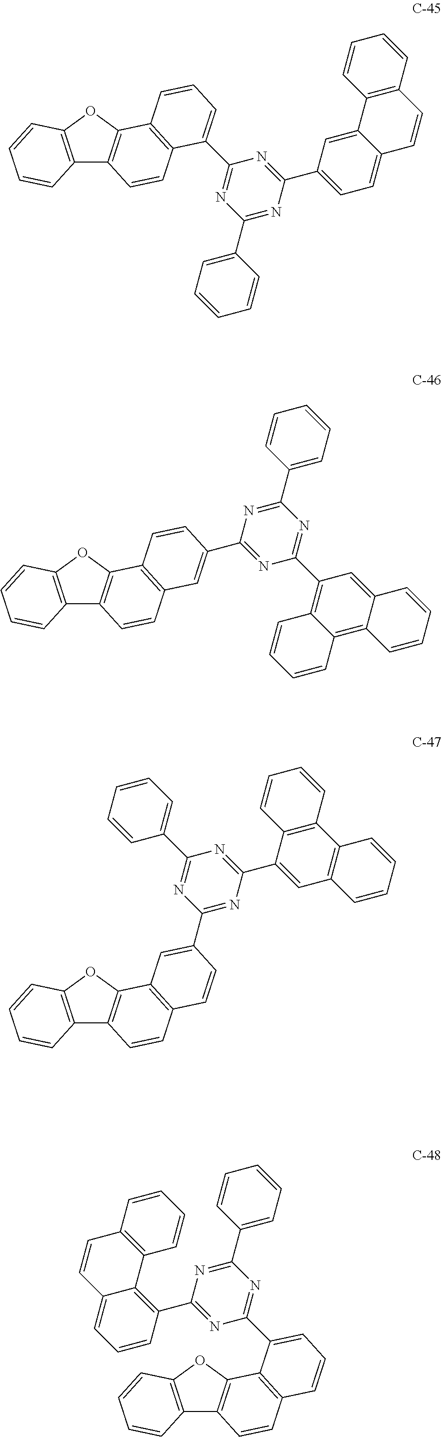 [SMILES: c1ccc(-c2nc(-c3ccc4ccc5ccccc5c4c3)nc(-c3cccc4c3ccc3c5ccccc5oc43)n2)cc1 ; c1ccc(-c2nc(-c3ccc4c(ccc5c6ccccc6oc45)c3)nc(-c3cc4ccccc4c4ccccc34)n2)cc1 ; c1ccc(-c2nc(-c3ccc4ccc5c6ccccc6oc5c4c3)nc(-c3cc4ccccc4c4ccccc34)n2)cc1 ; c1ccc(-c2nc(-c3cccc4ccc5ccccc5c34)nc(-c3cccc4ccc5c6ccccc6oc5c34)n2)cc1]

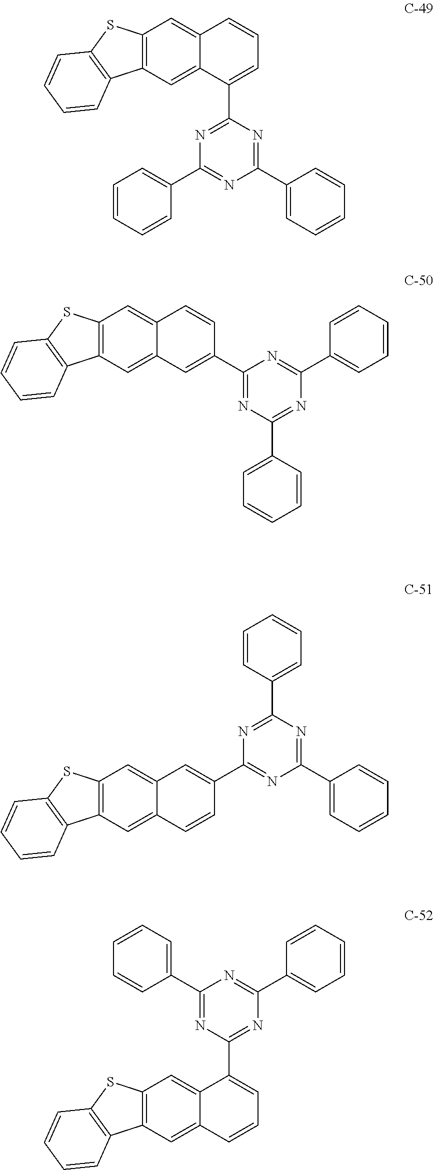 [SMILES: c1ccc(-c2nc(-c3ccccc3)nc(-c3cccc4cc5sc6ccccc6c5cc34)n2)cc1 ; c1ccc(-c2nc(-c3ccccc3)nc(-c3ccc4cc5sc6ccccc6c5cc4c3)n2)cc1 ; c1ccc(-c2nc(-c3ccccc3)nc(-c3ccc4cc5c(cc4c3)sc3ccccc35)n2)cc1 ; c1ccc(-c2nc(-c3ccccc3)nc(-c3cccc4cc5c(cc34)sc3ccccc35)n2)cc1]

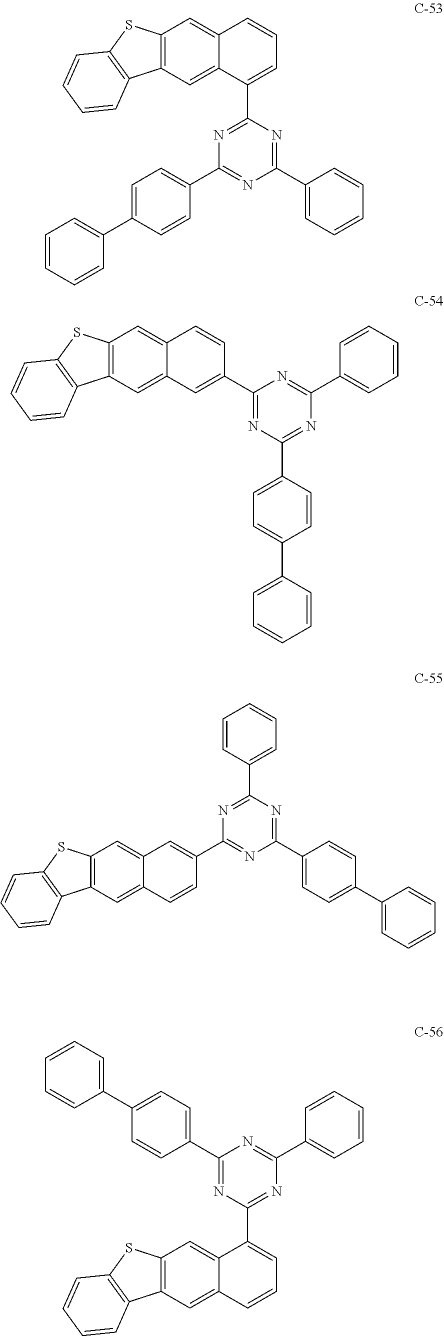 [SMILES: c1ccc(-c2ccc(-c3nc(-c4ccccc4)nc(-c4cccc5cc6sc7ccccc7c6cc45)n3)cc2)cc1 ; c1ccc(-c2ccc(-c3nc(-c4ccccc4)nc(-c4ccc5cc6sc7ccccc7c6cc5c4)n3)cc2)cc1 ; c1ccc(-c2ccc(-c3nc(-c4ccccc4)nc(-c4ccc5cc6c(cc5c4)sc4ccccc46)n3)cc2)cc1 ; c1ccc(-c2ccc(-c3nc(-c4ccccc4)nc(-c4cccc5cc6c(cc45)sc4ccccc46)n3)cc2)cc1]

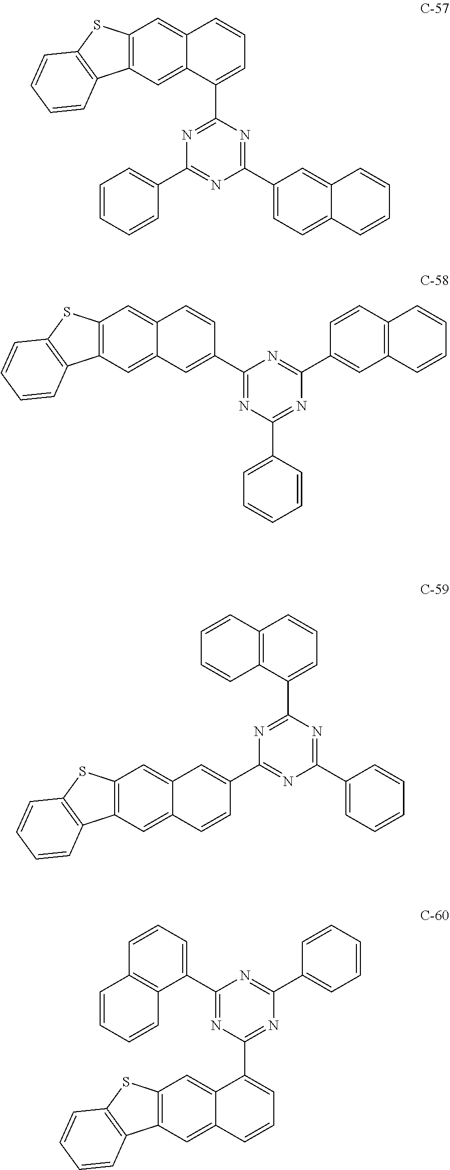 [SMILES: c1ccc(-c2nc(-c3ccc4ccccc4c3)nc(-c3cccc4cc5sc6ccccc6c5cc34)n2)cc1 ; c1ccc(-c2nc(-c3ccc4ccccc4c3)nc(-c3ccc4cc5sc6ccccc6c5cc4c3)n2)cc1 ; c1ccc(-c2nc(-c3ccc4cc5c(cc4c3)sc3ccccc35)nc(-c3cccc4ccccc34)n2)cc1 ; c1ccc(-c2nc(-c3cccc4ccccc34)nc(-c3cccc4cc5c(cc34)sc3ccccc35)n2)cc1]

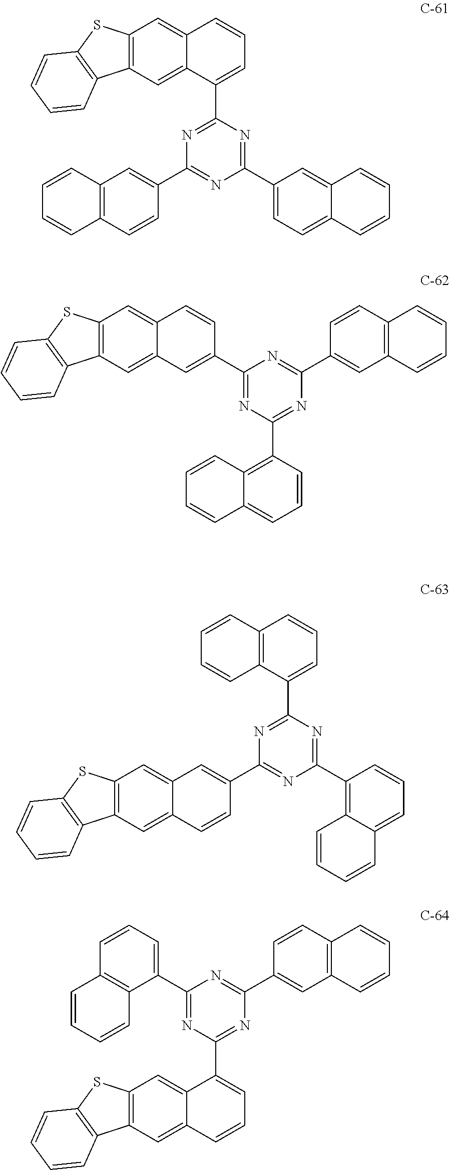

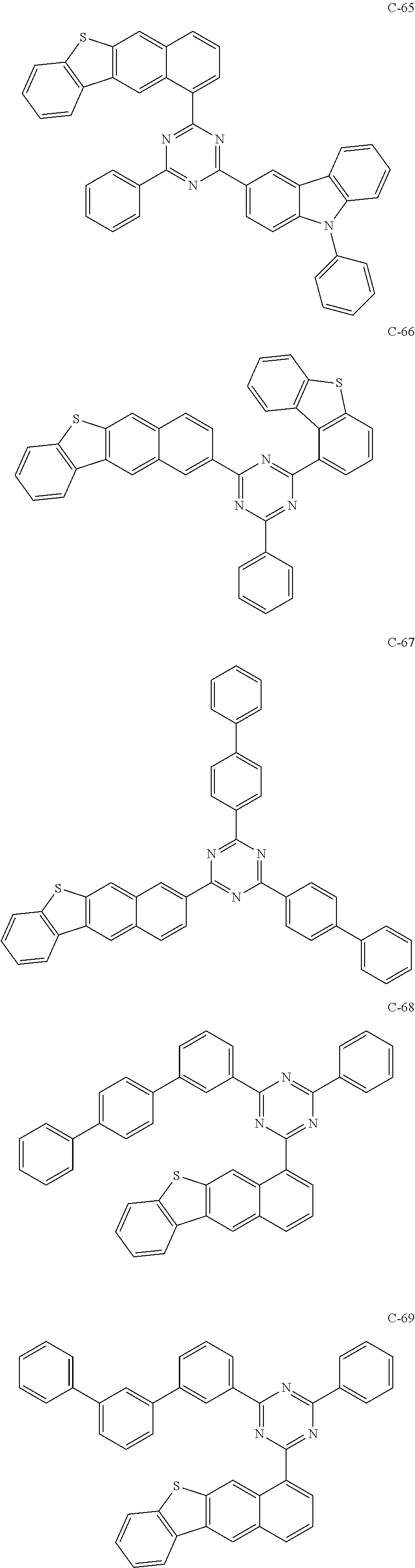 [SMILES: c1ccc(-c2nc(-c3ccc4c(c3)c3ccccc3n4-c3ccccc3)nc(-c3cccc4cc5sc6ccccc6c5cc34)n2)cc1 ; c1ccc(-c2nc(-c3ccc4cc5sc6ccccc6c5cc4c3)nc(-c3cccc4sc5ccccc5c34)n2)cc1 ; c1ccc(-c2ccc(-c3nc(-c4ccc(-c5ccccc5)cc4)nc(-c4ccc5cc6c(cc5c4)sc4ccccc46)n3)cc2)cc1 ; c1ccc(-c2ccc(-c3cccc(-c4nc(-c5ccccc5)nc(-c5cccc6cc7c(cc56)sc5ccccc57)n4)c3)cc2)cc1 ; c1ccc(-c2cccc(-c3cccc(-c4nc(-c5ccccc5)nc(-c5cccc6cc7c(cc56)sc5ccccc57)n4)c3)c2)cc1]

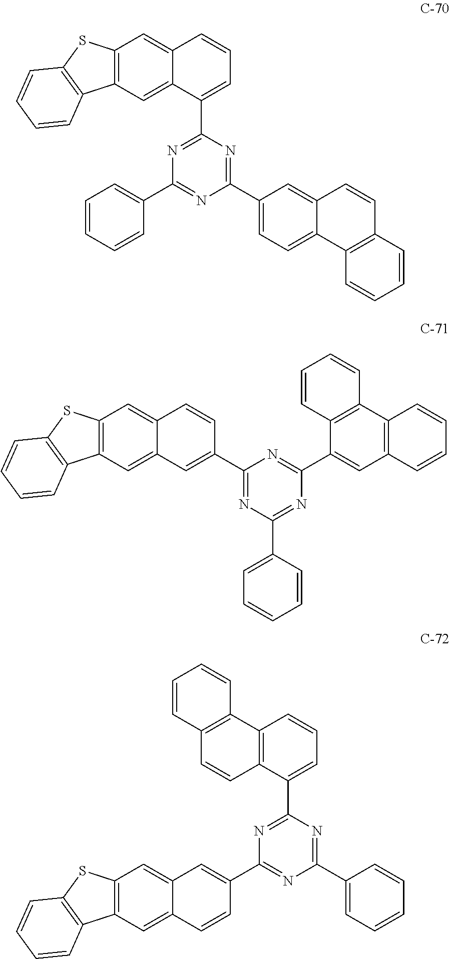 [SMILES: c1ccc(-c2nc(-c3ccc4c(ccc5ccccc54)c3)nc(-c3cccc4cc5sc6ccccc6c5cc34)n2)cc1 ; c1ccc(-c2nc(-c3ccc4cc5sc6ccccc6c5cc4c3)nc(-c3cc4ccccc4c4ccccc34)n2)cc1 ; c1ccc(-c2nc(-c3ccc4cc5c(cc4c3)sc3ccccc35)nc(-c3cccc4c3ccc3ccccc34)n2)cc1]

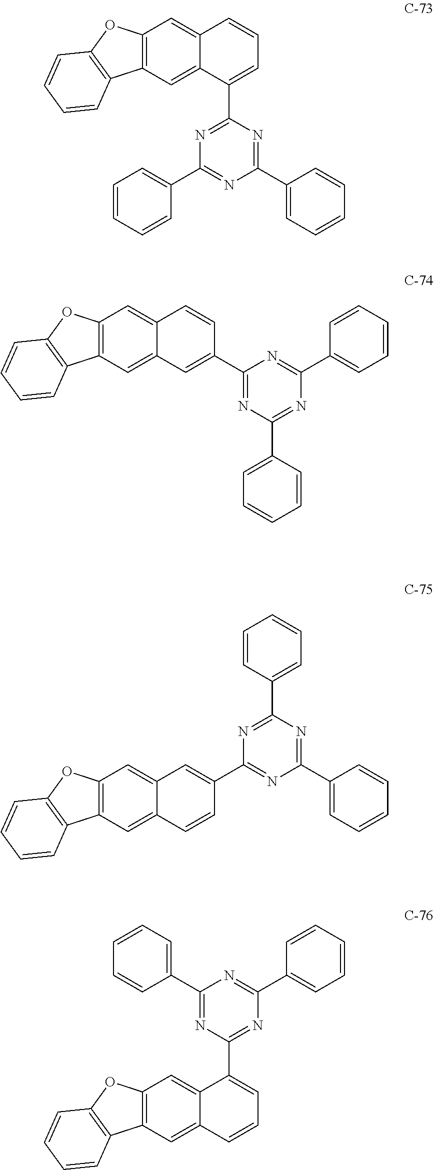 [SMILES: c1ccc(-c2nc(-c3ccccc3)nc(-c3cccc4cc5oc6ccccc6c5cc34)n2)cc1 ; c1ccc(-c2nc(-c3ccccc3)nc(-c3ccc4cc5oc6ccccc6c5cc4c3)n2)cc1 ; c1ccc(-c2nc(-c3ccccc3)nc(-c3ccc4cc5c(cc4c3)oc3ccccc35)n2)cc1 ; c1ccc(-c2nc(-c3ccccc3)nc(-c3cccc4cc5c(cc34)oc3ccccc35)n2)cc1]

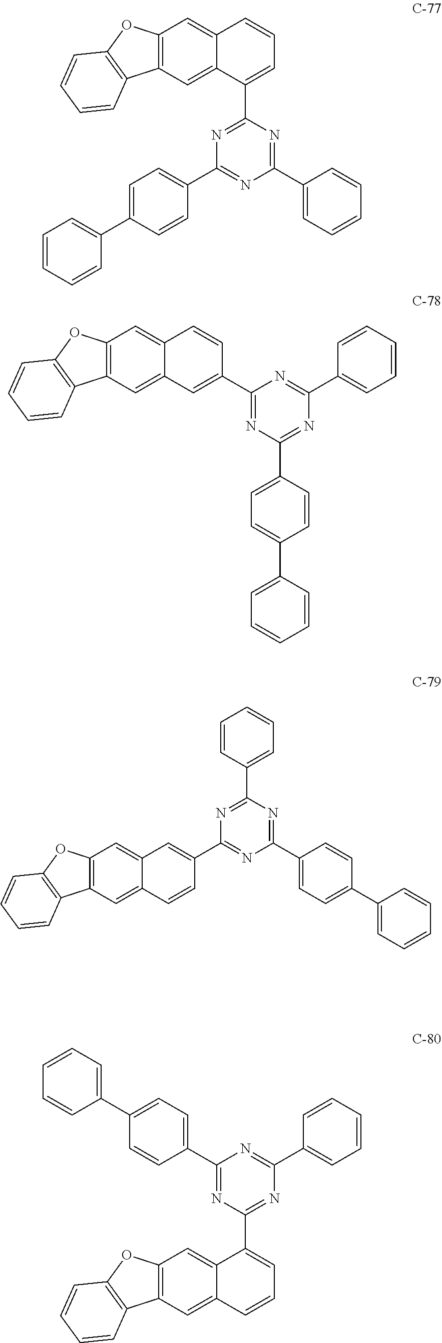 [SMILES: c1ccc(-c2ccc(-c3nc(-c4ccccc4)nc(-c4cccc5cc6oc7ccccc7c6cc45)n3)cc2)cc1 ; c1ccc(-c2ccc(-c3nc(-c4ccccc4)nc(-c4ccc5cc6oc7ccccc7c6cc5c4)n3)cc2)cc1 ; c1ccc(-c2ccc(-c3nc(-c4ccccc4)nc(-c4ccc5cc6c(cc5c4)oc4ccccc46)n3)cc2)cc1 ; c1ccc(-c2ccc(-c3nc(-c4ccccc4)nc(-c4cccc5cc6c(cc45)oc4ccccc46)n3)cc2)cc1]

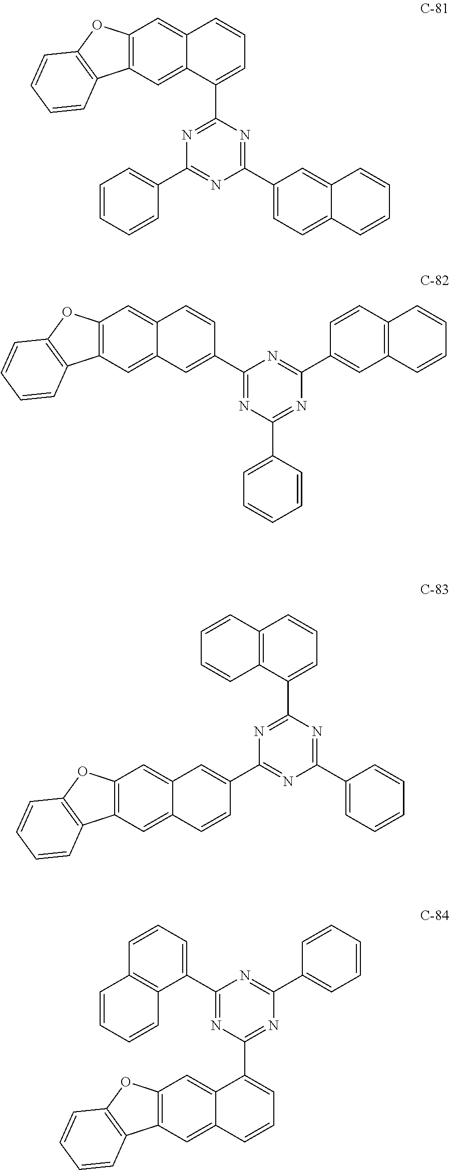 [SMILES: c1ccc(-c2nc(-c3ccc4ccccc4c3)nc(-c3cccc4cc5oc6ccccc6c5cc34)n2)cc1 ; c1ccc(-c2nc(-c3ccc4ccccc4c3)nc(-c3ccc4cc5oc6ccccc6c5cc4c3)n2)cc1 ; c1ccc(-c2nc(-c3ccc4cc5c(cc4c3)oc3ccccc35)nc(-c3cccc4ccccc34)n2)cc1 ; c1ccc(-c2nc(-c3cccc4ccccc34)nc(-c3cccc4cc5c(cc34)oc3ccccc35)n2)cc1]

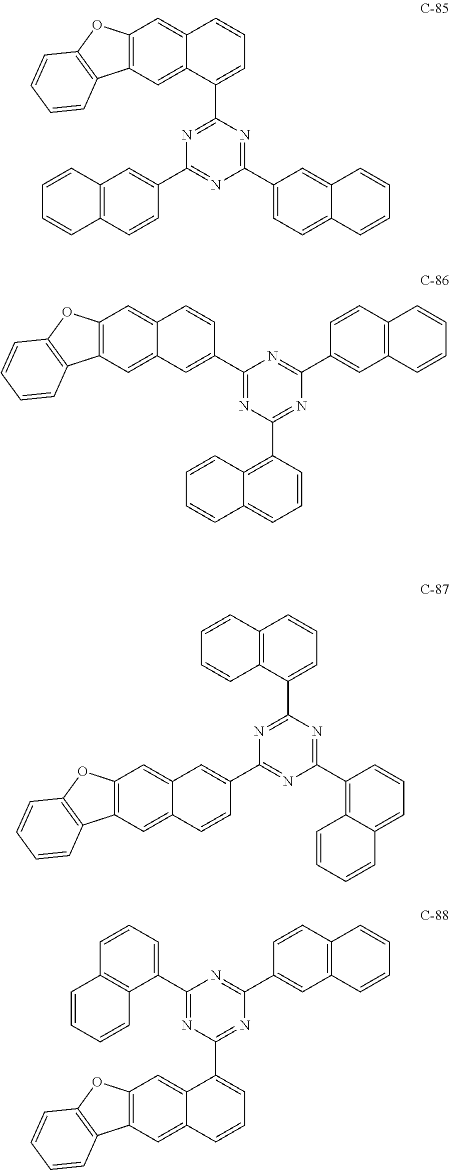

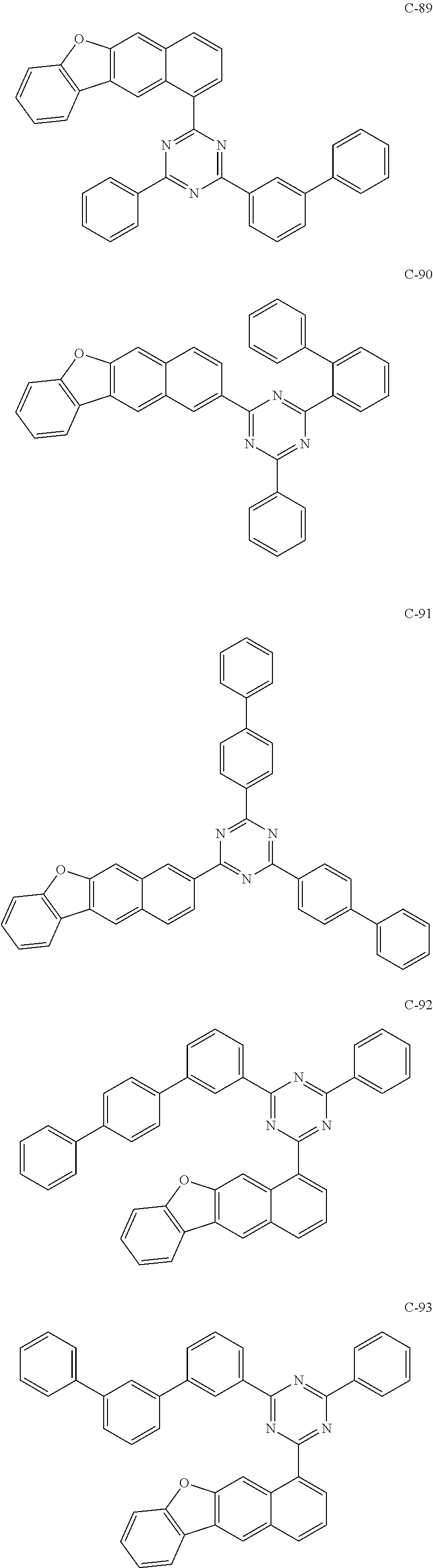 [SMILES: c1ccc(-c2cccc(-c3nc(-c4ccccc4)nc(-c4cccc5cc6oc7ccccc7c6cc45)n3)c2)cc1 ; c1ccc(-c2nc(-c3ccc4cc5oc6ccccc6c5cc4c3)nc(-c3ccccc3-c3ccccc3)n2)cc1 ; c1ccc(-c2ccc(-c3nc(-c4ccc(-c5ccccc5)cc4)nc(-c4ccc5cc6c(cc5c4)oc4ccccc46)n3)cc2)cc1 ; c1ccc(-c2ccc(-c3cccc(-c4nc(-c5ccccc5)nc(-c5cccc6cc7c(cc56)oc5ccccc57)n4)c3)cc2)cc1 ; c1ccc(-c2cccc(-c3cccc(-c4nc(-c5ccccc5)nc(-c5cccc6cc7c(cc56)oc5ccccc57)n4)c3)c2)cc1]

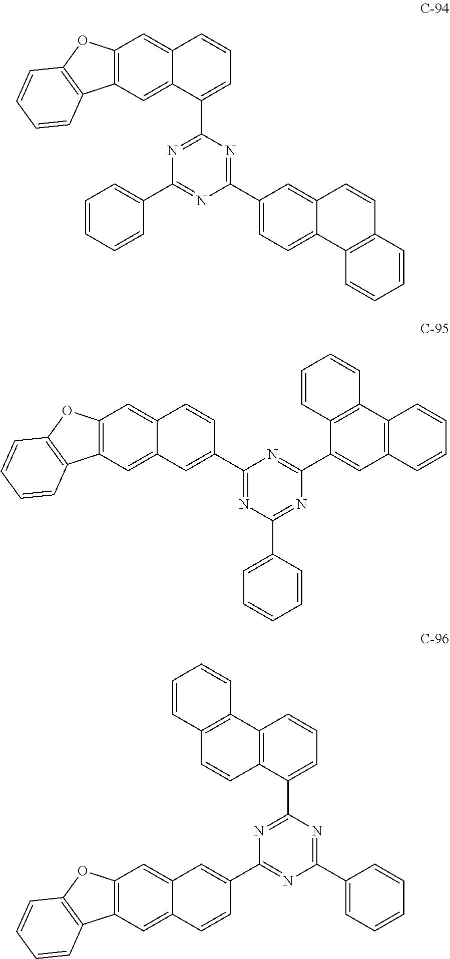 [SMILES: c1ccc(-c2nc(-c3ccc4c(ccc5ccccc54)c3)nc(-c3cccc4cc5oc6ccccc6c5cc34)n2)cc1 ; c1ccc(-c2nc(-c3ccc4cc5oc6ccccc6c5cc4c3)nc(-c3cc4ccccc4c4ccccc34)n2)cc1 ; c1ccc(-c2nc(-c3ccc4cc5c(cc4c3)oc3ccccc35)nc(-c3cccc4c3ccc3ccccc34)n2)cc1]

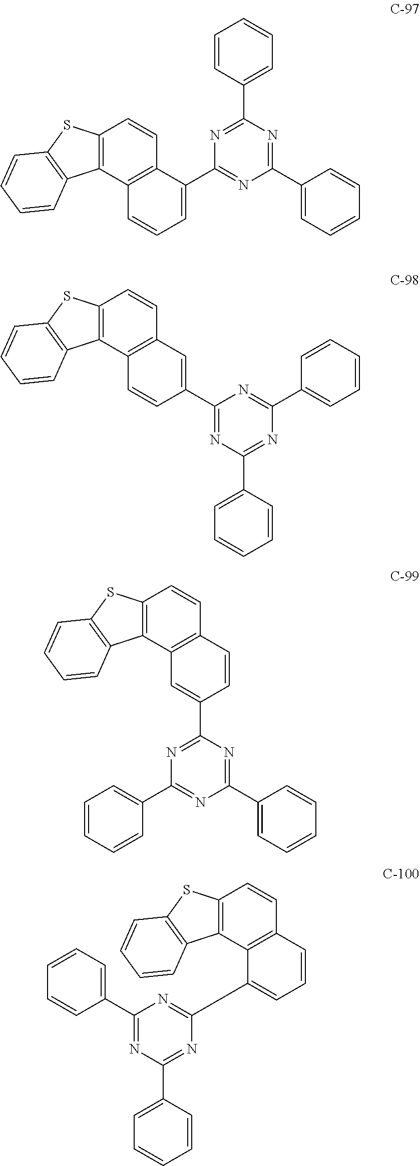 [SMILES: c1ccc(-c2nc(-c3ccccc3)nc(-c3cccc4c3ccc3sc5ccccc5c34)n2)cc1 ; c1ccc(-c2nc(-c3ccccc3)nc(-c3ccc4c(ccc5sc6ccccc6c54)c3)n2)cc1 ; c1ccc(-c2nc(-c3ccccc3)nc(-c3ccc4ccc5sc6ccccc6c5c4c3)n2)cc1 ; c1ccc(-c2nc(-c3ccccc3)nc(-c3cccc4ccc5sc6ccccc6c5c34)n2)cc1]

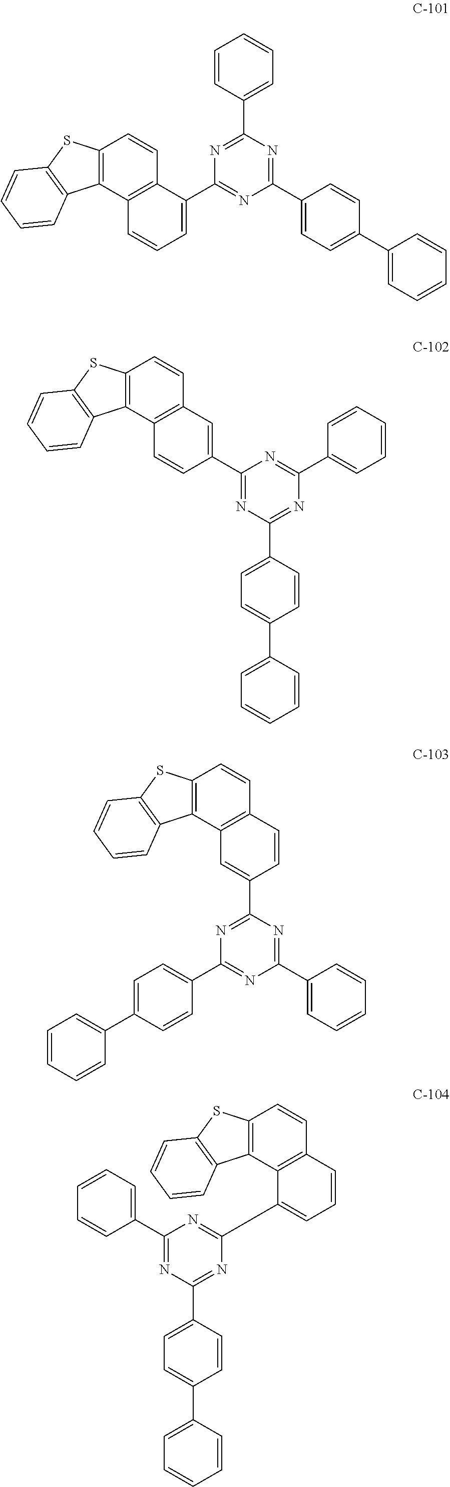 [SMILES: c1ccc(-c2ccc(-c3nc(-c4ccccc4)nc(-c4cccc5c4ccc4sc6ccccc6c45)n3)cc2)cc1 ; c1ccc(-c2ccc(-c3nc(-c4ccccc4)nc(-c4ccc5c(ccc6sc7ccccc7c65)c4)n3)cc2)cc1 ; c1ccc(-c2ccc(-c3nc(-c4ccccc4)nc(-c4ccc5ccc6sc7ccccc7c6c5c4)n3)cc2)cc1 ; c1ccc(-c2ccc(-c3nc(-c4ccccc4)nc(-c4cccc5ccc6sc7ccccc7c6c45)n3)cc2)cc1]

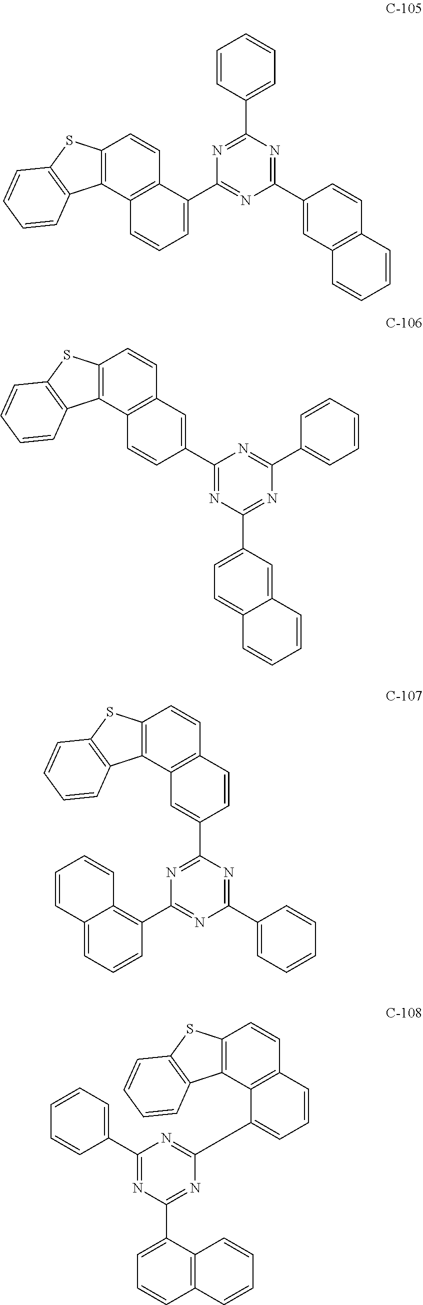 [SMILES: c1ccc(-c2nc(-c3ccc4ccccc4c3)nc(-c3cccc4c3ccc3sc5ccccc5c34)n2)cc1 ; c1ccc(-c2nc(-c3ccc4ccccc4c3)nc(-c3ccc4c(ccc5sc6ccccc6c54)c3)n2)cc1 ; c1ccc(-c2nc(-c3ccc4ccc5sc6ccccc6c5c4c3)nc(-c3cccc4ccccc34)n2)cc1 ; c1ccc(-c2nc(-c3cccc4ccccc34)nc(-c3cccc4ccc5sc6ccccc6c5c34)n2)cc1]

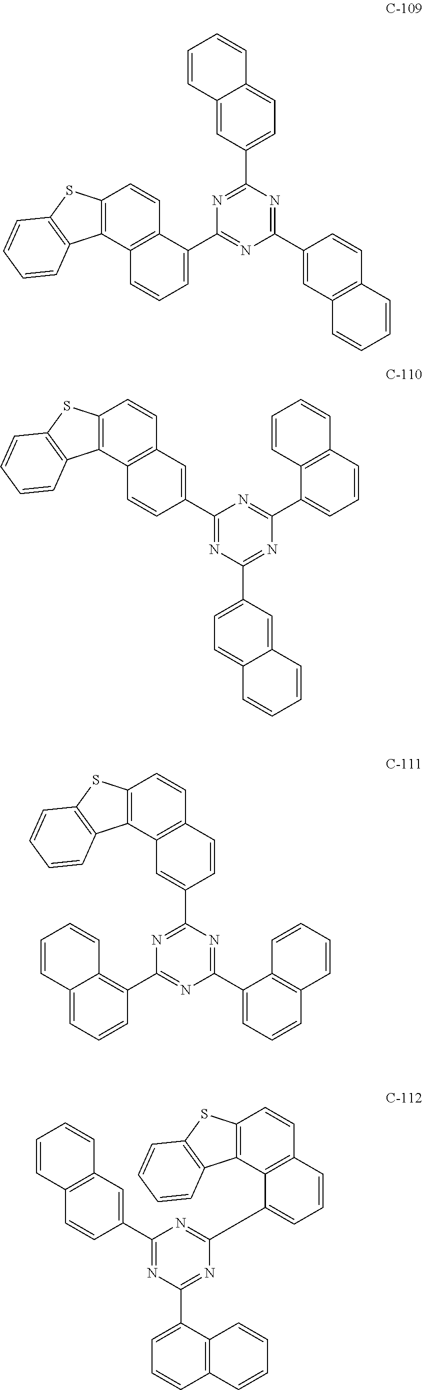 [SMILES: c1ccc2cc(-c3nc(-c4ccc5ccccc5c4)nc(-c4cccc5c4ccc4sc6ccccc6c45)n3)ccc2c1 ; c1ccc2cc(-c3nc(-c4ccc5c(ccc6sc7ccccc7c65)c4)nc(-c4cccc5ccccc45)n3)ccc2c1 ; c1ccc2c(-c3nc(-c4ccc5ccc6sc7ccccc7c6c5c4)nc(-c4cccc5ccccc45)n3)cccc2c1 ; c1ccc2cc(-c3nc(-c4cccc5ccccc45)nc(-c4cccc5ccc6sc7ccccc7c6c45)n3)ccc2c1]

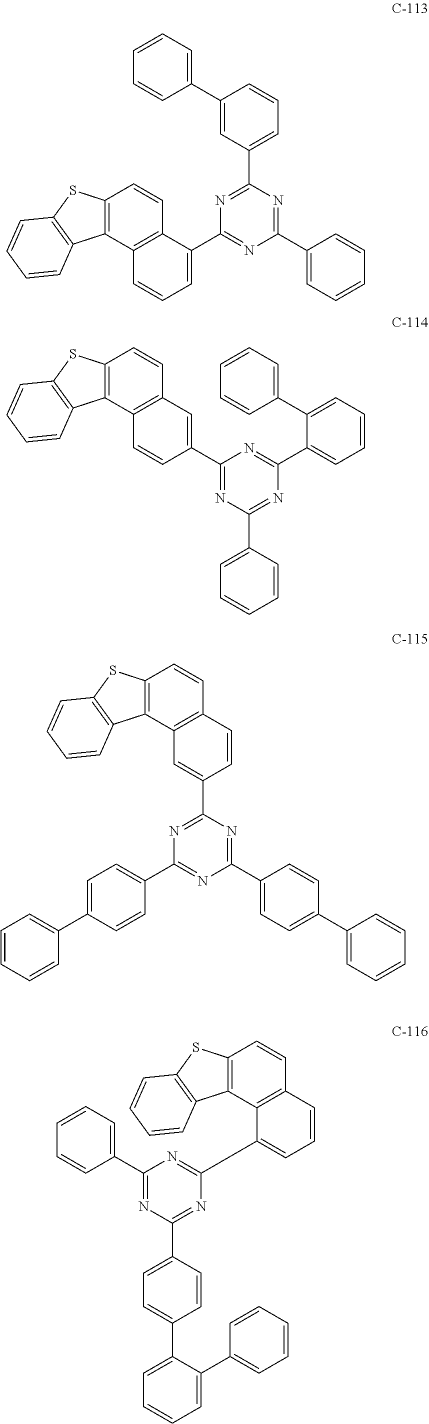 [SMILES: c1ccc(-c2cccc(-c3nc(-c4ccccc4)nc(-c4cccc5c4ccc4sc6ccccc6c45)n3)c2)cc1 ; c1ccc(-c2nc(-c3ccc4c(ccc5sc6ccccc6c54)c3)nc(-c3ccccc3-c3ccccc3)n2)cc1 ; c1ccc(-c2ccc(-c3nc(-c4ccc(-c5ccccc5)cc4)nc(-c4ccc5ccc6sc7ccccc7c6c5c4)n3)cc2)cc1 ; c1ccc(-c2nc(-c3ccc(-c4ccccc4-c4ccccc4)cc3)nc(-c3cccc4ccc5sc6ccccc6c5c34)n2)cc1]

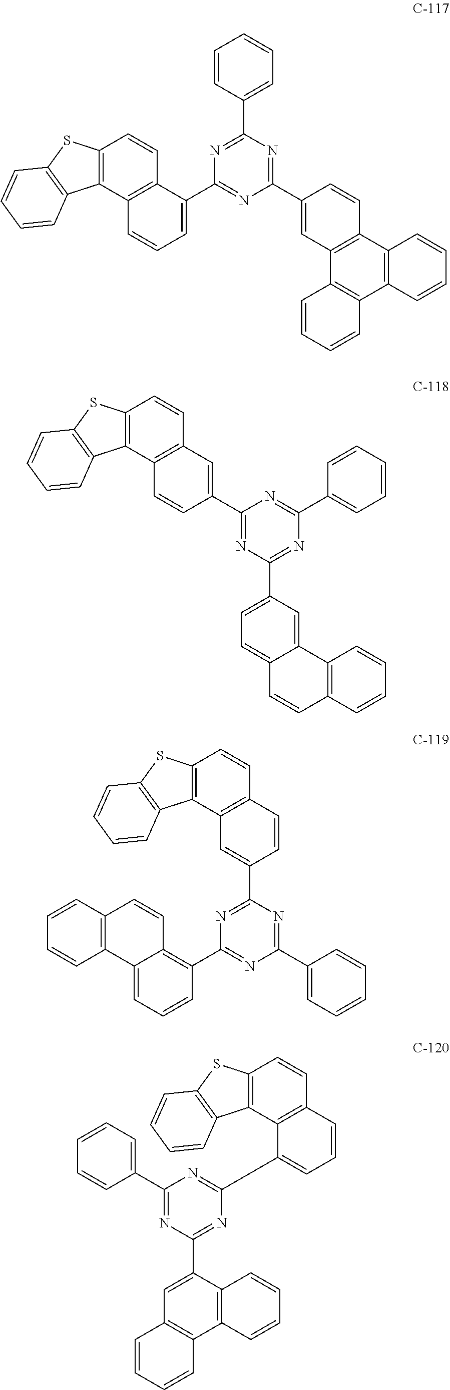 [SMILES: c1ccc(-c2nc(-c3ccc4c5ccccc5c5ccccc5c4c3)nc(-c3cccc4c3ccc3sc5ccccc5c34)n2)cc1 ; c1ccc(-c2nc(-c3ccc4c(ccc5sc6ccccc6c54)c3)nc(-c3ccc4ccc5ccccc5c4c3)n2)cc1 ; c1ccc(-c2nc(-c3ccc4ccc5sc6ccccc6c5c4c3)nc(-c3cccc4c3ccc3ccccc34)n2)cc1 ; c1ccc(-c2nc(-c3cc4ccccc4c4ccccc34)nc(-c3cccc4ccc5sc6ccccc6c5c34)n2)cc1]

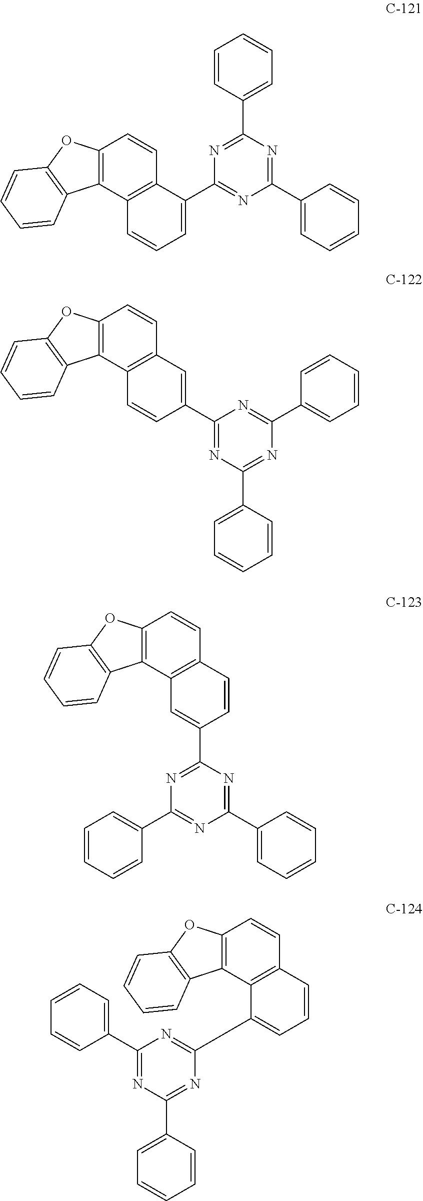 [SMILES: c1ccc(-c2nc(-c3ccccc3)nc(-c3cccc4c3ccc3oc5ccccc5c34)n2)cc1 ; c1ccc(-c2nc(-c3ccccc3)nc(-c3ccc4c(ccc5oc6ccccc6c54)c3)n2)cc1 ; c1ccc(-c2nc(-c3ccccc3)nc(-c3ccc4ccc5oc6ccccc6c5c4c3)n2)cc1 ; c1ccc(-c2nc(-c3ccccc3)nc(-c3cccc4ccc5oc6ccccc6c5c34)n2)cc1]

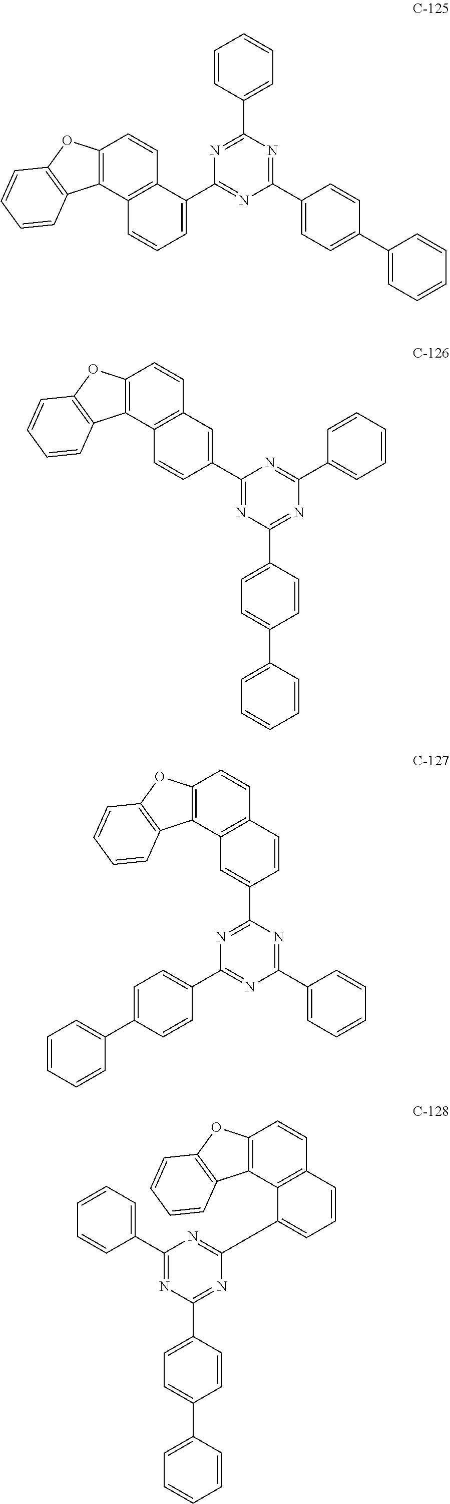 [SMILES: c1ccc(-c2ccc(-c3nc(-c4ccccc4)nc(-c4cccc5c4ccc4oc6ccccc6c45)n3)cc2)cc1 ; c1ccc(-c2ccc(-c3nc(-c4ccccc4)nc(-c4ccc5c(ccc6oc7ccccc7c65)c4)n3)cc2)cc1 ; c1ccc(-c2ccc(-c3nc(-c4ccccc4)nc(-c4ccc5ccc6oc7ccccc7c6c5c4)n3)cc2)cc1 ; c1ccc(-c2ccc(-c3nc(-c4ccccc4)nc(-c4cccc5ccc6oc7ccccc7c6c45)n3)cc2)cc1]

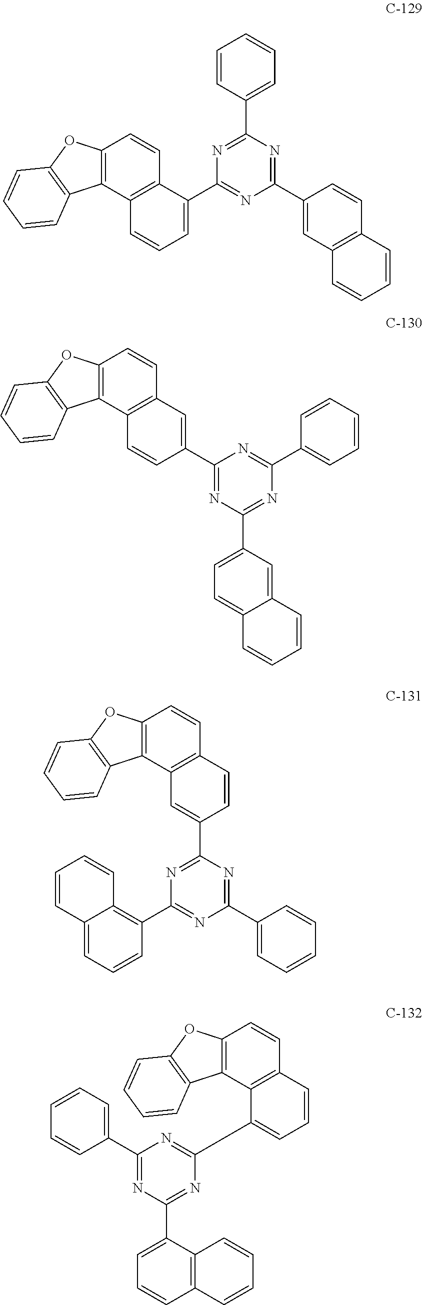 [SMILES: c1ccc(-c2nc(-c3ccc4ccccc4c3)nc(-c3cccc4c3ccc3oc5ccccc5c34)n2)cc1 ; c1ccc(-c2nc(-c3ccc4ccccc4c3)nc(-c3ccc4c(ccc5oc6ccccc6c54)c3)n2)cc1 ; c1ccc(-c2nc(-c3ccc4ccc5oc6ccccc6c5c4c3)nc(-c3cccc4ccccc34)n2)cc1 ; c1ccc(-c2nc(-c3cccc4ccccc34)nc(-c3cccc4ccc5oc6ccccc6c5c34)n2)cc1]

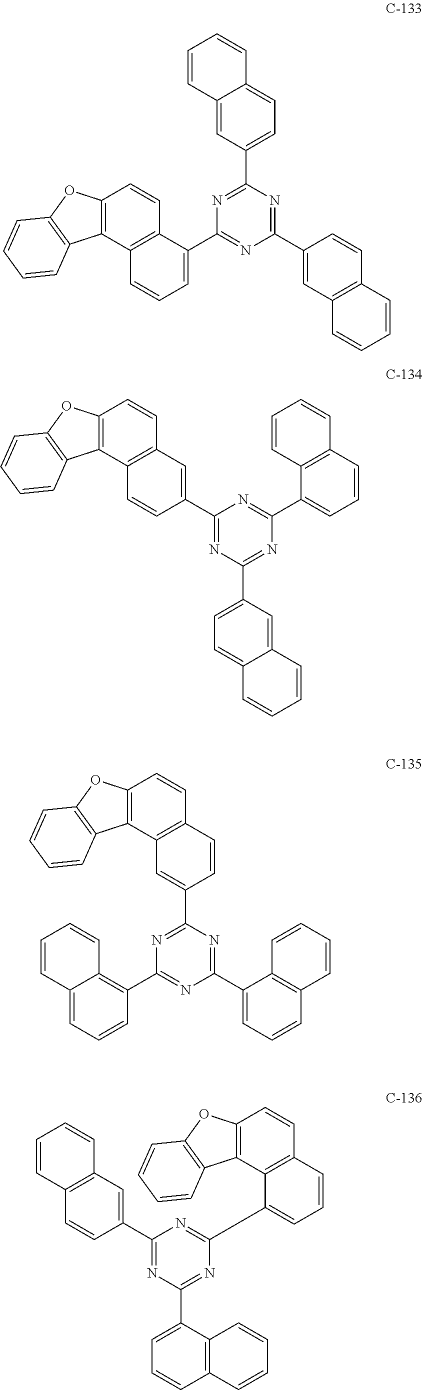 [SMILES: c1ccc2cc(-c3nc(-c4ccc5ccccc5c4)nc(-c4cccc5c4ccc4oc6ccccc6c45)n3)ccc2c1 ; c1ccc2cc(-c3nc(-c4ccc5c(ccc6oc7ccccc7c65)c4)nc(-c4cccc5ccccc45)n3)ccc2c1 ; c1ccc2c(-c3nc(-c4ccc5ccc6oc7ccccc7c6c5c4)nc(-c4cccc5ccccc45)n3)cccc2c1 ; c1ccc2cc(-c3nc(-c4cccc5ccccc45)nc(-c4cccc5ccc6oc7ccccc7c6c45)n3)ccc2c1]

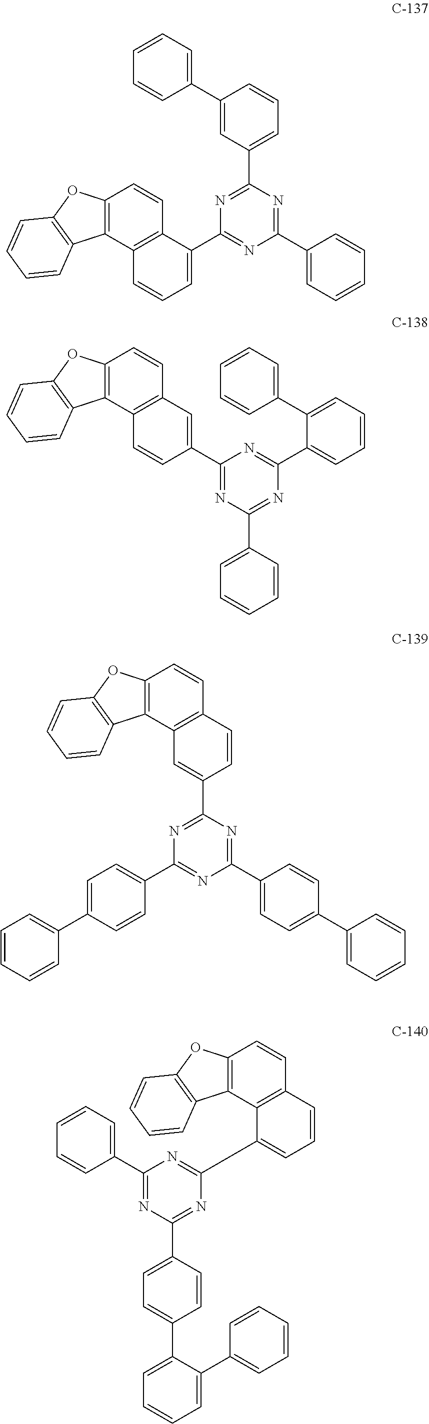 [SMILES: c1ccc(-c2cccc(-c3nc(-c4ccccc4)nc(-c4cccc5c4ccc4oc6ccccc6c45)n3)c2)cc1 ; c1ccc(-c2nc(-c3ccc4c(ccc5oc6ccccc6c54)c3)nc(-c3ccccc3-c3ccccc3)n2)cc1 ; c1ccc(-c2ccc(-c3nc(-c4ccc(-c5ccccc5)cc4)nc(-c4ccc5ccc6oc7ccccc7c6c5c4)n3)cc2)cc1 ; c1ccc(-c2nc(-c3ccc(-c4ccccc4-c4ccccc4)cc3)nc(-c3cccc4ccc5oc6ccccc6c5c34)n2)cc1]

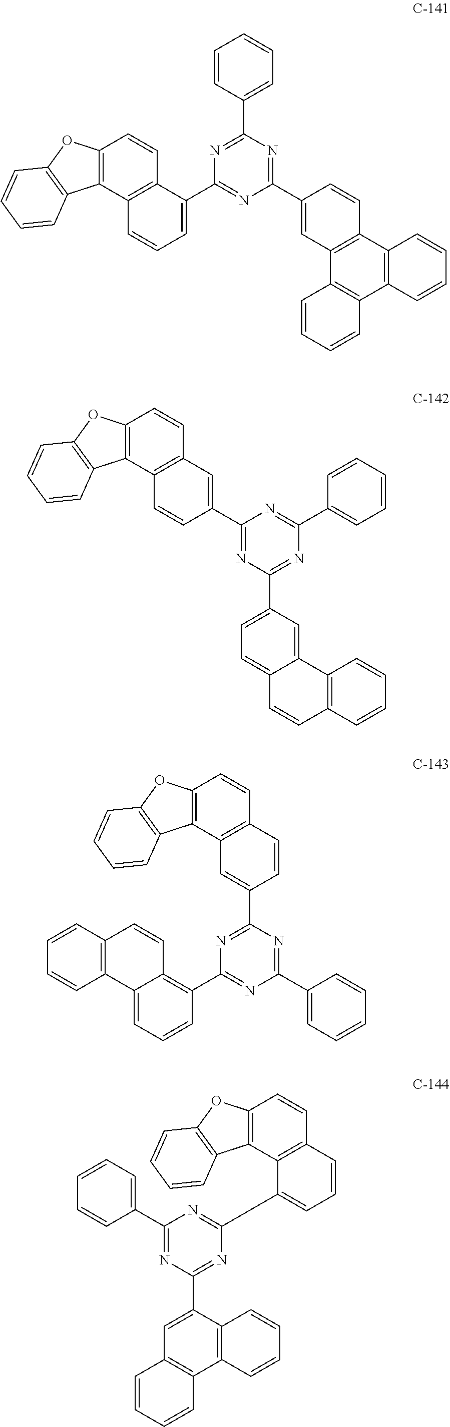 [SMILES: c1ccc(-c2nc(-c3ccc4c5ccccc5c5ccccc5c4c3)nc(-c3cccc4c3ccc3oc5ccccc5c34)n2)cc1 ; c1ccc(-c2nc(-c3ccc4c(ccc5oc6ccccc6c54)c3)nc(-c3ccc4ccc5ccccc5c4c3)n2)cc1 ; c1ccc(-c2nc(-c3ccc4ccc5oc6ccccc6c5c4c3)nc(-c3cccc4c3ccc3ccccc34)n2)cc1 ; c1ccc(-c2nc(-c3cc4ccccc4c4ccccc34)nc(-c3cccc4ccc5oc6ccccc6c5c34)n2)cc1]

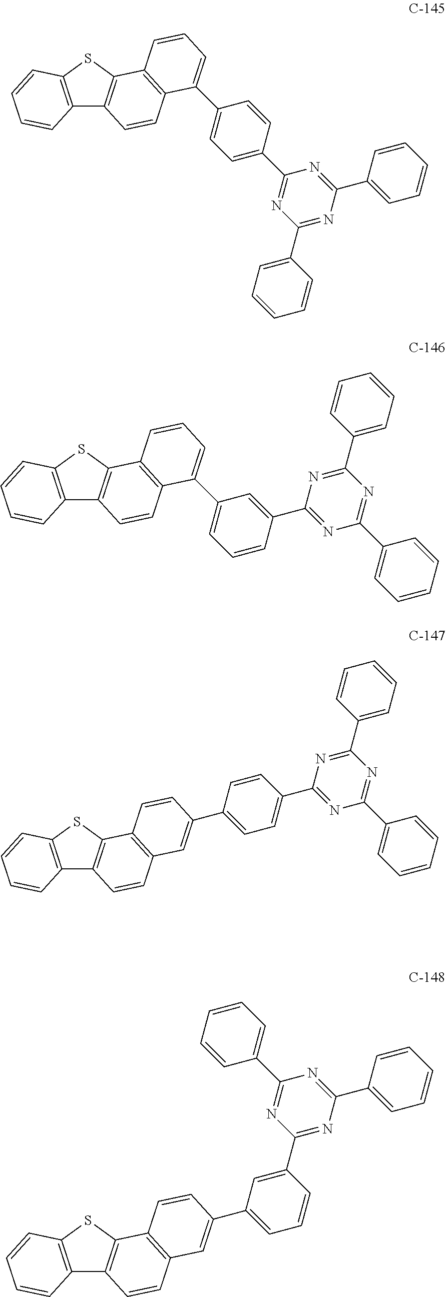 [SMILES: c1ccc(-c2nc(-c3ccccc3)nc(-c3ccc(-c4cccc5c4ccc4c6ccccc6sc54)cc3)n2)cc1 ; c1ccc(-c2nc(-c3ccccc3)nc(-c3cccc(-c4cccc5c4ccc4c6ccccc6sc54)c3)n2)cc1 ; c1ccc(-c2nc(-c3ccccc3)nc(-c3ccc(-c4ccc5c(ccc6c7ccccc7sc56)c4)cc3)n2)cc1 ; c1ccc(-c2nc(-c3ccccc3)nc(-c3cccc(-c4ccc5c(ccc6c7ccccc7sc56)c4)c3)n2)cc1]

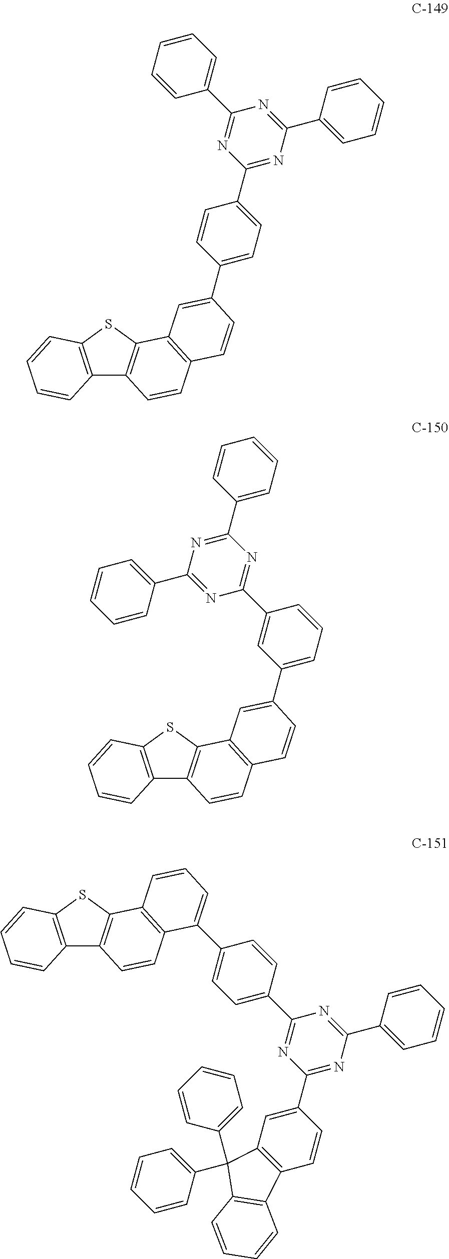 [SMILES: c1ccc(-c2nc(-c3ccccc3)nc(-c3ccc(-c4ccc5ccc6c7ccccc7sc6c5c4)cc3)n2)cc1 ; c1ccc(-c2nc(-c3ccccc3)nc(-c3cccc(-c4ccc5ccc6c7ccccc7sc6c5c4)c3)n2)cc1 ; c1ccc(-c2nc(-c3ccc(-c4cccc5c4ccc4c6ccccc6sc54)cc3)nc(-c3ccc4c(c3)C(c3ccccc3)(c3ccccc3)c3ccccc3-4)n2)cc1]

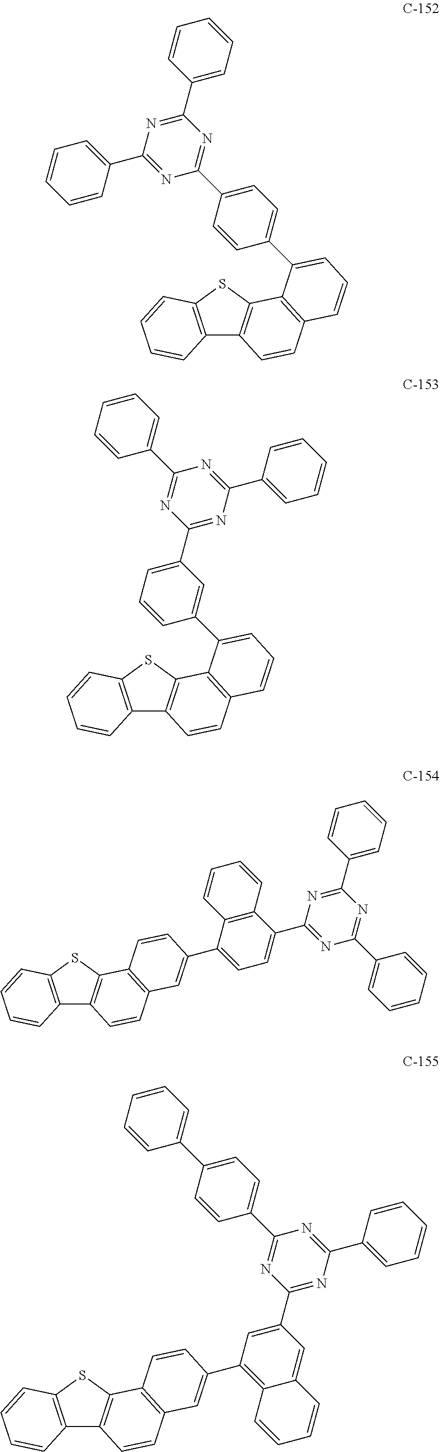

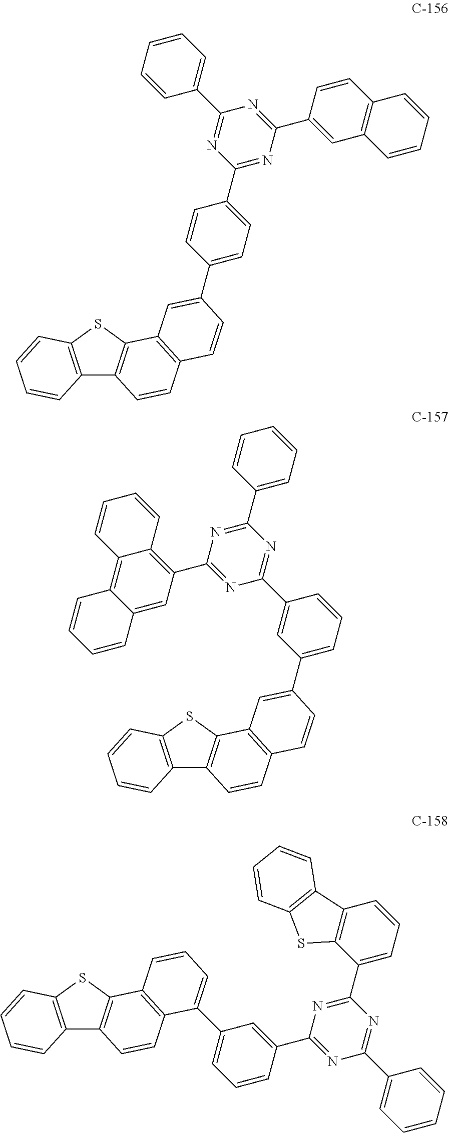 [SMILES: c1ccc(-c2nc(-c3ccc(-c4ccc5ccc6c7ccccc7sc6c5c4)cc3)nc(-c3ccc4ccccc4c3)n2)cc1 ; c1ccc(-c2nc(-c3cccc(-c4ccc5ccc6c7ccccc7sc6c5c4)c3)nc(-c3cc4ccccc4c4ccccc34)n2)cc1 ; c1ccc(-c2nc(-c3cccc(-c4cccc5c4ccc4c6ccccc6sc54)c3)nc(-c3cccc4c3sc3ccccc34)n2)cc1]

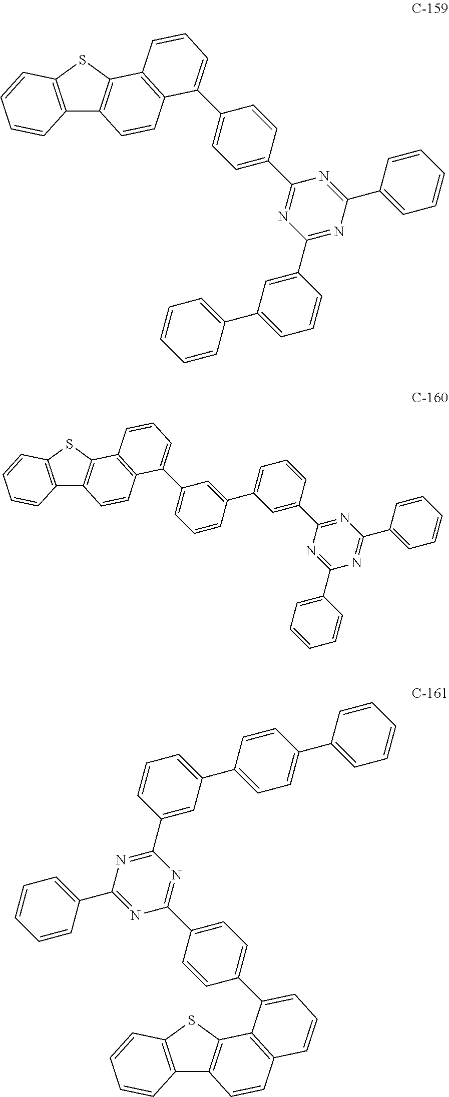 [SMILES: c1ccc(-c2cccc(-c3nc(-c4ccccc4)nc(-c4ccc(-c5cccc6c5ccc5c7ccccc7sc65)cc4)n3)c2)cc1 ; c1ccc(-c2nc(-c3ccccc3)nc(-c3cccc(-c4cccc(-c5cccc6c5ccc5c7ccccc7sc65)c4)c3)n2)cc1 ; c1ccc(-c2ccc(-c3cccc(-c4nc(-c5ccccc5)nc(-c5ccc(-c6cccc7ccc8c9ccccc9sc8c67)cc5)n4)c3)cc2)cc1]

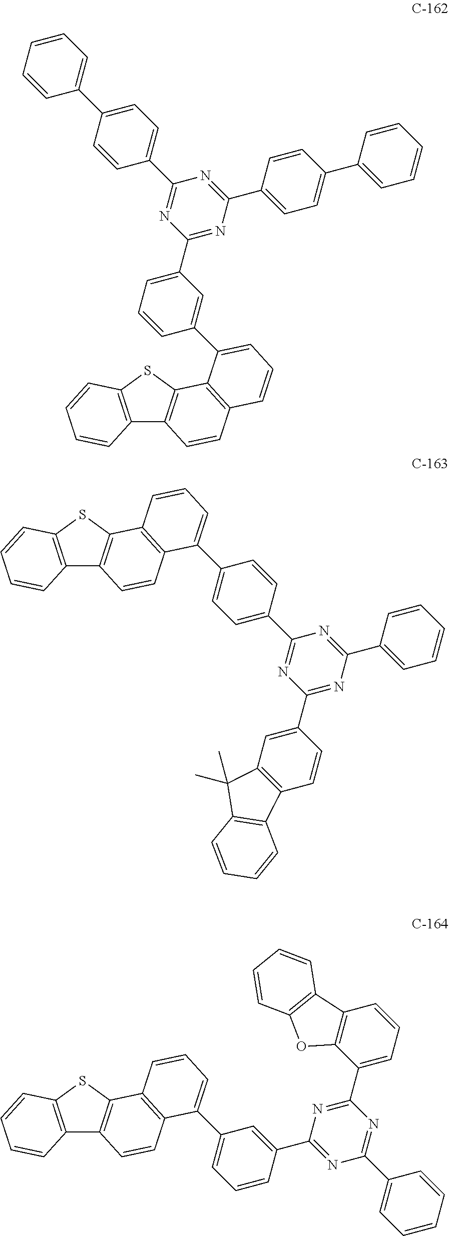

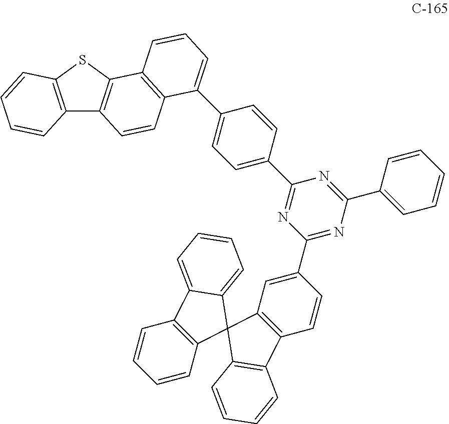 [SMILES: c1ccc(-c2nc(-c3ccc(-c4cccc5c4ccc4c6ccccc6sc54)cc3)nc(-c3ccc4c(c3)C3(c5ccccc5-c5ccccc53)c3ccccc3-4)n2)cc1]

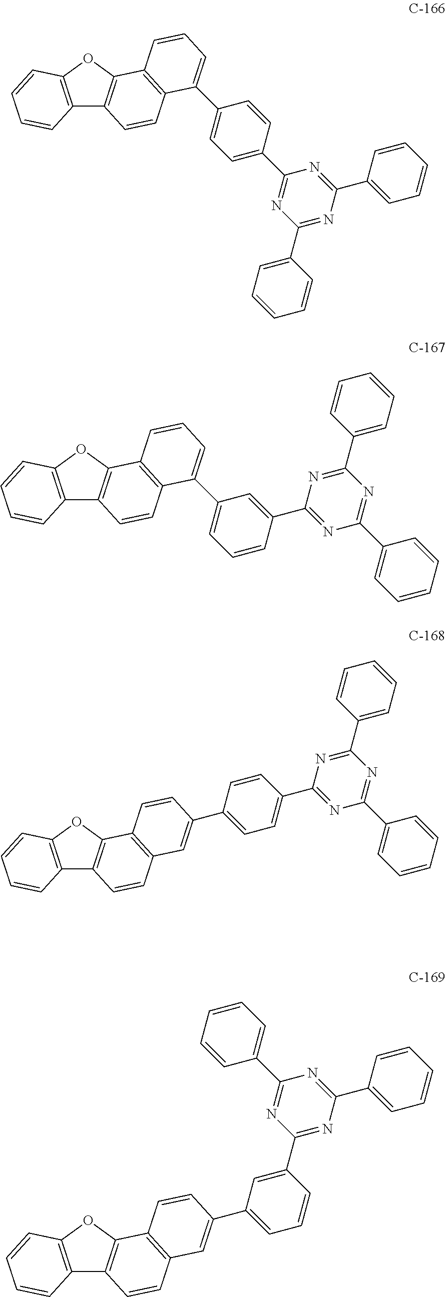 [SMILES: c1ccc(-c2nc(-c3ccccc3)nc(-c3ccc(-c4cccc5c4ccc4c6ccccc6oc54)cc3)n2)cc1 ; c1ccc(-c2nc(-c3ccccc3)nc(-c3cccc(-c4cccc5c4ccc4c6ccccc6oc54)c3)n2)cc1 ; c1ccc(-c2nc(-c3ccccc3)nc(-c3ccc(-c4ccc5c(ccc6c7ccccc7oc56)c4)cc3)n2)cc1 ; c1ccc(-c2nc(-c3ccccc3)nc(-c3cccc(-c4ccc5c(ccc6c7ccccc7oc56)c4)c3)n2)cc1]

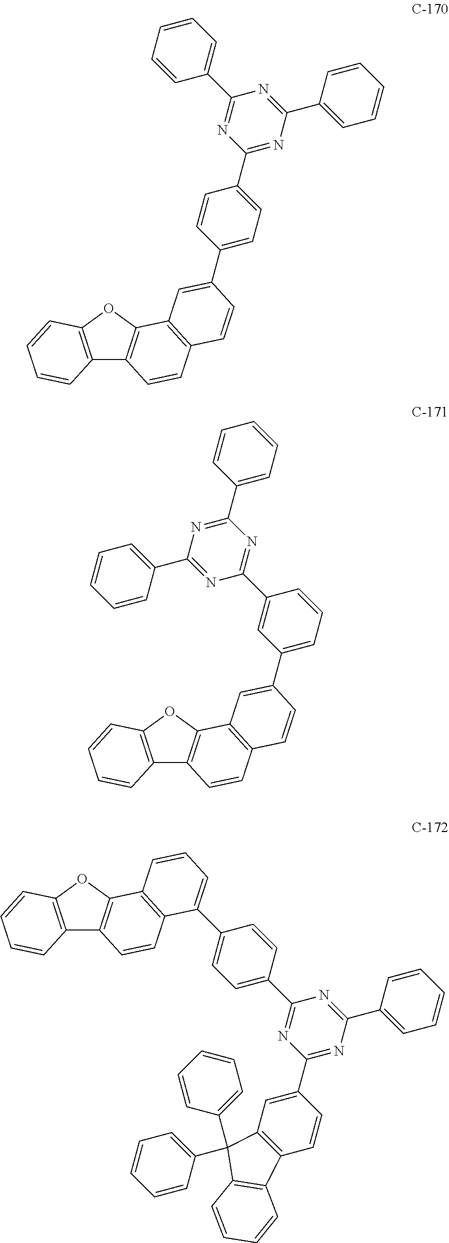 [SMILES: c1ccc(-c2nc(-c3ccccc3)nc(-c3ccc(-c4ccc5ccc6c7ccccc7oc6c5c4)cc3)n2)cc1 ; c1ccc(-c2nc(-c3ccccc3)nc(-c3cccc(-c4ccc5ccc6c7ccccc7oc6c5c4)c3)n2)cc1 ; c1ccc(-c2nc(-c3ccc(-c4cccc5c4ccc4c6ccccc6oc54)cc3)nc(-c3ccc4c(c3)C(c3ccccc3)(c3ccccc3)c3ccccc3-4)n2)cc1]

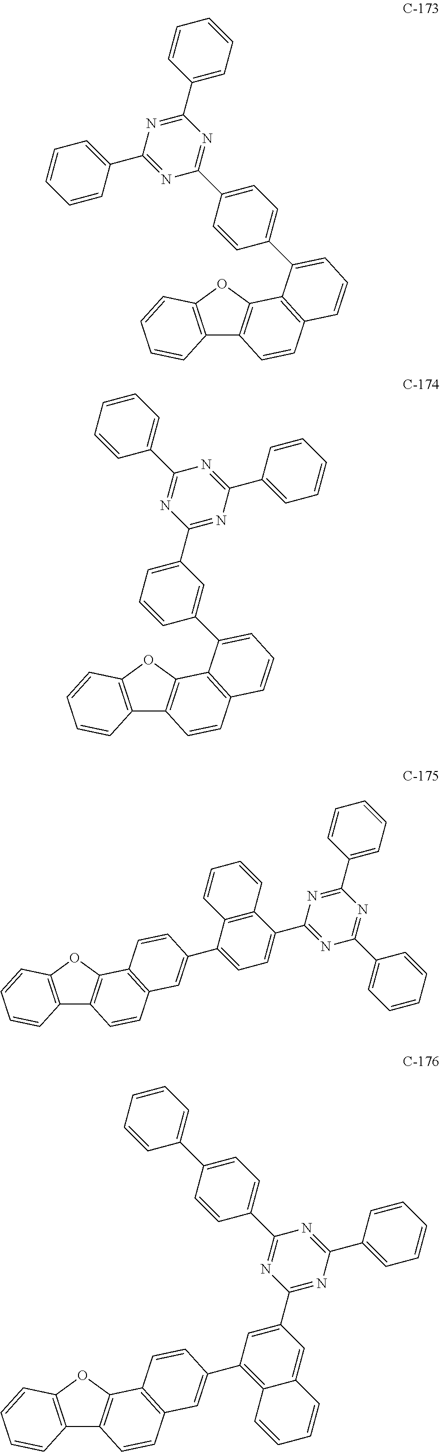

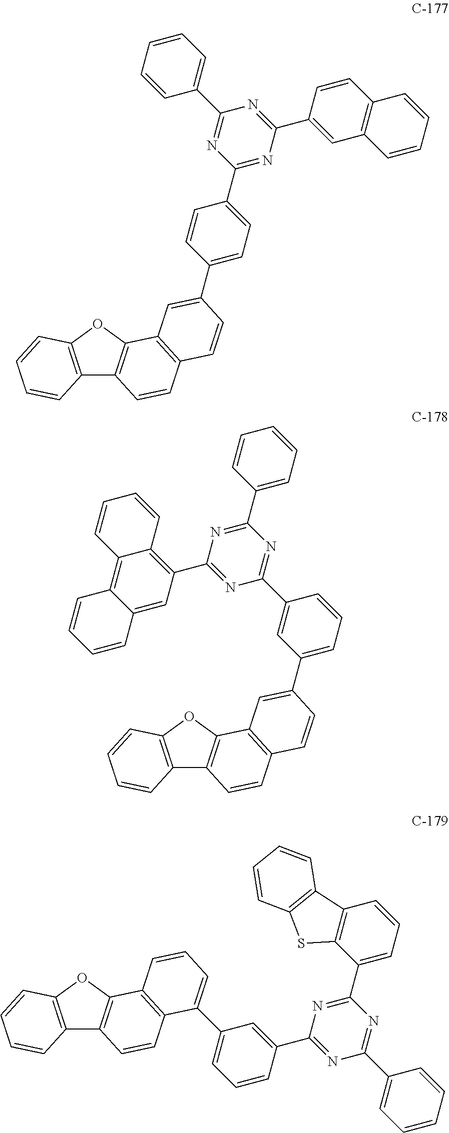 [SMILES: c1ccc(-c2nc(-c3ccc(-c4ccc5ccc6c7ccccc7oc6c5c4)cc3)nc(-c3ccc4ccccc4c3)n2)cc1 ; c1ccc(-c2nc(-c3cccc(-c4ccc5ccc6c7ccccc7oc6c5c4)c3)nc(-c3cc4ccccc4c4ccccc34)n2)cc1 ; c1ccc(-c2nc(-c3cccc(-c4cccc5c4ccc4c6ccccc6oc54)c3)nc(-c3cccc4c3sc3ccccc34)n2)cc1]

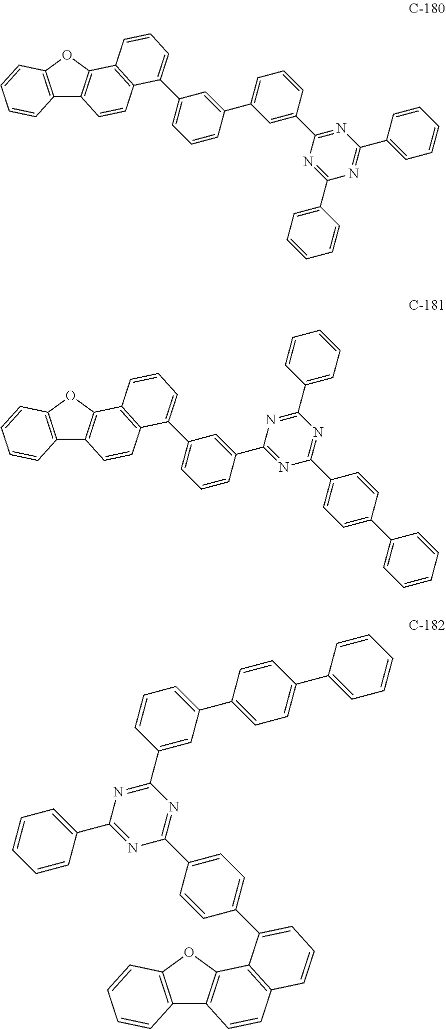 [SMILES: c1ccc(-c2nc(-c3ccccc3)nc(-c3cccc(-c4cccc(-c5cccc6c5ccc5c7ccccc7oc65)c4)c3)n2)cc1 ; c1ccc(-c2ccc(-c3nc(-c4ccccc4)nc(-c4cccc(-c5cccc6c5ccc5c7ccccc7oc65)c4)n3)cc2)cc1 ; c1ccc(-c2ccc(-c3cccc(-c4nc(-c5ccccc5)nc(-c5ccc(-c6cccc7ccc8c9ccccc9oc8c67)cc5)n4)c3)cc2)cc1]

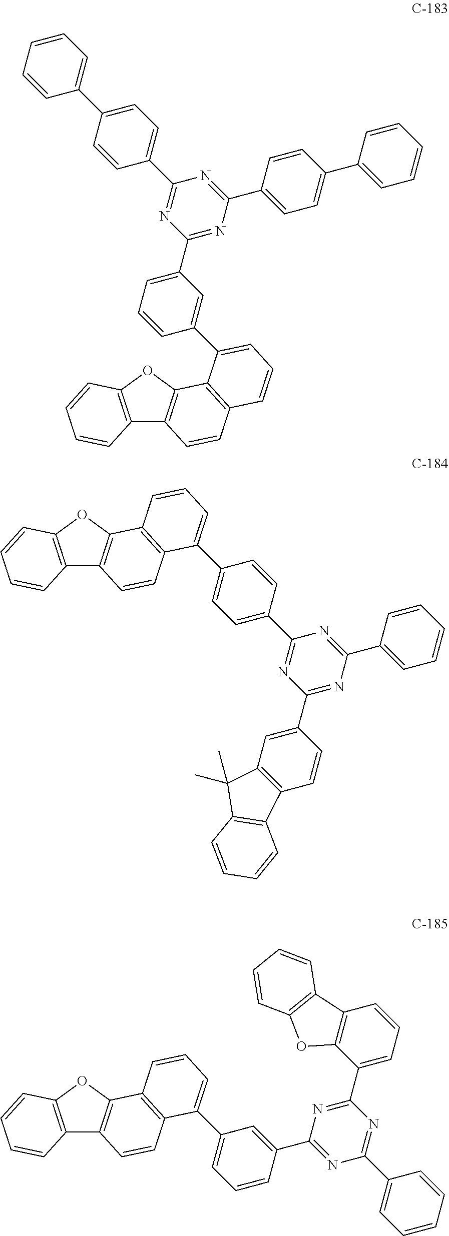

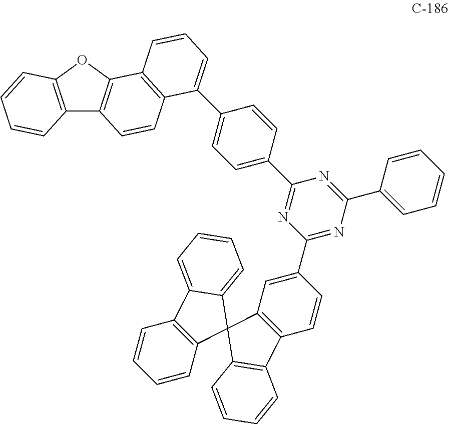 [SMILES: c1ccc(-c2nc(-c3ccc(-c4cccc5c4ccc4c6ccccc6oc54)cc3)nc(-c3ccc4c(c3)C3(c5ccccc5-c5ccccc53)c3ccccc3-4)n2)cc1]

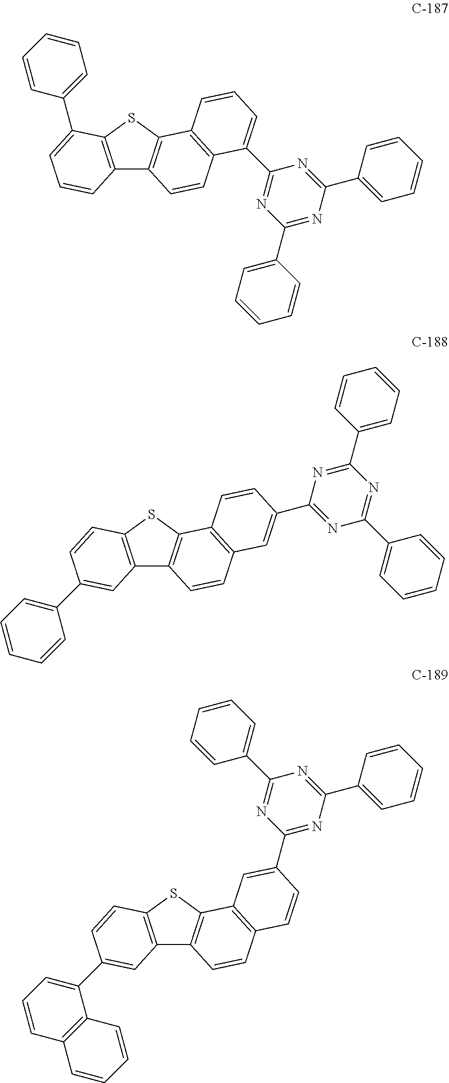 [SMILES: c1ccc(-c2nc(-c3ccccc3)nc(-c3cccc4c3ccc3c5cccc(-c6ccccc6)c5sc43)n2)cc1 ; c1ccc(-c2ccc3sc4c5ccc(-c6nc(-c7ccccc7)nc(-c7ccccc7)n6)cc5ccc4c3c2)cc1 ; c1ccc(-c2nc(-c3ccccc3)nc(-c3ccc4ccc5c6cc(-c7cccc8ccccc78)ccc6sc5c4c3)n2)cc1]

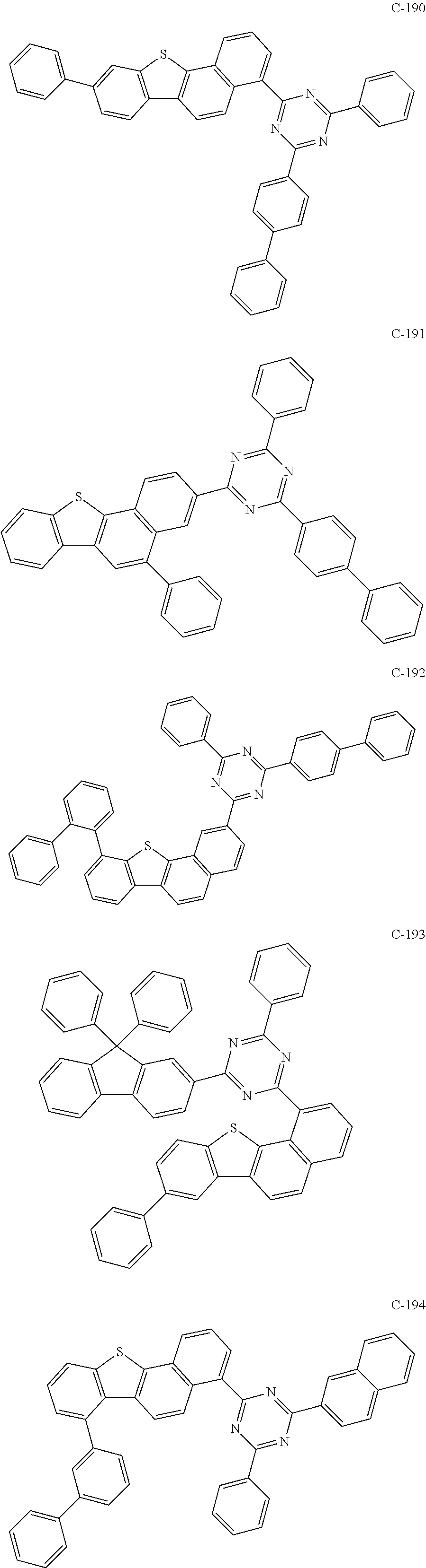 [SMILES: c1ccc(-c2ccc(-c3nc(-c4ccccc4)nc(-c4cccc5c4ccc4c6ccc(-c7ccccc7)cc6sc54)n3)cc2)cc1 ; c1ccc(-c2ccc(-c3nc(-c4ccccc4)nc(-c4ccc5c(c4)c(-c4ccccc4)cc4c6ccccc6sc54)n3)cc2)cc1 ; c1ccc(-c2ccc(-c3nc(-c4ccccc4)nc(-c4ccc5ccc6c7cccc(-c8ccccc8-c8ccccc8)c7sc6c5c4)n3)cc2)cc1 ; c1ccc(-c2ccc3sc4c(ccc5cccc(-c6nc(-c7ccccc7)nc(-c7ccc8c(c7)C(c7ccccc7)(c7ccccc7)c7ccccc7-8)n6)c54)c3c2)cc1 ; c1ccc(-c2cccc(-c3cccc4sc5c6cccc(-c7nc(-c8ccccc8)nc(-c8ccc9ccccc9c8)n7)c6ccc5c34)c2)cc1]

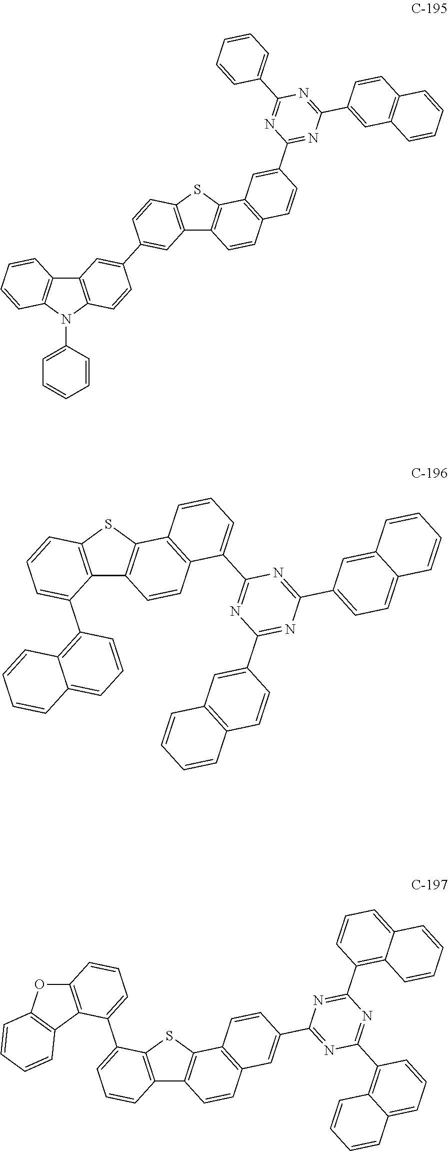 [SMILES: c1ccc(-c2nc(-c3ccc4ccccc4c3)nc(-c3ccc4ccc5c6cc(-c7ccc8c(c7)c7ccccc7n8-c7ccccc7)ccc6sc5c4c3)n2)cc1 ; c1ccc2cc(-c3nc(-c4ccc5ccccc5c4)nc(-c4cccc5c4ccc4c5sc5cccc(-c6cccc7ccccc67)c54)n3)ccc2c1 ; c1ccc2c(-c3nc(-c4ccc5c(ccc6c7cccc(-c8cccc9oc%10ccccc%10c89)c7sc56)c4)nc(-c4cccc5ccccc45)n3)cccc2c1]

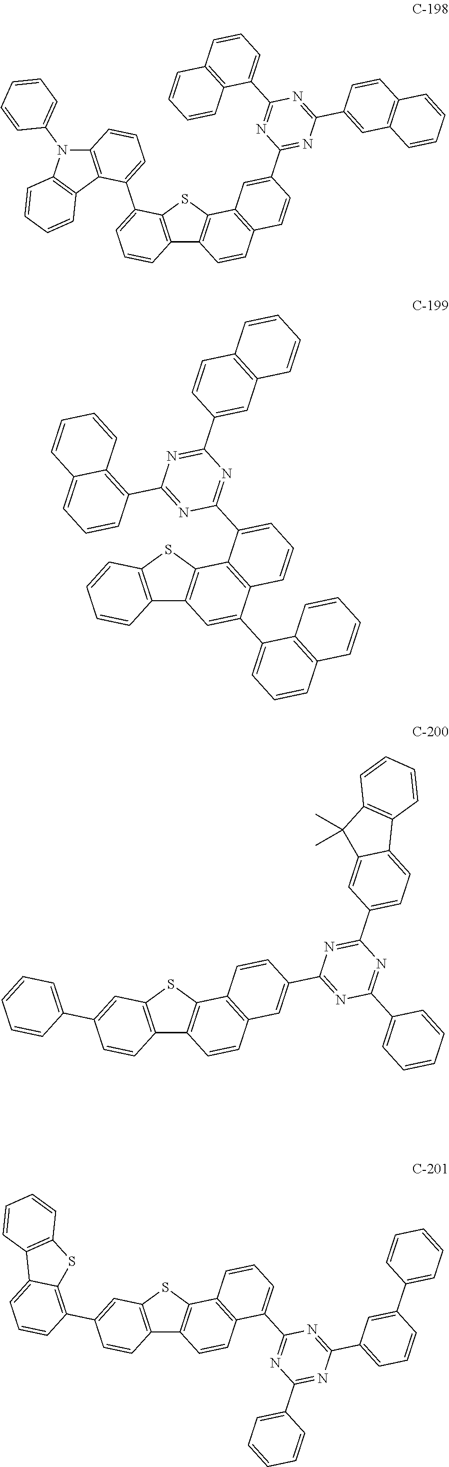 [SMILES: c1ccc(-n2c3ccccc3c3c(-c4cccc5c4sc4c6cc(-c7nc(-c8ccc9ccccc9c8)nc(-c8cccc9ccccc89)n7)ccc6ccc54)cccc32)cc1 ; c1ccc2cc(-c3nc(-c4cccc5ccccc45)nc(-c4cccc5c(-c6cccc7ccccc67)cc6c7ccccc7sc6c45)n3)ccc2c1 ; CC1(C)c2ccccc2-c2ccc(-c3nc(-c4ccccc4)nc(-c4ccc5c(ccc6c7ccc(-c8ccccc8)cc7sc56)c4)n3)cc21 ; c1ccc(-c2cccc(-c3nc(-c4ccccc4)nc(-c4cccc5c4ccc4c6ccc(-c7cccc8c7sc7ccccc78)cc6sc54)n3)c2)cc1]

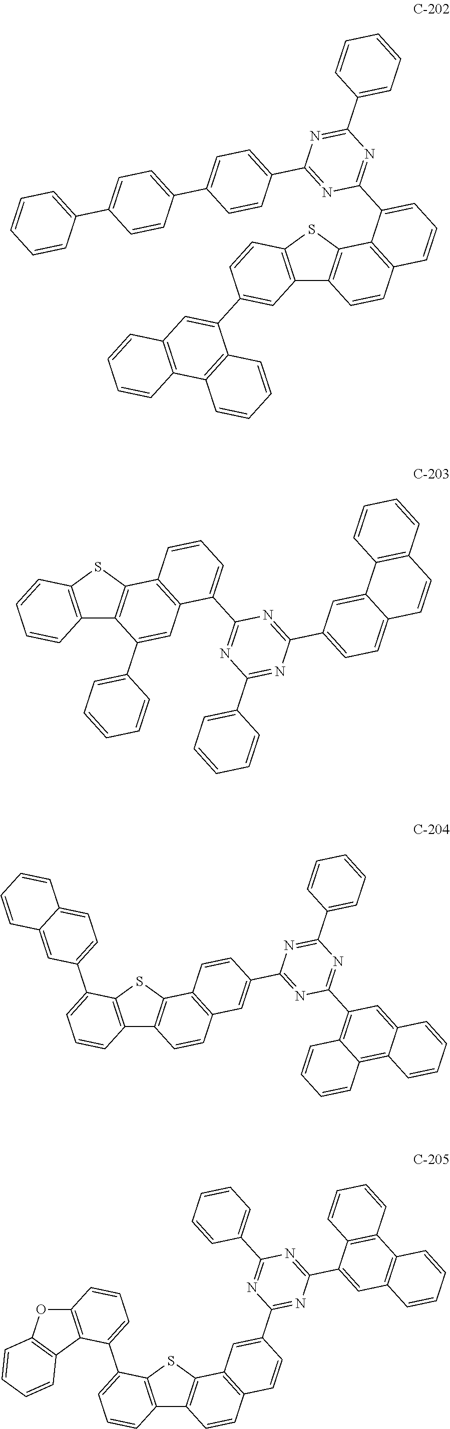 [SMILES: c1ccc(-c2ccc(-c3ccc(-c4nc(-c5ccccc5)nc(-c5cccc6ccc7c8cc(-c9cc%10ccccc%10c%10ccccc9%10)ccc8sc7c56)n4)cc3)cc2)cc1 ; c1ccc(-c2nc(-c3ccc4ccc5ccccc5c4c3)nc(-c3cccc4c3cc(-c3ccccc3)c3c5ccccc5sc43)n2)cc1 ; c1ccc(-c2nc(-c3ccc4c(ccc5c6cccc(-c7ccc8ccccc8c7)c6sc45)c3)nc(-c3cc4ccccc4c4ccccc34)n2)cc1 ; c1ccc(-c2nc(-c3ccc4ccc5c6cccc(-c7cccc8oc9ccccc9c78)c6sc5c4c3)nc(-c3cc4ccccc4c4ccccc34)n2)cc1]

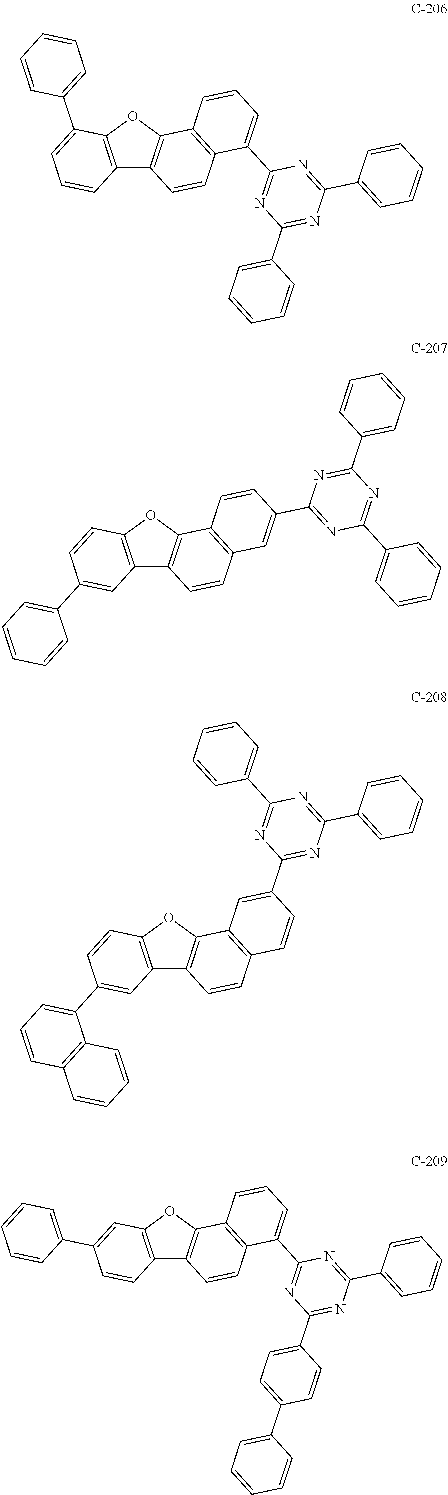 [SMILES: c1ccc(-c2nc(-c3ccccc3)nc(-c3cccc4c3ccc3c5cccc(-c6ccccc6)c5oc43)n2)cc1 ; c1ccc(-c2ccc3oc4c5ccc(-c6nc(-c7ccccc7)nc(-c7ccccc7)n6)cc5ccc4c3c2)cc1 ; c1ccc(-c2nc(-c3ccccc3)nc(-c3ccc4ccc5c6cc(-c7cccc8ccccc78)ccc6oc5c4c3)n2)cc1 ; c1ccc(-c2ccc(-c3nc(-c4ccccc4)nc(-c4cccc5c4ccc4c6ccc(-c7ccccc7)cc6oc54)n3)cc2)cc1]

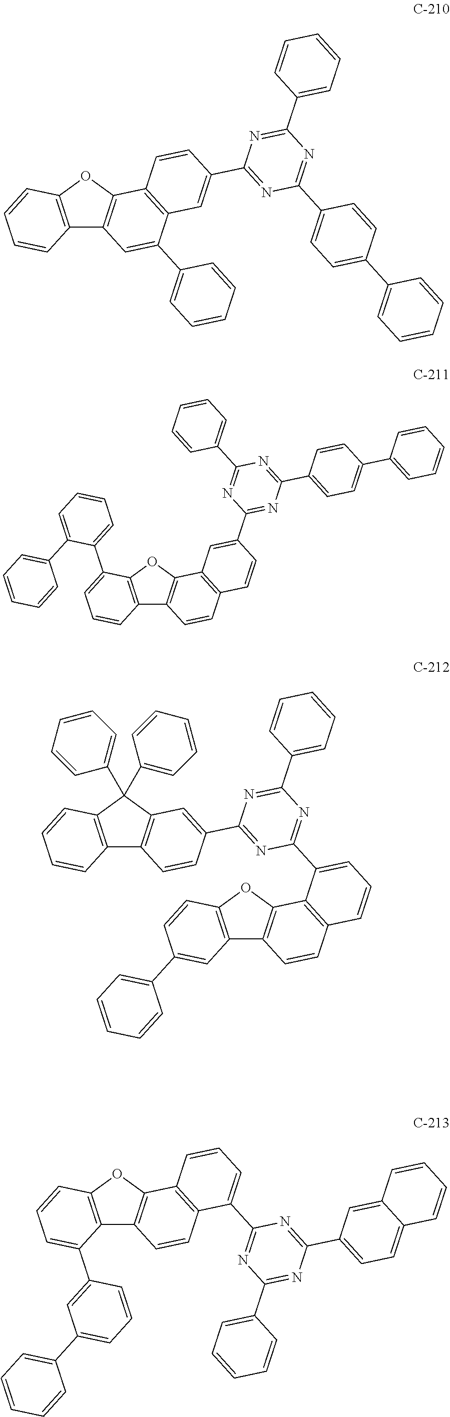 [SMILES: c1ccc(-c2ccc(-c3nc(-c4ccccc4)nc(-c4ccc5c(c4)c(-c4ccccc4)cc4c6ccccc6oc54)n3)cc2)cc1 ; c1ccc(-c2ccc(-c3nc(-c4ccccc4)nc(-c4ccc5ccc6c7cccc(-c8ccccc8-c8ccccc8)c7oc6c5c4)n3)cc2)cc1 ; c1ccc(-c2ccc3oc4c(ccc5cccc(-c6nc(-c7ccccc7)nc(-c7ccc8c(c7)C(c7ccccc7)(c7ccccc7)c7ccccc7-8)n6)c54)c3c2)cc1 ; c1ccc(-c2cccc(-c3cccc4oc5c6cccc(-c7nc(-c8ccccc8)nc(-c8ccc9ccccc9c8)n7)c6ccc5c34)c2)cc1]

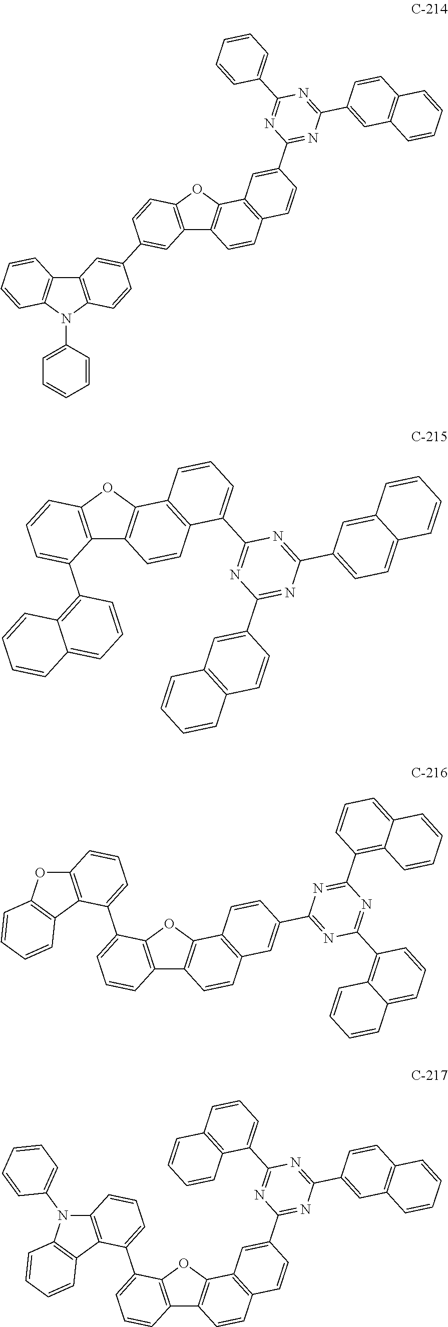 [SMILES: c1ccc(-c2nc(-c3ccc4ccccc4c3)nc(-c3ccc4ccc5c6cc(-c7ccc8c(c7)c7ccccc7n8-c7ccccc7)ccc6oc5c4c3)n2)cc1 ; c1ccc2cc(-c3nc(-c4ccc5ccccc5c4)nc(-c4cccc5c4ccc4c5oc5cccc(-c6cccc7ccccc67)c54)n3)ccc2c1 ; c1ccc2c(-c3nc(-c4ccc5c(ccc6c7cccc(-c8cccc9oc%10ccccc%10c89)c7oc56)c4)nc(-c4cccc5ccccc45)n3)cccc2c1 ; c1ccc(-n2c3ccccc3c3c(-c4cccc5c4oc4c6cc(-c7nc(-c8ccc9ccccc9c8)nc(-c8cccc9ccccc89)n7)ccc6ccc54)cccc32)cc1]

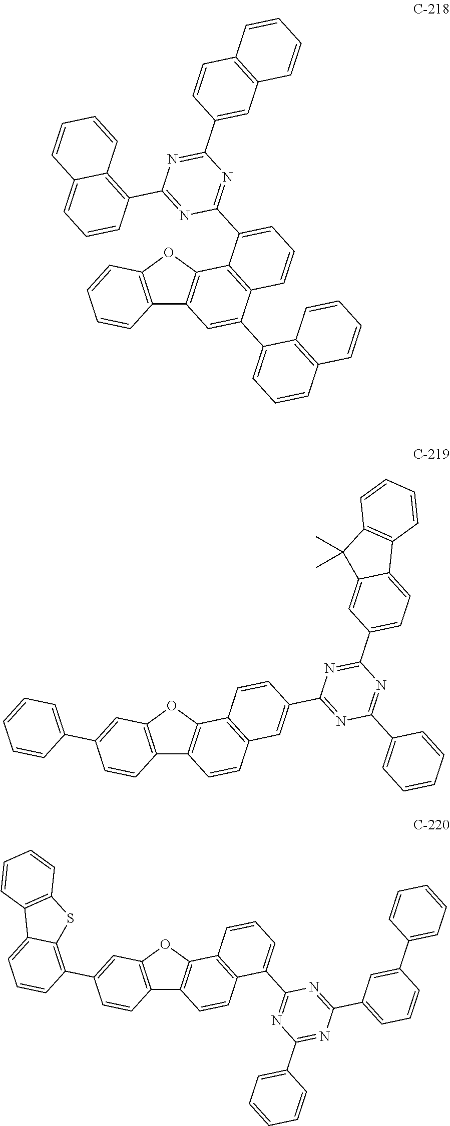 [SMILES: c1ccc2cc(-c3nc(-c4cccc5ccccc45)nc(-c4cccc5c(-c6cccc7ccccc67)cc6c7ccccc7oc6c45)n3)ccc2c1 ; CC1(C)c2ccccc2-c2ccc(-c3nc(-c4ccccc4)nc(-c4ccc5c(ccc6c7ccc(-c8ccccc8)cc7oc56)c4)n3)cc21 ; c1ccc(-c2cccc(-c3nc(-c4ccccc4)nc(-c4cccc5c4ccc4c6ccc(-c7cccc8c7sc7ccccc78)cc6oc54)n3)c2)cc1]

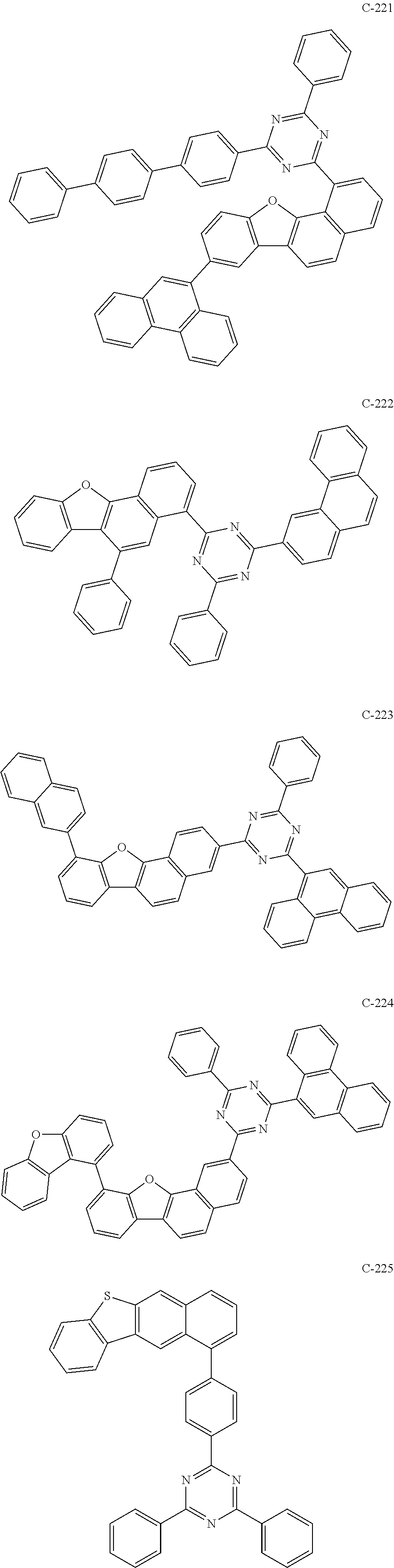 [SMILES: c1ccc(-c2ccc(-c3ccc(-c4nc(-c5ccccc5)nc(-c5cccc6ccc7c8cc(-c9cc%10ccccc%10c%10ccccc9%10)ccc8oc7c56)n4)cc3)cc2)cc1 ; c1ccc(-c2nc(-c3ccc4ccc5ccccc5c4c3)nc(-c3cccc4c3cc(-c3ccccc3)c3c5ccccc5oc43)n2)cc1 ; c1ccc(-c2nc(-c3ccc4c(ccc5c6cccc(-c7ccc8ccccc8c7)c6oc45)c3)nc(-c3cc4ccccc4c4ccccc34)n2)cc1 ; c1ccc(-c2nc(-c3ccc4ccc5c6cccc(-c7cccc8oc9ccccc9c78)c6oc5c4c3)nc(-c3cc4ccccc4c4ccccc34)n2)cc1 ; c1ccc(-c2nc(-c3ccccc3)nc(-c3ccc(-c4cccc5cc6sc7ccccc7c6cc45)cc3)n2)cc1]

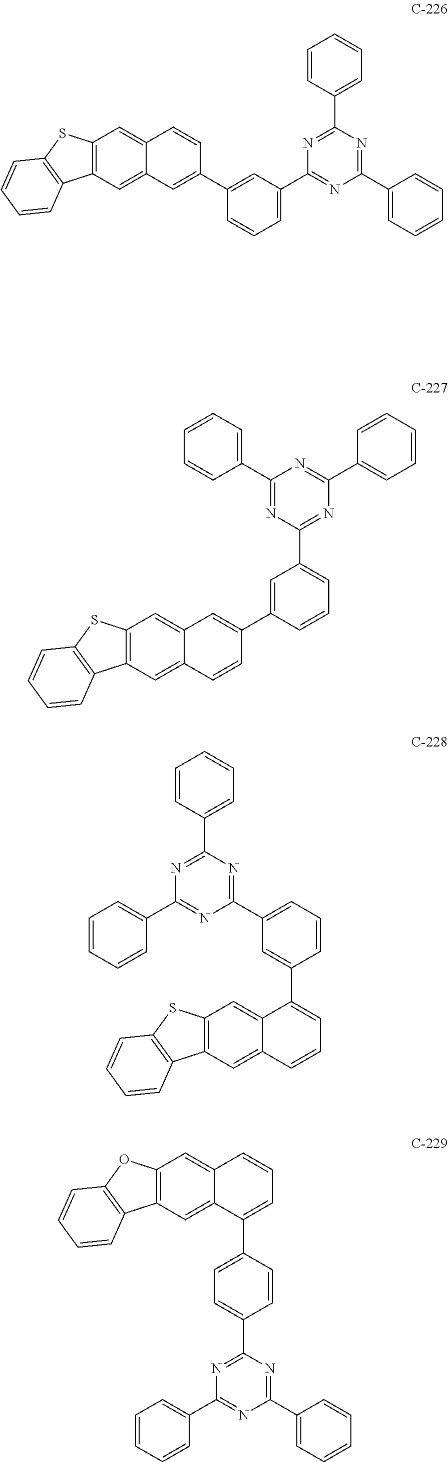 [SMILES: c1ccc(-c2nc(-c3ccccc3)nc(-c3cccc(-c4ccc5cc6sc7ccccc7c6cc5c4)c3)n2)cc1 ; c1ccc(-c2nc(-c3ccccc3)nc(-c3cccc(-c4ccc5cc6c(cc5c4)sc4ccccc46)c3)n2)cc1 ; c1ccc(-c2nc(-c3ccccc3)nc(-c3cccc(-c4cccc5cc6c(cc45)sc4ccccc46)c3)n2)cc1 ; c1ccc(-c2nc(-c3ccccc3)nc(-c3ccc(-c4cccc5cc6oc7ccccc7c6cc45)cc3)n2)cc1]

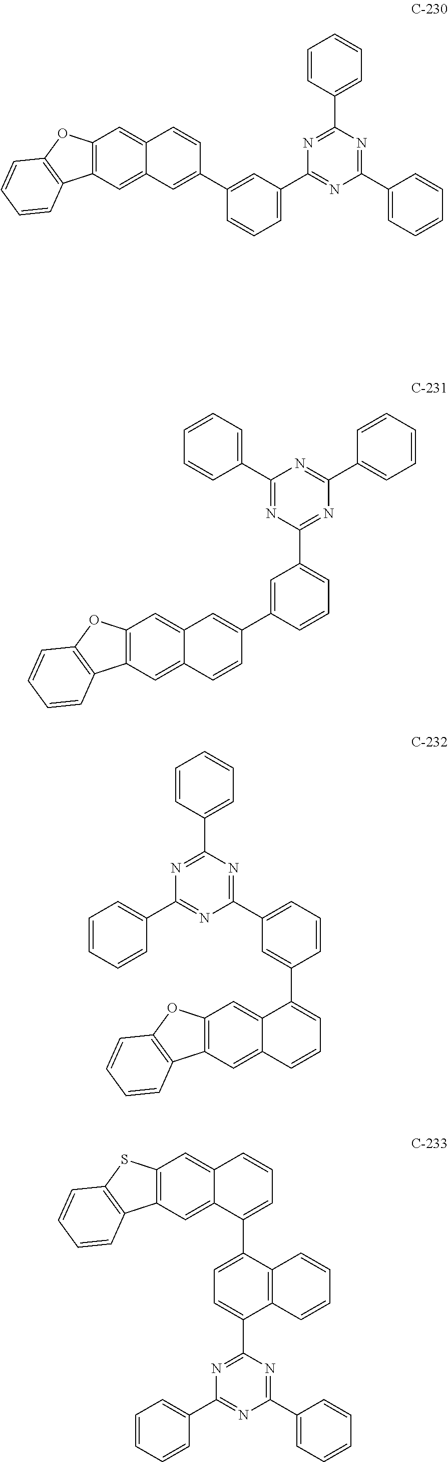 [SMILES: c1ccc(-c2nc(-c3ccccc3)nc(-c3cccc(-c4ccc5cc6oc7ccccc7c6cc5c4)c3)n2)cc1 ; c1ccc(-c2nc(-c3ccccc3)nc(-c3cccc(-c4ccc5cc6c(cc5c4)oc4ccccc46)c3)n2)cc1 ; c1ccc(-c2nc(-c3ccccc3)nc(-c3cccc(-c4cccc5cc6c(cc45)oc4ccccc46)c3)n2)cc1 ; c1ccc(-c2nc(-c3ccccc3)nc(-c3ccc(-c4cccc5cc6sc7ccccc7c6cc45)c4ccccc34)n2)cc1]

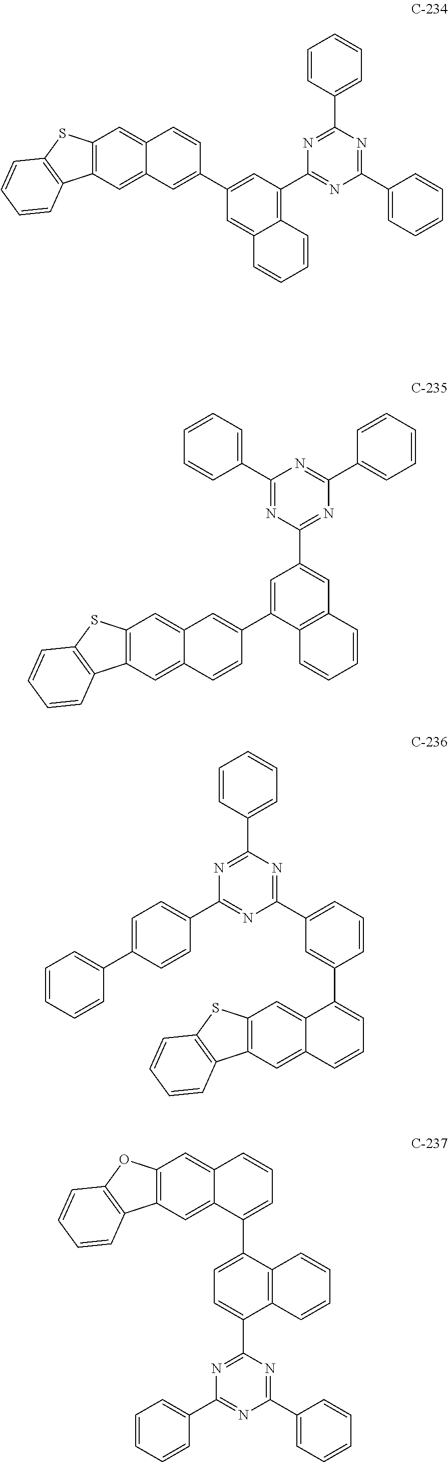 [SMILES: c1ccc(-c2nc(-c3ccccc3)nc(-c3cc(-c4ccc5cc6sc7ccccc7c6cc5c4)cc4ccccc34)n2)cc1 ; c1ccc(-c2nc(-c3ccccc3)nc(-c3cc(-c4ccc5cc6c(cc5c4)sc4ccccc46)c4ccccc4c3)n2)cc1 ; c1ccc(-c2ccc(-c3nc(-c4ccccc4)nc(-c4cccc(-c5cccc6cc7c(cc56)sc5ccccc57)c4)n3)cc2)cc1 ; c1ccc(-c2nc(-c3ccccc3)nc(-c3ccc(-c4cccc5cc6oc7ccccc7c6cc45)c4ccccc34)n2)cc1]

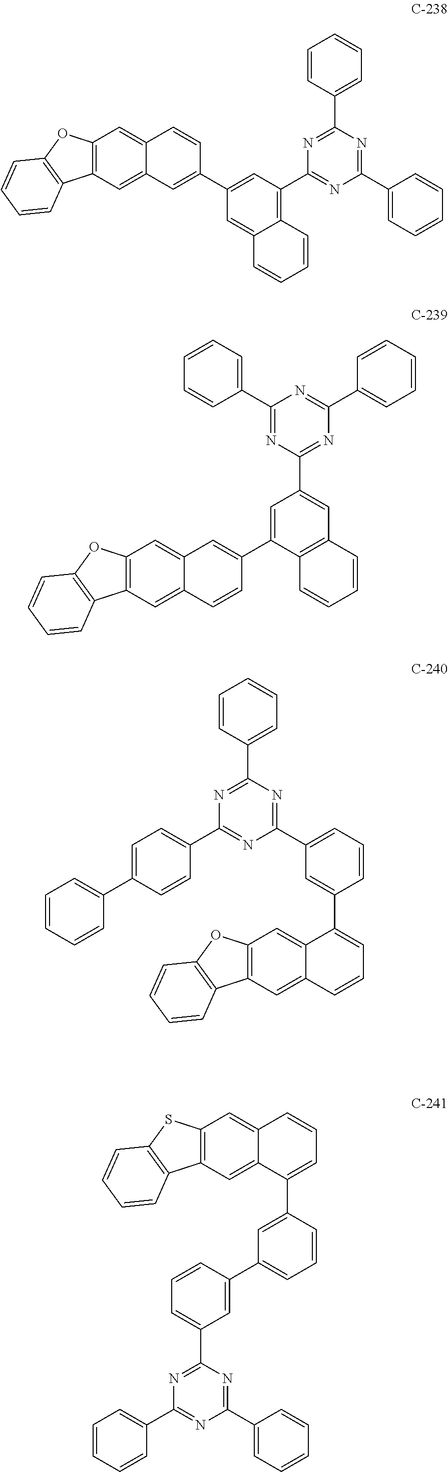 [SMILES: c1ccc(-c2nc(-c3ccccc3)nc(-c3cc(-c4ccc5cc6oc7ccccc7c6cc5c4)cc4ccccc34)n2)cc1 ; c1ccc(-c2nc(-c3ccccc3)nc(-c3cc(-c4ccc5cc6c(cc5c4)oc4ccccc46)c4ccccc4c3)n2)cc1 ; c1ccc(-c2ccc(-c3nc(-c4ccccc4)nc(-c4cccc(-c5cccc6cc7c(cc56)oc5ccccc57)c4)n3)cc2)cc1 ; c1ccc(-c2nc(-c3ccccc3)nc(-c3cccc(-c4cccc(-c5cccc6cc7sc8ccccc8c7cc56)c4)c3)n2)cc1]

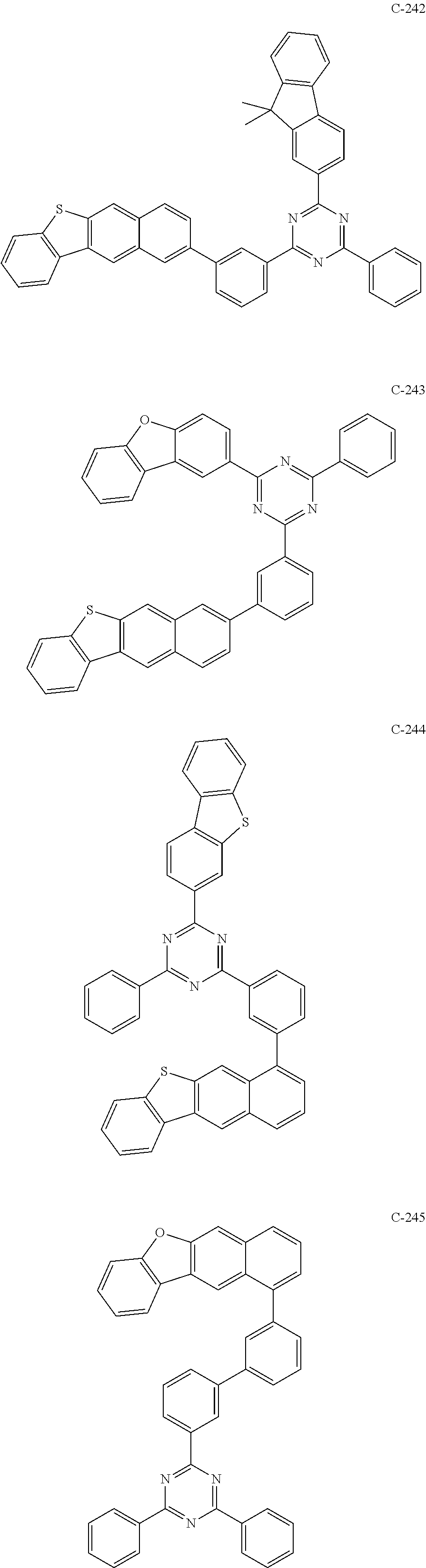 [SMILES: CC1(C)c2ccccc2-c2ccc(-c3nc(-c4ccccc4)nc(-c4cccc(-c5ccc6cc7sc8ccccc8c7cc6c5)c4)n3)cc21 ; c1ccc(-c2nc(-c3cccc(-c4ccc5cc6c(cc5c4)sc4ccccc46)c3)nc(-c3ccc4oc5ccccc5c4c3)n2)cc1 ; c1ccc(-c2nc(-c3cccc(-c4cccc5cc6c(cc45)sc4ccccc46)c3)nc(-c3ccc4c(c3)sc3ccccc34)n2)cc1 ; c1ccc(-c2nc(-c3ccccc3)nc(-c3cccc(-c4cccc(-c5cccc6cc7oc8ccccc8c7cc56)c4)c3)n2)cc1]

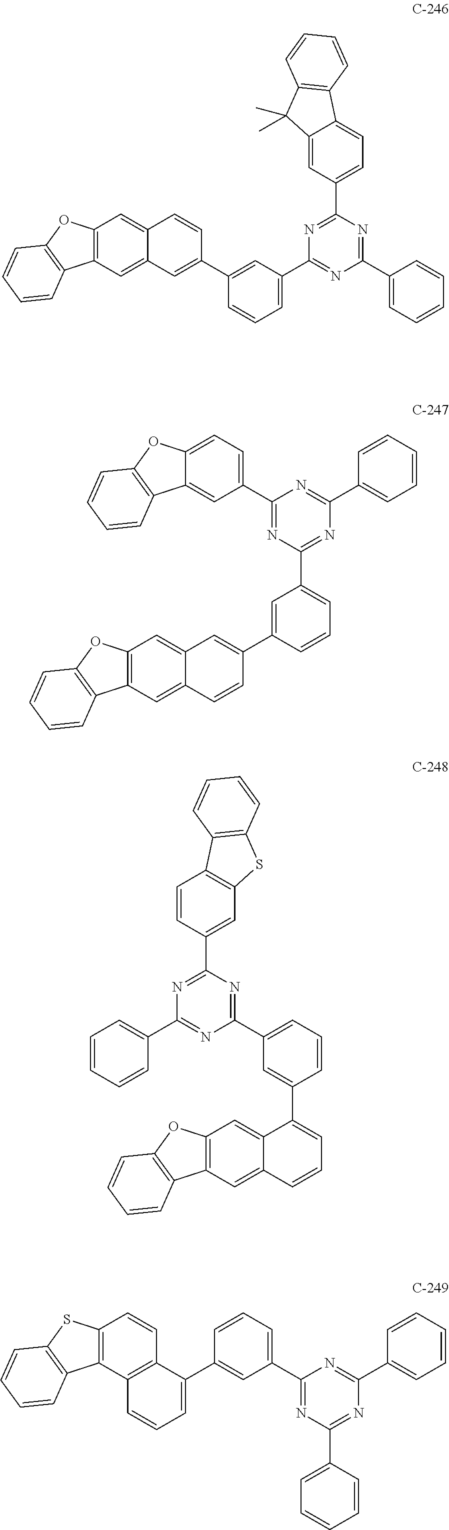 [SMILES: CC1(C)c2ccccc2-c2ccc(-c3nc(-c4ccccc4)nc(-c4cccc(-c5ccc6cc7oc8ccccc8c7cc6c5)c4)n3)cc21 ; c1ccc(-c2nc(-c3cccc(-c4ccc5cc6c(cc5c4)oc4ccccc46)c3)nc(-c3ccc4oc5ccccc5c4c3)n2)cc1 ; c1ccc(-c2nc(-c3cccc(-c4cccc5cc6c(cc45)oc4ccccc46)c3)nc(-c3ccc4c(c3)sc3ccccc34)n2)cc1 ; c1ccc(-c2nc(-c3ccccc3)nc(-c3cccc(-c4cccc5c4ccc4sc6ccccc6c45)c3)n2)cc1]

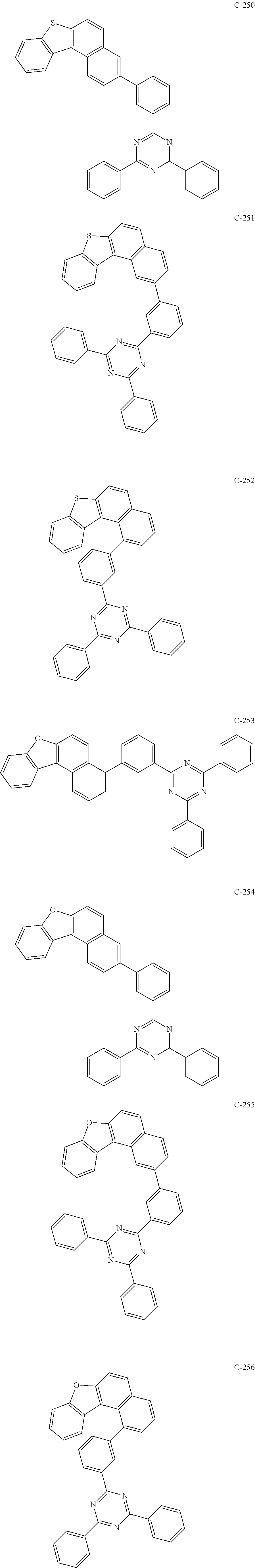

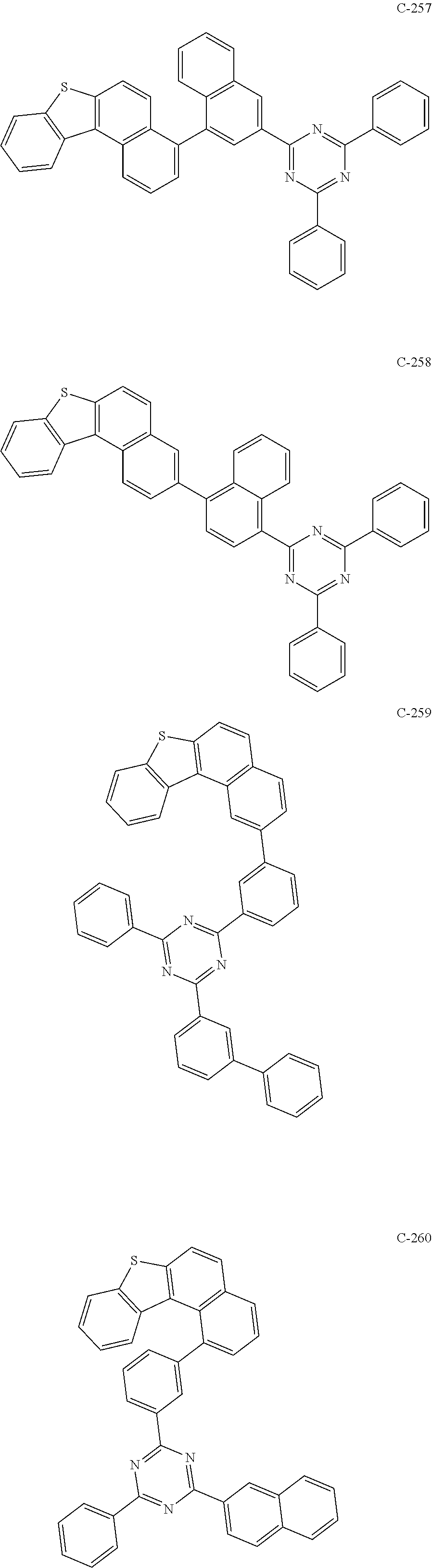 [SMILES: c1ccc(-c2nc(-c3ccccc3)nc(-c3cc(-c4cccc5c4ccc4sc6ccccc6c45)c4ccccc4c3)n2)cc1 ; c1ccc(-c2nc(-c3ccccc3)nc(-c3ccc(-c4ccc5c(ccc6sc7ccccc7c65)c4)c4ccccc34)n2)cc1 ; c1ccc(-c2cccc(-c3nc(-c4ccccc4)nc(-c4cccc(-c5ccc6ccc7sc8ccccc8c7c6c5)c4)n3)c2)cc1 ; c1ccc(-c2nc(-c3cccc(-c4cccc5ccc6sc7ccccc7c6c45)c3)nc(-c3ccc4ccccc4c3)n2)cc1]

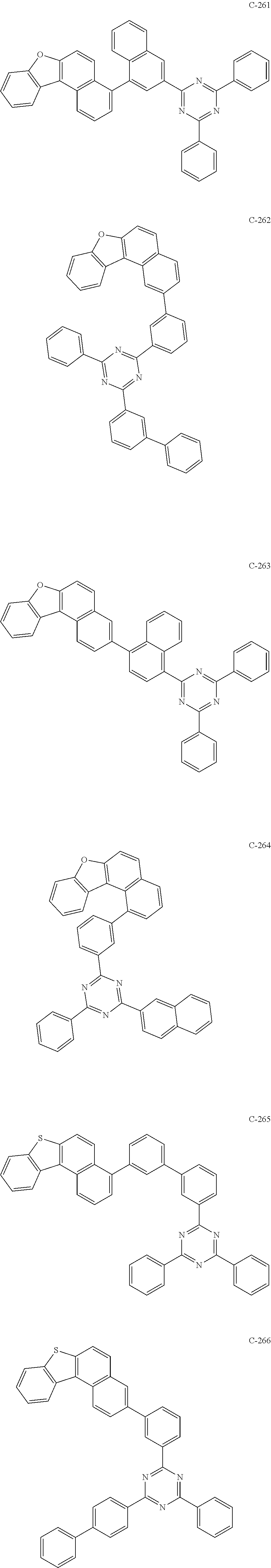 [SMILES: c1ccc(-c2nc(-c3ccccc3)nc(-c3cc(-c4cccc5c4ccc4oc6ccccc6c45)c4ccccc4c3)n2)cc1 ; c1ccc(-c2cccc(-c3nc(-c4ccccc4)nc(-c4cccc(-c5ccc6ccc7oc8ccccc8c7c6c5)c4)n3)c2)cc1 ; c1ccc(-c2nc(-c3ccccc3)nc(-c3ccc(-c4ccc5c(ccc6oc7ccccc7c65)c4)c4ccccc34)n2)cc1 ; c1ccc(-c2nc(-c3cccc(-c4cccc5ccc6oc7ccccc7c6c45)c3)nc(-c3ccc4ccccc4c3)n2)cc1 ; c1ccc(-c2nc(-c3ccccc3)nc(-c3cccc(-c4cccc(-c5cccc6c5ccc5sc7ccccc7c56)c4)c3)n2)cc1 ; c1ccc(-c2ccc(-c3nc(-c4ccccc4)nc(-c4cccc(-c5ccc6c(ccc7sc8ccccc8c76)c5)c4)n3)cc2)cc1]

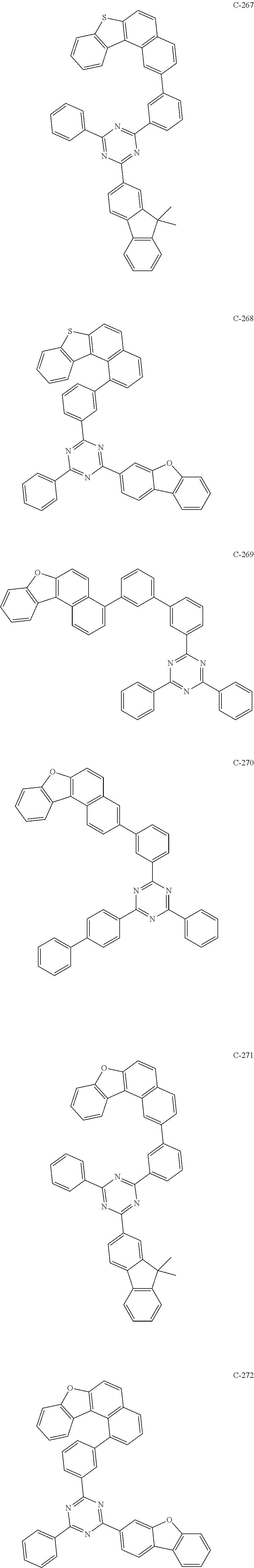

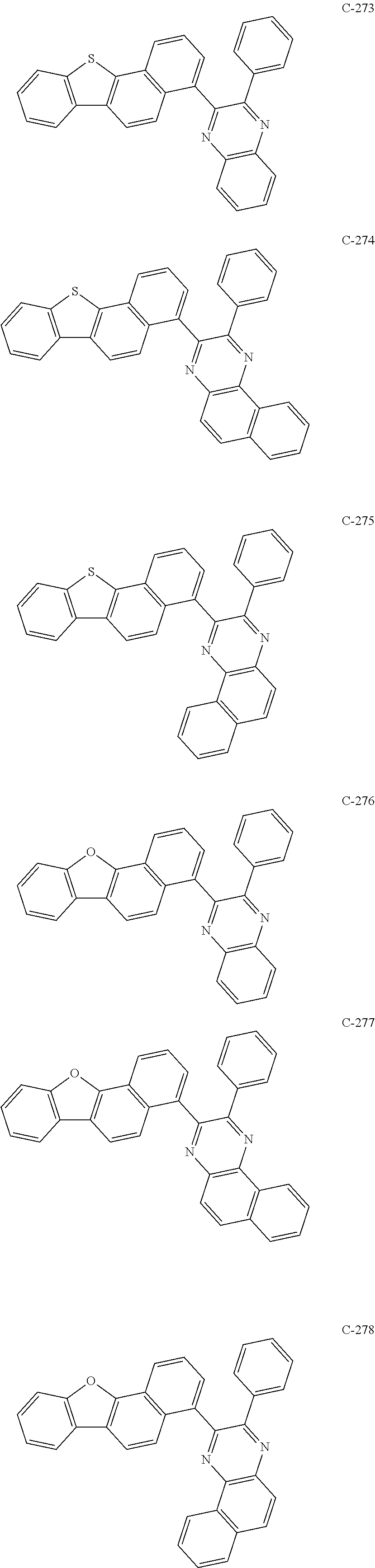

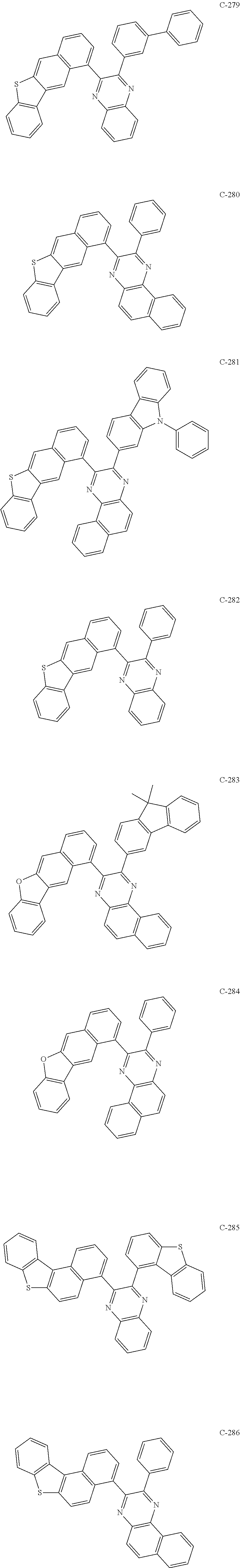

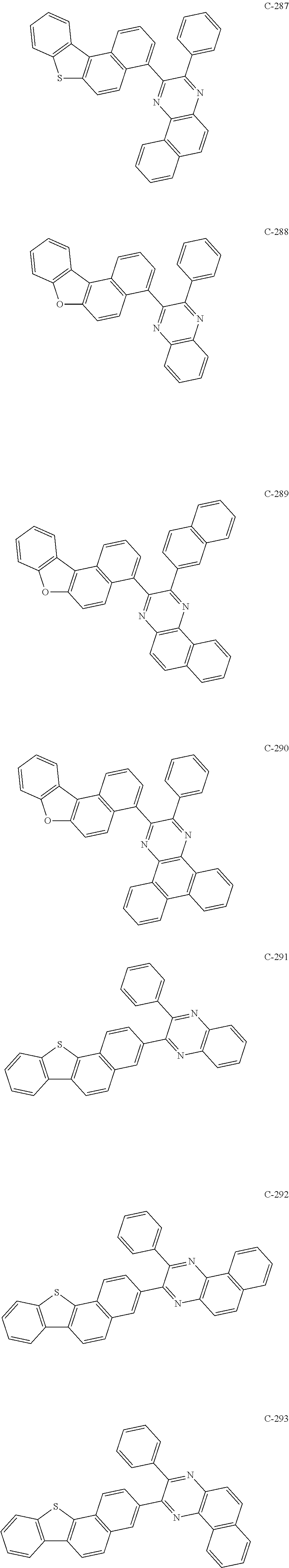

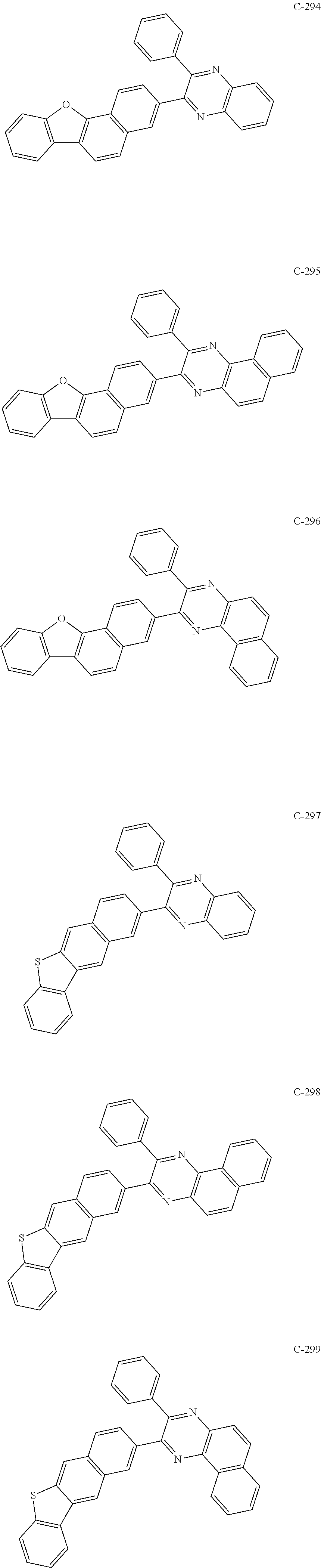 [SMILES: c1ccc(-c2nc3ccccc3nc2-c2ccc3c(ccc4c5ccccc5oc34)c2)cc1 ; c1ccc(-c2nc3c(ccc4ccccc43)nc2-c2ccc3c(ccc4c5ccccc5oc34)c2)cc1 ; c1ccc(-c2nc3ccc4ccccc4c3nc2-c2ccc3c(ccc4c5ccccc5oc34)c2)cc1 ; c1ccc(-c2nc3ccccc3nc2-c2ccc3cc4sc5ccccc5c4cc3c2)cc1 ; c1ccc(-c2nc3c(ccc4ccccc43)nc2-c2ccc3cc4sc5ccccc5c4cc3c2)cc1 ; c1ccc(-c2nc3ccc4ccccc4c3nc2-c2ccc3cc4sc5ccccc5c4cc3c2)cc1]

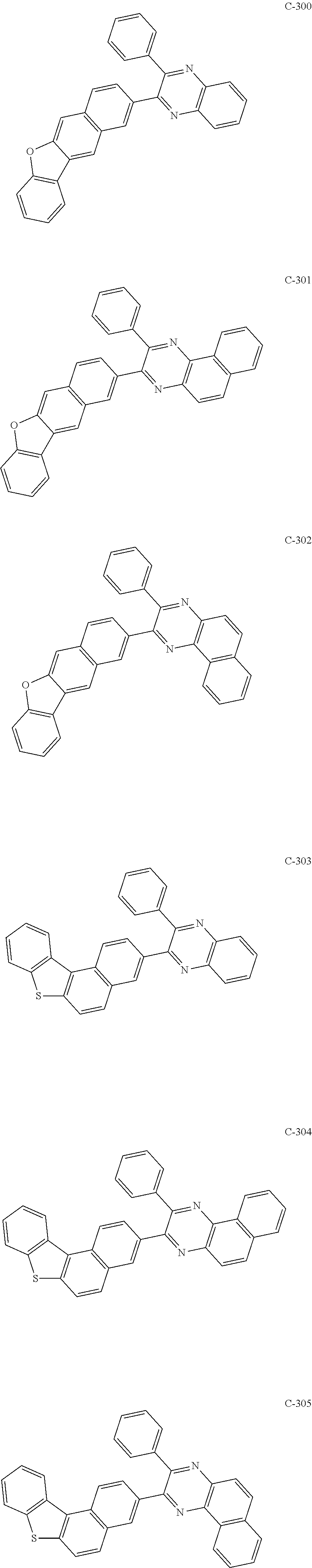 [SMILES: c1ccc(-c2nc3ccccc3nc2-c2ccc3cc4oc5ccccc5c4cc3c2)cc1 ; c1ccc(-c2nc3c(ccc4ccccc43)nc2-c2ccc3cc4oc5ccccc5c4cc3c2)cc1 ; c1ccc(-c2nc3ccc4ccccc4c3nc2-c2ccc3cc4oc5ccccc5c4cc3c2)cc1 ; c1ccc(-c2nc3ccccc3nc2-c2ccc3c(ccc4sc5ccccc5c43)c2)cc1 ; c1ccc(-c2nc3c(ccc4ccccc43)nc2-c2ccc3c(ccc4sc5ccccc5c43)c2)cc1 ; c1ccc(-c2nc3ccc4ccccc4c3nc2-c2ccc3c(ccc4sc5ccccc5c43)c2)cc1]

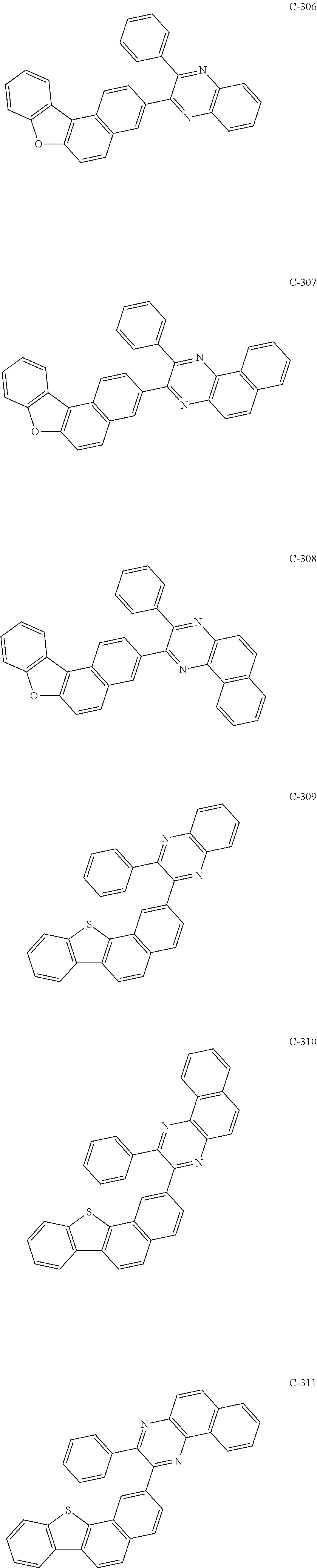 [SMILES: c1ccc(-c2nc3ccccc3nc2-c2ccc3c(ccc4oc5ccccc5c43)c2)cc1 ; c1ccc(-c2nc3c(ccc4ccccc43)nc2-c2ccc3c(ccc4oc5ccccc5c43)c2)cc1 ; c1ccc(-c2nc3ccc4ccccc4c3nc2-c2ccc3c(ccc4oc5ccccc5c43)c2)cc1 ; c1ccc(-c2nc3ccccc3nc2-c2ccc3ccc4c5ccccc5sc4c3c2)cc1 ; c1ccc(-c2nc3c(ccc4ccccc43)nc2-c2ccc3ccc4c5ccccc5sc4c3c2)cc1 ; c1ccc(-c2nc3ccc4ccccc4c3nc2-c2ccc3ccc4c5ccccc5sc4c3c2)cc1]

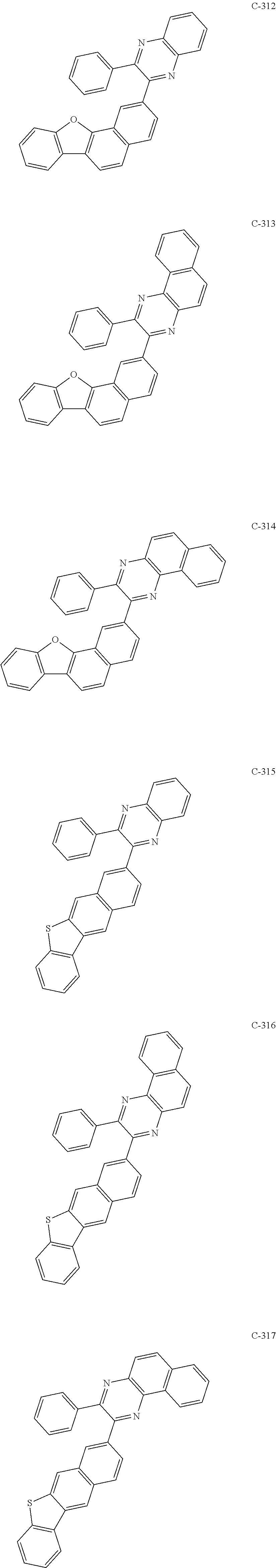 [SMILES: c1ccc(-c2nc3ccccc3nc2-c2ccc3ccc4c5ccccc5oc4c3c2)cc1 ; c1ccc(-c2nc3c(ccc4ccccc43)nc2-c2ccc3ccc4c5ccccc5oc4c3c2)cc1 ; c1ccc(-c2nc3ccc4ccccc4c3nc2-c2ccc3ccc4c5ccccc5oc4c3c2)cc1 ; c1ccc(-c2nc3ccccc3nc2-c2ccc3cc4c(cc3c2)sc2ccccc24)cc1 ; c1ccc(-c2nc3c(ccc4ccccc43)nc2-c2ccc3cc4c(cc3c2)sc2ccccc24)cc1 ; c1ccc(-c2nc3ccc4ccccc4c3nc2-c2ccc3cc4c(cc3c2)sc2ccccc24)cc1]

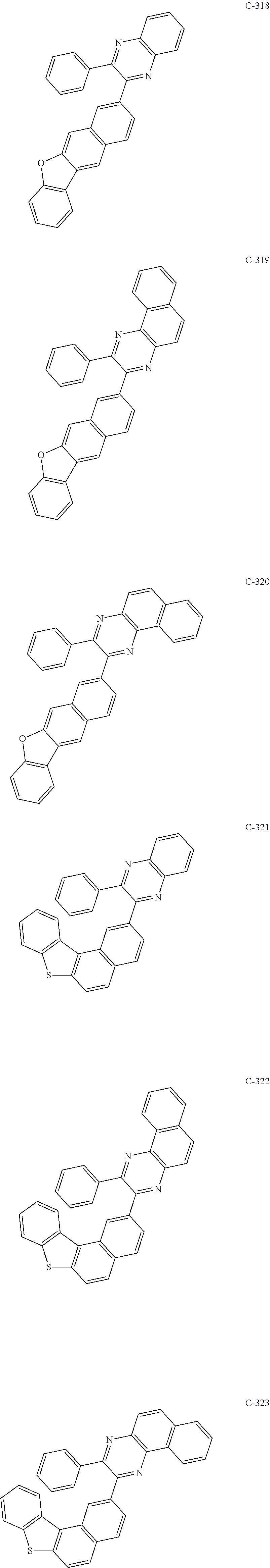 [SMILES: c1ccc(-c2nc3ccccc3nc2-c2ccc3cc4c(cc3c2)oc2ccccc24)cc1 ; c1ccc(-c2nc3c(ccc4ccccc43)nc2-c2ccc3cc4c(cc3c2)oc2ccccc24)cc1 ; c1ccc(-c2nc3ccc4ccccc4c3nc2-c2ccc3cc4c(cc3c2)oc2ccccc24)cc1 ; c1ccc(-c2nc3ccccc3nc2-c2ccc3ccc4sc5ccccc5c4c3c2)cc1 ; c1ccc(-c2nc3c(ccc4ccccc43)nc2-c2ccc3ccc4sc5ccccc5c4c3c2)cc1 ; c1ccc(-c2nc3ccc4ccccc4c3nc2-c2ccc3ccc4sc5ccccc5c4c3c2)cc1]

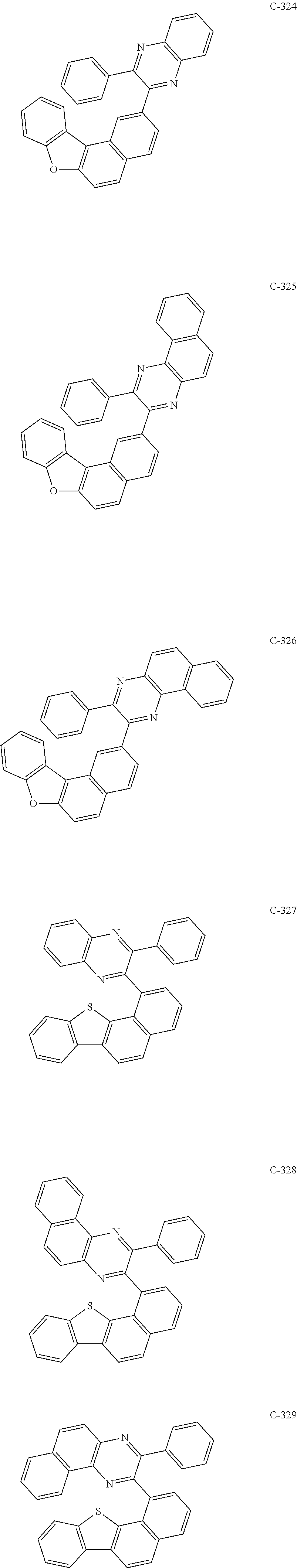 [SMILES: c1ccc(-c2nc3ccccc3nc2-c2ccc3ccc4oc5ccccc5c4c3c2)cc1 ; c1ccc(-c2nc3c(ccc4ccccc43)nc2-c2ccc3ccc4oc5ccccc5c4c3c2)cc1 ; c1ccc(-c2nc3ccc4ccccc4c3nc2-c2ccc3ccc4oc5ccccc5c4c3c2)cc1 ; c1ccc(-c2nc3ccccc3nc2-c2cccc3ccc4c5ccccc5sc4c23)cc1 ; c1ccc(-c2nc3c(ccc4ccccc43)nc2-c2cccc3ccc4c5ccccc5sc4c23)cc1 ; c1ccc(-c2nc3ccc4ccccc4c3nc2-c2cccc3ccc4c5ccccc5sc4c23)cc1]

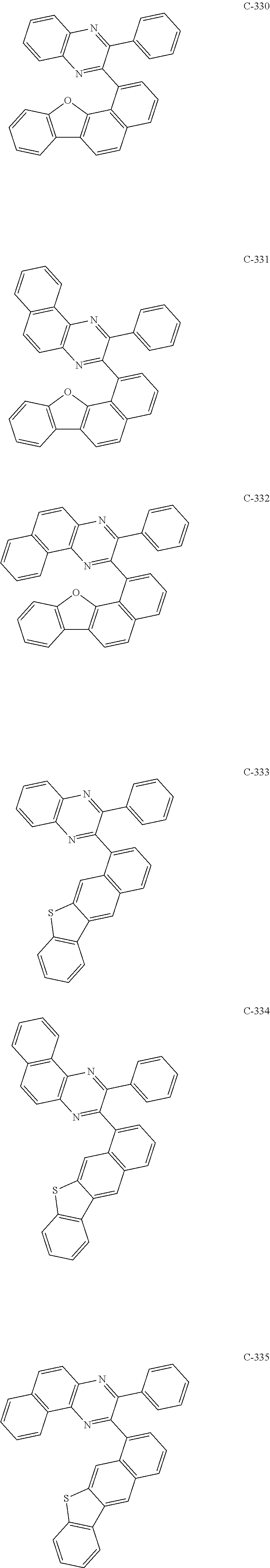 [SMILES: c1ccc(-c2nc3ccccc3nc2-c2cccc3ccc4c5ccccc5oc4c23)cc1 ; c1ccc(-c2nc3c(ccc4ccccc43)nc2-c2cccc3ccc4c5ccccc5oc4c23)cc1 ; c1ccc(-c2nc3ccc4ccccc4c3nc2-c2cccc3ccc4c5ccccc5oc4c23)cc1 ; c1ccc(-c2nc3ccccc3nc2-c2cccc3cc4c(cc23)sc2ccccc24)cc1 ; c1ccc(-c2nc3c(ccc4ccccc43)nc2-c2cccc3cc4c(cc23)sc2ccccc24)cc1 ; c1ccc(-c2nc3ccc4ccccc4c3nc2-c2cccc3cc4c(cc23)sc2ccccc24)cc1]

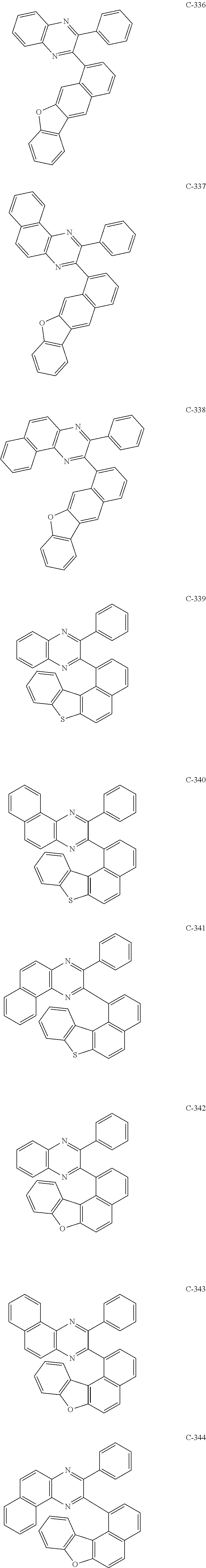

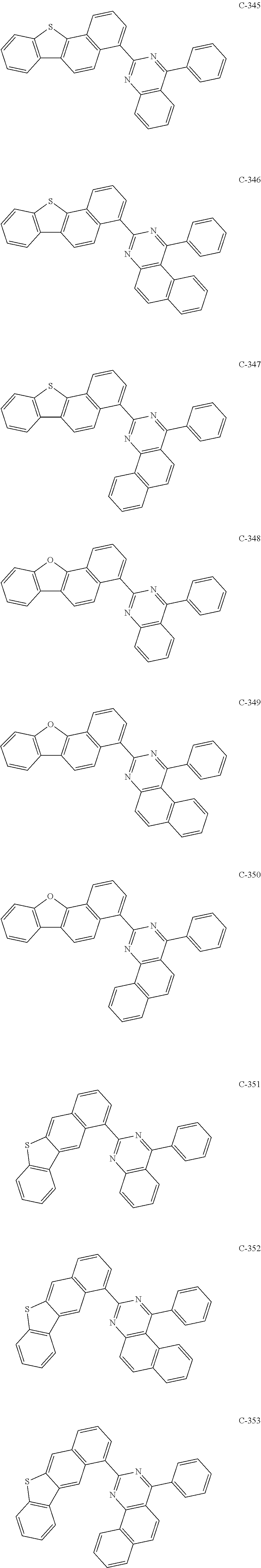

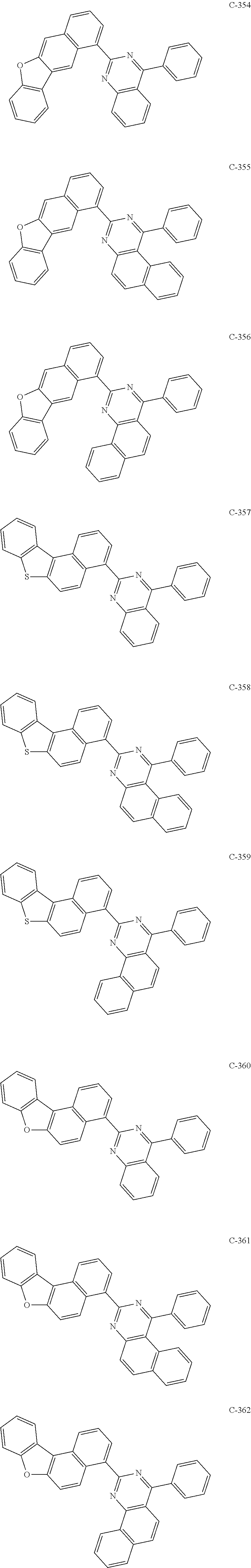

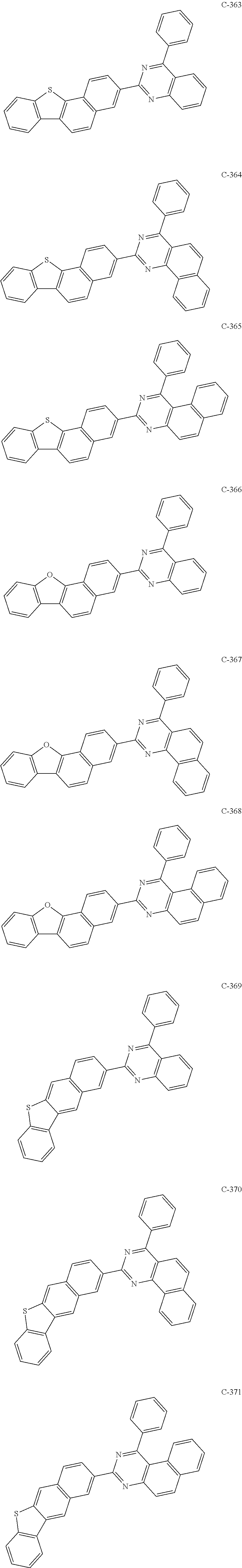

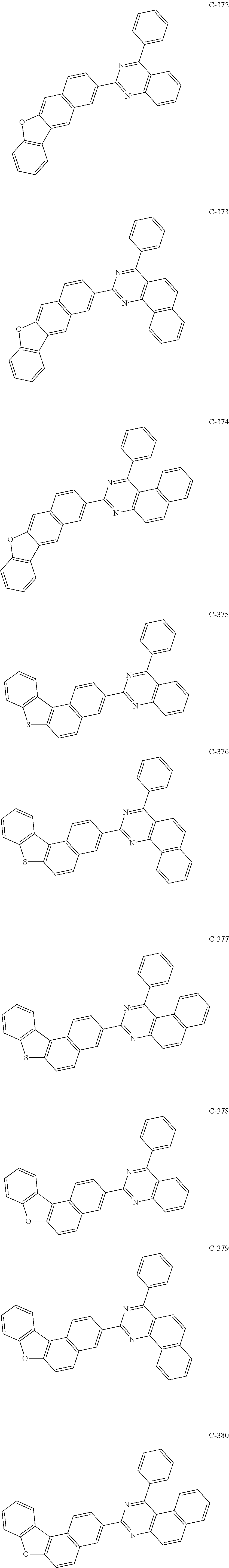

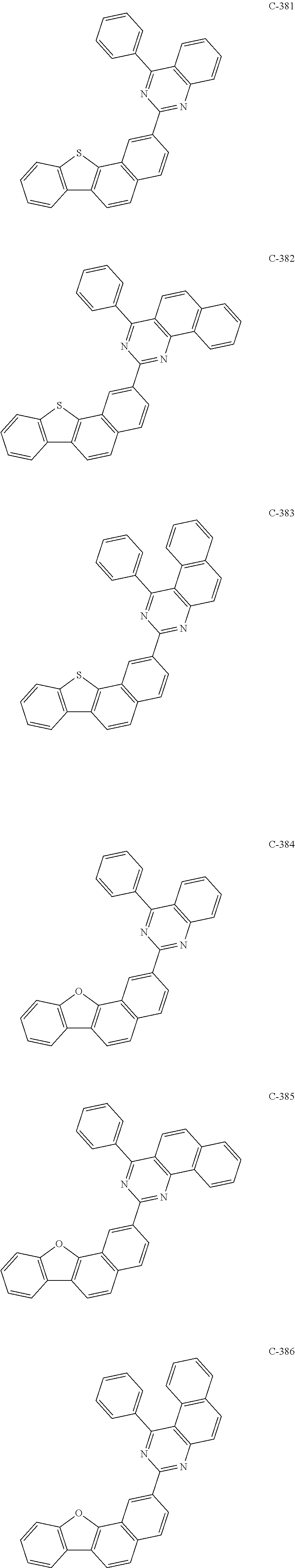 [SMILES: c1ccc(-c2nc(-c3ccc4ccc5c6ccccc6sc5c4c3)nc3ccccc23)cc1 ; c1ccc(-c2nc(-c3ccc4ccc5c6ccccc6sc5c4c3)nc3c2ccc2ccccc23)cc1 ; c1ccc(-c2nc(-c3ccc4ccc5c6ccccc6sc5c4c3)nc3ccc4ccccc4c23)cc1 ; c1ccc(-c2nc(-c3ccc4ccc5c6ccccc6oc5c4c3)nc3ccccc23)cc1 ; c1ccc(-c2nc(-c3ccc4ccc5c6ccccc6oc5c4c3)nc3c2ccc2ccccc23)cc1 ; c1ccc(-c2nc(-c3ccc4ccc5c6ccccc6oc5c4c3)nc3ccc4ccccc4c23)cc1]

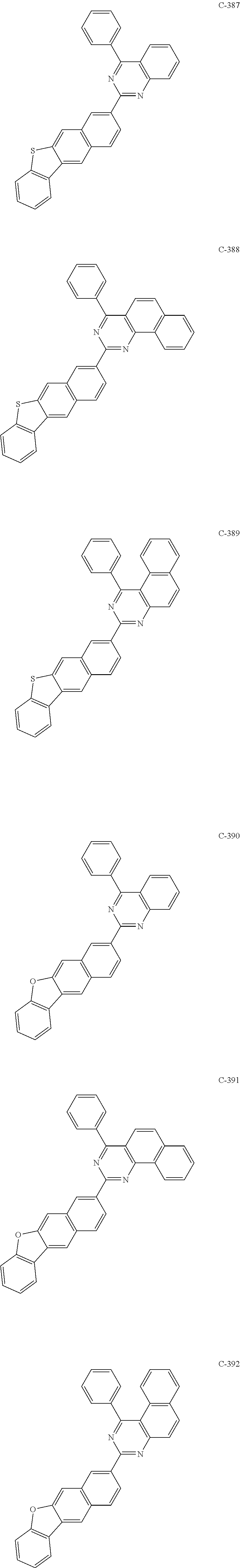 [SMILES: c1ccc(-c2nc(-c3ccc4cc5c(cc4c3)sc3ccccc35)nc3ccccc23)cc1 ; c1ccc(-c2nc(-c3ccc4cc5c(cc4c3)sc3ccccc35)nc3c2ccc2ccccc23)cc1 ; c1ccc(-c2nc(-c3ccc4cc5c(cc4c3)sc3ccccc35)nc3ccc4ccccc4c23)cc1 ; c1ccc(-c2nc(-c3ccc4cc5c(cc4c3)oc3ccccc35)nc3ccccc23)cc1 ; c1ccc(-c2nc(-c3ccc4cc5c(cc4c3)oc3ccccc35)nc3c2ccc2ccccc23)cc1 ; c1ccc(-c2nc(-c3ccc4cc5c(cc4c3)oc3ccccc35)nc3ccc4ccccc4c23)cc1]

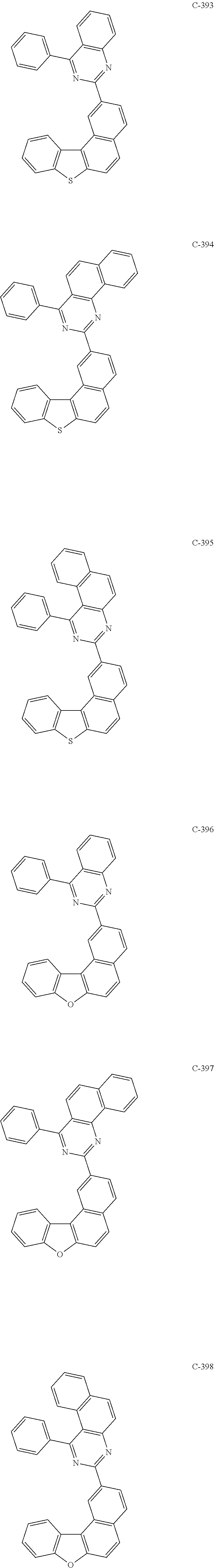 [SMILES: c1ccc(-c2nc(-c3ccc4ccc5sc6ccccc6c5c4c3)nc3ccccc23)cc1 ; c1ccc(-c2nc(-c3ccc4ccc5sc6ccccc6c5c4c3)nc3c2ccc2ccccc23)cc1 ; c1ccc(-c2nc(-c3ccc4ccc5sc6ccccc6c5c4c3)nc3ccc4ccccc4c23)cc1 ; c1ccc(-c2nc(-c3ccc4ccc5oc6ccccc6c5c4c3)nc3ccccc23)cc1 ; c1ccc(-c2nc(-c3ccc4ccc5oc6ccccc6c5c4c3)nc3c2ccc2ccccc23)cc1 ; c1ccc(-c2nc(-c3ccc4ccc5oc6ccccc6c5c4c3)nc3ccc4ccccc4c23)cc1]

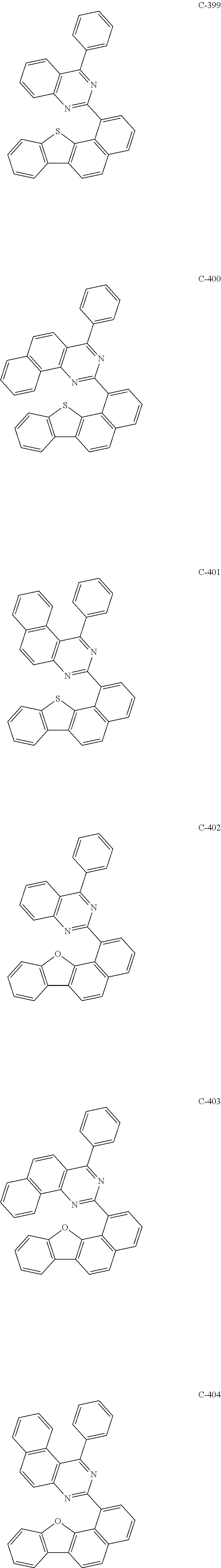 [SMILES: c1ccc(-c2nc(-c3cccc4ccc5c6ccccc6sc5c34)nc3ccccc23)cc1 ; c1ccc(-c2nc(-c3cccc4ccc5c6ccccc6sc5c34)nc3c2ccc2ccccc23)cc1 ; c1ccc(-c2nc(-c3cccc4ccc5c6ccccc6sc5c34)nc3ccc4ccccc4c23)cc1 ; c1ccc(-c2nc(-c3cccc4ccc5c6ccccc6oc5c34)nc3ccccc23)cc1 ; c1ccc(-c2nc(-c3cccc4ccc5c6ccccc6oc5c34)nc3c2ccc2ccccc23)cc1 ; c1ccc(-c2nc(-c3cccc4ccc5c6ccccc6oc5c34)nc3ccc4ccccc4c23)cc1]

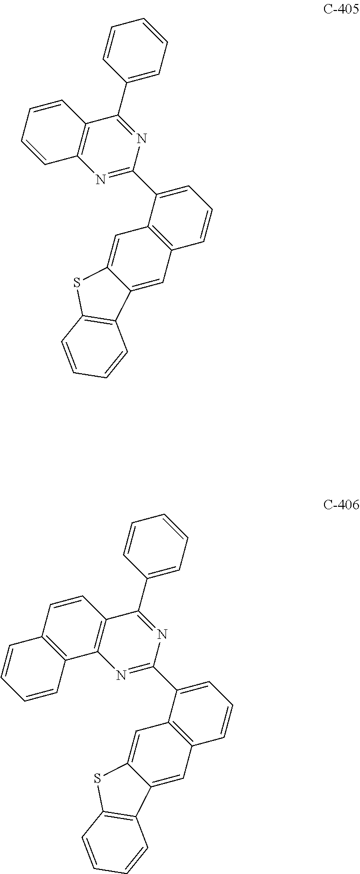 [SMILES: c1ccc(-c2nc(-c3cccc4cc5c(cc34)sc3ccccc35)nc3ccccc23)cc1 ; c1ccc(-c2nc(-c3cccc4cc5c(cc34)sc3ccccc35)nc3c2ccc2ccccc23)cc1]

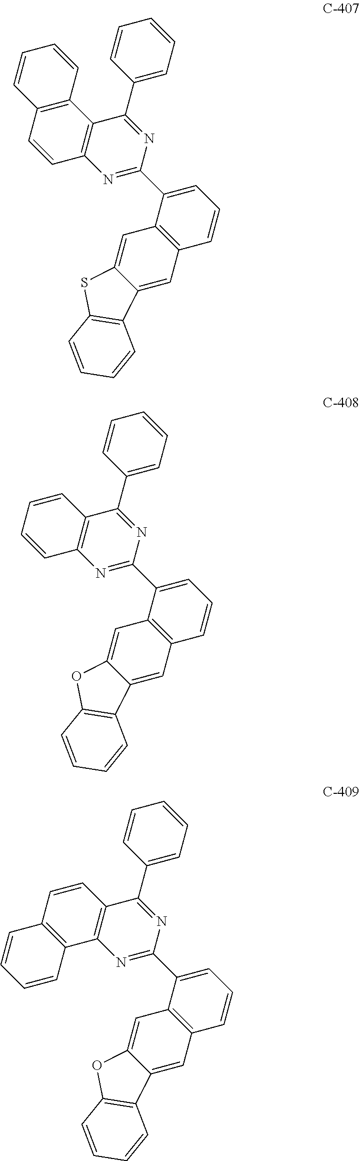

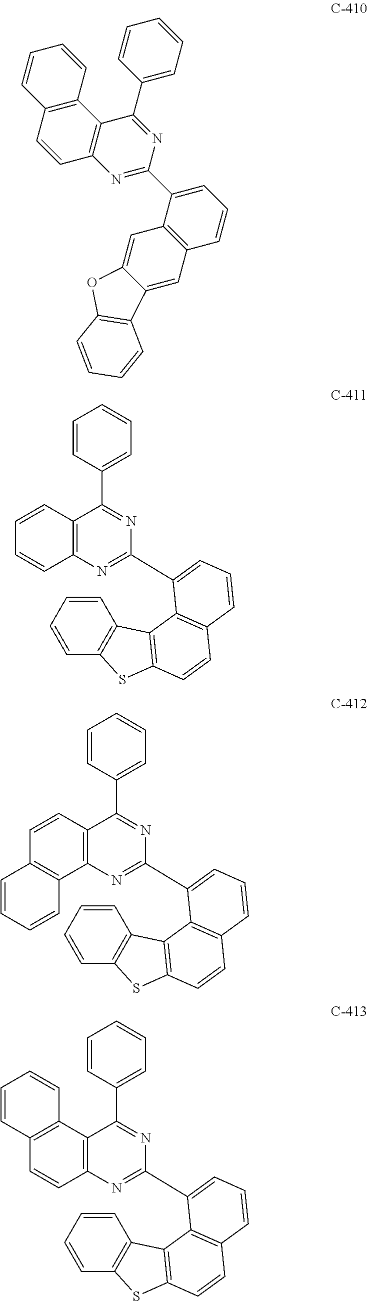

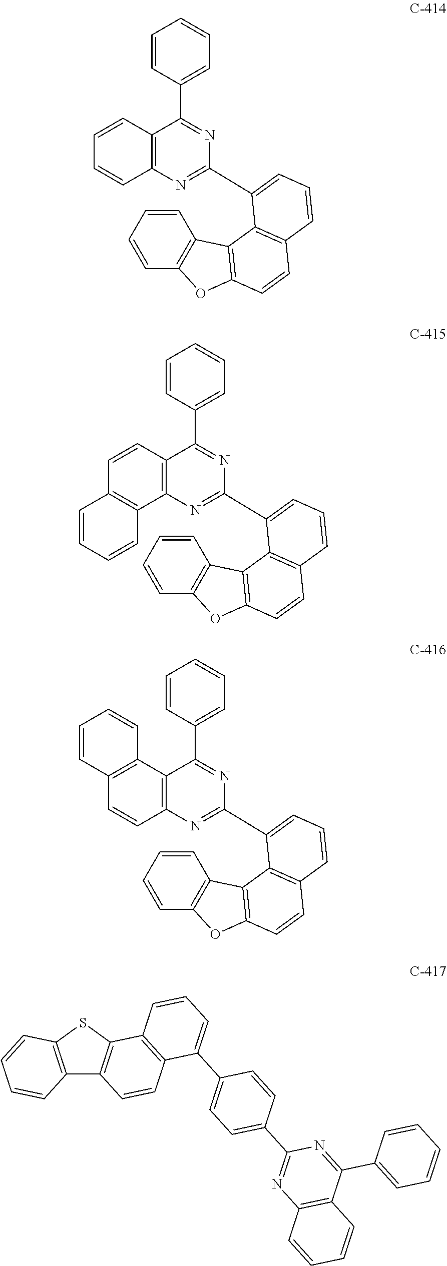

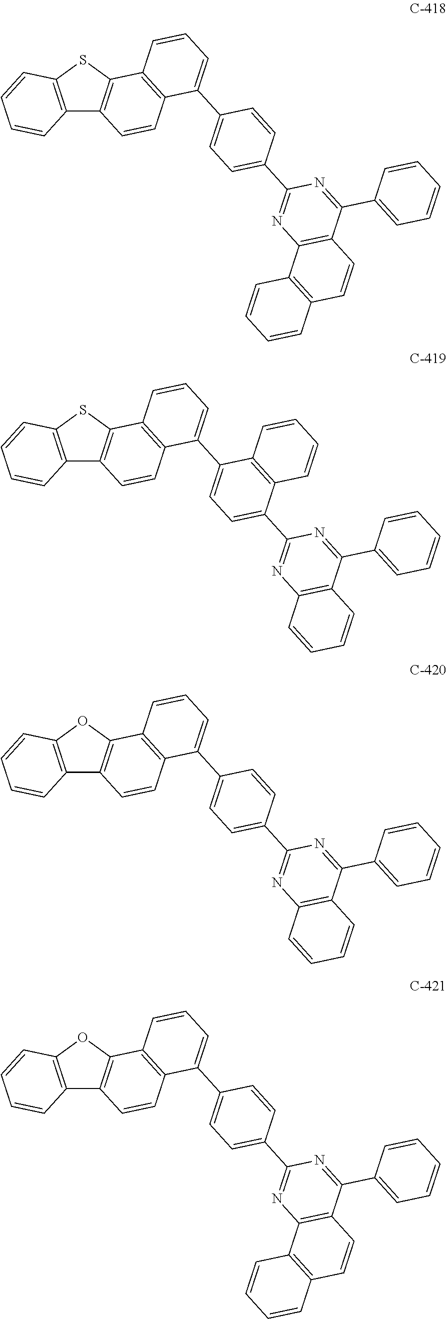 [SMILES: c1ccc(-c2nc(-c3ccc(-c4cccc5c4ccc4c6ccccc6sc54)cc3)nc3c2ccc2ccccc23)cc1 ; c1ccc(-c2nc(-c3ccc(-c4cccc5c4ccc4c6ccccc6sc54)c4ccccc34)nc3ccccc23)cc1 ; c1ccc(-c2nc(-c3ccc(-c4cccc5c4ccc4c6ccccc6oc54)cc3)nc3ccccc23)cc1 ; c1ccc(-c2nc(-c3ccc(-c4cccc5c4ccc4c6ccccc6oc54)cc3)nc3c2ccc2ccccc23)cc1]

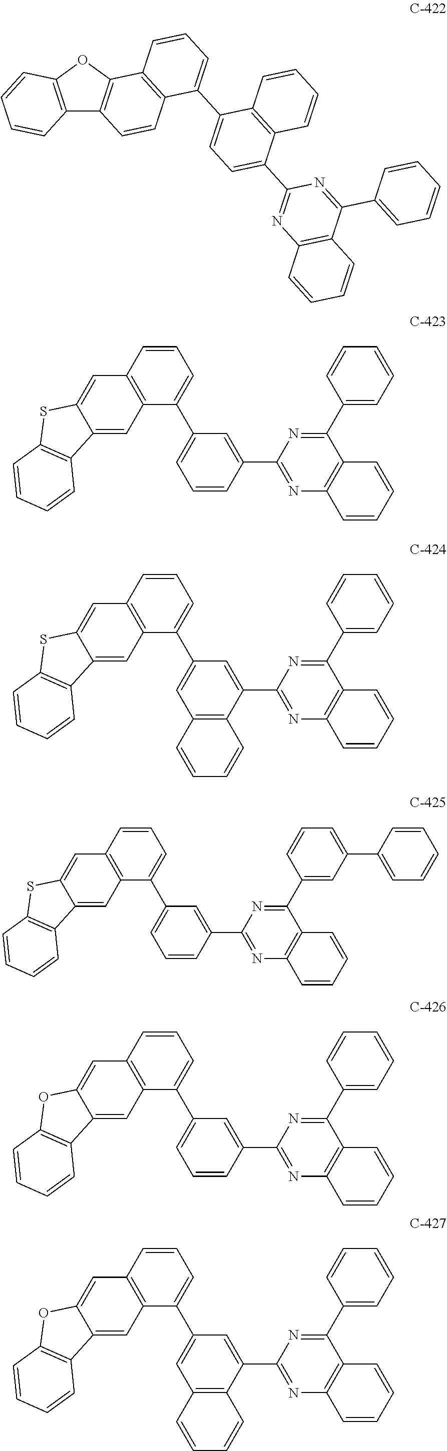

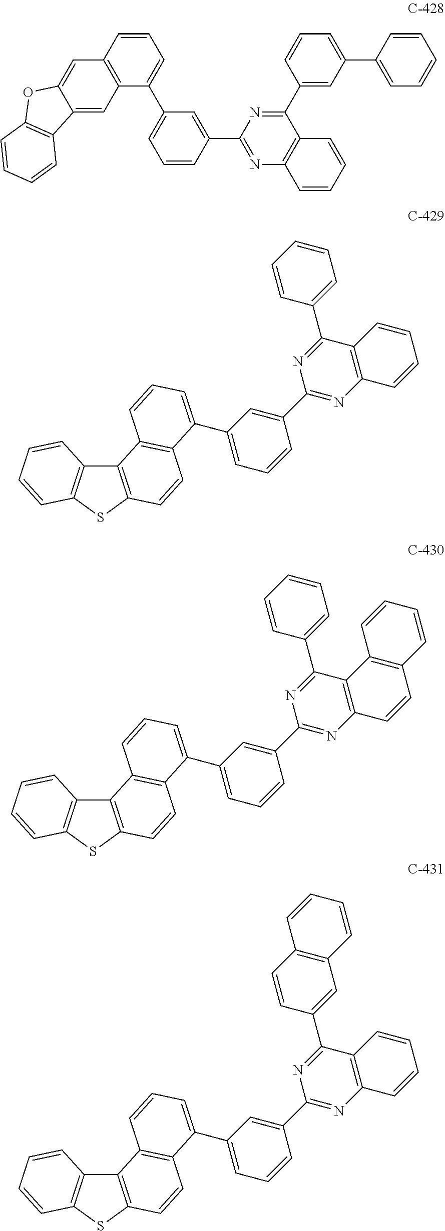 [SMILES: c1ccc(-c2cccc(-c3nc(-c4cccc(-c5cccc6cc7oc8ccccc8c7cc56)c4)nc4ccccc34)c2)cc1 ; c1ccc(-c2nc(-c3cccc(-c4cccc5c4ccc4sc6ccccc6c45)c3)nc3ccccc23)cc1 ; c1ccc(-c2nc(-c3cccc(-c4cccc5c4ccc4sc6ccccc6c45)c3)nc3ccc4ccccc4c23)cc1 ; c1cc(-c2nc(-c3ccc4ccccc4c3)c3ccccc3n2)cc(-c2cccc3c2ccc2sc4ccccc4c23)c1]

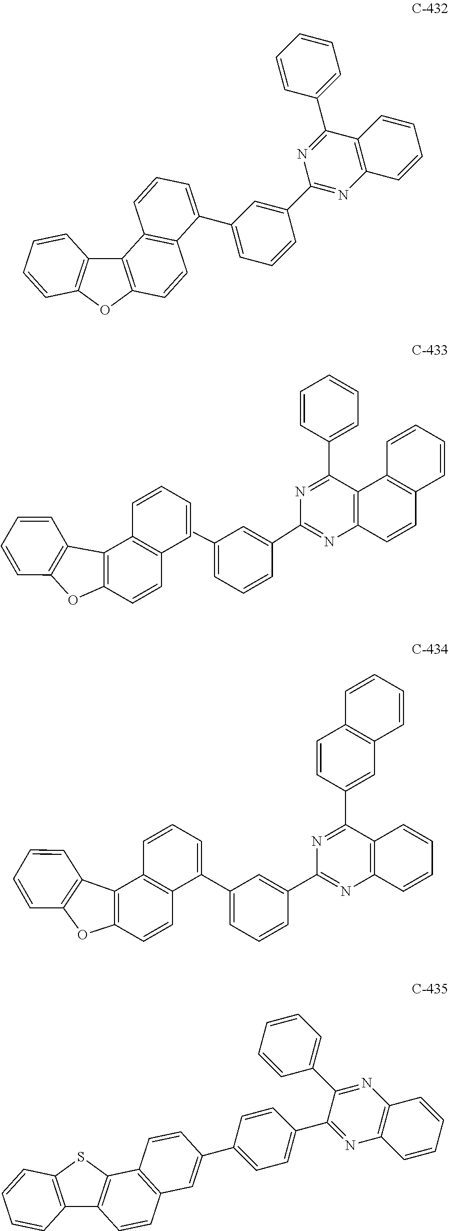

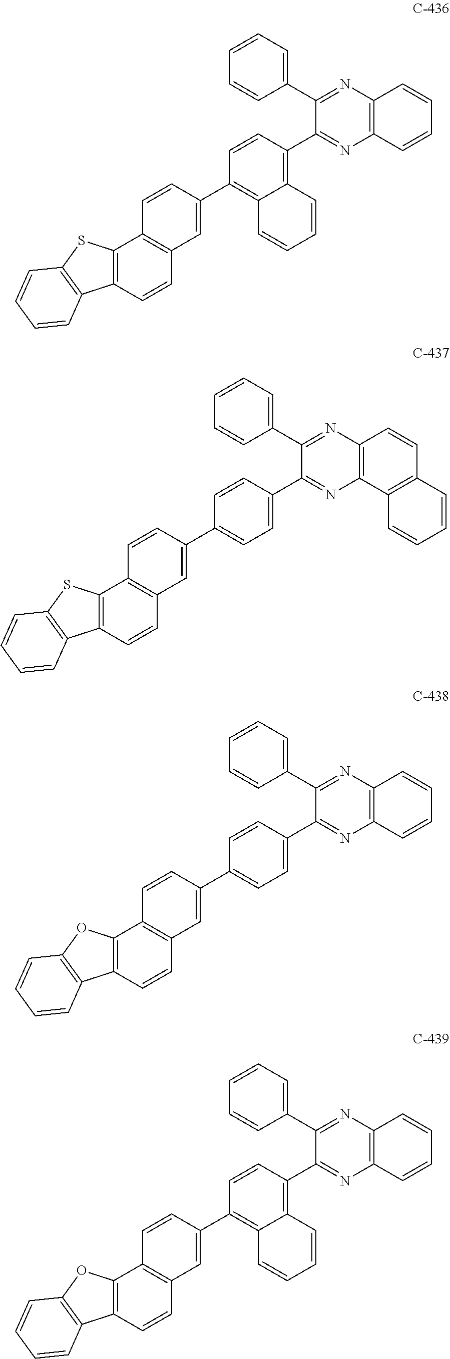 [SMILES: c1ccc(-c2nc3ccccc3nc2-c2ccc(-c3ccc4c(ccc5c6ccccc6sc45)c3)c3ccccc23)cc1 ; c1ccc(-c2nc3ccc4ccccc4c3nc2-c2ccc(-c3ccc4c(ccc5c6ccccc6sc45)c3)cc2)cc1 ; c1ccc(-c2nc3ccccc3nc2-c2ccc(-c3ccc4c(ccc5c6ccccc6oc45)c3)cc2)cc1 ; c1ccc(-c2nc3ccccc3nc2-c2ccc(-c3ccc4c(ccc5c6ccccc6oc45)c3)c3ccccc23)cc1]

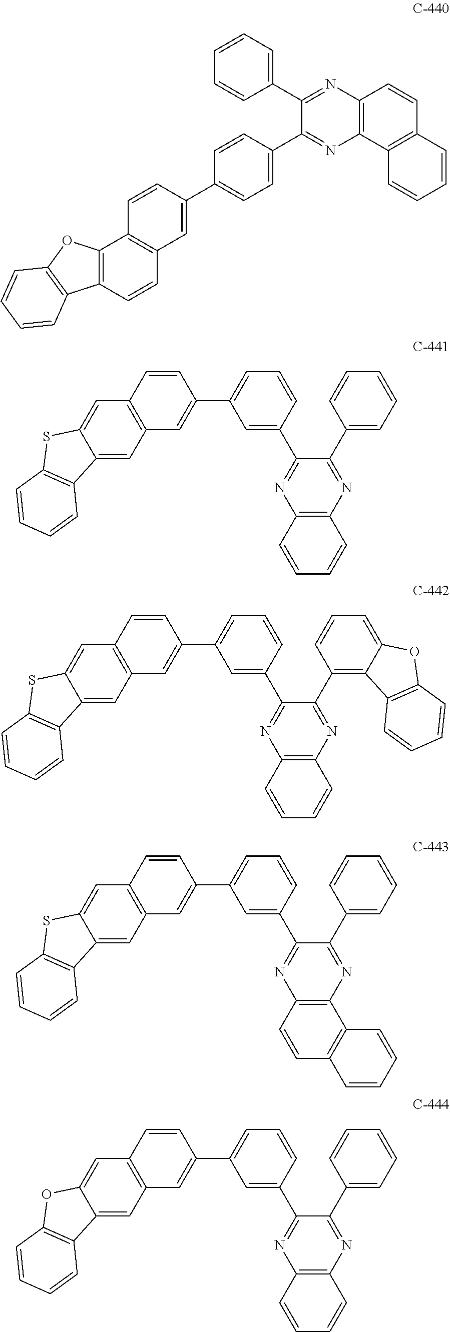

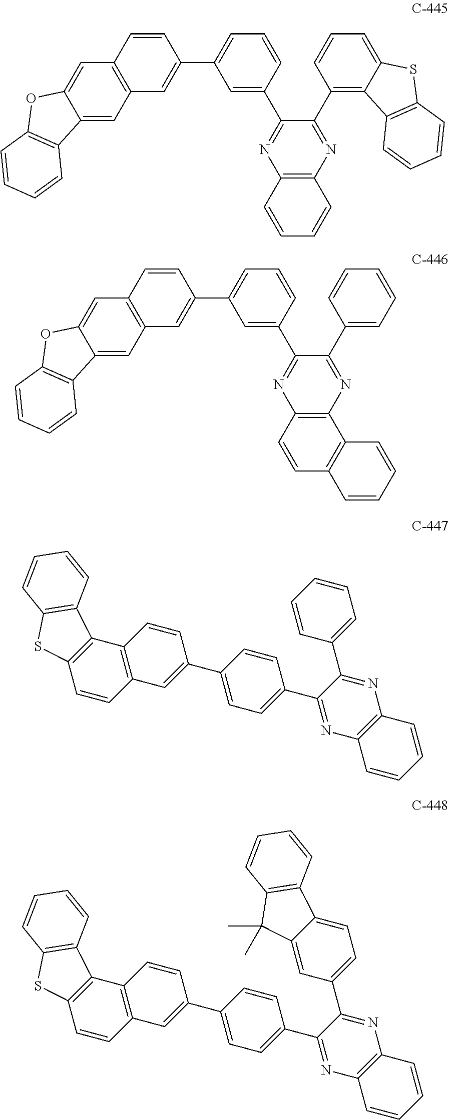

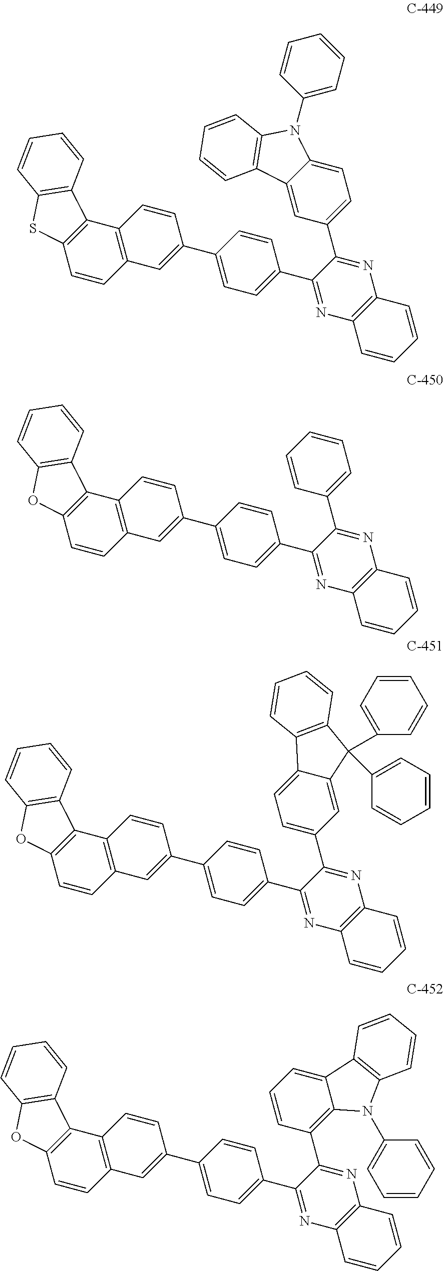

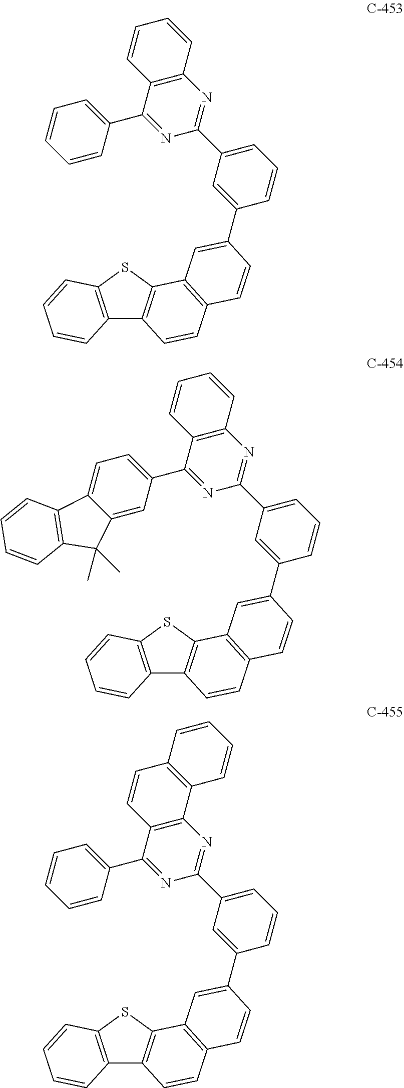

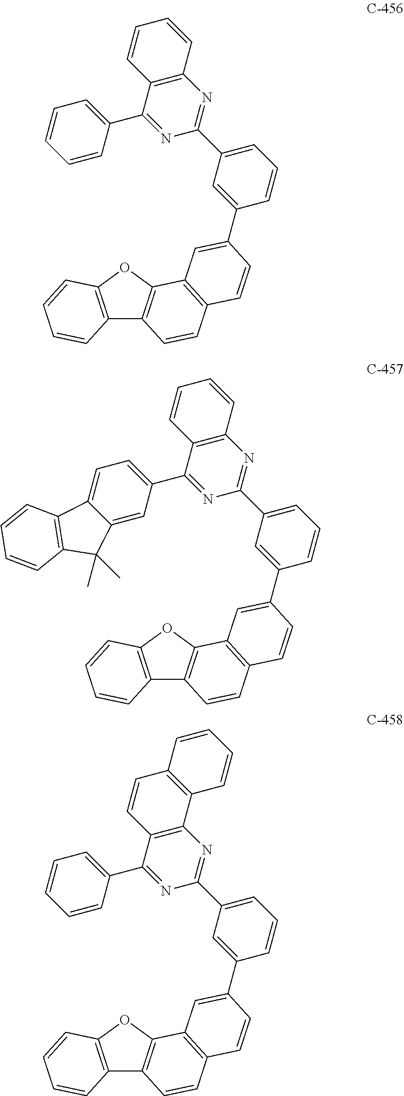

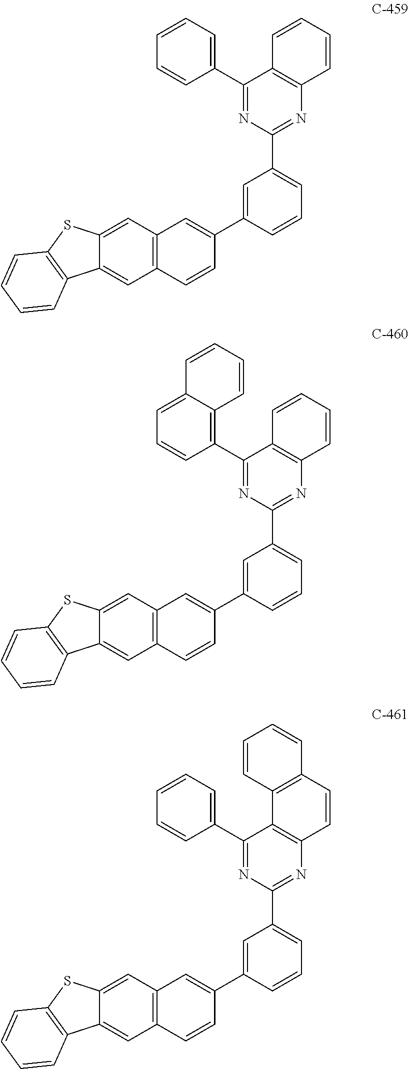 [SMILES: c1ccc(-c2nc(-c3cccc(-c4ccc5cc6c(cc5c4)sc4ccccc46)c3)nc3ccccc23)cc1 ; c1cc(-c2ccc3cc4c(cc3c2)sc2ccccc24)cc(-c2nc(-c3cccc4ccccc34)c3ccccc3n2)c1 ; c1ccc(-c2nc(-c3cccc(-c4ccc5cc6c(cc5c4)sc4ccccc46)c3)nc3ccc4ccccc4c23)cc1]

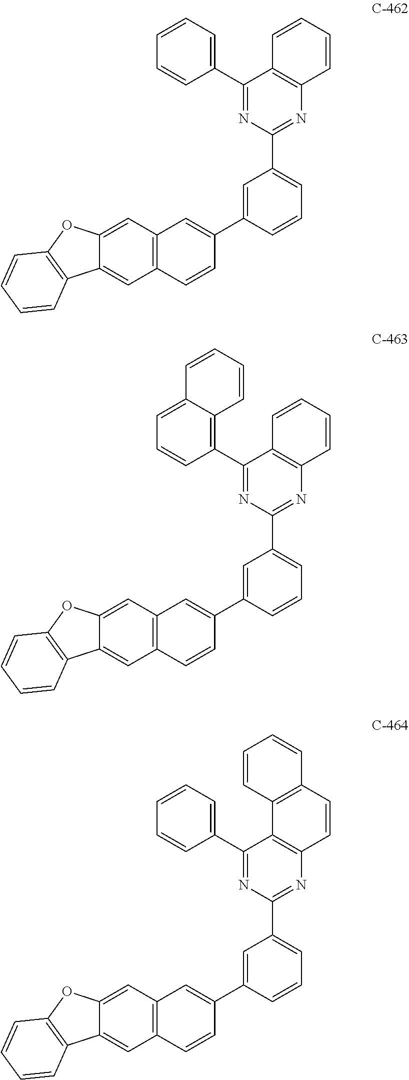 [SMILES: c1ccc(-c2nc(-c3cccc(-c4ccc5cc6c(cc5c4)oc4ccccc46)c3)nc3ccccc23)cc1 ; c1cc(-c2ccc3cc4c(cc3c2)oc2ccccc24)cc(-c2nc(-c3cccc4ccccc34)c3ccccc3n2)c1 ; c1ccc(-c2nc(-c3cccc(-c4ccc5cc6c(cc5c4)oc4ccccc46)c3)nc3ccc4ccccc4c23)cc1]

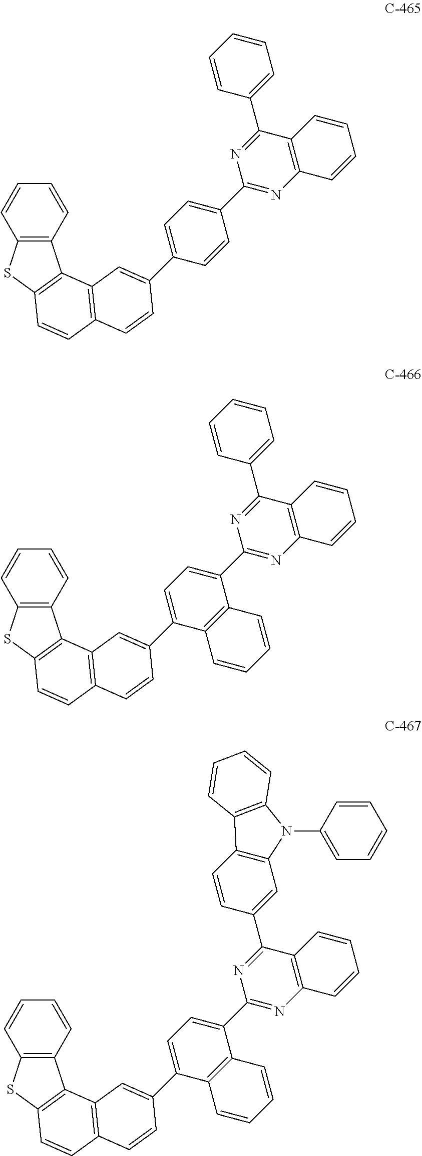 [SMILES: c1ccc(-c2nc(-c3ccc(-c4ccc5ccc6sc7ccccc7c6c5c4)cc3)nc3ccccc23)cc1 ; c1ccc(-c2nc(-c3ccc(-c4ccc5ccc6sc7ccccc7c6c5c4)c4ccccc34)nc3ccccc23)cc1 ; c1ccc(-n2c3ccccc3c3ccc(-c4nc(-c5ccc(-c6ccc7ccc8sc9ccccc9c8c7c6)c6ccccc56)nc5ccccc45)cc32)cc1]

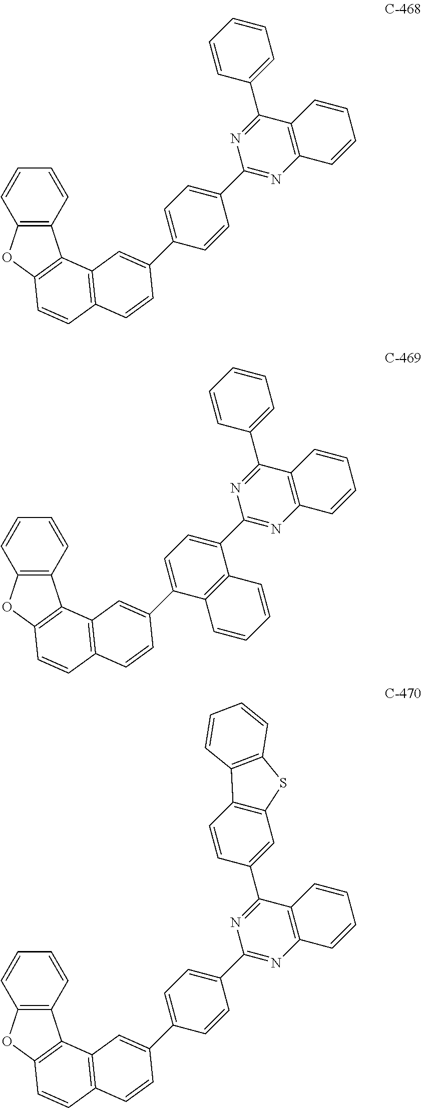 [SMILES: c1ccc(-c2nc(-c3ccc(-c4ccc5ccc6oc7ccccc7c6c5c4)cc3)nc3ccccc23)cc1 ; c1ccc(-c2nc(-c3ccc(-c4ccc5ccc6oc7ccccc7c6c5c4)c4ccccc34)nc3ccccc23)cc1 ; c1ccc2c(-c3ccc4c(c3)sc3ccccc34)nc(-c3ccc(-c4ccc5ccc6oc7ccccc7c6c5c4)cc3)nc2c1]

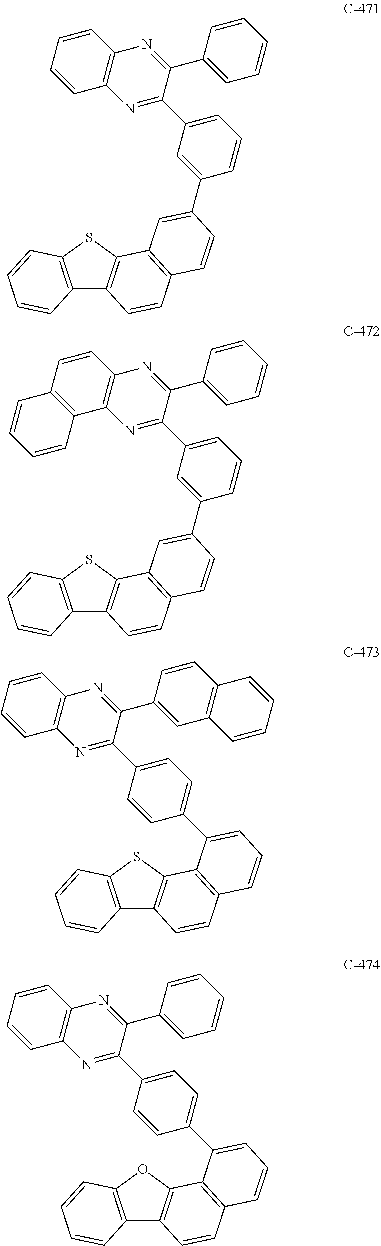

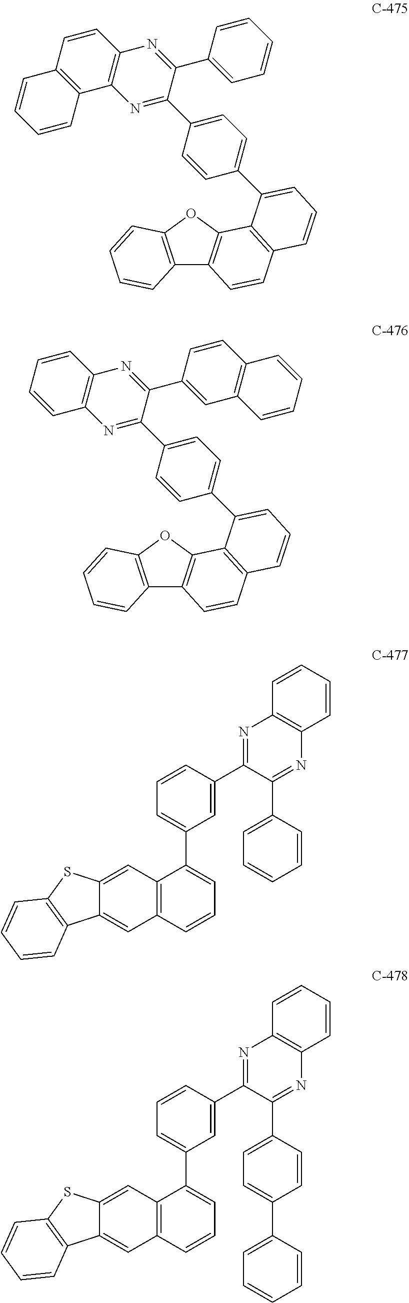 [SMILES: c1ccc(-c2nc3ccc4ccccc4c3nc2-c2ccc(-c3cccc4ccc5c6ccccc6oc5c34)cc2)cc1 ; c1ccc2cc(-c3nc4ccccc4nc3-c3ccc(-c4cccc5ccc6c7ccccc7oc6c45)cc3)ccc2c1 ; c1ccc(-c2nc3ccccc3nc2-c2cccc(-c3cccc4cc5c(cc34)sc3ccccc35)c2)cc1 ; c1ccc(-c2ccc(-c3nc4ccccc4nc3-c3cccc(-c4cccc5cc6c(cc45)sc4ccccc46)c3)cc2)cc1]

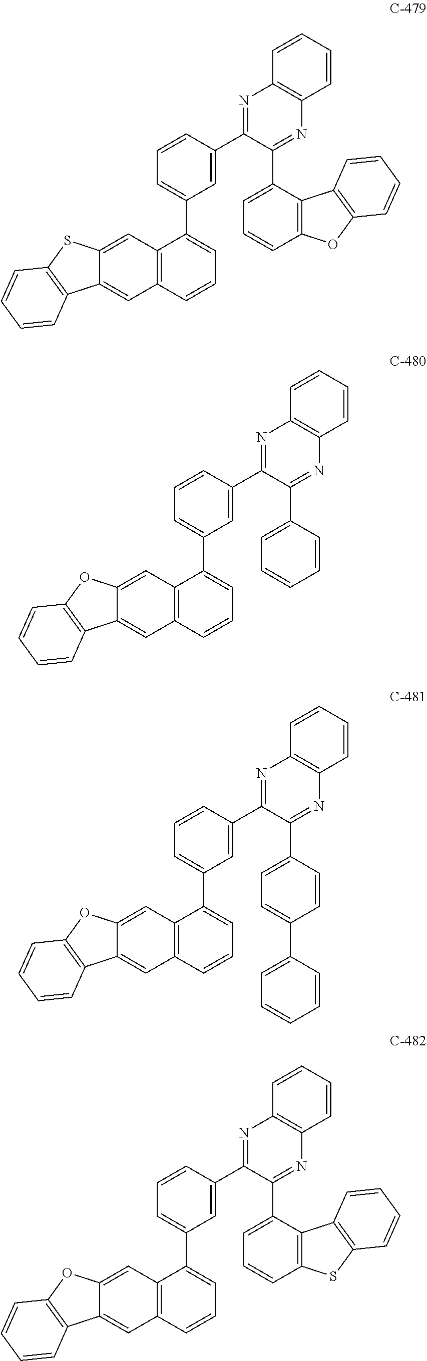

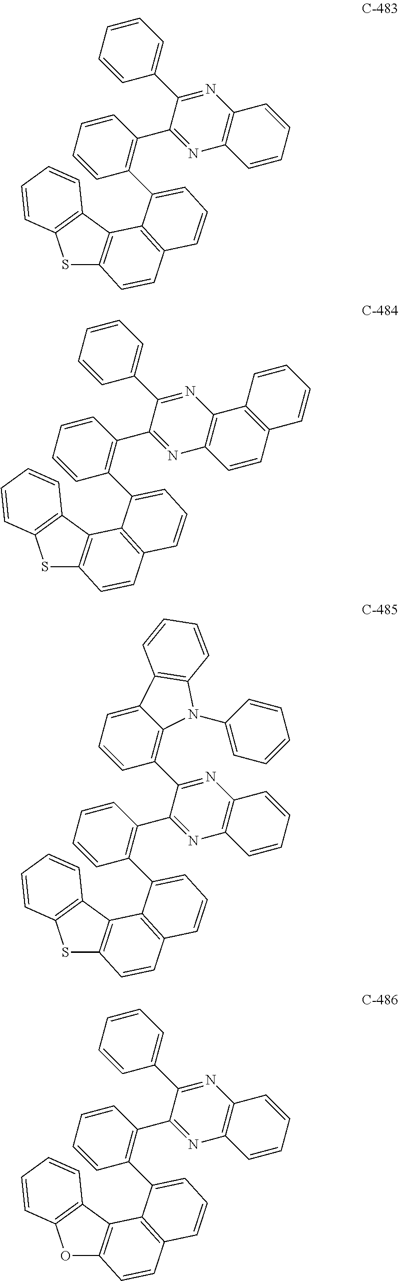

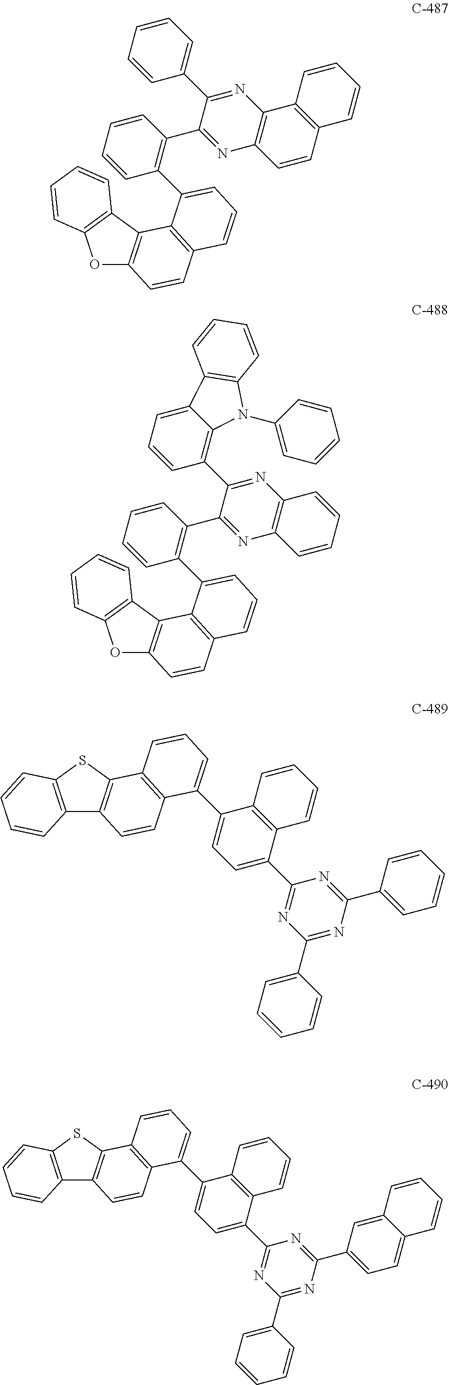 [SMILES: c1ccc(-c2nc3c(ccc4ccccc43)nc2-c2ccccc2-c2cccc3ccc4oc5ccccc5c4c23)cc1 ; c1ccc(-n2c3ccccc3c3cccc(-c4nc5ccccc5nc4-c4ccccc4-c4cccc5ccc6oc7ccccc7c6c45)c32)cc1 ; c1ccc(-c2nc(-c3ccccc3)nc(-c3ccc(-c4cccc5c4ccc4c6ccccc6sc54)c4ccccc34)n2)cc1 ; c1ccc(-c2nc(-c3ccc4ccccc4c3)nc(-c3ccc(-c4cccc5c4ccc4c6ccccc6sc54)c4ccccc34)n2)cc1]

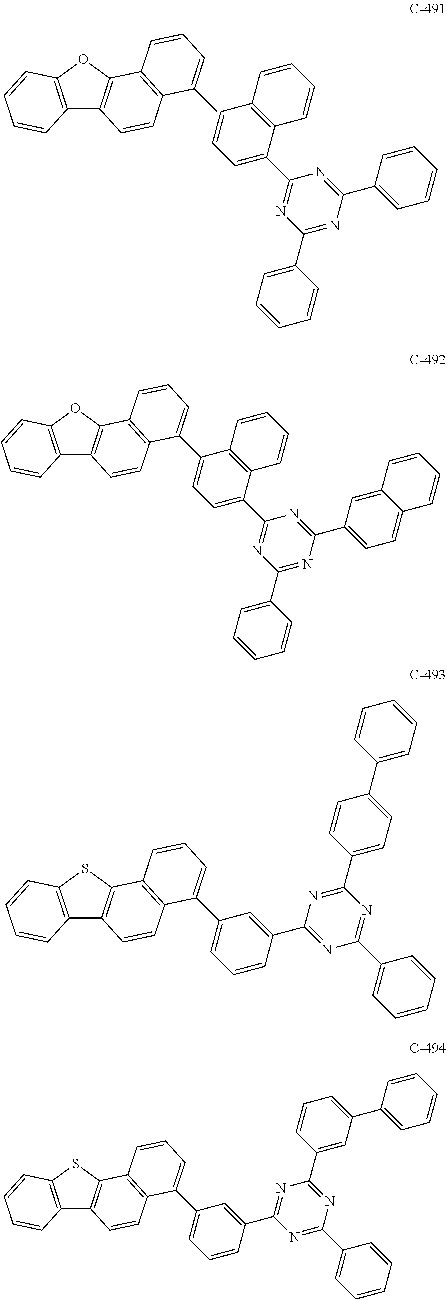

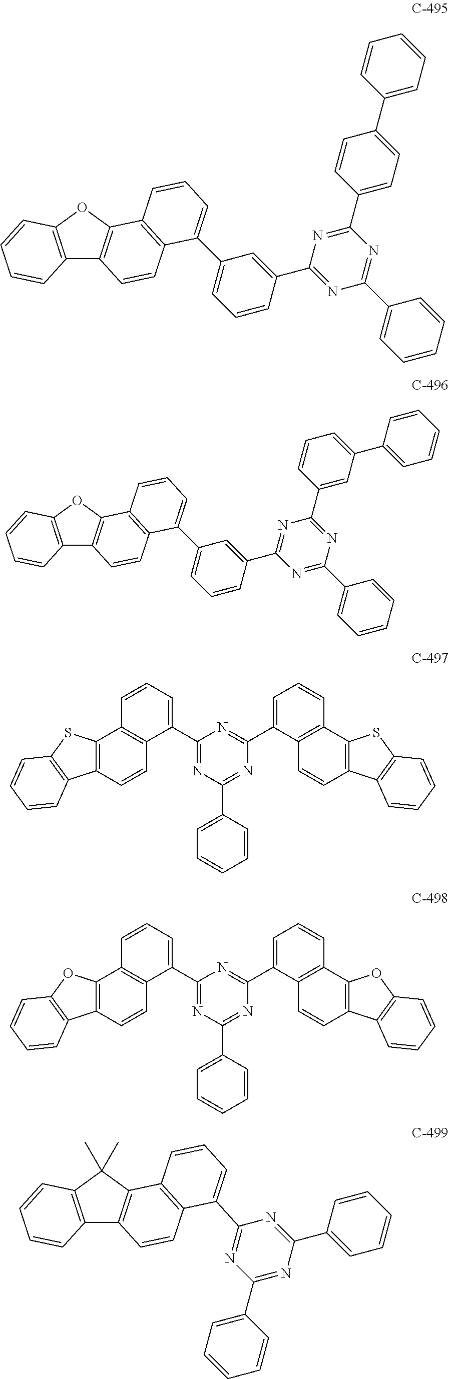

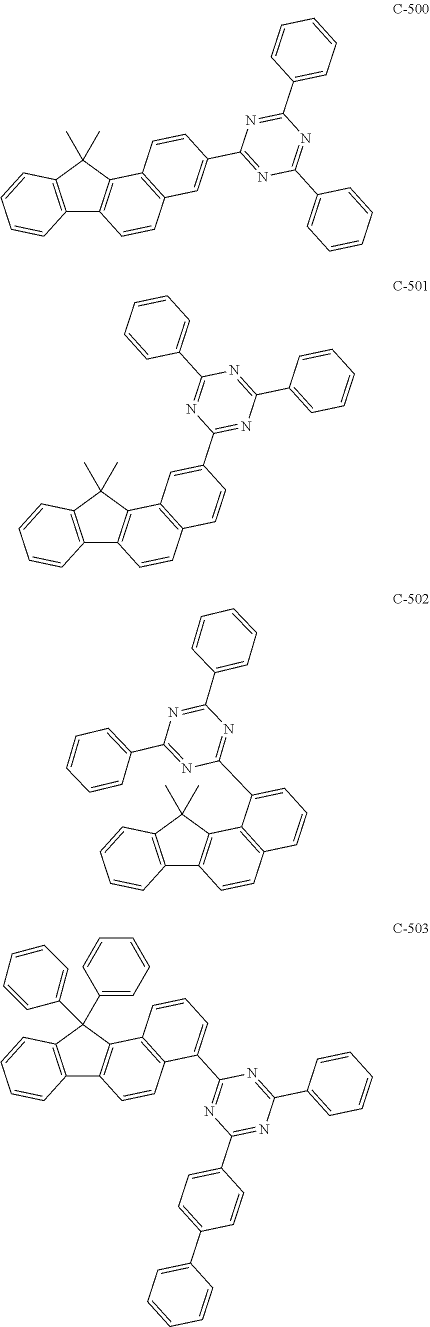

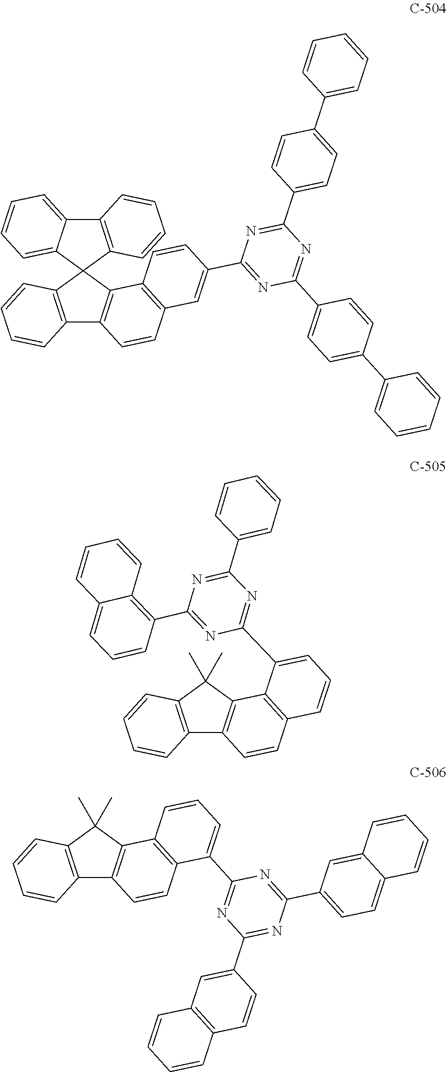

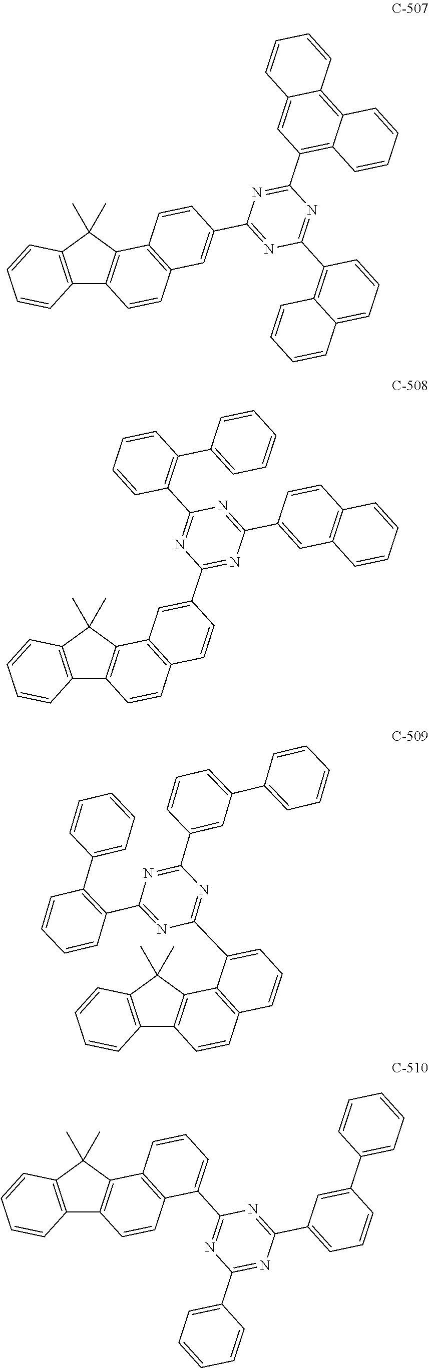 [SMILES: CC1(C)c2ccccc2-c2ccc3cc(-c4nc(-c5cccc6ccccc56)nc(-c5cc6ccccc6c6ccccc56)n4)ccc3c21 ; CC1(C)c2ccccc2-c2ccc3ccc(-c4nc(-c5ccc6ccccc6c5)nc(-c5ccccc5-c5ccccc5)n4)cc3c21 ; CC1(C)c2ccccc2-c2ccc3cccc(-c4nc(-c5cccc(-c6ccccc6)c5)nc(-c5ccccc5-c5ccccc5)n4)c3c21 ; CC1(C)c2ccccc2-c2ccc3c(-c4nc(-c5ccccc5)nc(-c5cccc(-c6ccccc6)c5)n4)cccc3c21]

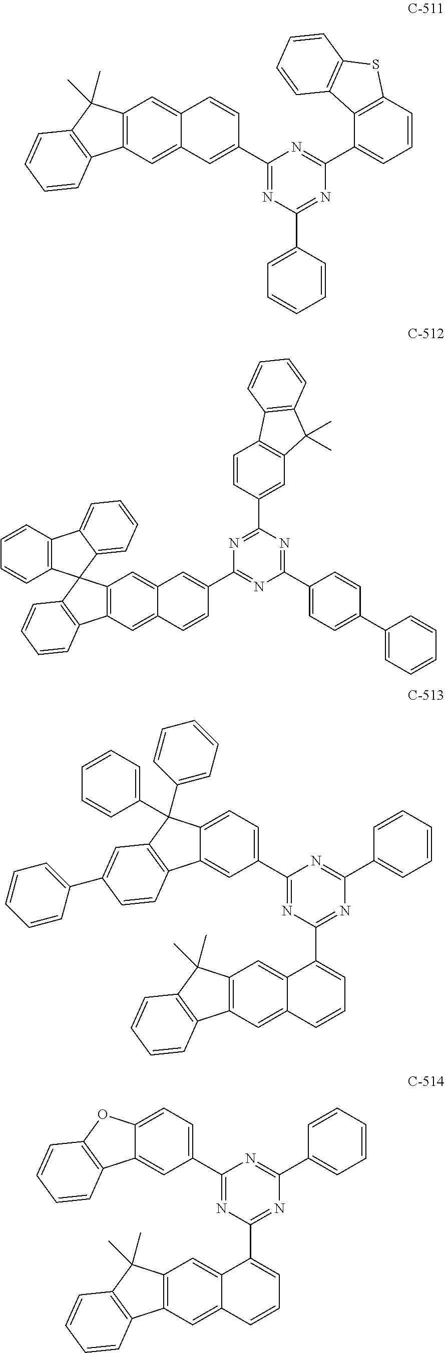

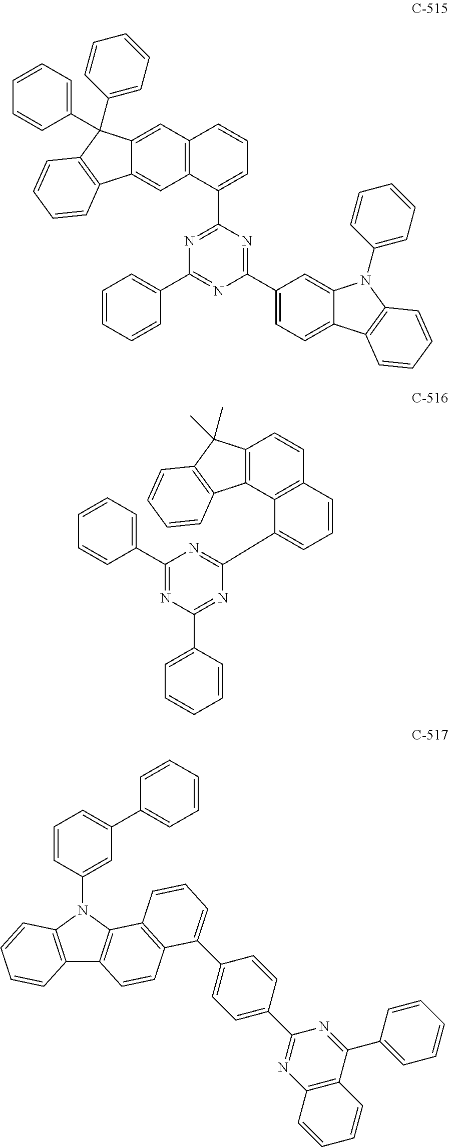 [SMILES: c1ccc(-c2nc(-c3ccc4c5ccccc5n(-c5ccccc5)c4c3)nc(-c3cccc4cc5c(cc34)-c3ccccc3C5(c3ccccc3)c3ccccc3)n2)cc1 ; CC1(C)c2ccccc2-c2c1ccc1cccc(-c3nc(-c4ccccc4)nc(-c4ccccc4)n3)c21 ; c1ccc(-c2cccc(-n3c4ccccc4c4ccc5c(-c6ccc(-c7nc(-c8ccccc8)c8ccccc8n7)cc6)cccc5c43)c2)cc1]

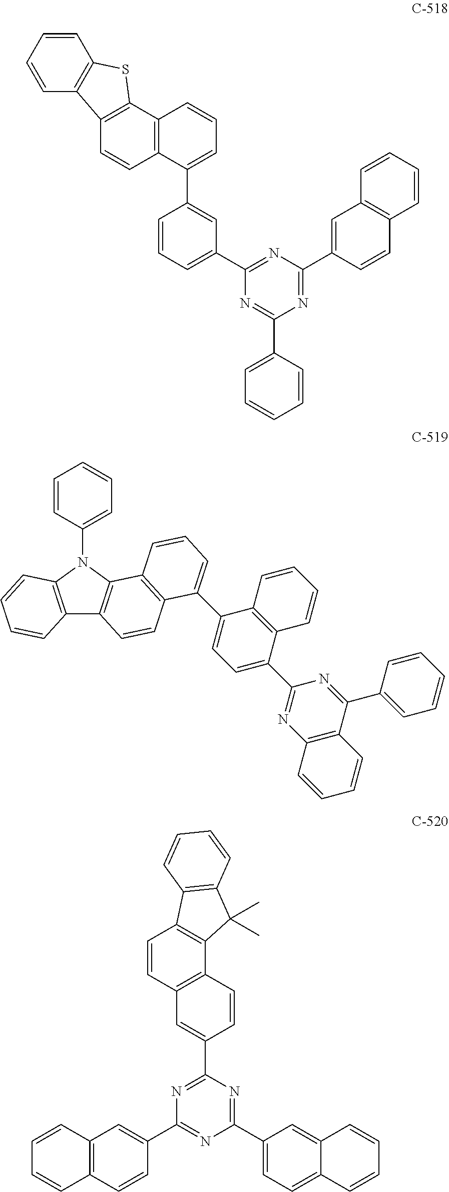 [SMILES: c1ccc(-c2nc(-c3cccc(-c4cccc5c4ccc4c6ccccc6sc54)c3)nc(-c3ccc4ccccc4c3)n2)cc1 ; c1ccc(-c2nc(-c3ccc(-c4cccc5c4ccc4c6ccccc6n(-c6ccccc6)c54)c4ccccc34)nc3ccccc23)cc1 ; CC1(C)c2ccccc2-c2ccc3cc(-c4nc(-c5ccc6ccccc6c5)nc(-c5ccc6ccccc6c5)n4)ccc3c21]

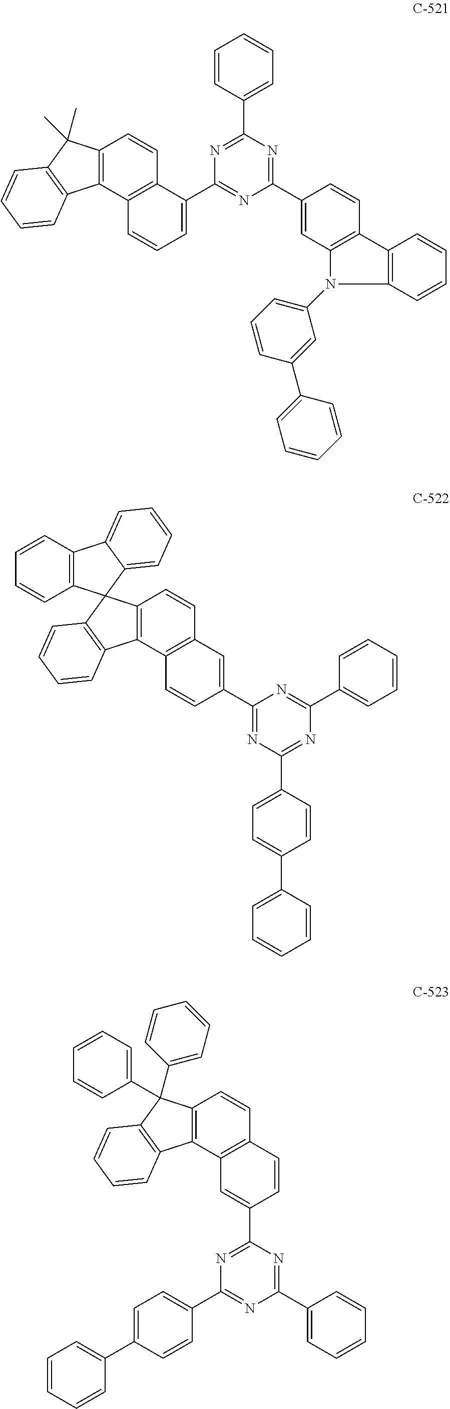 [SMILES: CC1(C)c2ccccc2-c2c1ccc1c(-c3nc(-c4ccccc4)nc(-c4ccc5c6ccccc6n(-c6cccc(-c7ccccc7)c6)c5c4)n3)cccc21 ; c1ccc(-c2ccc(-c3nc(-c4ccccc4)nc(-c4ccc5c6c(ccc5c4)C4(c5ccccc5-c5ccccc54)c4ccccc4-6)n3)cc2)cc1 ; c1ccc(-c2ccc(-c3nc(-c4ccccc4)nc(-c4ccc5ccc6c(c5c4)-c4ccccc4C6(c4ccccc4)c4ccccc4)n3)cc2)cc1]

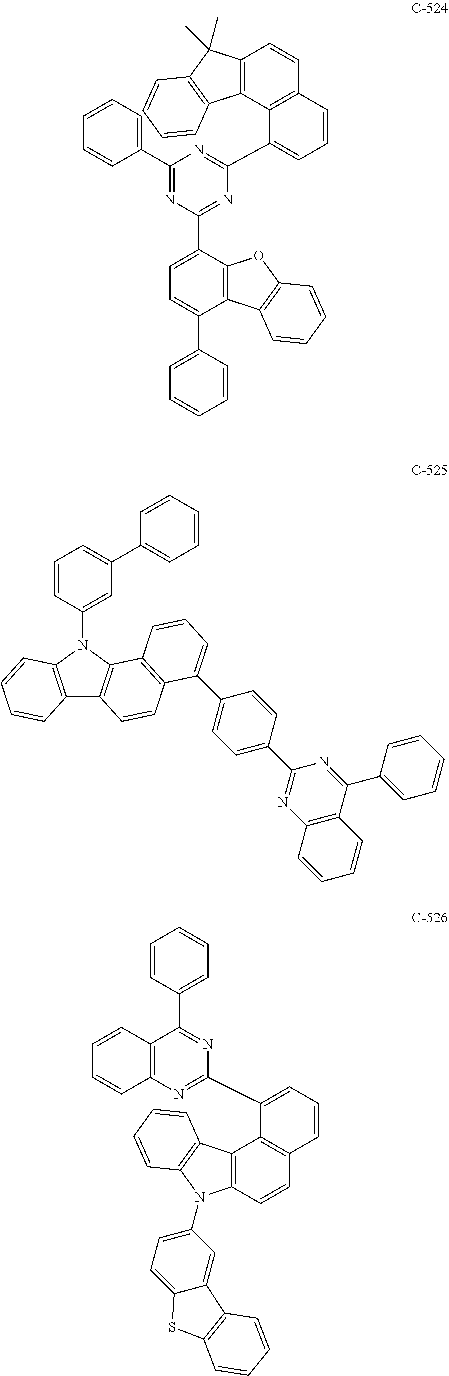 [SMILES: CC1(C)c2ccccc2-c2c1ccc1cccc(-c3nc(-c4ccccc4)nc(-c4ccc(-c5ccccc5)c5c4oc4ccccc45)n3)c21 ; c1ccc(-c2cccc(-n3c4ccccc4c4ccc5c(-c6ccc(-c7nc(-c8ccccc8)c8ccccc8n7)cc6)cccc5c43)c2)cc1 ; c1ccc(-c2nc(-c3cccc4ccc5c(c6ccccc6n5-c5ccc6sc7ccccc7c6c5)c34)nc3ccccc23)cc1]

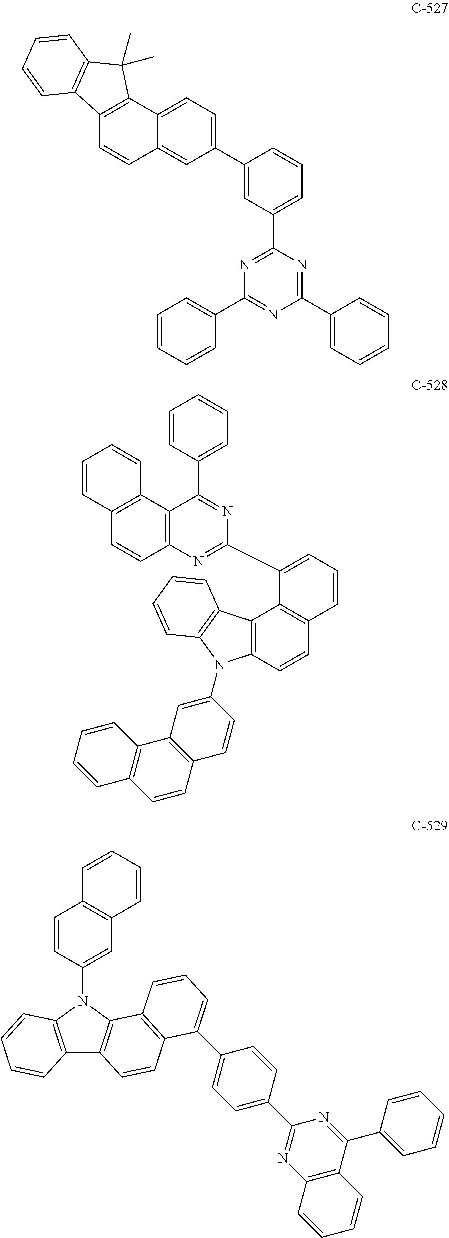

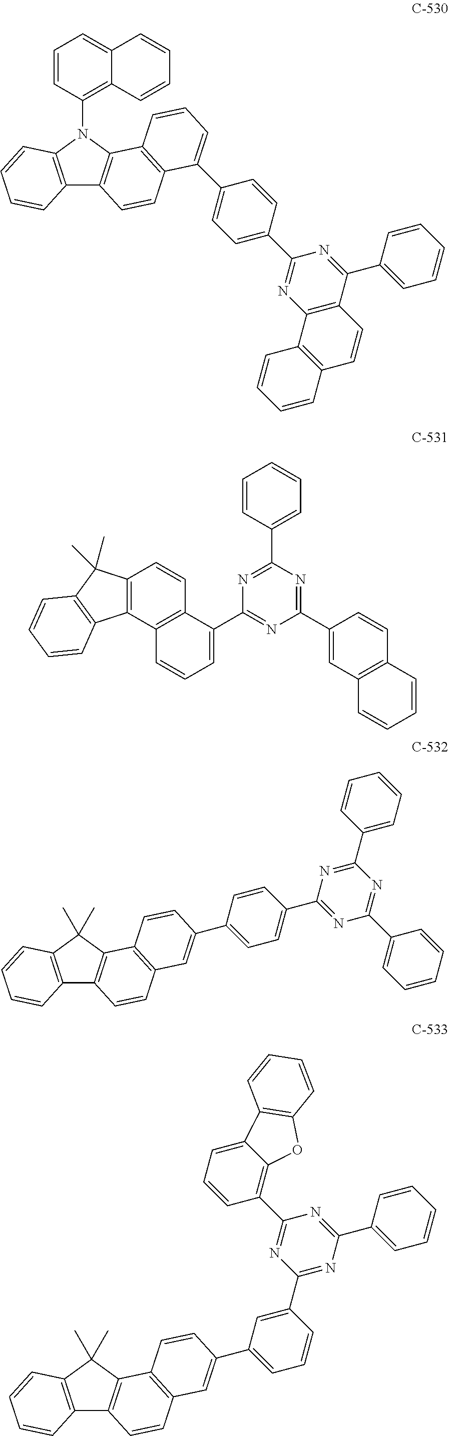

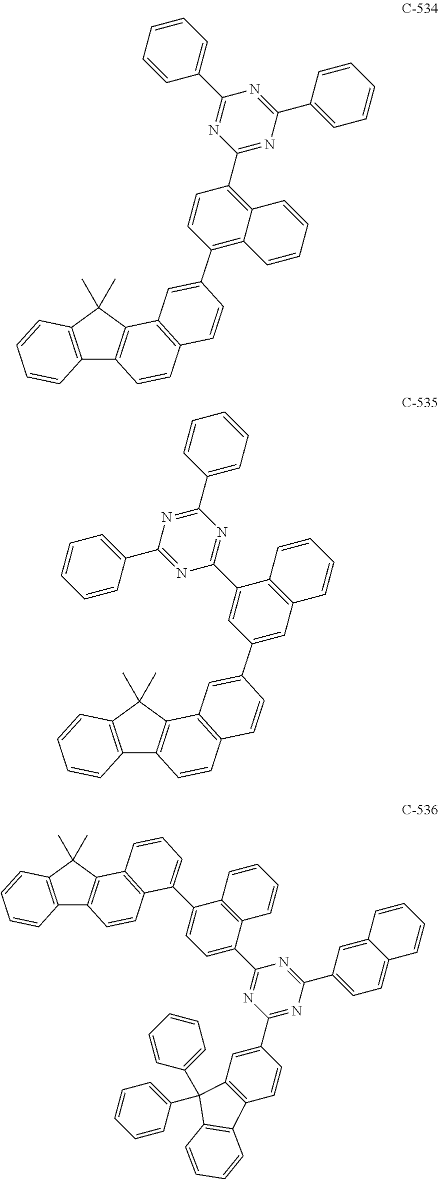 [SMILES: CC1(C)c2ccccc2-c2ccc3ccc(-c4ccc(-c5nc(-c6ccccc6)nc(-c6ccccc6)n5)c5ccccc45)cc3c21 ; CC1(C)c2ccccc2-c2ccc3ccc(-c4cc(-c5nc(-c6ccccc6)nc(-c6ccccc6)n5)c5ccccc5c4)cc3c21 ; CC1(C)c2ccccc2-c2ccc3c(-c4ccc(-c5nc(-c6ccc7c(c6)C(c6ccccc6)(c6ccccc6)c6ccccc6-7)nc(-c6ccc7ccccc7c6)n5)c5ccccc45)cccc3c21]

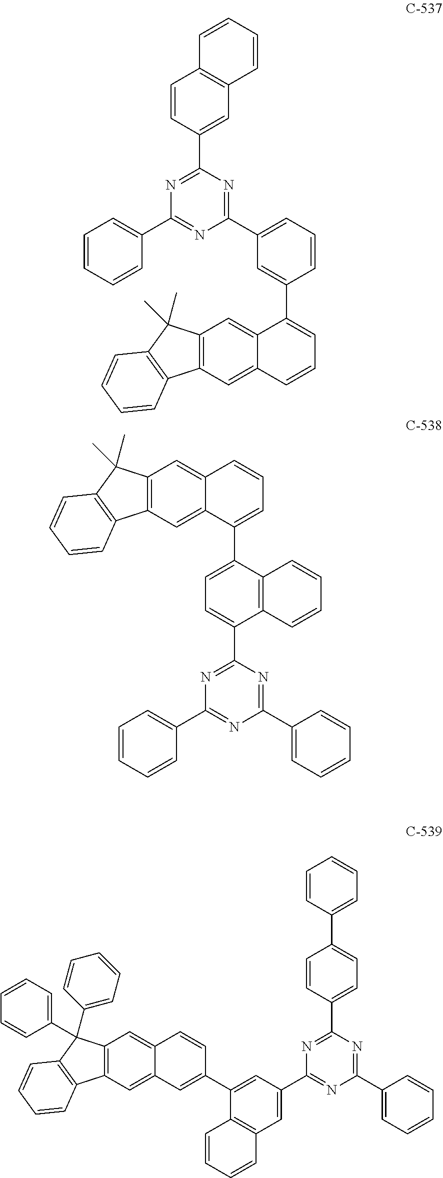

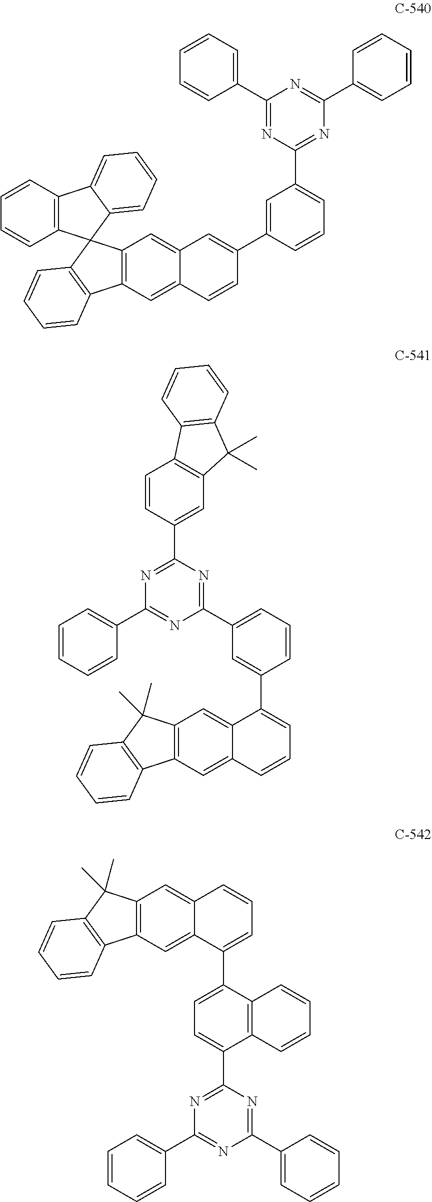

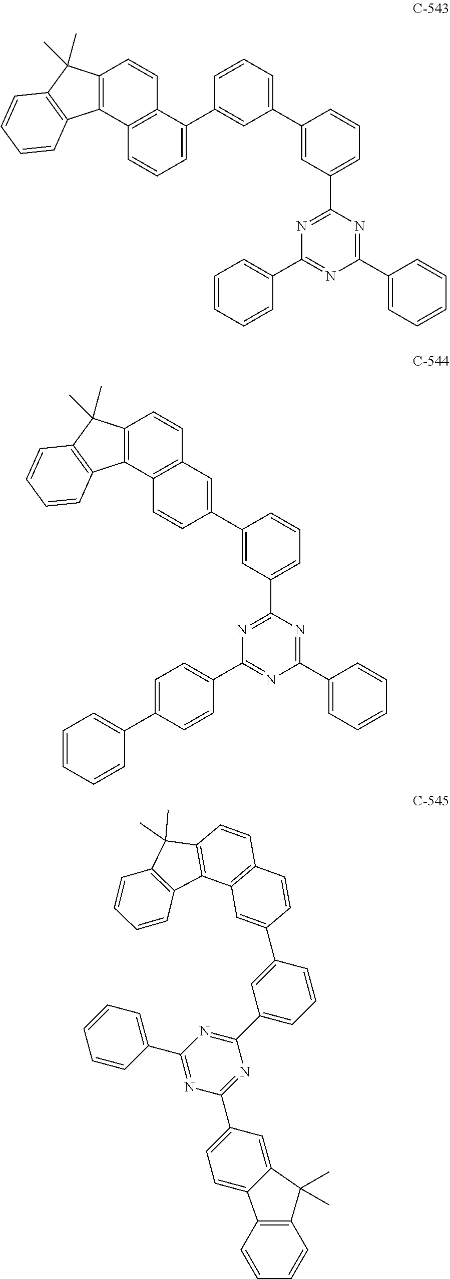 [SMILES: CC1(C)c2ccccc2-c2c1ccc1c(-c3cccc(-c4cccc(-c5nc(-c6ccccc6)nc(-c6ccccc6)n5)c4)c3)cccc21 ; CC1(C)c2ccccc2-c2c1ccc1cc(-c3cccc(-c4nc(-c5ccccc5)nc(-c5ccc(-c6ccccc6)cc5)n4)c3)ccc21 ; CC1(C)c2ccccc2-c2ccc(-c3nc(-c4ccccc4)nc(-c4cccc(-c5ccc6ccc7c(c6c5)-c5ccccc5C7(C)C)c4)n3)cc21]

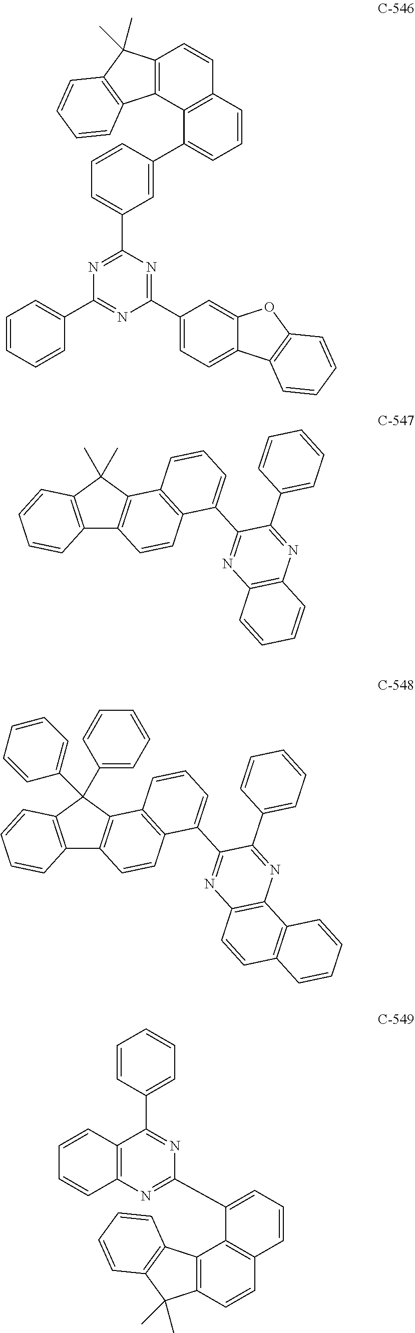

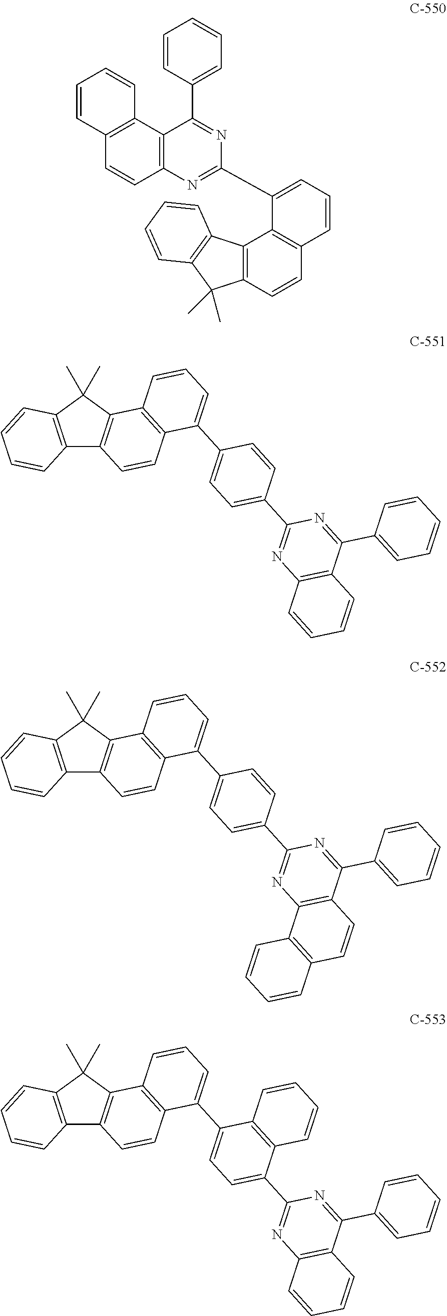 [SMILES: CC1(C)c2ccccc2-c2c1ccc1cccc(-c3nc(-c4ccccc4)c4c(ccc5ccccc54)n3)c21 ; CC1(C)c2ccccc2-c2ccc3c(-c4ccc(-c5nc(-c6ccccc6)c6ccccc6n5)cc4)cccc3c21 ; CC1(C)c2ccccc2-c2ccc3c(-c4ccc(-c5nc(-c6ccccc6)c6ccc7ccccc7c6n5)cc4)cccc3c21 ; CC1(C)c2ccccc2-c2ccc3c(-c4ccc(-c5nc(-c6ccccc6)c6ccccc6n5)c5ccccc45)cccc3c21]

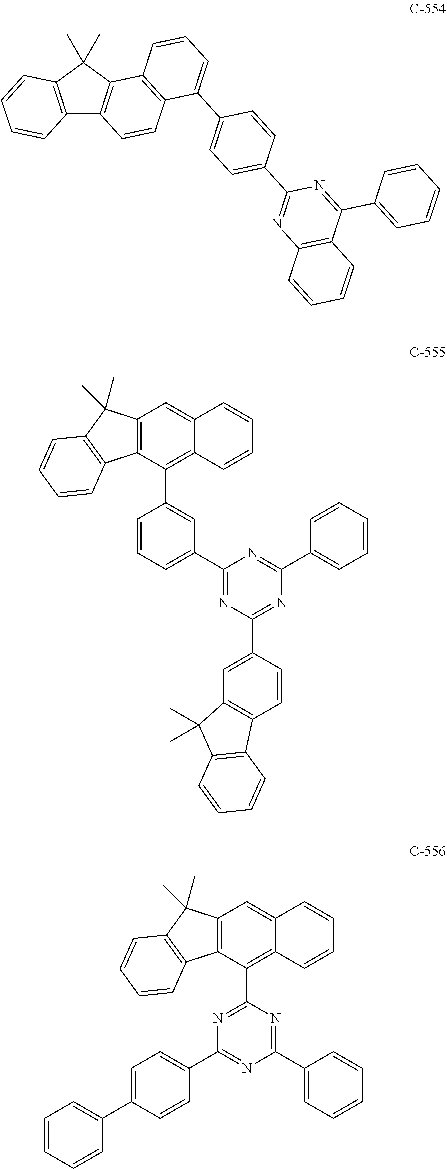 [SMILES: CC1(C)c2ccccc2-c2ccc3c(-c4ccc(-c5nc(-c6ccccc6)c6ccccc6n5)cc4)cccc3c21 ; CC1(C)c2ccccc2-c2ccc(-c3nc(-c4ccccc4)nc(-c4cccc(-c5c6c(cc7ccccc57)C(C)(C)c5ccccc5-6)c4)n3)cc21 ; CC1(C)c2ccccc2-c2c1cc1ccccc1c2-c1nc(-c2ccccc2)nc(-c2ccc(-c3ccccc3)cc2)n1]

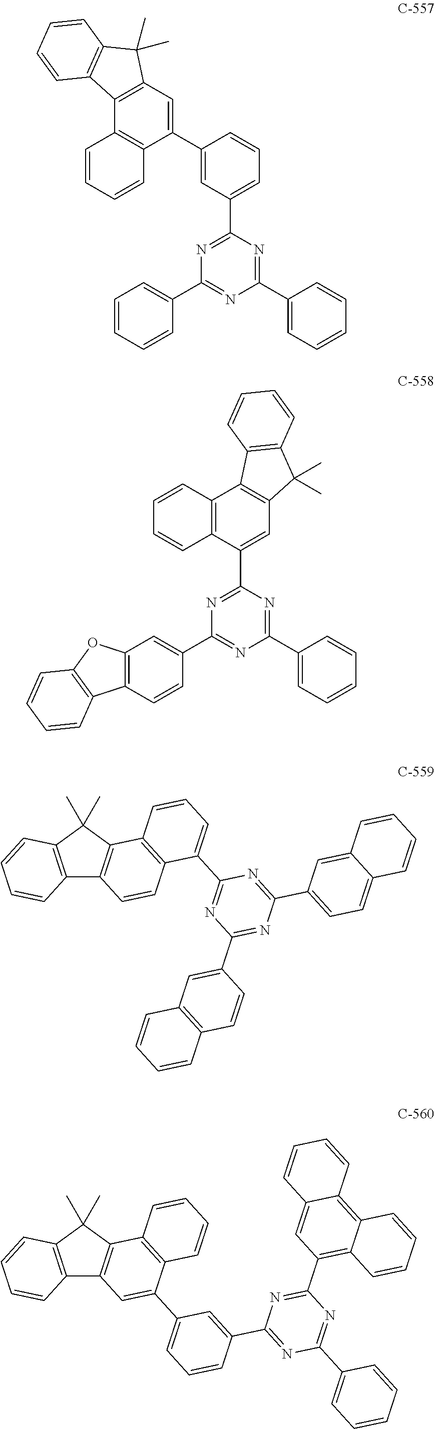 [SMILES: CC1(C)c2ccccc2-c2c1cc(-c1cccc(-c3nc(-c4ccccc4)nc(-c4ccccc4)n3)c1)c1ccccc21 ; CC1(C)c2ccccc2-c2c1cc(-c1nc(-c3ccccc3)nc(-c3ccc4c(c3)oc3ccccc34)n1)c1ccccc21 ; CC1(C)c2ccccc2-c2ccc3c(-c4nc(-c5ccc6ccccc6c5)nc(-c5ccc6ccccc6c5)n4)cccc3c21 ; CC1(C)c2ccccc2-c2cc(-c3cccc(-c4nc(-c5ccccc5)nc(-c5cc6ccccc6c6ccccc56)n4)c3)c3ccccc3c21]

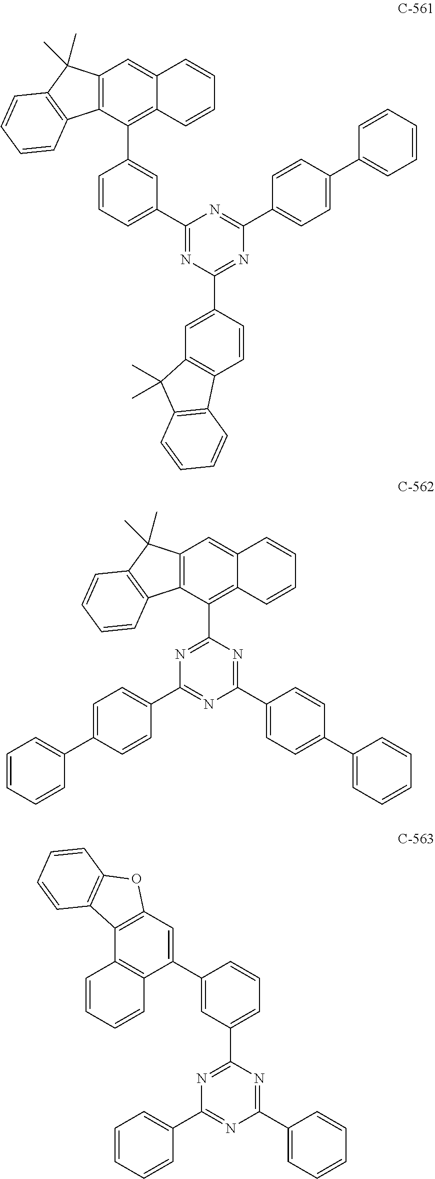 [SMILES: CC1(C)c2ccccc2-c2ccc(-c3nc(-c4ccc(-c5ccccc5)cc4)nc(-c4cccc(-c5c6c(cc7ccccc57)C(C)(C)c5ccccc5-6)c4)n3)cc21 ; CC1(C)c2ccccc2-c2c1cc1ccccc1c2-c1nc(-c2ccc(-c3ccccc3)cc2)nc(-c2ccc(-c3ccccc3)cc2)n1 ; c1ccc(-c2nc(-c3ccccc3)nc(-c3cccc(-c4cc5oc6ccccc6c5c5ccccc45)c3)n2)cc1]

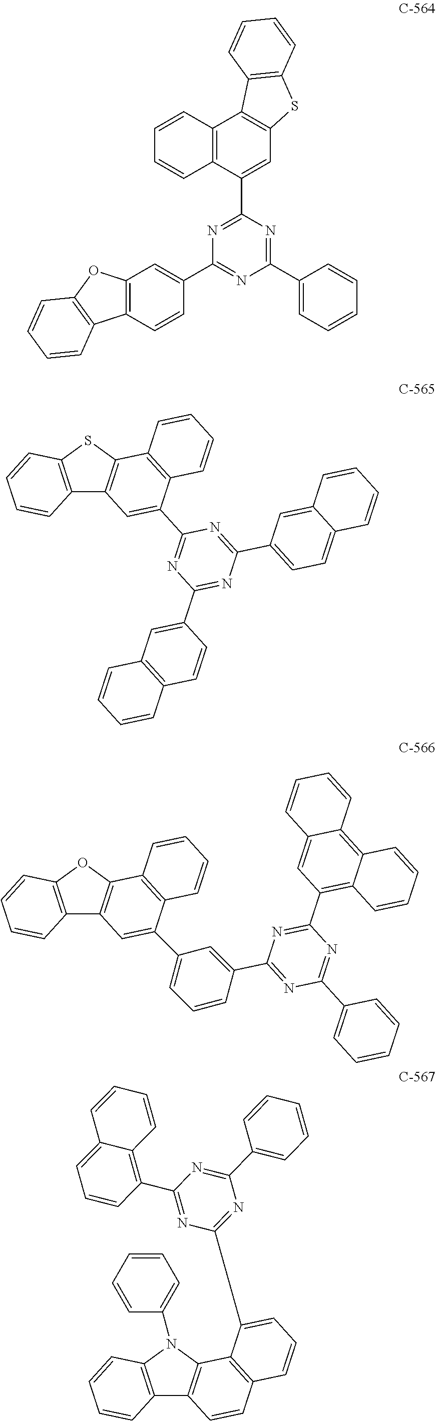

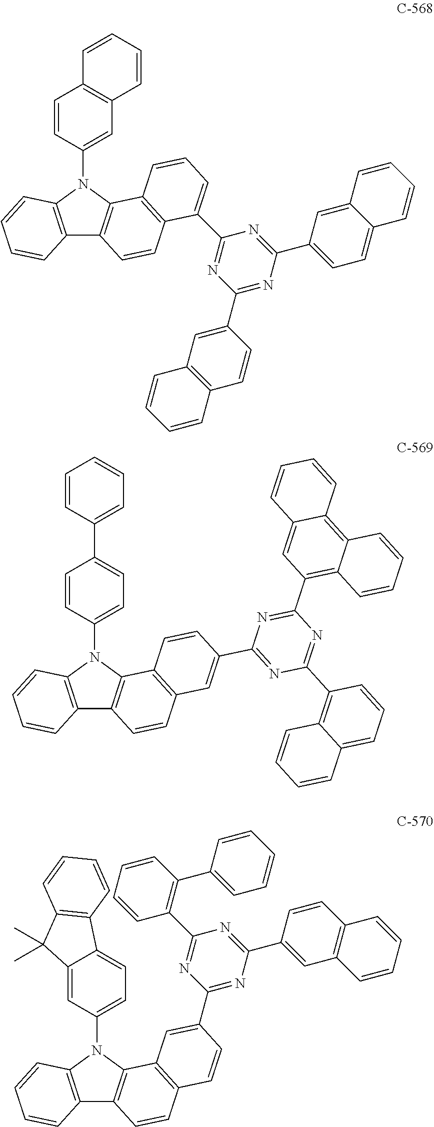 [SMILES: c1ccc2cc(-c3nc(-c4ccc5ccccc5c4)nc(-c4cccc5c4ccc4c6ccccc6n(-c6ccc7ccccc7c6)c54)n3)ccc2c1 ; c1ccc(-c2ccc(-n3c4ccccc4c4ccc5cc(-c6nc(-c7cccc8ccccc78)nc(-c7cc8ccccc8c8ccccc78)n6)ccc5c43)cc2)cc1 ; CC1(C)c2ccccc2-c2ccc(-n3c4ccccc4c4ccc5ccc(-c6nc(-c7ccc8ccccc8c7)nc(-c7ccccc7-c7ccccc7)n6)cc5c43)cc21]

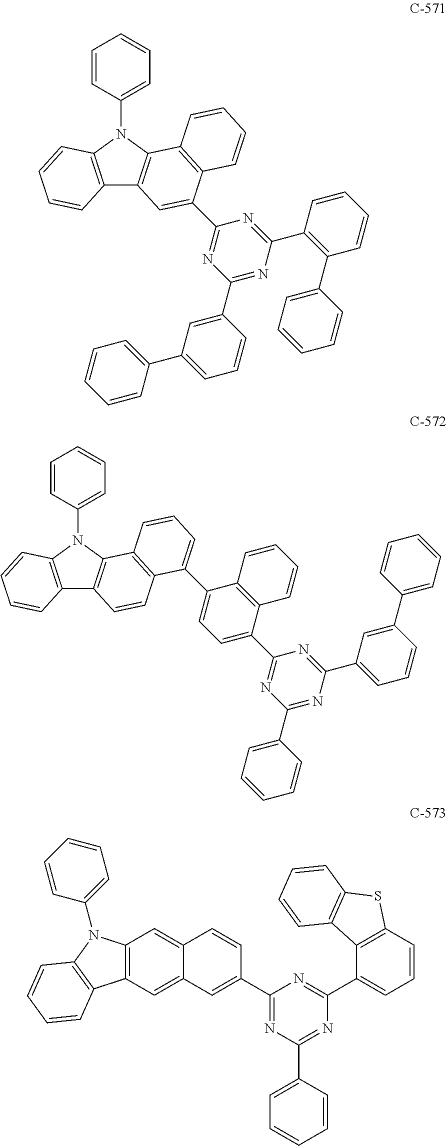 [SMILES: c1ccc(-c2cccc(-c3nc(-c4ccccc4-c4ccccc4)nc(-c4cc5c6ccccc6n(-c6ccccc6)c5c5ccccc45)n3)c2)cc1 ; c1ccc(-c2cccc(-c3nc(-c4ccccc4)nc(-c4ccc(-c5cccc6c5ccc5c7ccccc7n(-c7ccccc7)c65)c5ccccc45)n3)c2)cc1 ; c1ccc(-c2nc(-c3ccc4cc5c(cc4c3)c3ccccc3n5-c3ccccc3)nc(-c3cccc4sc5ccccc5c34)n2)cc1]

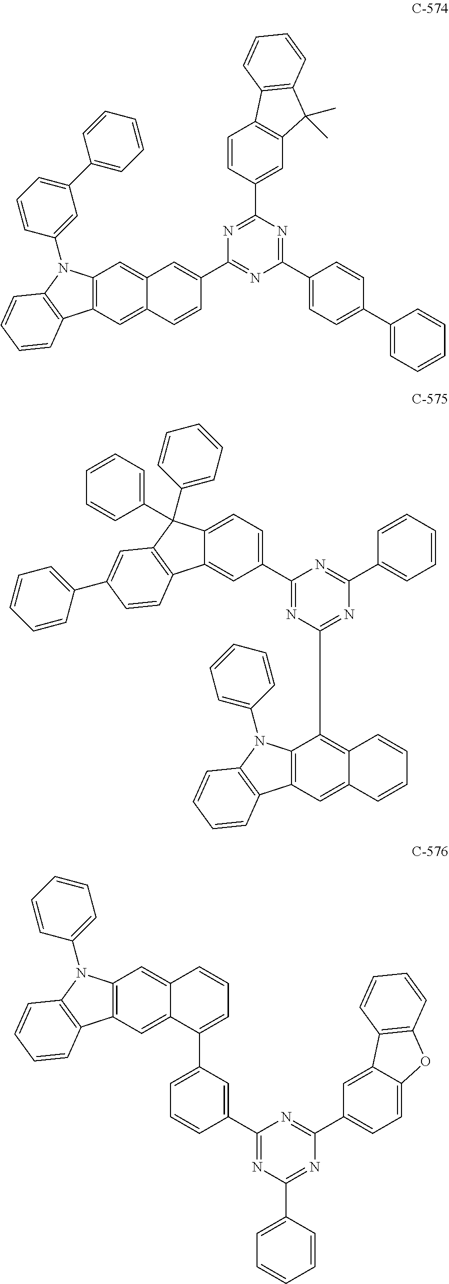 [SMILES: CC1(C)c2ccccc2-c2ccc(-c3nc(-c4ccc(-c5ccccc5)cc4)nc(-c4ccc5cc6c7ccccc7n(-c7cccc(-c8ccccc8)c7)c6cc5c4)n3)cc21 ; c1ccc(-c2ccc3c(c2)C(c2ccccc2)(c2ccccc2)c2ccc(-c4nc(-c5ccccc5)nc(-c5c6ccccc6cc6c7ccccc7n(-c7ccccc7)c56)n4)cc2-3)cc1 ; c1ccc(-c2nc(-c3cccc(-c4cccc5cc6c(cc45)c4ccccc4n6-c4ccccc4)c3)nc(-c3ccc4oc5ccccc5c4c3)n2)cc1]

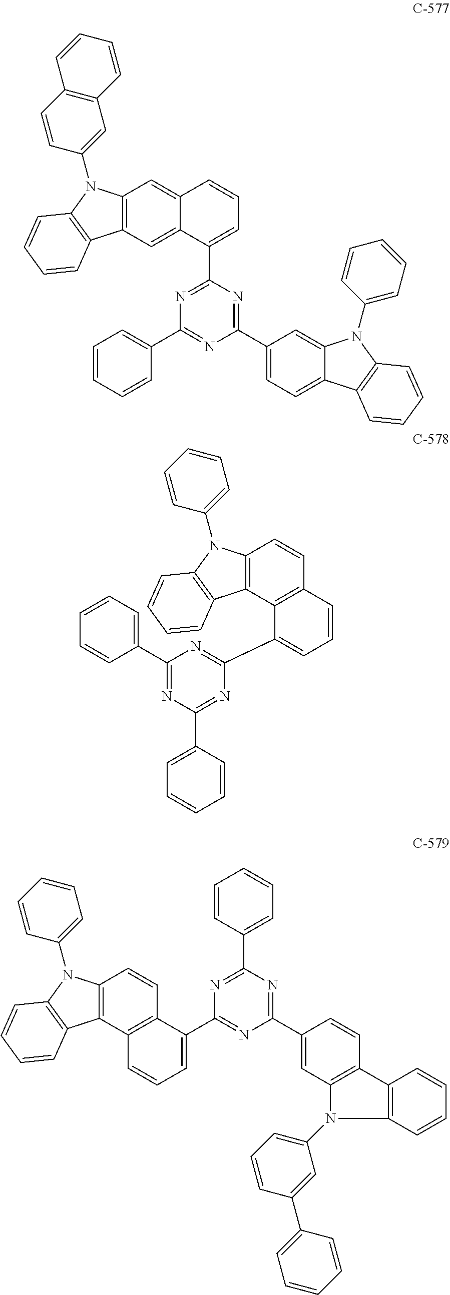 [SMILES: c1ccc(-c2nc(-c3ccc4c5ccccc5n(-c5ccccc5)c4c3)nc(-c3cccc4cc5c(cc34)c3ccccc3n5-c3ccc4ccccc4c3)n2)cc1 ; c1ccc(-c2nc(-c3ccccc3)nc(-c3cccc4ccc5c(c6ccccc6n5-c5ccccc5)c34)n2)cc1 ; c1ccc(-c2cccc(-n3c4ccccc4c4ccc(-c5nc(-c6ccccc6)nc(-c6cccc7c6ccc6c7c7ccccc7n6-c6ccccc6)n5)cc43)c2)cc1]

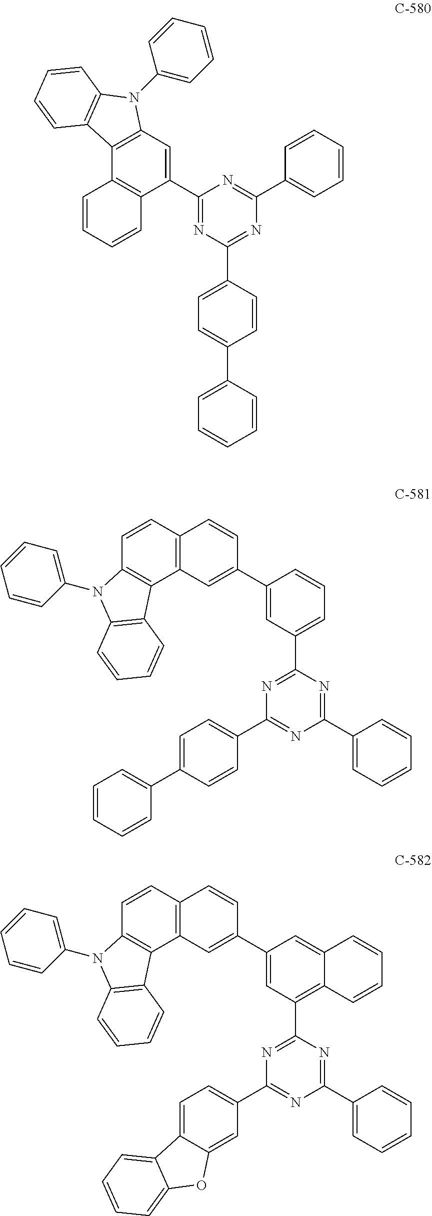 [SMILES: c1ccc(-c2ccc(-c3nc(-c4ccccc4)nc(-c4cc5c(c6ccccc46)c4ccccc4n5-c4ccccc4)n3)cc2)cc1 ; c1ccc(-c2ccc(-c3nc(-c4ccccc4)nc(-c4cccc(-c5ccc6ccc7c(c6c5)c5ccccc5n7-c5ccccc5)c4)n3)cc2)cc1 ; c1ccc(-c2nc(-c3ccc4c(c3)oc3ccccc34)nc(-c3cc(-c4ccc5ccc6c(c5c4)c4ccccc4n6-c4ccccc4)cc4ccccc34)n2)cc1]

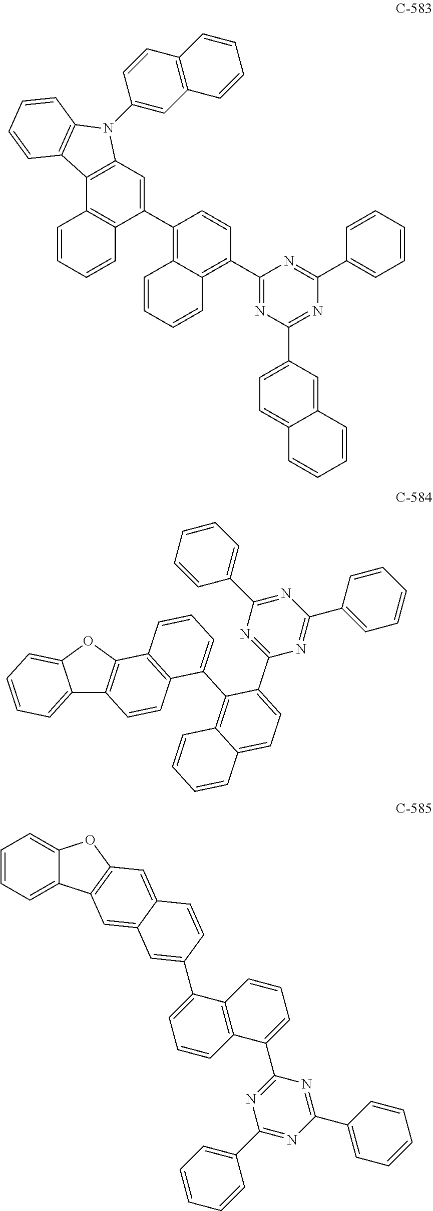 [SMILES: c1ccc(-c2nc(-c3ccc4ccccc4c3)nc(-c3ccc(-c4cc5c(c6ccccc46)c4ccccc4n5-c4ccc5ccccc5c4)c4ccccc34)n2)cc1 ; c1ccc(-c2nc(-c3ccccc3)nc(-c3ccc4ccccc4c3-c3cccc4c3ccc3c5ccccc5oc43)n2)cc1 ; c1ccc(-c2nc(-c3ccccc3)nc(-c3cccc4c(-c5ccc6cc7oc8ccccc8c7cc6c5)cccc34)n2)cc1]

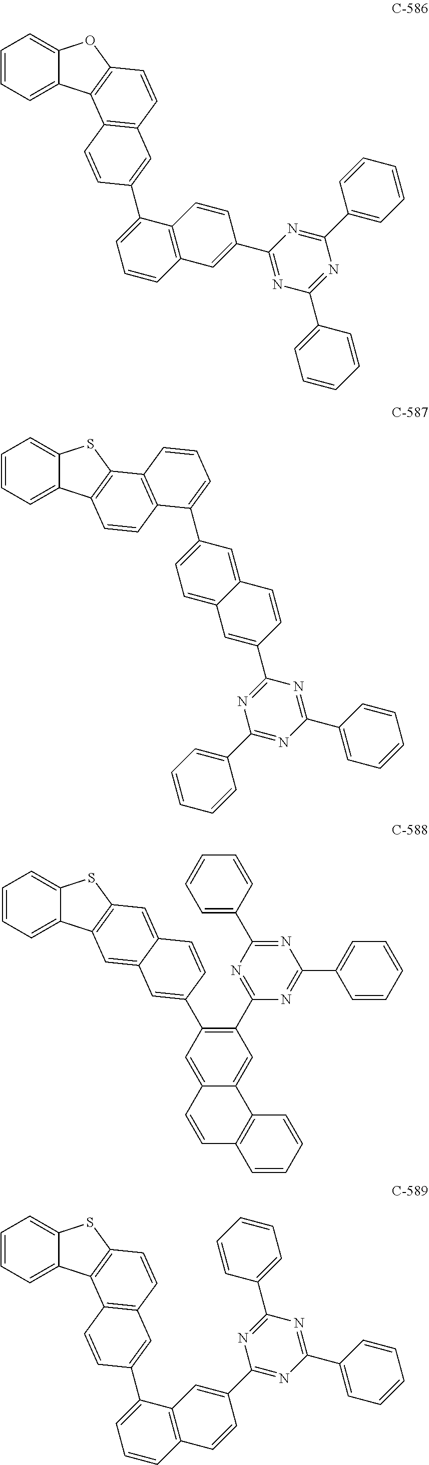

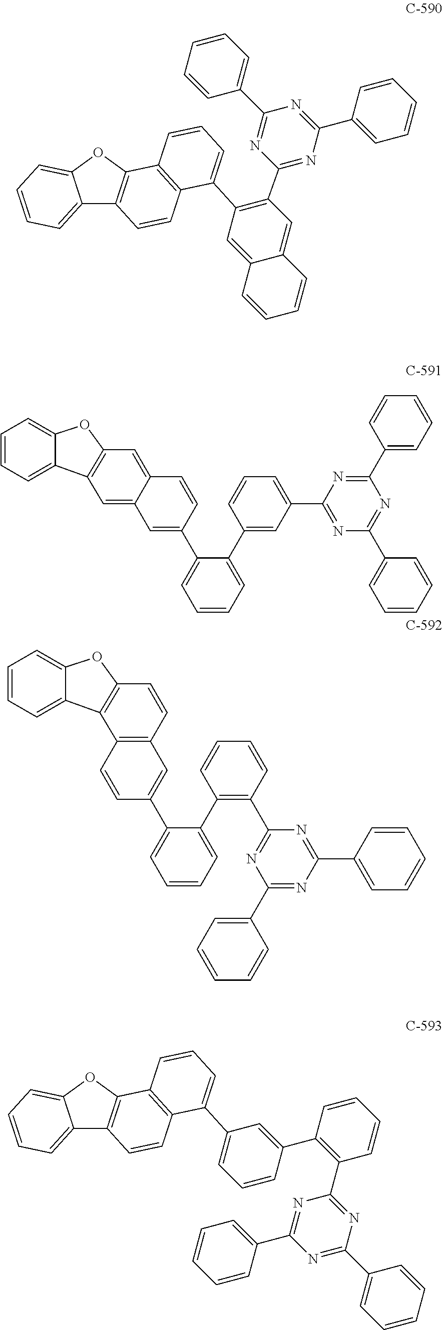 [SMILES: c1ccc(-c2nc(-c3ccccc3)nc(-c3cc4ccccc4cc3-c3cccc4c3ccc3c5ccccc5oc43)n2)cc1 ; c1ccc(-c2nc(-c3ccccc3)nc(-c3cccc(-c4ccccc4-c4ccc5cc6oc7ccccc7c6cc5c4)c3)n2)cc1 ; c1ccc(-c2nc(-c3ccccc3)nc(-c3ccccc3-c3ccccc3-c3ccc4c(ccc5oc6ccccc6c54)c3)n2)cc1 ; c1ccc(-c2nc(-c3ccccc3)nc(-c3ccccc3-c3cccc(-c4cccc5c4ccc4c6ccccc6oc54)c3)n2)cc1]

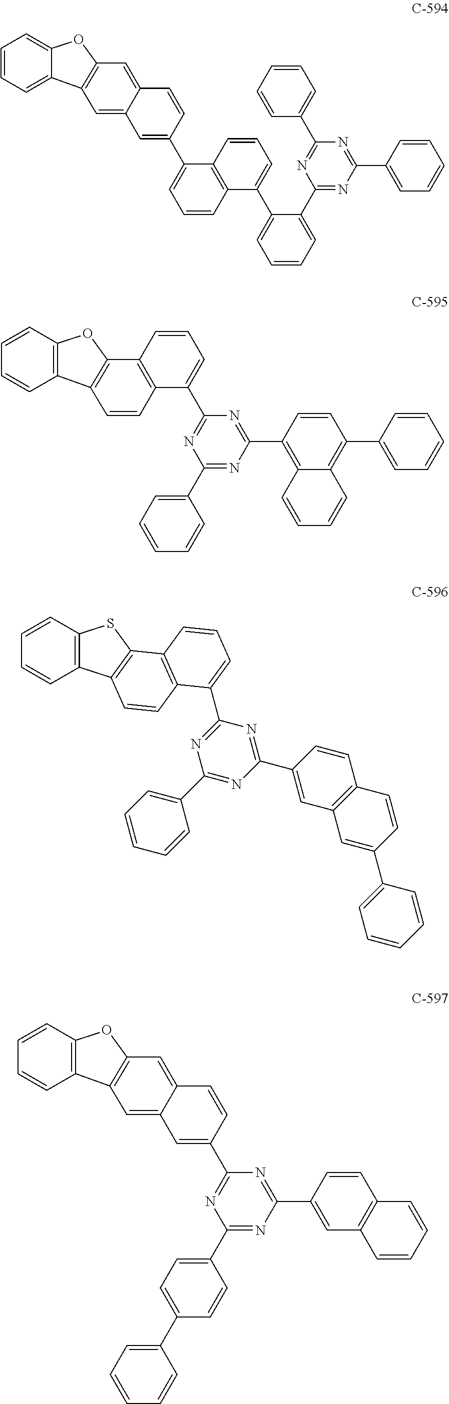 [SMILES: c1ccc(-c2nc(-c3ccccc3)nc(-c3ccccc3-c3cccc4c(-c5ccc6cc7oc8ccccc8c7cc6c5)cccc34)n2)cc1 ; c1ccc(-c2nc(-c3ccc(-c4ccccc4)c4ccccc34)nc(-c3cccc4c3ccc3c5ccccc5oc43)n2)cc1 ; c1ccc(-c2ccc3ccc(-c4nc(-c5ccccc5)nc(-c5cccc6c5ccc5c7ccccc7sc65)n4)cc3c2)cc1 ; c1ccc(-c2ccc(-c3nc(-c4ccc5ccccc5c4)nc(-c4ccc5cc6oc7ccccc7c6cc5c4)n3)cc2)cc1]

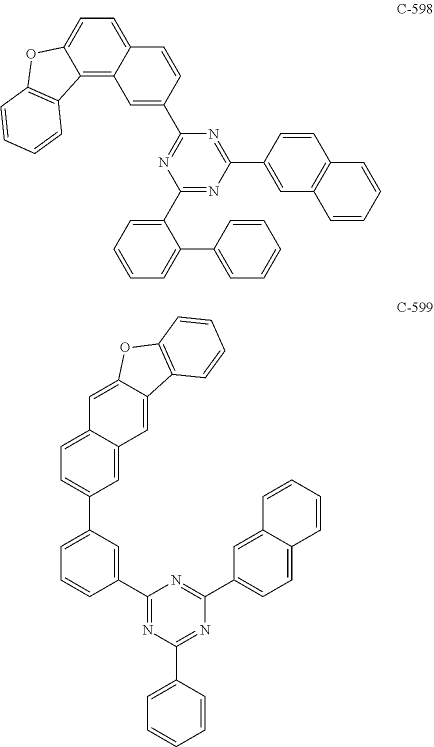

At least one of compounds H-1-1 to H-1-185 and at least one of compounds C-1 to C-599 may be combined and used in an organic electroluminescent device.

The compound represented by formula 1 of the present disclosure may be produced by a synthetic method known to one skilled in the art, and for example, according to the methods disclosed in Korean Patent Application Laying-Open Nos. 2014-0097044 (Aug. 6, 2014), 2013-0106255 (Sep. 27, 2013), 2018-0099510 (Sep. 5, 2018), and 2018-0012709 (Feb. 6, 2018), Korean Patent Application No. 10-2012-0099963 (Sep. 10, 2012), etc.

The compound represented by formula 2 of the present disclosure may be produced by a synthetic method known to one skilled in the art, and for example, according to the following reaction schemes 1 to 4, but is not limited thereto:

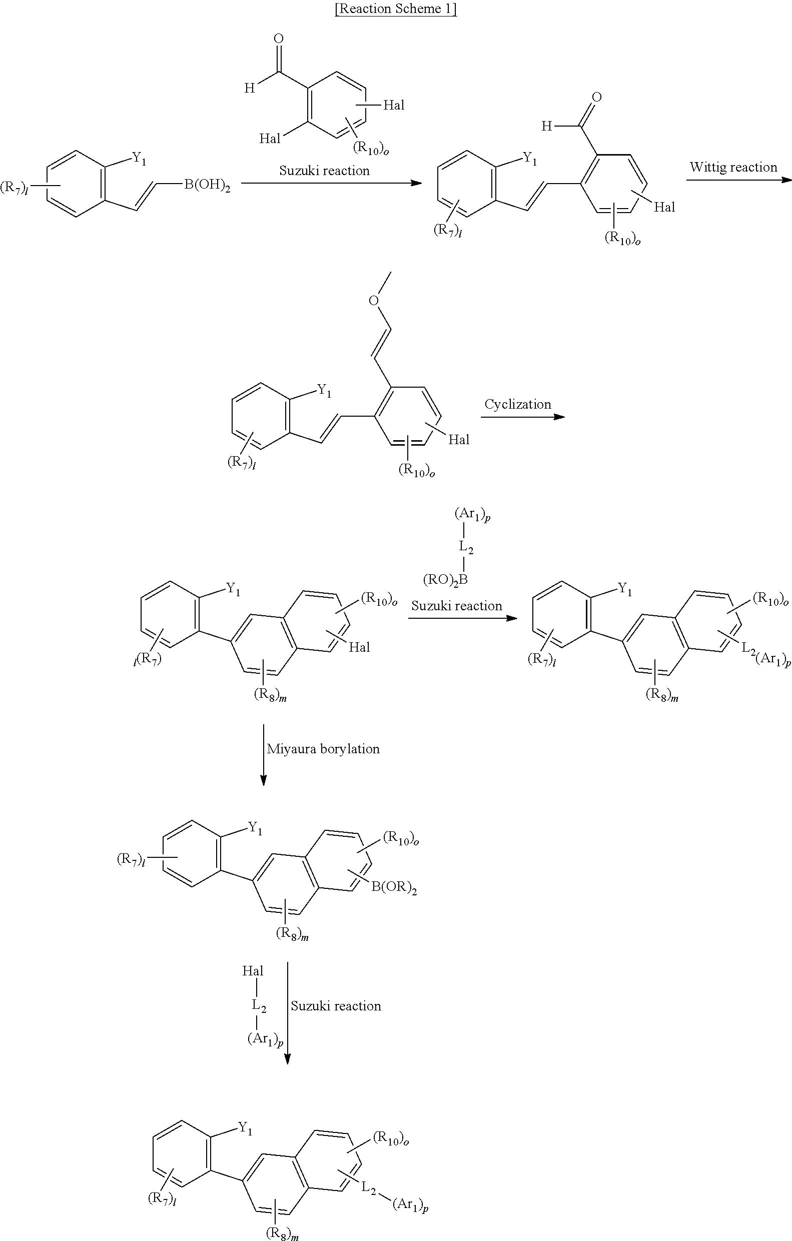

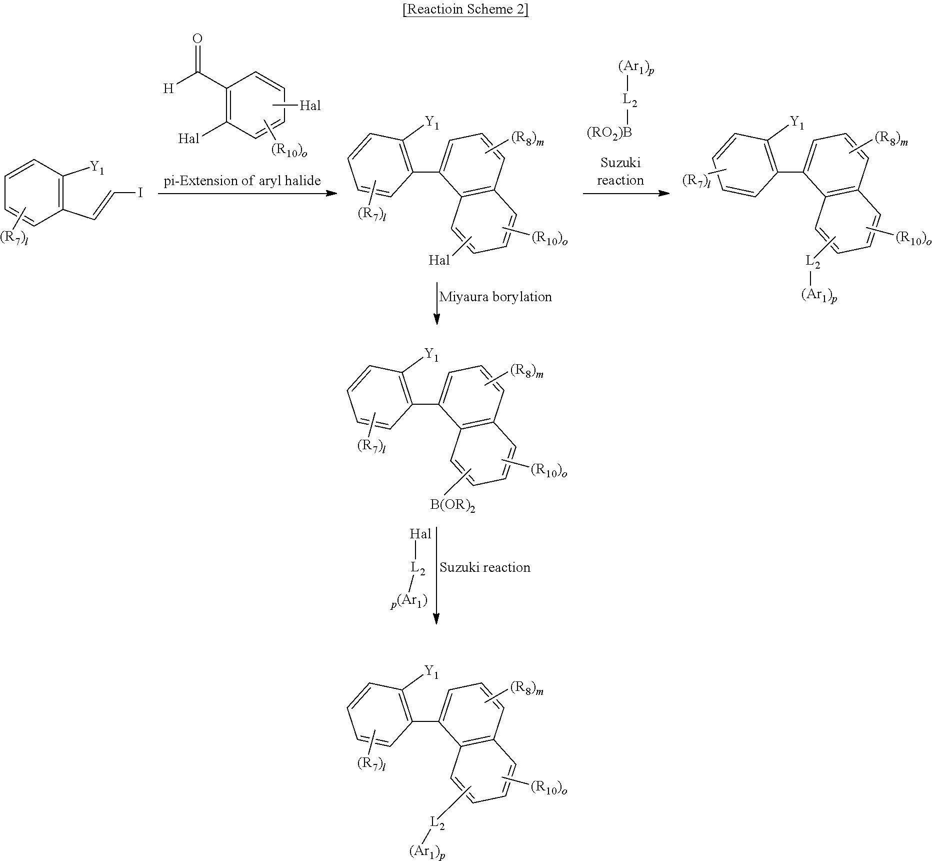

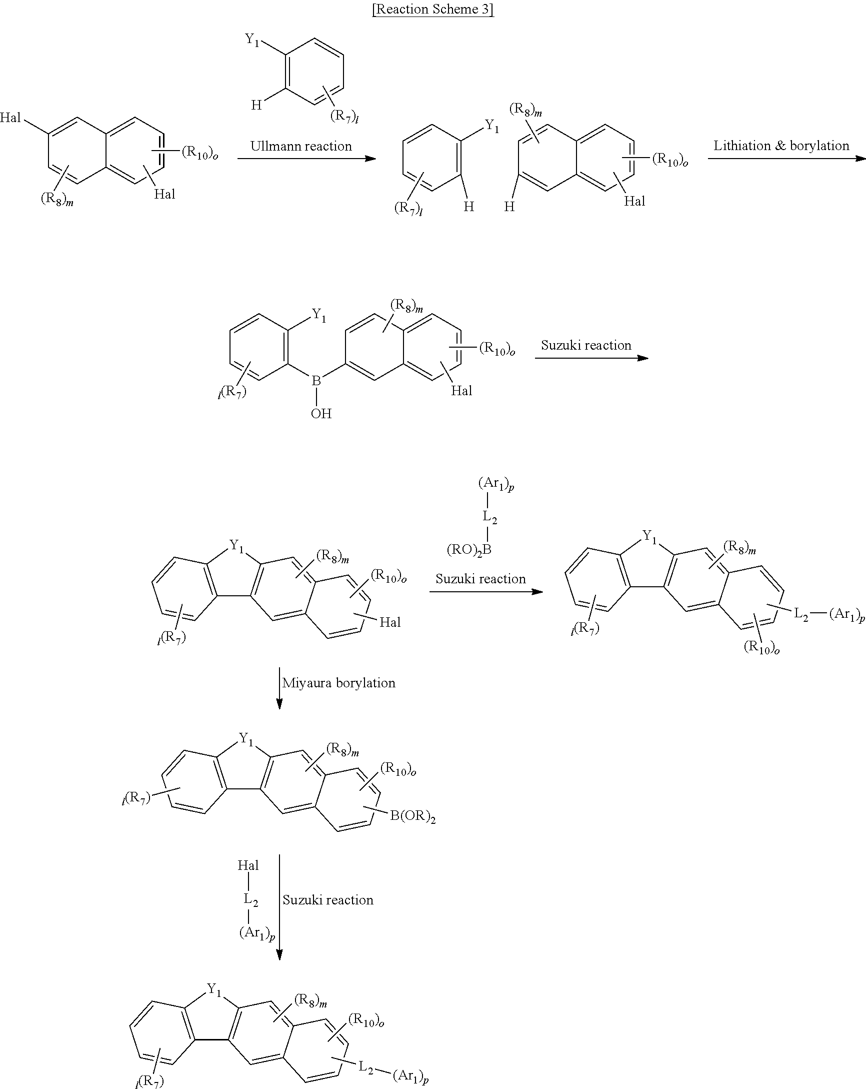

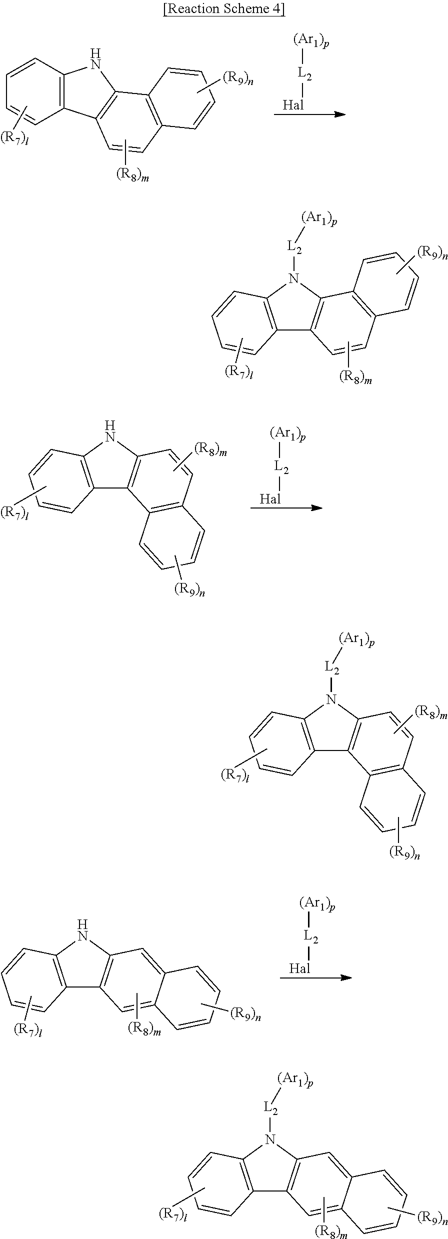

In reaction schemes 1 to 4, the respective substituents are as defined in formulas 2-1 to 2-9; and Hal represents a halogen.

Although illustrative synthesis examples of the compound represented by formula 2, specifically the compounds represented by formulas 2-1 to 2-9, are described above, one skilled in the art will be able to readily understand that all of them are based on a Suzuki cross-coupling reaction, Wittig reaction, a Miyaura borylation reaction, an Ullmann reaction, a Buchwald-Hartwig cross-coupling reaction, an N-arylation reaction, an H-mont-mediated etherification reaction, an Intramolecular acid-induced cyclization reaction, a Pd(II)-catalyzed oxidative cyclization reaction, a Grignard reaction, a Heck reaction, a Cyclic Dehydration reaction, an SN₁ substitution reaction, an SN₂ substitution reaction, a Phosphine-mediated reductive cyclization reaction, etc., and the reactions above proceed even when substituents which are defined in formulas 2-1 to 2-9 above, but are not specified in the specific synthesis examples, are bonded.

The organic electroluminescent device of the present disclosure may comprise a first electrode, a second electrode, and at least one organic layer between the first and second electrodes.

One of the first and second electrodes may be an anode, and the other may be a cathode. The organic layer may comprise a light-emitting layer, and may further comprise at least one layer selected from a hole injection layer, a hole transport layer, a hole auxiliary layer, a light-emitting auxiliary layer, an electron transport layer, an electron buffer layer, an electron injection layer, an interlayer, a hole blocking layer, and an electron blocking layer. The second electrode may be a transflective electrode or a reflective electrode and may be a top emission type, a bottom emission type, or both-sides emission type according to the kinds of the material. In addition, the hole injection layer may be further doped with a p-dopant and the electron injection layer may also be further doped with an n-dopant.

The organic electroluminescent device according to the present disclosure may comprise an anode, a cathode, and at least one organic layer between the anode and cathode in which the organic layer may comprise a plurality of organic electroluminescent materials, including the compound represented by formula 1 as the first organic electroluminescent material, and the compound represented by formula 2 as the second organic electroluminescent material. According to one embodiment of the present disclosure, the organic electroluminescent device according to the present disclosure may comprise an anode, a cathode, and at least one light-emitting layer between the anode and cathode in which the light-emitting layer may comprise the compound represented by formula 1 and the compound represented by formula 2.

The light-emitting layer includes a host and a dopant, in which the host includes a plurality of host materials and the compound represented by formula 1 may be included as the first host compound of the plurality of host materials, and the compound represented by formula 2 may be included as the second host compound of the plurality of host materials.

The weight ratio of the first host compound and the second host compound is about 1:99 to about 99:1, preferably about 10:90 to about 90:10, more preferably about 30:70 to about 70:30, even more preferably about 40:60 to about 60:40, and most preferably about 50:50.

Herein, the light-emitting layer is a layer from which light is emitted, and may be a single layer or a multi-layer of which two or more layers are stacked. All of the first host material and the second host material may be included in one layer, or the first host material and the second host material may be included in respective different light-emitting layers. According to one embodiment of the present disclosure, the doping concentration of the dopant compound with respect to the host compound in the light-emitting layer may be less than 20 wt %.

The organic electroluminescent device of the present disclosure may further comprise at least one layer selected from a hole injection layer, a hole transport layer, a hole auxiliary layer, a light-emitting auxiliary layer, an electron transport layer, an electron injection layer, an interlayer, an electron buffer layer, a hole blocking layer, and an electron blocking layer. According to one embodiment of the present disclosure, the organic electroluminescent device of the present disclosure may further comprise an amine-based compound besides the plurality of host materials of the present disclosure as at least one of a hole injection material, a hole transport material, a hole auxiliary material, a light-emitting material, a light-emitting auxiliary material, and an electron blocking material. Further, according to one embodiment of the present disclosure, the organic electroluminescent device of the present disclosure may further comprise an azine-based compound besides the plurality of host materials of the present disclosure as at least one of an electron transport material, an electron injection material, an electron buffer material, and a hole blocking material.

The plurality of host materials according to the present disclosure may be used as light-emitting materials for a white organic light-emitting device. The white organic light-emitting device has been suggested to have various structures such as a parallel arrangement (side-by-side) method, a stacking method, or color conversion material (CCM) method, etc., according to the arrangement of R (red), G (green) or YG (yellowish green), and B (blue) light-emitting units. In addition, the plurality of host materials according to an embodiment of the present disclosure may also be used in an organic electroluminescent device comprising a quantum dot (QD).

A hole injection layer, a hole transport layer, an electron blocking layer, or a combination thereof can be used between the anode and the light-emitting layer. The hole injection layer may be multilayers in order to lower the hole injection barrier (or hole injection voltage) from the anode to the hole transport layer or the electron blocking layer, wherein each of the multilayers may use two compounds simultaneously. In addition, the electron blocking layer may be placed between the hole transport layer (or hole injection layer) and the light-emitting layer, and may block overflow of electrons from the light-emitting layer and confine the excitons in the light-emitting layer to prevent light leakage. The hole transport layer or the electron blocking layer may also be multilayers, wherein each of the multilayers may use a plurality of compounds.

An electron buffer layer, a hole blocking layer, an electron transport layer, an electron injection layer, or a combination thereof can be used between the light-emitting layer and the cathode. The electron buffer layer may be multilayers in order to control the injection of the electron and improve the interfacial properties between the light-emitting layer and the electron injection layer, wherein each of the multilayers may use two compounds simultaneously. The hole blocking layer or the electron transport layer may also be multilayers, wherein each of the multilayers may use a plurality of compounds.

The dopant comprised in the organic electroluminescent device of the present disclosure may be at least one phosphorescent or fluorescent dopant, and is preferably at least one phosphorescent dopant. The phosphorescent dopant material applied to the organic electroluminescent device of the present disclosure is not particularly limited, but may be preferably selected from metallated complex compounds of iridium (Ir), osmium (Os), copper (Cu), and platinum (Pt), more preferably selected from ortho-metallated complex compounds of iridium (Ir), osmium (Os), copper (Cu), and platinum (Pt), and even more preferably ortho-metallated iridium complex compounds.

The dopant comprised in the organic electroluminescent device of the present disclosure may comprise a compound represented by the following formula 101, but is not limited thereto.

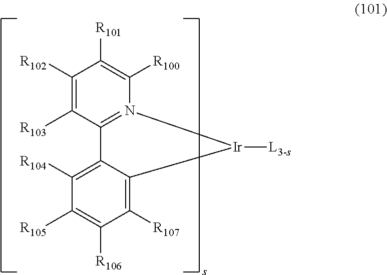

In formula 101, L is selected from the following structures 1 and 2:

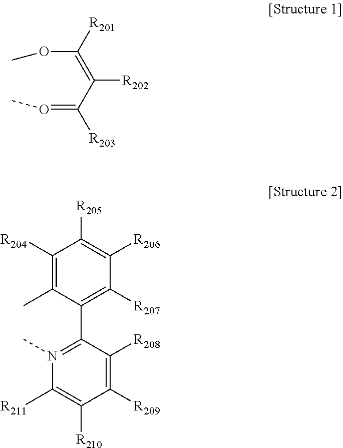

R₁₀₀ to R₁₀₃, each independently, represent hydrogen, deuterium, a halogen, a (C1-C30)alkyl unsubstituted or substituted with a halogen(s), a substituted or unsubstituted (C3-C30)cycloalkyl, a substituted or unsubstituted (C6-C30)aryl, a cyano, a substituted or unsubstituted (3- to 30-membered)heteroaryl, or a substituted or unsubstituted (C1-C30)alkoxy; or may be linked to an adjacent substituent to form a ring(s), e.g., a substituted or unsubstituted, quinoline, benzofuropyridine, benzothienopyridine, indenopyridine, benzofuroquinoline, benzothienoquinoline, or indenoquinoline ring, together with pyridine;

R₁₄ to R₁₀₇, each independently, represent hydrogen, deuterium, a halogen, a (C1-C30)alkyl unsubstituted or substituted with a halogen(s), a substituted or unsubstituted (C3-C30)cycloalkyl, a substituted or unsubstituted (C6-C30)aryl, a substituted or unsubstituted (3- to 30-membered)heteroaryl, a cyano, or a substituted or unsubstituted (C1-C30)alkoxy; or may be linked to an adjacent substituent to form a ring(s), e.g., a substituted or unsubstituted, naphthyl, fluorene, dibenzothiophene, dibenzofuran, indenopyridine, benzofuropyridine, or benzothienopyridine ring, together with benzene;

R₂₀₁ to R₂₁₁, each independently, represent hydrogen, deuterium, a halogen, a (C1-C30)alkyl unsubstituted or substituted with a halogen(s), a substituted or unsubstituted (C3-C30)cycloalkyl, or a substituted or unsubstituted (C6-C30)aryl; or may be linked to an adjacent substituent to form a ring(s); and

s represents an integer of 1 to 3.

The specific examples of the dopant compound are as follows, but are not limited thereto.

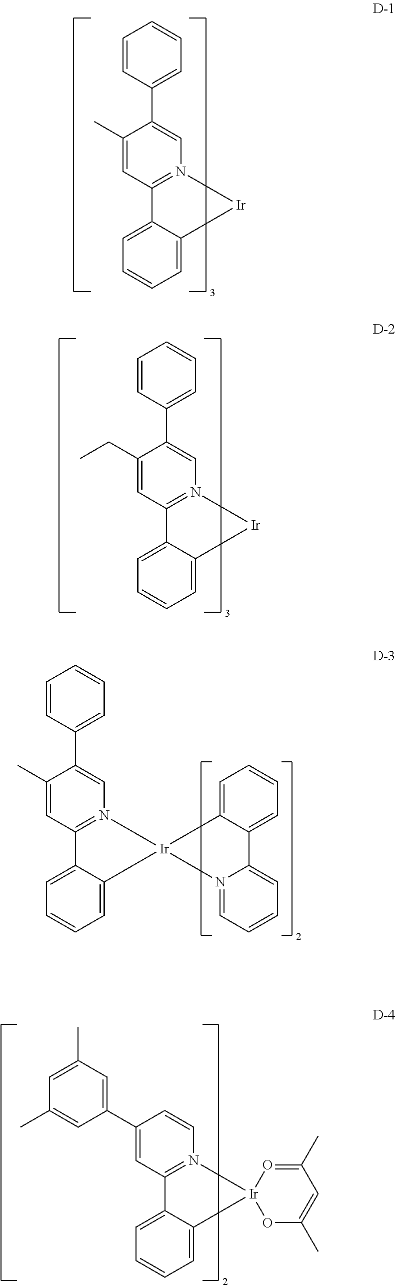

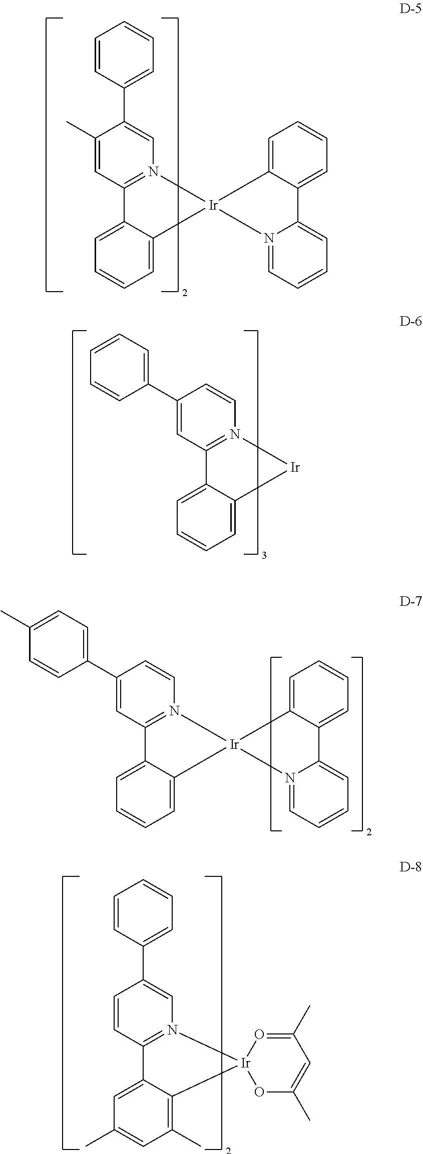

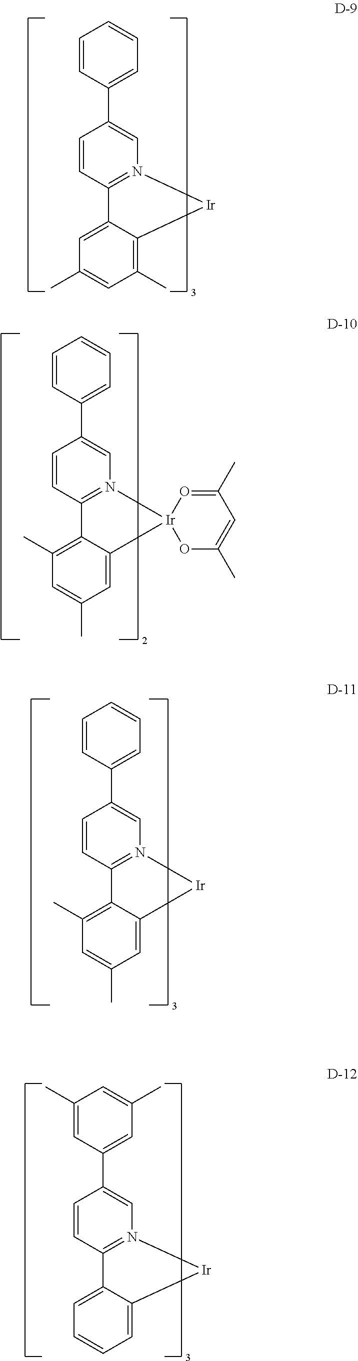

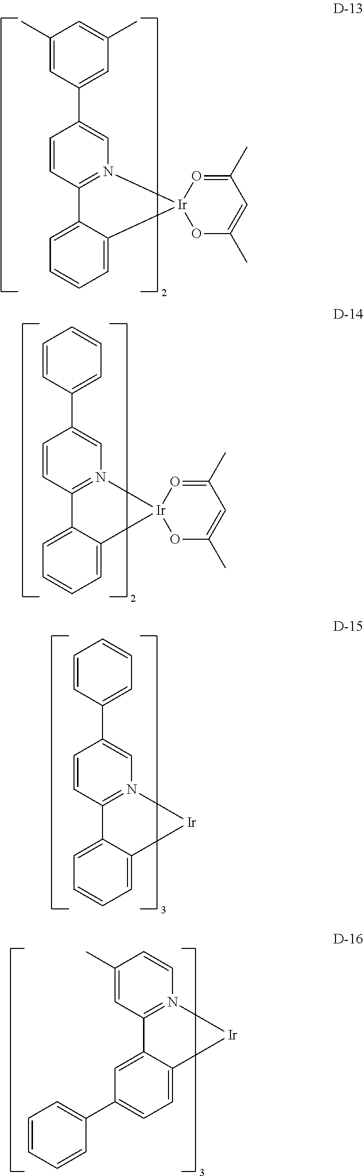

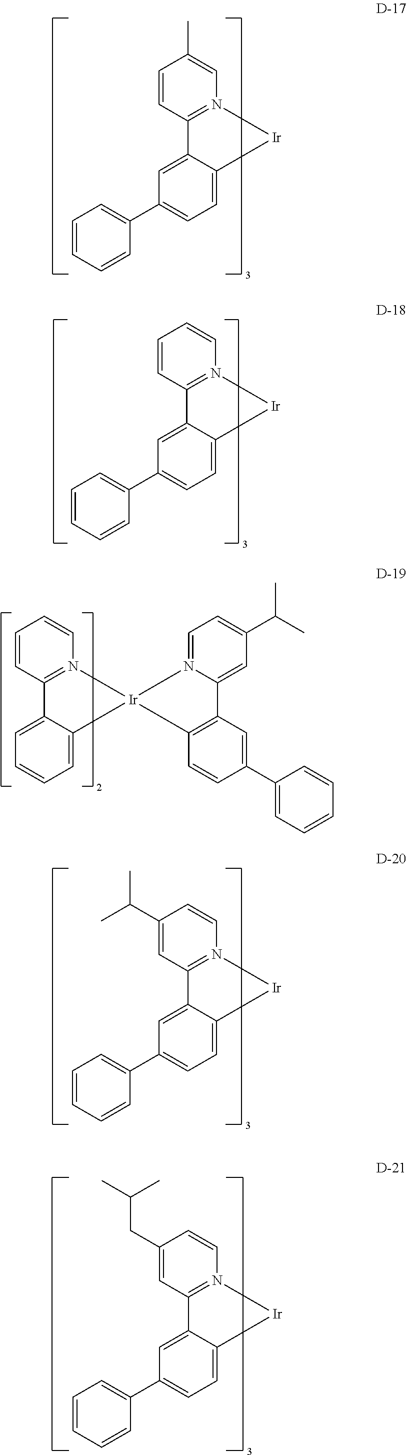

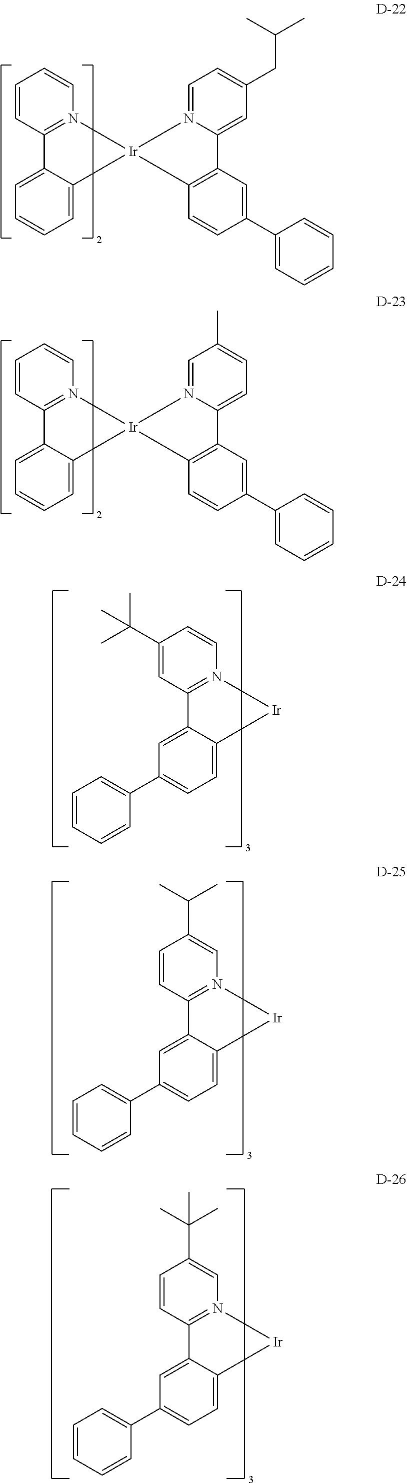

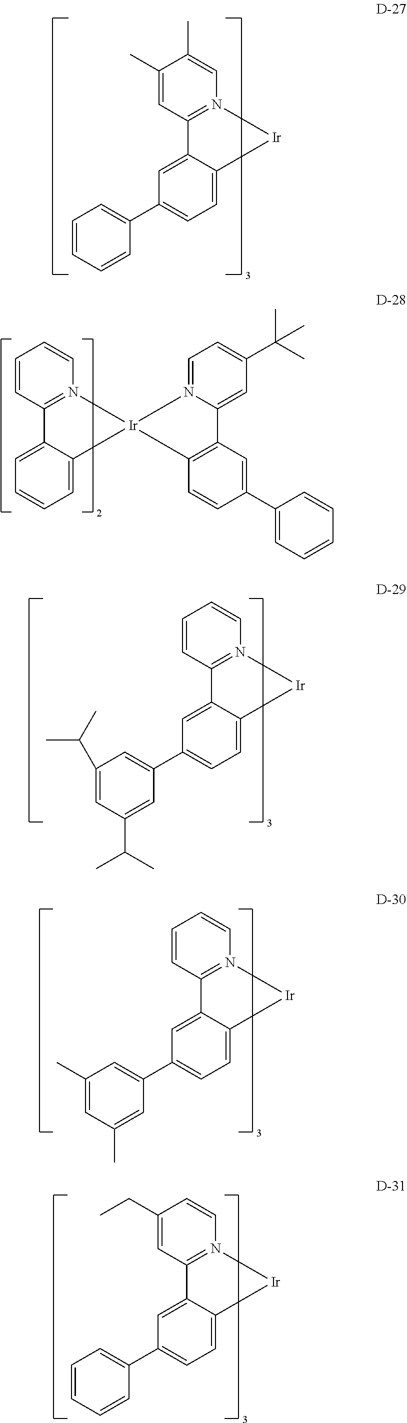

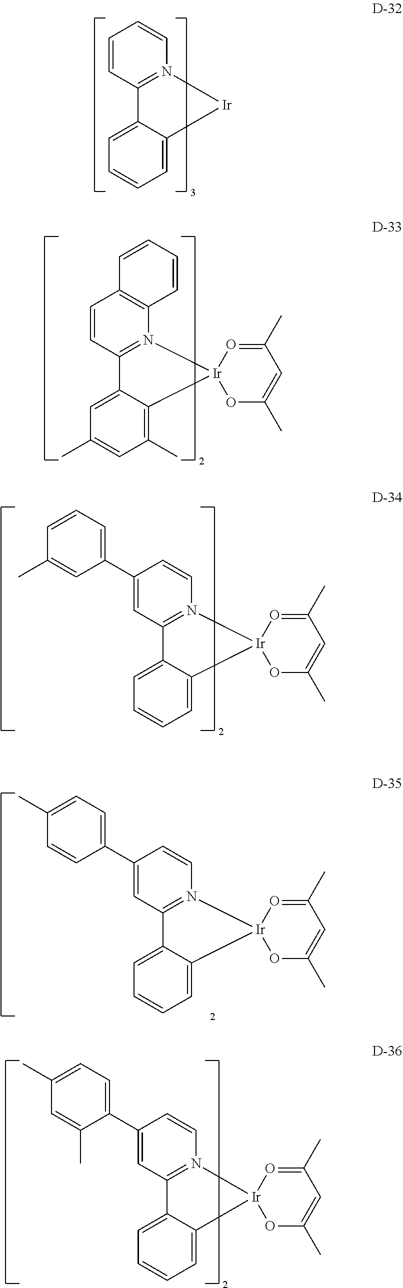

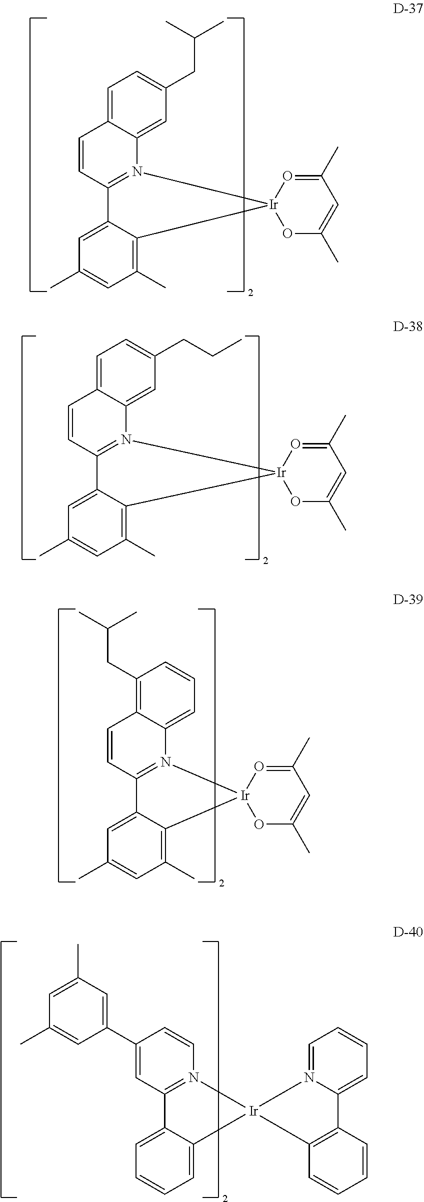

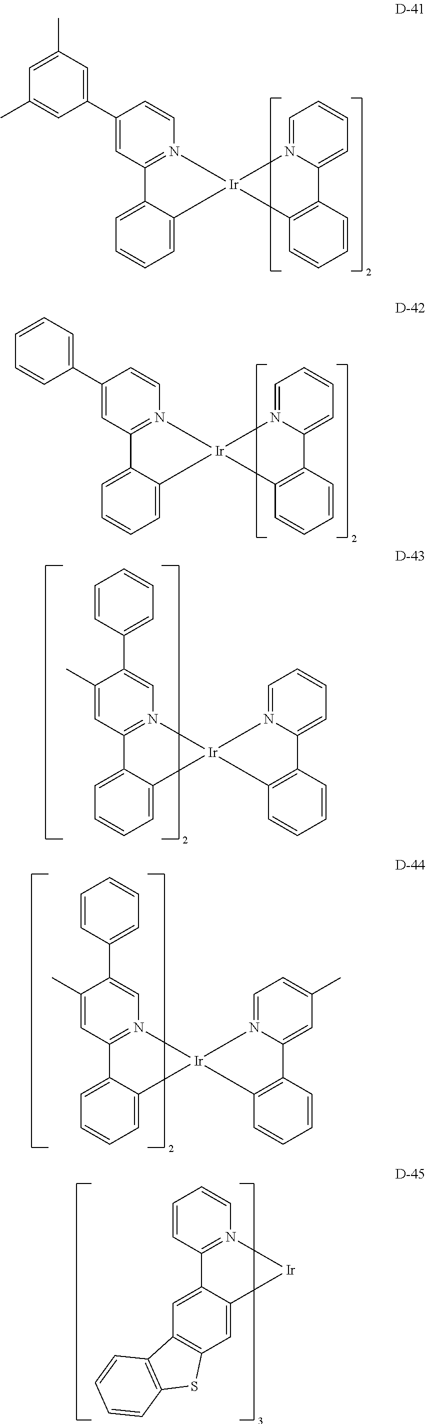

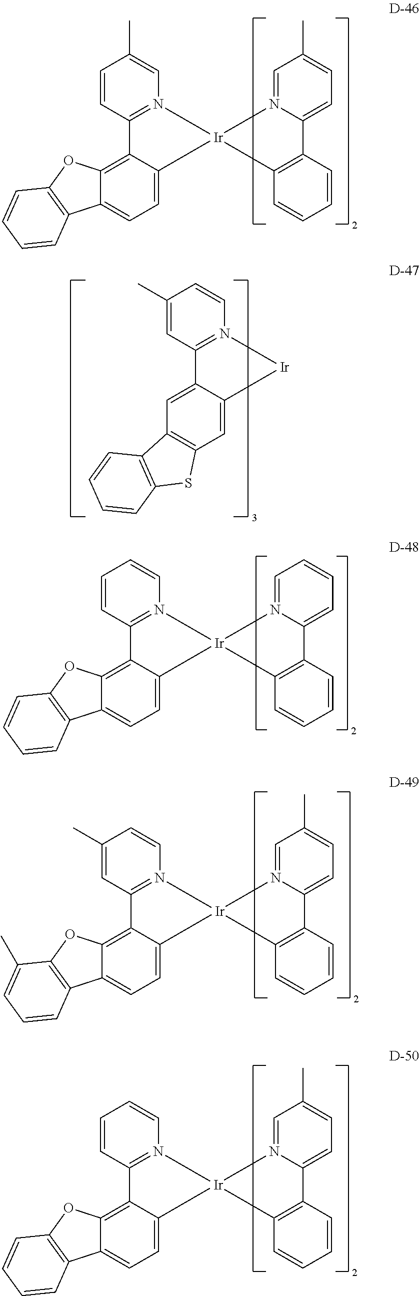

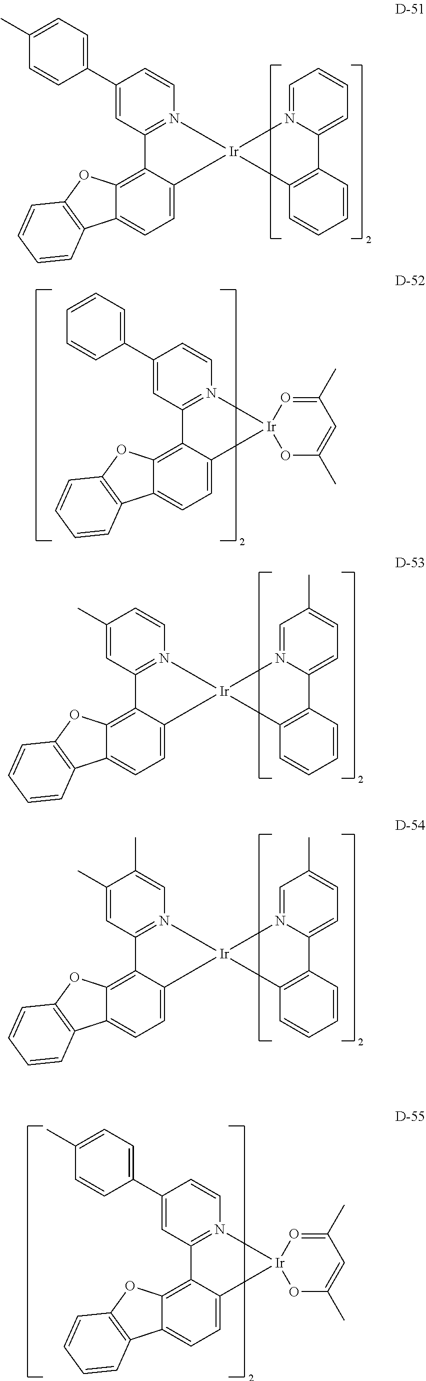

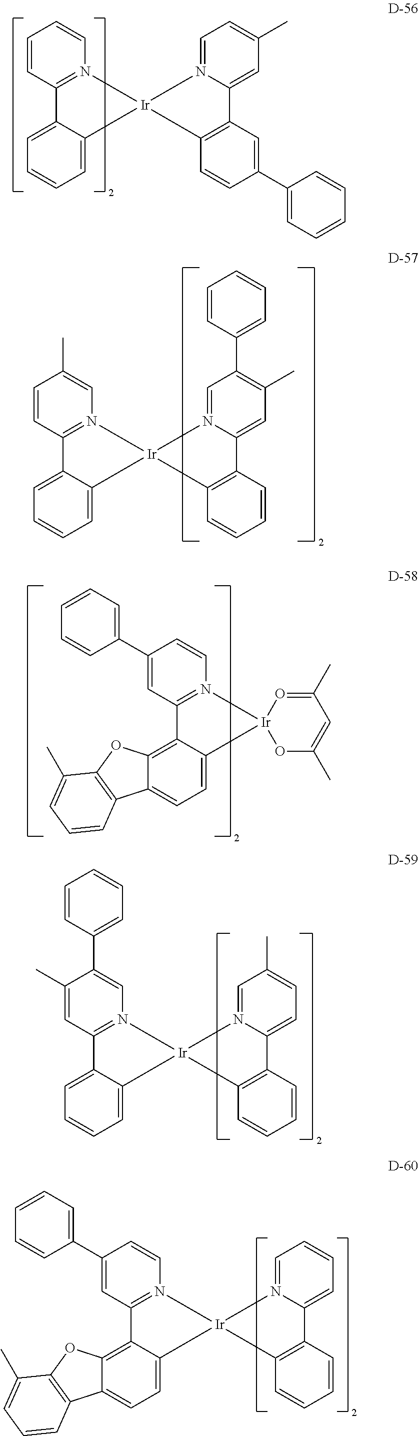

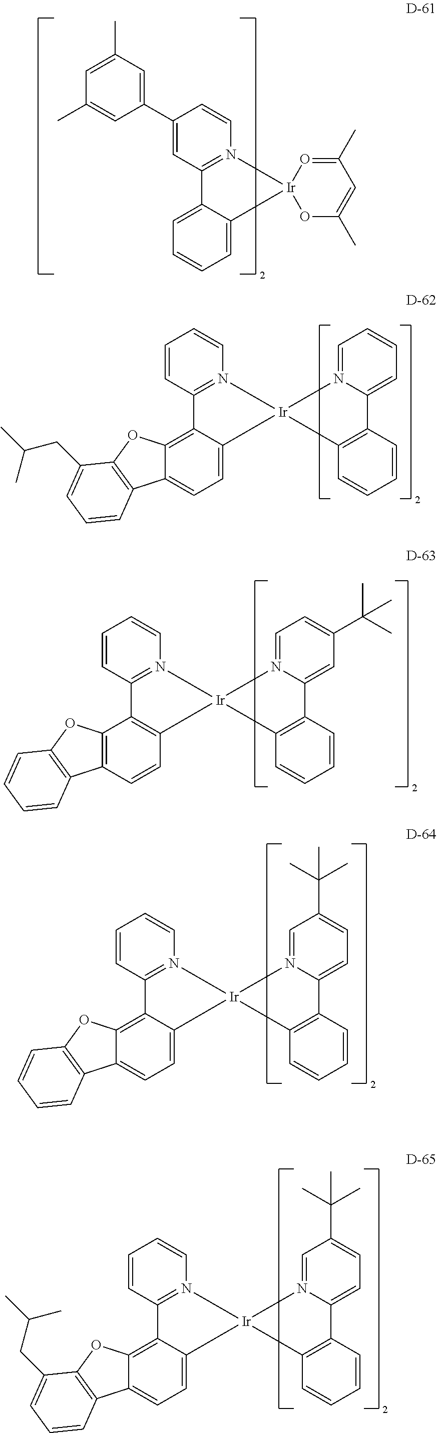

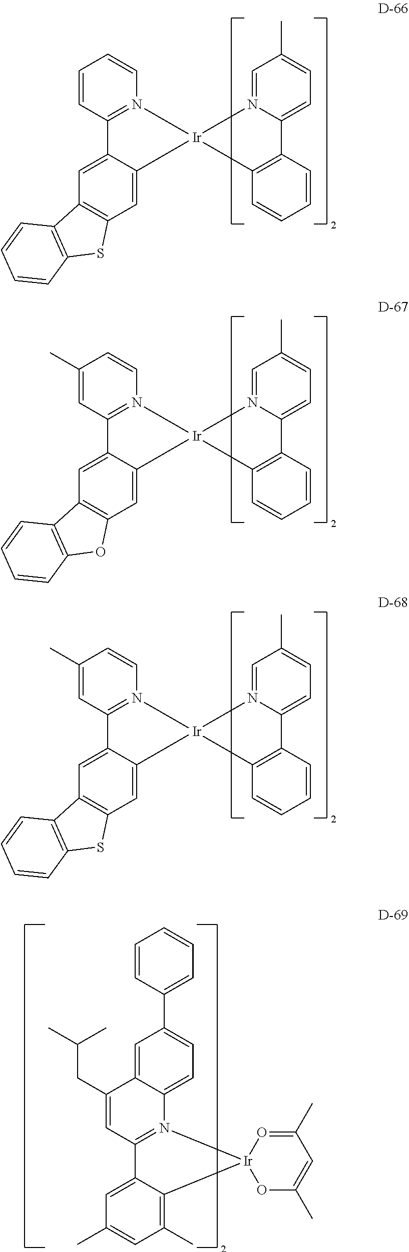

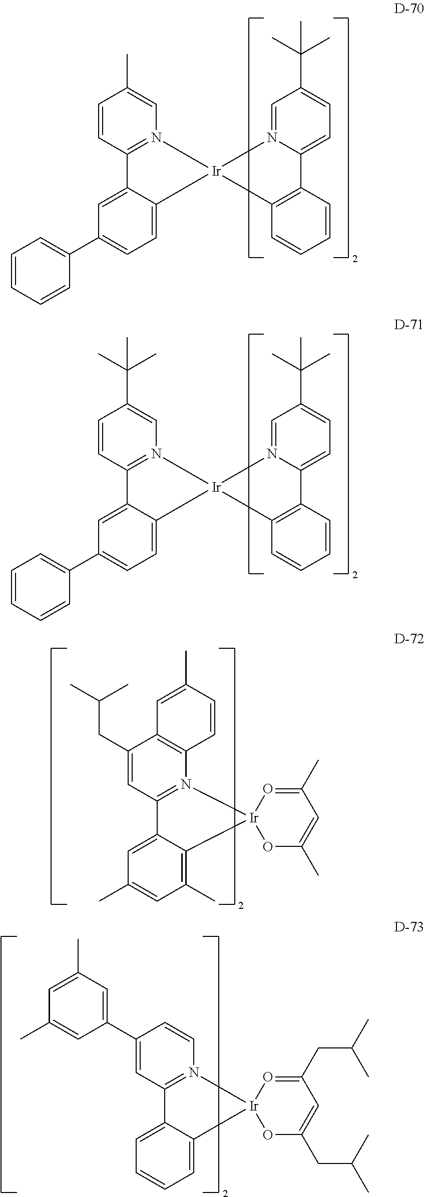

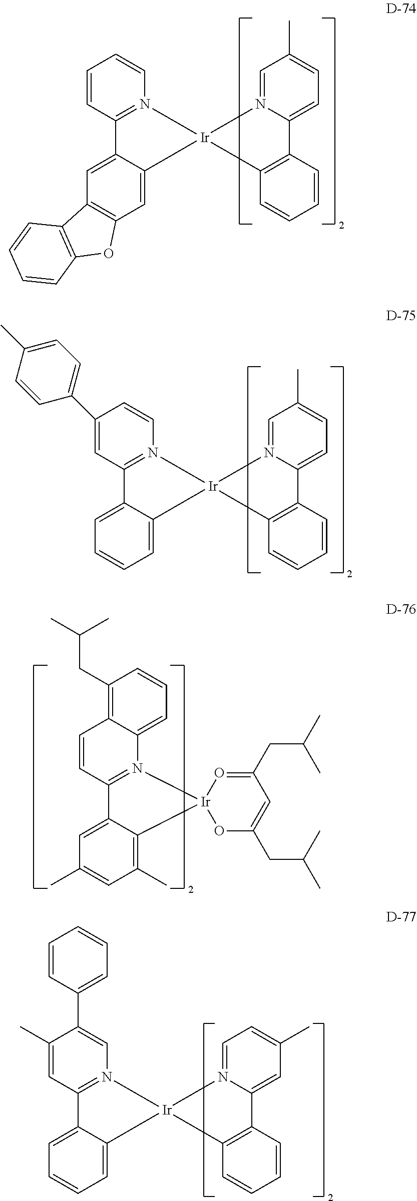

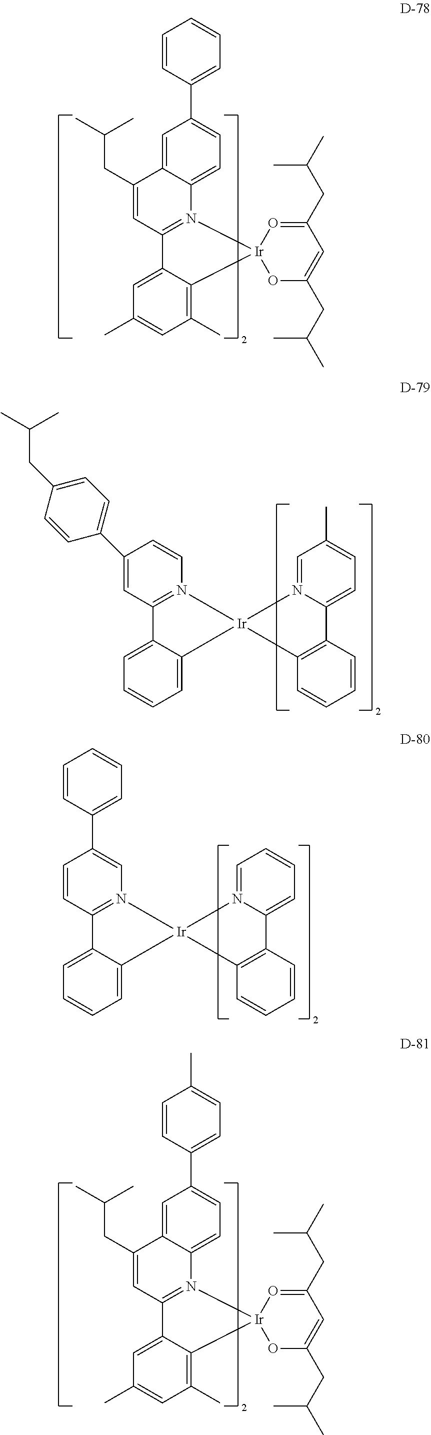

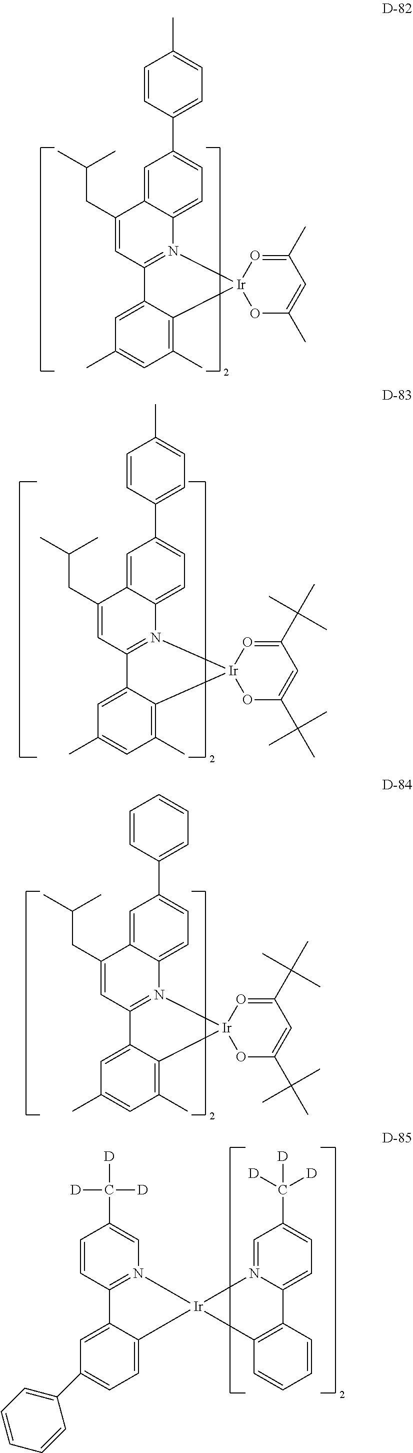

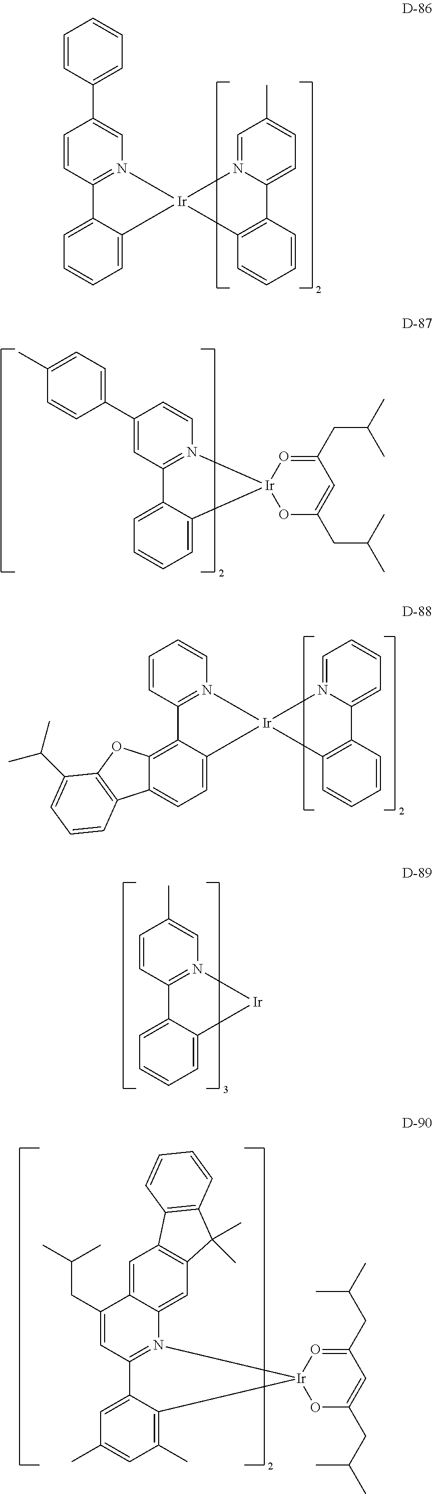

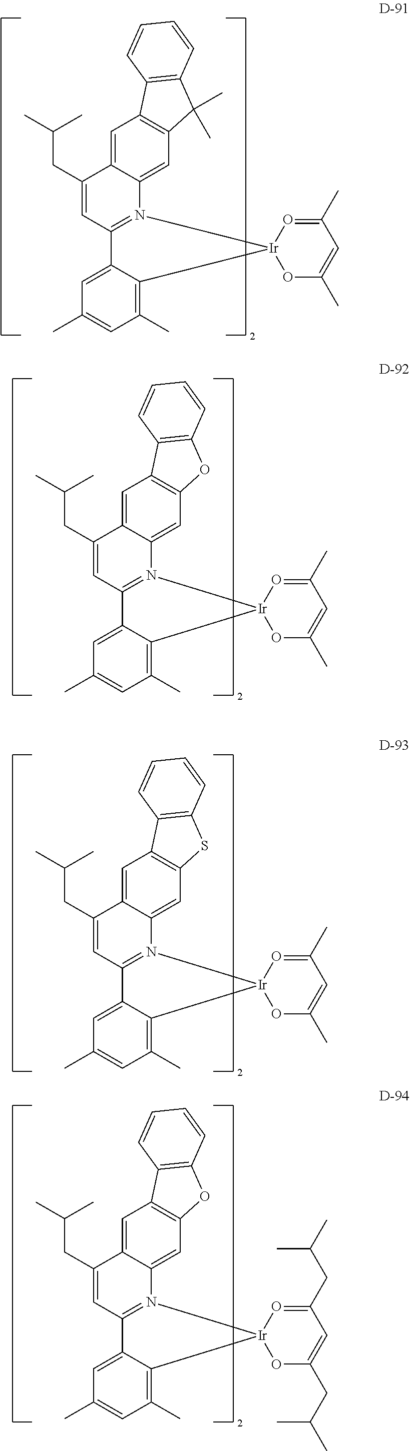

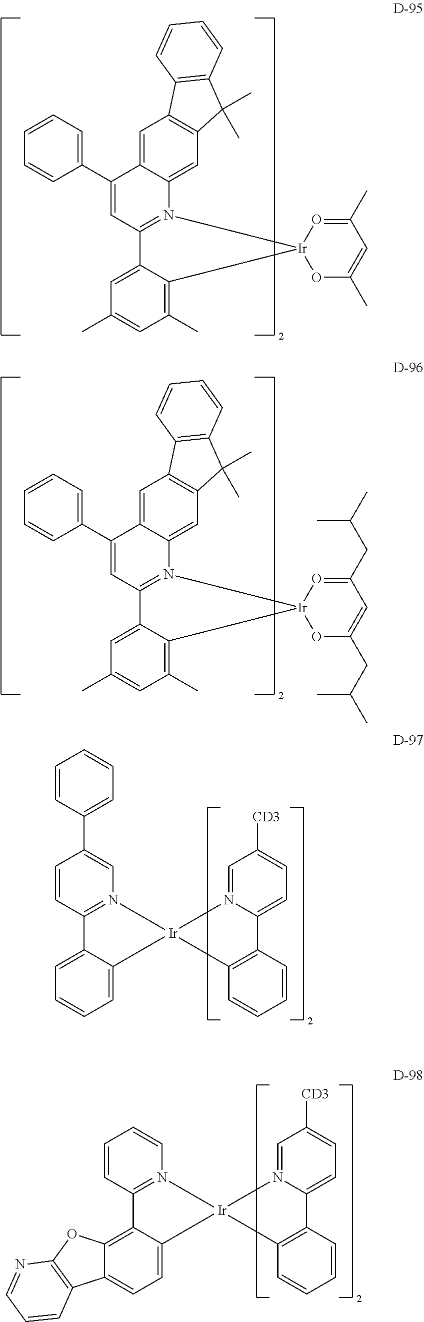

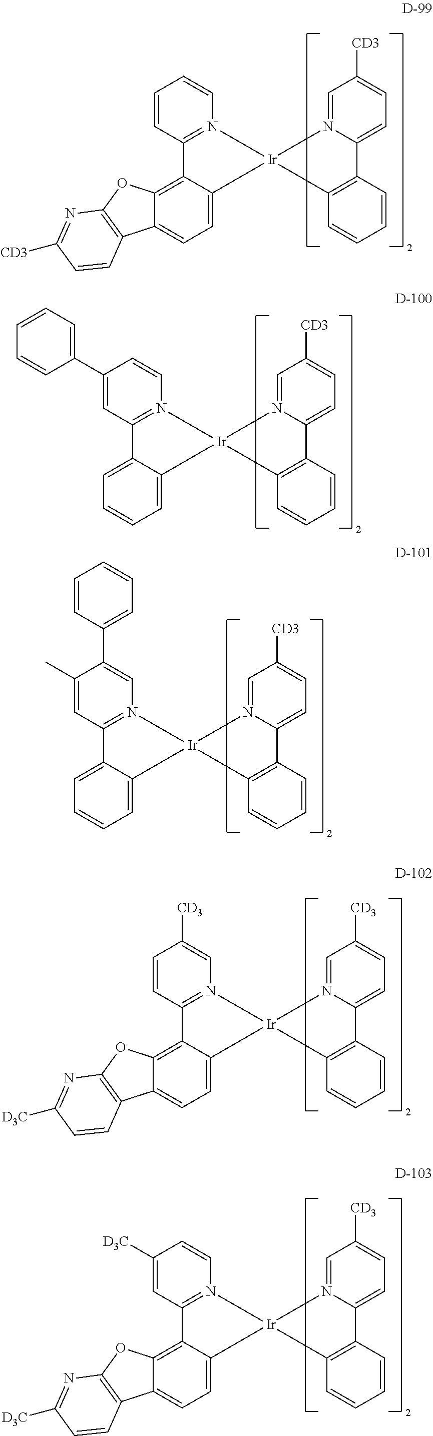

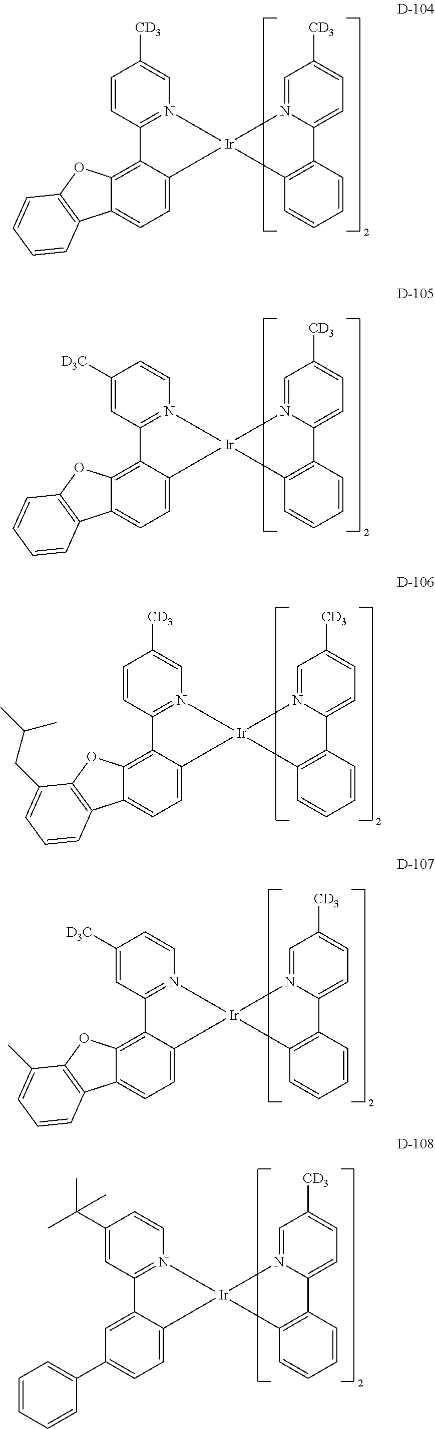

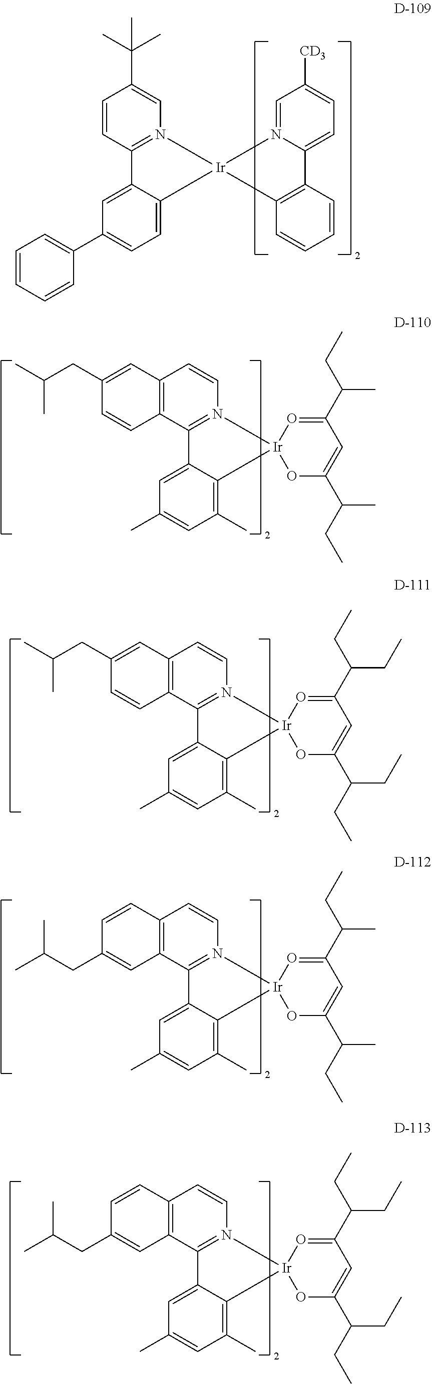

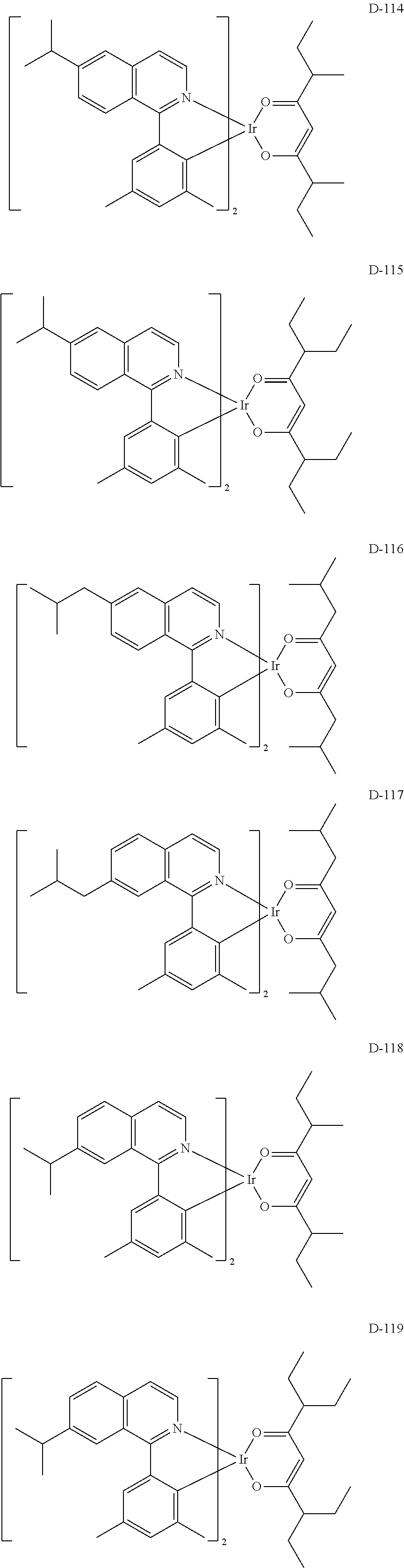

In order to form each layer of the organic electroluminescent device of the present disclosure, dry film-forming methods such as vacuum evaporation, sputtering, plasma, ion plating methods, etc., or wet film-forming methods such as ink jet printing, nozzle printing, slot coating, spin coating, dip coating, flow coating methods, etc., can be used.

When using a wet film-forming method, a thin film can be formed by dissolving or diffusing materials forming each layer into any suitable solvent such as ethanol, chloroform, tetrahydrofuran, dioxane, etc. The solvent can be any solvent where the materials forming each layer can be dissolved or diffused, and where there are no problems in film-formation capability.

The first and the second host compounds of the present disclosure may be film-formed by the above-listed methods, commonly by a co-evaporation process or a mixture-evaporation process. The co-evaporation is a mixed deposition method in which two or more materials are placed in a respective individual crucible source and a current is applied to both cells at the same time to evaporate the materials. The mixture-evaporation is a mixed deposition method in which two or more materials are mixed in one crucible source before evaporating them, and a current is applied to the cell to evaporate the materials. Further, if the first and the second host compounds are present in the same layer or different layers in an organic electroluminescent device, the two host compounds may individually form films. For example, the second host compound may be deposited after depositing the first host compound.

The present disclosure may provide a display device by using the plurality of host materials including the compound represented by formula 1 and the compound represented by formula 2. That is, by using the plurality of host materials of the present disclosure, it is possible to manufacture a display system or a lighting system. Specifically, by using the plurality of host materials of the present disclosure, a display system, for example, for white organic light emitting devices, smart phones, tablets, notebooks, PCs, TVs, or cars; or a lighting system, for example an outdoor or indoor lighting system, can be produced.

Hereinafter, the preparation method of the compound represented by formula 1 and the compound represented by formula 2 of the present disclosure and the properties thereof, and the properties of an organic electroluminescent device comprising the plurality of host materials of the present disclosure will be explained in detail with reference to the representative compounds of the present disclosure. However, the present disclosure is not limited by the following examples.

### Synthetic Example 1: Preparation of Compound H-1-52

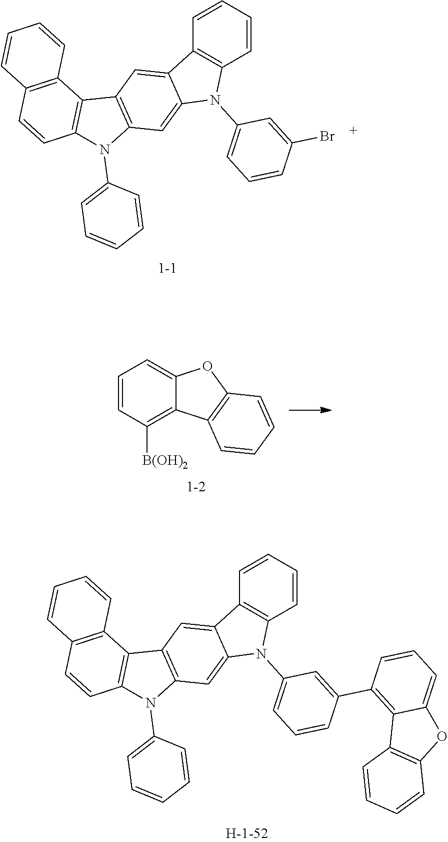 [SMILES: Brc1cccc(-n2c3ccccc3c3cc4c5c6ccccc6ccc5n(-c5ccccc5)c4cc32)c1 ; OB(O)c1cccc2oc3ccccc3c12 ; c1ccc(-n2c3cc4c(cc3c3c5ccccc5ccc32)c2ccccc2n4-c2cccc(-c3cccc4oc5ccccc5c34)c2)cc1]

Compound 1-1 (7 g, 13 mmol), Compound 1-2 (3 g, 14.3 mmol), K₂CO₃ (5.4 g, 39 mmol), and Pd(PPh₃)₄ (0.75 g, 0.65 mmol) were dissolved in 30 mL of H₂O, 60 mL of toluene, and 30 mL of EtOH, and then the mixture was refluxed at 120° C. for 3 hours. After completion of the reaction, the organic layer was extracted with ethyl acetate, and the residual moisture was removed by using magnesium sulfate. The residue was dried, and separated by column chromatography to obtain compound H-1-52 (5.7 g, yield: 70%).

| | MW | M.P. |
|---|---|---|
| H-1-52 | 642.73 | 154° C. |

### Synthetic Example 2: Preparation of Compound H-1-55

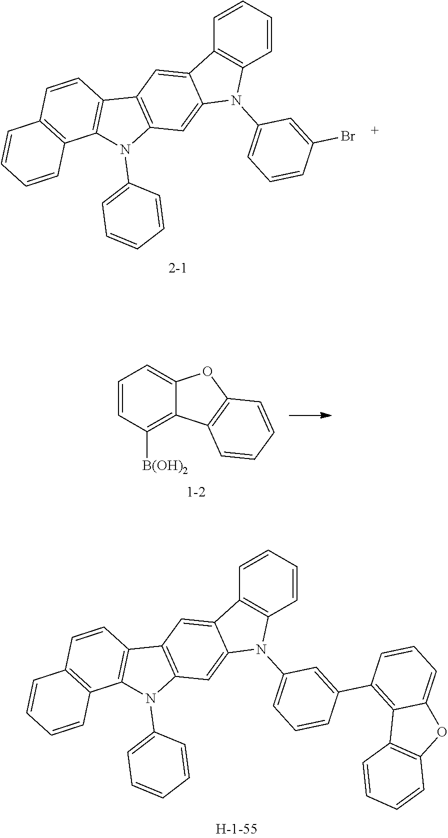 [SMILES: Brc1cccc(-n2c3ccccc3c3cc4c5ccc6ccccc6c5n(-c5ccccc5)c4cc32)c1 ; OB(O)c1cccc2oc3ccccc3c12 ; c1ccc(-n2c3cc4c(cc3c3ccc5ccccc5c32)c2ccccc2n4-c2cccc(-c3cccc4oc5ccccc5c34)c2)cc1]

Compound 2-1 (5.7 g, 10.6 mmol), compound 1-2 (2.5 g, 11.7 mmol), K₂CO₃ (4.4 g, 31.8 mmol), and Pd(PPh₃)₄ (0.61 g, 0.653 mmol) were dissolved in 30 mL of H₂O, 60 mL of toluene, and 30 mL of EtOH, and then the mixture was refluxed at 120° C. for 3 hours. After completion of the reaction, the organic layer was extracted with ethyl acetate, and the residual moisture was removed by using magnesium sulfate. The residue was dried, and separated by column chromatography to obtain compound H-1-55 (1.2 g, yield: 18%).

| | MW | M.P. |
|---|---|---|
| H-1-55 | 642.73 | 239° C. |

### Synthetic Example 3: Preparation of Compound H-1-46

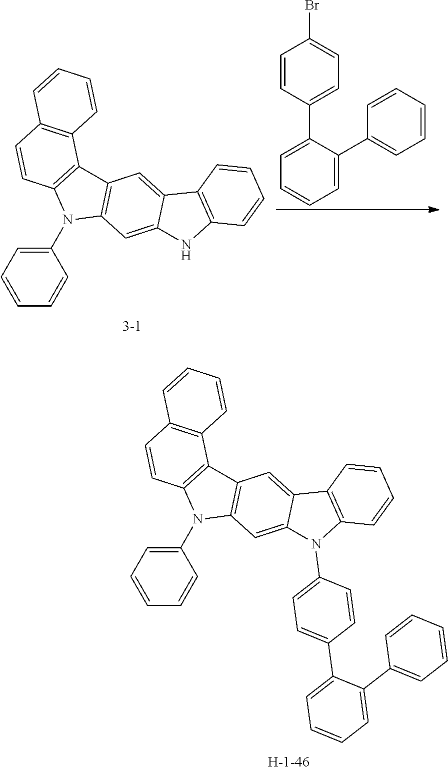

70 mL of xylene was added to compound 3-1 (5 g, 13.07 mmol), 4-bromo-1,1′:2′,1″-terphenyl (4 g, 13.07 mmol), Pd₂(dba)₃ (0.6 g, 0.653 mmol), NaOt-Bu (3.8 g, 39.21 mmol), and 2-dicyclohexylphosphino-2′,6′-dimethoxybiphenyl (s-phos) (0.5 g, 1.307 mmol), and then the mixture was stirred for one day. After completion of the reaction, the mixture was cooled to room temperature, and extracted with distilled water and MeOH. The resulting product was separated by column chromatography using methylene chloride (MC)/Hex to obtain compound H-1-46 (6.3 g, yield: 78%).

| | MW | M.P. |
|---|---|---|
| H-1-46 | 610.7 | 288° C. |

### Synthetic Example 4: Preparation of Compound H-1-47

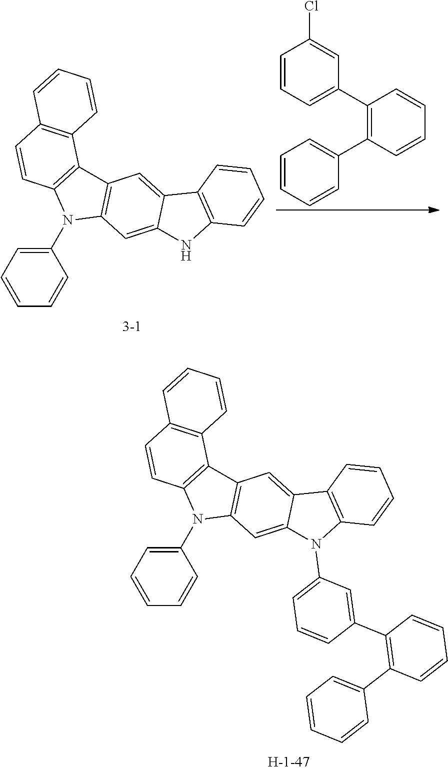

100 mL of toluene was added to compound 3-1 (7.6 g, 18.88 mmol), 3-chloro-1,1′:2′,1″-terphenyl (5 g, 18.88 mmol), Pd₂(dba)₃ (0.86 g, 0.940 mmol), NaOt-Bu (4.5 g, 47.22 mmol), and P(t-Bu)₃ (0.38 g, 1.888 mmol), and then the mixture was stirred for one day. After completion of the reaction, the mixture was cooled to room temperature, and extracted with distilled water and MeOH. The resulting product was separated by column chromatography using MC/Hex to obtain compound H-1-47 (0.7 g, yield: 6.2%).

| | MW | M.P. |
|---|---|---|
| H-1-47 | 610.7 | 194.6° C. |

### Synthetic Example 5: Preparation of Compound H-1-115

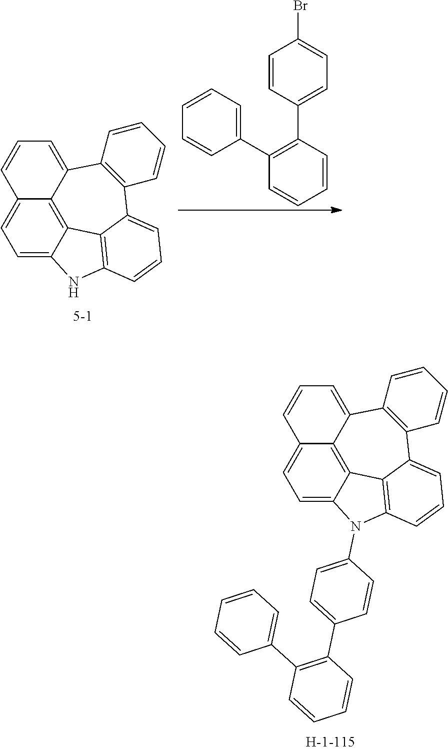

86 mL of o-xylene was introduced into compound 5-1 (5 g, 17.16 mmol), 4-bromo-1,1′:2′,1″-terphenyl (5.3 g, 17.16 mmol), Pd₂(dba)₃ (0.8 g, 0.858 mmol), s-phos (0.7 g, 1.716 mmol), and NaOt-Bu (5 g, 51.48 mmol), and then the mixture was stirred under reflux for 2 hours. After completion of the reaction, the mixture was cooled to room temperature, and the resulting solid was filtered under reduced pressure. The solid was dissolved in CHCl₃, and separated by column chromatography using MC/Hex to obtain compound H-1-115 (2.4 g, yield: 26%).

| | MW | M.P. |
|---|---|---|
| H-1-115 | 519.6 | 228.7° C. |

### Synthetic Example 6: Preparation of Compound H-1-142

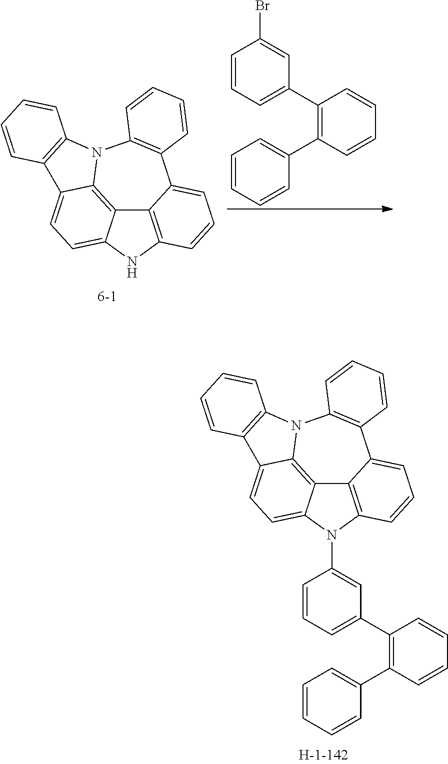

Compound 6-1 (40.0 g, 121.0 mmol), 3-bromo-1,1′:2′,1″-terphenyl (41.1 g, 133.1 mmol), Pd₂(dba)₃ (5.5 g, 6.1 mmol), s-phos (5.0 g, 12.1 mmol), and NaOt-Bu (34.8 g, 363.0 mmol) were dissolved in 600 mL of o-xylene, and then the mixture was refluxed at 170° C. for 5 hours. After completion of the reaction, the organic layer was extracted with ethyl acetate, and the residual moisture was removed by using magnesium sulfate. The residue was dried, and separated by column chromatography to obtain compound H-1-142 (28.5 g, yield: 42%).

| | MW | M.P. |
|---|---|---|
| H-1-142 | 558.7 | 248° C. |

### Synthetic Example 7: Preparation of Compound H-1-143

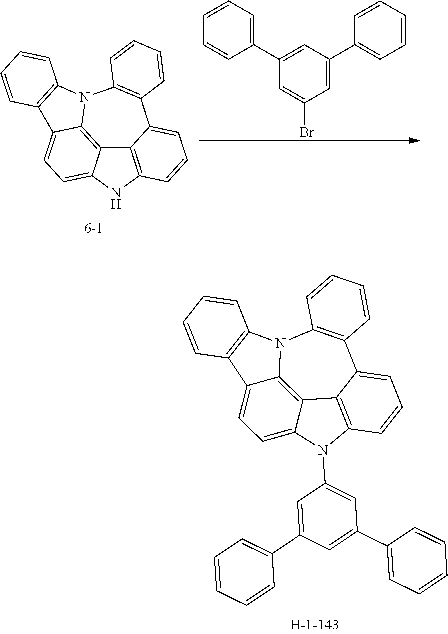

Compound 6-1 (4.4 g, 13.5 mmol), 5′-bromo-1,1′,3′,1″-terphenyl (5 g, 16.2 mmol), CuI (1.3 g, 6.75 mmol), ethylene diamine (1.8 mL, 27 mmol), and K₃PO₄ (8.6 g, 40.5 mmol) were dissolved in 67.5 mL of toluene, and then the mixture was refluxed at 120° C. for 2 hours. After completion of the reaction, the organic layer was extracted with ethyl acetate, and the residual moisture was removed by using magnesium sulfate. The residue was dried, and separated by column chromatography to obtain compound H-1-143 (6 g, yield: 79.6%).

| | MW | M.P. |
|---|---|---|
| H-1-143 | 558.7 | 276.6° C. |

### Synthetic Example 8: Preparation of Compound C-5

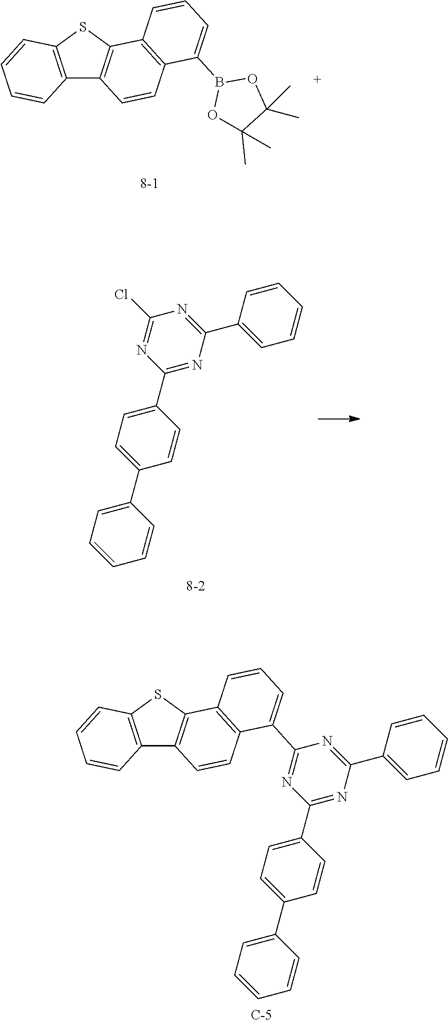

5.0 mL of EtOH, 40 mL of toluene, and 11 mL of distilled water were added to compound 8-1 (4.0 g, 11.1 mmol), compound 8-2 (4.6 g, 13.3 mmol), Pd(PPh₃)₄ (0.6 g, 0.56 mmol), and K₂CO₃ (3.1 g, 22.2 mmol), and then the mixture was stirred under reflux for 6 hours. After completion of the reaction, the mixture was cooled to room temperature, and stirred at ambient temperature. MeOH was added to the mixture to obtain a solid. The resulting solid was filtered under reduced pressure, and separated by column chromatography using MC/Hex to obtain compound C-5 (4.9 g, yield: 81%).

| | MW | M.P. |
|---|---|---|
| C-5 | 541.7 | 280° C. |

### Synthetic Example 9: Preparation of Compound C-146

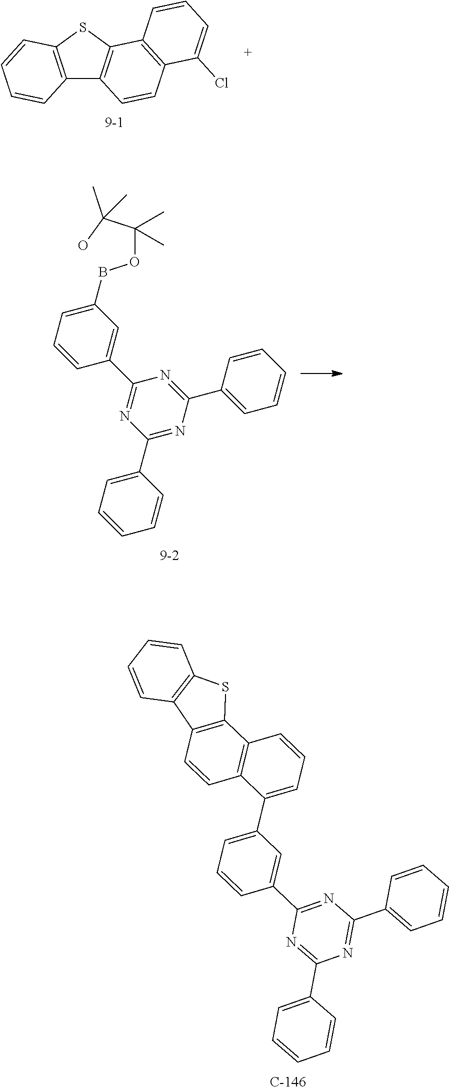

80 mL of o-xylene was added to compound 9-1 (4.0 g, 14.9 mmol), compound 9-2 (7.1 g, 16.4 mmol), Pd₂(dba)₃ (0.7 g, 0.8 mmol), s-phos (0.6 g, 1.5 mmol), and NaOtBu (3.5 g, 37.3 mmol), and then the mixture was stirred under reflux for 6 hours. After completion of the reaction, the mixture was cooled to room temperature, and stirred at ambient temperature. MeOH was added to the mixture to obtain a solid. The resulting solid was filtered under reduced pressure, and separated by column chromatography using MC/Hex to obtain compound C-146 (3.6 g, yield: 45%).

| | MW | M.P. |
|---|---|---|
| C-146 | 541.7 | 261° C. |

### Synthetic Example 10: Preparation of Compound C-160

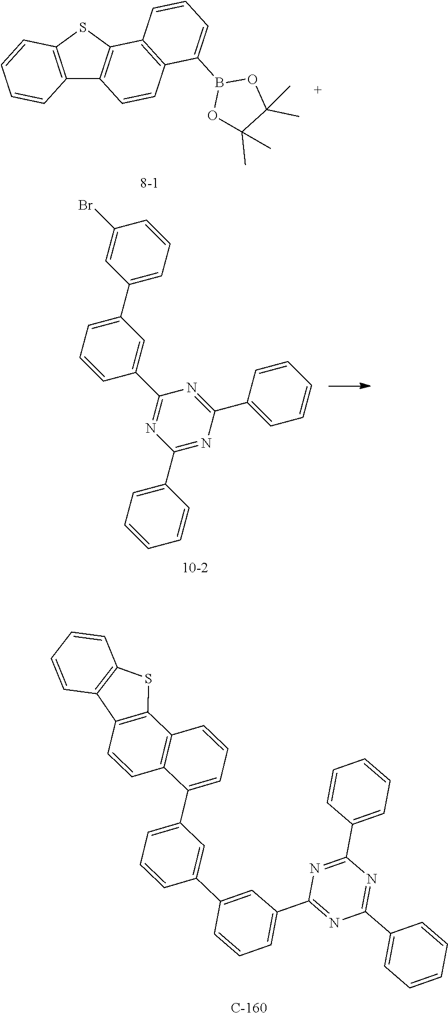 [SMILES: CC1(C)OB(c2cccc3c2ccc2c4ccccc4sc32)OC1(C)C ; Brc1cccc(-c2cccc(-c3nc(-c4ccccc4)nc(-c4ccccc4)n3)c2)c1 ; c1ccc(-c2nc(-c3ccccc3)nc(-c3cccc(-c4cccc(-c5cccc6c5ccc5c7ccccc7sc65)c4)c3)n2)cc1]

55 mL of toluene, 14 mL of ethanol, and 14 mL of distilled water were added to compound 8-1 (4.5 g, 12.49 mmol), compound 10-2 (6.6 g, 14.20 mmol), tetrakis(triphenylphosphine)palladium (0.4 g, 0.34 mmol), and sodium carbonate (3.0 g, 28.38 mmol), and then the mixture was stirred at 130° C. for 4 hours. After completion of the reaction, the resulting solid was washed with distilled water and methanol, and then purified by column chromatography to obtain compound C-160 (3.9 g, yield: 51%).

| | MW | M.P. |
|---|---|---|
| C-160 | 617.7 | 268° C. |

### Synthetic Example 11: Preparation of Compound C-230

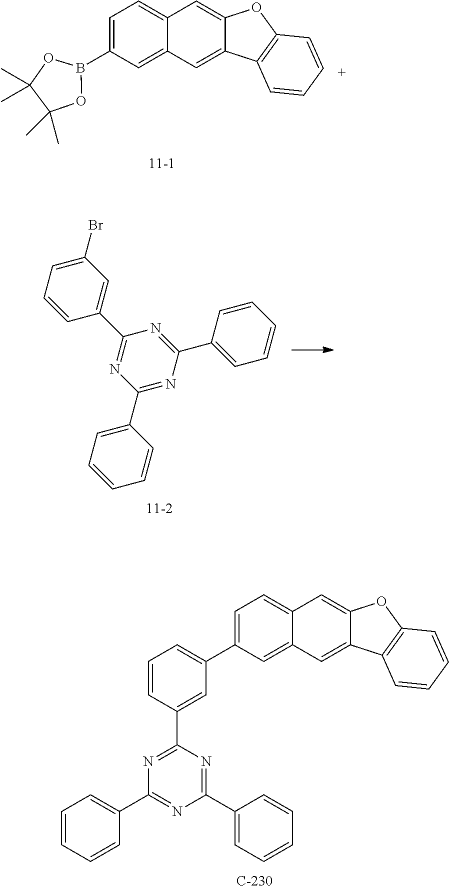 [SMILES: CC1(C)OB(c2ccc3cc4oc5ccccc5c4cc3c2)OC1(C)C ; Brc1cccc(-c2nc(-c3ccccc3)nc(-c3ccccc3)n2)c1 ; c1ccc(-c2nc(-c3ccccc3)nc(-c3cccc(-c4ccc5cc6oc7ccccc7c6cc5c4)c3)n2)cc1]

80 mL of toluene, 20 mL of ethanol, and 20 mL of water were added to compound 11-1 (4.5 g, 13.07 mmol), compound 11-2 (5 g, 13.07 mmol), tetrakis(triphenylphosphine)palladium (0.75 g, 0.653 mmol), and potassium carbonate (5.4 g, 39.22 mmol), and then the mixture was stirred under reflux for 2 hours. After completion of the reaction, the mixture was cooled to ambient temperature, and methanol was added dropwise thereto. The resulting solid was filtered, dissolved in dimethyl chloride, and separated by column chromatography to obtain compound C-230 (3.7 g, yield: 53%).

| | MW | M.P. |
|---|---|---|
| C-230 | 525.6 | 272° C. |

### Synthetic Example 12: Preparation of Compound C-167

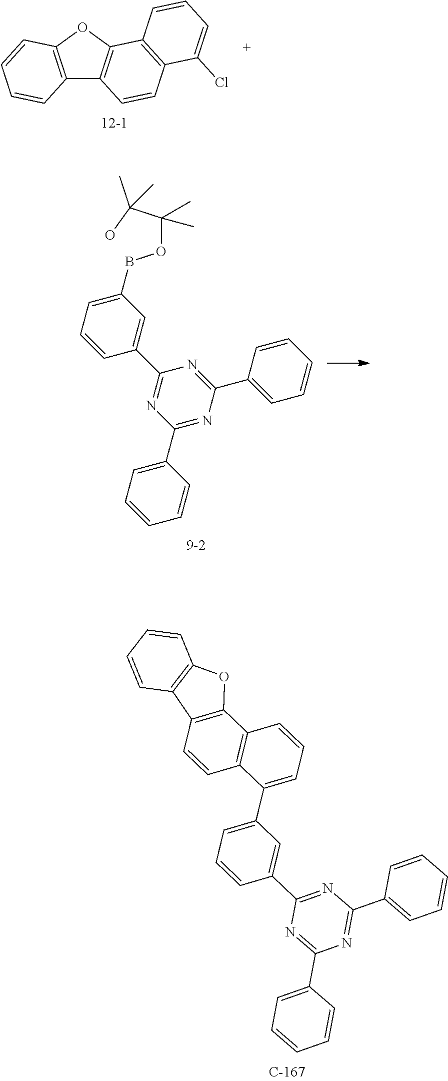

Compound 12-1 (5 g, 19.03 mmol), compound 9-2 (9.1 g, 20.94 mmol), tris (dibenzylideneacetone)dipalladium (0) (0.88 g, 0.97 mmol), dicyclohexylphosphino-2′,6′-dimethoxybiphenyl (0.79 g, 1.93 mmol), sodium tert-butoxide (4.63 g, 48.3 mmol), and 100 mL of xylene were added to a flask, and then the mixture was stirred under reflux for 4 hours. After completion of the reaction, the resulting product was extracted with ethyl acetate, and separated by column chromatography to obtain compound C-167 (5 g, yield: 50%).

| | MW | M.P. |
|---|---|---|
| C-167 | 525.6 | 252.6° C. |

### Synthetic Example 13: Preparation of Compound C-489

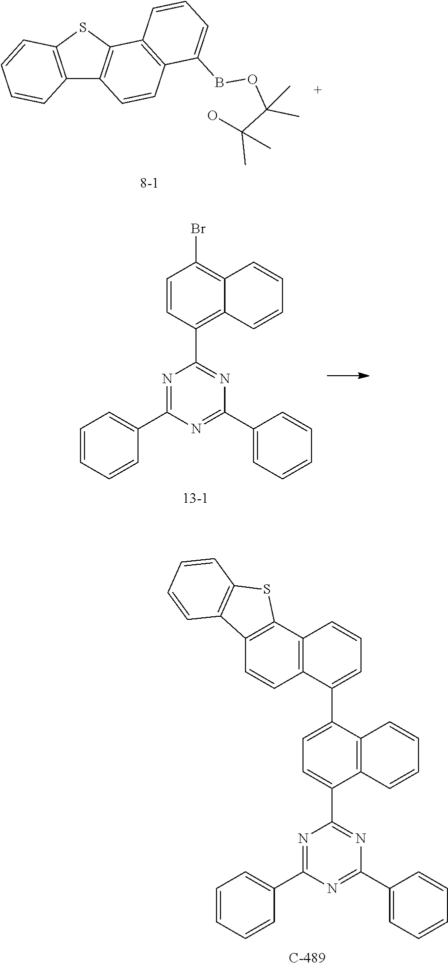

Compound 8-1 (5.0 g, 13.9 mmol), compound 13-1 (6.1 g, 13.9 mmol), tetrakis(triphenylphosphine)palladium (0.8 g, 0.7 mmol), potassium carbonate (3.9 g, 27.8 mmol), 30 mL of toluene, 10 mL of ethanol, and 14 mL of distilled water were added to a flask, and then the mixture was stirred at 130° C. for 5 hours. After completion of the reaction, the resulting solid was washed with distilled water and methanol, and then purified by column chromatography to obtain compound C-489 (3.6 g, yield: 44%).

| | MW | M.P. |
|---|---|---|
| C-489 | 591.7 | 282.5° C. |

### Synthetic Example 14: Preparation of Compound C-249

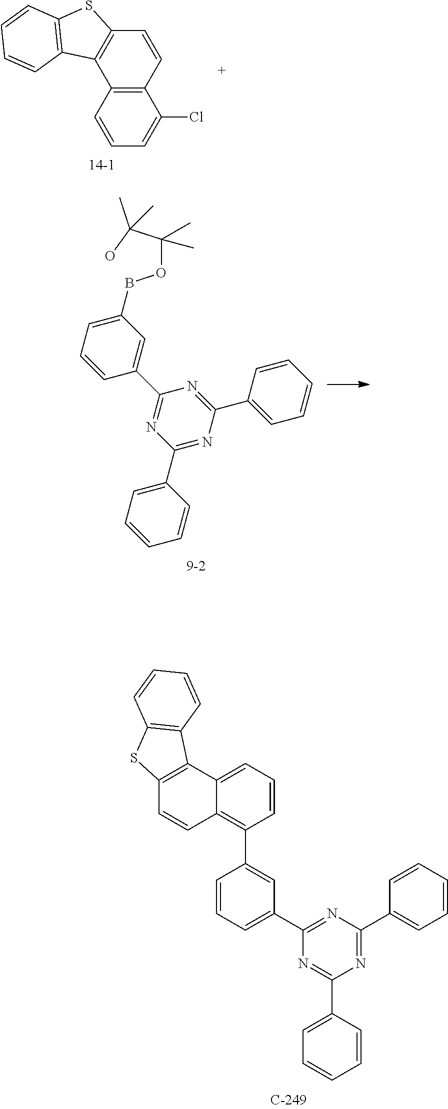

Compound 14-1 (4.0 g, 14.9 mmol), compound 9-2 (7.1 g, 16.4 mmol), tris(dibenzylideneacetone)dipalladium (0.7 g, 0.74 mmol), s-phos (0.6 g, 1.49 mmol), sodium tert-butoxide (3.5 g, 37.3 mmol), and 80 mL of o-xylene were added to a flask, and then the mixture was stirred at 165° C. for 5 hours. After completion of the reaction, the mixture was cooled to ambient temperature, and extracted with ethyl acetate. The extracted organic layer was dried with magnesium sulfate, and the solvent was removed by rotary evaporator. Thereafter, the residue was purified by column chromatography to obtain compound C-249 (4.2 g, yield: 81%).

| | MW | M.P. |
|---|---|---|
| C-249 | 541.7 | 283° C. |

### Synthetic Example 15: Preparation of Compound C-174

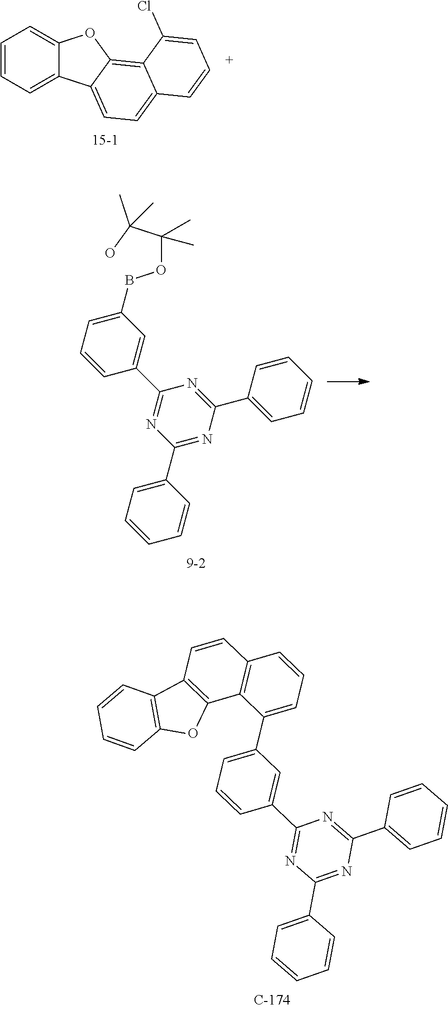

Compound 15-1 (6.0 g, 23.7 mmol), compound 9-2 (11.4 g, 26.1 mmol), tris(dibenzylideneacetone)dipalladium (1.1 g, 1.2 mmol), s-phos (0.98 g, 2.4 mmol), potassium phosphate (12.6 g, 59.3 mmol), and 120 mL of o-xylene were added to a flask, and then the mixture was stirred at 165° C. for 5 hours. After completion of the reaction, the mixture was cooled to ambient temperature, and extracted with ethyl acetate. The extracted organic layer was dried with magnesium sulfate, and the solvent was removed by rotary evaporator. Thereafter, the residue was purified by column chromatography to obtain compound C-174 (4.0 g, yield: 32%).

| | MW | M.P. |
|---|---|---|
| C-174 | 525.6 | 244° C. |

### Synthetic Example 16: Preparation of Compound C-520

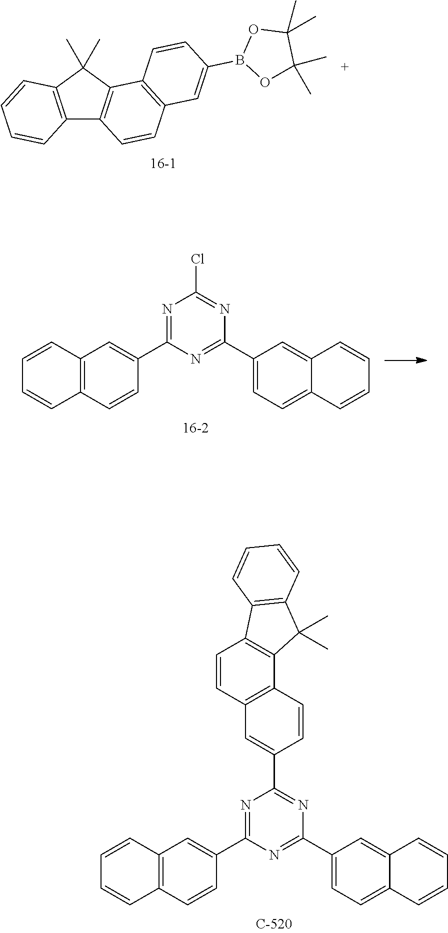 [SMILES: CC1(C)c2ccccc2-c2ccc3cc(B4OC(C)(C)C(C)(C)O4)ccc3c21 ; Clc1nc(-c2ccc3ccccc3c2)nc(-c2ccc3ccccc3c2)n1 ; CC1(C)c2ccccc2-c2ccc3cc(-c4nc(-c5ccc6ccccc6c5)nc(-c5ccc6ccccc6c5)n4)ccc3c21]

Compound 15-1 (4.23 g, 11.4 mmol), compound 15-2 (5.04 g, 13.7 mmol), tetrakis(triphenylphosphine)palladium (0.66 g, 0.57 mmol), potassium carbonate (3.15 g, 22.8 mmol), 35 mL of toluene, 7 mL of ethanol, and 11 mL of distilled water were added to a flask, and then the mixture was stirred at 130° C. for 15 hours. After completion of the reaction, the resulting solid was washed with distilled water and methanol, and purified by column chromatography to obtain compound C-520 (4.5 g, yield: 69%).

| | MW | M.P. |
|---|---|---|
| C-520 | 575.7 | 293° C. |

### Device Examples 1 to 4 and 8 to 15: Producing an OLED Deposited with the Plurality of Host Materials According to the Present Disclosure as a Host

OLEDs according to the present disclosure were produced. A transparent electrode indium tin oxide (ITO) thin film (10 Ω/sq) on a glass substrate for an OLED (GEOMATEC CO., LTD., Japan) was subjected to an ultrasonic washing with acetone, trichloroethylene, acetone, ethanol, and distilled water, sequentially, and then was stored in isopropyl alcohol. The ITO substrate was then mounted on a substrate holder of a vacuum vapor deposition apparatus. Compound HI-1 was introduced into a cell of the vacuum vapor deposition apparatus, and then the pressure in the chamber of the apparatus was controlled to 10⁻⁶ torr. Thereafter, an electric current was applied to the cell to evaporate the above-introduced material, thereby forming a first hole injection layer having a thickness of 80 nm on the ITO substrate. Next, compound HI-2 was introduced into another cell of the vacuum vapor deposition apparatus and was evaporated by applying an electric current to the cell, thereby forming a second hole injection layer having a thickness of 5 nm on the first hole injection layer. Compound HT-1 was then introduced into a cell of the vacuum vapor deposition apparatus and was evaporated by applying an electric current to the cell, thereby forming a first hole transport layer having a thickness of 10 nm on the second hole injection layer. Compound HT-2 was then introduced into another cell of the vacuum vapor deposition apparatus and was evaporated by applying an electric current to the cell, thereby forming a second hole transport layer having a thickness of 60 nm on the first hole transport layer. After forming the hole injection layers and the hole transport layers, a light-emitting layer was formed thereon as follows: The first host compound and the second host compound shown in Table 1 below were introduced into two cells of the vacuum vapor depositing apparatus as a host, and compound D-39 was introduced into another cell as a dopant. The two host materials were evaporated at a rate of 1:1 and the dopant material was simultaneously evaporated at a different rate, and the dopant was deposited in a doping amount of 3 wt % based on the total amount of the host and the dopant to form a light-emitting layer having a thickness of 40 nm on the second hole transport layer. Compound ET-1 and compound EI-1 were introduced into two cells and evaporated at a rate of 1:1 to form an electron transport layer having a thickness of 35 nm on the light-emitting layer. After depositing compound EI-1 as an electron injection layer having a thickness of 2 nm on the electron transport layer, an Al cathode having a thickness of 80 nm was deposited on the electron injection layer by another vacuum vapor deposition apparatus. Thus, an OLED was produced.

### Device Examples 5 to 7: Producing an OLED Deposited with the Plurality of Host Materials According to the Present Disclosure as a Host

OLEDs were produced in the same manner as in Device Example 1, except that compound HT-3 was used to form a second hole transport layer having a thickness of 45 nm, and compound EB-1 was used to form an electron blocking layer having a thickness of 15 nm on the second hole transport layer.

### Comparative Examples 1 to 4: Producing an OLED Comprising the Comparative Compound as a Host

OLEDs were produced in the same manner as in Device Example 1, except that the compounds shown in Table 1 below were used as a host of the light-emitting layer.

The time taken for luminance to decrease from 100% to 95% at a luminance of 5,000 nit and at a constant current (lifespan; T95) of the OLEDs produced in Device Examples 1 to 15, and Comparative Examples 1 to 4 are provided in Table 1 below.

TABLE 1

| | | Second | Lifespan |
|---|---|---|---|
| | First Host | Host | T95(hr) |
| Comparative Example 1 | — | C-146 | 11 |
| Comparative Example 2 | — | C-491 | 19 |
| Comparative Example 3 | A-1 | C-146 | 76 |
| Comparative Example 4 | A-2 | C-146 | 14 |
| Device Example 1 | H-1-142 | C-146 | 344 |
| Device Example 2 | H-1-142 | C-489 | 581 |
| Device Example 3 | H-1-142 | C-491 | 517 |
| Device Example 4 | H-1-52 | C-146 | 230 |
| Device Example 5 | H-1-142 | C-491 | 323 |
| Device Example 6 | H-1-149 | C-491 | 240 |
| Device Example 7 | H-1-47 | C-491 | 246 |
| Device Example 8 | H-1-46 | C-489 | 261 |
| Device Example 9 | H-1-143 | C-489 | 206 |
| Device Example 10 | H-1-142 | C-13 | 648 |
| Device Example 11 | H-1-142 | C-167 | 215 |
| Device Example 12 | H-1-52 | C-174 | 169 |
| Device Example 13 | H-1-52 | C-518 | 207 |
| Device Example 14 | H-1-52 | C-527 | 230 |
| Device Example 15 | H-1-52 | C-520 | 405 |

From Table 1 above, it can be seen that the OLEDs comprising a specific combination of compounds according to the present disclosure as host materials have remarkably improved lifespan property, compared to the OLEDs comprising as ingle host material (Comparative Examples 1 and 2) or comprising the host compound according to the present disclosure in combination with the conventional host compound as a host material (Comparative Examples 3 and 4).

### Device Examples 16 to 21: Producing a Red OILED Deposited with the Plurality of Host Materials According to the Present Disclosure as a Host

OLEDs according to the present disclosure were produced. A transparent electrode indium tin oxide (ITO) thin film (10 Ω/sq) on a glass substrate for an OLED (GEOMATEC CO., LTD., Japan) was subjected to an ultrasonic washing with acetone and isoprolyl alcohol, sequentially, and then was stored in isopropyl alcohol. The ITO substrate was then mounted on a substrate holder of a vacuum vapor deposition apparatus. Compound HI-3 was introduced into a cell of the vacuum vapor deposition apparatus, and compound HT-1 was then introduced into another cell, and then the two materials were evaporated at a different rate to deposit compound HI-3 in a doping amount of 3 wt % based on the total amount of compound HI-3 and compound HT-1, thereby forming a hole injection layer having a thickness of 10 nm on the ITO substrate. Next, compound HT-1 was then deposited on the hole injection layer to form a first hole transport layer having a thickness of 80 nm. Compound HT-2 was then introduced into another cell of the vacuum vapor deposition apparatus and was evaporated by applying an electric current to the cell, thereby forming a second hole transport layer having a thickness of 60 nm on the first hole transport layer. After forming the hole injection layer and the hole transport layers, a light-emitting layer was formed thereon as follows: The first host compound and the second host compound shown in Table 2 below were introduced into two cells of the vacuum vapor depositing apparatus as a host, and compound D-39 was introduced into another cell as a dopant. The two host materials were evaporated at a rate of 1:1 and the dopant material was simultaneously evaporated at a different rate, and the dopant was deposited in a doping amount of 3 wt % based on the total amount of the host and the dopant to form a light-emitting layer having a thickness of 40 nm on the second hole transport layer. Compound ET-1 and compound EI-1 were introduced into two cells and evaporated at a weight ratio of 50:50 to form an electron transport layer having a thickness of 35 nm on the light-emitting layer. After depositing compound EI-1 as an electron injection layer having a thickness of 2 nm on the electron transport layer, an Al cathode having a thickness of 80 nm was deposited on the electron injection layer by another vacuum vapor deposition apparatus. Thus, an OLED was produced. All the materials used for producing the OLED were purified by vacuum sublimation at 10⁻⁶ torr.

### Comparative Examples 5 and 6: Producing an OLED Comprising the Comparative Compound as a Host

OLEDs were produced in the same manner as in Device Example 16, except that the compounds shown in Table 2 below were used as a host of the light-emitting layer.

The driving voltage, the luminous efficiency, and the emission color at a luminance of 1,000 nit, and the time taken for luminance to decrease from 100% to 95% at a luminance of 5,000 nit (lifespan; T95) of the OLEDs produced in Device Examples 16 to 21, and Comparative Examples 5 and 6 are provided in Table 2 below.

TABLE 2

| | | | Driv- | Lu- | | |
|---|---|---|---|---|---|---|
| | | | ing | minous | | Life- |
| | | | Vol- | Ef- | E- | span |
| | First | Second | tage | ficiency | mission | T95 |
| | Host | Host | (V) | (cd/A) | Color | (hr) |
| Device | H-1-142 | C-254 | 3.2 | 34.0 | Red | 163 |
| Example 16 | | | | | | |
| Device | H-1-125 | C-254 | 3.2 | 35.2 | Red | 198 |
| Example 17 | | | | | | |
| Device | H-1-142 | C-263 | 3.1 | 34.6 | Red | 199 |
| Example 18 | | | | | | |
| Device | H-1-125 | C-263 | 3.1 | 34.7 | Red | 185 |
| Example 19 | | | | | | |
| Device | H-1-142 | C-599 | 3.1 | 33.9 | Red | 272 |
| Example 20 | | | | | | |
| Device | H-1-125 | C-599 | 3.1 | 33.6 | Red | 254 |
| Example 21 | | | | | | |
| Comparative | | C-263 | 3.5 | 27.9 | Red | 38.2 |
| Example 5 | | | | | | |
| Comparative | | C-599 | 3.4 | 23.9 | Red | 19.5 |
| Example 6 | | | | | | |

From Table 2 above, it can be seen that the OLEDs comprising a specific combination of compounds according to the present disclosure as host materials show improved driving voltage, luminous efficiency, and/or lifespan properties compared to the comparative devices.

The compounds used in the Device Examples and the Comparative Examples are shown in Table 3 below.

TABLE 3

| | |
|---|---|
| Hole Injection Layer/ Hole Transport Layer | 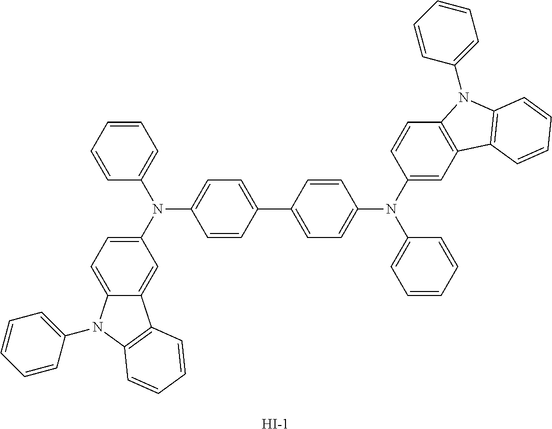 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; [I-]] |
| | 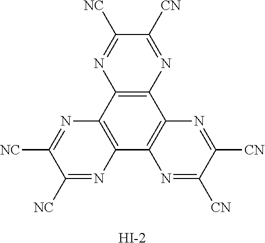 [SMILES: N#Cc1nc2c3nc(C#N)c(C#N)nc3c3nc(C#N)c(C#N)nc3c2nc1C#N ; "HI-2"] |
| | 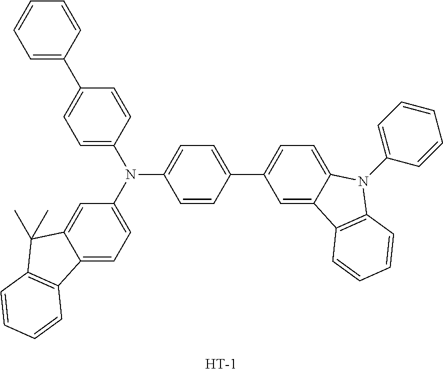 |
| | 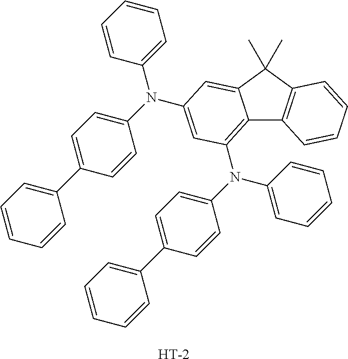 |
| | 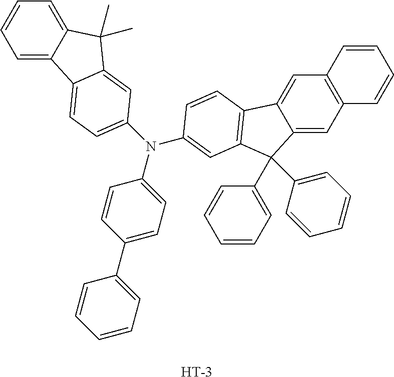 |
| | 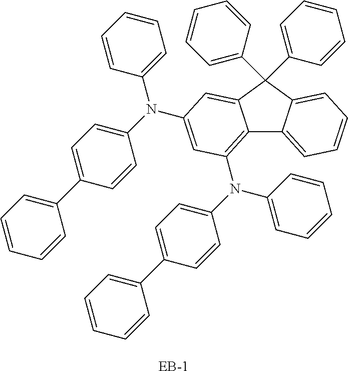 |
| | 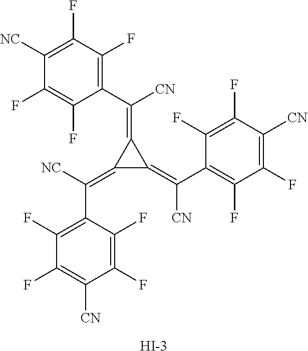 |
| Light-Emitting Layer | 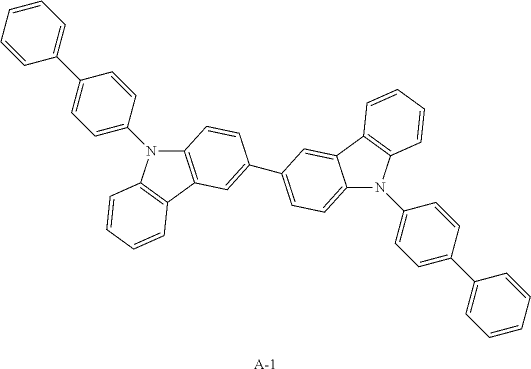 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; "A-1"] |
| | 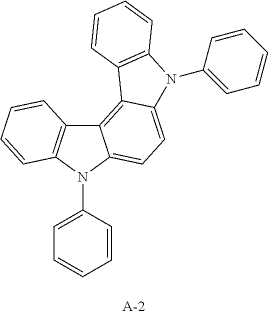 [SMILES: c1ccc(-n2c3ccccc3c3c4c5ccccc5n(-c5ccccc5)c4ccc32)cc1 ; "A-2"] |
| | 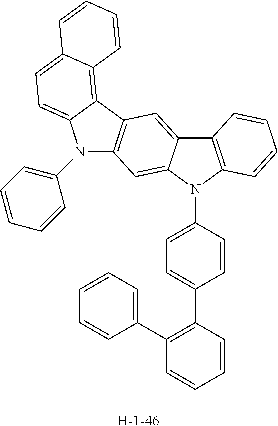 [SMILES: c1ccc(-c2ccccc2-c2ccc(-n3c4ccccc4c4cc5c6c7ccccc7ccc6n(-c6ccccc6)c5cc43)cc2)cc1 ; "H-1-46"] |
| | 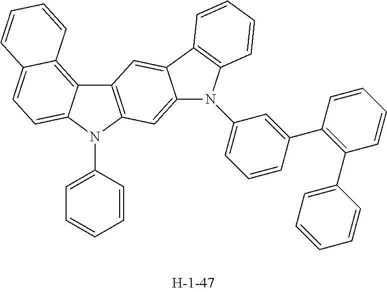 [SMILES: c1ccc(-c2ccccc2-c2cccc(-n3c4ccccc4c4cc5c6c7ccccc7ccc6n(-c6ccccc6)c5cc43)c2)cc1] |
| | 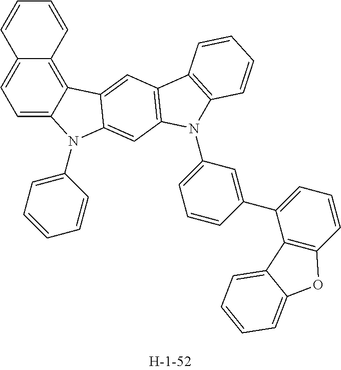 [SMILES: c1ccc(-n2c3cc4c(cc3c3c5ccccc5ccc32)c2ccccc2n4-c2cccc(-c3cccc4oc5ccccc5c34)c2)cc1] |
| | 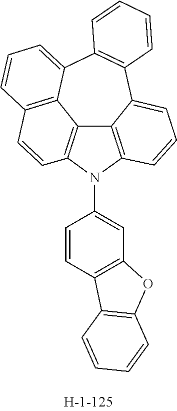 [SMILES: c1ccc2c(c1)-c1cccc3ccc4c(c13)c1c-2cccc1n4-c1ccc2c(c1)oc1ccccc12] |
| | 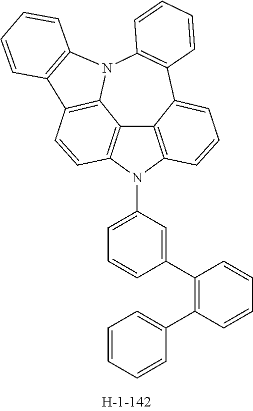 [SMILES: c1ccc(-c2ccccc2-c2cccc(-n3c4cccc5c6ccccc6n6c7ccccc7c7ccc3c(c54)c76)c2)cc1] |
| | 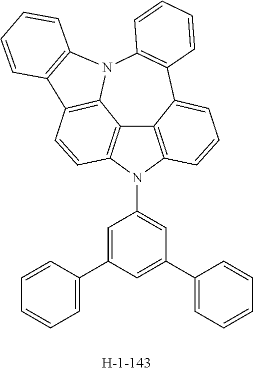 [SMILES: c1ccc(-c2cc(-c3ccccc3)cc(-n3c4cccc5c6ccccc6n6c7ccccc7c7ccc3c(c54)c76)c2)cc1] |
| | 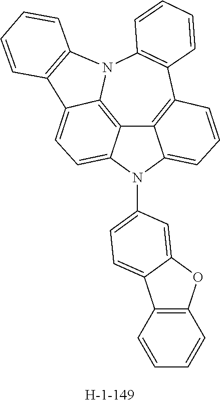 [SMILES: c1ccc2c(c1)oc1cc(-n3c4cccc5c6ccccc6n6c7ccccc7c7ccc3c(c54)c76)ccc12] |
| | 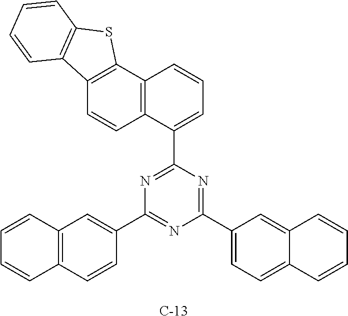 |
| | 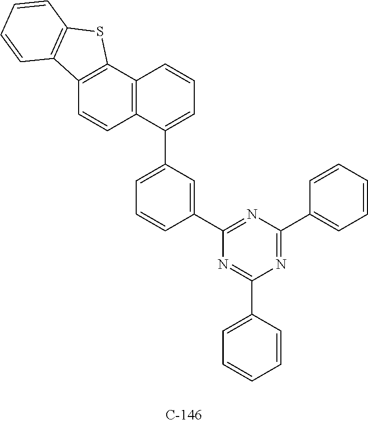 [SMILES: c1ccc(-c2nc(-c3ccccc3)nc(-c3cccc(-c4cccc5c4ccc4c6ccccc6sc54)c3)n2)cc1] |
| | 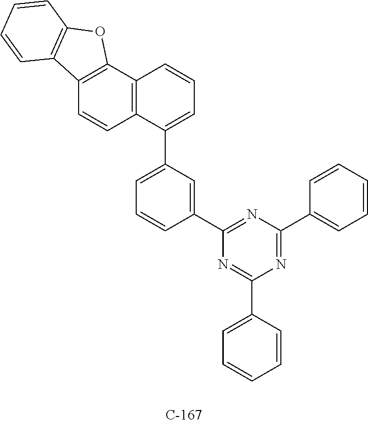 [SMILES: c1ccc(-c2nc(-c3ccccc3)nc(-c3cccc(-c4cccc5c4ccc4c6ccccc6oc54)c3)n2)cc1] |
| | 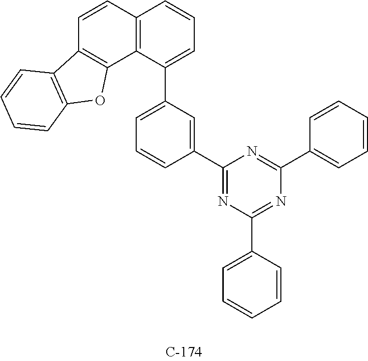 [SMILES: c1ccc(-c2nc(-c3ccccc3)nc(-c3cccc(-c4cccc5ccc6c7ccccc7oc6c45)c3)n2)cc1] |
| | 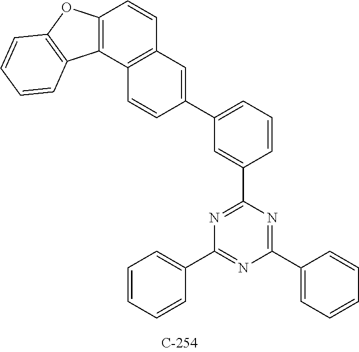 [SMILES: c1ccc(-c2nc(-c3ccccc3)nc(-c3cccc(-c4ccc5c(ccc6oc7ccccc7c65)c4)c3)n2)cc1] |
| | 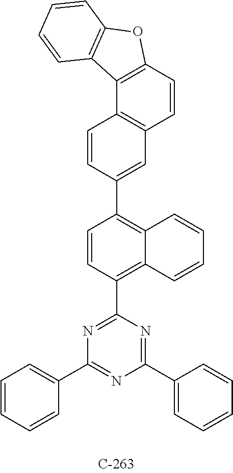 [SMILES: c1ccc(-c2nc(-c3ccccc3)nc(-c3ccc(-c4ccc5c(ccc6oc7ccccc7c65)c4)c4ccccc34)n2)cc1] |
| | 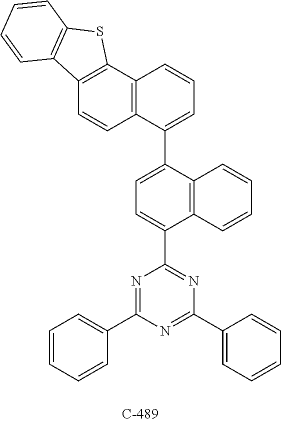 [SMILES: c1ccc(-c2nc(-c3ccccc3)nc(-c3ccc(-c4cccc5c4ccc4c6ccccc6sc54)c4ccccc34)n2)cc1] |
| | 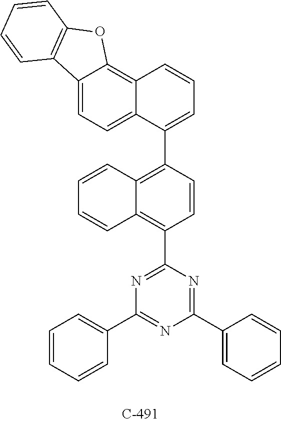 [SMILES: c1ccc(-c2nc(-c3ccccc3)nc(-c3ccc(-c4cccc5c4ccc4c6ccccc6oc54)c4ccccc34)n2)cc1] |
| | 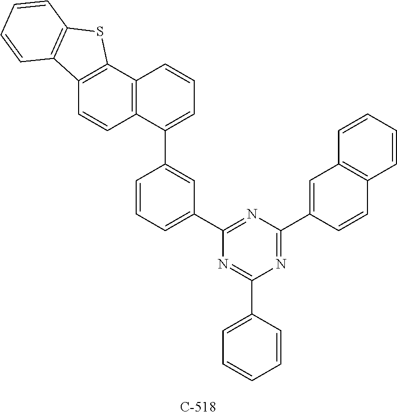 [SMILES: c1ccc(-c2nc(-c3cccc(-c4cccc5c4ccc4c6ccccc6sc54)c3)nc(-c3ccc4ccccc4c3)n2)cc1] |
| | 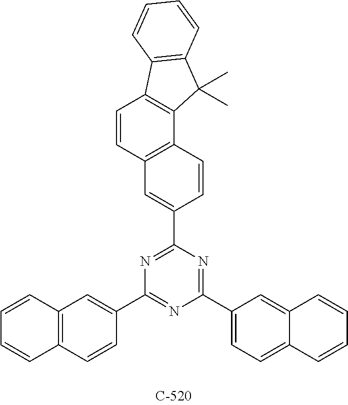 [SMILES: CC1(C)c2ccccc2-c2ccc3cc(-c4nc(-c5ccc6ccccc6c5)nc(-c5ccc6ccccc6c5)n4)ccc3c21] |
| | 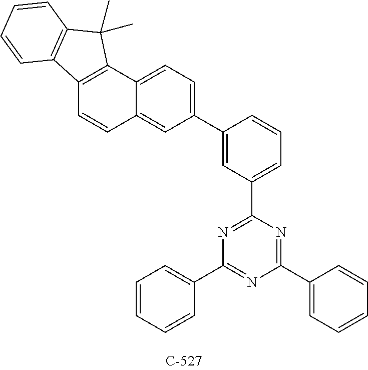 [SMILES: CC1(C)c2ccccc2-c2ccc3cc(-c4cccc(-c5nc(-c6ccccc6)nc(-c6ccccc6)n5)c4)ccc3c21] |
| | 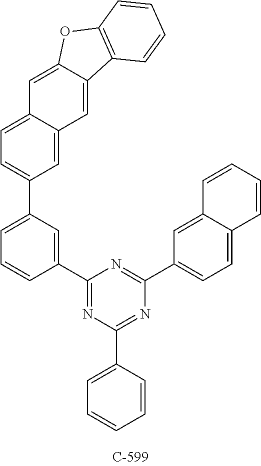 [SMILES: c1ccc(-c2nc(-c3cccc(-c4ccc5cc6oc7ccccc7c6cc5c4)c3)nc(-c3ccc4ccccc4c3)n2)cc1] |
| | 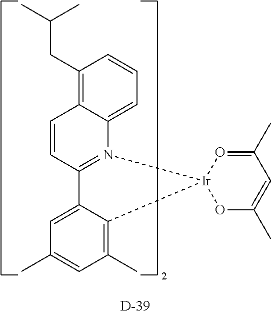 |
| Electron Transport Layer/ Electron Injection Layer | 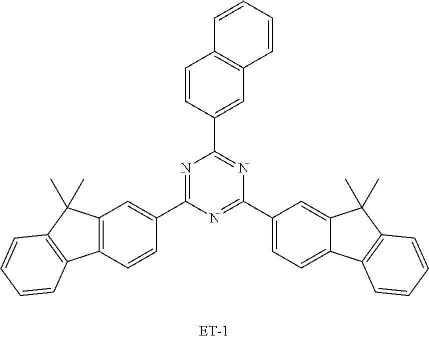 [SMILES: CC1(C)c2ccccc2-c2ccc(-c3nc(-c4ccc5c(c4)C(C)(C)c4ccccc4-5)nc(-c4ccc5ccccc5c4)n3)cc21] |
| | 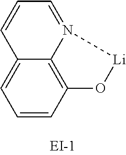 |

1. A plurality of host materials comprising a first host material comprising a compound represented by the following formula 1, and a second host material comprising a compound represented by the following formula 2: 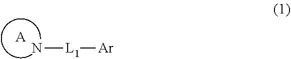
wherein
L₁ represents a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene;
Ar represents hydrogen, deuterium, a halogen, a cyano, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, a substituted or unsubstituted (3- to 30-membered)heteroaryl, —NR₁R₂, or —SiR₃R₄R₅;
R₁ to R₅, each independently, represent a substituted or unsubstituted (C1-C30)alkyl, a substituted or unsubstituted (C6-C30)aryl, or a substituted or unsubstituted (3- to 30-membered)heteroaryl; 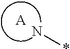
is represented by any one of the following formulas 1-1 to 1-7: 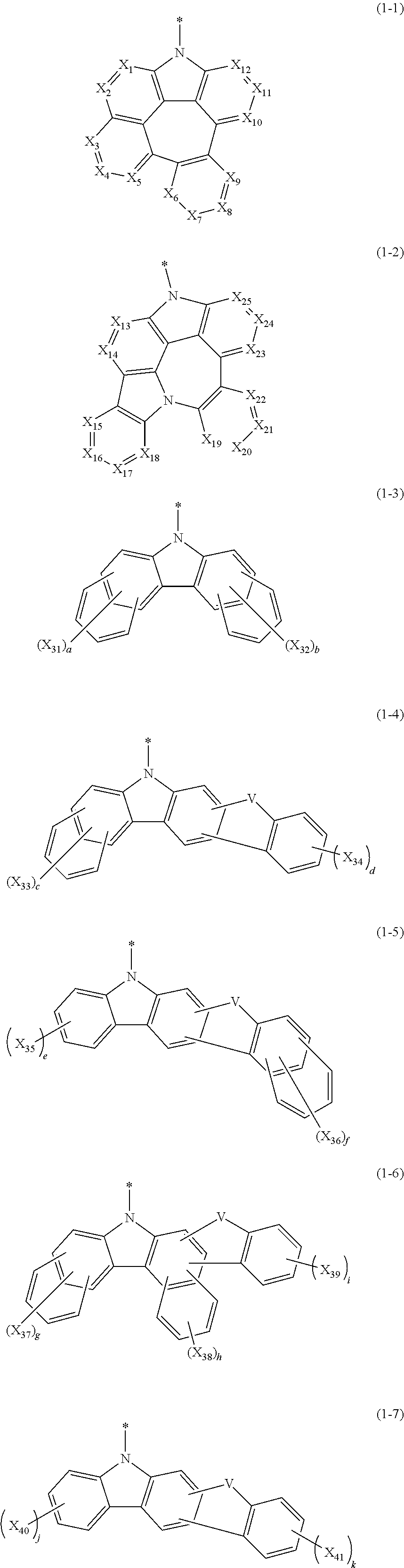
X₁ to X₂₅, each independently, represent N or CR₆;
R₆, each independently, represents hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or adjacent R₆'s may be linked to each other to form a ring(s), and if two or more R₆'s are present, each of R₆ may be the same or different;
V represents CX₂₆X₂₇, NX₂₈, O, or S;
X₂₆ to X₂₈ and X₃₁ to X₄₁, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or each of X₃₁ to X₄₁ may be linked to an adjacent substituent of X₃₁ to X₄₁ to form a ring(s);
a, b, c, f, and g, each independently, represent an integer of 1 to 6, and d, e, h, i, j, and k, each independently, represent an integer of 1 to 4, where if a to k are an integer equal to 2 or more, each of X₃₁ to each of X₄₁ may be the same or different; and
* represents a bonding site to L₁; 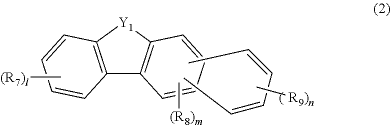
wherein
Y₁ represents O, S, CR₁₁R₁₂, or NR₁₃;
R₁₁ to R₁₃, each independently, represent hydrogen, deuterium, a halogen, a cyano, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, or a substituted or unsubstituted (3- to 30-membered)heteroaryl; or R₁₁ and R₁₂ may be linked to each other to form a ring;
R₇ to R₉, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted (C1-C30)alkyl(C6-C30)arylamino, or a substituted or unsubstituted mono- or di-(C6-C30)arylamino; or may be linked to an adjacent substituent to form a ring(s);
with the proviso that at least one of R₁₃, R₈, and R₉ represents -L₂-(Ar₁)ₚ;
L₂ represents a single bond, a substituted or unsubstituted (C6-C30)arylene, or a substituted or unsubstituted (3- to 30-membered)heteroarylene;
Ar₁, each independently, represents a substituted or unsubstituted (3- to 30-membered)heteroaryl containing at least one nitrogen; and
l and n, each independently, represent an integer of 1 to 4, and m and p, each independently, represent an integer of 1 or 2, where if l to n and p are an integer equal to 2 or more, each of R₇, each of R₈, each of R₉, and each of Ar₁ may be the same or different.

2. The plurality of host materials according to claim 1, wherein the substituents of the substituted alkyl(ene), the substituted aryl(ene), the substituted heteroaryl(ene), the substituted cycloalkyl(ene), the substituted cycloalkenyl, the substituted heterocycloalkyl, the substituted alkoxy, the substituted trialkylsilyl, the substituted dialkylarylsilyl, the substituted alkyldiarylsilyl, the substituted triarylsilyl, the substituted mono- or di-alkylamino, the substituted mono- or di-arylamino, and the substituted alkylarylamino in R₁ to R₉, R₁₁ to R₁₃, L₁, L₂, Ar, Ar₁, X₂₆ to X₂₈, and X₃₁ to X₄₁, each independently, are at least one selected from the group consisting of deuterium; a halogen; a cyano; a carboxyl; a nitro; a hydroxyl; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 30-membered)heteroaryl unsubstituted or substituted with a (C6-C30)aryl(s); a (C6-C30)aryl unsubstituted or substituted with at least one of a (C1-C30)alkyl(s) and a (3- to 30-membered)heteroaryl(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; an amino; a mono- or di-(C1-C30)alkylamino; a mono- or di-(C6-C30)arylamino unsubstituted or substituted with a (C1-C30)alkyl(s); a (C1-C30)alkyl(C6-C30)arylamino; a (C1-C30)alkylcarbonyl; a (C1-C30)alkoxycarbonyl; a (C6-C30)arylcarbonyl; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl.

3. The plurality of host materials according to claim 1, wherein formula 2 is represented by at least one of the following formulas 2-1 to 2-9: 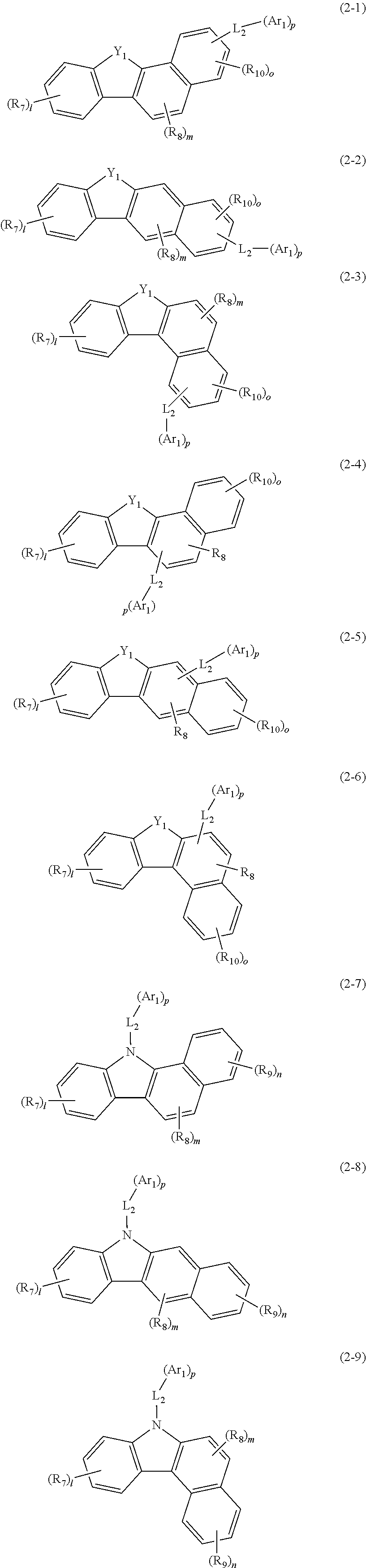
wherein
Y₁, L₂, Ar₁, R₇ to R₉, l to n, and p are as defined in claim 1;
R₁₀, each independently, has the same definition as R₉; and
o represents an integer of 1 to 3, where if o is an integer equal to 2 or more, each of R₁₀ may be the same or different.

4. The plurality of host materials according to claim 1, wherein Ar₁, each independently, represents a substituted or unsubstituted pyridyl, a substituted or unsubstituted pyrimidinyl, a substituted or unsubstituted triazinyl, a substituted or unsubstituted pyrazinyl, a substituted or unsubstituted pyridazinyl, a substituted or unsubstituted quinazolinyl, a substituted or unsubstituted benzoquinazolinyl, a substituted or unsubstituted quinoxalinyl, a substituted or unsubstituted benzoquinoxalinyl, a substituted or unsubstituted quinolyl, a substituted or unsubstituted benzoquinolyl, a substituted or unsubstituted isoquinolyl, a substituted or unsubstituted benzoisoquinolyl, a substituted or unsubstituted triazolyl, a substituted or unsubstituted pyrazolyl, a substituted or unsubstituted naphthyridinyl, or a substituted or unsubstituted benzothienopyrimidinyl.

5. The plurality of host materials according to claim 1, wherein the compound represented by formula 1 is at least one selected from the following compounds:

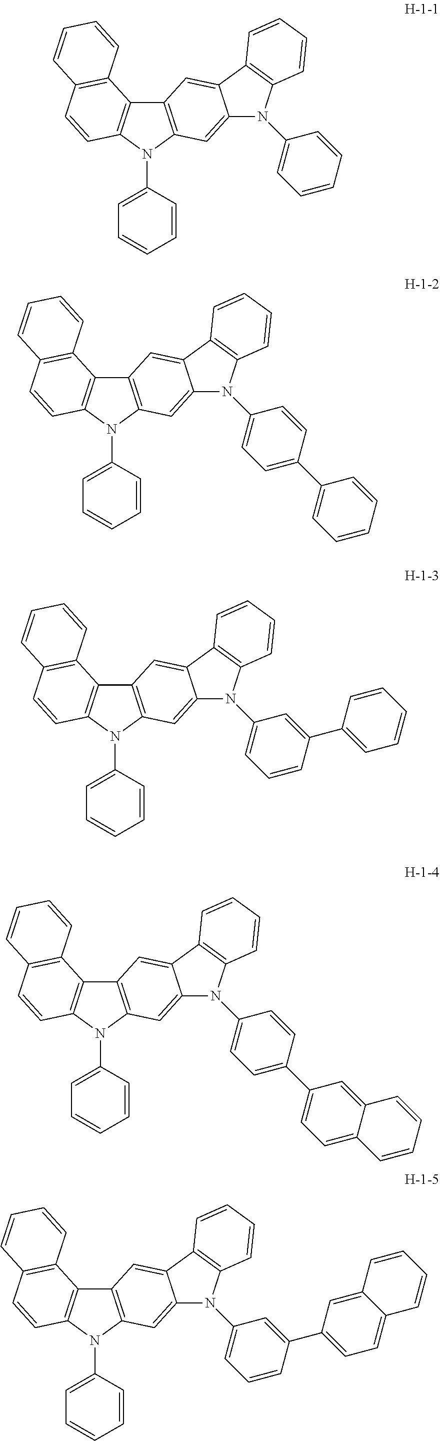 [SMILES: c1ccc(-n2c3ccccc3c3cc4c5c6ccccc6ccc5n(-c5ccccc5)c4cc32)cc1 ; c1ccc(-c2ccc(-n3c4ccccc4c4cc5c6c7ccccc7ccc6n(-c6ccccc6)c5cc43)cc2)cc1 ; c1ccc(-c2cccc(-n3c4ccccc4c4cc5c6c7ccccc7ccc6n(-c6ccccc6)c5cc43)c2)cc1 ; c1ccc(-n2c3cc4c(cc3c3c5ccccc5ccc32)c2ccccc2n4-c2ccc(-c3ccc4ccccc4c3)cc2)cc1 ; c1ccc(-n2c3cc4c(cc3c3c5ccccc5ccc32)c2ccccc2n4-c2cccc(-c3ccc4ccccc4c3)c2)cc1]

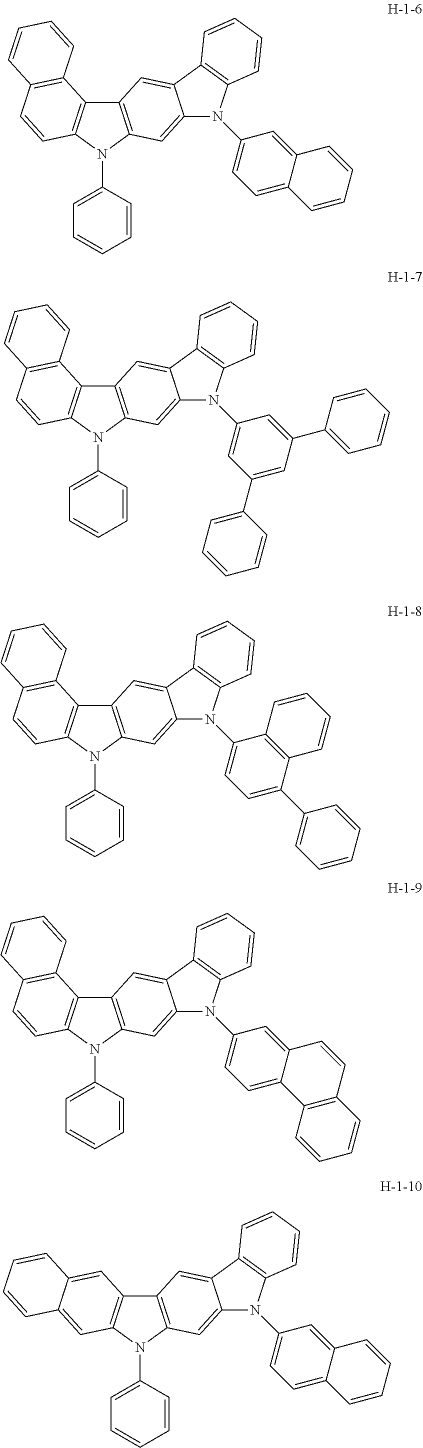

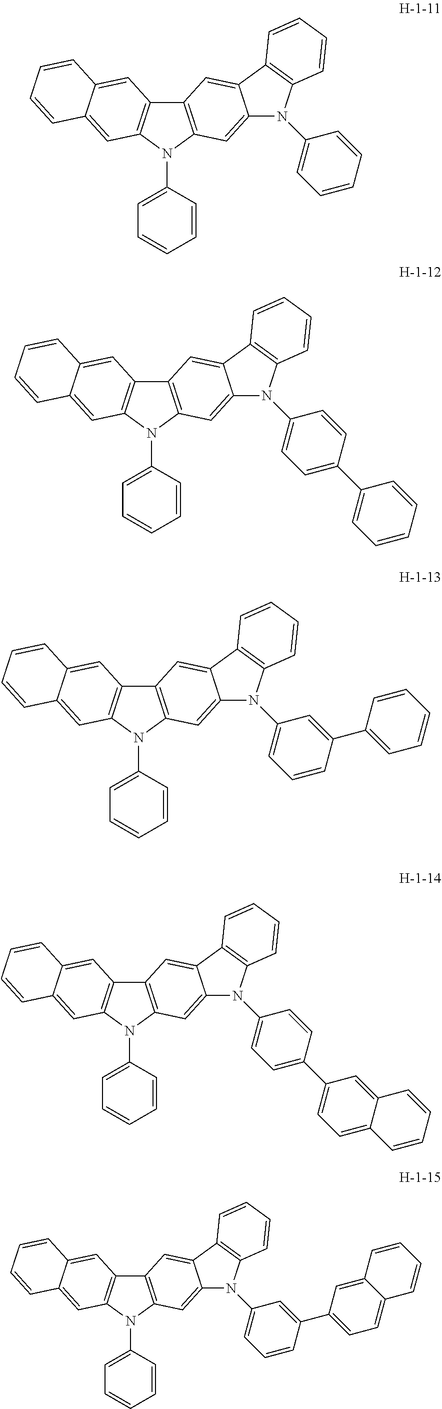

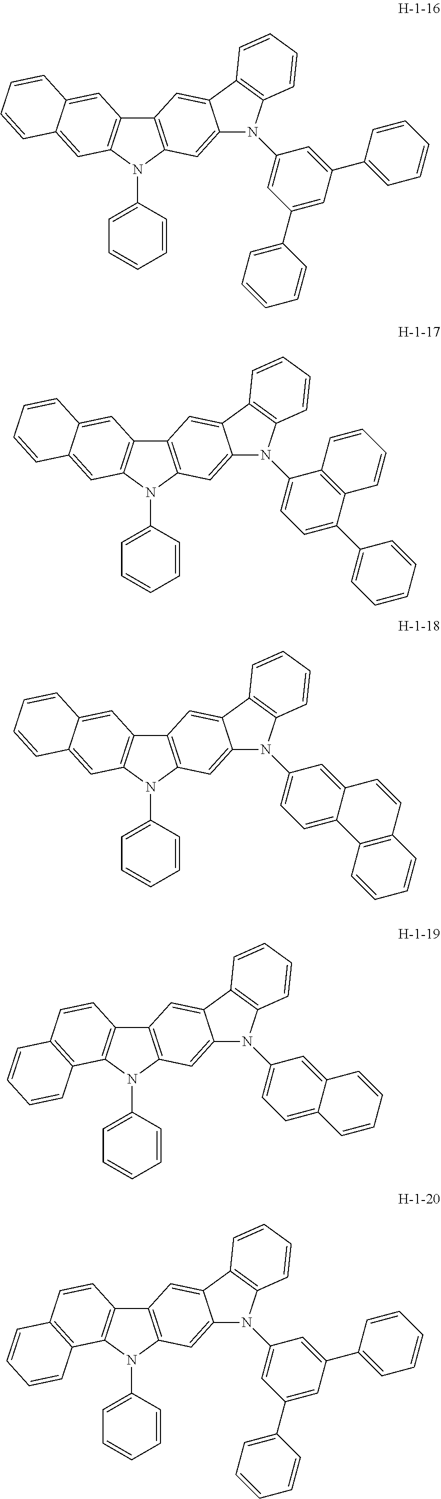 [SMILES: c1ccc(-c2cc(-c3ccccc3)cc(-n3c4ccccc4c4cc5c6cc7ccccc7cc6n(-c6ccccc6)c5cc43)c2)cc1 ; c1ccc(-c2ccc(-n3c4ccccc4c4cc5c6cc7ccccc7cc6n(-c6ccccc6)c5cc43)c3ccccc23)cc1 ; c1ccc(-n2c3cc4ccccc4cc3c3cc4c5ccccc5n(-c5ccc6c(ccc7ccccc76)c5)c4cc32)cc1 ; c1ccc(-n2c3cc4c(cc3c3ccc5ccccc5c32)c2ccccc2n4-c2ccc3ccccc3c2)cc1 ; c1ccc(-c2cc(-c3ccccc3)cc(-n3c4ccccc4c4cc5c6ccc7ccccc7c6n(-c6ccccc6)c5cc43)c2)cc1]

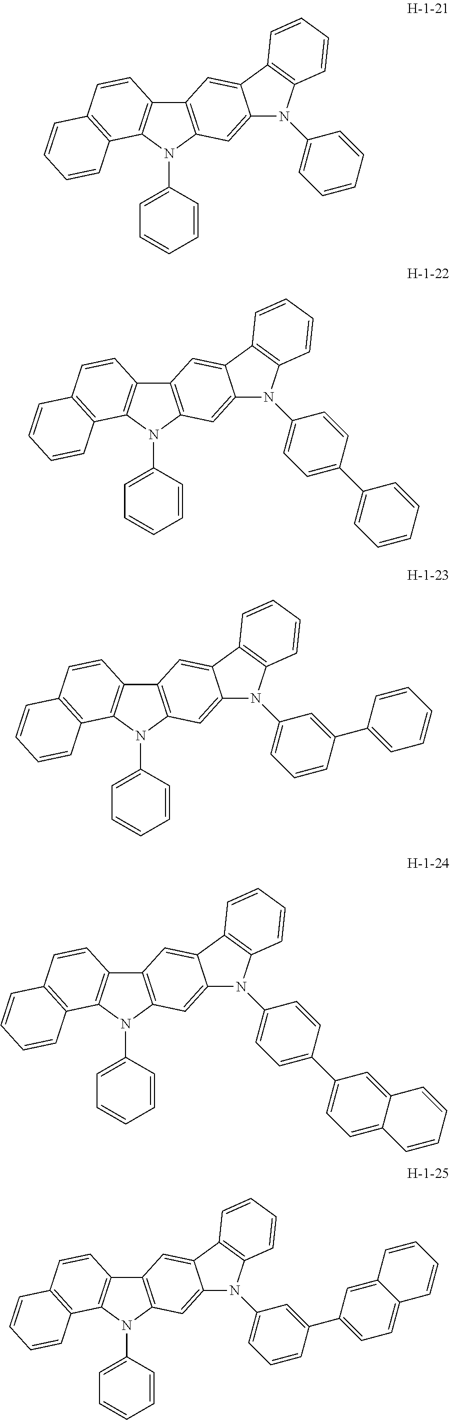 [SMILES: c1ccc(-n2c3ccccc3c3cc4c5ccc6ccccc6c5n(-c5ccccc5)c4cc32)cc1 ; c1ccc(-c2ccc(-n3c4ccccc4c4cc5c6ccc7ccccc7c6n(-c6ccccc6)c5cc43)cc2)cc1 ; c1ccc(-c2cccc(-n3c4ccccc4c4cc5c6ccc7ccccc7c6n(-c6ccccc6)c5cc43)c2)cc1 ; c1ccc(-n2c3cc4c(cc3c3ccc5ccccc5c32)c2ccccc2n4-c2ccc(-c3ccc4ccccc4c3)cc2)cc1 ; c1ccc(-n2c3cc4c(cc3c3ccc5ccccc5c32)c2ccccc2n4-c2cccc(-c3ccc4ccccc4c3)c2)cc1]

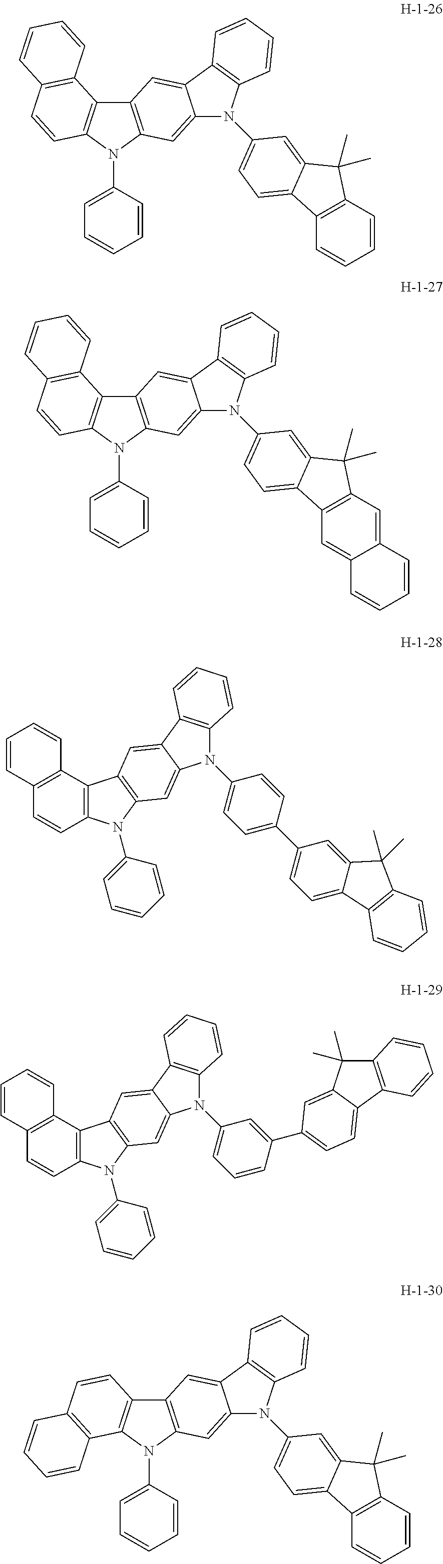 [SMILES: CC1(C)c2ccccc2-c2ccc(-n3c4ccccc4c4cc5c6c7ccccc7ccc6n(-c6ccccc6)c5cc43)cc21 ; CC1(C)c2cc(-n3c4ccccc4c4cc5c6c7ccccc7ccc6n(-c6ccccc6)c5cc43)ccc2-c2cc3ccccc3cc21 ; CC1(C)c2ccccc2-c2ccc(-c3ccc(-n4c5ccccc5c5cc6c7c8ccccc8ccc7n(-c7ccccc7)c6cc54)cc3)cc21 ; CC1(C)c2ccccc2-c2ccc(-c3cccc(-n4c5ccccc5c5cc6c7c8ccccc8ccc7n(-c7ccccc7)c6cc54)c3)cc21 ; CC1(C)c2ccccc2-c2ccc(-n3c4ccccc4c4cc5c6ccc7ccccc7c6n(-c6ccccc6)c5cc43)cc21]

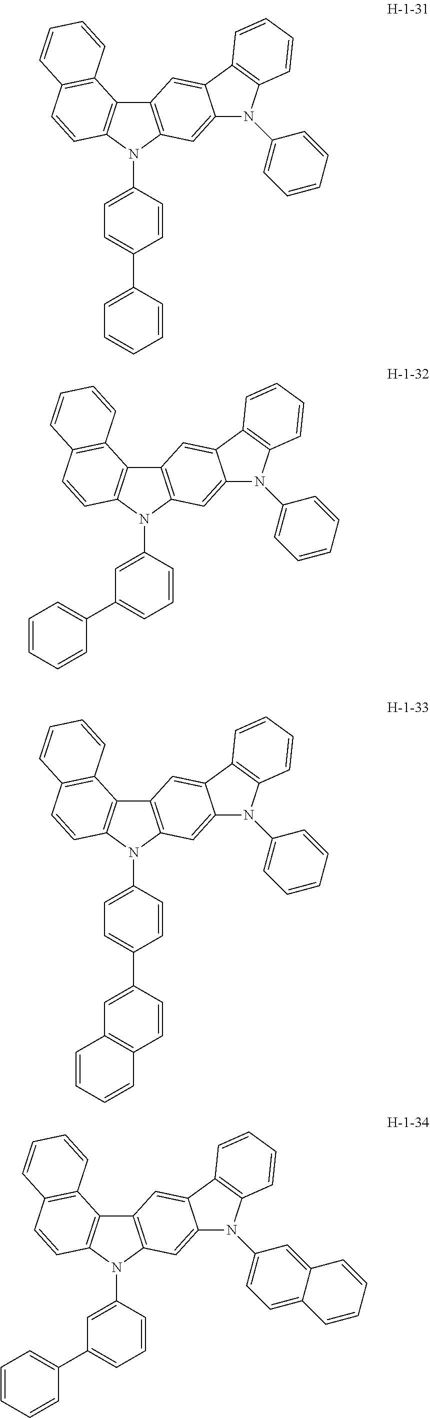 [SMILES: c1ccc(-c2ccc(-n3c4cc5c(cc4c4c6ccccc6ccc43)c3ccccc3n5-c3ccccc3)cc2)cc1 ; c1ccc(-c2cccc(-n3c4cc5c(cc4c4c6ccccc6ccc43)c3ccccc3n5-c3ccccc3)c2)cc1 ; c1ccc(-n2c3ccccc3c3cc4c5c6ccccc6ccc5n(-c5ccc(-c6ccc7ccccc7c6)cc5)c4cc32)cc1 ; c1ccc(-c2cccc(-n3c4cc5c(cc4c4c6ccccc6ccc43)c3ccccc3n5-c3ccc4ccccc4c3)c2)cc1]

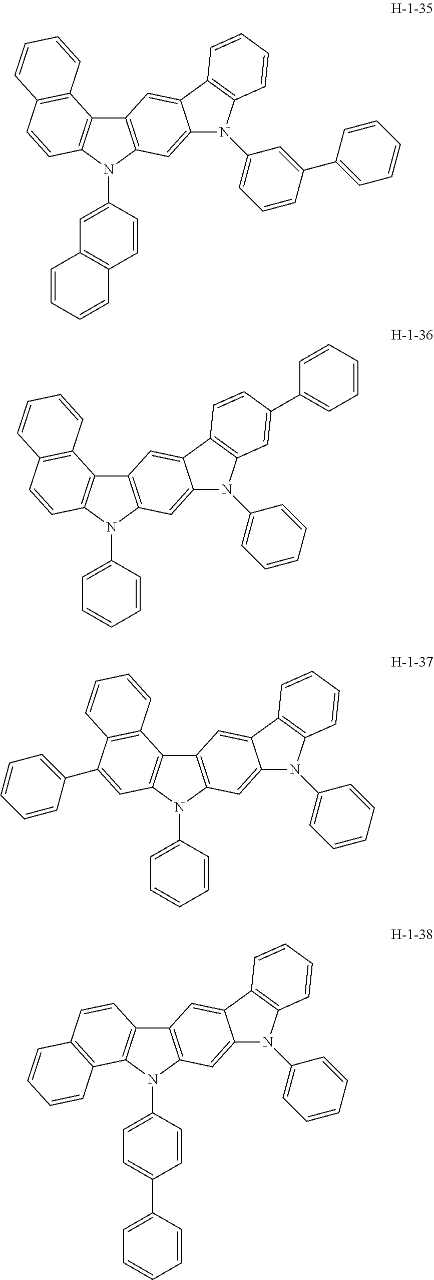 [SMILES: c1ccc(-c2cccc(-n3c4ccccc4c4cc5c6c7ccccc7ccc6n(-c6ccc7ccccc7c6)c5cc43)c2)cc1 ; c1ccc(-c2ccc3c4cc5c6c7ccccc7ccc6n(-c6ccccc6)c5cc4n(-c4ccccc4)c3c2)cc1 ; c1ccc(-c2cc3c(c4ccccc24)c2cc4c5ccccc5n(-c5ccccc5)c4cc2n3-c2ccccc2)cc1 ; c1ccc(-c2ccc(-n3c4cc5c(cc4c4ccc6ccccc6c43)c3ccccc3n5-c3ccccc3)cc2)cc1]

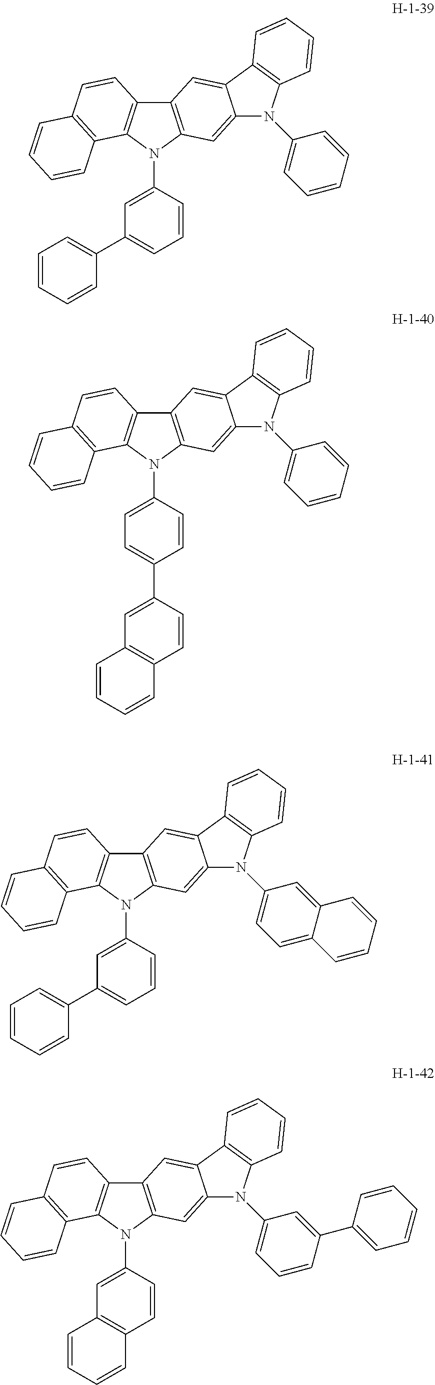 [SMILES: c1ccc(-c2cccc(-n3c4cc5c(cc4c4ccc6ccccc6c43)c3ccccc3n5-c3ccccc3)c2)cc1 ; c1ccc(-n2c3ccccc3c3cc4c5ccc6ccccc6c5n(-c5ccc(-c6ccc7ccccc7c6)cc5)c4cc32)cc1 ; c1ccc(-c2cccc(-n3c4cc5c(cc4c4ccc6ccccc6c43)c3ccccc3n5-c3ccc4ccccc4c3)c2)cc1 ; c1ccc(-c2cccc(-n3c4ccccc4c4cc5c6ccc7ccccc7c6n(-c6ccc7ccccc7c6)c5cc43)c2)cc1]

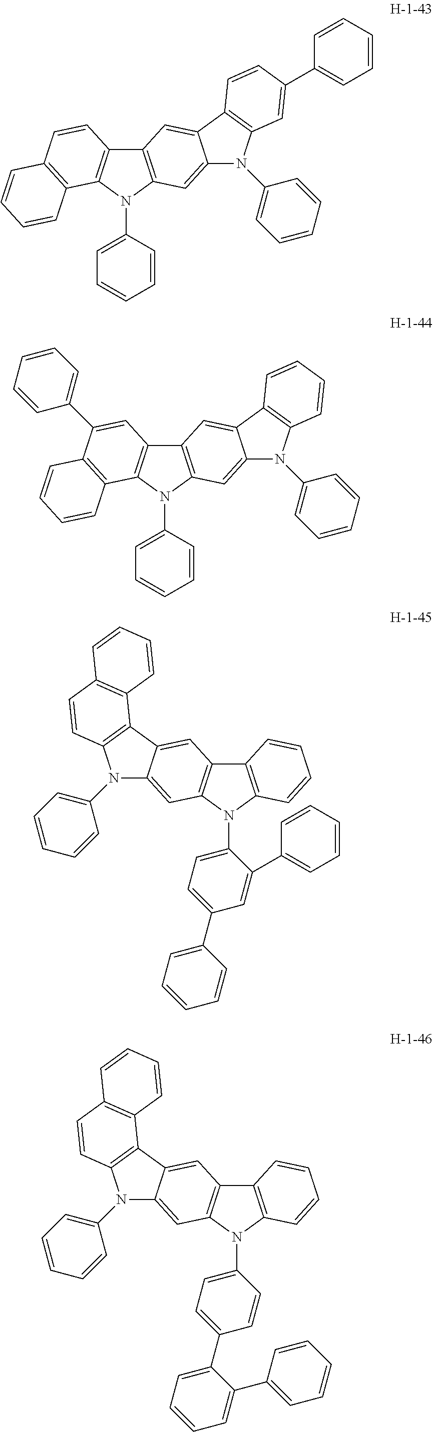

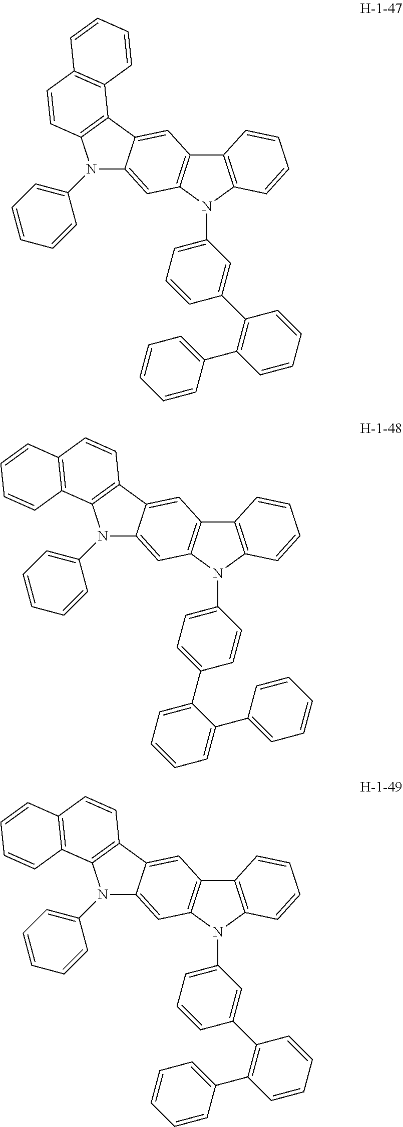 [SMILES: c1ccc(-c2ccccc2-c2cccc(-n3c4ccccc4c4cc5c6c7ccccc7ccc6n(-c6ccccc6)c5cc43)c2)cc1 ; c1ccc(-c2ccccc2-c2ccc(-n3c4ccccc4c4cc5c6ccc7ccccc7c6n(-c6ccccc6)c5cc43)cc2)cc1 ; c1ccc(-c2ccccc2-c2cccc(-n3c4ccccc4c4cc5c6ccc7ccccc7c6n(-c6ccccc6)c5cc43)c2)cc1]

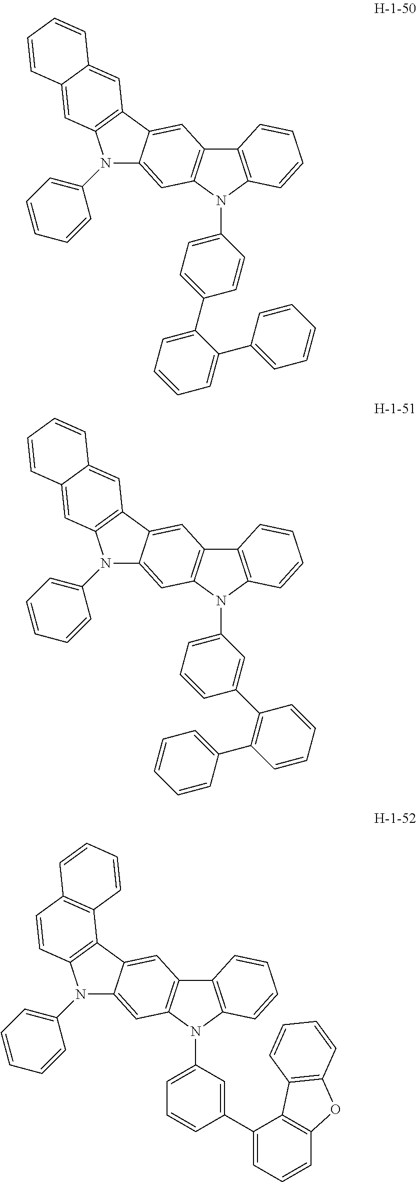 [SMILES: c1ccc(-c2ccccc2-c2ccc(-n3c4ccccc4c4cc5c6cc7ccccc7cc6n(-c6ccccc6)c5cc43)cc2)cc1 ; c1ccc(-c2ccccc2-c2cccc(-n3c4ccccc4c4cc5c6cc7ccccc7cc6n(-c6ccccc6)c5cc43)c2)cc1 ; c1ccc(-n2c3cc4c(cc3c3c5ccccc5ccc32)c2ccccc2n4-c2cccc(-c3cccc4oc5ccccc5c34)c2)cc1]

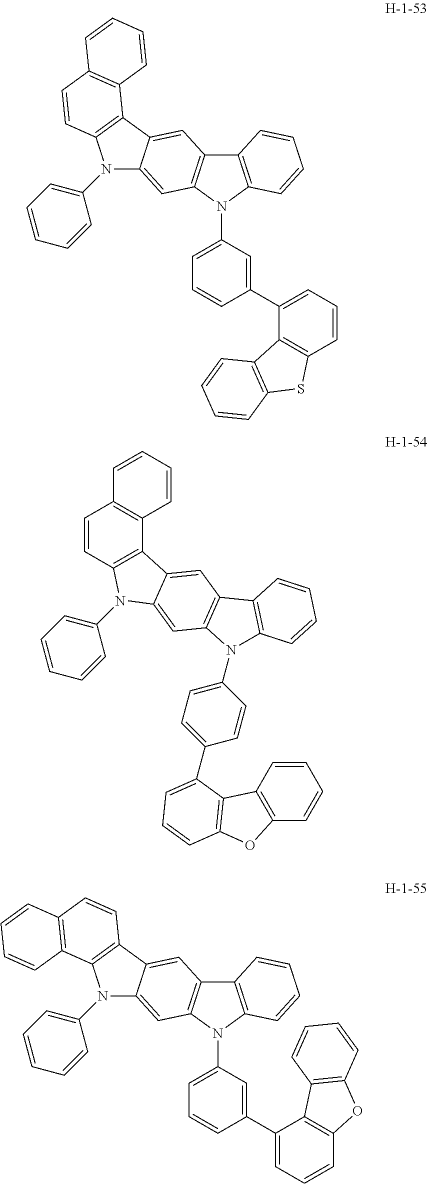 [SMILES: c1ccc(-n2c3cc4c(cc3c3c5ccccc5ccc32)c2ccccc2n4-c2cccc(-c3cccc4sc5ccccc5c34)c2)cc1 ; c1ccc(-n2c3cc4c(cc3c3c5ccccc5ccc32)c2ccccc2n4-c2ccc(-c3cccc4oc5ccccc5c34)cc2)cc1 ; c1ccc(-n2c3cc4c(cc3c3ccc5ccccc5c32)c2ccccc2n4-c2cccc(-c3cccc4oc5ccccc5c34)c2)cc1]

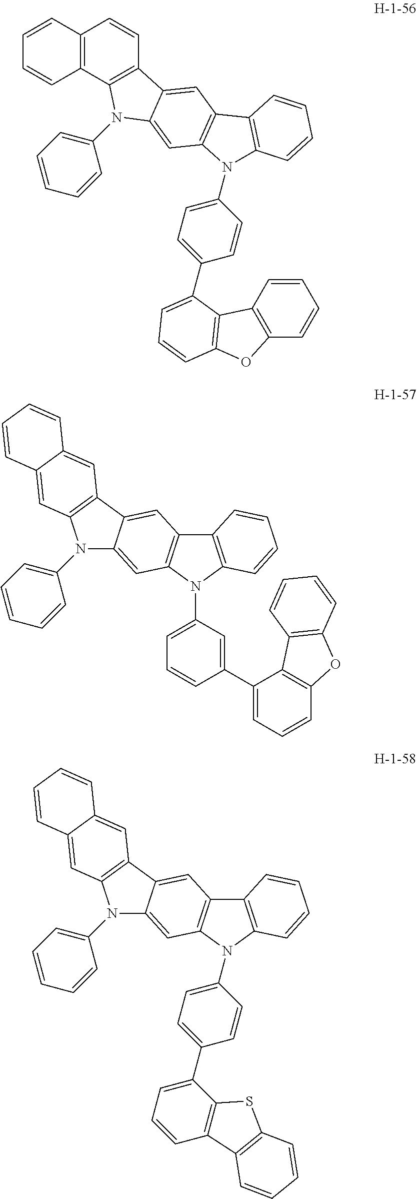 [SMILES: c1ccc(-n2c3cc4c(cc3c3ccc5ccccc5c32)c2ccccc2n4-c2ccc(-c3cccc4oc5ccccc5c34)cc2)cc1 ; c1ccc(-n2c3cc4ccccc4cc3c3cc4c5ccccc5n(-c5cccc(-c6cccc7oc8ccccc8c67)c5)c4cc32)cc1 ; c1ccc(-n2c3cc4ccccc4cc3c3cc4c5ccccc5n(-c5ccc(-c6cccc7c6sc6ccccc67)cc5)c4cc32)cc1]

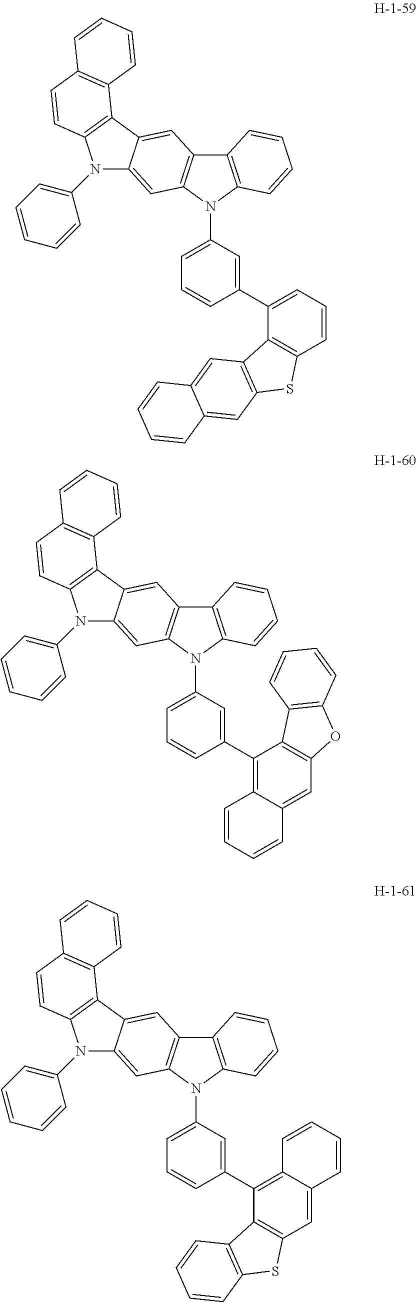 [SMILES: c1ccc(-n2c3cc4c(cc3c3c5ccccc5ccc32)c2ccccc2n4-c2cccc(-c3cccc4sc5cc6ccccc6cc5c34)c2)cc1 ; c1ccc(-n2c3cc4c(cc3c3c5ccccc5ccc32)c2ccccc2n4-c2cccc(-c3c4ccccc4cc4oc5ccccc5c34)c2)cc1 ; c1ccc(-n2c3cc4c(cc3c3c5ccccc5ccc32)c2ccccc2n4-c2cccc(-c3c4ccccc4cc4sc5ccccc5c34)c2)cc1]

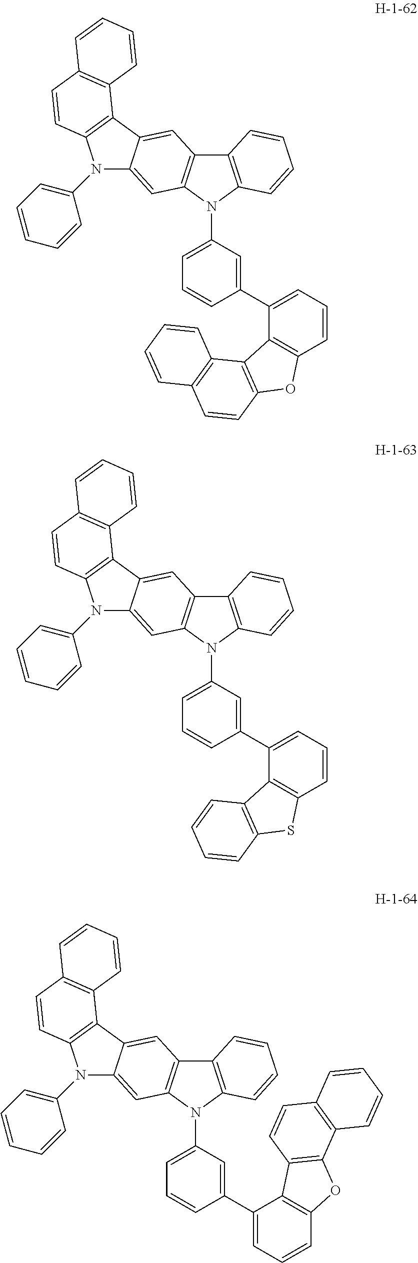 [SMILES: c1ccc(-n2c3cc4c(cc3c3c5ccccc5ccc32)c2ccccc2n4-c2cccc(-c3cccc4oc5ccc6ccccc6c5c34)c2)cc1 ; c1ccc(-n2c3cc4c(cc3c3c5ccccc5ccc32)c2ccccc2n4-c2cccc(-c3cccc4sc5ccccc5c34)c2)cc1 ; c1ccc(-n2c3cc4c(cc3c3c5ccccc5ccc32)c2ccccc2n4-c2cccc(-c3cccc4oc5c6ccccc6ccc5c34)c2)cc1]

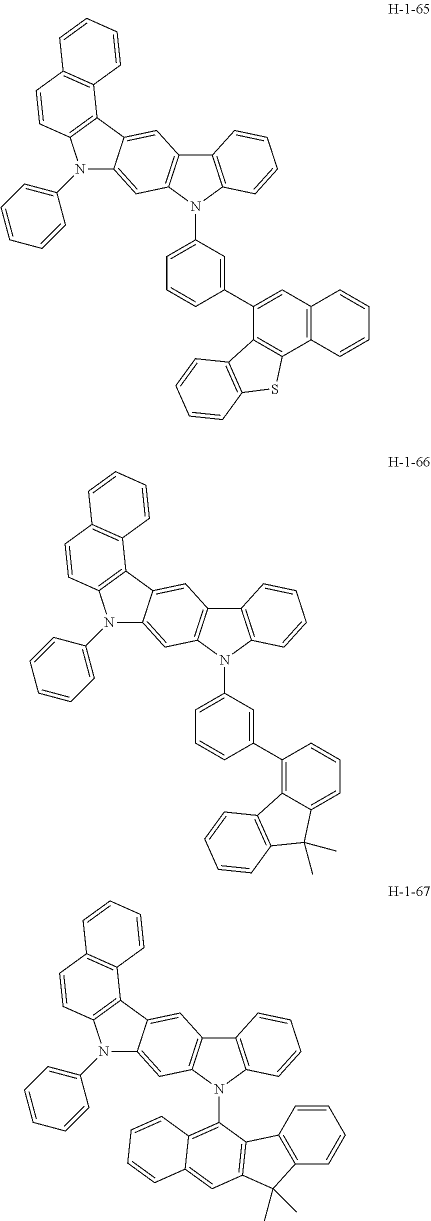 [SMILES: c1ccc(-n2c3cc4c(cc3c3c5ccccc5ccc32)c2ccccc2n4-c2cccc(-c3cc4ccccc4c4sc5ccccc5c34)c2)cc1 ; CC1(C)c2ccccc2-c2c(-c3cccc(-n4c5ccccc5c5cc6c7c8ccccc8ccc7n(-c7ccccc7)c6cc54)c3)cccc21 ; CC1(C)c2ccccc2-c2c1cc1ccccc1c2-n1c2ccccc2c2cc3c4c5ccccc5ccc4n(-c4ccccc4)c3cc21]

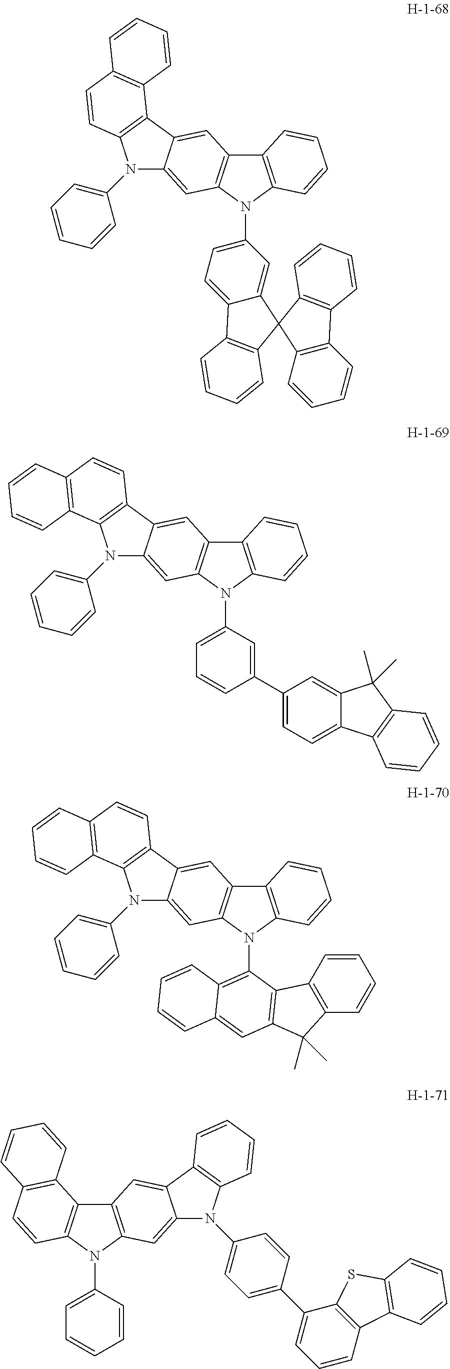 [SMILES: c1ccc(-n2c3cc4c(cc3c3c5ccccc5ccc32)c2ccccc2n4-c2ccc3c(c2)C2(c4ccccc4-c4ccccc42)c2ccccc2-3)cc1 ; CC1(C)c2ccccc2-c2ccc(-c3cccc(-n4c5ccccc5c5cc6c7ccc8ccccc8c7n(-c7ccccc7)c6cc54)c3)cc21 ; CC1(C)c2ccccc2-c2c1cc1ccccc1c2-n1c2ccccc2c2cc3c4ccc5ccccc5c4n(-c4ccccc4)c3cc21 ; c1ccc(-n2c3cc4c(cc3c3c5ccccc5ccc32)c2ccccc2n4-c2ccc(-c3cccc4c3sc3ccccc34)cc2)cc1]

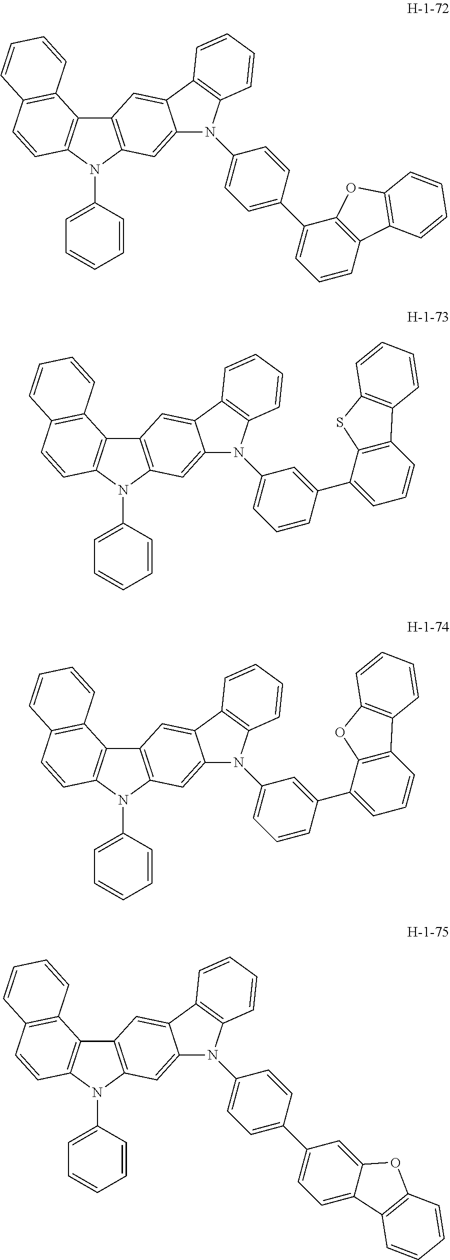 [SMILES: c1ccc(-n2c3cc4c(cc3c3c5ccccc5ccc32)c2ccccc2n4-c2ccc(-c3cccc4c3oc3ccccc34)cc2)cc1 ; c1ccc(-n2c3cc4c(cc3c3c5ccccc5ccc32)c2ccccc2n4-c2cccc(-c3cccc4c3sc3ccccc34)c2)cc1 ; c1ccc(-n2c3cc4c(cc3c3c5ccccc5ccc32)c2ccccc2n4-c2cccc(-c3cccc4c3oc3ccccc34)c2)cc1 ; c1ccc(-n2c3cc4c(cc3c3c5ccccc5ccc32)c2ccccc2n4-c2ccc(-c3ccc4c(c3)oc3ccccc34)cc2)cc1]

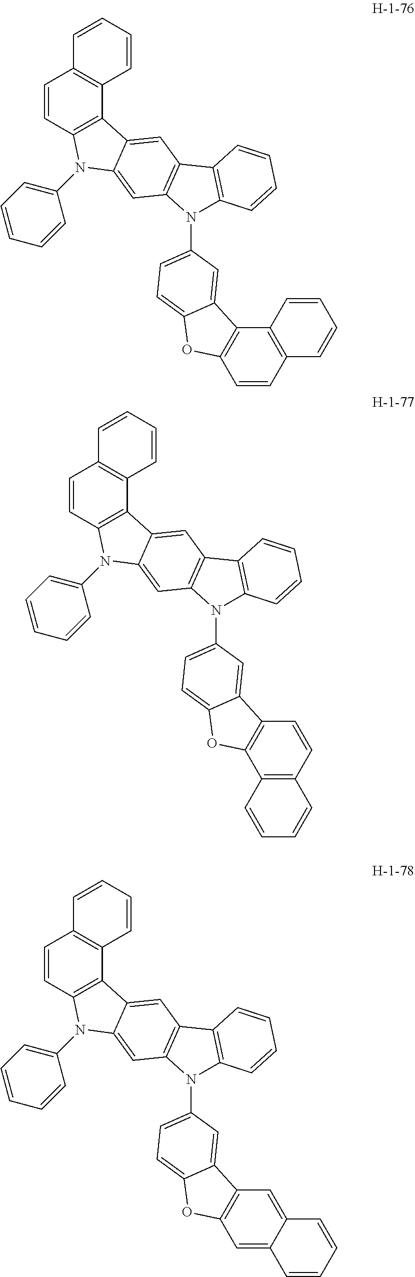 [SMILES: c1ccc(-n2c3cc4c(cc3c3c5ccccc5ccc32)c2ccccc2n4-c2ccc3oc4ccc5ccccc5c4c3c2)cc1 ; c1ccc(-n2c3cc4c(cc3c3c5ccccc5ccc32)c2ccccc2n4-c2ccc3oc4c5ccccc5ccc4c3c2)cc1 ; c1ccc(-n2c3cc4c(cc3c3c5ccccc5ccc32)c2ccccc2n4-c2ccc3oc4cc5ccccc5cc4c3c2)cc1]

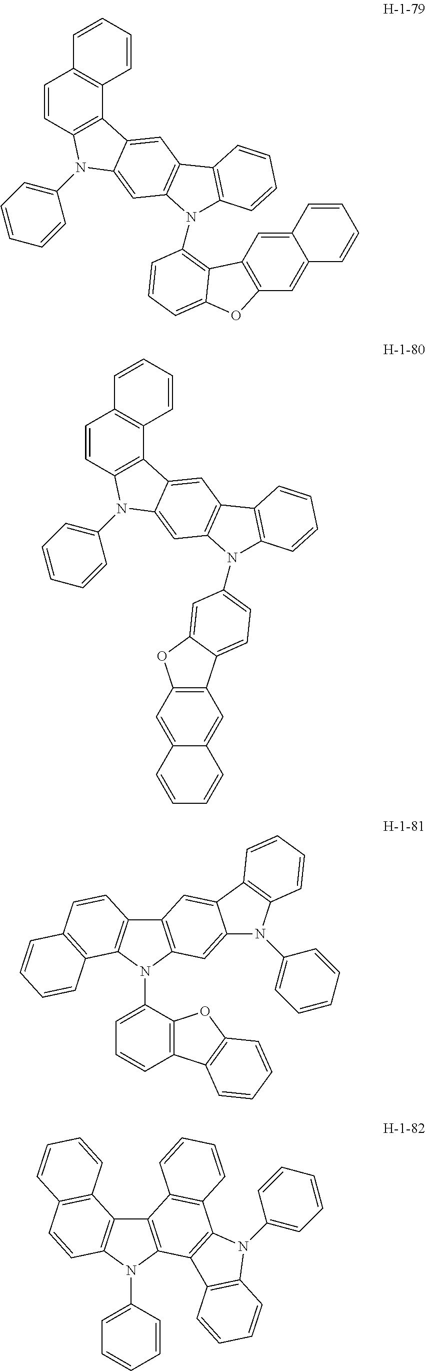 [SMILES: c1ccc(-n2c3cc4c(cc3c3c5ccccc5ccc32)c2ccccc2n4-c2cccc3oc4cc5ccccc5cc4c23)cc1 ; c1ccc(-n2c3cc4c(cc3c3c5ccccc5ccc32)c2ccccc2n4-c2ccc3c(c2)oc2cc4ccccc4cc23)cc1 ; c1ccc(-n2c3ccccc3c3cc4c5ccc6ccccc6c5n(-c5cccc6c5oc5ccccc56)c4cc32)cc1 ; c1ccc(-n2c3ccccc3c3c2c2ccccc2c2c4c5ccccc5ccc4n(-c4ccccc4)c23)cc1]

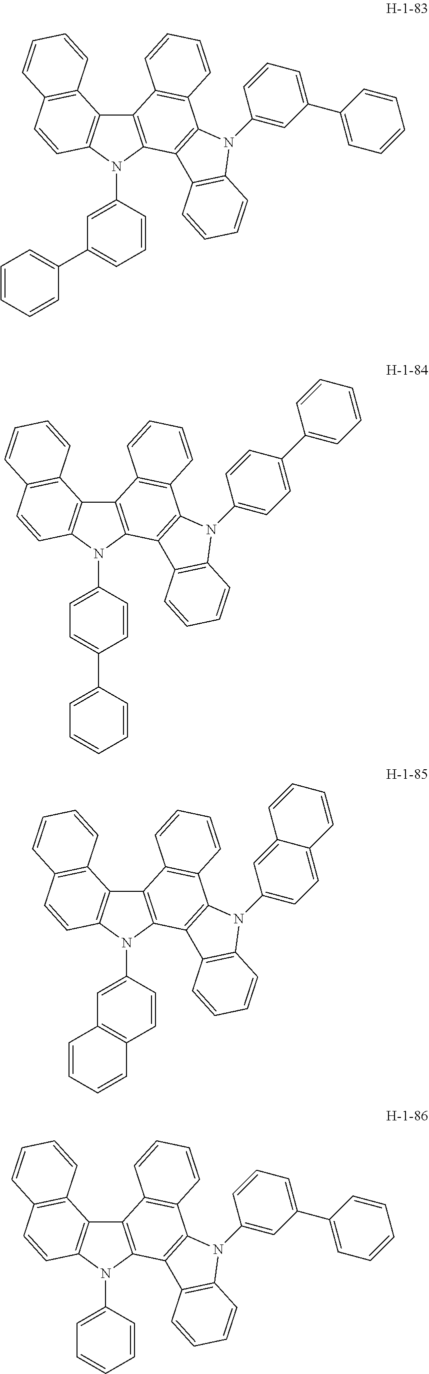 [SMILES: c1ccc(-c2cccc(-n3c4ccccc4c4c3c3ccccc3c3c5c6ccccc6ccc5n(-c5cccc(-c6ccccc6)c5)c34)c2)cc1 ; c1ccc(-c2ccc(-n3c4ccccc4c4c3c3ccccc3c3c5c6ccccc6ccc5n(-c5ccc(-c6ccccc6)cc5)c34)cc2)cc1 ; c1ccc2cc(-n3c4ccccc4c4c3c3ccccc3c3c5c6ccccc6ccc5n(-c5ccc6ccccc6c5)c34)ccc2c1 ; c1ccc(-c2cccc(-n3c4ccccc4c4c3c3ccccc3c3c5c6ccccc6ccc5n(-c5ccccc5)c34)c2)cc1]

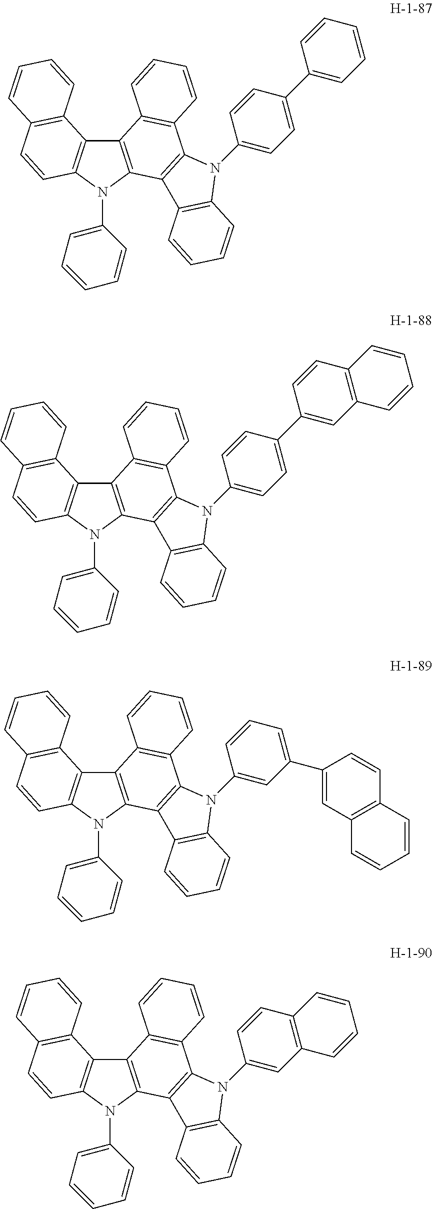 [SMILES: c1ccc(-c2ccc(-n3c4ccccc4c4c3c3ccccc3c3c5c6ccccc6ccc5n(-c5ccccc5)c34)cc2)cc1 ; c1ccc(-n2c3ccc4ccccc4c3c3c4ccccc4c4c(c5ccccc5n4-c4ccc(-c5ccc6ccccc6c5)cc4)c32)cc1 ; c1ccc(-n2c3ccc4ccccc4c3c3c4ccccc4c4c(c5ccccc5n4-c4cccc(-c5ccc6ccccc6c5)c4)c32)cc1 ; c1ccc(-n2c3ccc4ccccc4c3c3c4ccccc4c4c(c5ccccc5n4-c4ccc5ccccc5c4)c32)cc1]

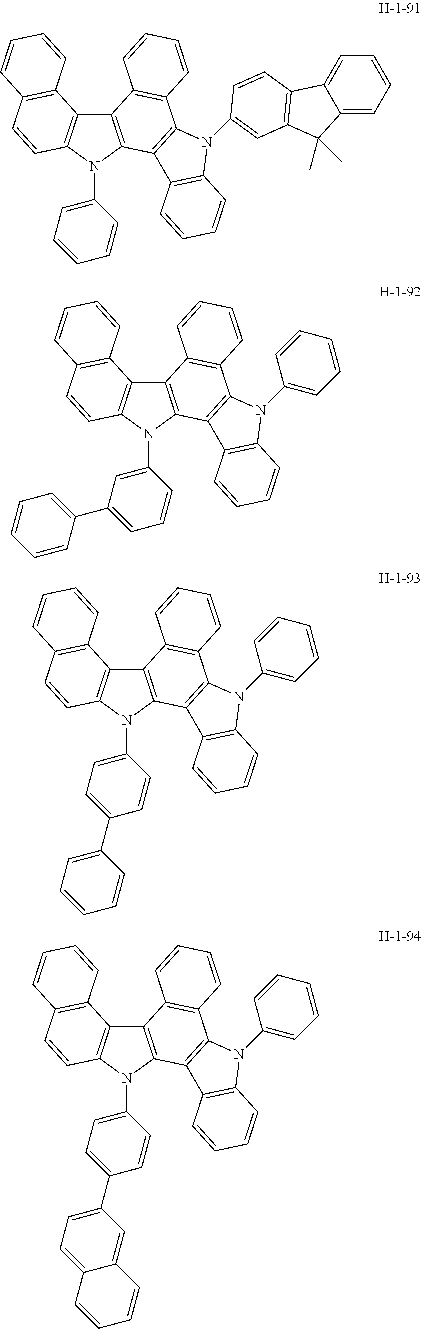

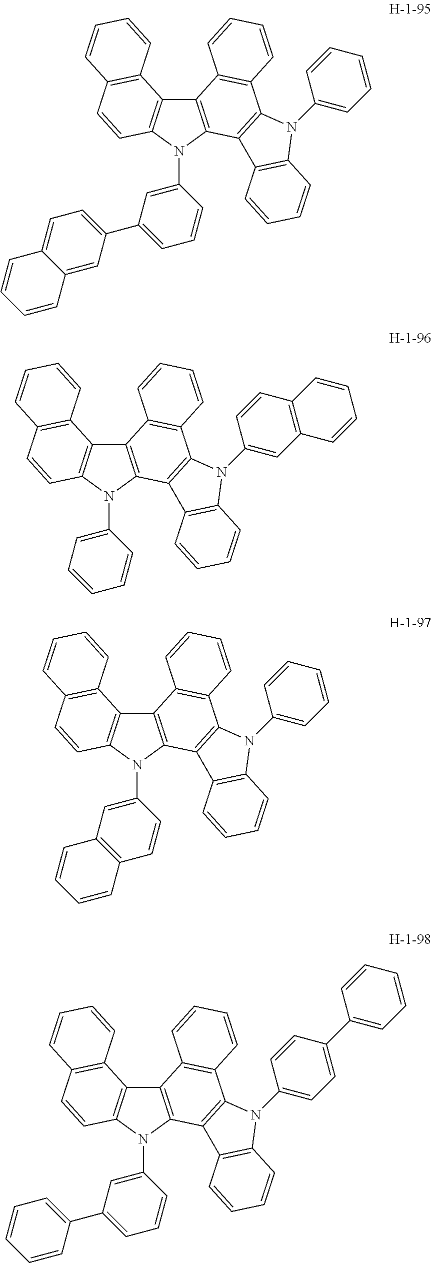

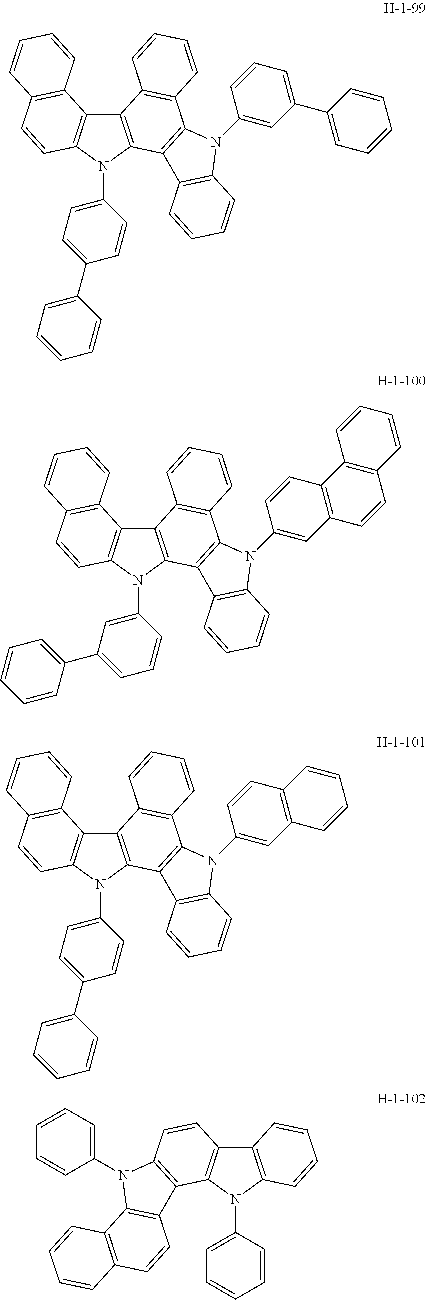

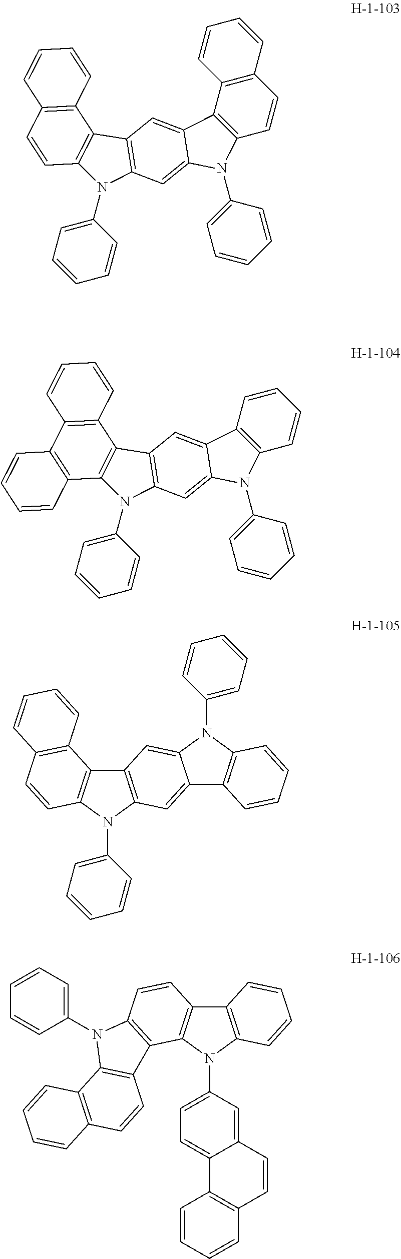 [SMILES: c1ccc(-n2c3cc4c(cc3c3c5ccccc5ccc32)c2c3ccccc3ccc2n4-c2ccccc2)cc1 ; c1ccc(-n2c3ccccc3c3cc4c5c6ccccc6c6ccccc6c5n(-c5ccccc5)c4cc32)cc1 ; c1ccc(-n2c3ccccc3c3cc4c(cc32)c2c3ccccc3ccc2n4-c2ccccc2)cc1 ; c1ccc(-n2c3ccc4c5ccccc5n(-c5ccc6c(ccc7ccccc76)c5)c4c3c3ccc4ccccc4c32)cc1]

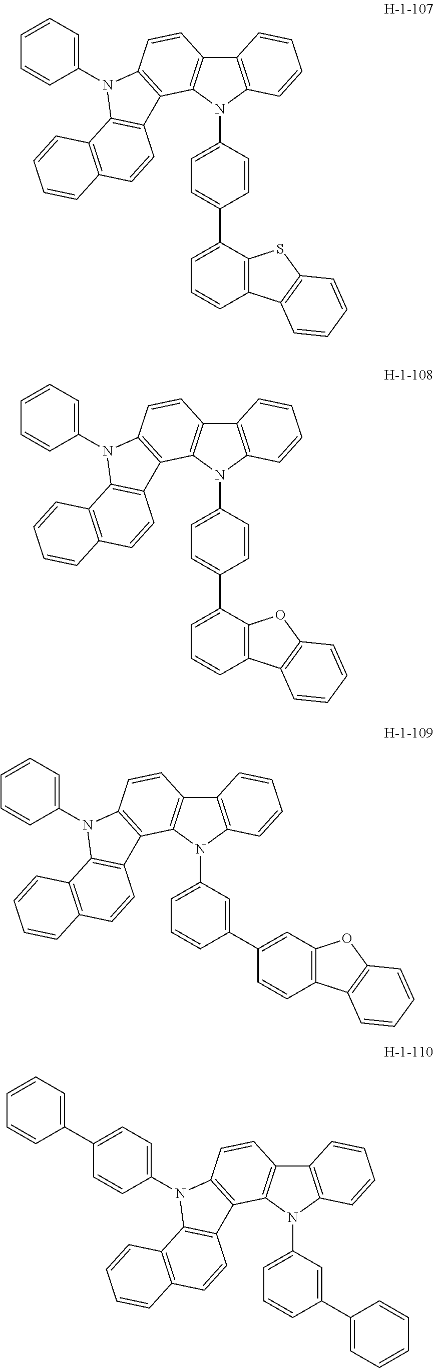 [SMILES: c1ccc(-n2c3ccc4c5ccccc5n(-c5ccc(-c6cccc7c6sc6ccccc67)cc5)c4c3c3ccc4ccccc4c32)cc1 ; c1ccc(-n2c3ccc4c5ccccc5n(-c5ccc(-c6cccc7c6oc6ccccc67)cc5)c4c3c3ccc4ccccc4c32)cc1 ; c1ccc(-n2c3ccc4c5ccccc5n(-c5cccc(-c6ccc7c(c6)oc6ccccc67)c5)c4c3c3ccc4ccccc4c32)cc1 ; c1ccc(-c2ccc(-n3c4ccc5c6ccccc6n(-c6cccc(-c7ccccc7)c6)c5c4c4ccc5ccccc5c43)cc2)cc1]

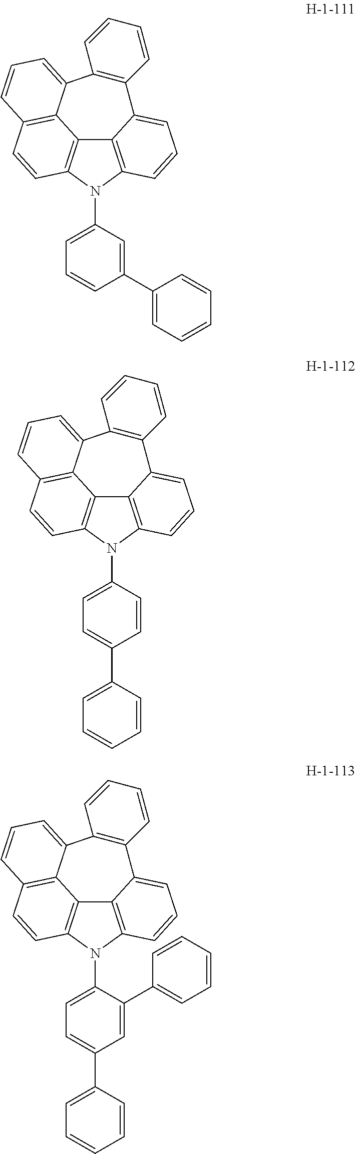

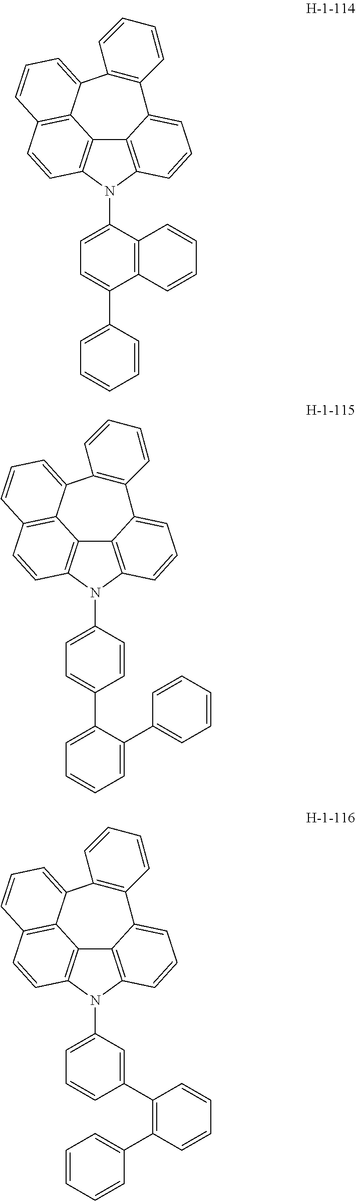

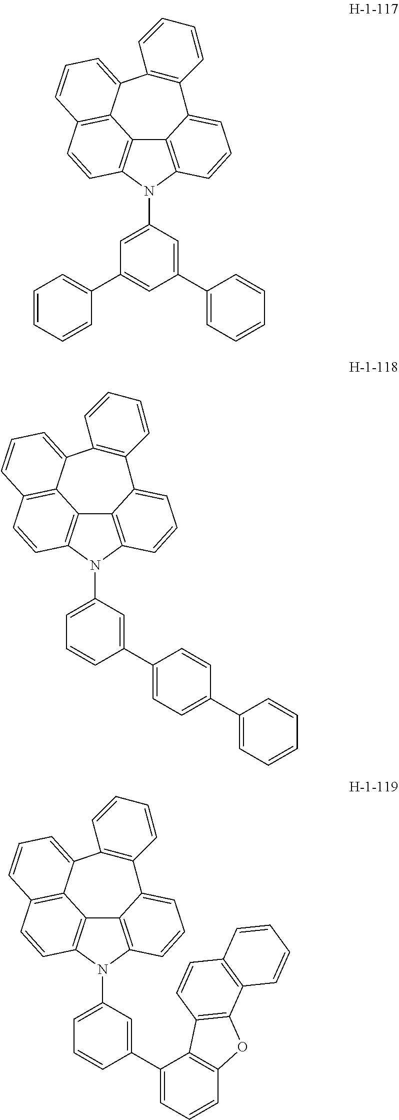 [SMILES: c1ccc(-c2cc(-c3ccccc3)cc(-n3c4cccc5c4c4c6c(cccc6ccc43)-c3ccccc3-5)c2)cc1 ; c1ccc(-c2ccc(-c3cccc(-n4c5cccc6c5c5c7c(cccc7ccc54)-c4ccccc4-6)c3)cc2)cc1 ; c1cc(-c2cccc3oc4c5ccccc5ccc4c23)cc(-n2c3cccc4c3c3c5c(cccc5ccc32)-c2ccccc2-4)c1]

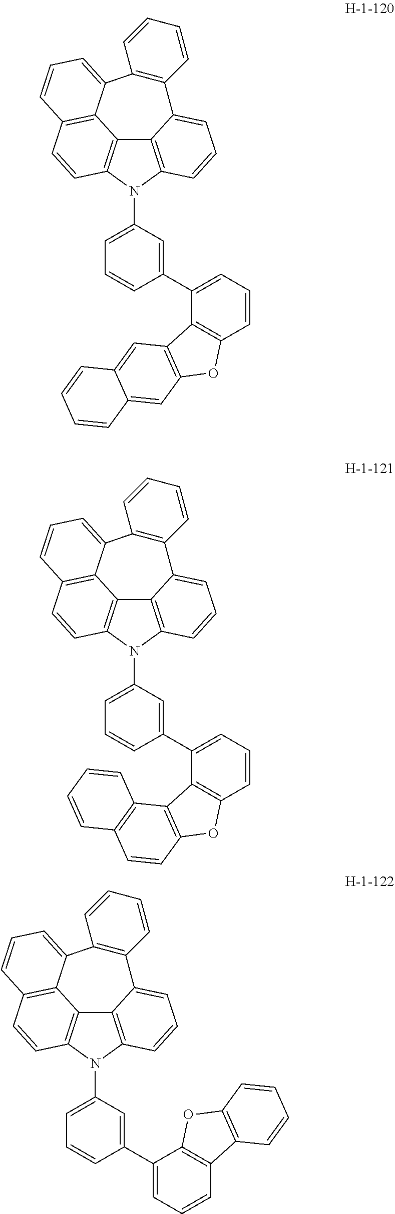

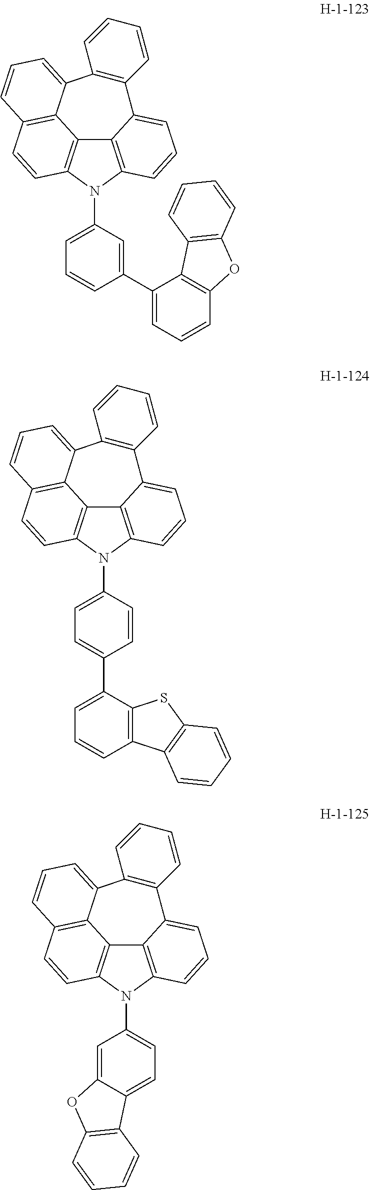 [SMILES: c1cc(-c2cccc3oc4ccccc4c23)cc(-n2c3cccc4c3c3c5c(cccc5ccc32)-c2ccccc2-4)c1 ; c1ccc2c(c1)-c1cccc3ccc4c(c13)c1c-2cccc1n4-c1ccc(-c2cccc3c2sc2ccccc23)cc1 ; c1ccc2c(c1)-c1cccc3ccc4c(c13)c1c-2cccc1n4-c1ccc2c(c1)oc1ccccc12]

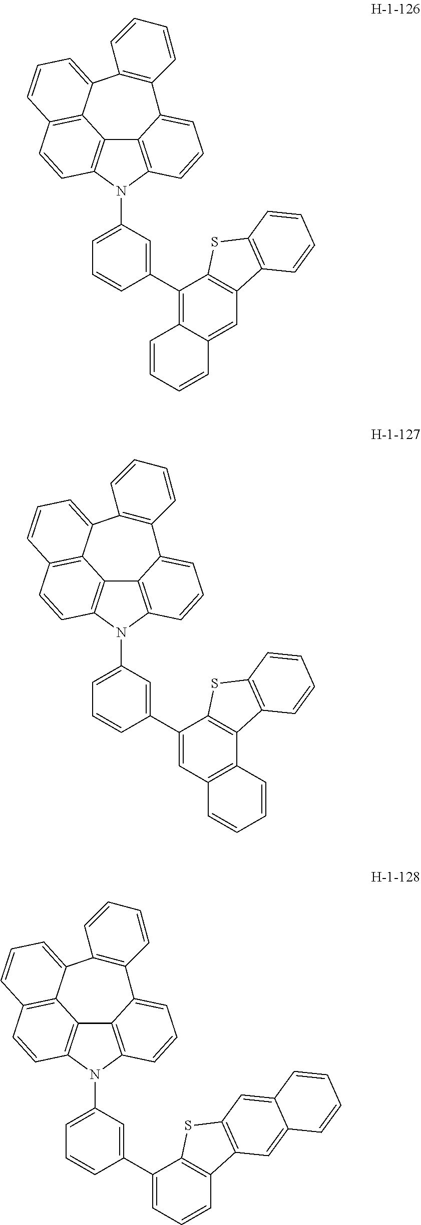 [SMILES: c1cc(-c2c3ccccc3cc3c2sc2ccccc23)cc(-n2c3cccc4c3c3c5c(cccc5ccc32)-c2ccccc2-4)c1 ; c1cc(-c2cc3ccccc3c3c2sc2ccccc23)cc(-n2c3cccc4c3c3c5c(cccc5ccc32)-c2ccccc2-4)c1 ; c1cc(-c2cccc3c2sc2cc4ccccc4cc23)cc(-n2c3cccc4c3c3c5c(cccc5ccc32)-c2ccccc2-4)c1]

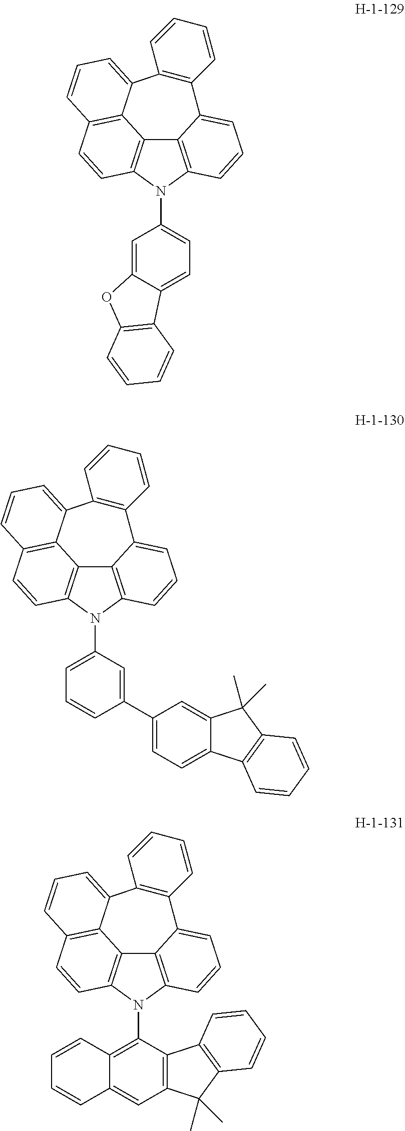 [SMILES: c1ccc2c(c1)-c1cccc3ccc4c(c13)c1c-2cccc1n4-c1ccc2c(c1)oc1ccccc12 ; CC1(C)c2ccccc2-c2ccc(-c3cccc(-n4c5cccc6c5c5c7c(cccc7ccc54)-c4ccccc4-6)c3)cc21 ; CC1(C)c2ccccc2-c2c1cc1ccccc1c2-n1c2cccc3c2c2c4c(cccc4ccc21)-c1ccccc1-3]

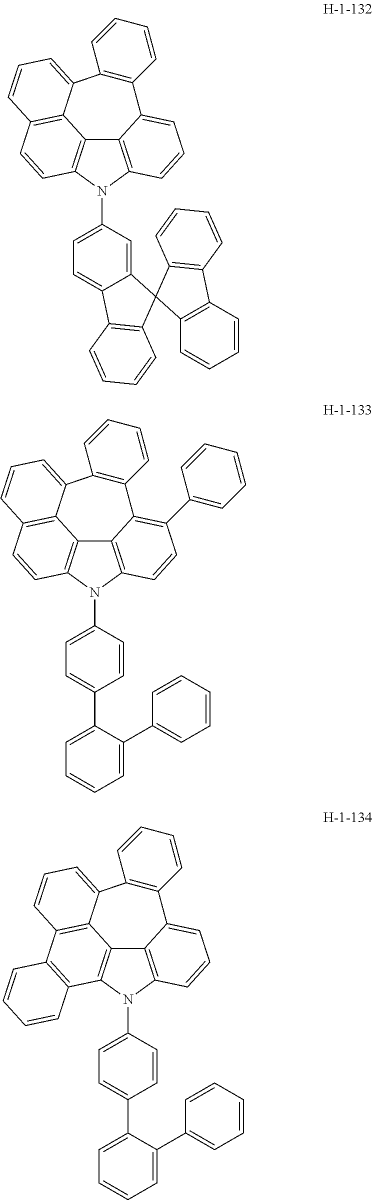

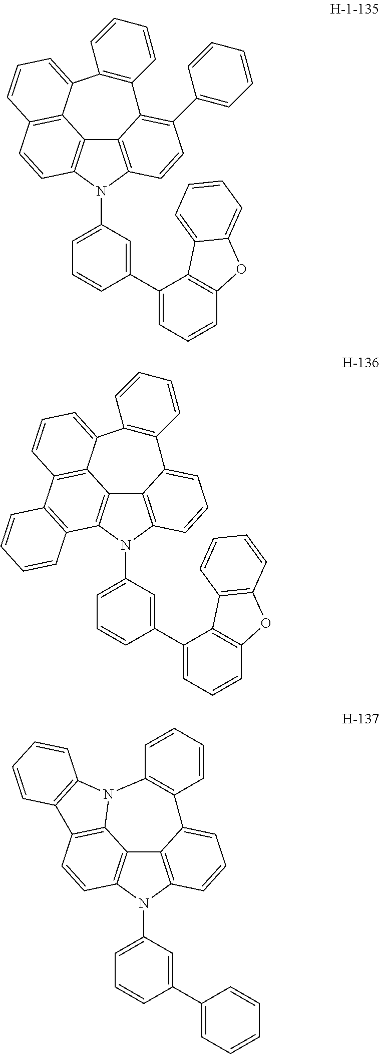

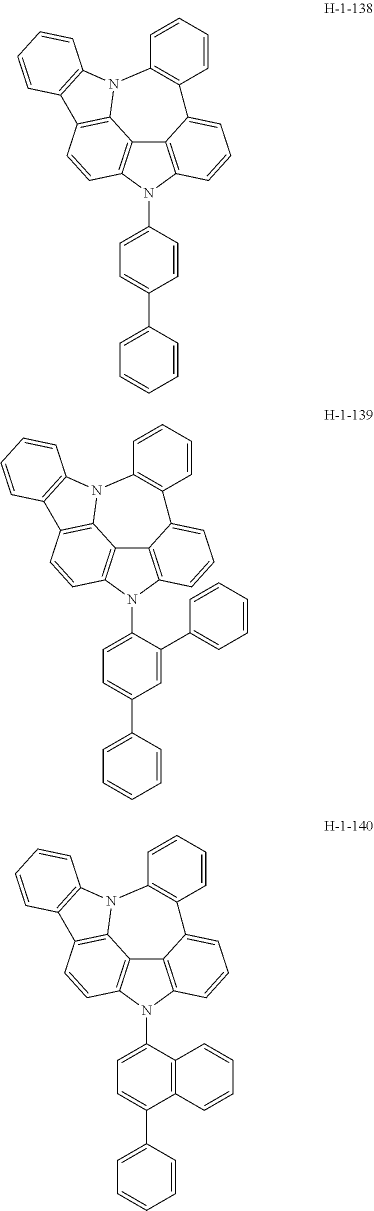

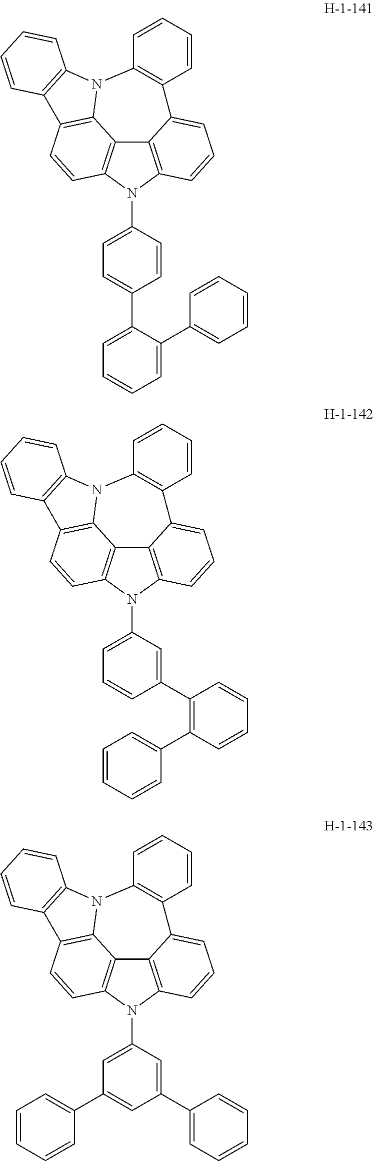

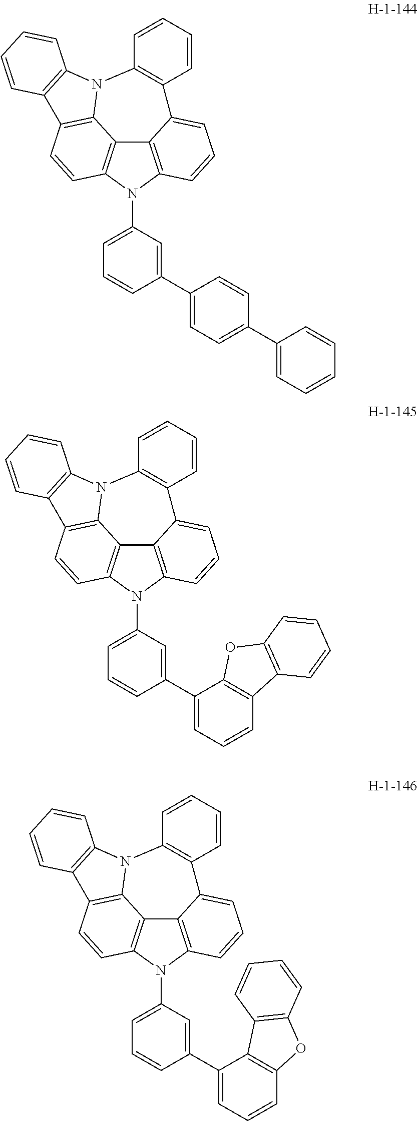 [SMILES: c1ccc(-c2ccc(-c3cccc(-n4c5cccc6c7ccccc7n7c8ccccc8c8ccc4c(c65)c87)c3)cc2)cc1 ; c1cc(-c2cccc3c2oc2ccccc23)cc(-n2c3cccc4c5ccccc5n5c6ccccc6c6ccc2c(c43)c65)c1 ; c1cc(-c2cccc3oc4ccccc4c23)cc(-n2c3cccc4c5ccccc5n5c6ccccc6c6ccc2c(c43)c65)c1]

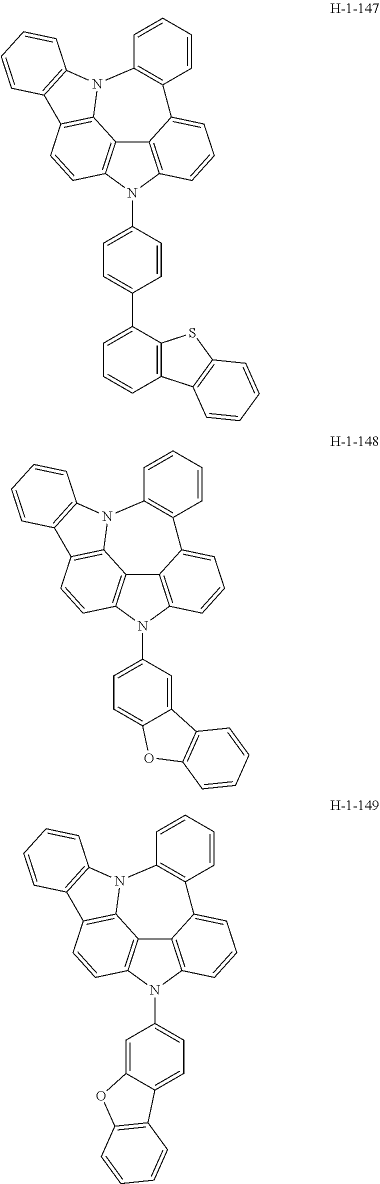

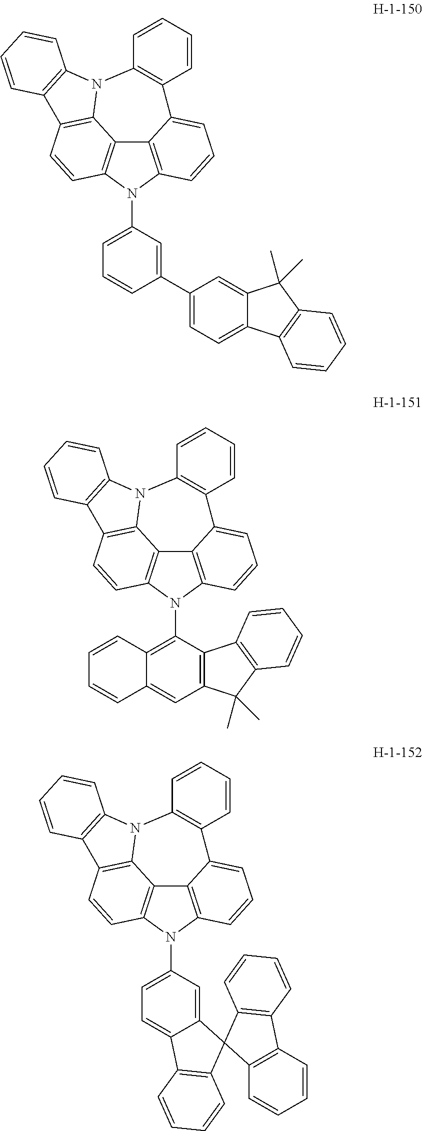

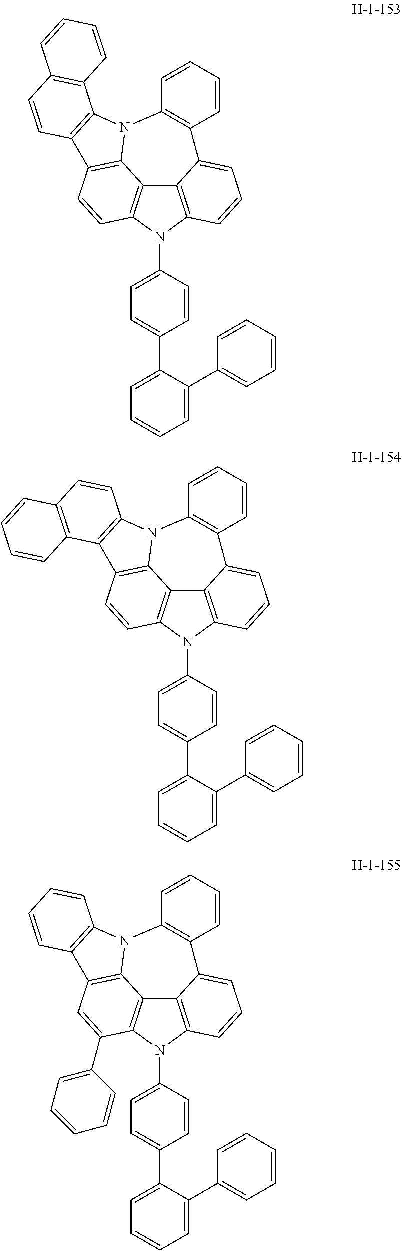

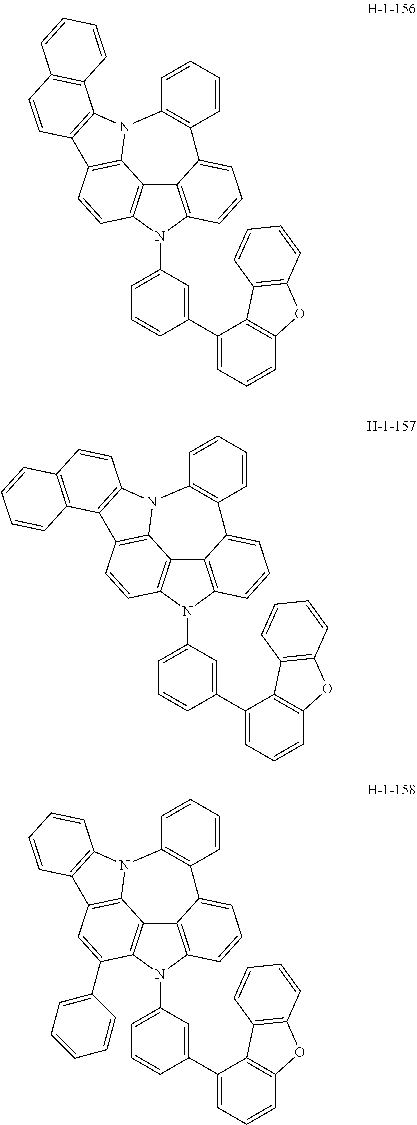 [SMILES: c1cc(-c2cccc3oc4ccccc4c23)cc(-n2c3cccc4c5ccccc5n5c6c7ccccc7ccc6c6ccc2c(c43)c65)c1 ; c1cc(-c2cccc3oc4ccccc4c23)cc(-n2c3cccc4c5ccccc5n5c6ccc7ccccc7c6c6ccc2c(c43)c65)c1 ; c1ccc(-c2cc3c4ccccc4n4c5ccccc5c5cccc6c5c(c2n6-c2cccc(-c5cccc6oc7ccccc7c56)c2)c34)cc1]

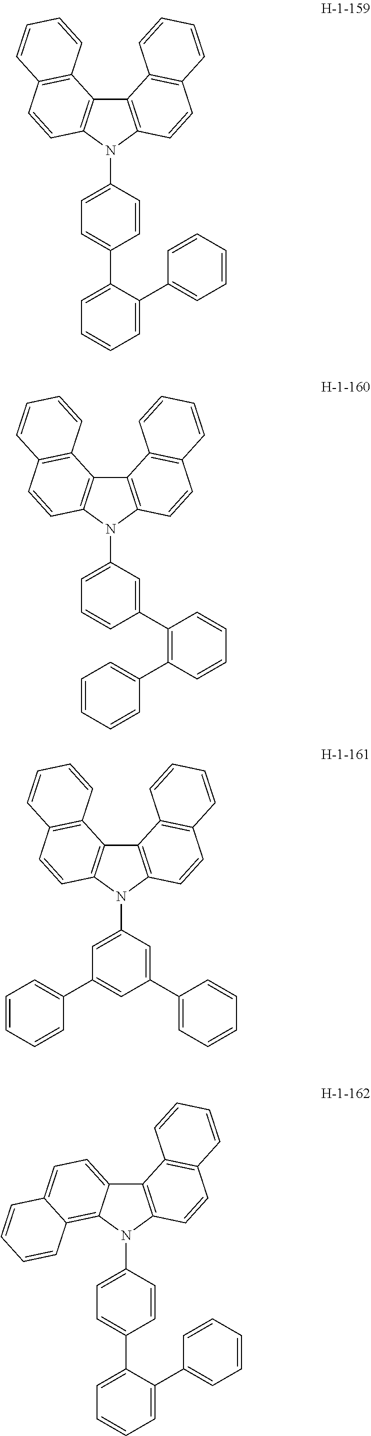 [SMILES: c1ccc(-c2ccccc2-c2ccc(-n3c4ccc5ccccc5c4c4c5ccccc5ccc43)cc2)cc1 ; c1ccc(-c2ccccc2-c2cccc(-n3c4ccc5ccccc5c4c4c5ccccc5ccc43)c2)cc1 ; c1ccc(-c2cc(-c3ccccc3)cc(-n3c4ccc5ccccc5c4c4c5ccccc5ccc43)c2)cc1 ; c1ccc(-c2ccccc2-c2ccc(-n3c4ccc5ccccc5c4c4ccc5ccccc5c43)cc2)cc1]

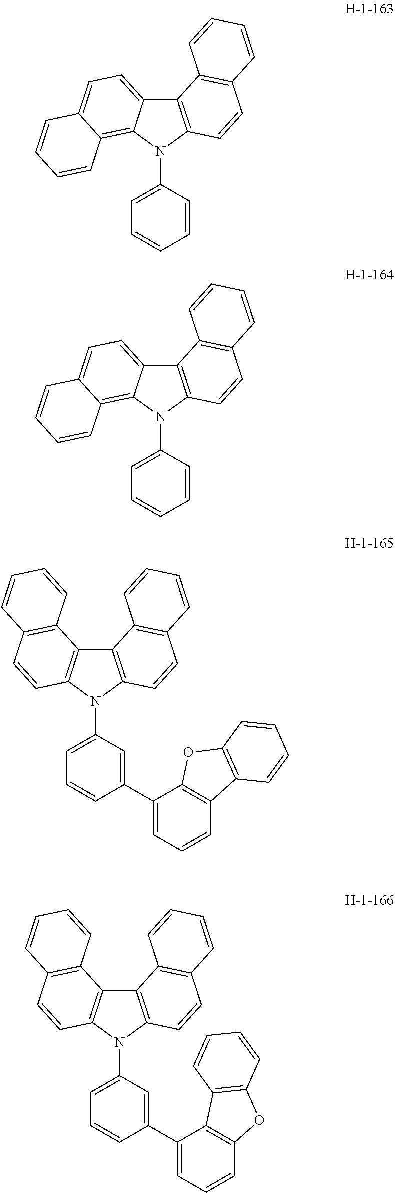 [SMILES: c1ccc(-n2c3ccc4ccccc4c3c3ccc4ccccc4c32)cc1 ; c1ccc(-n2c3ccc4ccccc4c3c3ccc4ccccc4c32)cc1 ; c1cc(-c2cccc3c2oc2ccccc23)cc(-n2c3ccc4ccccc4c3c3c4ccccc4ccc32)c1 ; c1cc(-c2cccc3oc4ccccc4c23)cc(-n2c3ccc4ccccc4c3c3c4ccccc4ccc32)c1]

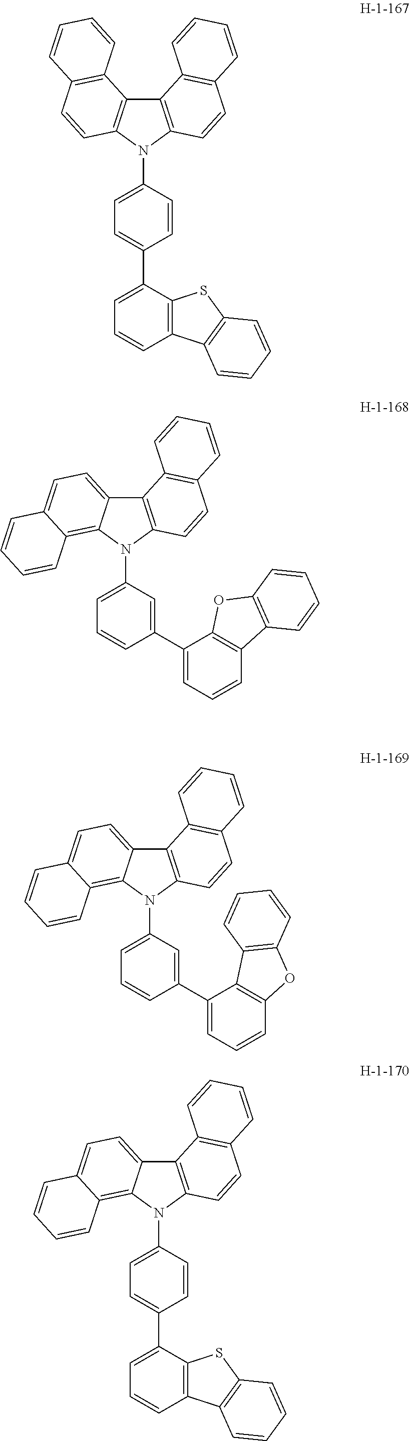

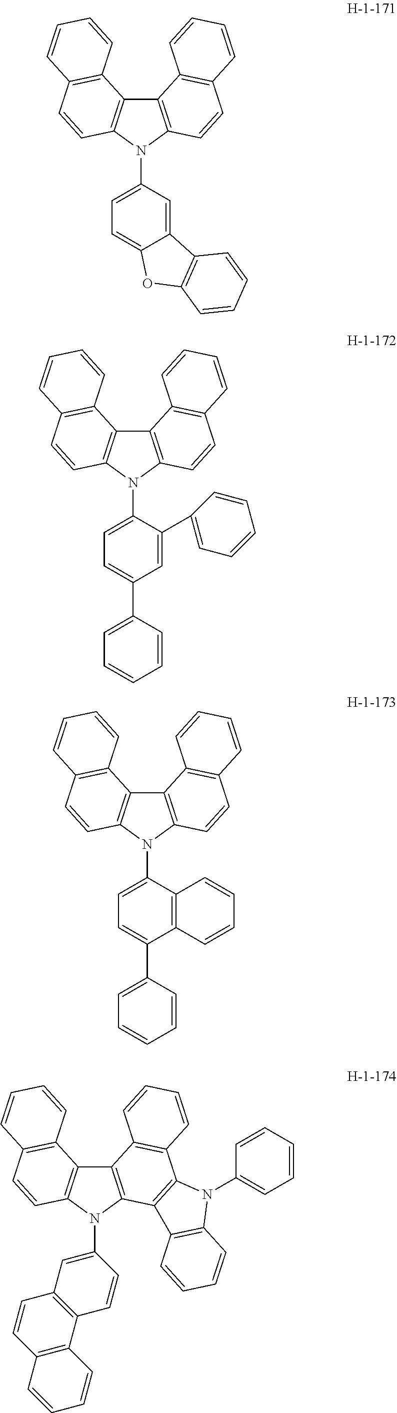

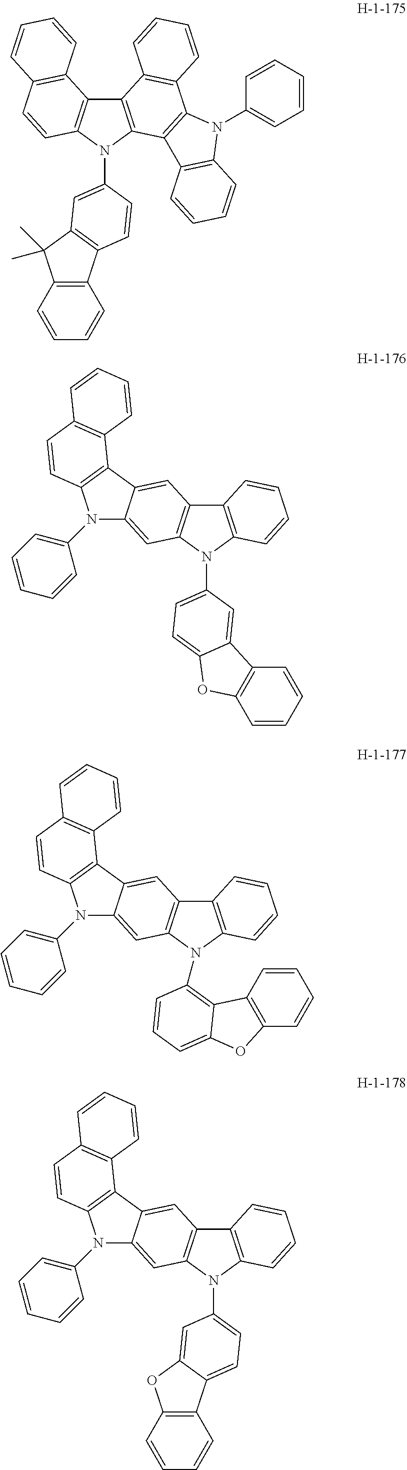

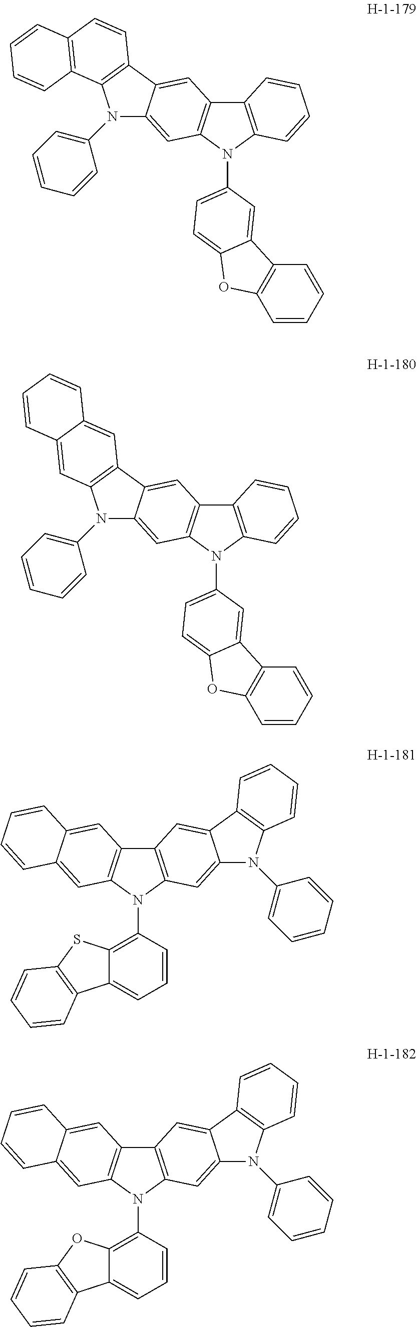

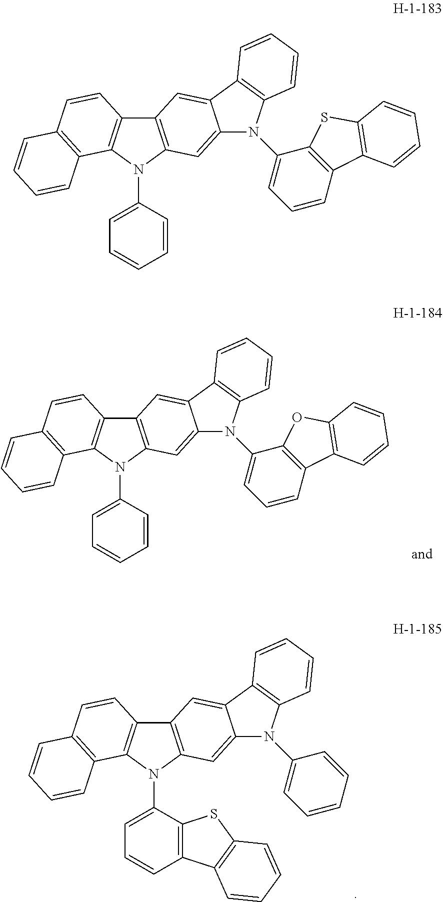 [SMILES: c1ccc(-n2c3cc4c(cc3c3ccc5ccccc5c32)c2ccccc2n4-c2cccc3c2sc2ccccc23)cc1 ; c1ccc(-n2c3cc4c(cc3c3ccc5ccccc5c32)c2ccccc2n4-c2cccc3c2oc2ccccc23)cc1 ; c1ccc(-n2c3ccccc3c3cc4c5ccc6ccccc6c5n(-c5cccc6c5sc5ccccc56)c4cc32)cc1]

6. The plurality of host materials according to claim 1, wherein the compound represented by formula 2 is at least one selected from the following compounds:

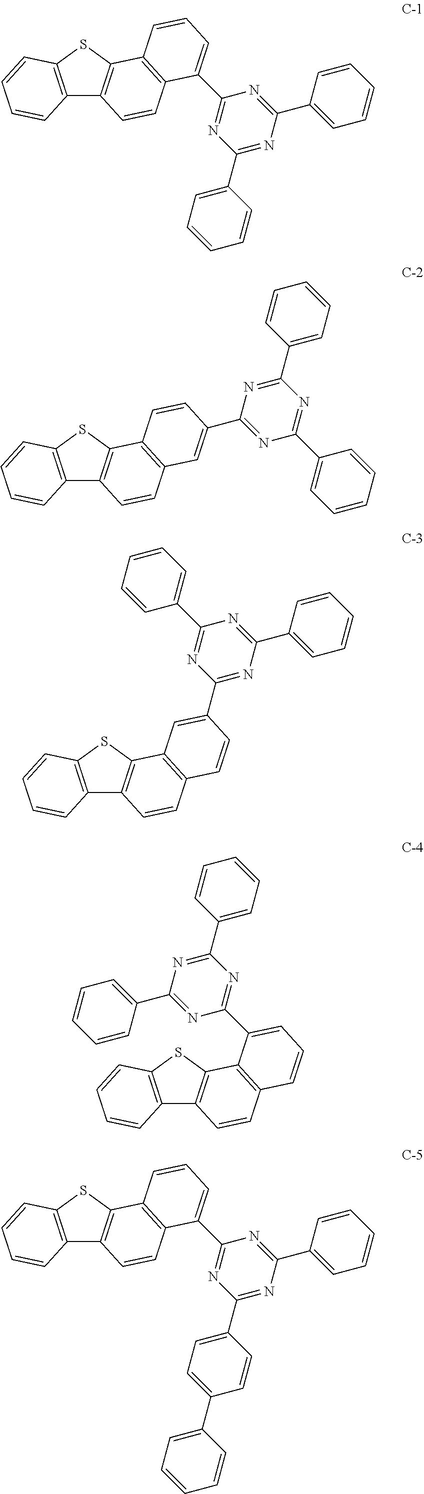

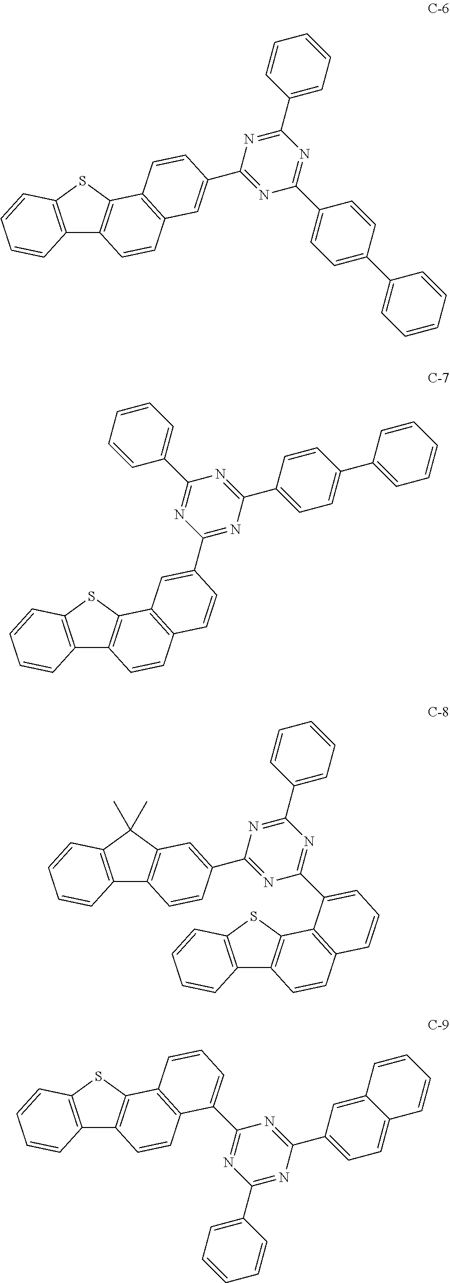 [SMILES: c1ccc(-c2ccc(-c3nc(-c4ccccc4)nc(-c4ccc5c(ccc6c7ccccc7sc56)c4)n3)cc2)cc1 ; c1ccc(-c2ccc(-c3nc(-c4ccccc4)nc(-c4ccc5ccc6c7ccccc7sc6c5c4)n3)cc2)cc1 ; CC1(C)c2ccccc2-c2ccc(-c3nc(-c4ccccc4)nc(-c4cccc5ccc6c7ccccc7sc6c45)n3)cc21 ; c1ccc(-c2nc(-c3ccc4ccccc4c3)nc(-c3cccc4c3ccc3c5ccccc5sc43)n2)cc1]

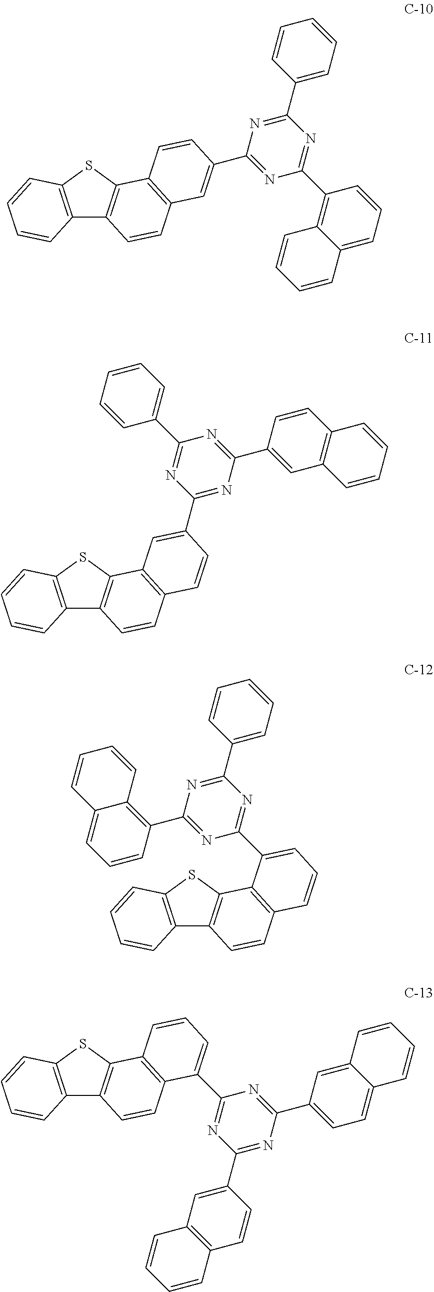 [SMILES: c1ccc(-c2nc(-c3ccc4c(ccc5c6ccccc6sc45)c3)nc(-c3cccc4ccccc34)n2)cc1 ; c1ccc(-c2nc(-c3ccc4ccccc4c3)nc(-c3ccc4ccc5c6ccccc6sc5c4c3)n2)cc1 ; c1ccc(-c2nc(-c3cccc4ccccc34)nc(-c3cccc4ccc5c6ccccc6sc5c34)n2)cc1 ; c1ccc2cc(-c3nc(-c4ccc5ccccc5c4)nc(-c4cccc5c4ccc4c6ccccc6sc54)n3)ccc2c1]

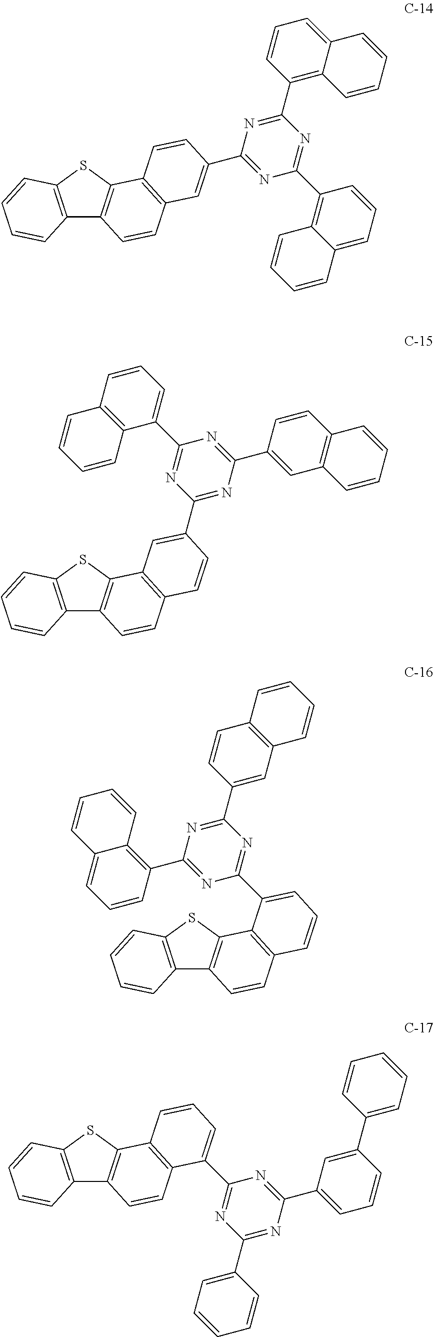 [SMILES: c1ccc2c(-c3nc(-c4ccc5c(ccc6c7ccccc7sc56)c4)nc(-c4cccc5ccccc45)n3)cccc2c1 ; c1ccc2cc(-c3nc(-c4ccc5ccc6c7ccccc7sc6c5c4)nc(-c4cccc5ccccc45)n3)ccc2c1 ; c1ccc2cc(-c3nc(-c4cccc5ccccc45)nc(-c4cccc5ccc6c7ccccc7sc6c45)n3)ccc2c1 ; c1ccc(-c2cccc(-c3nc(-c4ccccc4)nc(-c4cccc5c4ccc4c6ccccc6sc54)n3)c2)cc1]

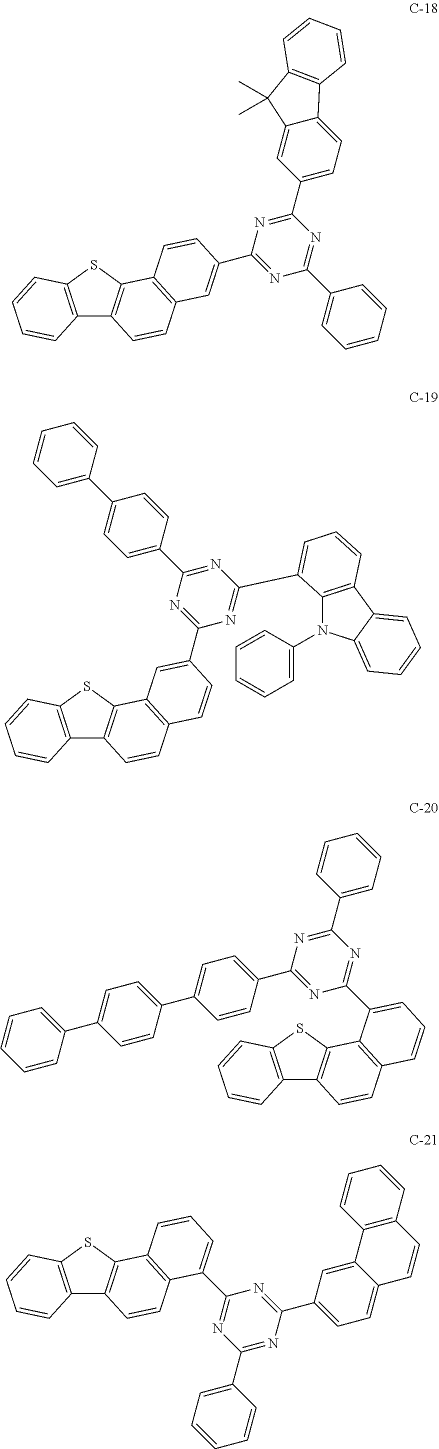 [SMILES: CC1(C)c2ccccc2-c2ccc(-c3nc(-c4ccccc4)nc(-c4ccc5c(ccc6c7ccccc7sc56)c4)n3)cc21 ; c1ccc(-c2ccc(-c3nc(-c4ccc5ccc6c7ccccc7sc6c5c4)nc(-c4cccc5c6ccccc6n(-c6ccccc6)c45)n3)cc2)cc1 ; c1ccc(-c2ccc(-c3ccc(-c4nc(-c5ccccc5)nc(-c5cccc6ccc7c8ccccc8sc7c56)n4)cc3)cc2)cc1 ; c1ccc(-c2nc(-c3ccc4ccc5ccccc5c4c3)nc(-c3cccc4c3ccc3c5ccccc5sc43)n2)cc1]

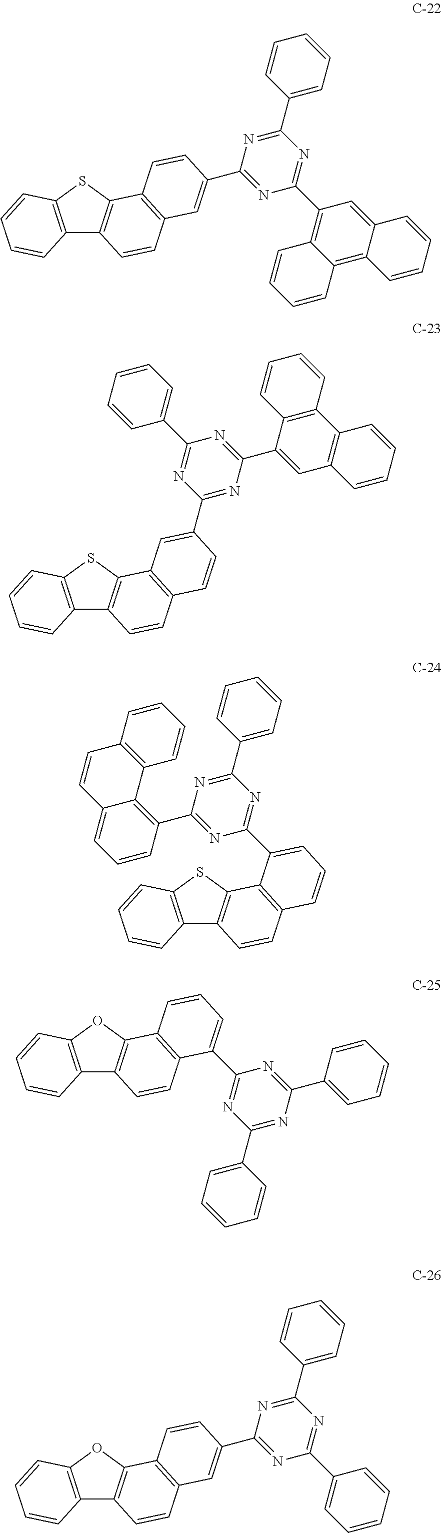 [SMILES: c1ccc(-c2nc(-c3ccc4c(ccc5c6ccccc6sc45)c3)nc(-c3cc4ccccc4c4ccccc34)n2)cc1 ; c1ccc(-c2nc(-c3ccc4ccc5c6ccccc6sc5c4c3)nc(-c3cc4ccccc4c4ccccc34)n2)cc1 ; c1ccc(-c2nc(-c3cccc4ccc5ccccc5c34)nc(-c3cccc4ccc5c6ccccc6sc5c34)n2)cc1 ; c1ccc(-c2nc(-c3ccccc3)nc(-c3cccc4c3ccc3c5ccccc5oc43)n2)cc1 ; c1ccc(-c2nc(-c3ccccc3)nc(-c3ccc4c(ccc5c6ccccc6oc45)c3)n2)cc1]

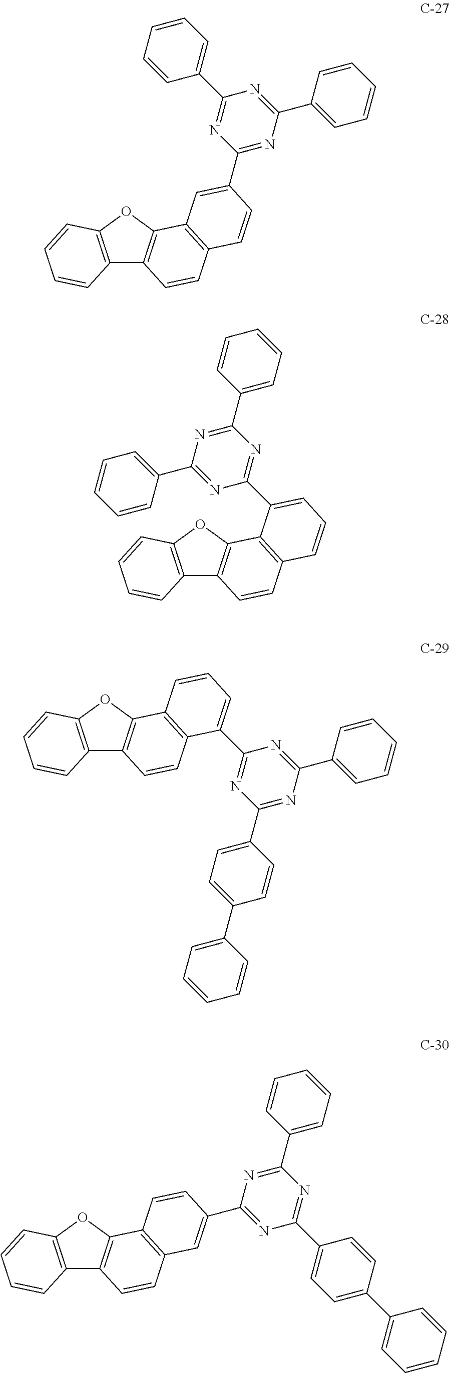 [SMILES: c1ccc(-c2nc(-c3ccccc3)nc(-c3ccc4ccc5c6ccccc6oc5c4c3)n2)cc1 ; c1ccc(-c2nc(-c3ccccc3)nc(-c3cccc4ccc5c6ccccc6oc5c34)n2)cc1 ; c1ccc(-c2ccc(-c3nc(-c4ccccc4)nc(-c4cccc5c4ccc4c6ccccc6oc54)n3)cc2)cc1 ; c1ccc(-c2ccc(-c3nc(-c4ccccc4)nc(-c4ccc5c(ccc6c7ccccc7oc56)c4)n3)cc2)cc1]

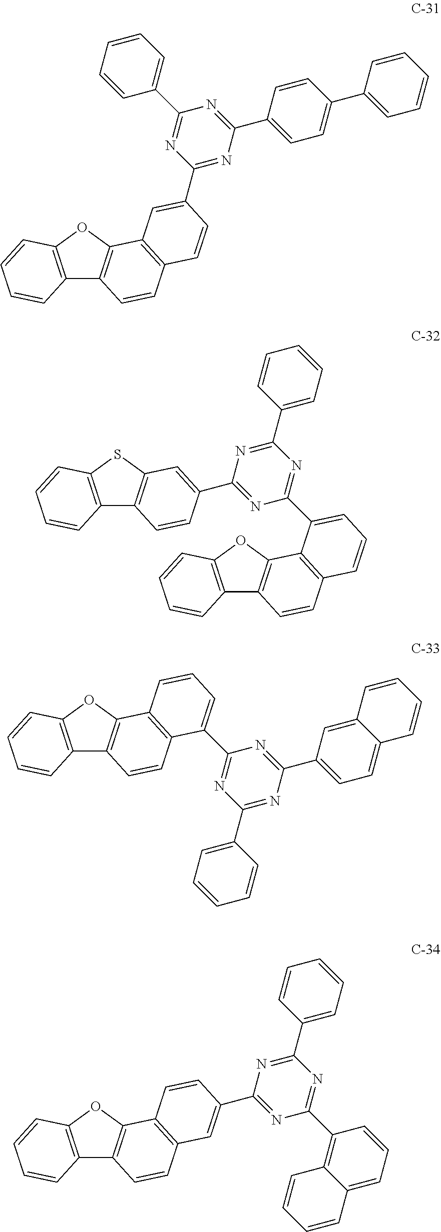 [SMILES: c1ccc(-c2ccc(-c3nc(-c4ccccc4)nc(-c4ccc5ccc6c7ccccc7oc6c5c4)n3)cc2)cc1 ; c1ccc(-c2nc(-c3ccc4c(c3)sc3ccccc34)nc(-c3cccc4ccc5c6ccccc6oc5c34)n2)cc1 ; c1ccc(-c2nc(-c3ccc4ccccc4c3)nc(-c3cccc4c3ccc3c5ccccc5oc43)n2)cc1 ; c1ccc(-c2nc(-c3ccc4c(ccc5c6ccccc6oc45)c3)nc(-c3cccc4ccccc34)n2)cc1]

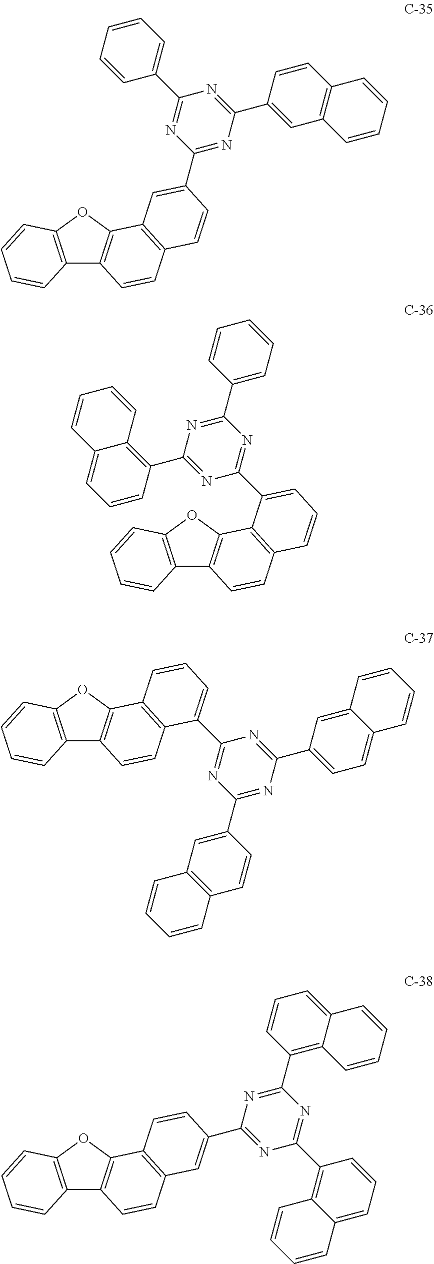 [SMILES: c1ccc(-c2nc(-c3ccc4ccccc4c3)nc(-c3ccc4ccc5c6ccccc6oc5c4c3)n2)cc1 ; c1ccc(-c2nc(-c3cccc4ccccc34)nc(-c3cccc4ccc5c6ccccc6oc5c34)n2)cc1 ; c1ccc2cc(-c3nc(-c4ccc5ccccc5c4)nc(-c4cccc5c4ccc4c6ccccc6oc54)n3)ccc2c1 ; c1ccc2c(-c3nc(-c4ccc5c(ccc6c7ccccc7oc56)c4)nc(-c4cccc5ccccc45)n3)cccc2c1]

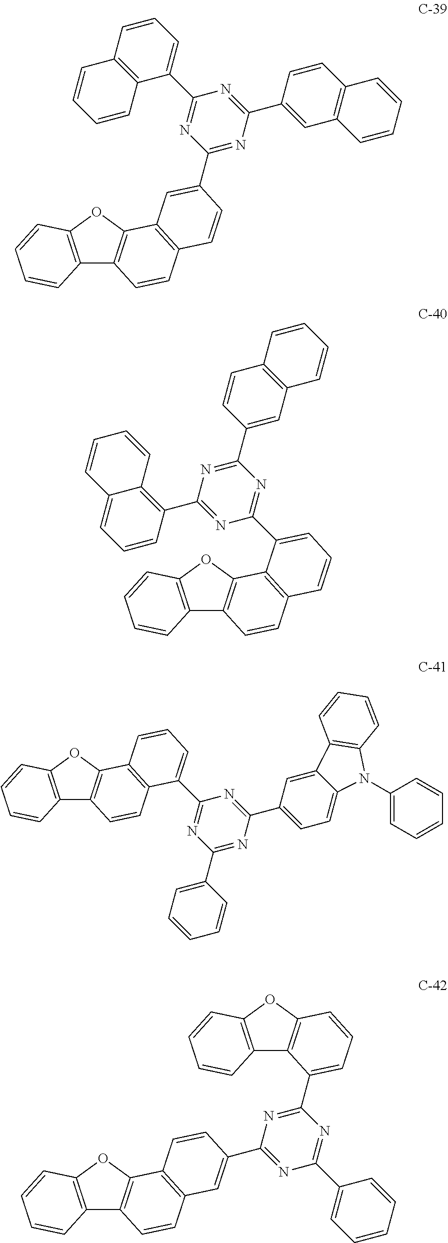 [SMILES: c1ccc2cc(-c3nc(-c4ccc5ccc6c7ccccc7oc6c5c4)nc(-c4cccc5ccccc45)n3)ccc2c1 ; c1ccc2cc(-c3nc(-c4cccc5ccccc45)nc(-c4cccc5ccc6c7ccccc7oc6c45)n3)ccc2c1 ; c1ccc(-c2nc(-c3ccc4c(c3)c3ccccc3n4-c3ccccc3)nc(-c3cccc4c3ccc3c5ccccc5oc43)n2)cc1 ; c1ccc(-c2nc(-c3ccc4c(ccc5c6ccccc6oc45)c3)nc(-c3cccc4oc5ccccc5c34)n2)cc1]

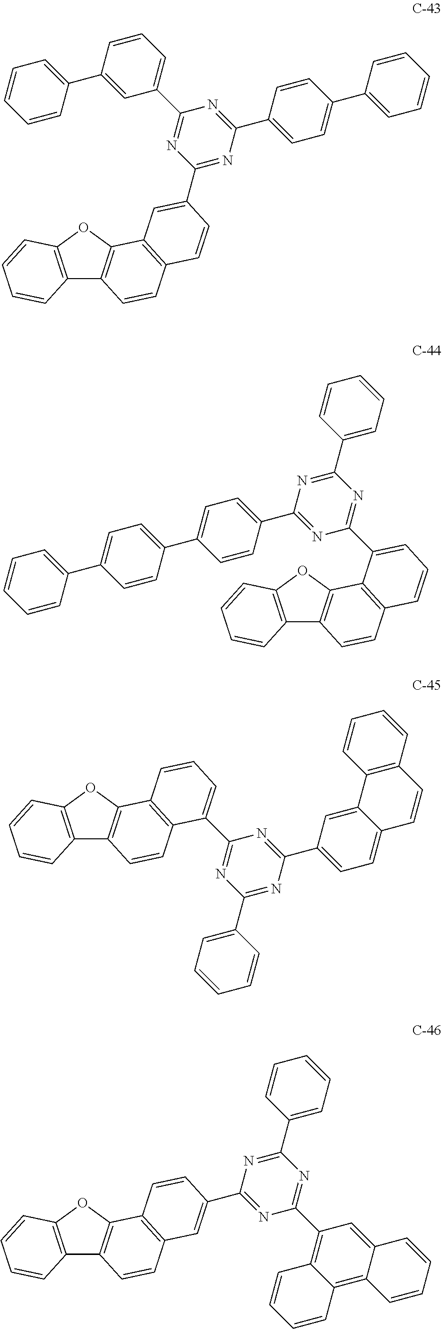 [SMILES: c1ccc(-c2ccc(-c3nc(-c4cccc(-c5ccccc5)c4)nc(-c4ccc5ccc6c7ccccc7oc6c5c4)n3)cc2)cc1 ; c1ccc(-c2ccc(-c3ccc(-c4nc(-c5ccccc5)nc(-c5cccc6ccc7c8ccccc8oc7c56)n4)cc3)cc2)cc1 ; c1ccc(-c2nc(-c3ccc4ccc5ccccc5c4c3)nc(-c3cccc4c3ccc3c5ccccc5oc43)n2)cc1 ; c1ccc(-c2nc(-c3ccc4c(ccc5c6ccccc6oc45)c3)nc(-c3cc4ccccc4c4ccccc34)n2)cc1]

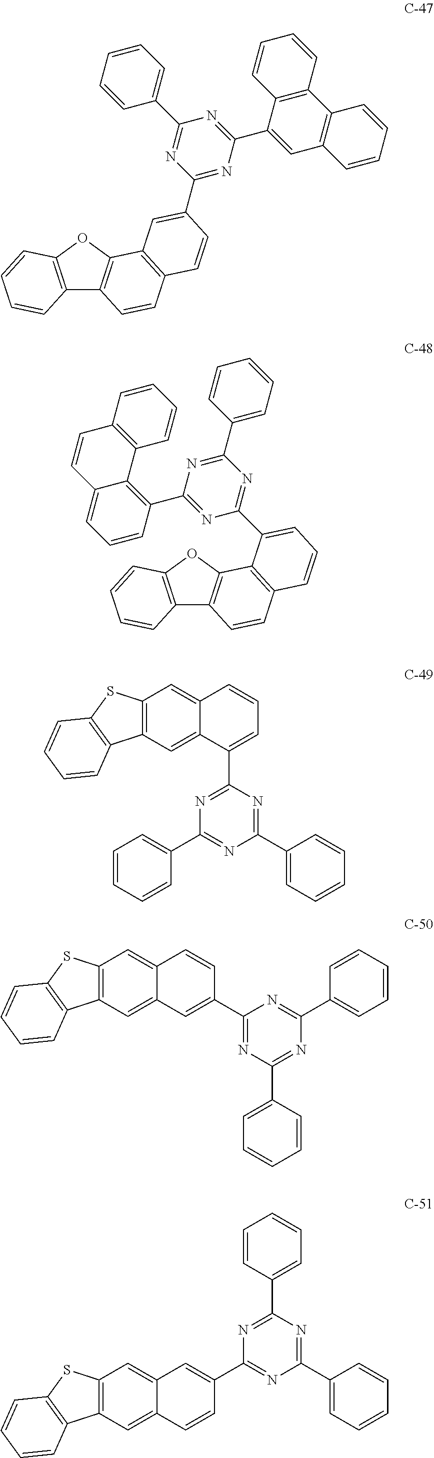 [SMILES: c1ccc(-c2nc(-c3ccc4ccc5c6ccccc6oc5c4c3)nc(-c3cc4ccccc4c4ccccc34)n2)cc1 ; c1ccc(-c2nc(-c3cccc4ccc5ccccc5c34)nc(-c3cccc4ccc5c6ccccc6oc5c34)n2)cc1 ; c1ccc(-c2nc(-c3ccccc3)nc(-c3cccc4cc5sc6ccccc6c5cc34)n2)cc1 ; c1ccc(-c2nc(-c3ccccc3)nc(-c3ccc4cc5sc6ccccc6c5cc4c3)n2)cc1 ; c1ccc(-c2nc(-c3ccccc3)nc(-c3ccc4cc5c(cc4c3)sc3ccccc35)n2)cc1]

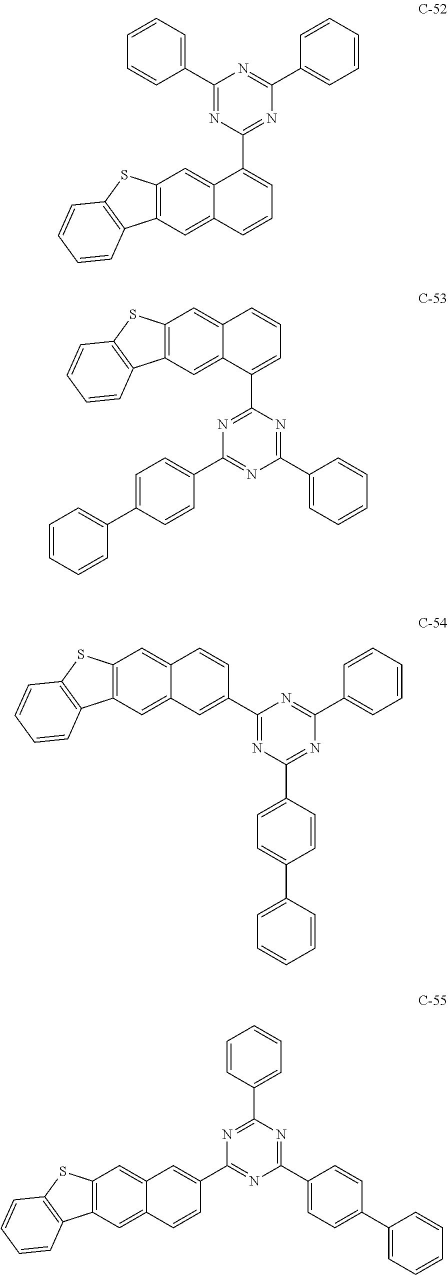 [SMILES: c1ccc(-c2nc(-c3ccccc3)nc(-c3cccc4cc5c(cc34)sc3ccccc35)n2)cc1 ; c1ccc(-c2ccc(-c3nc(-c4ccccc4)nc(-c4cccc5cc6sc7ccccc7c6cc45)n3)cc2)cc1 ; c1ccc(-c2ccc(-c3nc(-c4ccccc4)nc(-c4ccc5cc6sc7ccccc7c6cc5c4)n3)cc2)cc1 ; c1ccc(-c2ccc(-c3nc(-c4ccccc4)nc(-c4ccc5cc6c(cc5c4)sc4ccccc46)n3)cc2)cc1]

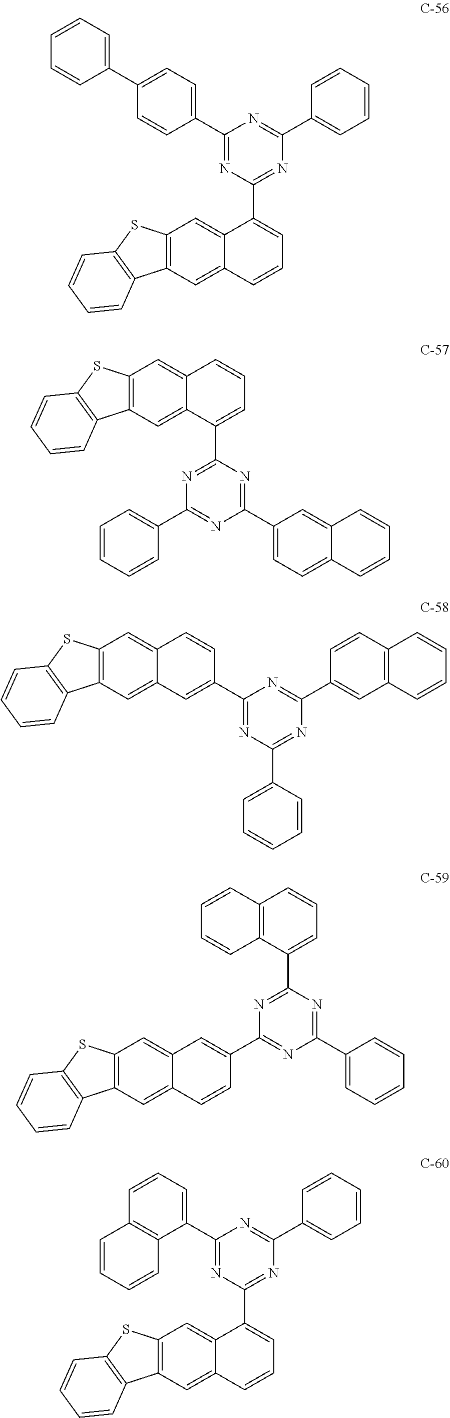

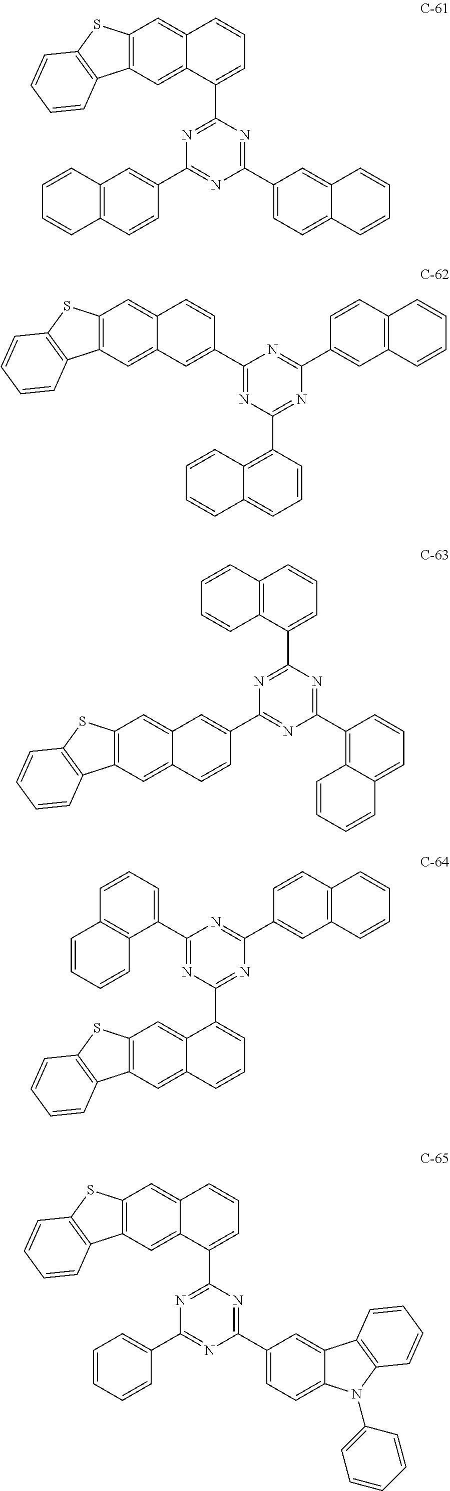

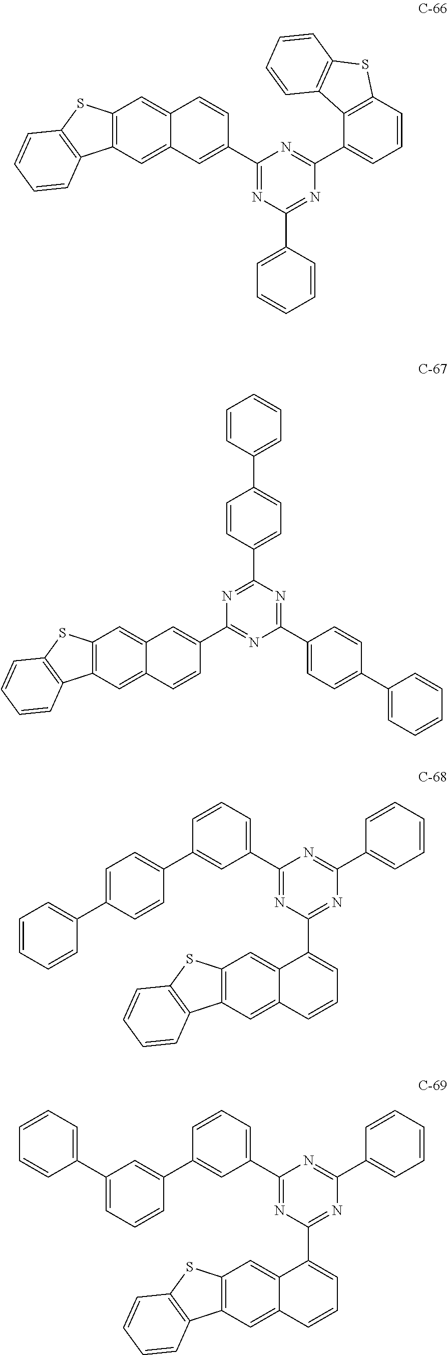 [SMILES: c1ccc(-c2nc(-c3ccc4cc5sc6ccccc6c5cc4c3)nc(-c3cccc4sc5ccccc5c34)n2)cc1 ; c1ccc(-c2ccc(-c3nc(-c4ccc(-c5ccccc5)cc4)nc(-c4ccc5cc6c(cc5c4)sc4ccccc46)n3)cc2)cc1 ; c1ccc(-c2ccc(-c3cccc(-c4nc(-c5ccccc5)nc(-c5cccc6cc7c(cc56)sc5ccccc57)n4)c3)cc2)cc1 ; c1ccc(-c2cccc(-c3cccc(-c4nc(-c5ccccc5)nc(-c5cccc6cc7c(cc56)sc5ccccc57)n4)c3)c2)cc1]

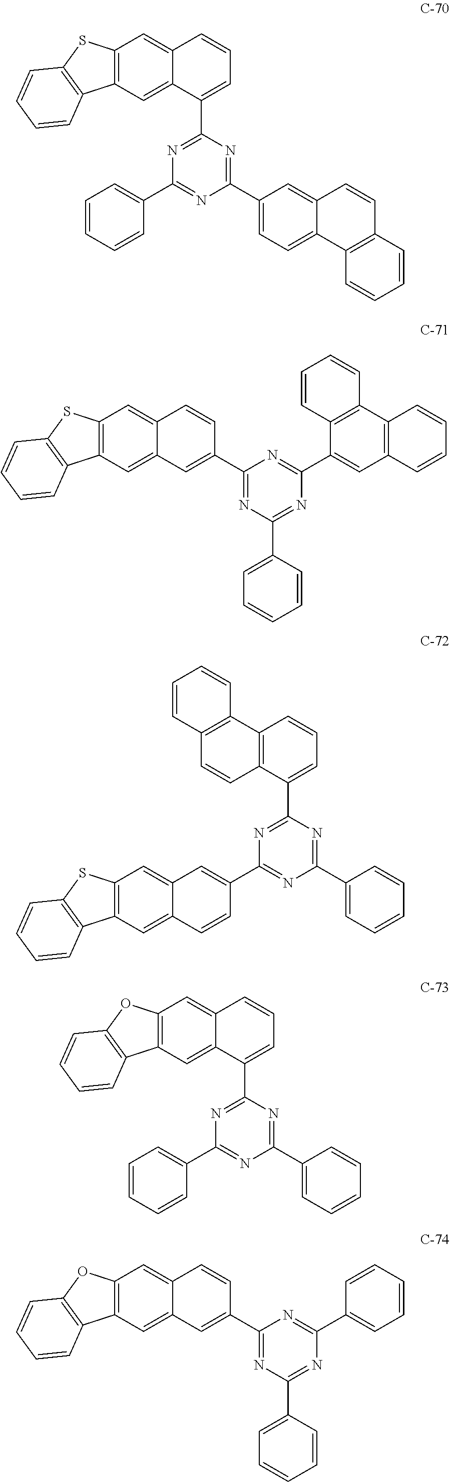 [SMILES: c1ccc(-c2nc(-c3ccc4c(ccc5ccccc54)c3)nc(-c3cccc4cc5sc6ccccc6c5cc34)n2)cc1 ; c1ccc(-c2nc(-c3ccc4cc5sc6ccccc6c5cc4c3)nc(-c3cc4ccccc4c4ccccc34)n2)cc1 ; c1ccc(-c2nc(-c3ccc4cc5c(cc4c3)sc3ccccc35)nc(-c3cccc4c3ccc3ccccc34)n2)cc1 ; c1ccc(-c2nc(-c3ccccc3)nc(-c3cccc4cc5oc6ccccc6c5cc34)n2)cc1 ; c1ccc(-c2nc(-c3ccccc3)nc(-c3ccc4cc5oc6ccccc6c5cc4c3)n2)cc1]

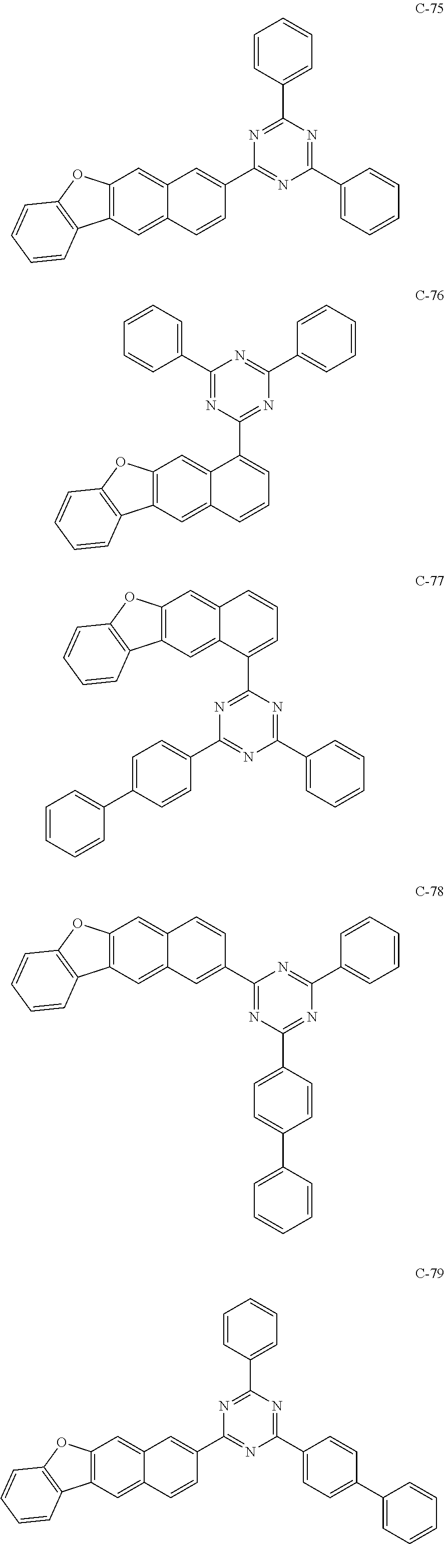 [SMILES: c1ccc(-c2nc(-c3ccccc3)nc(-c3ccc4cc5c(cc4c3)oc3ccccc35)n2)cc1 ; c1ccc(-c2nc(-c3ccccc3)nc(-c3cccc4cc5c(cc34)oc3ccccc35)n2)cc1 ; c1ccc(-c2ccc(-c3nc(-c4ccccc4)nc(-c4cccc5cc6oc7ccccc7c6cc45)n3)cc2)cc1 ; c1ccc(-c2ccc(-c3nc(-c4ccccc4)nc(-c4ccc5cc6oc7ccccc7c6cc5c4)n3)cc2)cc1 ; c1ccc(-c2ccc(-c3nc(-c4ccccc4)nc(-c4ccc5cc6c(cc5c4)oc4ccccc46)n3)cc2)cc1]

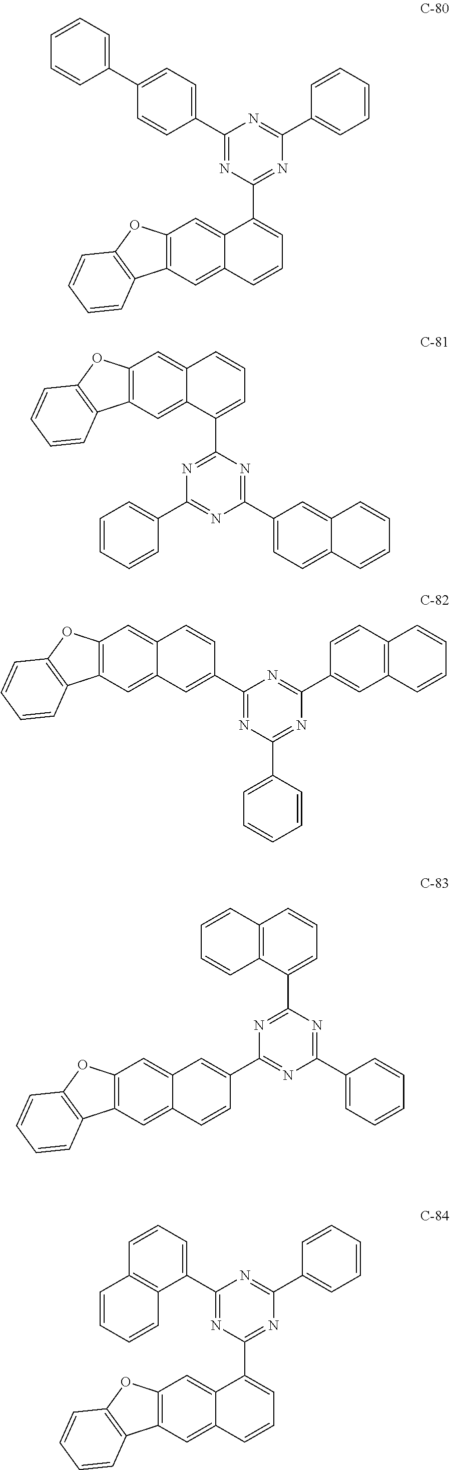

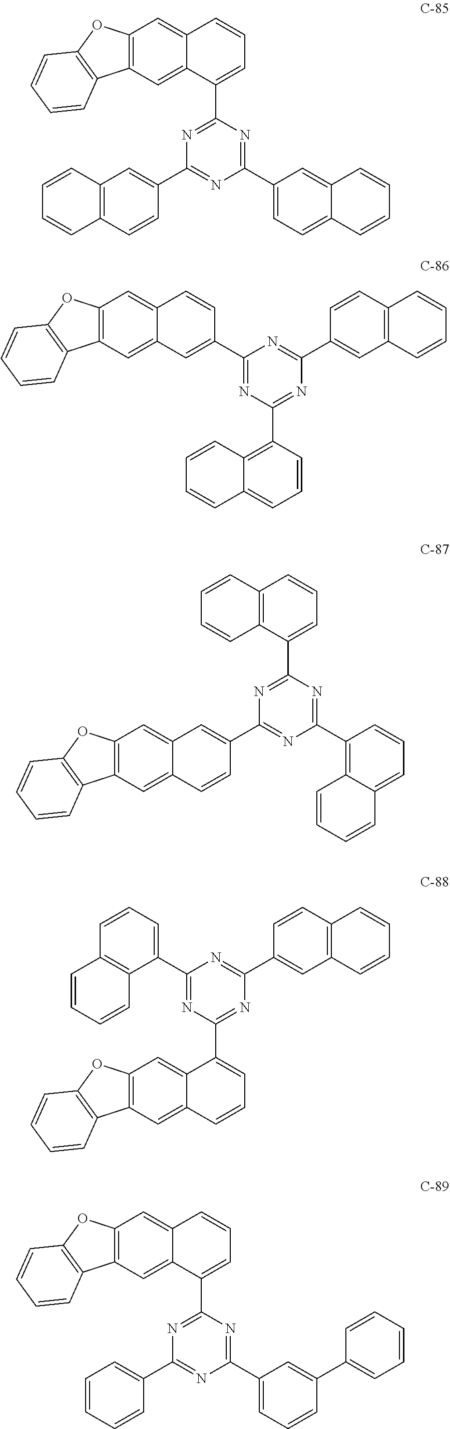 [SMILES: c1ccc2cc(-c3nc(-c4ccc5ccccc5c4)nc(-c4cccc5cc6oc7ccccc7c6cc45)n3)ccc2c1 ; c1ccc2cc(-c3nc(-c4ccc5cc6oc7ccccc7c6cc5c4)nc(-c4cccc5ccccc45)n3)ccc2c1 ; c1ccc2c(-c3nc(-c4ccc5cc6c(cc5c4)oc4ccccc46)nc(-c4cccc5ccccc45)n3)cccc2c1 ; c1ccc2cc(-c3nc(-c4cccc5ccccc45)nc(-c4cccc5cc6c(cc45)oc4ccccc46)n3)ccc2c1 ; c1ccc(-c2cccc(-c3nc(-c4ccccc4)nc(-c4cccc5cc6oc7ccccc7c6cc45)n3)c2)cc1]

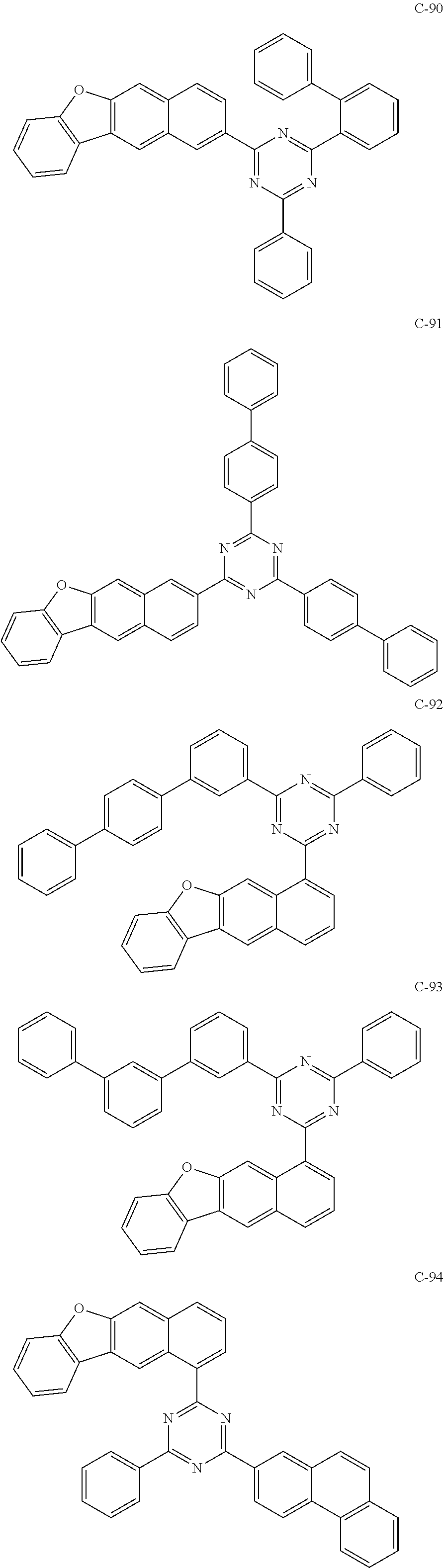 [SMILES: c1ccc(-c2nc(-c3ccc4cc5oc6ccccc6c5cc4c3)nc(-c3ccccc3-c3ccccc3)n2)cc1 ; c1ccc(-c2ccc(-c3nc(-c4ccc(-c5ccccc5)cc4)nc(-c4ccc5cc6c(cc5c4)oc4ccccc46)n3)cc2)cc1 ; c1ccc(-c2ccc(-c3cccc(-c4nc(-c5ccccc5)nc(-c5cccc6cc7c(cc56)oc5ccccc57)n4)c3)cc2)cc1 ; c1ccc(-c2cccc(-c3cccc(-c4nc(-c5ccccc5)nc(-c5cccc6cc7c(cc56)oc5ccccc57)n4)c3)c2)cc1 ; c1ccc(-c2nc(-c3ccc4c(ccc5ccccc54)c3)nc(-c3cccc4cc5oc6ccccc6c5cc34)n2)cc1]

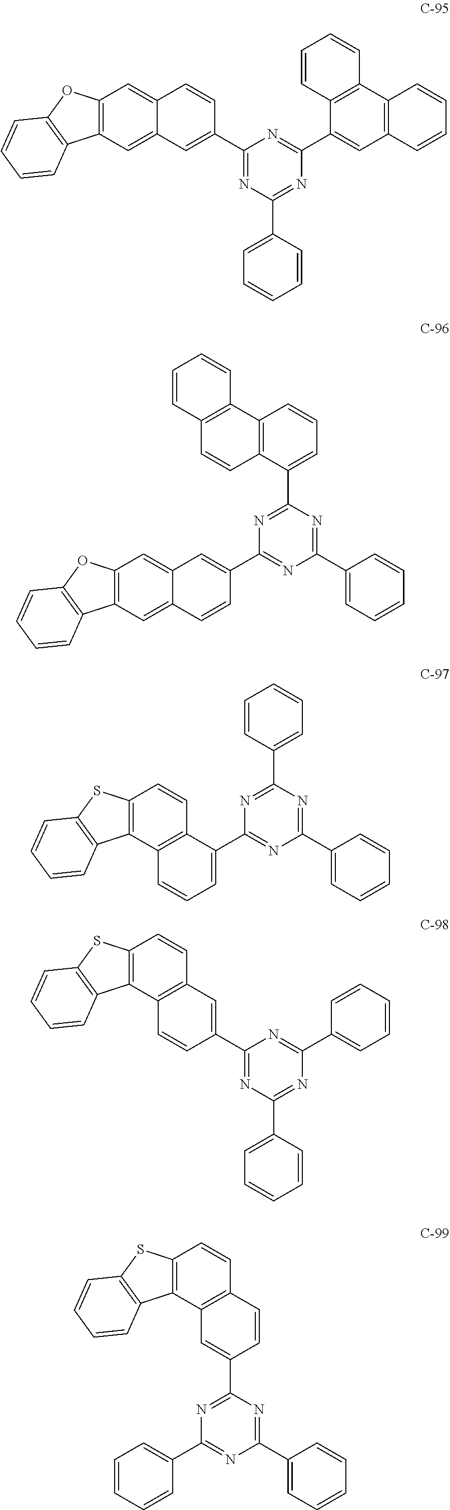

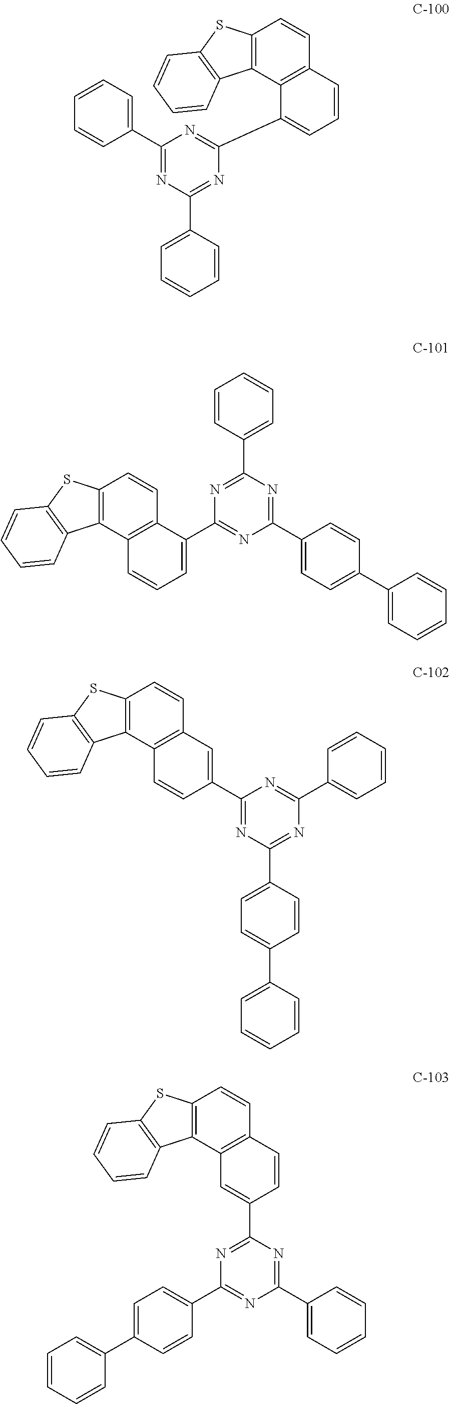 [SMILES: c1ccc(-c2nc(-c3ccccc3)nc(-c3cccc4ccc5sc6ccccc6c5c34)n2)cc1 ; c1ccc(-c2ccc(-c3nc(-c4ccccc4)nc(-c4cccc5c4ccc4sc6ccccc6c45)n3)cc2)cc1 ; c1ccc(-c2ccc(-c3nc(-c4ccccc4)nc(-c4ccc5c(ccc6sc7ccccc7c65)c4)n3)cc2)cc1 ; c1ccc(-c2ccc(-c3nc(-c4ccccc4)nc(-c4ccc5ccc6sc7ccccc7c6c5c4)n3)cc2)cc1]

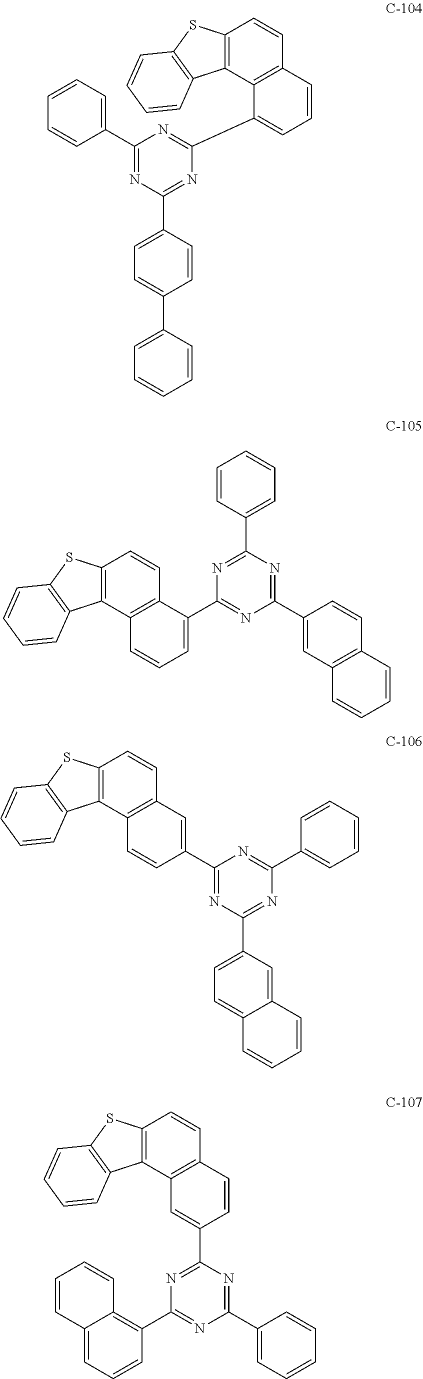 [SMILES: c1ccc(-c2ccc(-c3nc(-c4ccccc4)nc(-c4cccc5ccc6sc7ccccc7c6c45)n3)cc2)cc1 ; c1ccc(-c2nc(-c3ccc4ccccc4c3)nc(-c3cccc4c3ccc3sc5ccccc5c34)n2)cc1 ; c1ccc(-c2nc(-c3ccc4ccccc4c3)nc(-c3ccc4c(ccc5sc6ccccc6c54)c3)n2)cc1 ; c1ccc(-c2nc(-c3ccc4ccc5sc6ccccc6c5c4c3)nc(-c3cccc4ccccc34)n2)cc1]

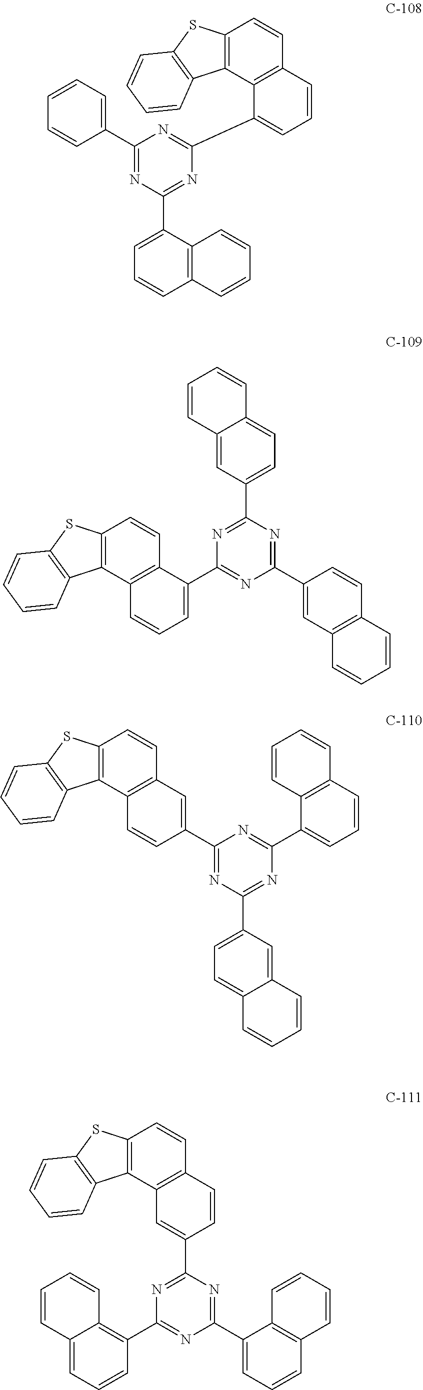 [SMILES: c1ccc(-c2nc(-c3cccc4ccccc34)nc(-c3cccc4ccc5sc6ccccc6c5c34)n2)cc1 ; c1ccc2cc(-c3nc(-c4ccc5ccccc5c4)nc(-c4cccc5c4ccc4sc6ccccc6c45)n3)ccc2c1 ; c1ccc2cc(-c3nc(-c4ccc5c(ccc6sc7ccccc7c65)c4)nc(-c4cccc5ccccc45)n3)ccc2c1 ; c1ccc2c(-c3nc(-c4ccc5ccc6sc7ccccc7c6c5c4)nc(-c4cccc5ccccc45)n3)cccc2c1]

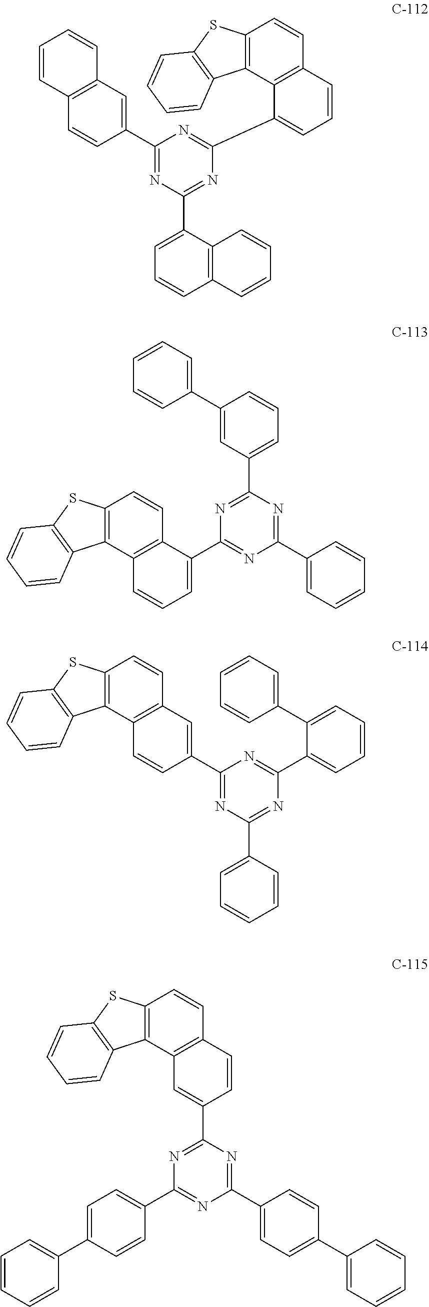 [SMILES: c1ccc2cc(-c3nc(-c4cccc5ccccc45)nc(-c4cccc5ccc6sc7ccccc7c6c45)n3)ccc2c1 ; c1ccc(-c2cccc(-c3nc(-c4ccccc4)nc(-c4cccc5c4ccc4sc6ccccc6c45)n3)c2)cc1 ; c1ccc(-c2nc(-c3ccc4c(ccc5sc6ccccc6c54)c3)nc(-c3ccccc3-c3ccccc3)n2)cc1 ; c1ccc(-c2ccc(-c3nc(-c4ccc(-c5ccccc5)cc4)nc(-c4ccc5ccc6sc7ccccc7c6c5c4)n3)cc2)cc1]

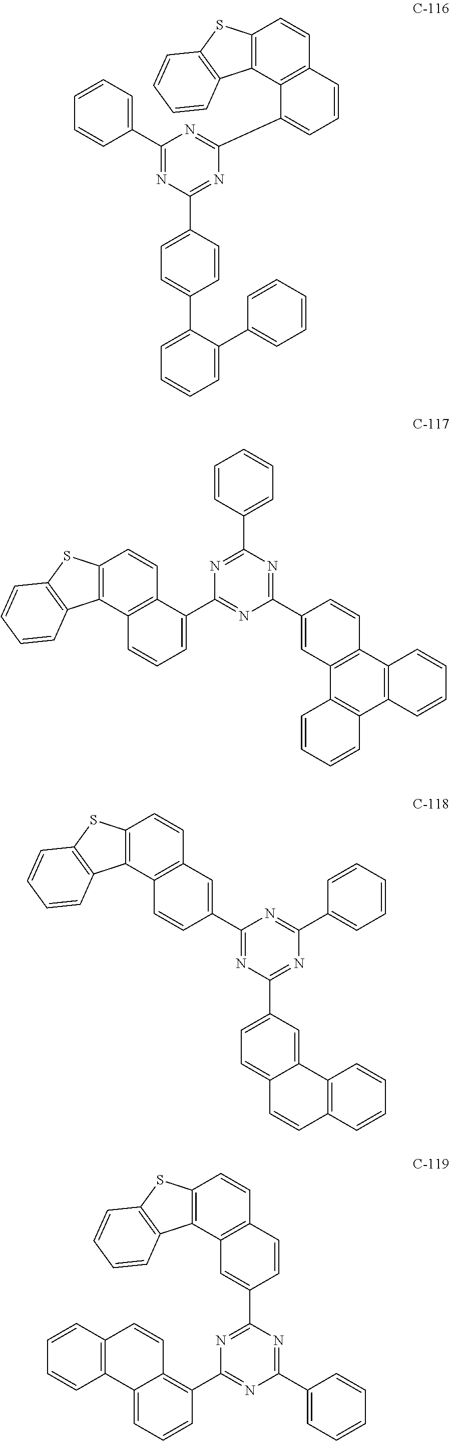 [SMILES: c1ccc(-c2nc(-c3ccc(-c4ccccc4-c4ccccc4)cc3)nc(-c3cccc4ccc5sc6ccccc6c5c34)n2)cc1 ; c1ccc(-c2nc(-c3ccc4c5ccccc5c5ccccc5c4c3)nc(-c3cccc4c3ccc3sc5ccccc5c34)n2)cc1 ; c1ccc(-c2nc(-c3ccc4c(ccc5sc6ccccc6c54)c3)nc(-c3ccc4ccc5ccccc5c4c3)n2)cc1 ; c1ccc(-c2nc(-c3ccc4ccc5sc6ccccc6c5c4c3)nc(-c3cccc4c3ccc3ccccc34)n2)cc1]

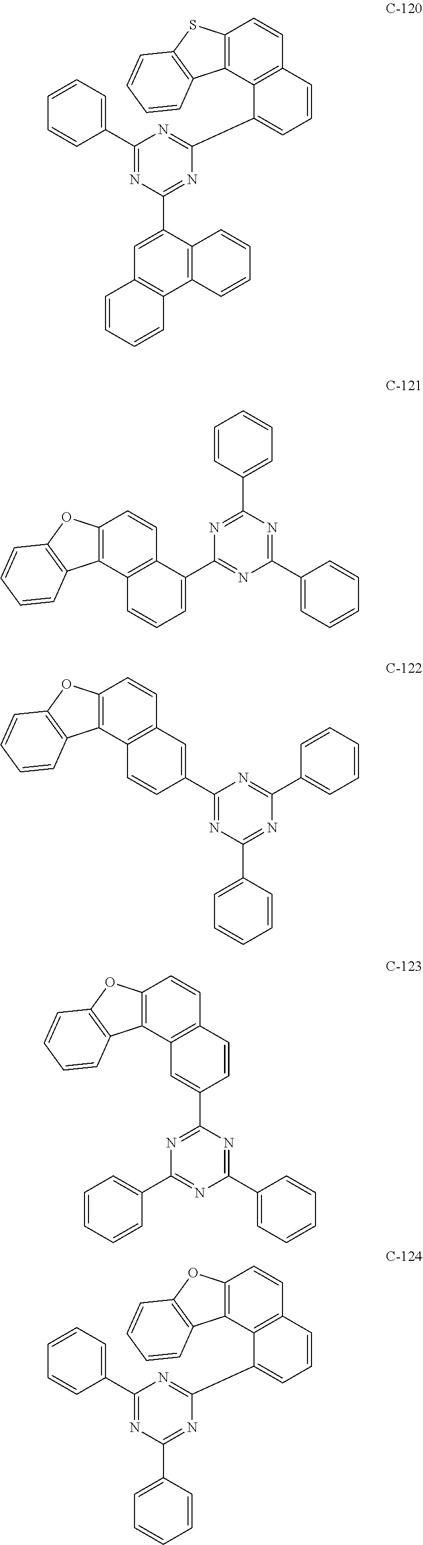

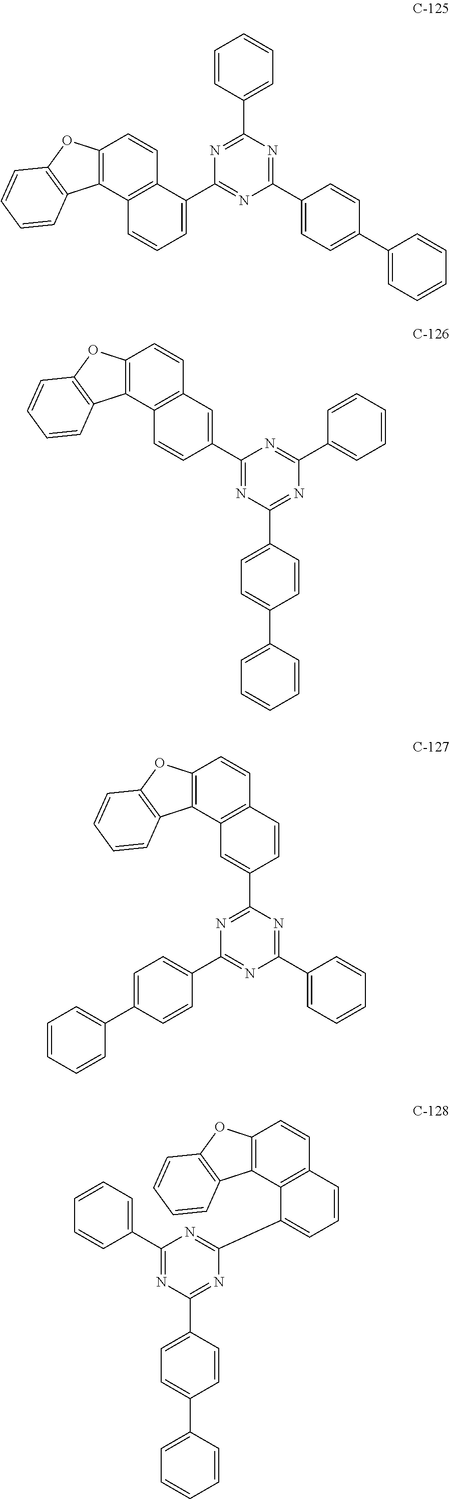 [SMILES: c1ccc(-c2ccc(-c3nc(-c4ccccc4)nc(-c4cccc5c4ccc4oc6ccccc6c45)n3)cc2)cc1 ; c1ccc(-c2ccc(-c3nc(-c4ccccc4)nc(-c4ccc5c(ccc6oc7ccccc7c65)c4)n3)cc2)cc1 ; c1ccc(-c2ccc(-c3nc(-c4ccccc4)nc(-c4ccc5ccc6oc7ccccc7c6c5c4)n3)cc2)cc1 ; c1ccc(-c2ccc(-c3nc(-c4ccccc4)nc(-c4cccc5ccc6oc7ccccc7c6c45)n3)cc2)cc1]

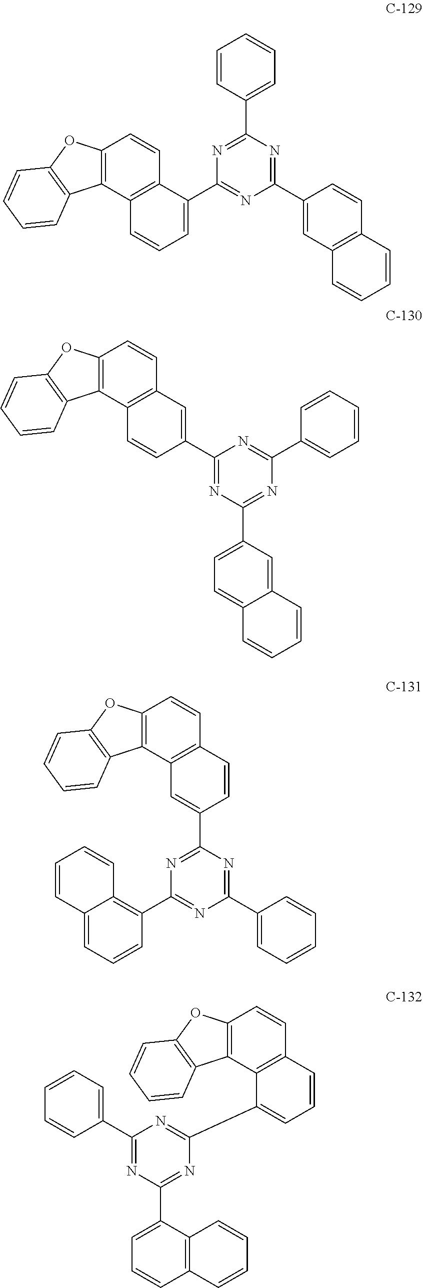 [SMILES: c1ccc(-c2nc(-c3ccc4ccccc4c3)nc(-c3cccc4c3ccc3oc5ccccc5c34)n2)cc1 ; c1ccc(-c2nc(-c3ccc4ccccc4c3)nc(-c3ccc4c(ccc5oc6ccccc6c54)c3)n2)cc1 ; c1ccc(-c2nc(-c3ccc4ccc5oc6ccccc6c5c4c3)nc(-c3cccc4ccccc34)n2)cc1 ; c1ccc(-c2nc(-c3cccc4ccccc34)nc(-c3cccc4ccc5oc6ccccc6c5c34)n2)cc1]

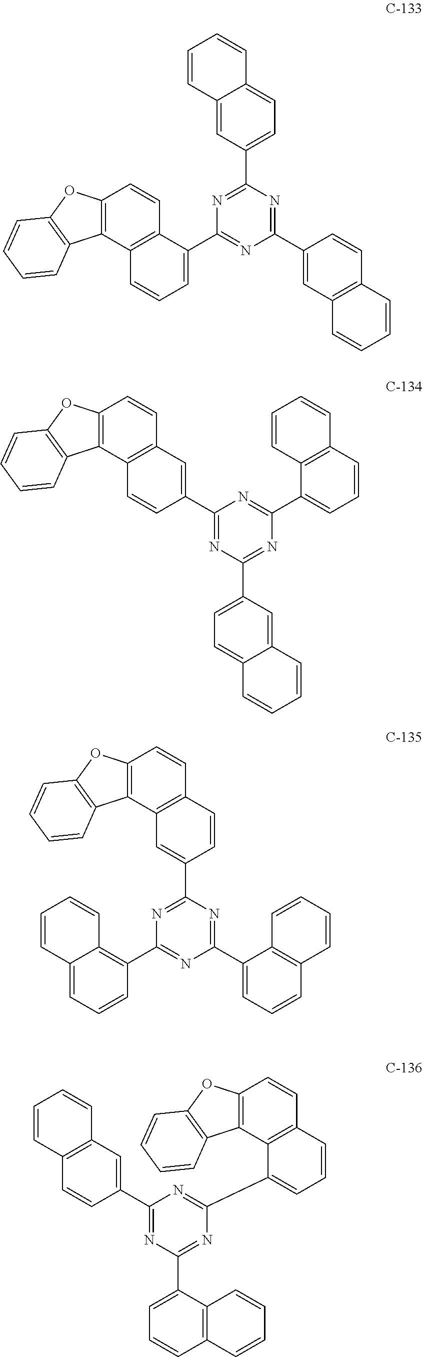 [SMILES: c1ccc2cc(-c3nc(-c4ccc5ccccc5c4)nc(-c4cccc5c4ccc4oc6ccccc6c45)n3)ccc2c1 ; c1ccc2cc(-c3nc(-c4ccc5c(ccc6oc7ccccc7c65)c4)nc(-c4cccc5ccccc45)n3)ccc2c1 ; c1ccc2c(-c3nc(-c4ccc5ccc6oc7ccccc7c6c5c4)nc(-c4cccc5ccccc45)n3)cccc2c1 ; c1ccc2cc(-c3nc(-c4cccc5ccccc45)nc(-c4cccc5ccc6oc7ccccc7c6c45)n3)ccc2c1]

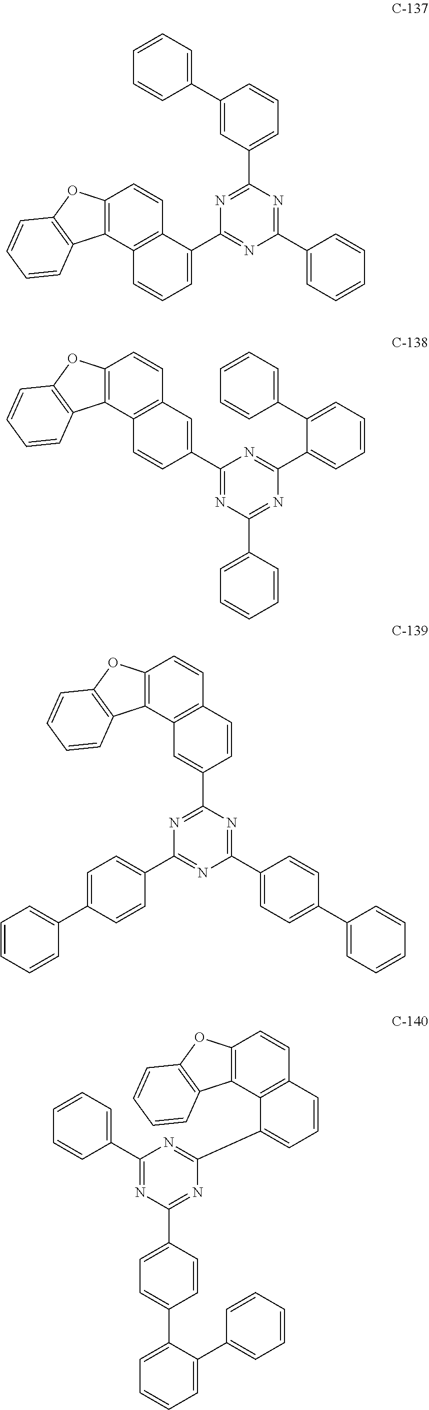 [SMILES: c1ccc(-c2cccc(-c3nc(-c4ccccc4)nc(-c4cccc5c4ccc4oc6ccccc6c45)n3)c2)cc1 ; c1ccc(-c2nc(-c3ccc4c(ccc5oc6ccccc6c54)c3)nc(-c3ccccc3-c3ccccc3)n2)cc1 ; c1ccc(-c2ccc(-c3nc(-c4ccc(-c5ccccc5)cc4)nc(-c4ccc5ccc6oc7ccccc7c6c5c4)n3)cc2)cc1 ; c1ccc(-c2nc(-c3ccc(-c4ccccc4-c4ccccc4)cc3)nc(-c3cccc4ccc5oc6ccccc6c5c34)n2)cc1]

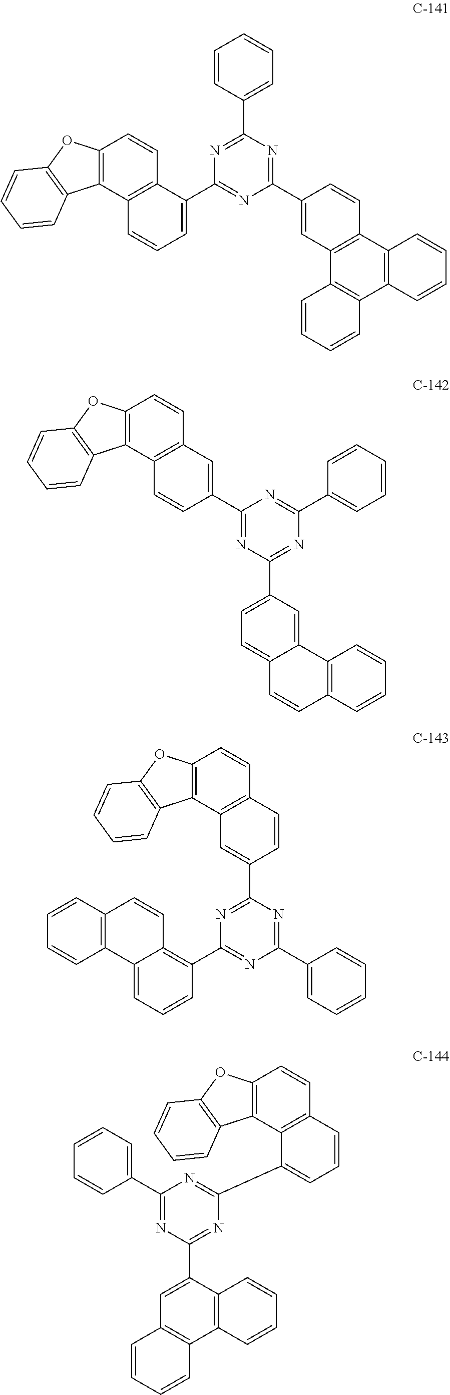 [SMILES: c1ccc(-c2nc(-c3ccc4c5ccccc5c5ccccc5c4c3)nc(-c3cccc4c3ccc3oc5ccccc5c34)n2)cc1 ; c1ccc(-c2nc(-c3ccc4c(ccc5oc6ccccc6c54)c3)nc(-c3ccc4ccc5ccccc5c4c3)n2)cc1 ; c1ccc(-c2nc(-c3ccc4ccc5oc6ccccc6c5c4c3)nc(-c3cccc4c3ccc3ccccc34)n2)cc1 ; c1ccc(-c2nc(-c3cc4ccccc4c4ccccc34)nc(-c3cccc4ccc5oc6ccccc6c5c34)n2)cc1]

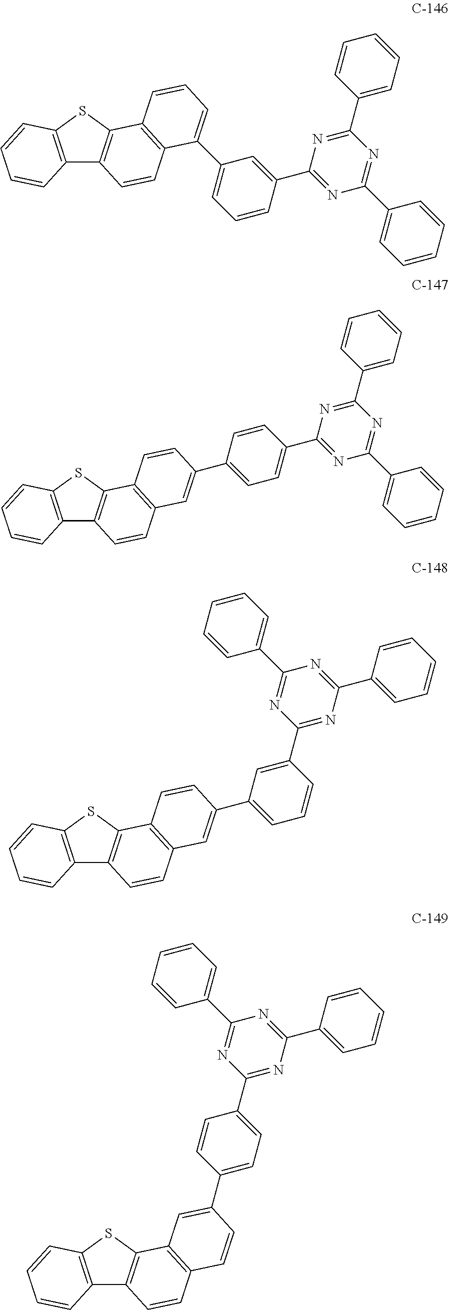 [SMILES: c1ccc(-c2nc(-c3ccccc3)nc(-c3cccc(-c4cccc5c4ccc4c6ccccc6sc54)c3)n2)cc1 ; c1ccc(-c2nc(-c3ccccc3)nc(-c3ccc(-c4ccc5c(ccc6c7ccccc7sc56)c4)cc3)n2)cc1 ; c1ccc(-c2nc(-c3ccccc3)nc(-c3cccc(-c4ccc5c(ccc6c7ccccc7sc56)c4)c3)n2)cc1 ; c1ccc(-c2nc(-c3ccccc3)nc(-c3ccc(-c4ccc5ccc6c7ccccc7sc6c5c4)cc3)n2)cc1]

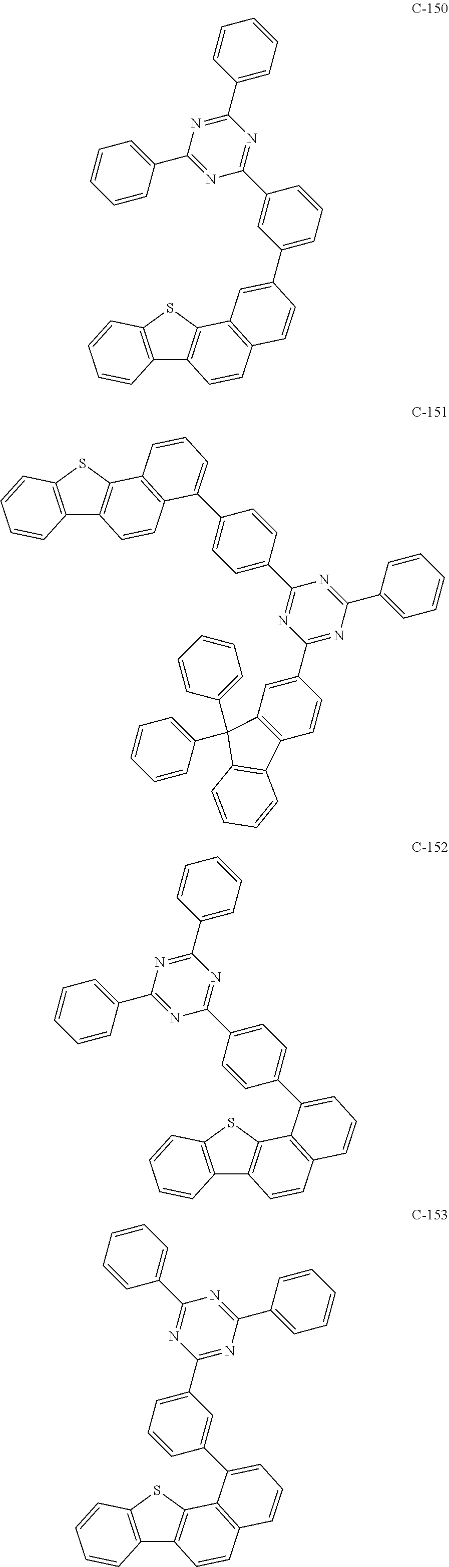

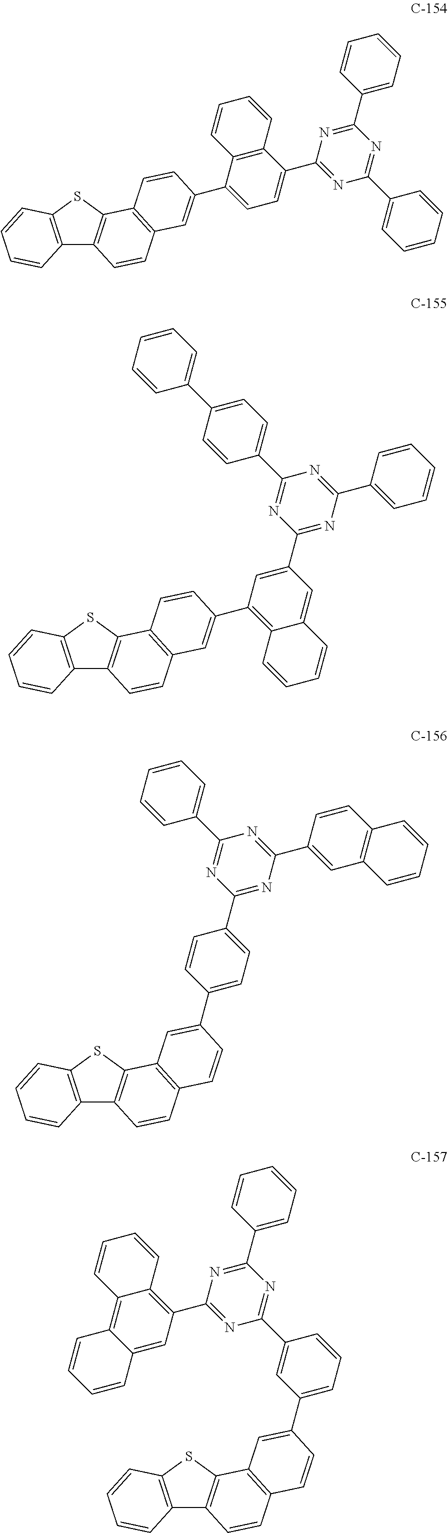 [SMILES: c1ccc(-c2nc(-c3ccccc3)nc(-c3ccc(-c4ccc5c(ccc6c7ccccc7sc56)c4)c4ccccc34)n2)cc1 ; c1ccc(-c2ccc(-c3nc(-c4ccccc4)nc(-c4cc(-c5ccc6c(ccc7c8ccccc8sc67)c5)c5ccccc5c4)n3)cc2)cc1 ; c1ccc(-c2nc(-c3ccc(-c4ccc5ccc6c7ccccc7sc6c5c4)cc3)nc(-c3ccc4ccccc4c3)n2)cc1 ; c1ccc(-c2nc(-c3cccc(-c4ccc5ccc6c7ccccc7sc6c5c4)c3)nc(-c3cc4ccccc4c4ccccc34)n2)cc1]

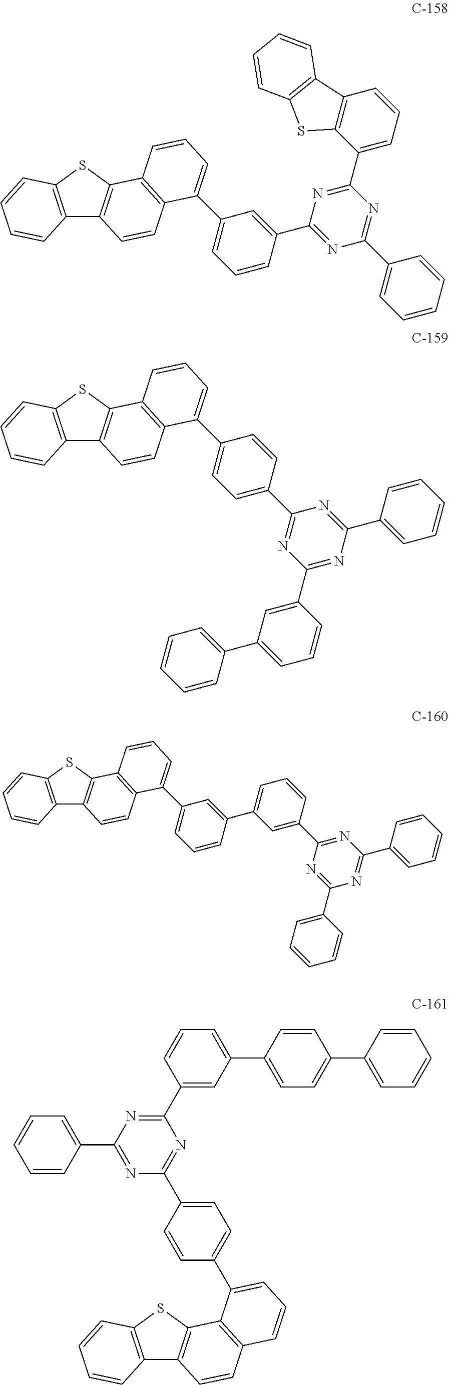 [SMILES: c1ccc(-c2nc(-c3cccc(-c4cccc5c4ccc4c6ccccc6sc54)c3)nc(-c3cccc4c3sc3ccccc34)n2)cc1 ; c1ccc(-c2cccc(-c3nc(-c4ccccc4)nc(-c4ccc(-c5cccc6c5ccc5c7ccccc7sc65)cc4)n3)c2)cc1 ; c1ccc(-c2nc(-c3ccccc3)nc(-c3cccc(-c4cccc(-c5cccc6c5ccc5c7ccccc7sc65)c4)c3)n2)cc1 ; c1ccc(-c2ccc(-c3cccc(-c4nc(-c5ccccc5)nc(-c5ccc(-c6cccc7ccc8c9ccccc9sc8c67)cc5)n4)c3)cc2)cc1]

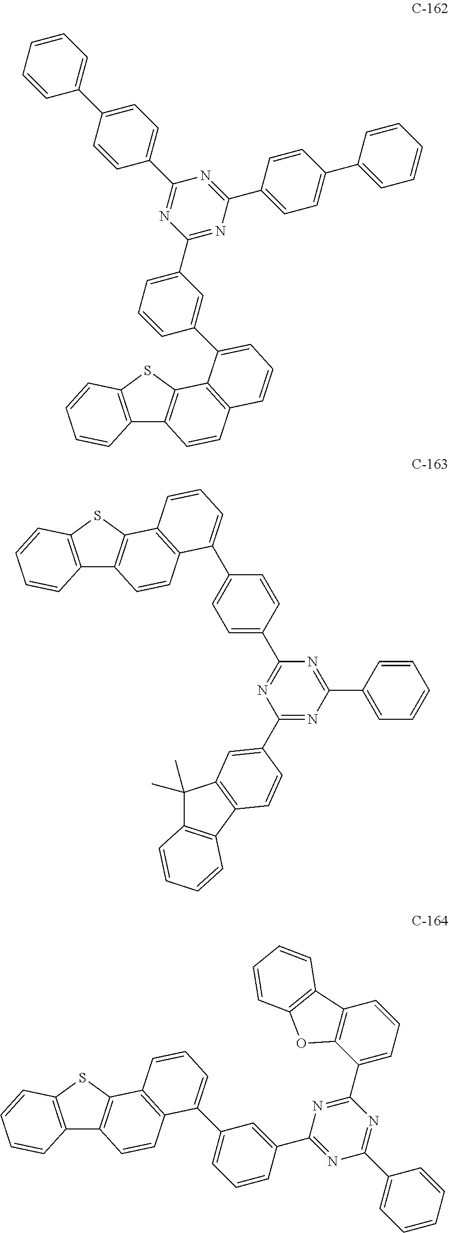

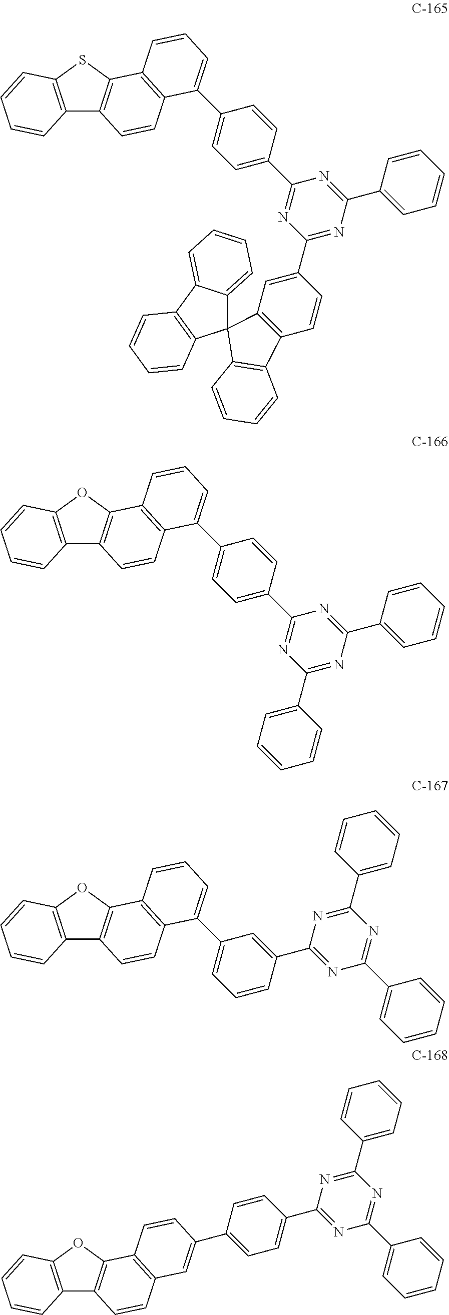

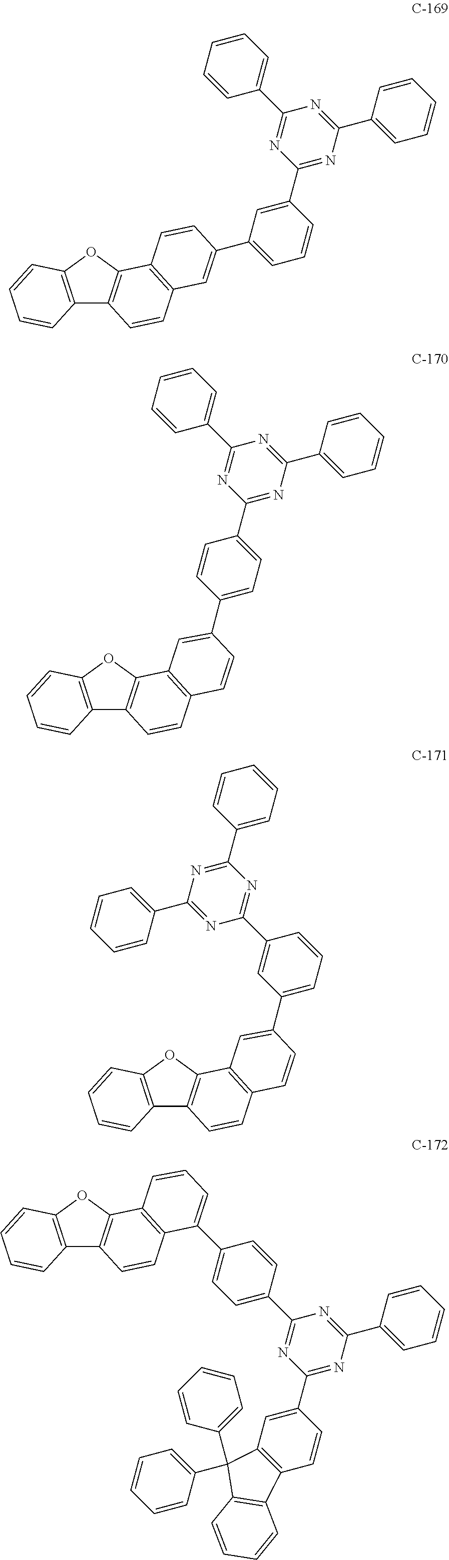

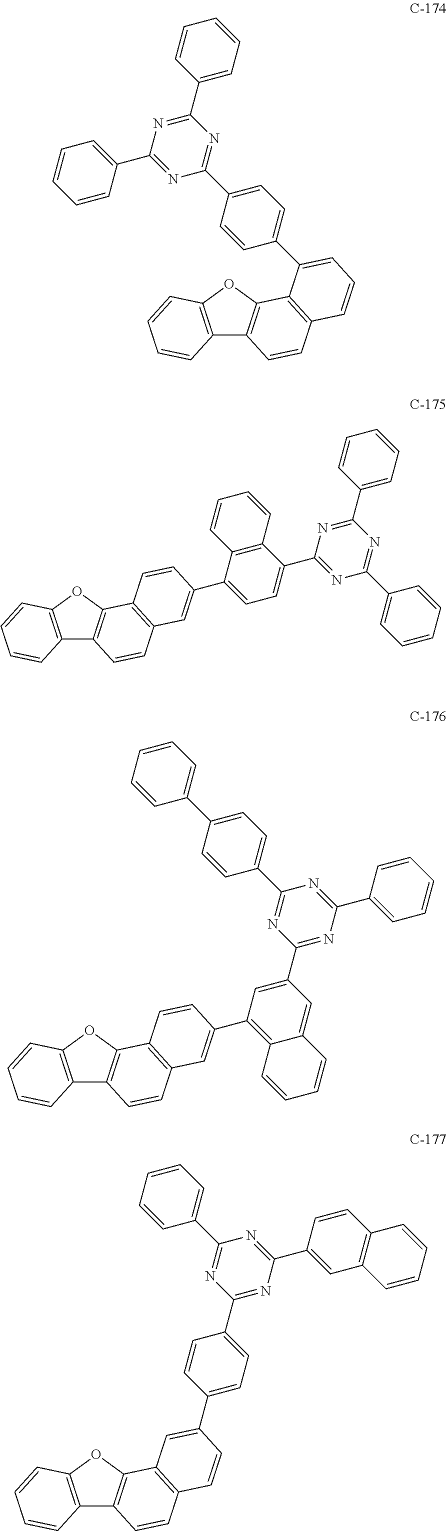 [SMILES: c1ccc(-c2nc(-c3ccccc3)nc(-c3ccc(-c4cccc5ccc6c7ccccc7oc6c45)cc3)n2)cc1 ; c1ccc(-c2nc(-c3ccccc3)nc(-c3ccc(-c4ccc5c(ccc6c7ccccc7oc56)c4)c4ccccc34)n2)cc1 ; c1ccc(-c2ccc(-c3nc(-c4ccccc4)nc(-c4cc(-c5ccc6c(ccc7c8ccccc8oc67)c5)c5ccccc5c4)n3)cc2)cc1 ; c1ccc(-c2nc(-c3ccc(-c4ccc5ccc6c7ccccc7oc6c5c4)cc3)nc(-c3ccc4ccccc4c3)n2)cc1]

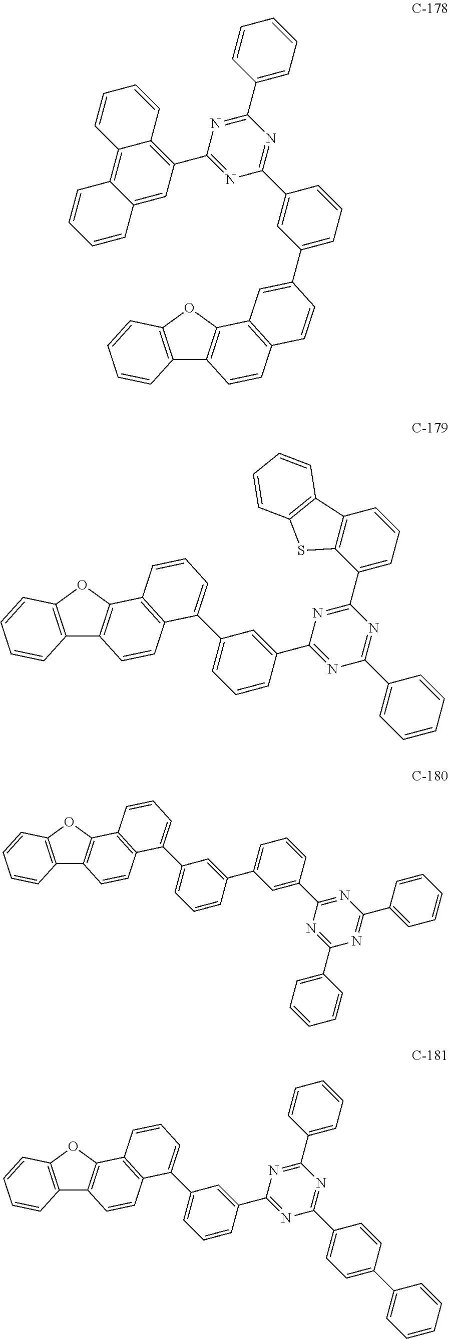 [SMILES: c1ccc(-c2nc(-c3cccc(-c4ccc5ccc6c7ccccc7oc6c5c4)c3)nc(-c3cc4ccccc4c4ccccc34)n2)cc1 ; c1ccc(-c2nc(-c3cccc(-c4cccc5c4ccc4c6ccccc6oc54)c3)nc(-c3cccc4c3sc3ccccc34)n2)cc1 ; c1ccc(-c2nc(-c3ccccc3)nc(-c3cccc(-c4cccc(-c5cccc6c5ccc5c7ccccc7oc65)c4)c3)n2)cc1 ; c1ccc(-c2ccc(-c3nc(-c4ccccc4)nc(-c4cccc(-c5cccc6c5ccc5c7ccccc7oc65)c4)n3)cc2)cc1]

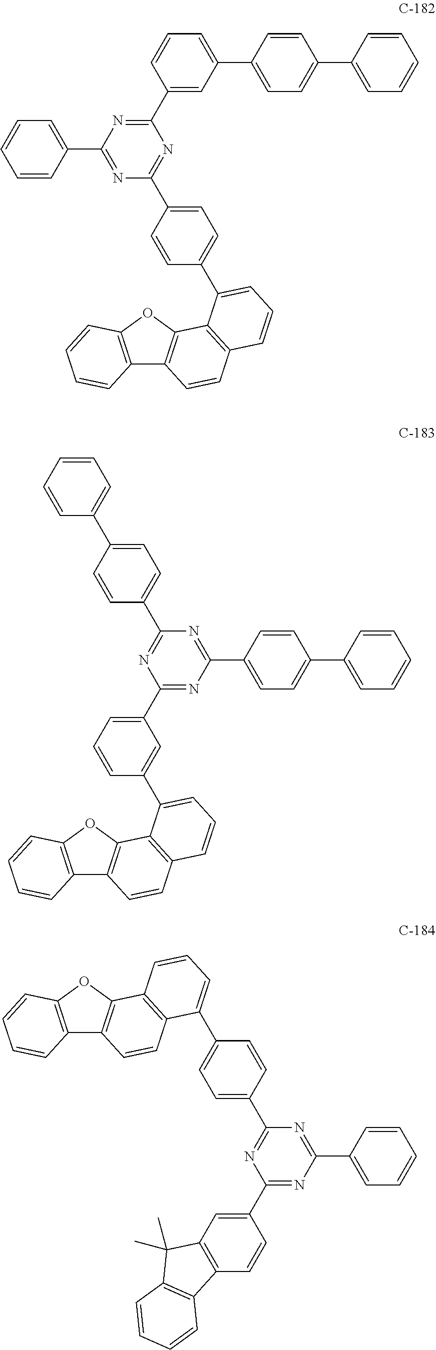 [SMILES: c1ccc(-c2ccc(-c3cccc(-c4nc(-c5ccccc5)nc(-c5ccc(-c6cccc7ccc8c9ccccc9oc8c67)cc5)n4)c3)cc2)cc1 ; c1ccc(-c2ccc(-c3nc(-c4ccc(-c5ccccc5)cc4)nc(-c4cccc(-c5cccc6ccc7c8ccccc8oc7c56)c4)n3)cc2)cc1 ; CC1(C)c2ccccc2-c2ccc(-c3nc(-c4ccccc4)nc(-c4ccc(-c5cccc6c5ccc5c7ccccc7oc65)cc4)n3)cc21]

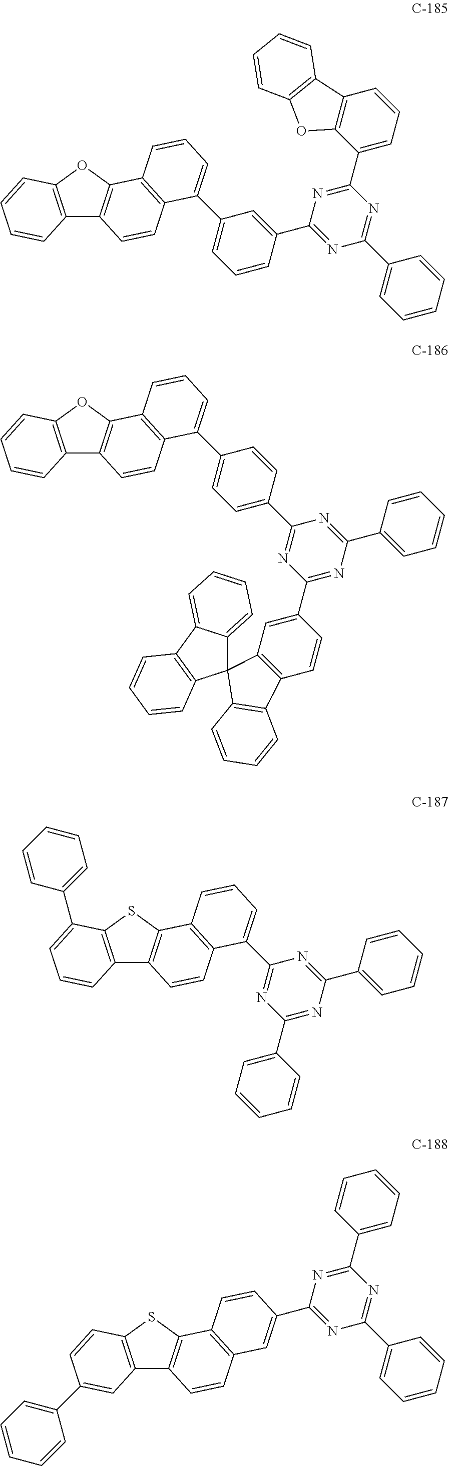 [SMILES: c1ccc(-c2nc(-c3cccc(-c4cccc5c4ccc4c6ccccc6oc54)c3)nc(-c3cccc4c3oc3ccccc34)n2)cc1 ; c1ccc(-c2nc(-c3ccc(-c4cccc5c4ccc4c6ccccc6oc54)cc3)nc(-c3ccc4c(c3)C3(c5ccccc5-c5ccccc53)c3ccccc3-4)n2)cc1 ; c1ccc(-c2nc(-c3ccccc3)nc(-c3cccc4c3ccc3c5cccc(-c6ccccc6)c5sc43)n2)cc1 ; c1ccc(-c2ccc3sc4c5ccc(-c6nc(-c7ccccc7)nc(-c7ccccc7)n6)cc5ccc4c3c2)cc1]

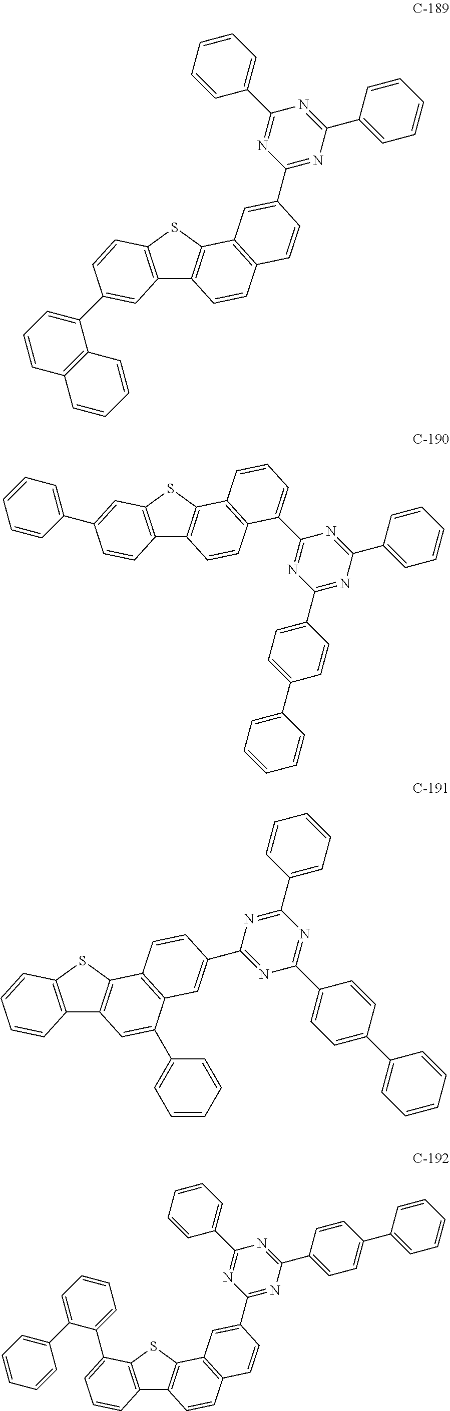 [SMILES: c1ccc(-c2nc(-c3ccccc3)nc(-c3ccc4ccc5c6cc(-c7cccc8ccccc78)ccc6sc5c4c3)n2)cc1 ; c1ccc(-c2ccc(-c3nc(-c4ccccc4)nc(-c4cccc5c4ccc4c6ccc(-c7ccccc7)cc6sc54)n3)cc2)cc1 ; c1ccc(-c2ccc(-c3nc(-c4ccccc4)nc(-c4ccc5c(c4)c(-c4ccccc4)cc4c6ccccc6sc54)n3)cc2)cc1 ; c1ccc(-c2ccc(-c3nc(-c4ccccc4)nc(-c4ccc5ccc6c7cccc(-c8ccccc8-c8ccccc8)c7sc6c5c4)n3)cc2)cc1]

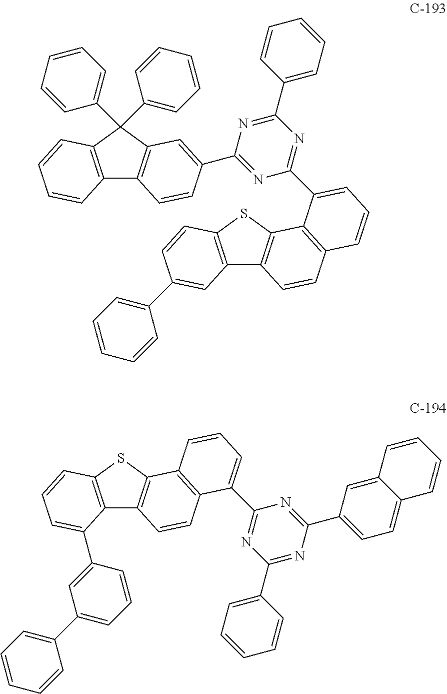 [SMILES: c1ccc(-c2ccc3sc4c(ccc5cccc(-c6nc(-c7ccccc7)nc(-c7ccc8c(c7)C(c7ccccc7)(c7ccccc7)c7ccccc7-8)n6)c54)c3c2)cc1 ; c1ccc(-c2cccc(-c3cccc4sc5c6cccc(-c7nc(-c8ccccc8)nc(-c8ccc9ccccc9c8)n7)c6ccc5c34)c2)cc1]

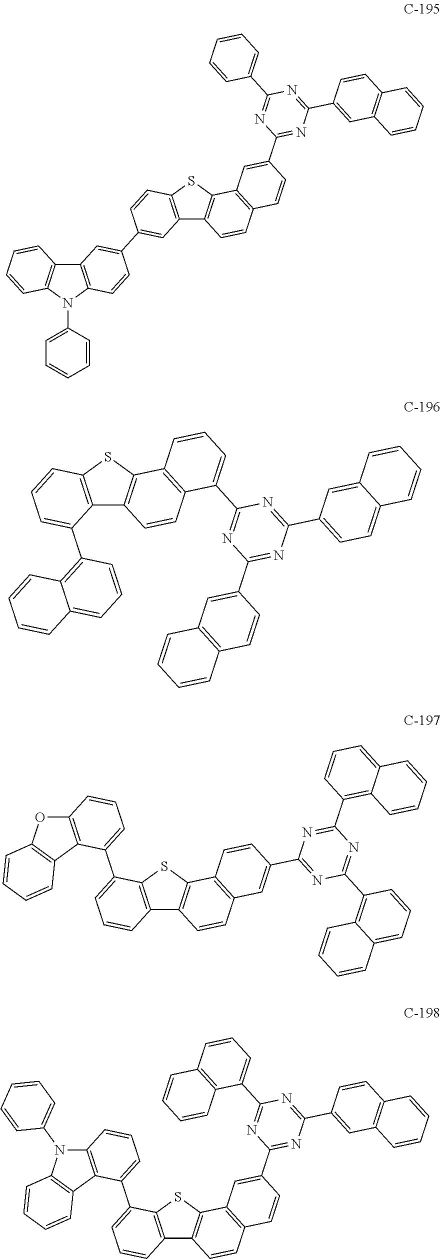 [SMILES: c1ccc(-c2nc(-c3ccc4ccccc4c3)nc(-c3ccc4ccc5c6cc(-c7ccc8c(c7)c7ccccc7n8-c7ccccc7)ccc6sc5c4c3)n2)cc1 ; c1ccc2cc(-c3nc(-c4ccc5ccccc5c4)nc(-c4cccc5c4ccc4c5sc5cccc(-c6cccc7ccccc67)c54)n3)ccc2c1 ; c1ccc2c(-c3nc(-c4ccc5c(ccc6c7cccc(-c8cccc9oc%10ccccc%10c89)c7sc56)c4)nc(-c4cccc5ccccc45)n3)cccc2c1 ; c1ccc(-n2c3ccccc3c3c(-c4cccc5c4sc4c6cc(-c7nc(-c8ccc9ccccc9c8)nc(-c8cccc9ccccc89)n7)ccc6ccc54)cccc32)cc1]

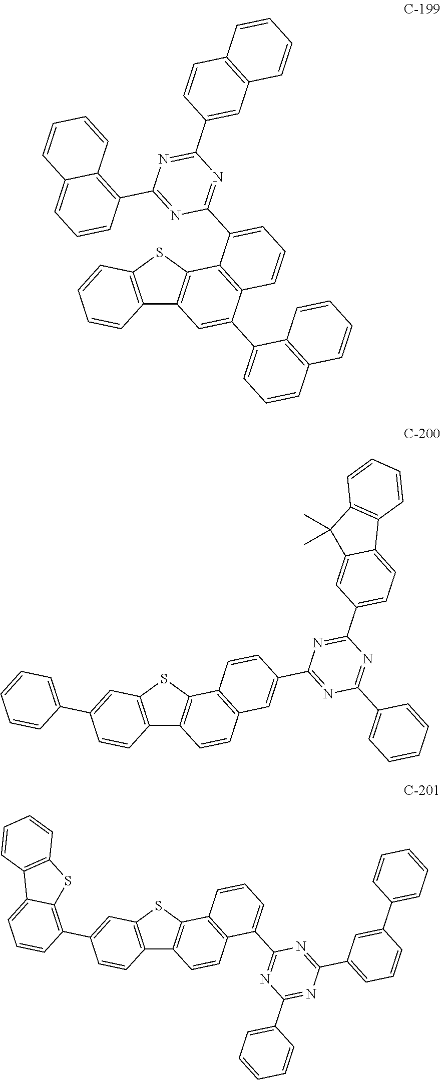 [SMILES: c1ccc2cc(-c3nc(-c4cccc5ccccc45)nc(-c4cccc5c(-c6cccc7ccccc67)cc6c7ccccc7sc6c45)n3)ccc2c1 ; CC1(C)c2ccccc2-c2ccc(-c3nc(-c4ccccc4)nc(-c4ccc5c(ccc6c7ccc(-c8ccccc8)cc7sc56)c4)n3)cc21 ; c1ccc(-c2cccc(-c3nc(-c4ccccc4)nc(-c4cccc5c4ccc4c6ccc(-c7cccc8c7sc7ccccc78)cc6sc54)n3)c2)cc1]

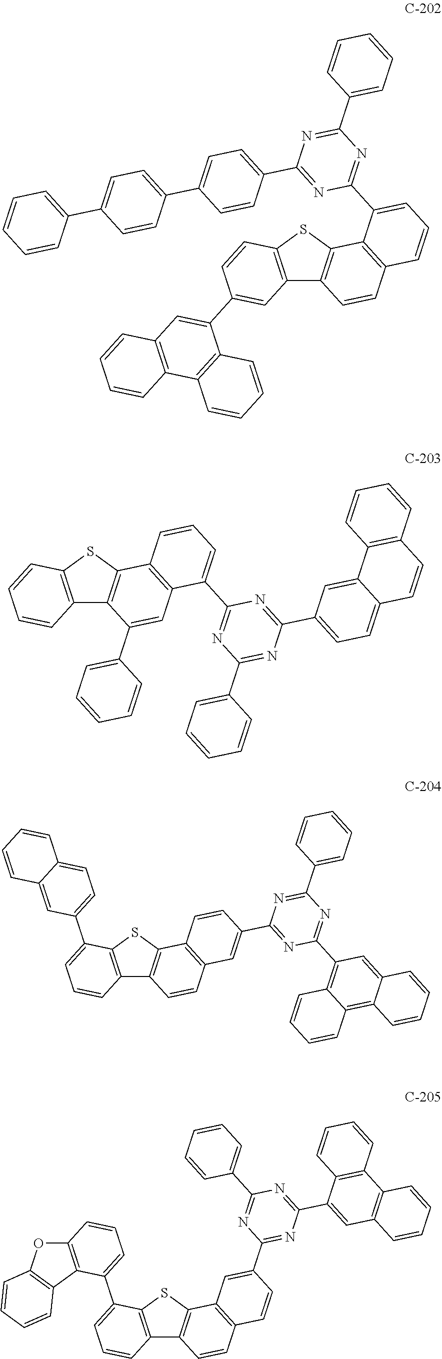 [SMILES: c1ccc(-c2ccc(-c3ccc(-c4nc(-c5ccccc5)nc(-c5cccc6ccc7c8cc(-c9cc%10ccccc%10c%10ccccc9%10)ccc8sc7c56)n4)cc3)cc2)cc1 ; c1ccc(-c2nc(-c3ccc4ccc5ccccc5c4c3)nc(-c3cccc4c3cc(-c3ccccc3)c3c5ccccc5sc43)n2)cc1 ; c1ccc(-c2nc(-c3ccc4c(ccc5c6cccc(-c7ccc8ccccc8c7)c6sc45)c3)nc(-c3cc4ccccc4c4ccccc34)n2)cc1 ; c1ccc(-c2nc(-c3ccc4ccc5c6cccc(-c7cccc8oc9ccccc9c78)c6sc5c4c3)nc(-c3cc4ccccc4c4ccccc34)n2)cc1]

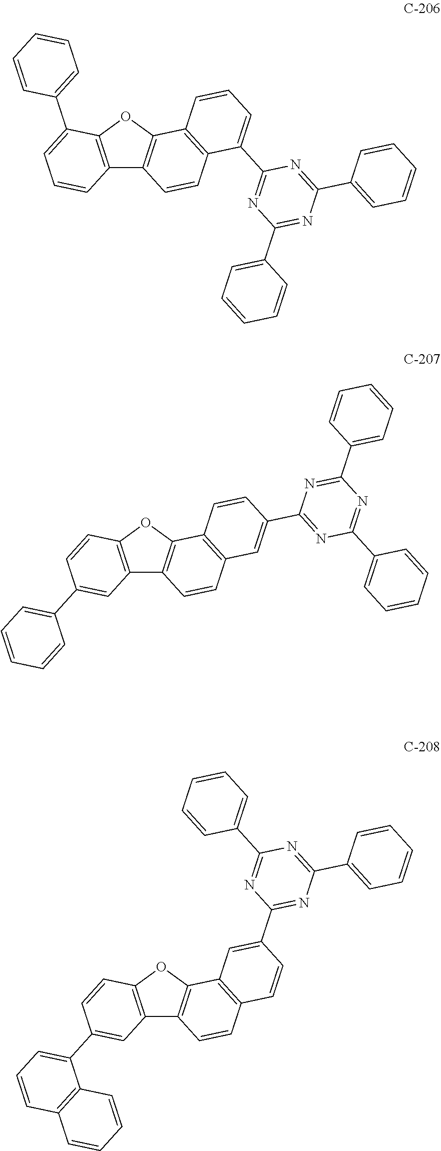 [SMILES: c1ccc(-c2nc(-c3ccccc3)nc(-c3cccc4c3ccc3c5cccc(-c6ccccc6)c5oc43)n2)cc1 ; c1ccc(-c2ccc3oc4c5ccc(-c6nc(-c7ccccc7)nc(-c7ccccc7)n6)cc5ccc4c3c2)cc1 ; c1ccc(-c2nc(-c3ccccc3)nc(-c3ccc4ccc5c6cc(-c7cccc8ccccc78)ccc6oc5c4c3)n2)cc1]

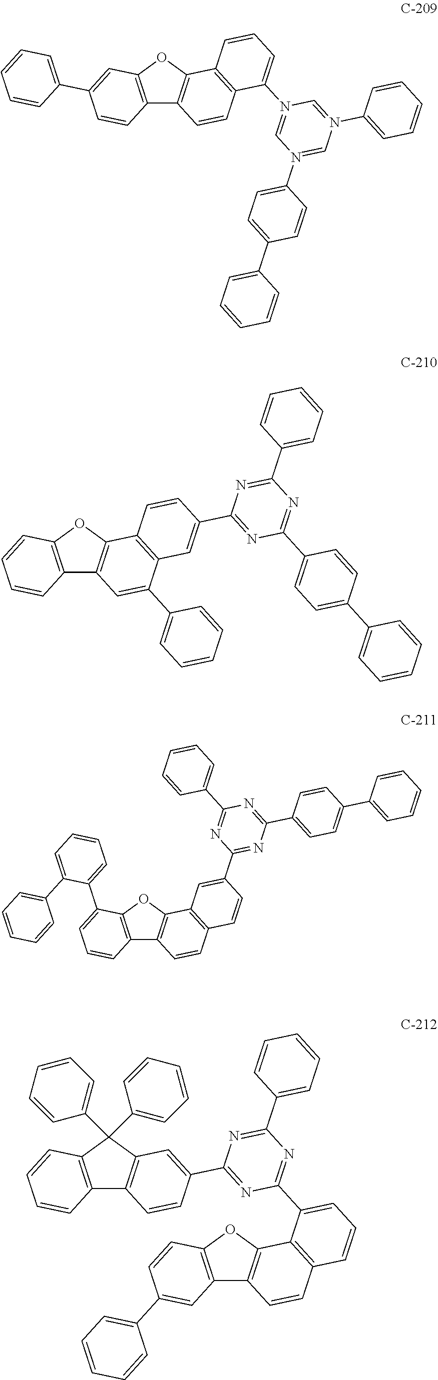

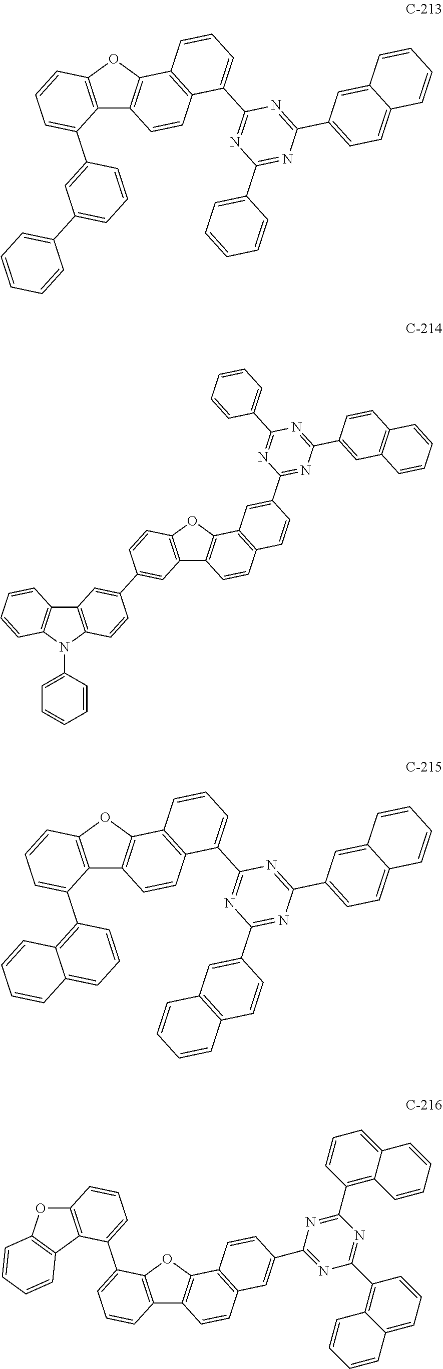 [SMILES: c1ccc(-c2cccc(-c3cccc4oc5c6cccc(-c7nc(-c8ccccc8)nc(-c8ccc9ccccc9c8)n7)c6ccc5c34)c2)cc1 ; c1ccc(-c2nc(-c3ccc4ccccc4c3)nc(-c3ccc4ccc5c6cc(-c7ccc8c(c7)c7ccccc7n8-c7ccccc7)ccc6oc5c4c3)n2)cc1 ; c1ccc2cc(-c3nc(-c4ccc5ccccc5c4)nc(-c4cccc5c4ccc4c5oc5cccc(-c6cccc7ccccc67)c54)n3)ccc2c1 ; c1ccc2c(-c3nc(-c4ccc5c(ccc6c7cccc(-c8cccc9oc%10ccccc%10c89)c7oc56)c4)nc(-c4cccc5ccccc45)n3)cccc2c1]

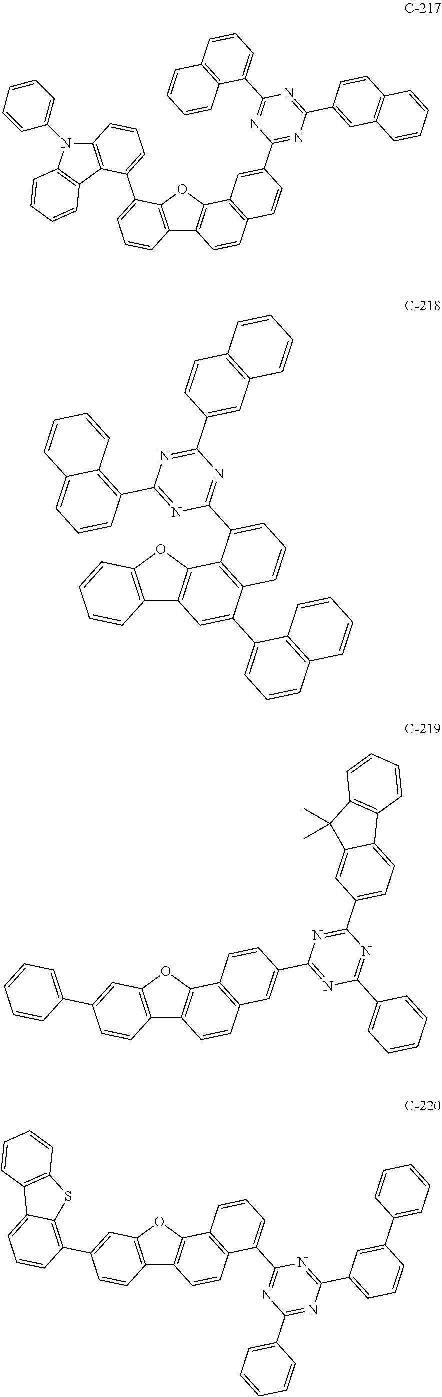 [SMILES: c1ccc(-n2c3ccccc3c3c(-c4cccc5c4oc4c6cc(-c7nc(-c8ccc9ccccc9c8)nc(-c8cccc9ccccc89)n7)ccc6ccc54)cccc32)cc1 ; c1ccc2cc(-c3nc(-c4cccc5ccccc45)nc(-c4cccc5c(-c6cccc7ccccc67)cc6c7ccccc7oc6c45)n3)ccc2c1 ; CC1(C)c2ccccc2-c2ccc(-c3nc(-c4ccccc4)nc(-c4ccc5c(ccc6c7ccc(-c8ccccc8)cc7oc56)c4)n3)cc21 ; c1ccc(-c2cccc(-c3nc(-c4ccccc4)nc(-c4cccc5c4ccc4c6ccc(-c7cccc8c7sc7ccccc78)cc6oc54)n3)c2)cc1]

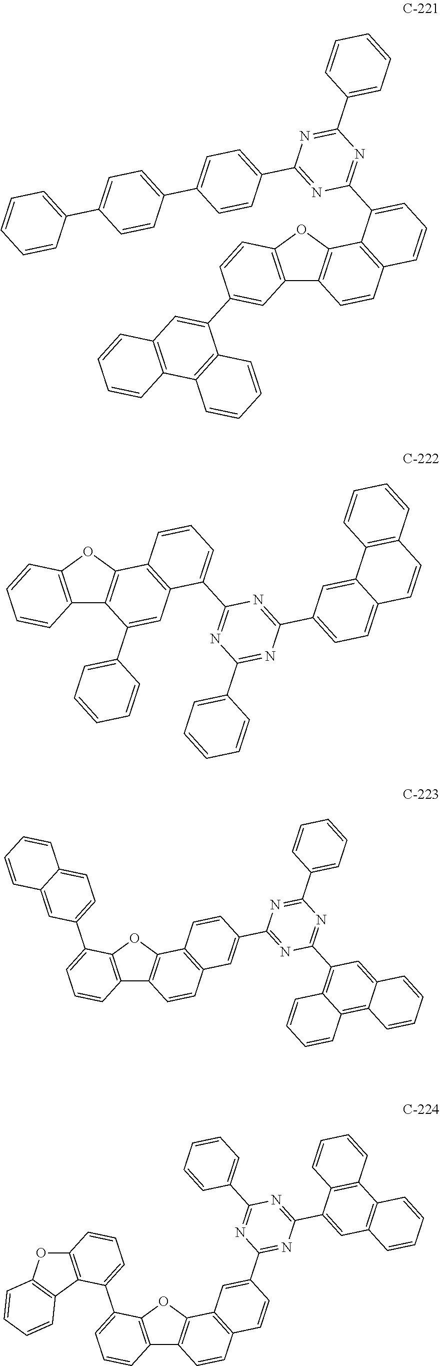 [SMILES: c1ccc(-c2ccc(-c3ccc(-c4nc(-c5ccccc5)nc(-c5cccc6ccc7c8cc(-c9cc%10ccccc%10c%10ccccc9%10)ccc8oc7c56)n4)cc3)cc2)cc1 ; c1ccc(-c2nc(-c3ccc4ccc5ccccc5c4c3)nc(-c3cccc4c3cc(-c3ccccc3)c3c5ccccc5oc43)n2)cc1 ; c1ccc(-c2nc(-c3ccc4c(ccc5c6cccc(-c7ccc8ccccc8c7)c6oc45)c3)nc(-c3cc4ccccc4c4ccccc34)n2)cc1 ; c1ccc(-c2nc(-c3ccc4ccc5c6cccc(-c7cccc8oc9ccccc9c78)c6oc5c4c3)nc(-c3cc4ccccc4c4ccccc34)n2)cc1]

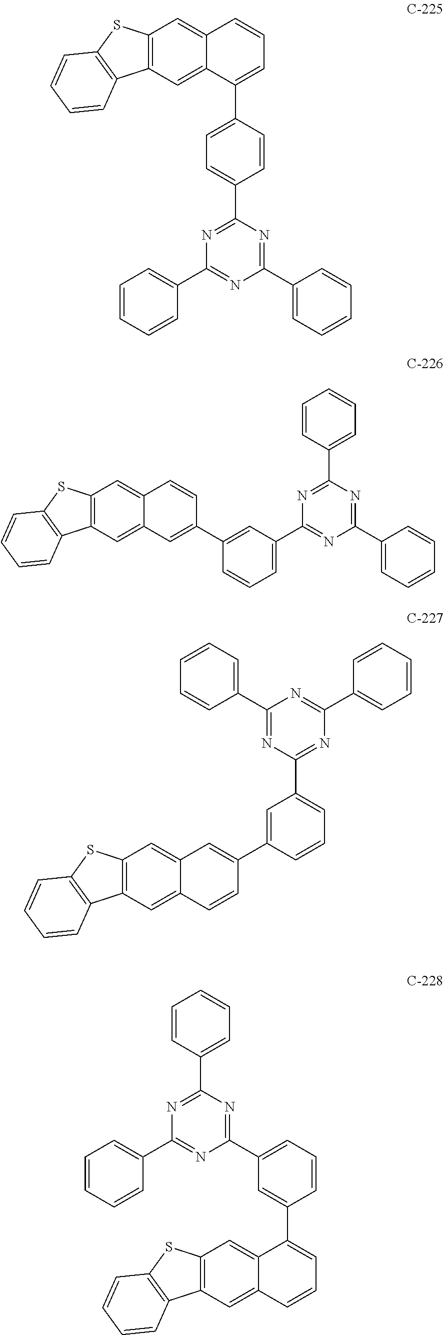 [SMILES: c1ccc(-c2nc(-c3ccccc3)nc(-c3ccc(-c4cccc5cc6sc7ccccc7c6cc45)cc3)n2)cc1 ; c1ccc(-c2nc(-c3ccccc3)nc(-c3cccc(-c4ccc5cc6sc7ccccc7c6cc5c4)c3)n2)cc1 ; c1ccc(-c2nc(-c3ccccc3)nc(-c3cccc(-c4ccc5cc6c(cc5c4)sc4ccccc46)c3)n2)cc1 ; c1ccc(-c2nc(-c3ccccc3)nc(-c3cccc(-c4cccc5cc6c(cc45)sc4ccccc46)c3)n2)cc1]

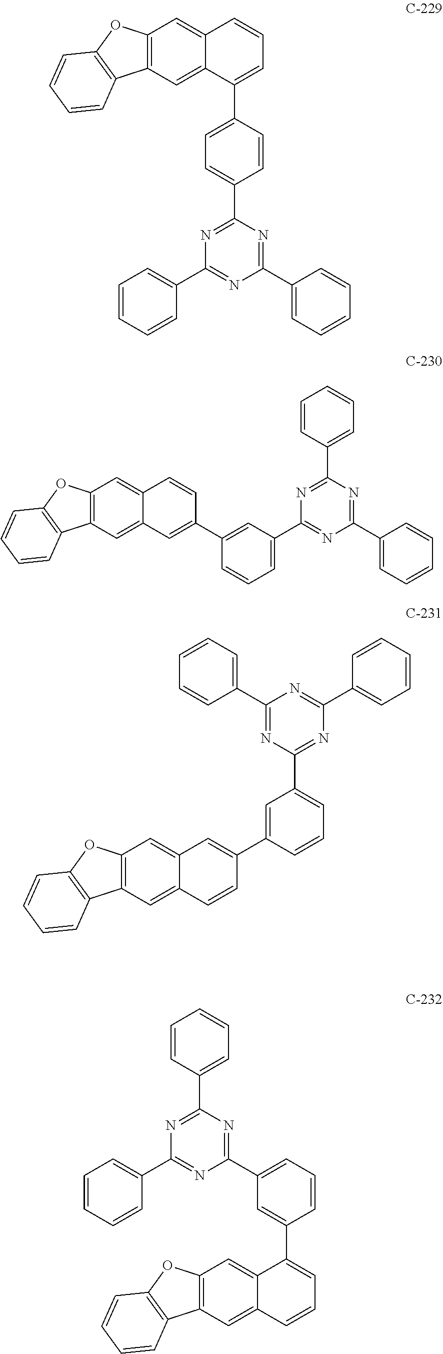 [SMILES: c1ccc(-c2nc(-c3ccccc3)nc(-c3ccc(-c4cccc5cc6oc7ccccc7c6cc45)cc3)n2)cc1 ; c1ccc(-c2nc(-c3ccccc3)nc(-c3cccc(-c4ccc5cc6oc7ccccc7c6cc5c4)c3)n2)cc1 ; c1ccc(-c2nc(-c3ccccc3)nc(-c3cccc(-c4ccc5cc6c(cc5c4)oc4ccccc46)c3)n2)cc1 ; c1ccc(-c2nc(-c3ccccc3)nc(-c3cccc(-c4cccc5cc6c(cc45)oc4ccccc46)c3)n2)cc1]

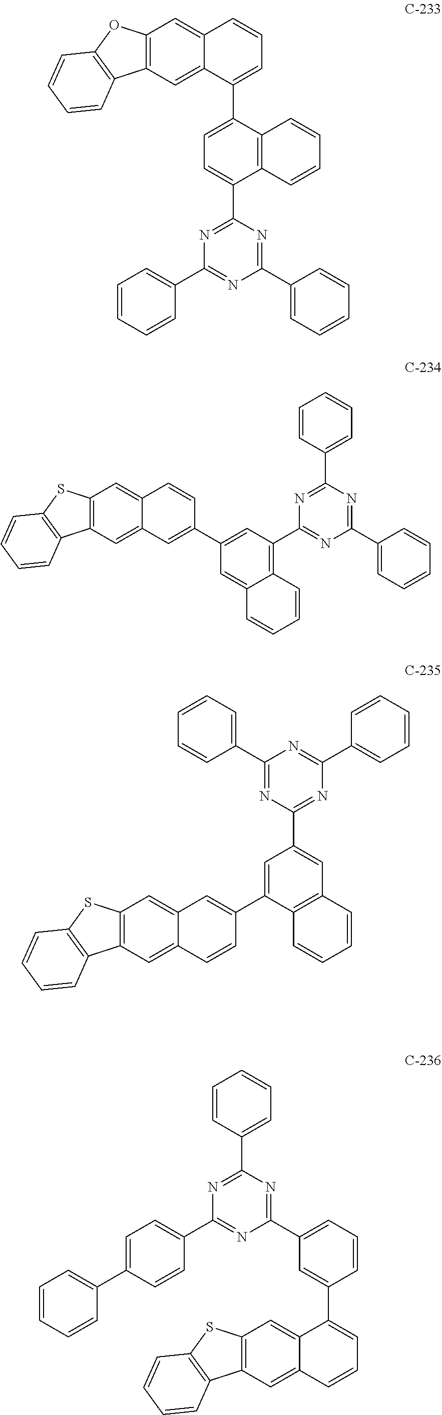 [SMILES: c1ccc(-c2nc(-c3ccccc3)nc(-c3ccc(-c4cccc5cc6oc7ccccc7c6cc45)c4ccccc34)n2)cc1 ; c1ccc(-c2nc(-c3ccccc3)nc(-c3cc(-c4ccc5cc6sc7ccccc7c6cc5c4)cc4ccccc34)n2)cc1 ; c1ccc(-c2nc(-c3ccccc3)nc(-c3cc(-c4ccc5cc6c(cc5c4)sc4ccccc46)c4ccccc4c3)n2)cc1 ; c1ccc(-c2ccc(-c3nc(-c4ccccc4)nc(-c4cccc(-c5cccc6cc7c(cc56)sc5ccccc57)c4)n3)cc2)cc1]

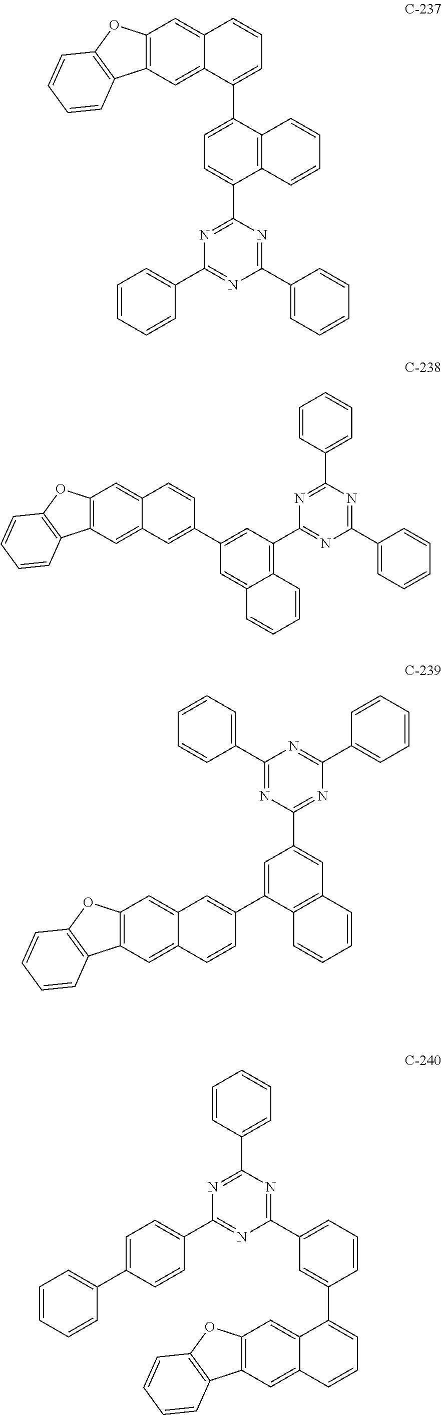 [SMILES: c1ccc(-c2nc(-c3ccccc3)nc(-c3ccc(-c4cccc5cc6oc7ccccc7c6cc45)c4ccccc34)n2)cc1 ; c1ccc(-c2nc(-c3ccccc3)nc(-c3cc(-c4ccc5cc6oc7ccccc7c6cc5c4)cc4ccccc34)n2)cc1 ; c1ccc(-c2nc(-c3ccccc3)nc(-c3cc(-c4ccc5cc6c(cc5c4)oc4ccccc46)c4ccccc4c3)n2)cc1 ; c1ccc(-c2ccc(-c3nc(-c4ccccc4)nc(-c4cccc(-c5cccc6cc7c(cc56)oc5ccccc57)c4)n3)cc2)cc1]

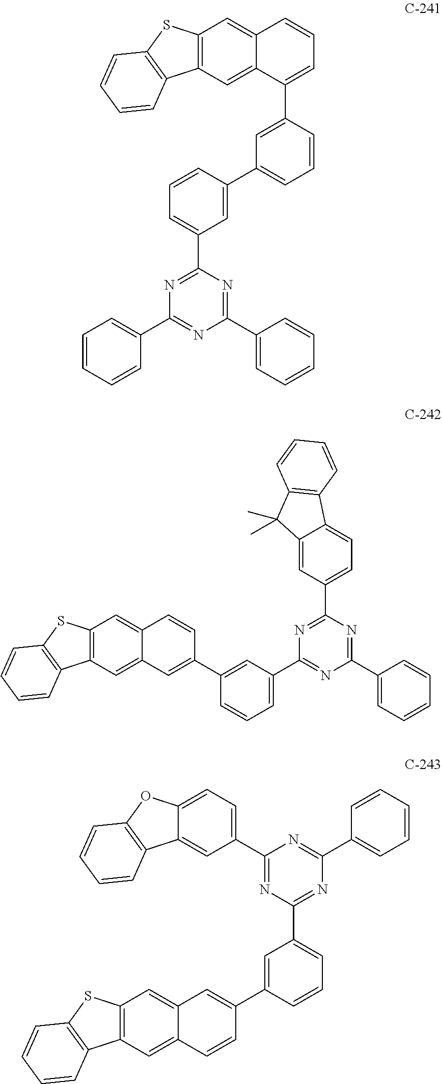 [SMILES: c1ccc(-c2nc(-c3ccccc3)nc(-c3cccc(-c4cccc(-c5cccc6cc7sc8ccccc8c7cc56)c4)c3)n2)cc1 ; CC1(C)c2ccccc2-c2ccc(-c3nc(-c4ccccc4)nc(-c4cccc(-c5ccc6cc7sc8ccccc8c7cc6c5)c4)n3)cc21 ; c1ccc(-c2nc(-c3cccc(-c4ccc5cc6c(cc5c4)sc4ccccc46)c3)nc(-c3ccc4oc5ccccc5c4c3)n2)cc1]

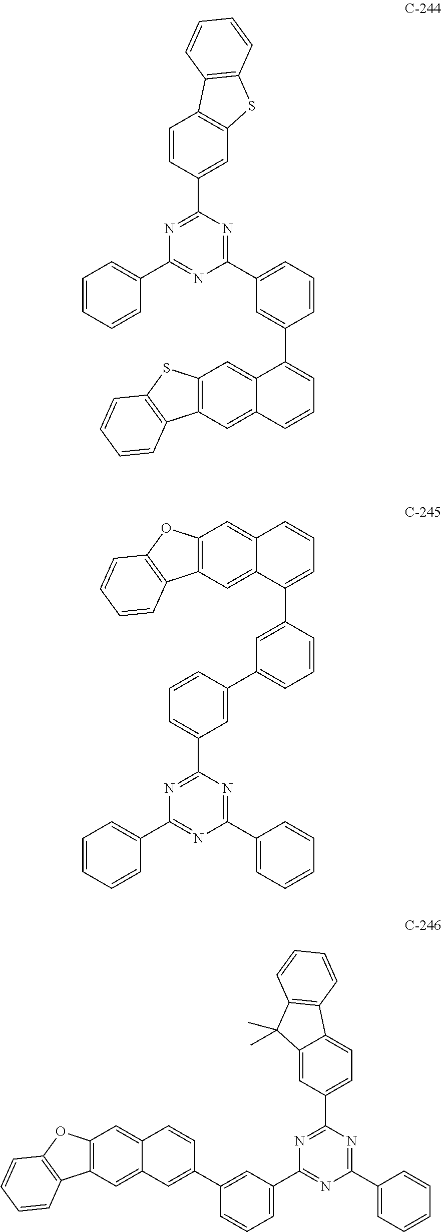 [SMILES: c1ccc(-c2nc(-c3cccc(-c4cccc5cc6c(cc45)sc4ccccc46)c3)nc(-c3ccc4c(c3)sc3ccccc34)n2)cc1 ; c1ccc(-c2nc(-c3ccccc3)nc(-c3cccc(-c4cccc(-c5cccc6cc7oc8ccccc8c7cc56)c4)c3)n2)cc1 ; CC1(C)c2ccccc2-c2ccc(-c3nc(-c4ccccc4)nc(-c4cccc(-c5ccc6cc7oc8ccccc8c7cc6c5)c4)n3)cc21]

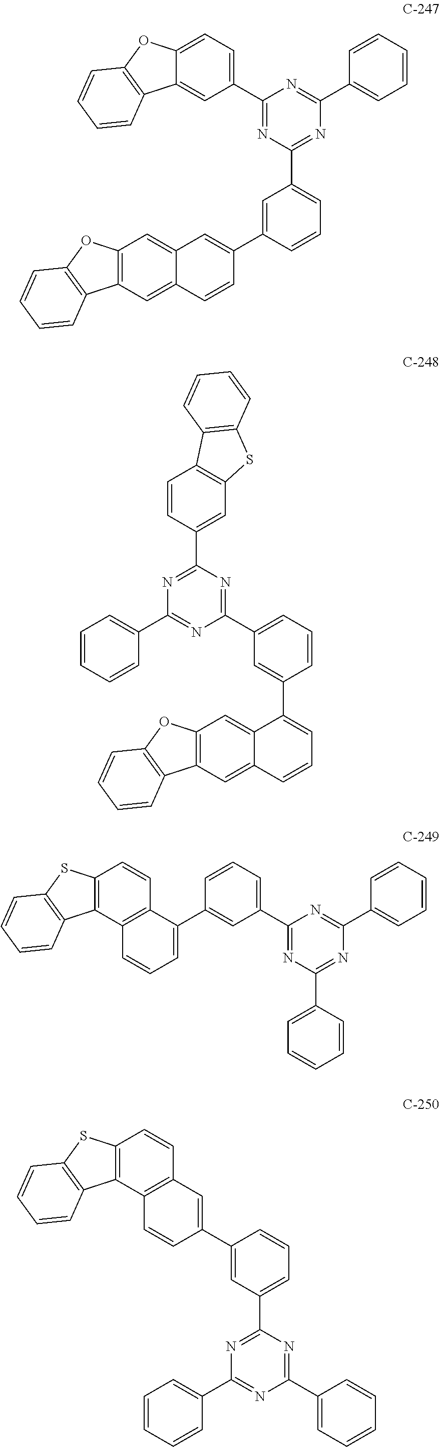 [SMILES: c1ccc(-c2nc(-c3cccc(-c4ccc5cc6c(cc5c4)oc4ccccc46)c3)nc(-c3ccc4oc5ccccc5c4c3)n2)cc1 ; c1ccc(-c2nc(-c3cccc(-c4cccc5cc6c(cc45)oc4ccccc46)c3)nc(-c3ccc4c(c3)sc3ccccc34)n2)cc1 ; c1ccc(-c2nc(-c3ccccc3)nc(-c3cccc(-c4cccc5c4ccc4sc6ccccc6c45)c3)n2)cc1 ; c1ccc(-c2nc(-c3ccccc3)nc(-c3cccc(-c4ccc5c(ccc6sc7ccccc7c65)c4)c3)n2)cc1]

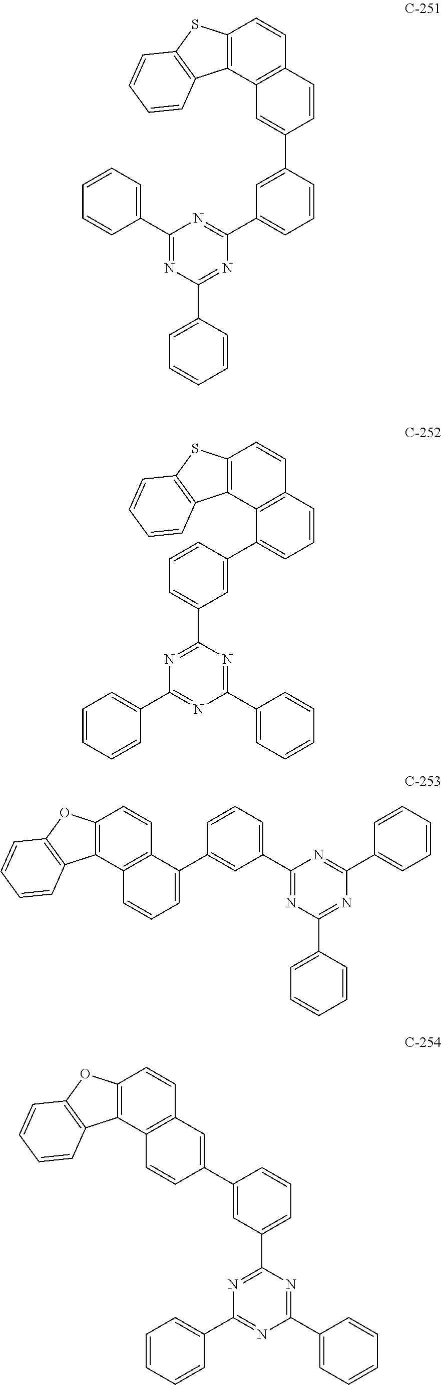 [SMILES: c1ccc(-c2nc(-c3ccccc3)nc(-c3cccc(-c4ccc5ccc6sc7ccccc7c6c5c4)c3)n2)cc1 ; c1ccc(-c2nc(-c3ccccc3)nc(-c3cccc(-c4cccc5ccc6sc7ccccc7c6c45)c3)n2)cc1 ; c1ccc(-c2nc(-c3ccccc3)nc(-c3cccc(-c4cccc5c4ccc4oc6ccccc6c45)c3)n2)cc1 ; c1ccc(-c2nc(-c3ccccc3)nc(-c3cccc(-c4ccc5c(ccc6oc7ccccc7c65)c4)c3)n2)cc1]

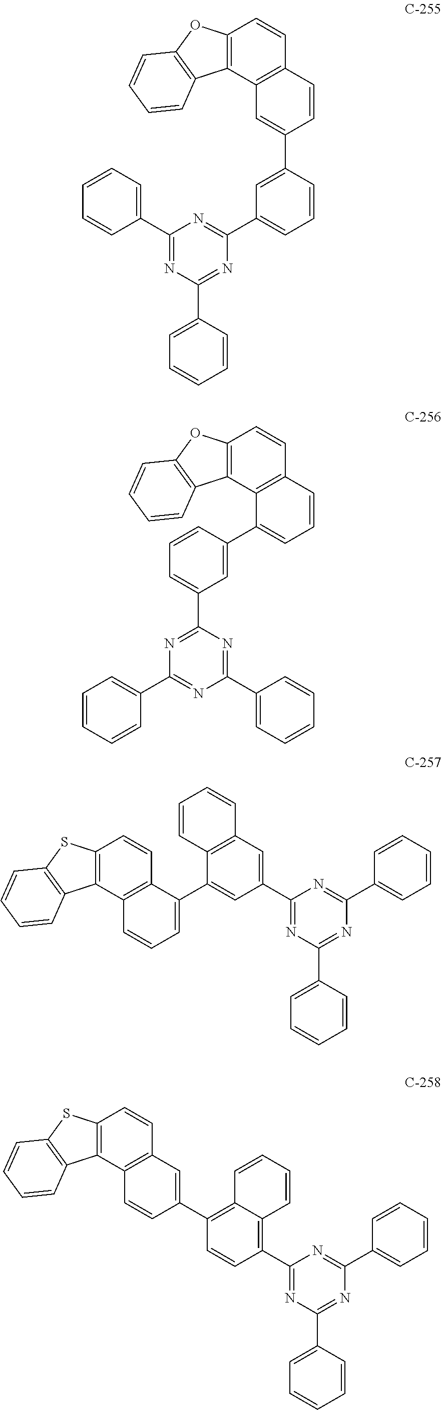 [SMILES: c1ccc(-c2nc(-c3ccccc3)nc(-c3cccc(-c4ccc5ccc6oc7ccccc7c6c5c4)c3)n2)cc1 ; c1ccc(-c2nc(-c3ccccc3)nc(-c3cccc(-c4cccc5ccc6oc7ccccc7c6c45)c3)n2)cc1 ; c1ccc(-c2nc(-c3ccccc3)nc(-c3cc(-c4cccc5c4ccc4sc6ccccc6c45)c4ccccc4c3)n2)cc1 ; c1ccc(-c2nc(-c3ccccc3)nc(-c3ccc(-c4ccc5c(ccc6sc7ccccc7c65)c4)c4ccccc34)n2)cc1]

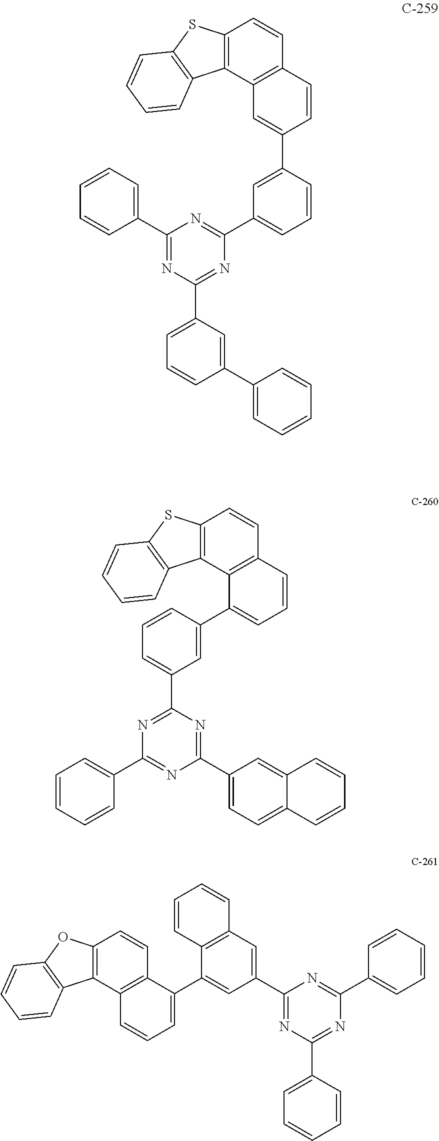 [SMILES: c1ccc(-c2cccc(-c3nc(-c4ccccc4)nc(-c4cccc(-c5ccc6ccc7sc8ccccc8c7c6c5)c4)n3)c2)cc1 ; c1ccc(-c2nc(-c3cccc(-c4cccc5ccc6sc7ccccc7c6c45)c3)nc(-c3ccc4ccccc4c3)n2)cc1 ; c1ccc(-c2nc(-c3ccccc3)nc(-c3cc(-c4cccc5c4ccc4oc6ccccc6c45)c4ccccc4c3)n2)cc1]

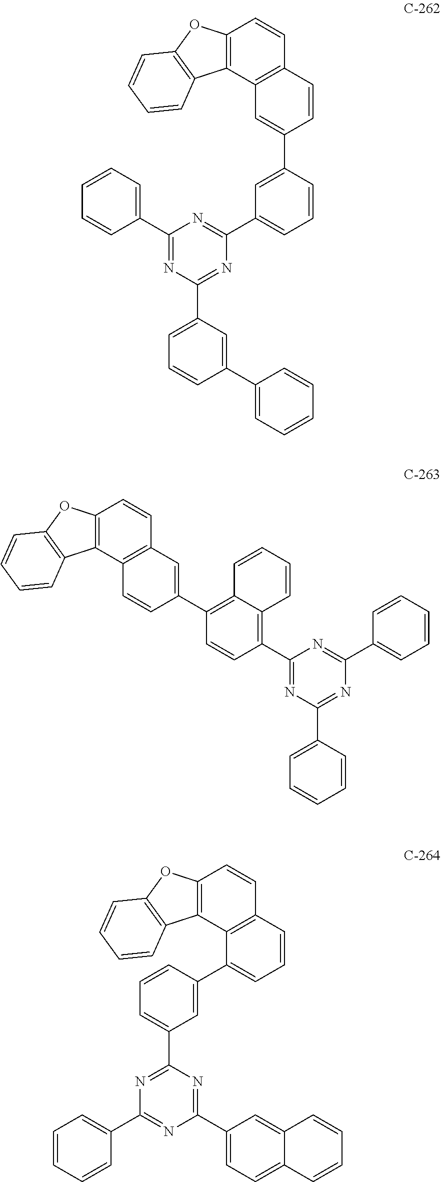 [SMILES: c1ccc(-c2cccc(-c3nc(-c4ccccc4)nc(-c4cccc(-c5ccc6ccc7oc8ccccc8c7c6c5)c4)n3)c2)cc1 ; c1ccc(-c2nc(-c3ccccc3)nc(-c3ccc(-c4ccc5c(ccc6oc7ccccc7c65)c4)c4ccccc34)n2)cc1 ; c1ccc(-c2nc(-c3cccc(-c4cccc5ccc6oc7ccccc7c6c45)c3)nc(-c3ccc4ccccc4c3)n2)cc1]

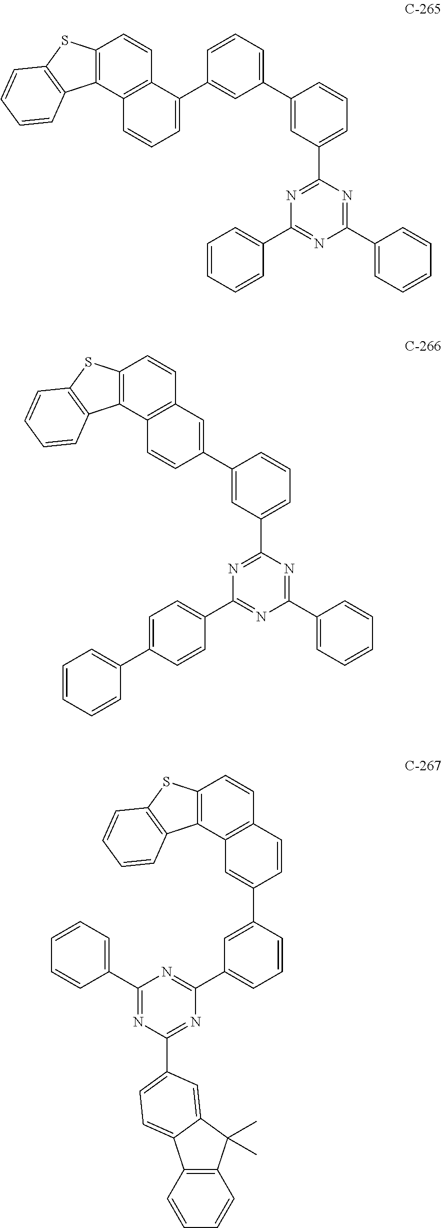 [SMILES: c1ccc(-c2nc(-c3ccccc3)nc(-c3cccc(-c4cccc(-c5cccc6c5ccc5sc7ccccc7c56)c4)c3)n2)cc1 ; c1ccc(-c2ccc(-c3nc(-c4ccccc4)nc(-c4cccc(-c5ccc6c(ccc7sc8ccccc8c76)c5)c4)n3)cc2)cc1 ; CC1(C)c2ccccc2-c2ccc(-c3nc(-c4ccccc4)nc(-c4cccc(-c5ccc6ccc7sc8ccccc8c7c6c5)c4)n3)cc21]

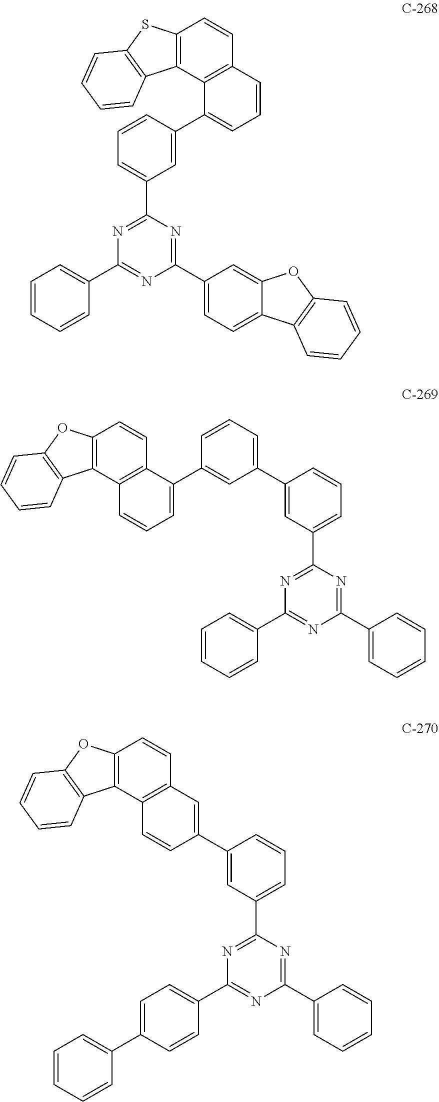 [SMILES: c1ccc(-c2nc(-c3cccc(-c4cccc5ccc6sc7ccccc7c6c45)c3)nc(-c3ccc4c(c3)oc3ccccc34)n2)cc1 ; c1ccc(-c2nc(-c3ccccc3)nc(-c3cccc(-c4cccc(-c5cccc6c5ccc5oc7ccccc7c56)c4)c3)n2)cc1 ; c1ccc(-c2ccc(-c3nc(-c4ccccc4)nc(-c4cccc(-c5ccc6c(ccc7oc8ccccc8c76)c5)c4)n3)cc2)cc1]

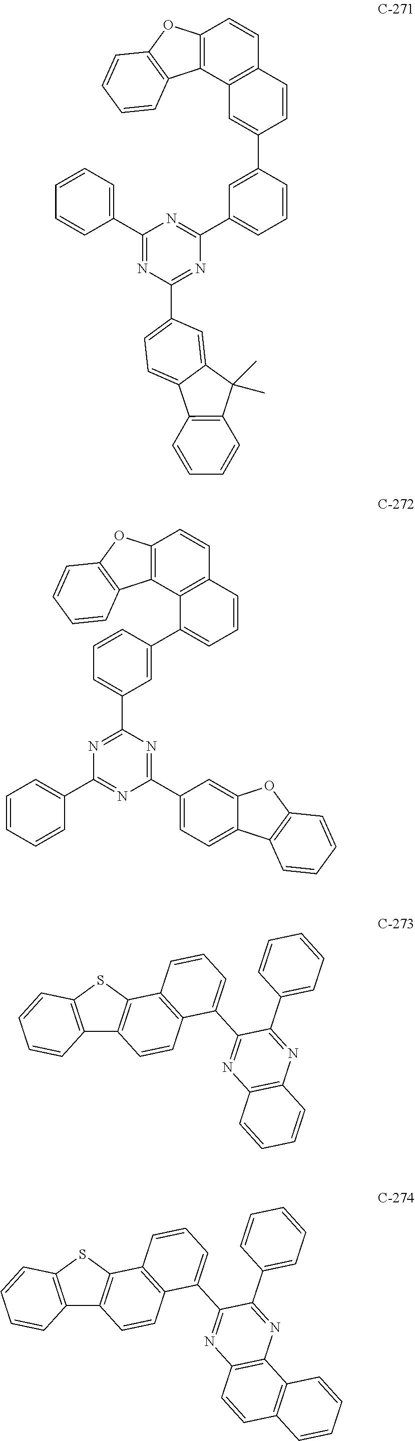 [SMILES: CC1(C)c2ccccc2-c2ccc(-c3nc(-c4ccccc4)nc(-c4cccc(-c5ccc6ccc7oc8ccccc8c7c6c5)c4)n3)cc21 ; c1ccc(-c2nc(-c3cccc(-c4cccc5ccc6oc7ccccc7c6c45)c3)nc(-c3ccc4c(c3)oc3ccccc34)n2)cc1 ; c1ccc(-c2nc3ccccc3nc2-c2cccc3c2ccc2c4ccccc4sc32)cc1 ; c1ccc(-c2nc3c(ccc4ccccc43)nc2-c2cccc3c2ccc2c4ccccc4sc32)cc1]

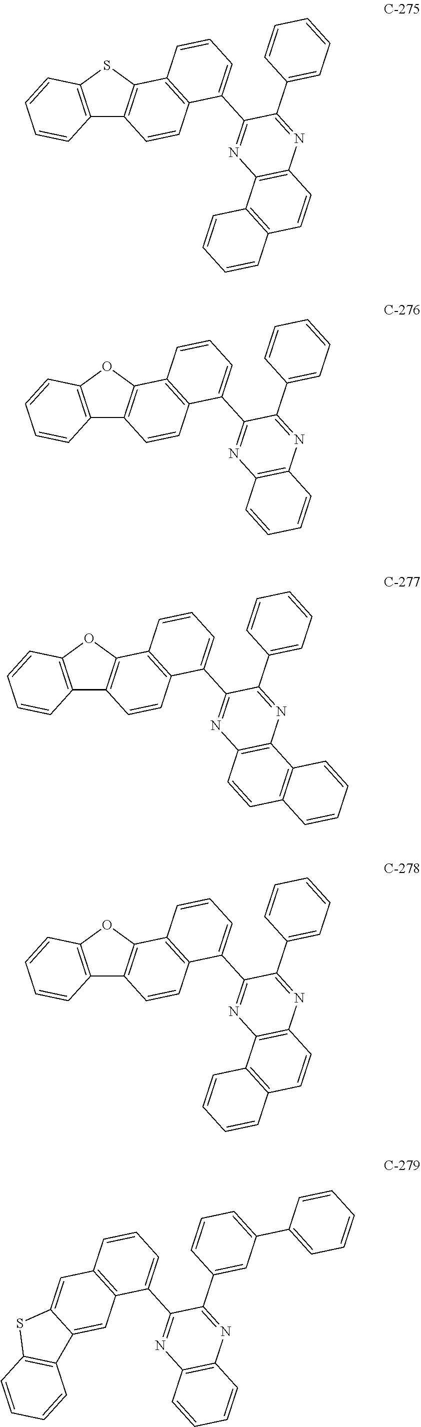 [SMILES: c1ccc(-c2nc3ccc4ccccc4c3nc2-c2cccc3c2ccc2c4ccccc4sc32)cc1 ; c1ccc(-c2nc3ccccc3nc2-c2cccc3c2ccc2c4ccccc4oc32)cc1 ; c1ccc(-c2nc3c(ccc4ccccc43)nc2-c2cccc3c2ccc2c4ccccc4oc32)cc1 ; c1ccc(-c2nc3ccc4ccccc4c3nc2-c2cccc3c2ccc2c4ccccc4oc32)cc1 ; c1ccc(-c2cccc(-c3nc4ccccc4nc3-c3cccc4cc5sc6ccccc6c5cc34)c2)cc1]

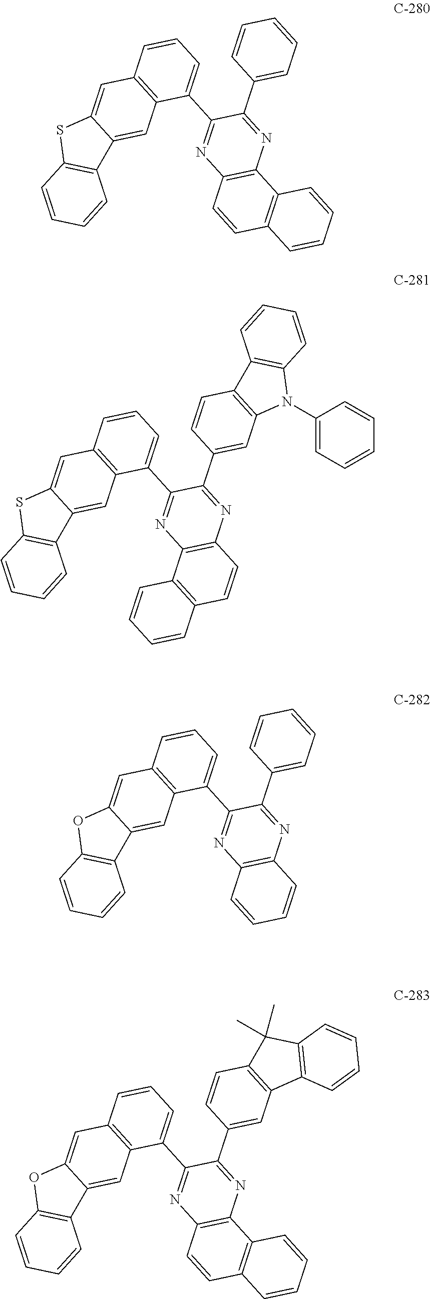 [SMILES: c1ccc(-c2nc3c(ccc4ccccc43)nc2-c2cccc3cc4sc5ccccc5c4cc23)cc1 ; c1ccc(-n2c3ccccc3c3ccc(-c4nc5ccc6ccccc6c5nc4-c4cccc5cc6sc7ccccc7c6cc45)cc32)cc1 ; c1ccc(-c2nc3ccccc3nc2-c2cccc3cc4oc5ccccc5c4cc23)cc1 ; CC1(C)c2ccccc2-c2cc(-c3nc4c(ccc5ccccc54)nc3-c3cccc4cc5oc6ccccc6c5cc34)ccc21]

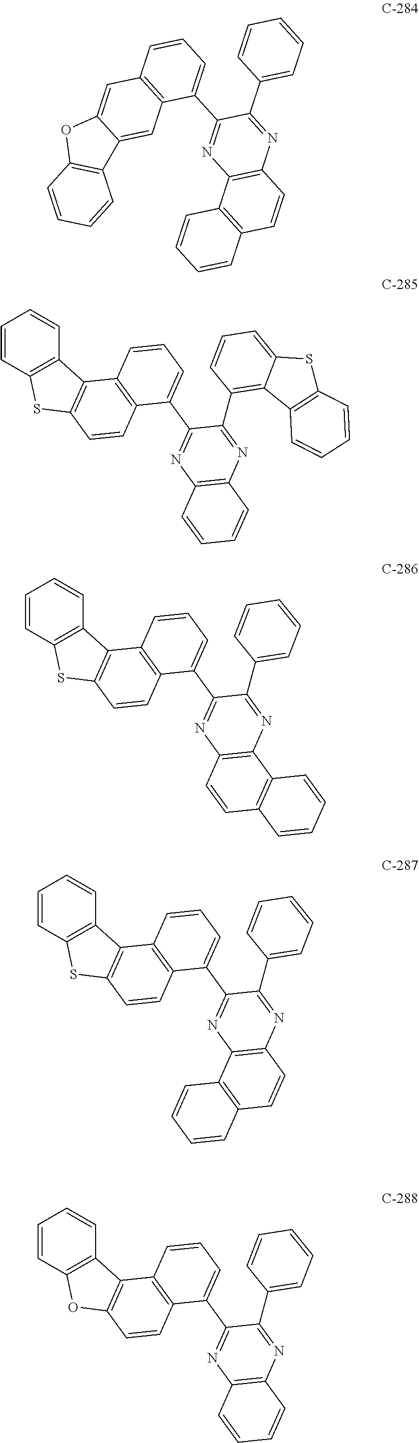 [SMILES: c1ccc(-c2nc3ccc4ccccc4c3nc2-c2cccc3cc4oc5ccccc5c4cc23)cc1 ; c1ccc2nc(-c3cccc4sc5ccccc5c34)c(-c3cccc4c3ccc3sc5ccccc5c34)nc2c1 ; c1ccc(-c2nc3c(ccc4ccccc43)nc2-c2cccc3c2ccc2sc4ccccc4c23)cc1 ; c1ccc(-c2nc3ccc4ccccc4c3nc2-c2cccc3c2ccc2sc4ccccc4c23)cc1 ; c1ccc(-c2nc3ccccc3nc2-c2cccc3c2ccc2oc4ccccc4c23)cc1]

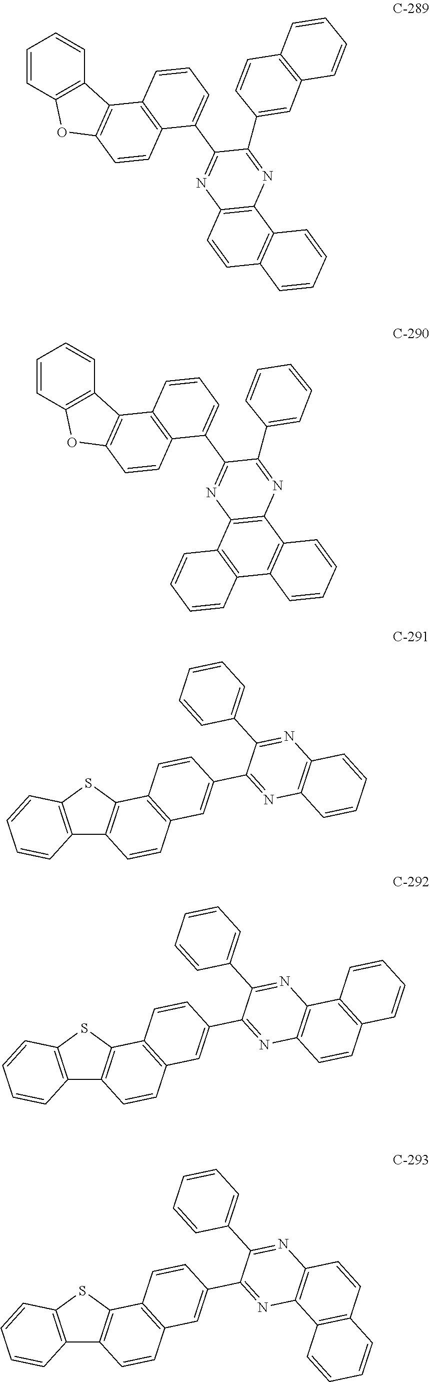 [SMILES: c1ccc2cc(-c3nc4c(ccc5ccccc54)nc3-c3cccc4c3ccc3oc5ccccc5c34)ccc2c1 ; c1ccc(-c2nc3c4ccccc4c4ccccc4c3nc2-c2cccc3c2ccc2oc4ccccc4c23)cc1 ; c1ccc(-c2nc3ccccc3nc2-c2ccc3c(ccc4c5ccccc5sc34)c2)cc1 ; c1ccc(-c2nc3c(ccc4ccccc43)nc2-c2ccc3c(ccc4c5ccccc5sc34)c2)cc1 ; c1ccc(-c2nc3ccc4ccccc4c3nc2-c2ccc3c(ccc4c5ccccc5sc34)c2)cc1]

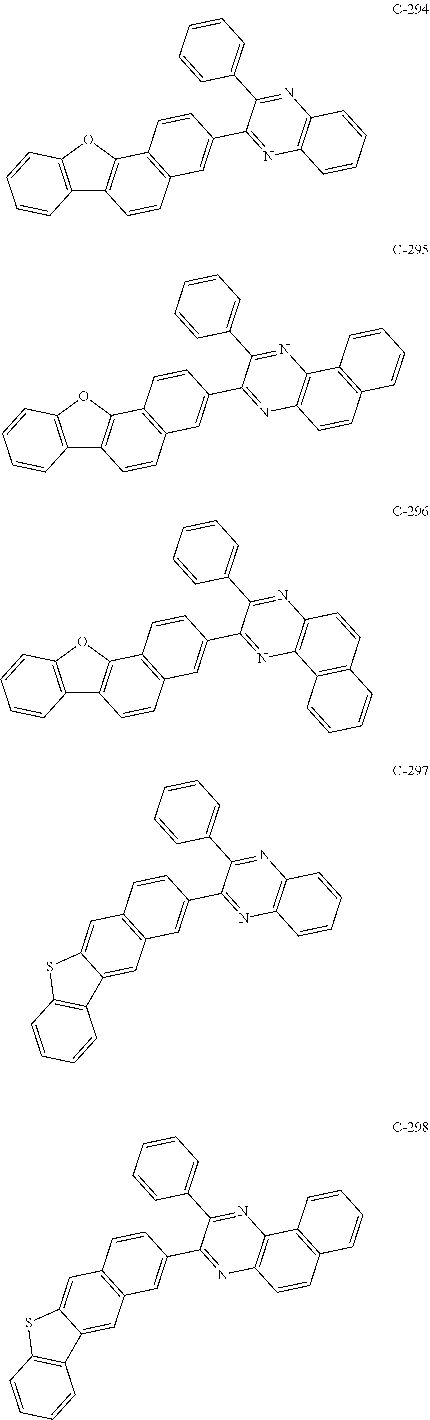 [SMILES: c1ccc(-c2nc3ccccc3nc2-c2ccc3c(ccc4c5ccccc5oc34)c2)cc1 ; c1ccc(-c2nc3c(ccc4ccccc43)nc2-c2ccc3c(ccc4c5ccccc5oc34)c2)cc1 ; c1ccc(-c2nc3ccc4ccccc4c3nc2-c2ccc3c(ccc4c5ccccc5oc34)c2)cc1 ; c1ccc(-c2nc3ccccc3nc2-c2ccc3cc4sc5ccccc5c4cc3c2)cc1 ; c1ccc(-c2nc3c(ccc4ccccc43)nc2-c2ccc3cc4sc5ccccc5c4cc3c2)cc1]

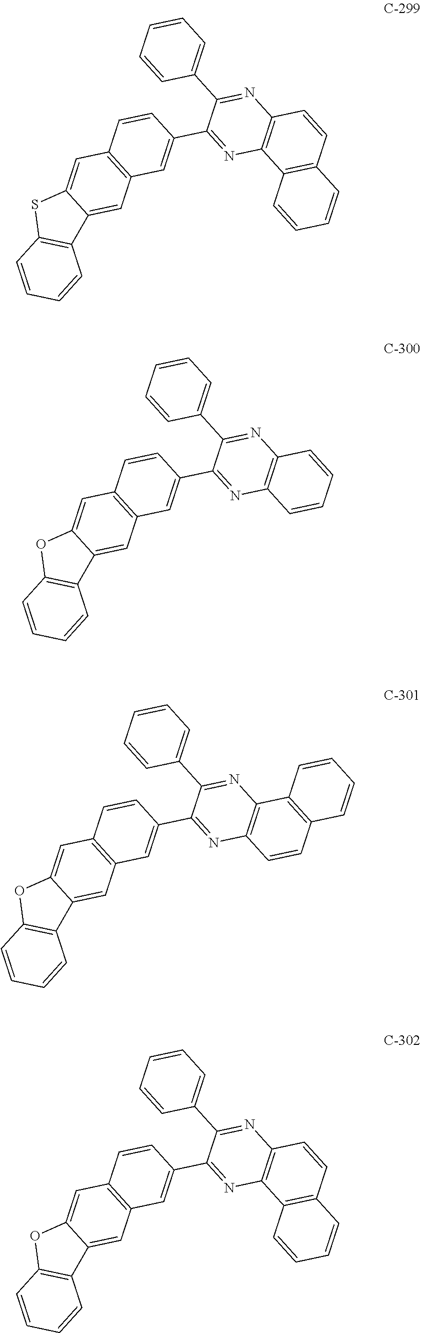 [SMILES: c1ccc(-c2nc3ccc4ccccc4c3nc2-c2ccc3cc4sc5ccccc5c4cc3c2)cc1 ; c1ccc(-c2nc3ccccc3nc2-c2ccc3cc4oc5ccccc5c4cc3c2)cc1 ; c1ccc(-c2nc3c(ccc4ccccc43)nc2-c2ccc3cc4oc5ccccc5c4cc3c2)cc1 ; c1ccc(-c2nc3ccc4ccccc4c3nc2-c2ccc3cc4oc5ccccc5c4cc3c2)cc1]

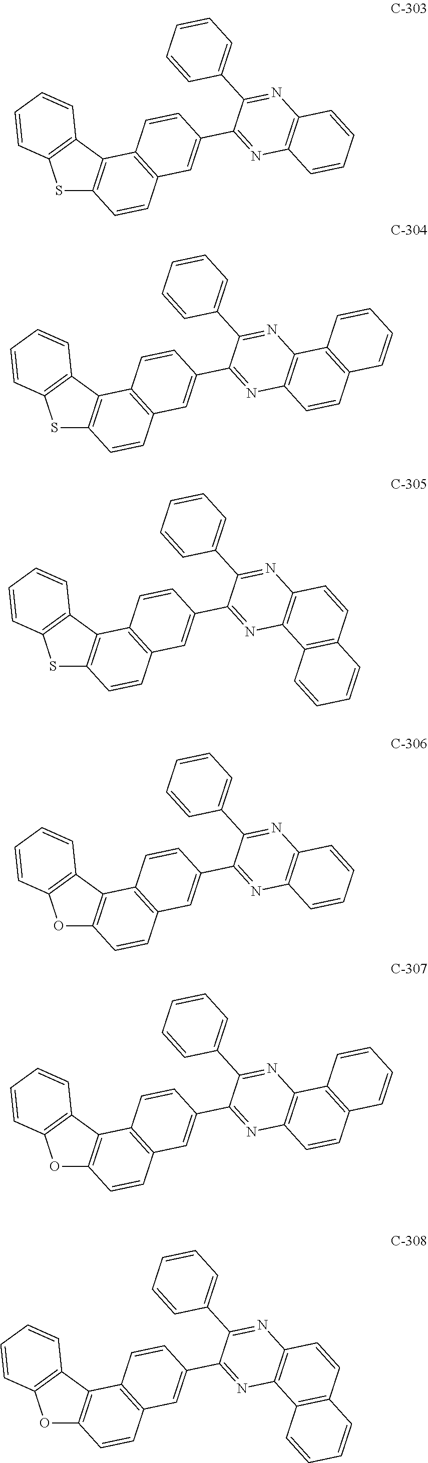 [SMILES: c1ccc(-c2nc3ccccc3nc2-c2ccc3c(ccc4sc5ccccc5c43)c2)cc1 ; c1ccc(-c2nc3c(ccc4ccccc43)nc2-c2ccc3c(ccc4sc5ccccc5c43)c2)cc1 ; c1ccc(-c2nc3ccc4ccccc4c3nc2-c2ccc3c(ccc4sc5ccccc5c43)c2)cc1 ; c1ccc(-c2nc3ccccc3nc2-c2ccc3c(ccc4oc5ccccc5c43)c2)cc1 ; c1ccc(-c2nc3c(ccc4ccccc43)nc2-c2ccc3c(ccc4oc5ccccc5c43)c2)cc1 ; c1ccc(-c2nc3ccc4ccccc4c3nc2-c2ccc3c(ccc4oc5ccccc5c43)c2)cc1]

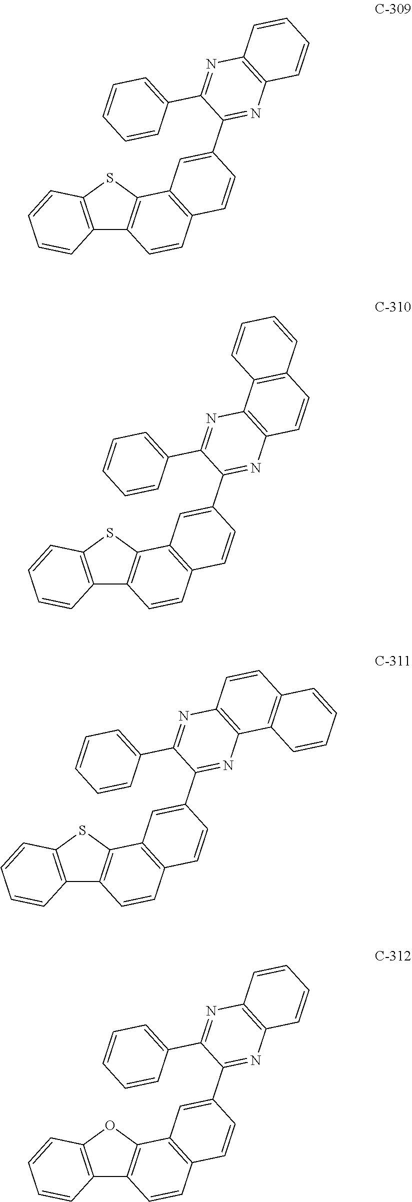 [SMILES: c1ccc(-c2nc3ccccc3nc2-c2ccc3ccc4c5ccccc5sc4c3c2)cc1 ; c1ccc(-c2nc3c(ccc4ccccc43)nc2-c2ccc3ccc4c5ccccc5sc4c3c2)cc1 ; c1ccc(-c2nc3ccc4ccccc4c3nc2-c2ccc3ccc4c5ccccc5sc4c3c2)cc1 ; c1ccc(-c2nc3ccccc3nc2-c2ccc3ccc4c5ccccc5oc4c3c2)cc1]

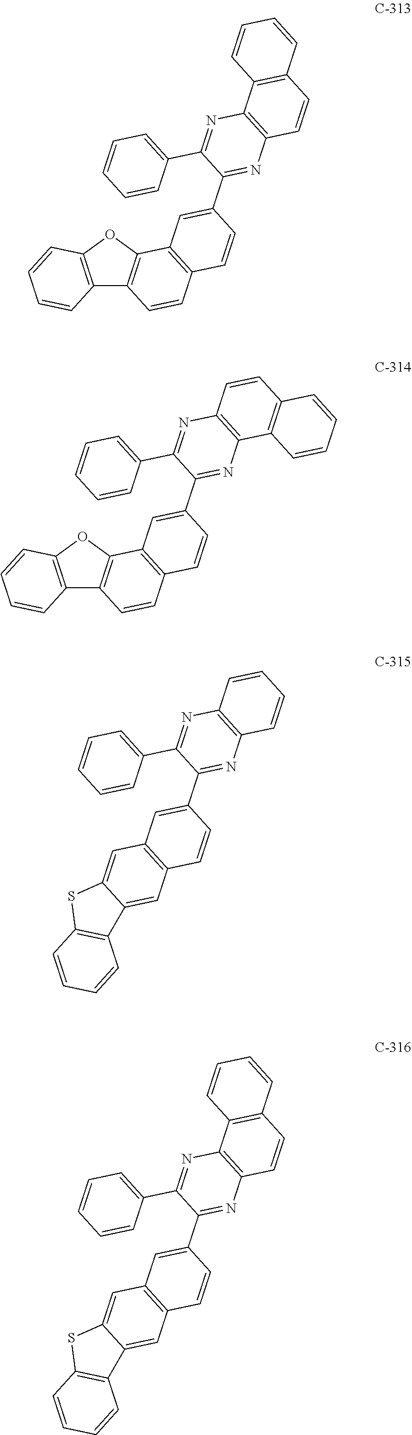 [SMILES: c1ccc(-c2nc3c(ccc4ccccc43)nc2-c2ccc3ccc4c5ccccc5oc4c3c2)cc1 ; c1ccc(-c2nc3ccc4ccccc4c3nc2-c2ccc3ccc4c5ccccc5oc4c3c2)cc1 ; c1ccc(-c2nc3ccccc3nc2-c2ccc3cc4c(cc3c2)sc2ccccc24)cc1 ; c1ccc(-c2nc3c(ccc4ccccc43)nc2-c2ccc3cc4c(cc3c2)sc2ccccc24)cc1]

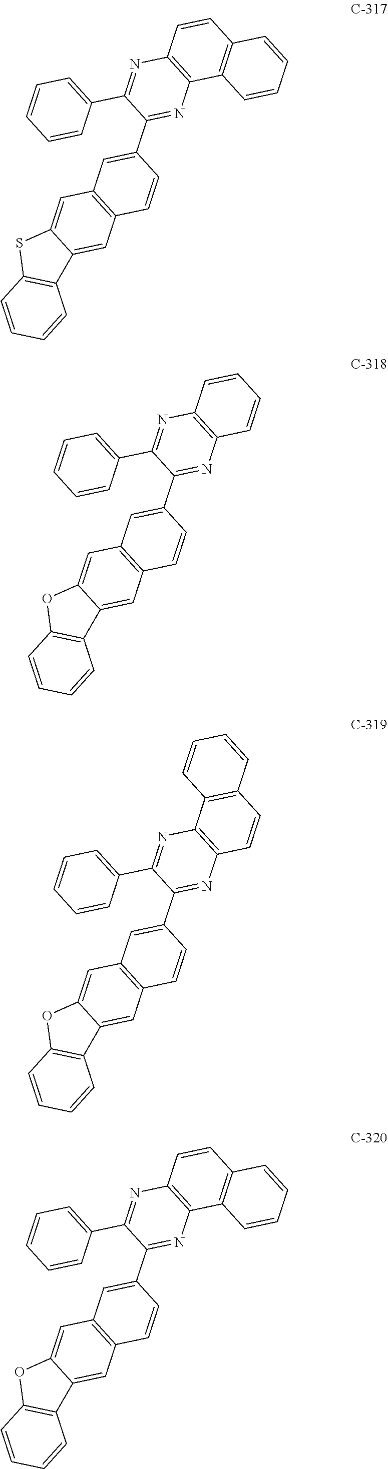 [SMILES: c1ccc(-c2nc3ccc4ccccc4c3nc2-c2ccc3cc4c(cc3c2)sc2ccccc24)cc1 ; c1ccc(-c2nc3ccccc3nc2-c2ccc3cc4c(cc3c2)oc2ccccc24)cc1 ; c1ccc(-c2nc3c(ccc4ccccc43)nc2-c2ccc3cc4c(cc3c2)oc2ccccc24)cc1 ; c1ccc(-c2nc3ccc4ccccc4c3nc2-c2ccc3cc4c(cc3c2)oc2ccccc24)cc1]

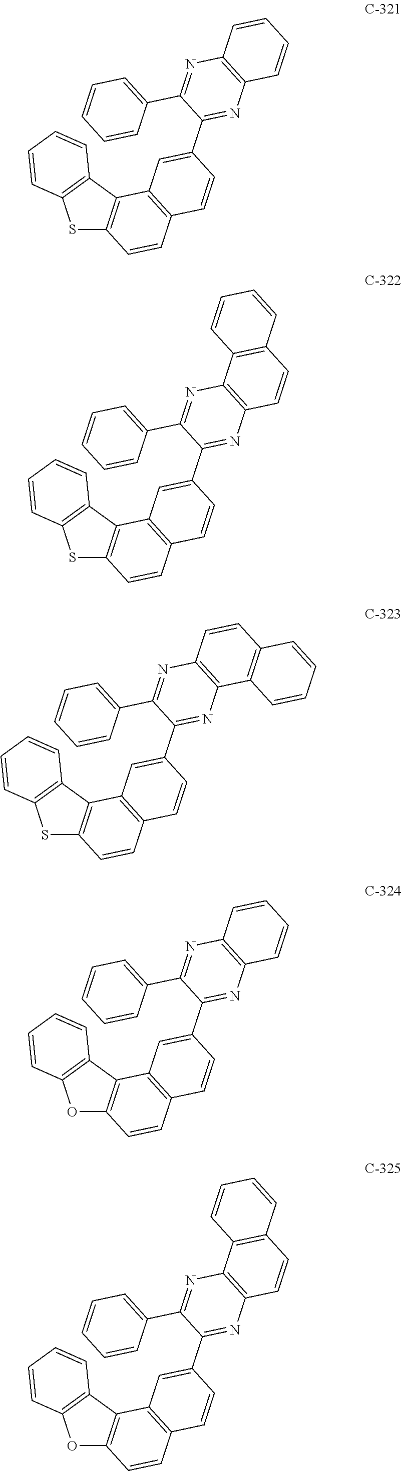 [SMILES: c1ccc(-c2nc3ccccc3nc2-c2ccc3ccc4sc5ccccc5c4c3c2)cc1 ; c1ccc(-c2nc3c(ccc4ccccc43)nc2-c2ccc3ccc4sc5ccccc5c4c3c2)cc1 ; c1ccc(-c2nc3ccc4ccccc4c3nc2-c2ccc3ccc4sc5ccccc5c4c3c2)cc1 ; c1ccc(-c2nc3ccccc3nc2-c2ccc3ccc4oc5ccccc5c4c3c2)cc1 ; c1ccc(-c2nc3c(ccc4ccccc43)nc2-c2ccc3ccc4oc5ccccc5c4c3c2)cc1]

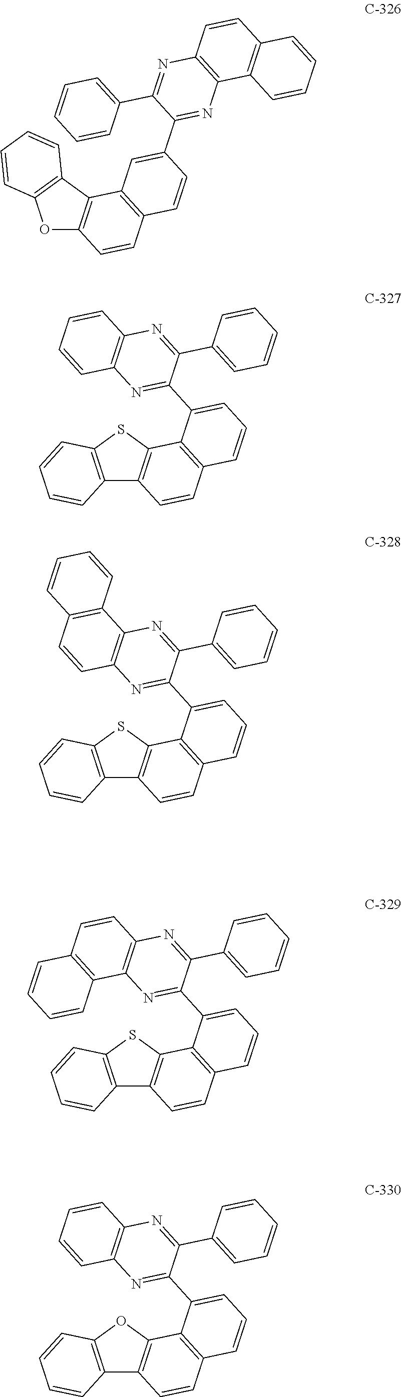 [SMILES: c1ccc(-c2nc3ccc4ccccc4c3nc2-c2ccc3ccc4oc5ccccc5c4c3c2)cc1 ; c1ccc(-c2nc3ccccc3nc2-c2cccc3ccc4c5ccccc5sc4c23)cc1 ; c1ccc(-c2nc3c(ccc4ccccc43)nc2-c2cccc3ccc4c5ccccc5sc4c23)cc1 ; c1ccc(-c2nc3ccc4ccccc4c3nc2-c2cccc3ccc4c5ccccc5sc4c23)cc1 ; c1ccc(-c2nc3ccccc3nc2-c2cccc3ccc4c5ccccc5oc4c23)cc1]

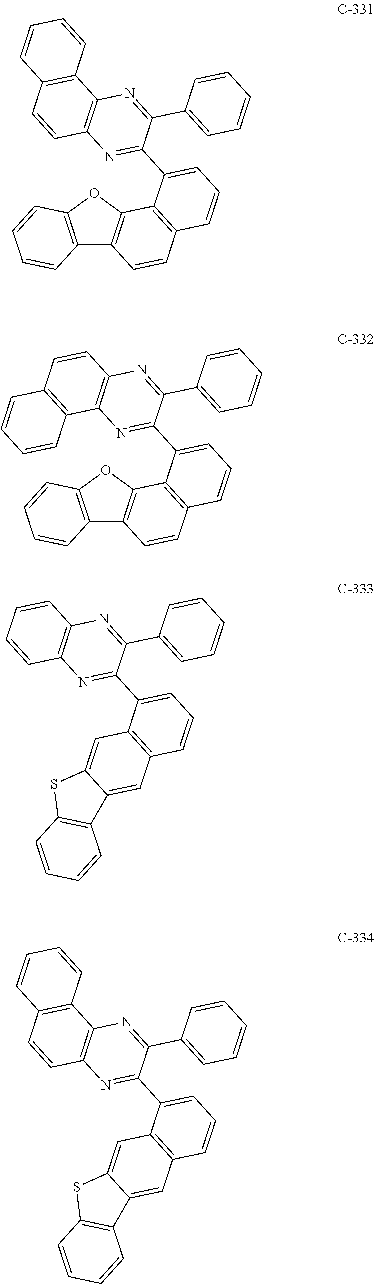 [SMILES: c1ccc(-c2nc3c(ccc4ccccc43)nc2-c2cccc3ccc4c5ccccc5oc4c23)cc1 ; c1ccc(-c2nc3ccc4ccccc4c3nc2-c2cccc3ccc4c5ccccc5oc4c23)cc1 ; c1ccc(-c2nc3ccccc3nc2-c2cccc3cc4c(cc23)sc2ccccc24)cc1 ; c1ccc(-c2nc3c(ccc4ccccc43)nc2-c2cccc3cc4c(cc23)sc2ccccc24)cc1]

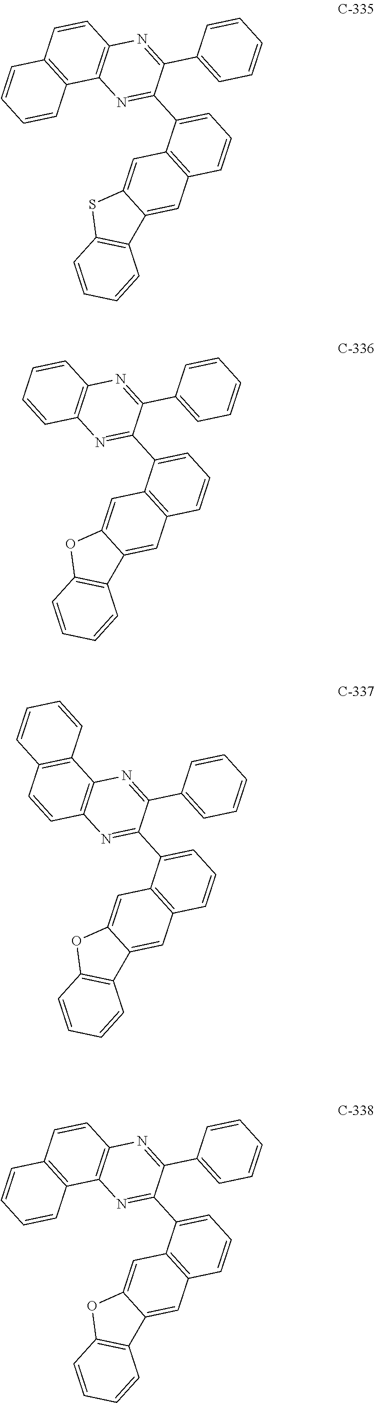 [SMILES: c1ccc(-c2nc3ccc4ccccc4c3nc2-c2cccc3cc4c(cc23)sc2ccccc24)cc1 ; c1ccc(-c2nc3ccccc3nc2-c2cccc3cc4c(cc23)oc2ccccc24)cc1 ; c1ccc(-c2nc3c(ccc4ccccc43)nc2-c2cccc3cc4c(cc23)oc2ccccc24)cc1 ; c1ccc(-c2nc3ccc4ccccc4c3nc2-c2cccc3cc4c(cc23)oc2ccccc24)cc1]

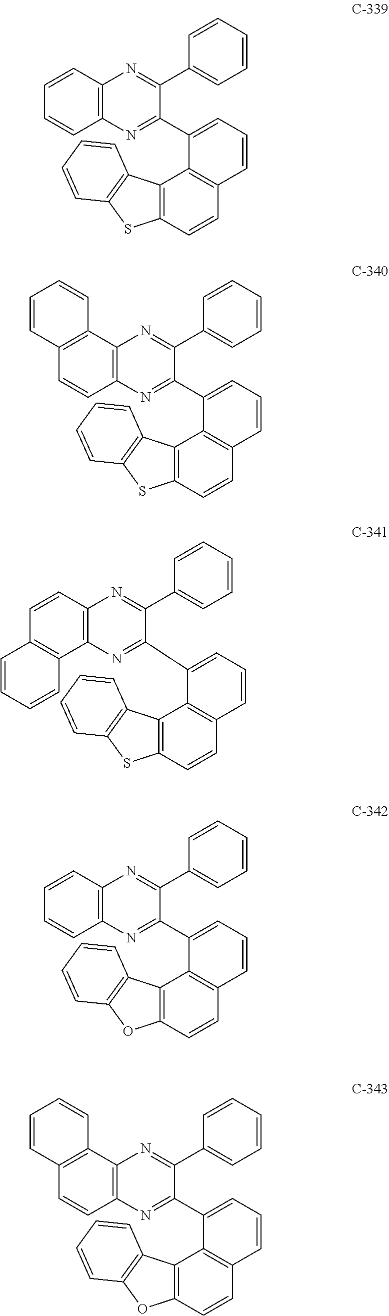

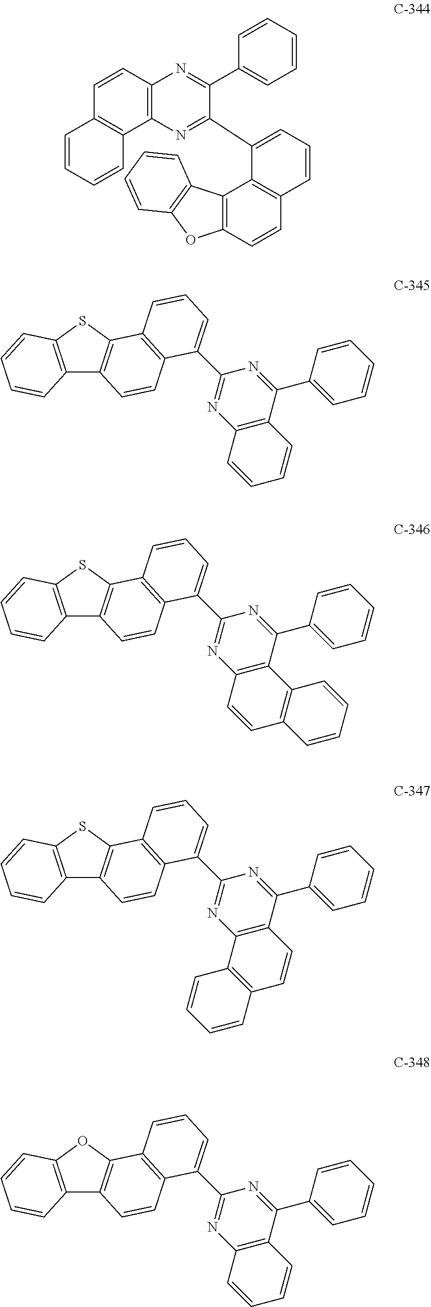 [SMILES: c1ccc(-c2nc3ccc4ccccc4c3nc2-c2cccc3ccc4oc5ccccc5c4c23)cc1 ; c1ccc(-c2nc(-c3cccc4c3ccc3c5ccccc5sc43)nc3ccccc23)cc1 ; c1ccc(-c2nc(-c3cccc4c3ccc3c5ccccc5sc43)nc3ccc4ccccc4c23)cc1 ; c1ccc(-c2nc(-c3cccc4c3ccc3c5ccccc5sc43)nc3c2ccc2ccccc23)cc1 ; c1ccc(-c2nc(-c3cccc4c3ccc3c5ccccc5oc43)nc3ccccc23)cc1]

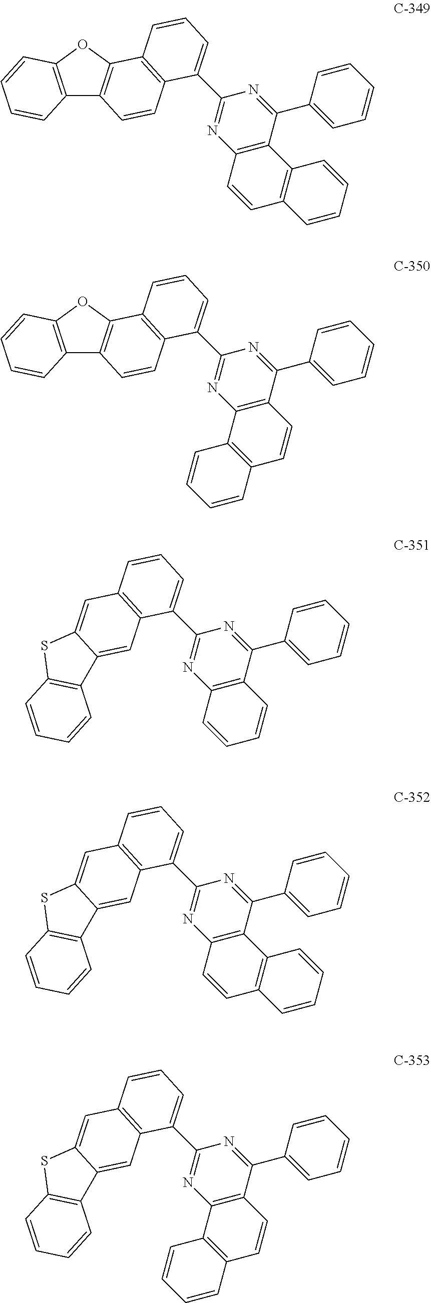 [SMILES: c1ccc(-c2nc(-c3cccc4c3ccc3c5ccccc5oc43)nc3ccc4ccccc4c23)cc1 ; c1ccc(-c2nc(-c3cccc4c3ccc3c5ccccc5oc43)nc3c2ccc2ccccc23)cc1 ; c1ccc(-c2nc(-c3cccc4cc5sc6ccccc6c5cc34)nc3ccccc23)cc1 ; c1ccc(-c2nc(-c3cccc4cc5sc6ccccc6c5cc34)nc3ccc4ccccc4c23)cc1 ; c1ccc(-c2nc(-c3cccc4cc5sc6ccccc6c5cc34)nc3c2ccc2ccccc23)cc1]

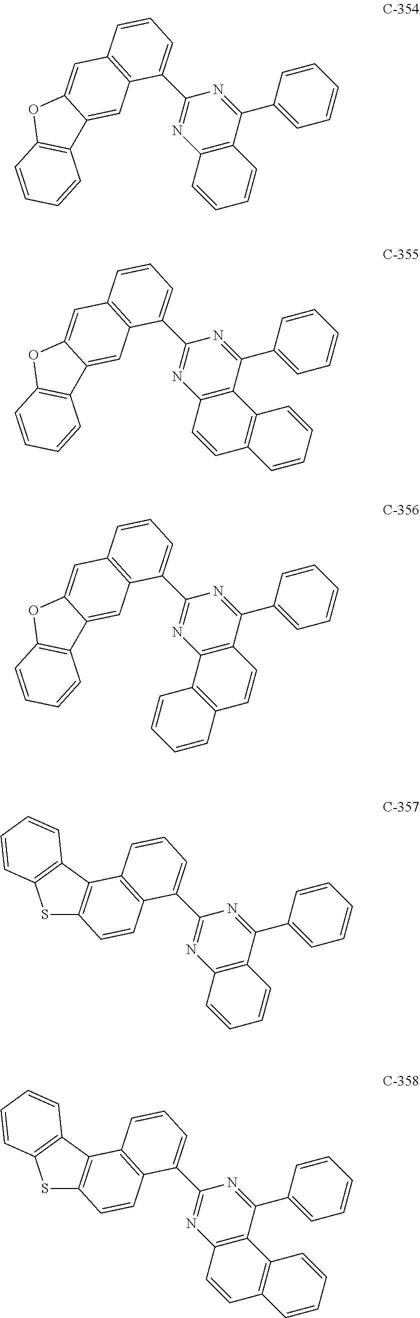 [SMILES: c1ccc(-c2nc(-c3cccc4cc5oc6ccccc6c5cc34)nc3ccccc23)cc1 ; c1ccc(-c2nc(-c3cccc4cc5oc6ccccc6c5cc34)nc3ccc4ccccc4c23)cc1 ; c1ccc(-c2nc(-c3cccc4cc5oc6ccccc6c5cc34)nc3c2ccc2ccccc23)cc1 ; c1ccc(-c2nc(-c3cccc4c3ccc3sc5ccccc5c34)nc3ccccc23)cc1 ; c1ccc(-c2nc(-c3cccc4c3ccc3sc5ccccc5c34)nc3ccc4ccccc4c23)cc1]

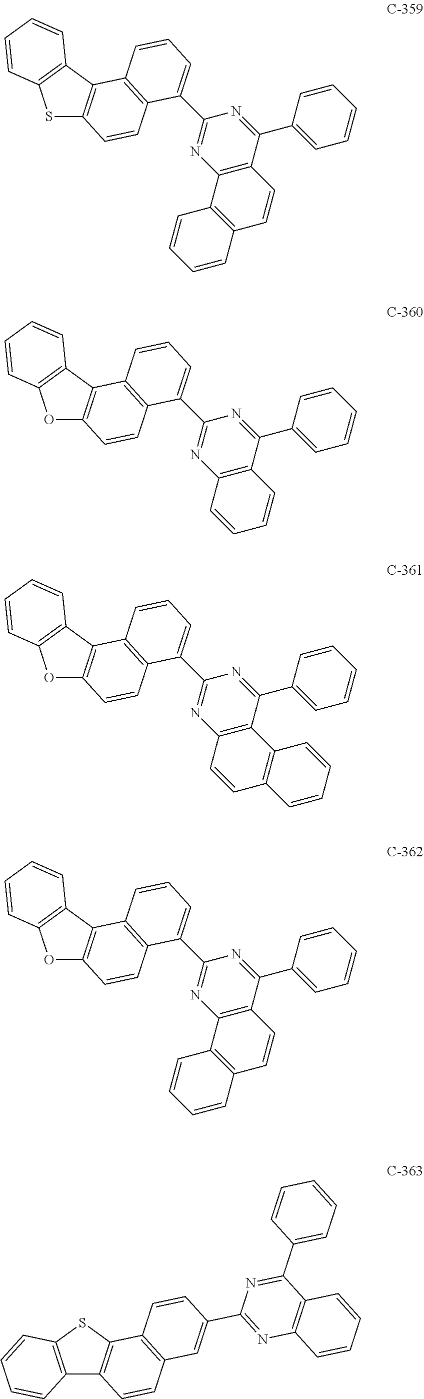 [SMILES: c1ccc(-c2nc(-c3cccc4c3ccc3sc5ccccc5c34)nc3c2ccc2ccccc23)cc1 ; c1ccc(-c2nc(-c3cccc4c3ccc3oc5ccccc5c34)nc3ccccc23)cc1 ; c1ccc(-c2nc(-c3cccc4c3ccc3oc5ccccc5c34)nc3ccc4ccccc4c23)cc1 ; c1ccc(-c2nc(-c3cccc4c3ccc3oc5ccccc5c34)nc3c2ccc2ccccc23)cc1 ; c1ccc(-c2nc(-c3ccc4c(ccc5c6ccccc6sc45)c3)nc3ccccc23)cc1]

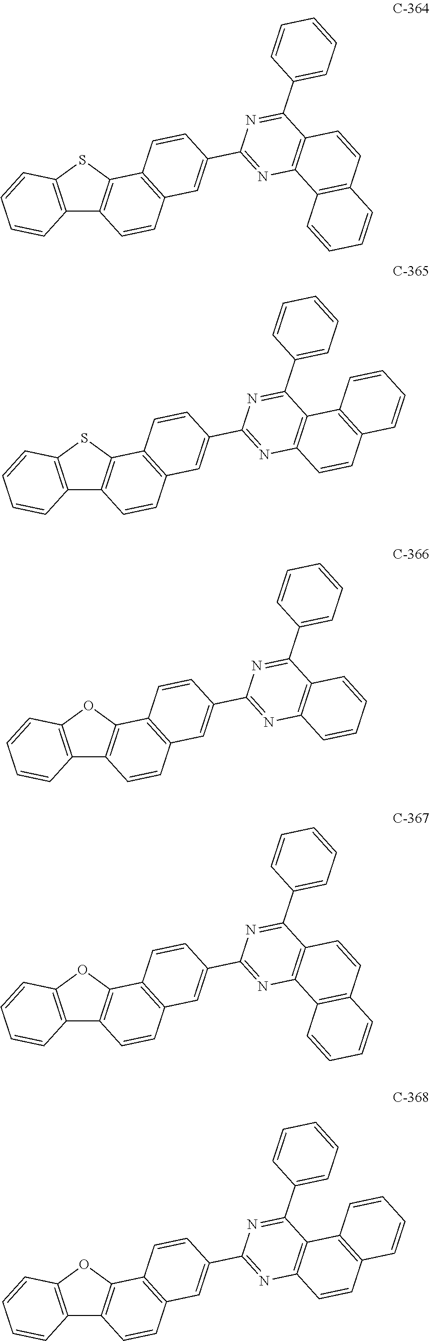 [SMILES: c1ccc(-c2nc(-c3ccc4c(ccc5c6ccccc6sc45)c3)nc3c2ccc2ccccc23)cc1 ; c1ccc(-c2nc(-c3ccc4c(ccc5c6ccccc6sc45)c3)nc3ccc4ccccc4c23)cc1 ; c1ccc(-c2nc(-c3ccc4c(ccc5c6ccccc6oc45)c3)nc3ccccc23)cc1 ; c1ccc(-c2nc(-c3ccc4c(ccc5c6ccccc6oc45)c3)nc3c2ccc2ccccc23)cc1 ; c1ccc(-c2nc(-c3ccc4c(ccc5c6ccccc6oc45)c3)nc3ccc4ccccc4c23)cc1]

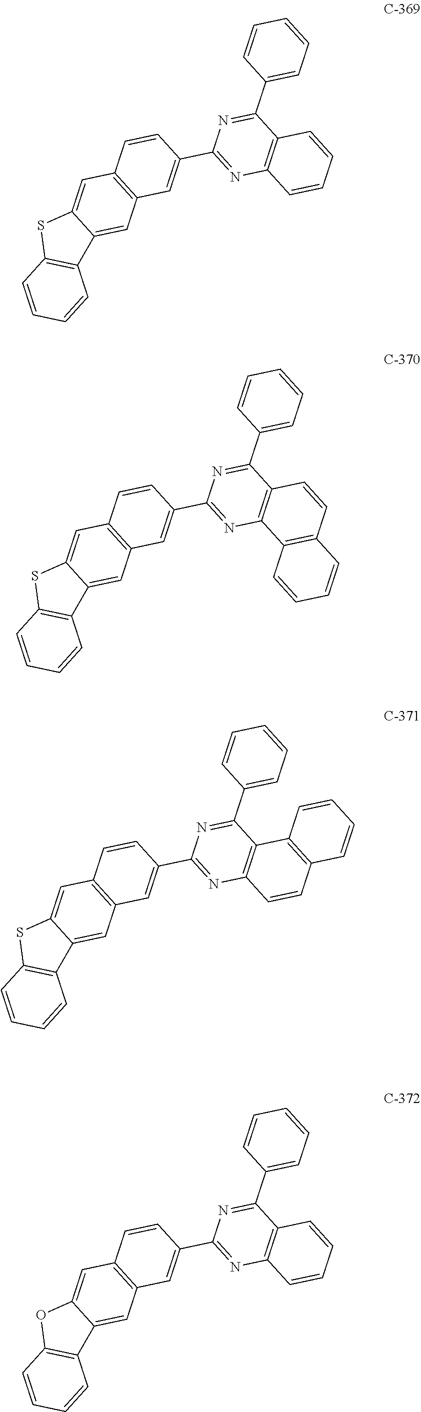 [SMILES: c1ccc(-c2nc(-c3ccc4cc5sc6ccccc6c5cc4c3)nc3ccccc23)cc1 ; c1ccc(-c2nc(-c3ccc4cc5sc6ccccc6c5cc4c3)nc3c2ccc2ccccc23)cc1 ; c1ccc(-c2nc(-c3ccc4cc5sc6ccccc6c5cc4c3)nc3ccc4ccccc4c23)cc1 ; c1ccc(-c2nc(-c3ccc4cc5oc6ccccc6c5cc4c3)nc3ccccc23)cc1]

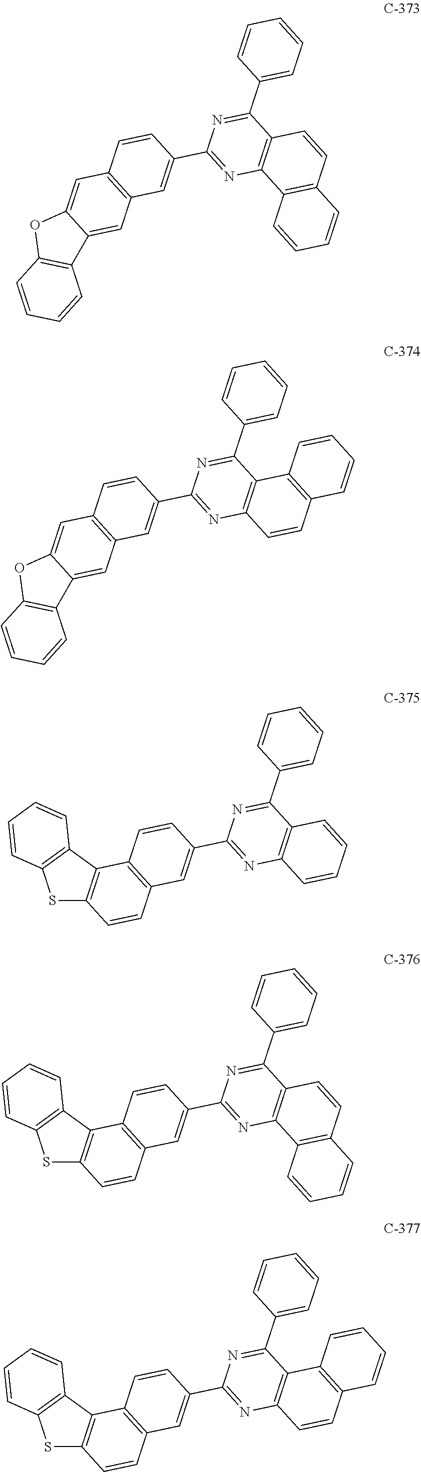 [SMILES: c1ccc(-c2nc(-c3ccc4cc5oc6ccccc6c5cc4c3)nc3c2ccc2ccccc23)cc1 ; c1ccc(-c2nc(-c3ccc4cc5oc6ccccc6c5cc4c3)nc3ccc4ccccc4c23)cc1 ; c1ccc(-c2nc(-c3ccc4c(ccc5sc6ccccc6c54)c3)nc3ccccc23)cc1 ; c1ccc(-c2nc(-c3ccc4c(ccc5sc6ccccc6c54)c3)nc3c2ccc2ccccc23)cc1 ; c1ccc(-c2nc(-c3ccc4c(ccc5sc6ccccc6c54)c3)nc3ccc4ccccc4c23)cc1]

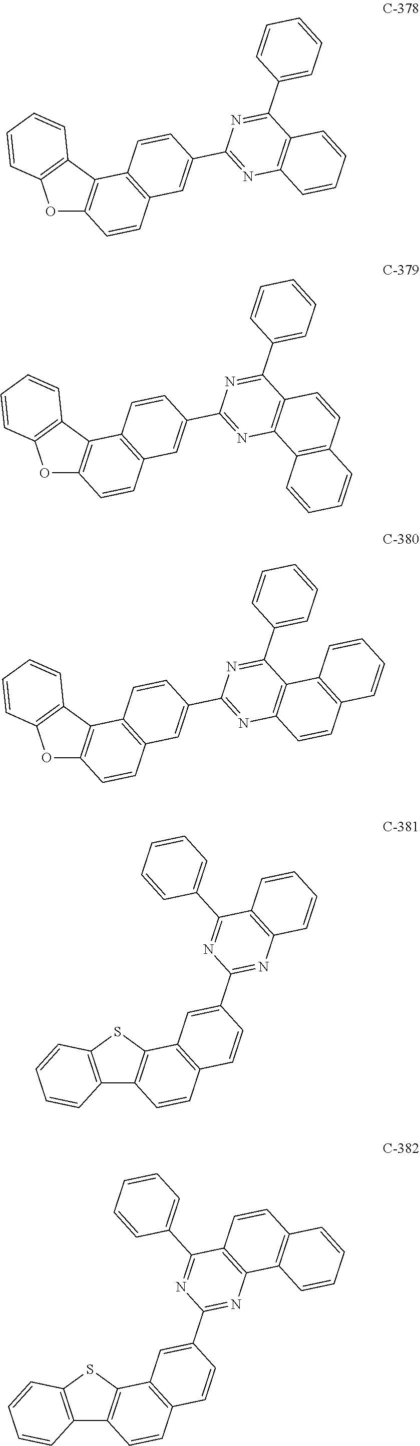 [SMILES: c1ccc(-c2nc(-c3ccc4c(ccc5oc6ccccc6c54)c3)nc3ccccc23)cc1 ; c1ccc(-c2nc(-c3ccc4c(ccc5oc6ccccc6c54)c3)nc3c2ccc2ccccc23)cc1 ; c1ccc(-c2nc(-c3ccc4c(ccc5oc6ccccc6c54)c3)nc3ccc4ccccc4c23)cc1 ; c1ccc(-c2nc(-c3ccc4ccc5c6ccccc6sc5c4c3)nc3ccccc23)cc1 ; c1ccc(-c2nc(-c3ccc4ccc5c6ccccc6sc5c4c3)nc3c2ccc2ccccc23)cc1]

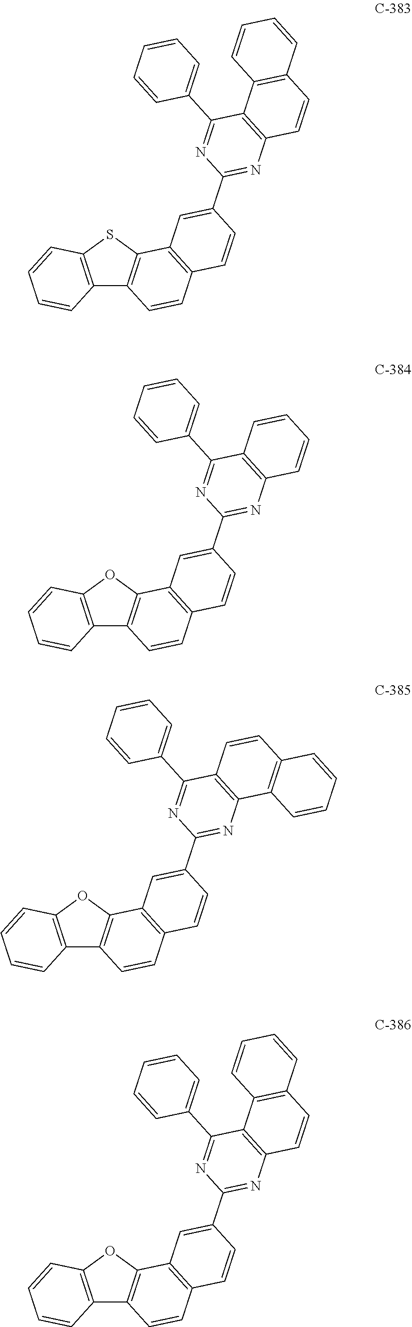 [SMILES: c1ccc(-c2nc(-c3ccc4ccc5c6ccccc6sc5c4c3)nc3ccc4ccccc4c23)cc1 ; c1ccc(-c2nc(-c3ccc4ccc5c6ccccc6oc5c4c3)nc3ccccc23)cc1 ; c1ccc(-c2nc(-c3ccc4ccc5c6ccccc6oc5c4c3)nc3c2ccc2ccccc23)cc1 ; c1ccc(-c2nc(-c3ccc4ccc5c6ccccc6oc5c4c3)nc3ccc4ccccc4c23)cc1]

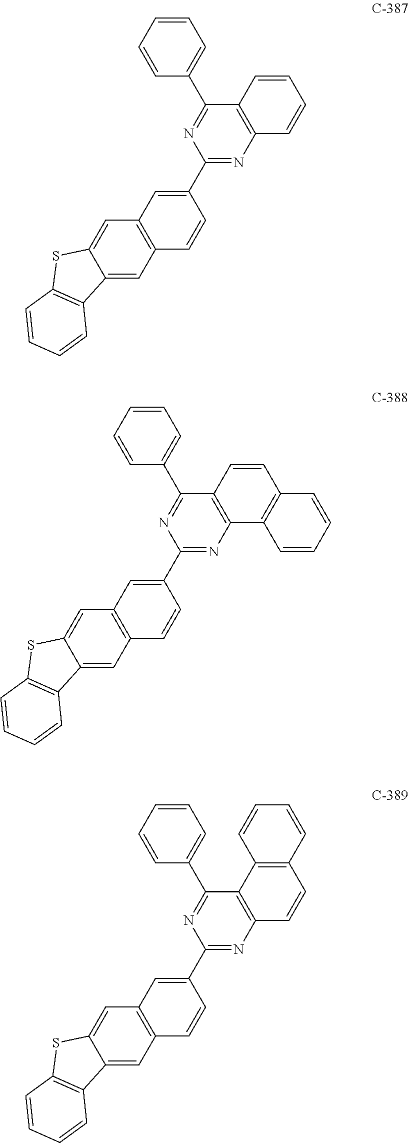 [SMILES: c1ccc(-c2nc(-c3ccc4cc5c(cc4c3)sc3ccccc35)nc3ccccc23)cc1 ; c1ccc(-c2nc(-c3ccc4cc5c(cc4c3)sc3ccccc35)nc3c2ccc2ccccc23)cc1 ; c1ccc(-c2nc(-c3ccc4cc5c(cc4c3)sc3ccccc35)nc3ccc4ccccc4c23)cc1]

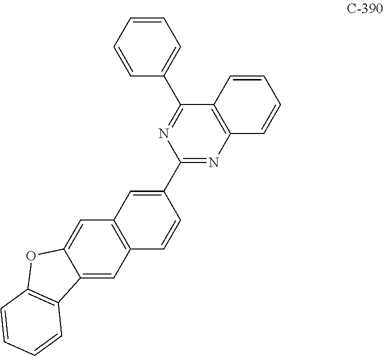 [SMILES: c1ccc(-c2nc(-c3ccc4cc5c(cc4c3)oc3ccccc35)nc3ccccc23)cc1]

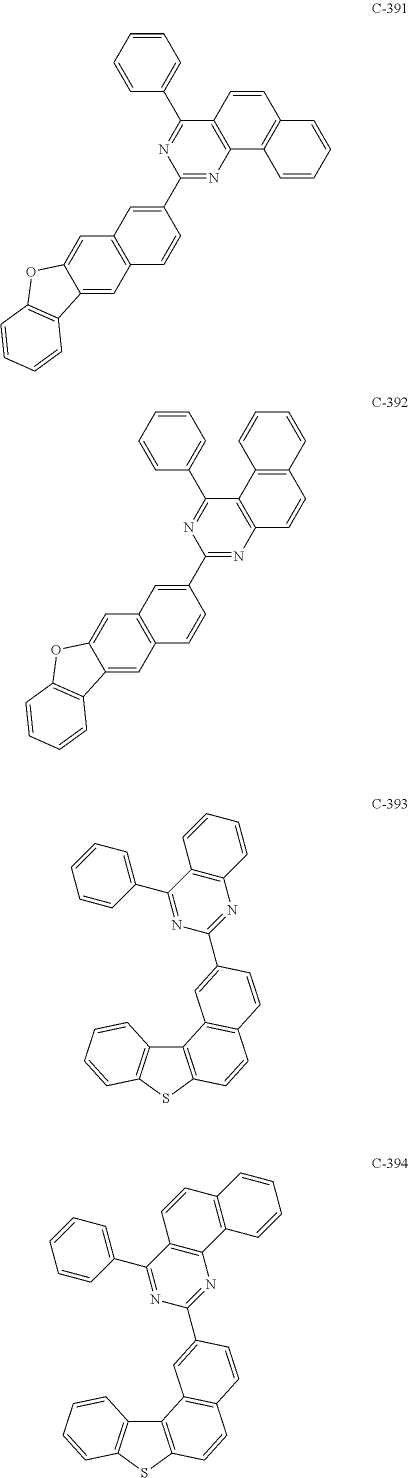 [SMILES: c1ccc(-c2nc(-c3ccc4cc5c(cc4c3)oc3ccccc35)nc3c2ccc2ccccc23)cc1 ; c1ccc(-c2nc(-c3ccc4cc5c(cc4c3)oc3ccccc35)nc3ccc4ccccc4c23)cc1 ; c1ccc(-c2nc(-c3ccc4ccc5sc6ccccc6c5c4c3)nc3ccccc23)cc1 ; c1ccc(-c2nc(-c3ccc4ccc5sc6ccccc6c5c4c3)nc3c2ccc2ccccc23)cc1]

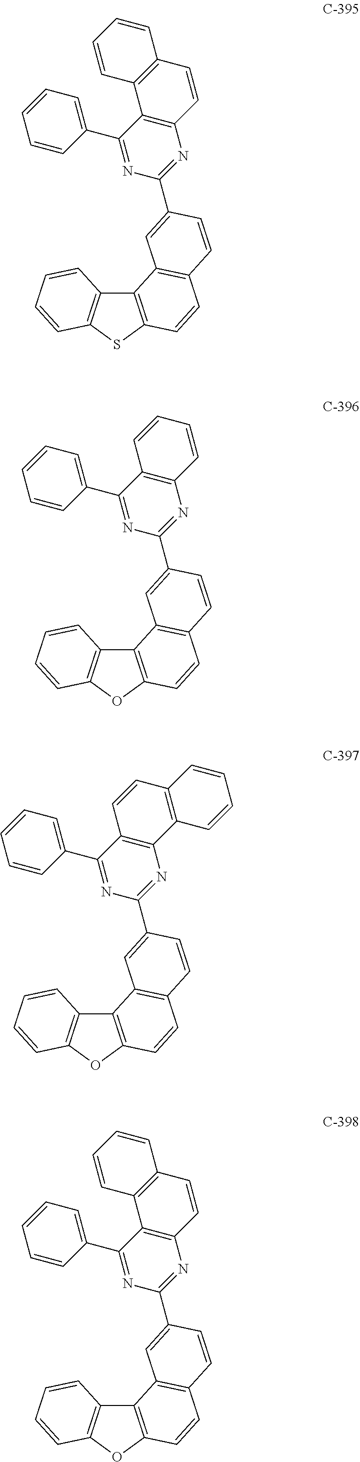 [SMILES: c1ccc(-c2nc(-c3ccc4ccc5sc6ccccc6c5c4c3)nc3ccc4ccccc4c23)cc1 ; c1ccc(-c2nc(-c3ccc4ccc5oc6ccccc6c5c4c3)nc3ccccc23)cc1 ; c1ccc(-c2nc(-c3ccc4ccc5oc6ccccc6c5c4c3)nc3c2ccc2ccccc23)cc1 ; c1ccc(-c2nc(-c3ccc4ccc5oc6ccccc6c5c4c3)nc3ccc4ccccc4c23)cc1]

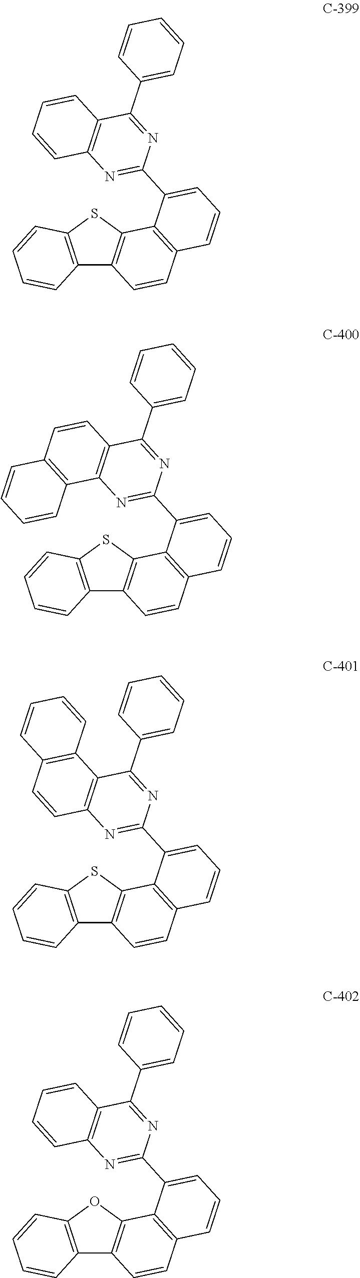 [SMILES: c1ccc(-c2nc(-c3cccc4ccc5c6ccccc6sc5c34)nc3ccccc23)cc1 ; c1ccc(-c2nc(-c3cccc4ccc5c6ccccc6sc5c34)nc3c2ccc2ccccc23)cc1 ; c1ccc(-c2nc(-c3cccc4ccc5c6ccccc6sc5c34)nc3ccc4ccccc4c23)cc1 ; c1ccc(-c2nc(-c3cccc4ccc5c6ccccc6oc5c34)nc3ccccc23)cc1]

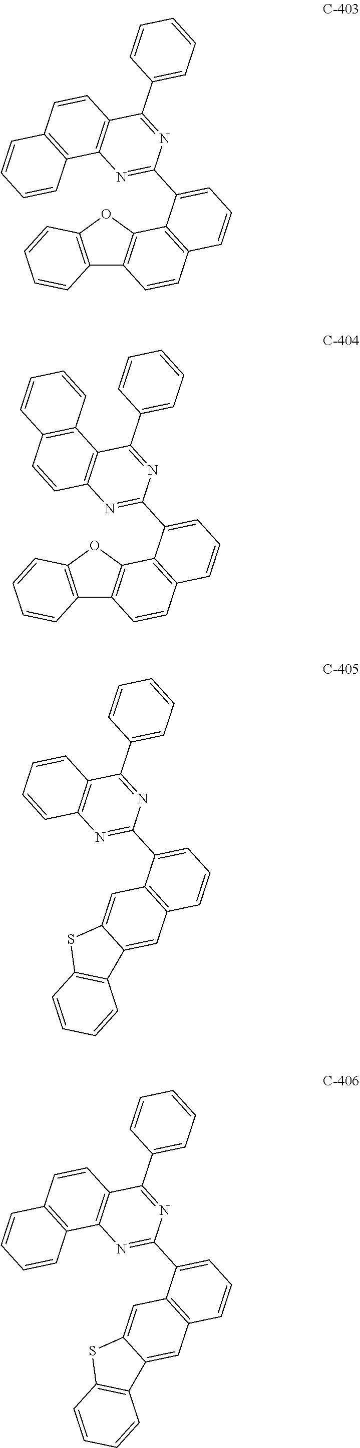 [SMILES: c1ccc(-c2nc(-c3cccc4ccc5c6ccccc6oc5c34)nc3c2ccc2ccccc23)cc1 ; c1ccc(-c2nc(-c3cccc4ccc5c6ccccc6oc5c34)nc3ccc4ccccc4c23)cc1 ; c1ccc(-c2nc(-c3cccc4cc5c(cc34)sc3ccccc35)nc3ccccc23)cc1 ; c1ccc(-c2nc(-c3cccc4cc5c(cc34)sc3ccccc35)nc3c2ccc2ccccc23)cc1]

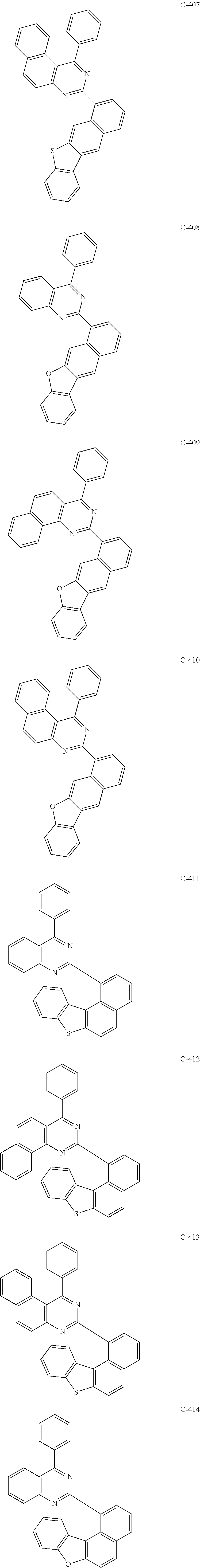

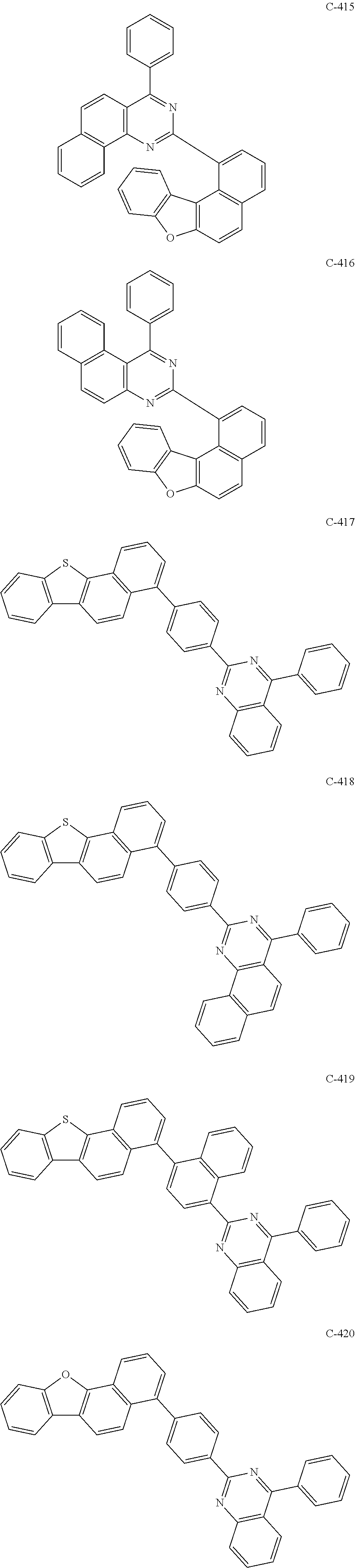 [SMILES: c1ccc(-c2nc(-c3cccc4ccc5oc6ccccc6c5c34)nc3c2ccc2ccccc23)cc1 ; c1ccc(-c2nc(-c3cccc4ccc5oc6ccccc6c5c34)nc3ccc4ccccc4c23)cc1 ; c1ccc(-c2nc(-c3ccc(-c4cccc5c4ccc4c6ccccc6sc54)cc3)nc3ccccc23)cc1 ; c1ccc(-c2nc(-c3ccc(-c4cccc5c4ccc4c6ccccc6sc54)cc3)nc3c2ccc2ccccc23)cc1 ; c1ccc(-c2nc(-c3ccc(-c4cccc5c4ccc4c6ccccc6sc54)c4ccccc34)nc3ccccc23)cc1 ; c1ccc(-c2nc(-c3ccc(-c4cccc5c4ccc4c6ccccc6oc54)cc3)nc3ccccc23)cc1]

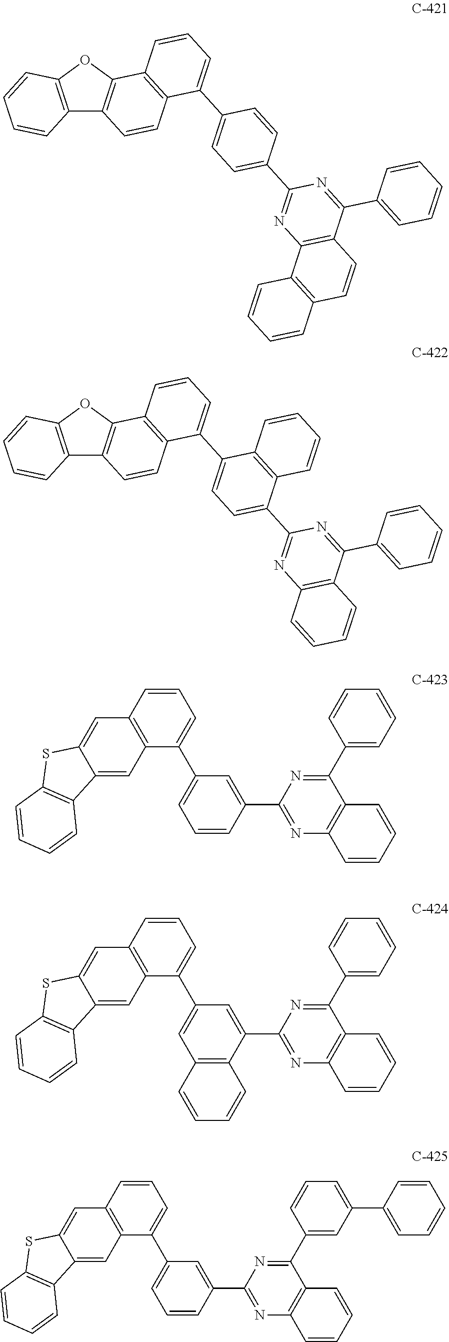 [SMILES: c1ccc(-c2nc(-c3ccc(-c4cccc5c4ccc4c6ccccc6oc54)cc3)nc3c2ccc2ccccc23)cc1 ; c1ccc(-c2nc(-c3ccc(-c4cccc5c4ccc4c6ccccc6oc54)c4ccccc34)nc3ccccc23)cc1 ; c1ccc(-c2nc(-c3cccc(-c4cccc5cc6sc7ccccc7c6cc45)c3)nc3ccccc23)cc1 ; c1ccc(-c2nc(-c3cc(-c4cccc5cc6sc7ccccc7c6cc45)cc4ccccc34)nc3ccccc23)cc1 ; c1ccc(-c2cccc(-c3nc(-c4cccc(-c5cccc6cc7sc8ccccc8c7cc56)c4)nc4ccccc34)c2)cc1]

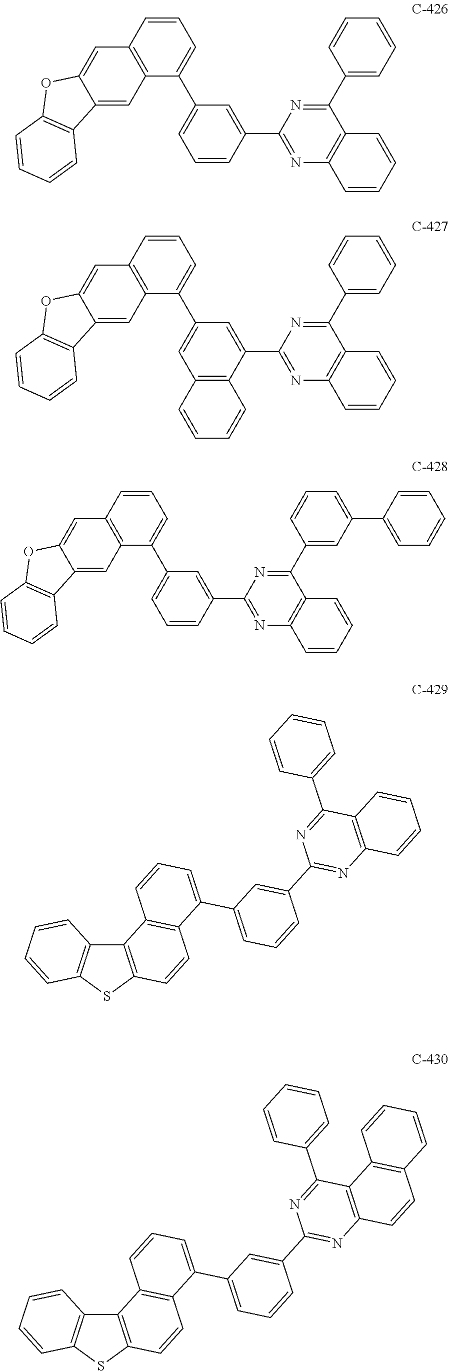

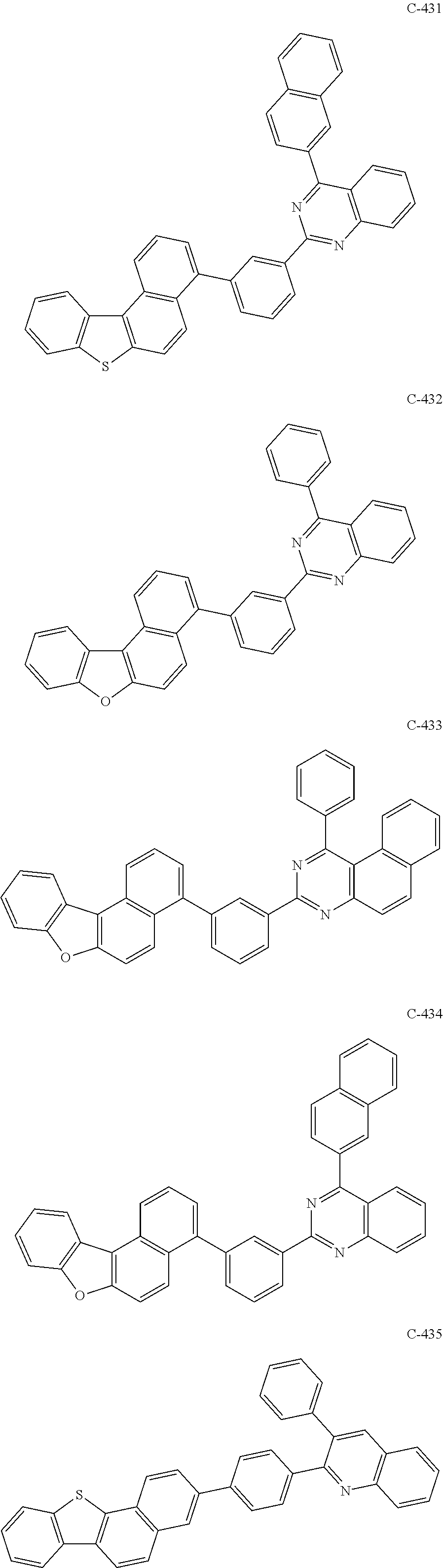 [SMILES: c1cc(-c2nc(-c3ccc4ccccc4c3)c3ccccc3n2)cc(-c2cccc3c2ccc2sc4ccccc4c23)c1 ; c1ccc(-c2nc(-c3cccc(-c4cccc5c4ccc4oc6ccccc6c45)c3)nc3ccccc23)cc1 ; c1ccc(-c2nc(-c3cccc(-c4cccc5c4ccc4oc6ccccc6c45)c3)nc3ccc4ccccc4c23)cc1 ; c1cc(-c2nc(-c3ccc4ccccc4c3)c3ccccc3n2)cc(-c2cccc3c2ccc2oc4ccccc4c23)c1 ; c1ccc(-c2cc3ccccc3nc2-c2ccc(-c3ccc4c(ccc5c6ccccc6sc45)c3)cc2)cc1]

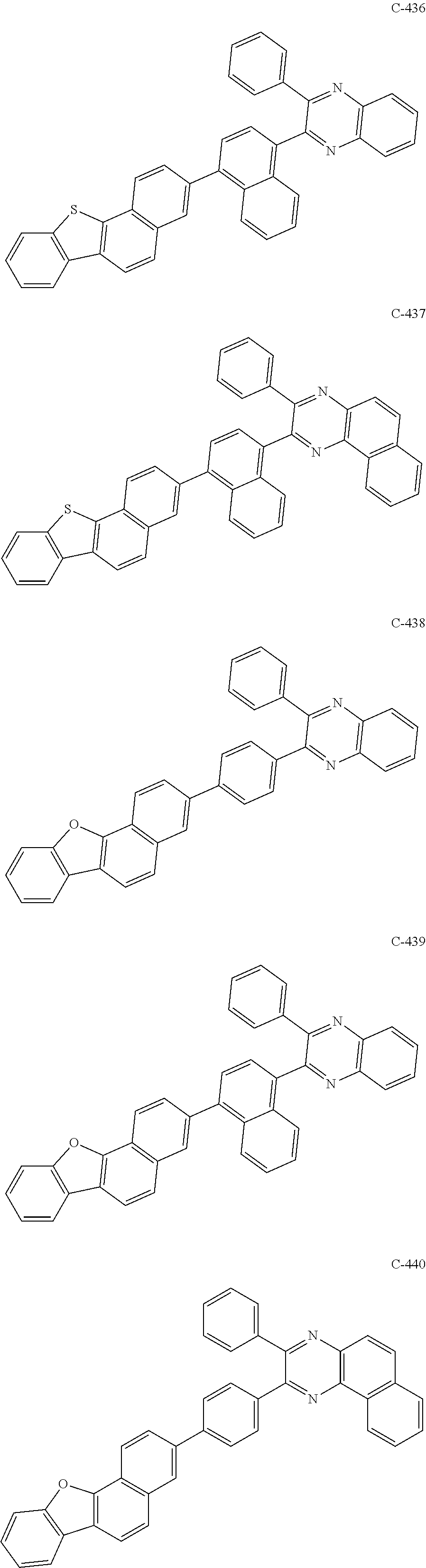 [SMILES: c1ccc(-c2nc3ccccc3nc2-c2ccc(-c3ccc4c(ccc5c6ccccc6sc45)c3)c3ccccc23)cc1 ; c1ccc(-c2nc3ccc4ccccc4c3nc2-c2ccc(-c3ccc4c(ccc5c6ccccc6sc45)c3)c3ccccc23)cc1 ; c1ccc(-c2nc3ccccc3nc2-c2ccc(-c3ccc4c(ccc5c6ccccc6oc45)c3)cc2)cc1 ; c1ccc(-c2nc3ccccc3nc2-c2ccc(-c3ccc4c(ccc5c6ccccc6oc45)c3)c3ccccc23)cc1 ; c1ccc(-c2nc3ccc4ccccc4c3nc2-c2ccc(-c3ccc4c(ccc5c6ccccc6oc45)c3)cc2)cc1]

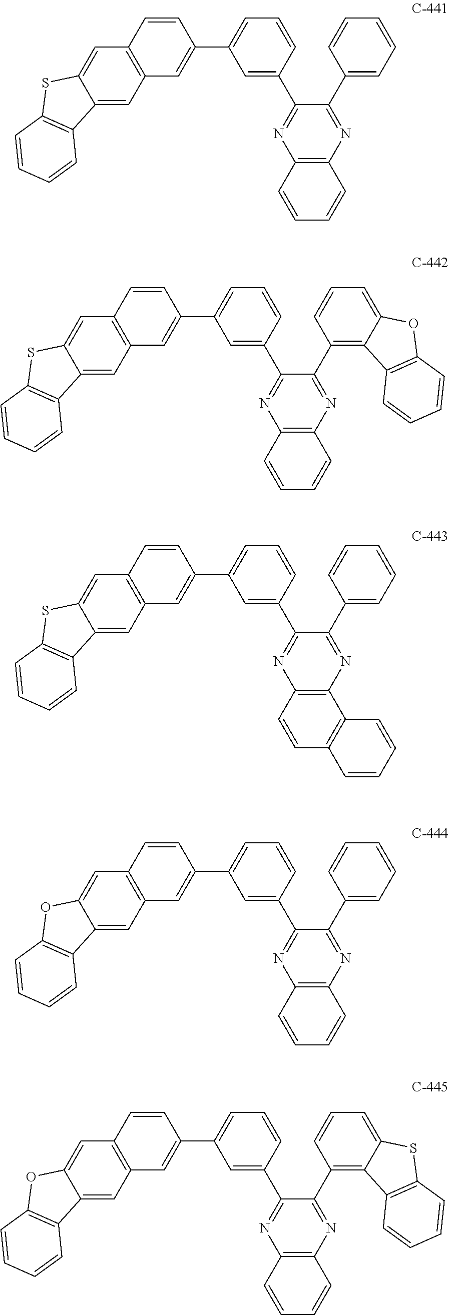 [SMILES: c1ccc(-c2nc3ccccc3nc2-c2cccc(-c3ccc4cc5sc6ccccc6c5cc4c3)c2)cc1 ; c1cc(-c2ccc3cc4sc5ccccc5c4cc3c2)cc(-c2nc3ccccc3nc2-c2cccc3oc4ccccc4c23)c1 ; c1ccc(-c2nc3c(ccc4ccccc43)nc2-c2cccc(-c3ccc4cc5sc6ccccc6c5cc4c3)c2)cc1 ; c1ccc(-c2nc3ccccc3nc2-c2cccc(-c3ccc4cc5oc6ccccc6c5cc4c3)c2)cc1 ; c1cc(-c2ccc3cc4oc5ccccc5c4cc3c2)cc(-c2nc3ccccc3nc2-c2cccc3sc4ccccc4c23)c1]

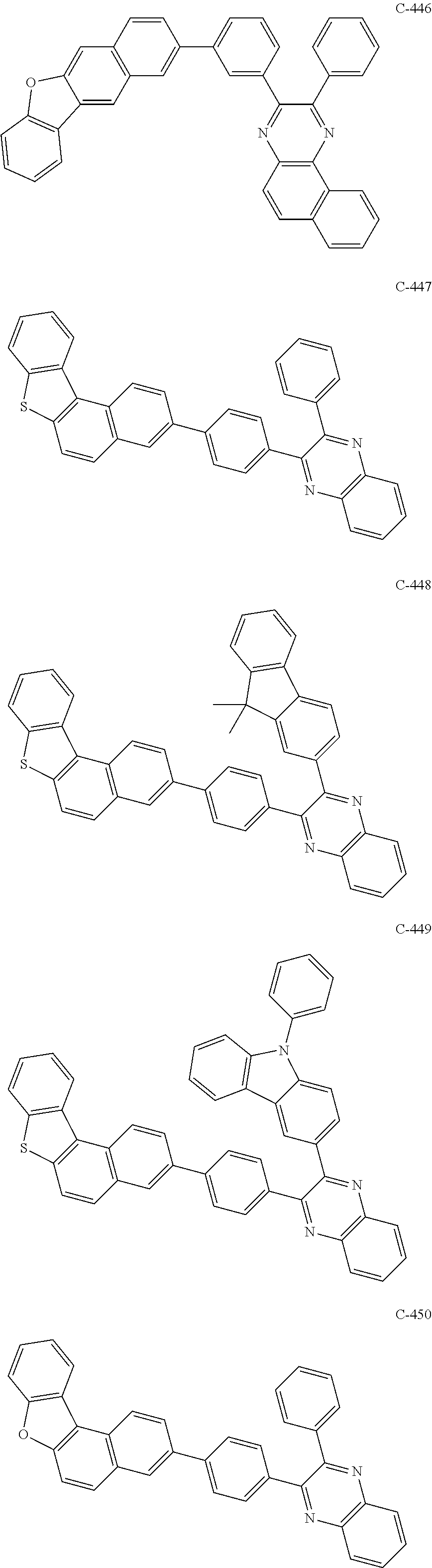 [SMILES: c1ccc(-c2nc3c(ccc4ccccc43)nc2-c2cccc(-c3ccc4cc5oc6ccccc6c5cc4c3)c2)cc1 ; c1ccc(-c2nc3ccccc3nc2-c2ccc(-c3ccc4c(ccc5sc6ccccc6c54)c3)cc2)cc1 ; CC1(C)c2ccccc2-c2ccc(-c3nc4ccccc4nc3-c3ccc(-c4ccc5c(ccc6sc7ccccc7c65)c4)cc3)cc21 ; c1ccc(-n2c3ccccc3c3cc(-c4nc5ccccc5nc4-c4ccc(-c5ccc6c(ccc7sc8ccccc8c76)c5)cc4)ccc32)cc1 ; c1ccc(-c2nc3ccccc3nc2-c2ccc(-c3ccc4c(ccc5oc6ccccc6c54)c3)cc2)cc1]

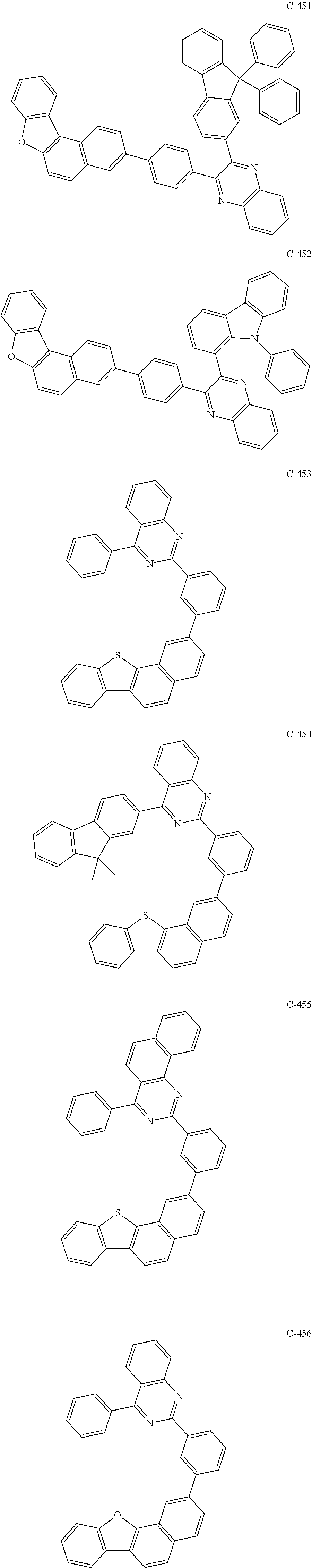 [SMILES: c1ccc(C2(c3ccccc3)c3ccccc3-c3ccc(-c4nc5ccccc5nc4-c4ccc(-c5ccc6c(ccc7oc8ccccc8c76)c5)cc4)cc32)cc1 ; c1ccc(-n2c3ccccc3c3cccc(-c4nc5ccccc5nc4-c4ccc(-c5ccc6c(ccc7oc8ccccc8c76)c5)cc4)c32)cc1 ; c1ccc(-c2nc(-c3cccc(-c4ccc5ccc6c7ccccc7sc6c5c4)c3)nc3ccccc23)cc1 ; CC1(C)c2ccccc2-c2ccc(-c3nc(-c4cccc(-c5ccc6ccc7c8ccccc8sc7c6c5)c4)nc4ccccc34)cc21 ; c1ccc(-c2nc(-c3cccc(-c4ccc5ccc6c7ccccc7sc6c5c4)c3)nc3c2ccc2ccccc23)cc1 ; c1ccc(-c2nc(-c3cccc(-c4ccc5ccc6c7ccccc7oc6c5c4)c3)nc3ccccc23)cc1]

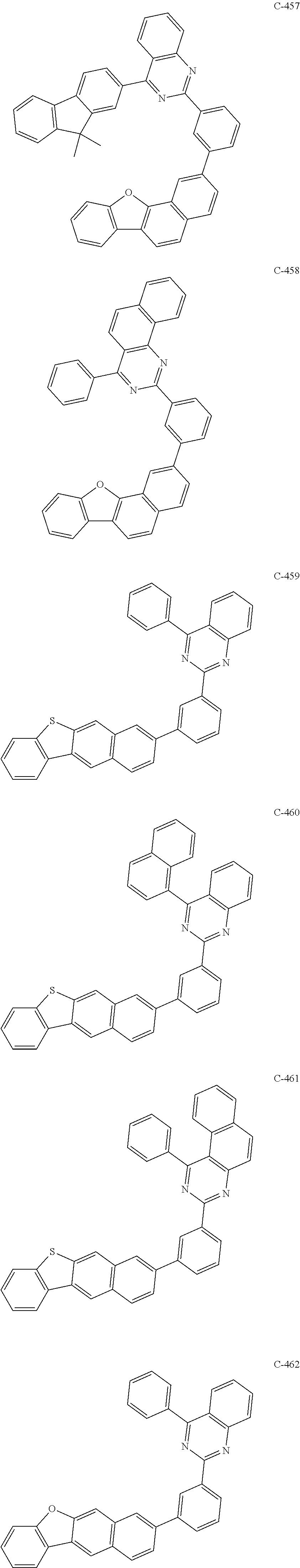

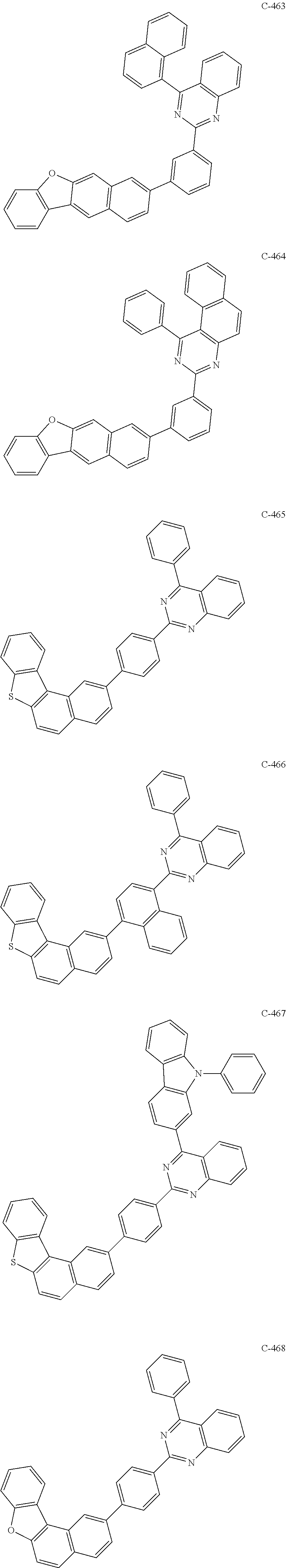 [SMILES: c1cc(-c2ccc3cc4c(cc3c2)oc2ccccc24)cc(-c2nc(-c3cccc4ccccc34)c3ccccc3n2)c1 ; c1ccc(-c2nc(-c3cccc(-c4ccc5cc6c(cc5c4)oc4ccccc46)c3)nc3ccc4ccccc4c23)cc1 ; c1ccc(-c2nc(-c3ccc(-c4ccc5ccc6sc7ccccc7c6c5c4)cc3)nc3ccccc23)cc1 ; c1ccc(-c2nc(-c3ccc(-c4ccc5ccc6sc7ccccc7c6c5c4)c4ccccc34)nc3ccccc23)cc1 ; c1ccc(-n2c3ccccc3c3ccc(-c4nc(-c5ccc(-c6ccc7ccc8sc9ccccc9c8c7c6)cc5)nc5ccccc45)cc32)cc1 ; c1ccc(-c2nc(-c3ccc(-c4ccc5ccc6oc7ccccc7c6c5c4)cc3)nc3ccccc23)cc1]

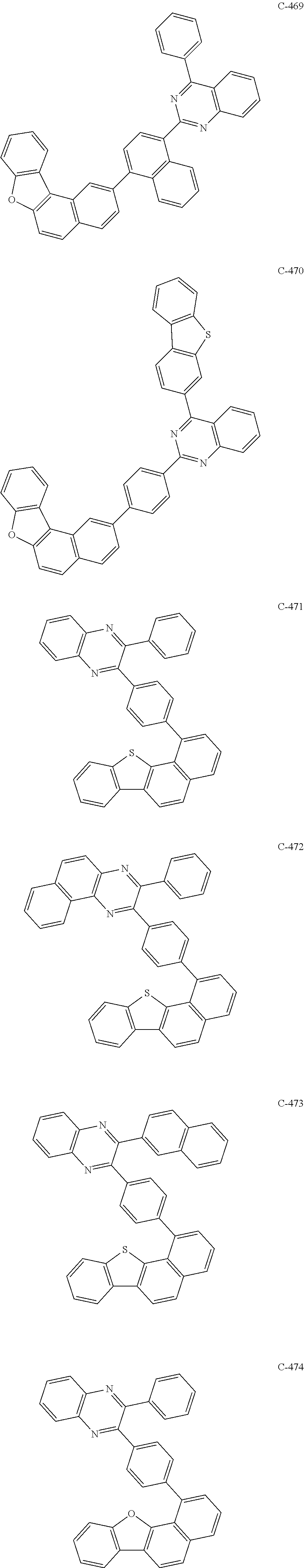 [SMILES: c1ccc(-c2nc(-c3ccc(-c4ccc5ccc6oc7ccccc7c6c5c4)c4ccccc34)nc3ccccc23)cc1 ; c1ccc2c(-c3ccc4c(c3)sc3ccccc34)nc(-c3ccc(-c4ccc5ccc6oc7ccccc7c6c5c4)cc3)nc2c1 ; c1ccc(-c2nc3ccccc3nc2-c2ccc(-c3cccc4ccc5c6ccccc6sc5c34)cc2)cc1 ; c1ccc(-c2nc3ccc4ccccc4c3nc2-c2ccc(-c3cccc4ccc5c6ccccc6sc5c34)cc2)cc1 ; c1ccc2cc(-c3nc4ccccc4nc3-c3ccc(-c4cccc5ccc6c7ccccc7sc6c45)cc3)ccc2c1 ; c1ccc(-c2nc3ccccc3nc2-c2ccc(-c3cccc4ccc5c6ccccc6oc5c34)cc2)cc1]

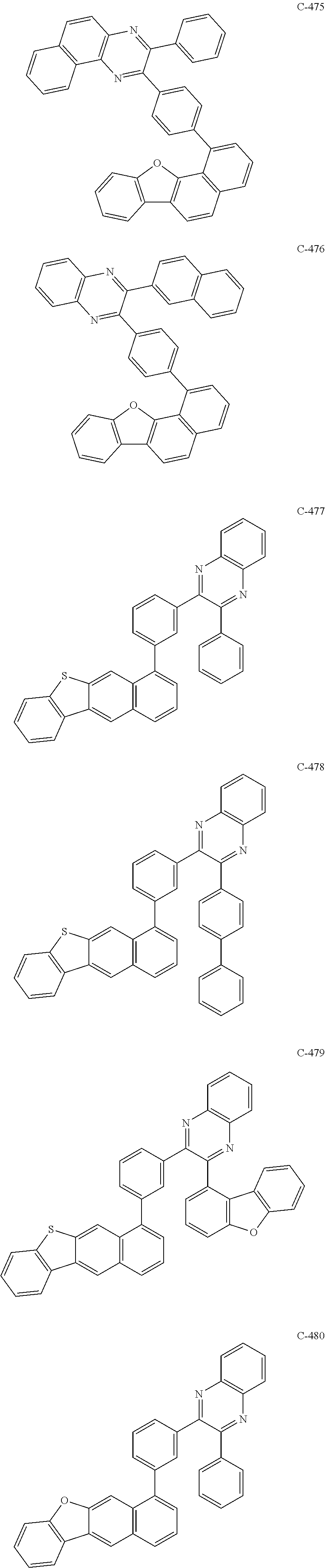

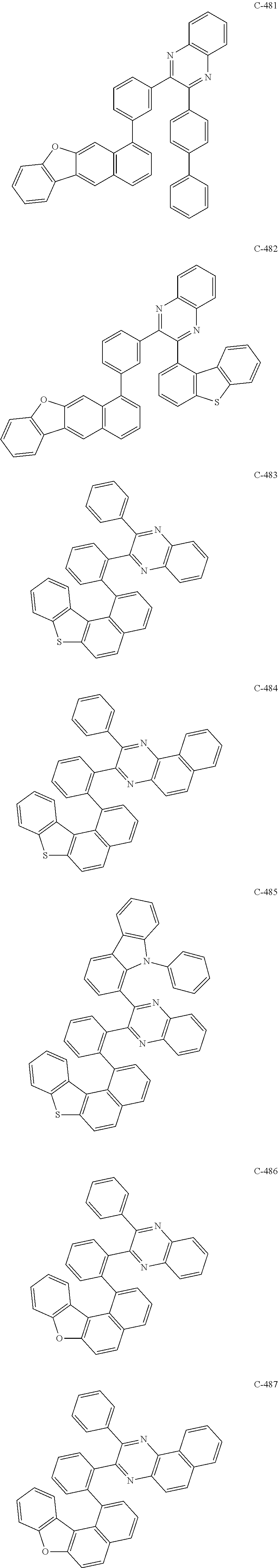

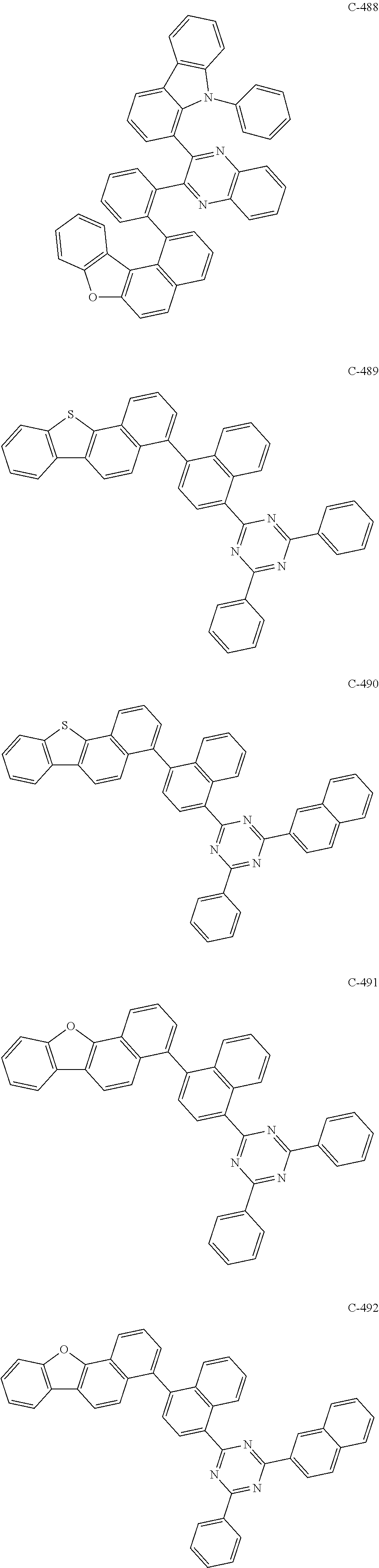 [SMILES: c1ccc(-n2c3ccccc3c3cccc(-c4nc5ccccc5nc4-c4ccccc4-c4cccc5ccc6oc7ccccc7c6c45)c32)cc1 ; c1ccc(-c2nc(-c3ccccc3)nc(-c3ccc(-c4cccc5c4ccc4c6ccccc6sc54)c4ccccc34)n2)cc1 ; c1ccc(-c2nc(-c3ccc4ccccc4c3)nc(-c3ccc(-c4cccc5c4ccc4c6ccccc6sc54)c4ccccc34)n2)cc1 ; c1ccc(-c2nc(-c3ccccc3)nc(-c3ccc(-c4cccc5c4ccc4c6ccccc6oc54)c4ccccc34)n2)cc1 ; c1ccc(-c2nc(-c3ccc4ccccc4c3)nc(-c3ccc(-c4cccc5c4ccc4c6ccccc6oc54)c4ccccc34)n2)cc1]

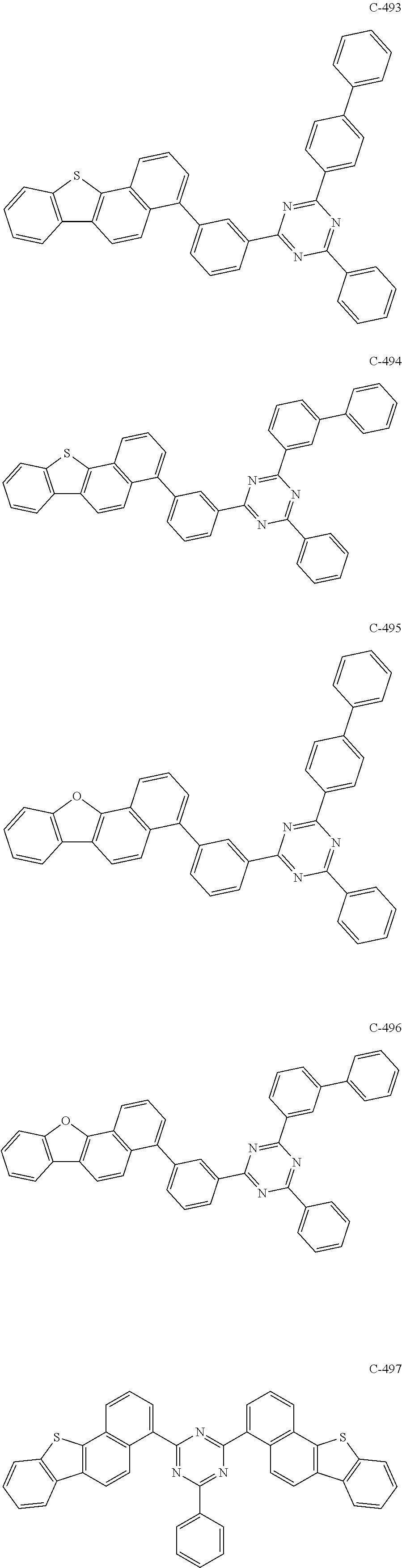 [SMILES: c1ccc(-c2ccc(-c3nc(-c4ccccc4)nc(-c4cccc(-c5cccc6c5ccc5c7ccccc7sc65)c4)n3)cc2)cc1 ; c1ccc(-c2cccc(-c3nc(-c4ccccc4)nc(-c4cccc(-c5cccc6c5ccc5c7ccccc7sc65)c4)n3)c2)cc1 ; c1ccc(-c2ccc(-c3nc(-c4ccccc4)nc(-c4cccc(-c5cccc6c5ccc5c7ccccc7oc65)c4)n3)cc2)cc1 ; c1ccc(-c2cccc(-c3nc(-c4ccccc4)nc(-c4cccc(-c5cccc6c5ccc5c7ccccc7oc65)c4)n3)c2)cc1 ; c1ccc(-c2nc(-c3cccc4c3ccc3c5ccccc5sc43)nc(-c3cccc4c3ccc3c5ccccc5sc43)n2)cc1]

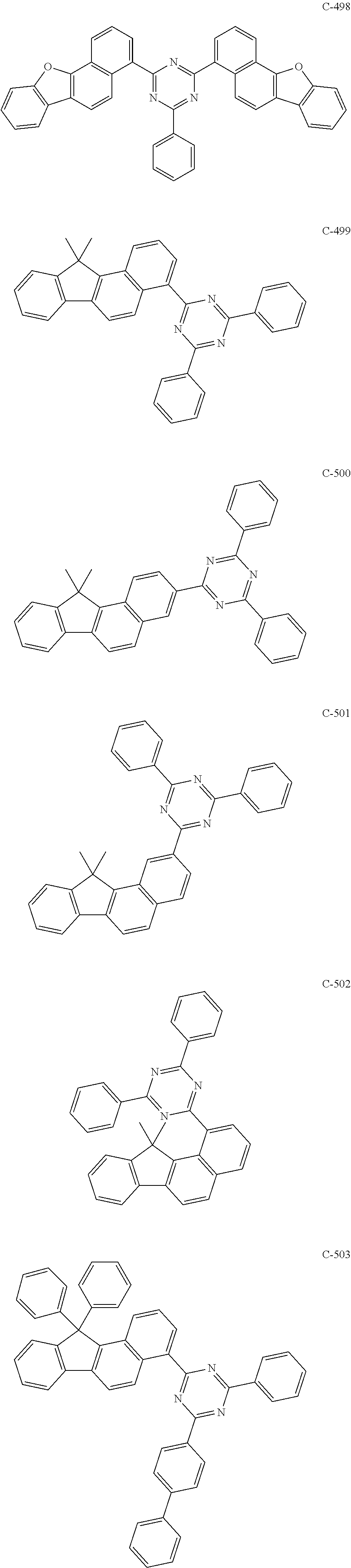 [SMILES: c1ccc(-c2nc(-c3cccc4c3ccc3c5ccccc5oc43)nc(-c3cccc4c3ccc3c5ccccc5oc43)n2)cc1 ; CC1(C)c2ccccc2-c2ccc3c(-c4nc(-c5ccccc5)nc(-c5ccccc5)n4)cccc3c21 ; CC1(C)c2ccccc2-c2ccc3cc(-c4nc(-c5ccccc5)nc(-c5ccccc5)n4)ccc3c21 ; CC1(C)c2ccccc2-c2ccc3ccc(-c4nc(-c5ccccc5)nc(-c5ccccc5)n4)cc3c21 ; CC1(C)c2ccccc2-c2ccc3cccc(-c4nc(-c5ccccc5)nc(-c5ccccc5)n4)c3c21 ; c1ccc(-c2ccc(-c3nc(-c4ccccc4)nc(-c4cccc5c6c(ccc45)-c4ccccc4C6(c4ccccc4)c4ccccc4)n3)cc2)cc1]

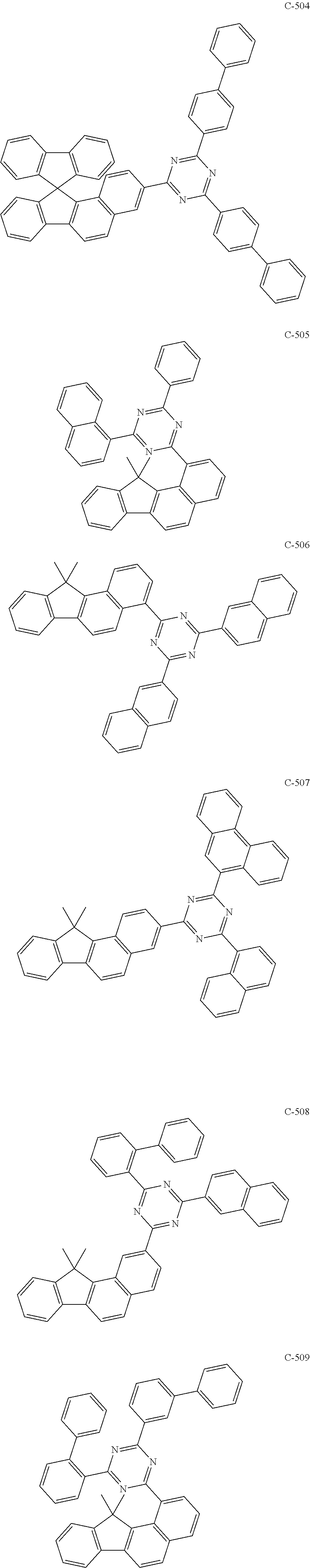

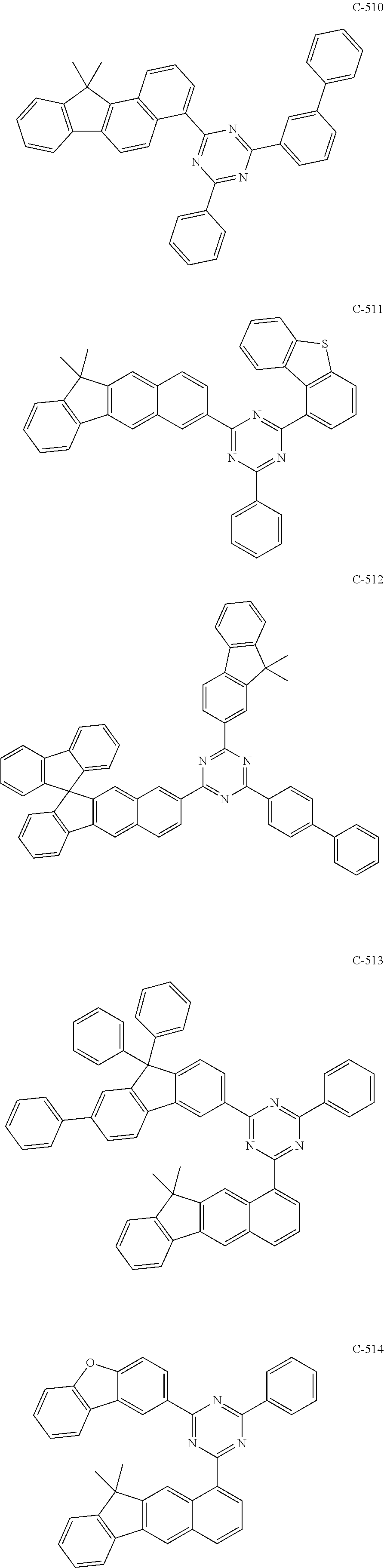 [SMILES: CC1(C)c2ccccc2-c2ccc3c(-c4nc(-c5ccccc5)nc(-c5cccc(-c6ccccc6)c5)n4)cccc3c21 ; CC1(C)c2ccccc2-c2cc3cc(-c4nc(-c5ccccc5)nc(-c5cccc6sc7ccccc7c56)n4)ccc3cc21 ; CC1(C)c2ccccc2-c2ccc(-c3nc(-c4ccc(-c5ccccc5)cc4)nc(-c4ccc5cc6c(cc5c4)C4(c5ccccc5-c5ccccc54)c4ccccc4-6)n3)cc21 ; CC1(C)c2ccccc2-c2cc3cccc(-c4nc(-c5ccccc5)nc(-c5ccc6c(c5)-c5ccc(-c7ccccc7)cc5C6(c5ccccc5)c5ccccc5)n4)c3cc21 ; CC1(C)c2ccccc2-c2cc3cccc(-c4nc(-c5ccccc5)nc(-c5ccc6oc7ccccc7c6c5)n4)c3cc21]

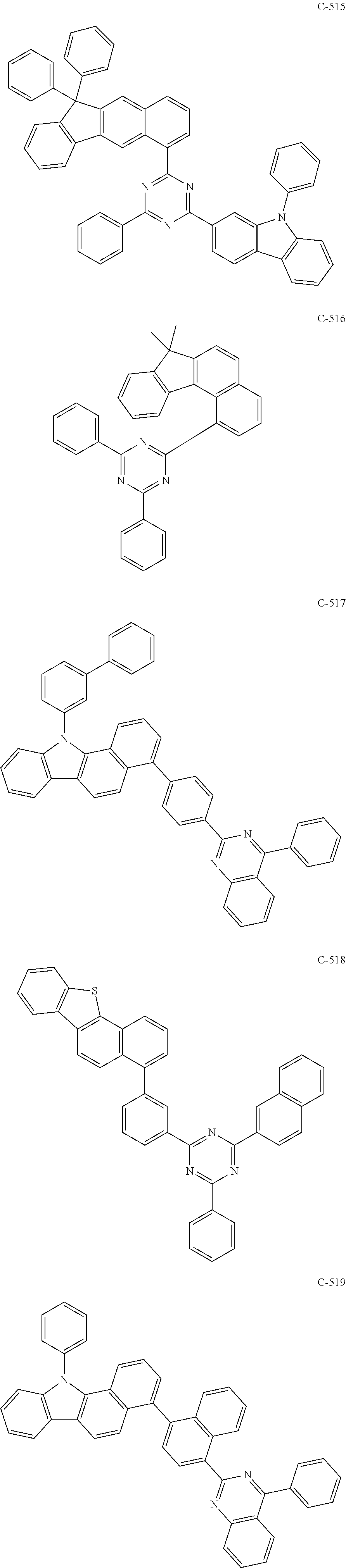

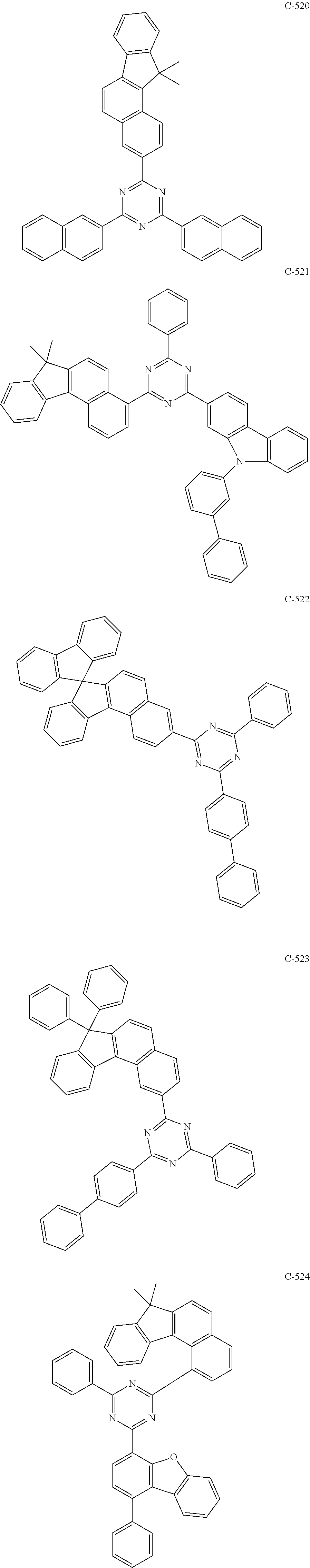 [SMILES: CC1(C)c2ccccc2-c2ccc3cc(-c4nc(-c5ccc6ccccc6c5)nc(-c5ccc6ccccc6c5)n4)ccc3c21 ; CC1(C)c2ccccc2-c2c1ccc1c(-c3nc(-c4ccccc4)nc(-c4ccc5c6ccccc6n(-c6cccc(-c7ccccc7)c6)c5c4)n3)cccc21 ; c1ccc(-c2ccc(-c3nc(-c4ccccc4)nc(-c4ccc5c6c(ccc5c4)C4(c5ccccc5-c5ccccc54)c4ccccc4-6)n3)cc2)cc1 ; c1ccc(-c2ccc(-c3nc(-c4ccccc4)nc(-c4ccc5ccc6c(c5c4)-c4ccccc4C6(c4ccccc4)c4ccccc4)n3)cc2)cc1 ; CC1(C)c2ccccc2-c2c1ccc1cccc(-c3nc(-c4ccccc4)nc(-c4ccc(-c5ccccc5)c5c4oc4ccccc45)n3)c21]

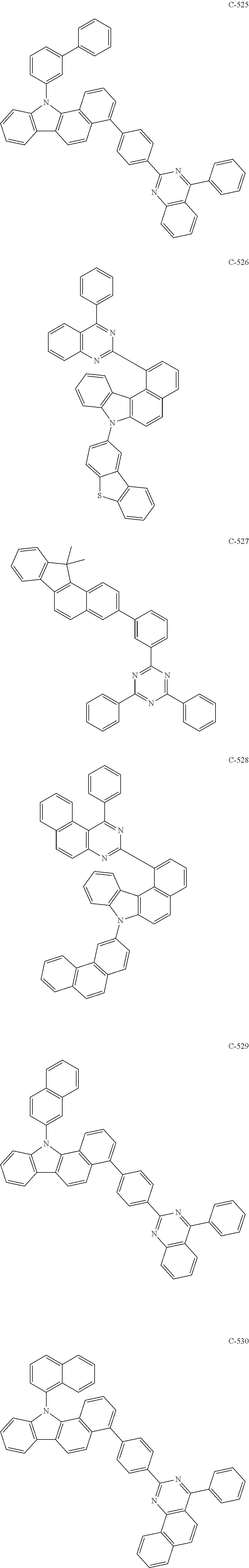 [SMILES: c1ccc(-c2cccc(-n3c4ccccc4c4ccc5c(-c6ccc(-c7nc(-c8ccccc8)c8ccccc8n7)cc6)cccc5c43)c2)cc1 ; c1ccc(-c2nc(-c3cccc4ccc5c(c6ccccc6n5-c5ccc6sc7ccccc7c6c5)c34)nc3ccccc23)cc1 ; CC1(C)c2ccccc2-c2ccc3cc(-c4cccc(-c5nc(-c6ccccc6)nc(-c6ccccc6)n5)c4)ccc3c21 ; c1ccc(-c2nc(-c3cccc4ccc5c(c6ccccc6n5-c5ccc6ccc7ccccc7c6c5)c34)nc3ccc4ccccc4c23)cc1 ; c1ccc(-c2nc(-c3ccc(-c4cccc5c4ccc4c6ccccc6n(-c6ccc7ccccc7c6)c54)cc3)nc3ccccc23)cc1 ; c1ccc(-c2nc(-c3ccc(-c4cccc5c4ccc4c6ccccc6n(-c6cccc7ccccc67)c54)cc3)nc3c2ccc2ccccc23)cc1]

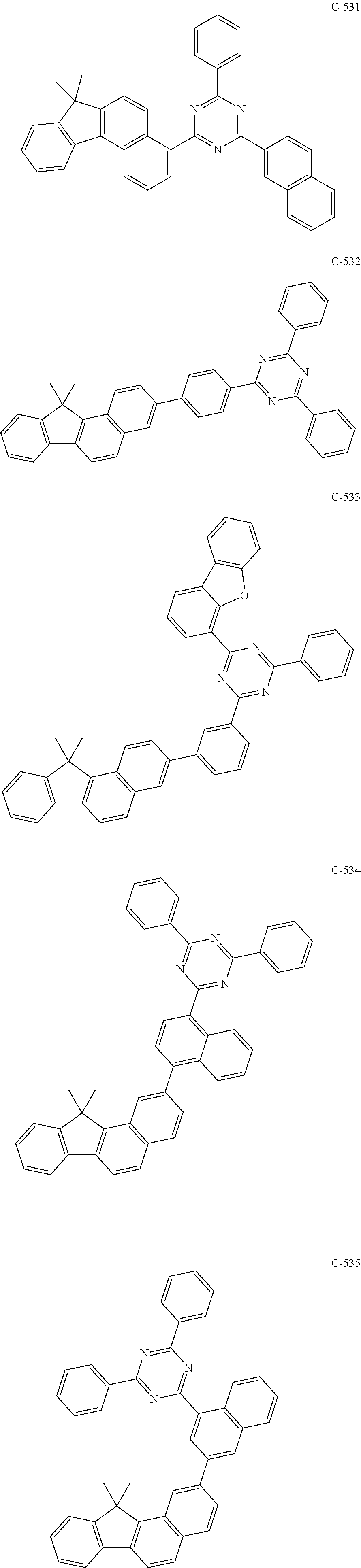 [SMILES: CC1(C)c2ccccc2-c2c1ccc1c(-c3nc(-c4ccccc4)nc(-c4ccc5ccccc5c4)n3)cccc21 ; CC1(C)c2ccccc2-c2ccc3cc(-c4ccc(-c5nc(-c6ccccc6)nc(-c6ccccc6)n5)cc4)ccc3c21 ; CC1(C)c2ccccc2-c2ccc3cc(-c4cccc(-c5nc(-c6ccccc6)nc(-c6cccc7c6oc6ccccc67)n5)c4)ccc3c21 ; CC1(C)c2ccccc2-c2ccc3ccc(-c4ccc(-c5nc(-c6ccccc6)nc(-c6ccccc6)n5)c5ccccc45)cc3c21 ; CC1(C)c2ccccc2-c2ccc3ccc(-c4cc(-c5nc(-c6ccccc6)nc(-c6ccccc6)n5)c5ccccc5c4)cc3c21]

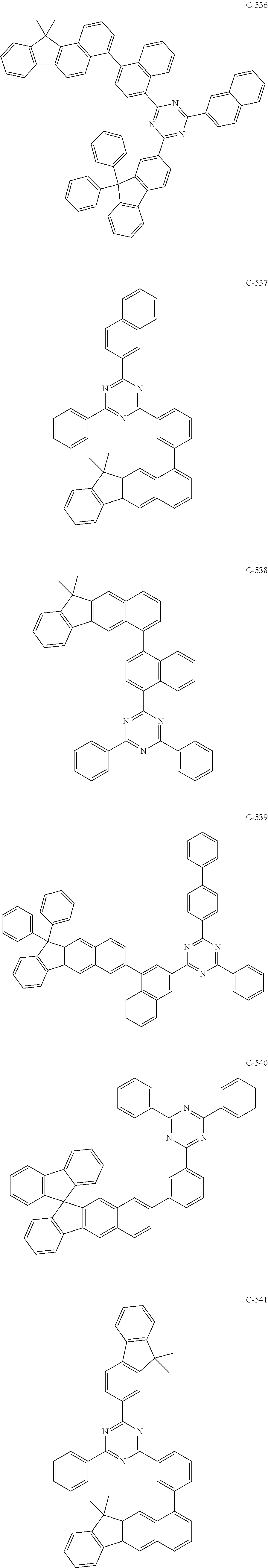

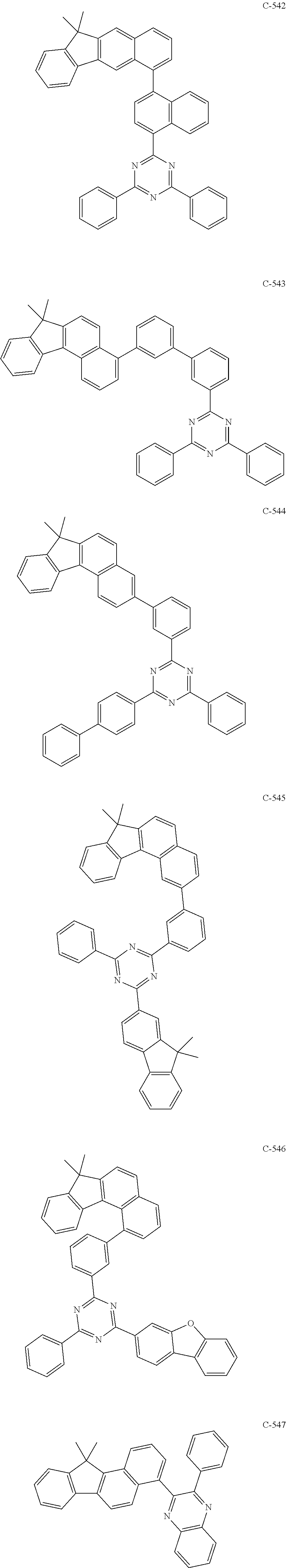 [SMILES: CC1(C)c2ccccc2-c2cc3c(-c4ccc(-c5nc(-c6ccccc6)nc(-c6ccccc6)n5)c5ccccc45)cccc3cc21 ; CC1(C)c2ccccc2-c2c1ccc1c(-c3cccc(-c4cccc(-c5nc(-c6ccccc6)nc(-c6ccccc6)n5)c4)c3)cccc21 ; CC1(C)c2ccccc2-c2c1ccc1cc(-c3cccc(-c4nc(-c5ccccc5)nc(-c5ccc(-c6ccccc6)cc5)n4)c3)ccc21 ; CC1(C)c2ccccc2-c2ccc(-c3nc(-c4ccccc4)nc(-c4cccc(-c5ccc6ccc7c(c6c5)-c5ccccc5C7(C)C)c4)n3)cc21 ; CC1(C)c2ccccc2-c2c1ccc1cccc(-c3cccc(-c4nc(-c5ccccc5)nc(-c5ccc6c(c5)oc5ccccc56)n4)c3)c21 ; CC1(C)c2ccccc2-c2ccc3c(-c4nc5ccccc5nc4-c4ccccc4)cccc3c21]

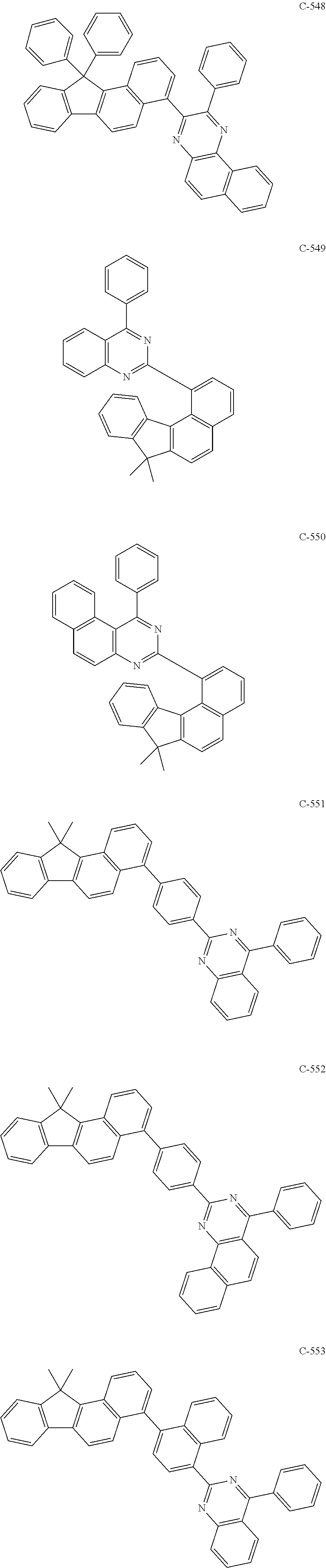 [SMILES: c1ccc(-c2nc3c(ccc4ccccc43)nc2-c2cccc3c4c(ccc23)-c2ccccc2C4(c2ccccc2)c2ccccc2)cc1 ; CC1(C)c2ccccc2-c2c1ccc1cccc(-c3nc(-c4ccccc4)c4ccccc4n3)c21 ; CC1(C)c2ccccc2-c2c1ccc1cccc(-c3nc(-c4ccccc4)c4c(ccc5ccccc54)n3)c21 ; CC1(C)c2ccccc2-c2ccc3c(-c4ccc(-c5nc(-c6ccccc6)c6ccccc6n5)cc4)cccc3c21 ; CC1(C)c2ccccc2-c2ccc3c(-c4ccc(-c5nc(-c6ccccc6)c6ccc7ccccc7c6n5)cc4)cccc3c21 ; CC1(C)c2ccccc2-c2ccc3c(-c4ccc(-c5nc(-c6ccccc6)c6ccccc6n5)c5ccccc45)cccc3c21]

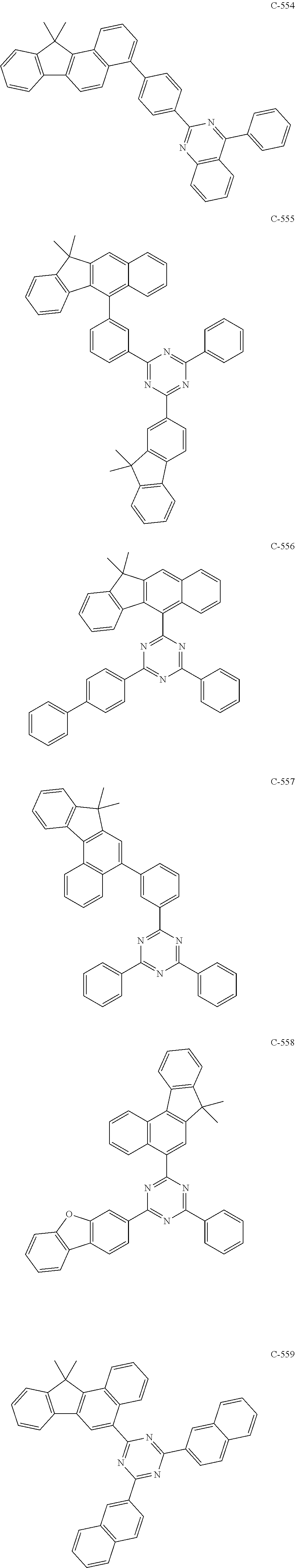 [SMILES: CC1(C)c2ccccc2-c2ccc3c(-c4ccc(-c5nc(-c6ccccc6)c6ccccc6n5)cc4)cccc3c21 ; CC1(C)c2ccccc2-c2ccc(-c3nc(-c4ccccc4)nc(-c4cccc(-c5c6c(cc7ccccc57)C(C)(C)c5ccccc5-6)c4)n3)cc21 ; CC1(C)c2ccccc2-c2c1cc1ccccc1c2-c1nc(-c2ccccc2)nc(-c2ccc(-c3ccccc3)cc2)n1 ; CC1(C)c2ccccc2-c2c1cc(-c1cccc(-c3nc(-c4ccccc4)nc(-c4ccccc4)n3)c1)c1ccccc21 ; CC1(C)c2ccccc2-c2c1cc(-c1nc(-c3ccccc3)nc(-c3ccc4c(c3)oc3ccccc34)n1)c1ccccc21 ; CC1(C)c2ccccc2-c2cc(-c3nc(-c4ccc5ccccc5c4)nc(-c4ccc5ccccc5c4)n3)c3ccccc3c21]

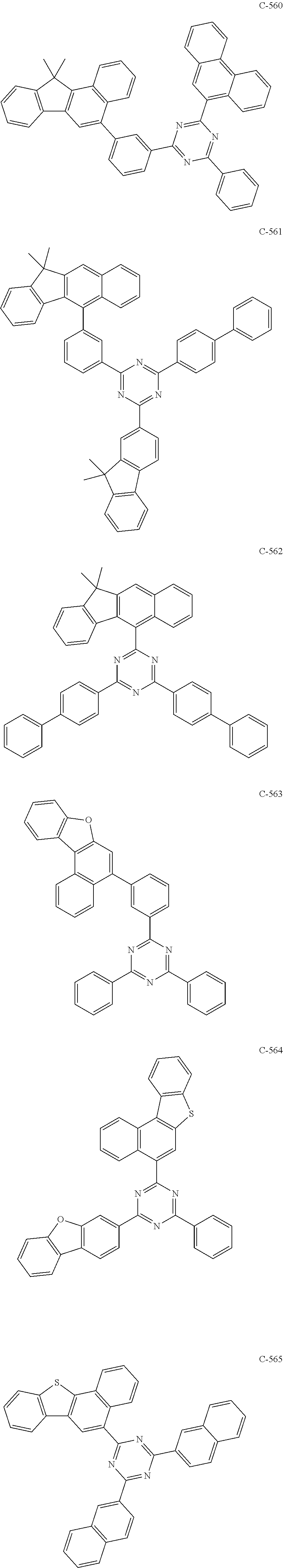 [SMILES: CC1(C)c2ccccc2-c2cc(-c3cccc(-c4nc(-c5ccccc5)nc(-c5cc6ccccc6c6ccccc56)n4)c3)c3ccccc3c21 ; CC1(C)c2ccccc2-c2ccc(-c3nc(-c4ccc(-c5ccccc5)cc4)nc(-c4cccc(-c5c6c(cc7ccccc57)C(C)(C)c5ccccc5-6)c4)n3)cc21 ; CC1(C)c2ccccc2-c2c1cc1ccccc1c2-c1nc(-c2ccc(-c3ccccc3)cc2)nc(-c2ccc(-c3ccccc3)cc2)n1 ; c1ccc(-c2nc(-c3ccccc3)nc(-c3cccc(-c4cc5oc6ccccc6c5c5ccccc45)c3)n2)cc1 ; c1ccc(-c2nc(-c3ccc4c(c3)oc3ccccc34)nc(-c3cc4sc5ccccc5c4c4ccccc34)n2)cc1 ; c1ccc2cc(-c3nc(-c4ccc5ccccc5c4)nc(-c4cc5c6ccccc6sc5c5ccccc45)n3)ccc2c1]

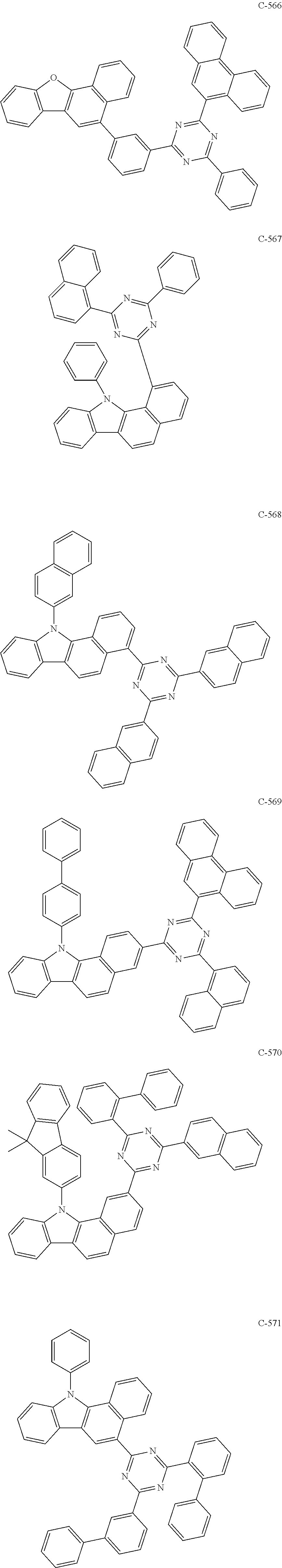

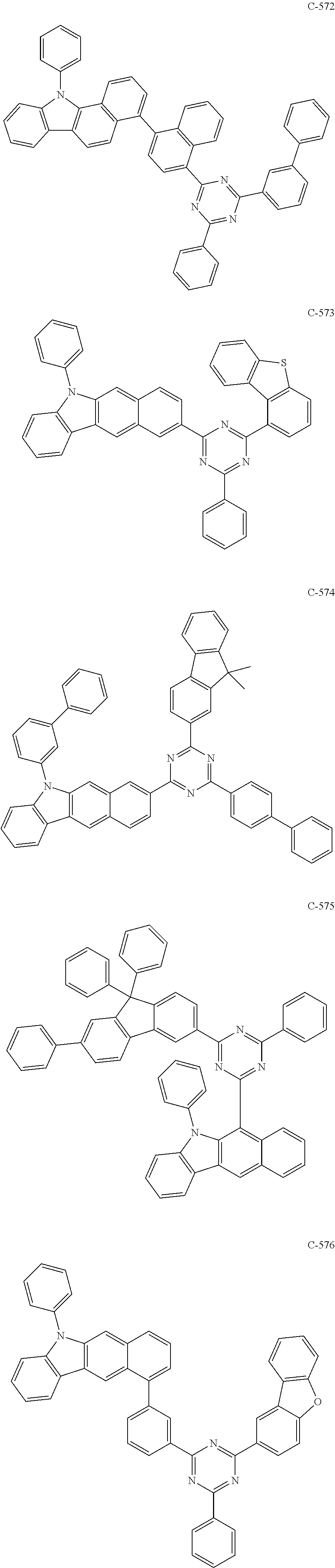

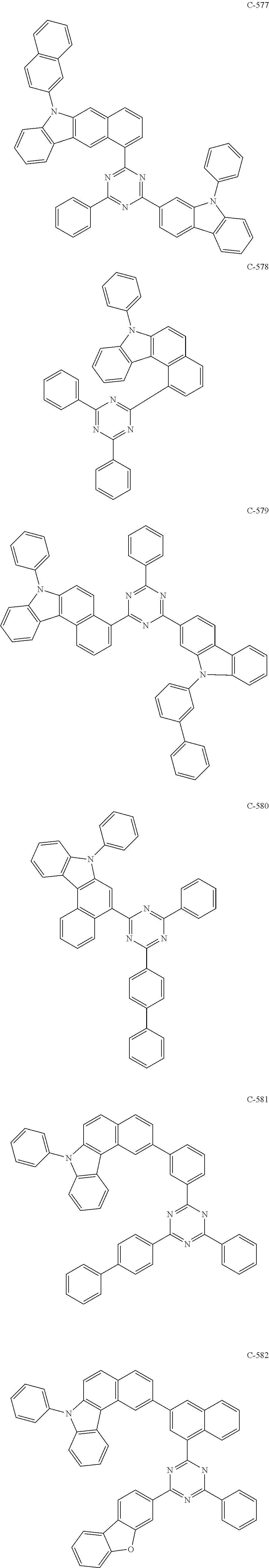

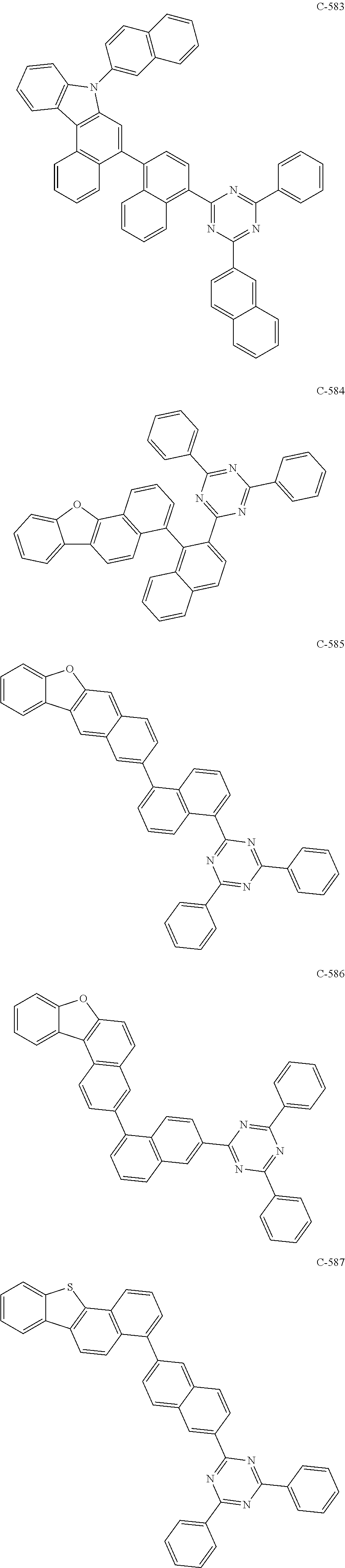 [SMILES: c1ccc(-c2nc(-c3ccc4ccccc4c3)nc(-c3ccc(-c4cc5c(c6ccccc46)c4ccccc4n5-c4ccc5ccccc5c4)c4ccccc34)n2)cc1 ; c1ccc(-c2nc(-c3ccccc3)nc(-c3ccc4ccccc4c3-c3cccc4c3ccc3c5ccccc5oc43)n2)cc1 ; c1ccc(-c2nc(-c3ccccc3)nc(-c3cccc4c(-c5ccc6cc7oc8ccccc8c7cc6c5)cccc34)n2)cc1 ; c1ccc(-c2nc(-c3ccccc3)nc(-c3ccc4c(-c5ccc6c(ccc7oc8ccccc8c76)c5)cccc4c3)n2)cc1 ; c1ccc(-c2nc(-c3ccccc3)nc(-c3ccc4cc(-c5cccc6c5ccc5c7ccccc7sc65)ccc4c3)n2)cc1]

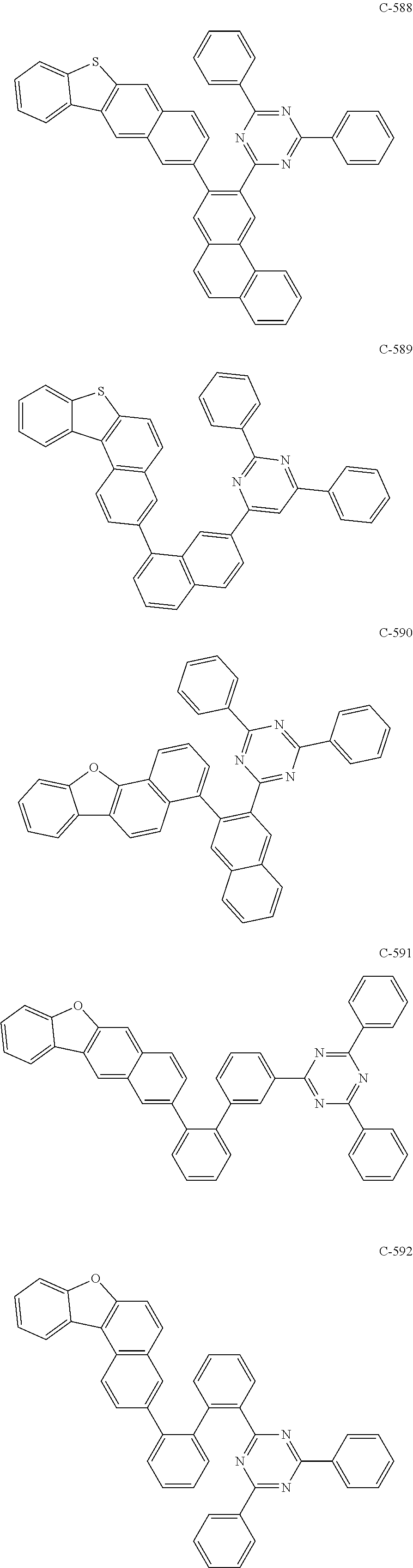 [SMILES: c1ccc(-c2nc(-c3ccccc3)nc(-c3cc4c(ccc5ccccc54)cc3-c3ccc4cc5sc6ccccc6c5cc4c3)n2)cc1 ; c1ccc(-c2cc(-c3ccc4cccc(-c5ccc6c(ccc7sc8ccccc8c76)c5)c4c3)nc(-c3ccccc3)n2)cc1 ; c1ccc(-c2nc(-c3ccccc3)nc(-c3cc4ccccc4cc3-c3cccc4c3ccc3c5ccccc5oc43)n2)cc1 ; c1ccc(-c2nc(-c3ccccc3)nc(-c3cccc(-c4ccccc4-c4ccc5cc6oc7ccccc7c6cc5c4)c3)n2)cc1 ; c1ccc(-c2nc(-c3ccccc3)nc(-c3ccccc3-c3ccccc3-c3ccc4c(ccc5oc6ccccc6c54)c3)n2)cc1]

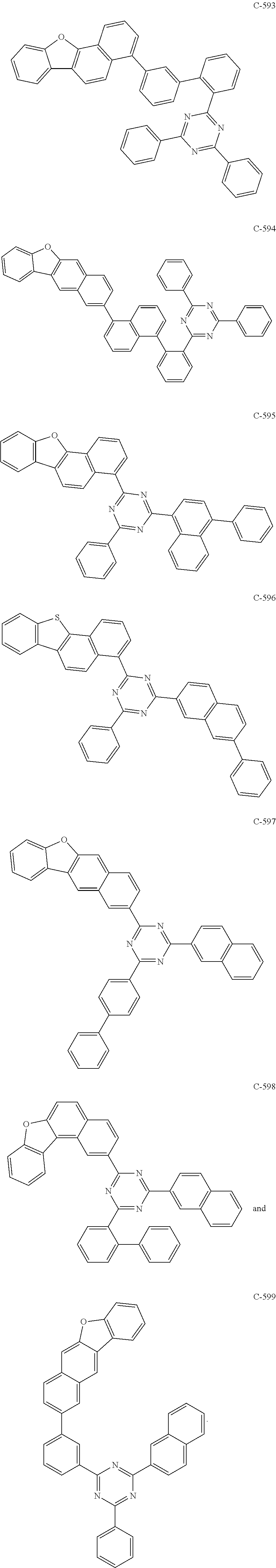

7. An organic electroluminescent device comprising an anode, a cathode, and at least one light-emitting layer between the anode and the cathode, wherein at least one of the light-emitting layers comprises the plurality of host materials according to claim 1.
